SEQUENCE LISTING (439 sequences)

SEQ ID NO: 1 - protein, 499 aa, synthetic construct
MRYVYVLDVDGKPLMPTCRFGKVRRMLKSGQAKAVDTLPFTIQLTYKPRTRILQPVTLGQDPGRTNIGMAAVRFDGKELGRFHCITRNKEIPKLMADRMAARKASRRGERLARKRLARKLHTTAKHLNGRILPGCSEPIAVKDIINTESRFNNRLRPEGWLTPTATQLLRTHINLFRKLSGILPVTDVAVELNKFAFMQLDNPEMKKREIDFCHGPLCGTGGLEAAVKEQQDGKCLLCGKESIGHYHHIVPRSRRGSNTIANIAGLCPKCHELVHKDADTAESLTEMKTGLMKKYGGTSVLNQIIPKLVETLADLFPGHFHVTNGWNTKEFREKHHLEKDHDVDAYCIACSHLKPEETLVETEPFEILQFRKHNRAIIHHQTERTYKLDGVTVAKNRKKRMEQKTDSLEDWYVDMAKEHGKTQADAMRSRLTVIKSTRYYNTPGRMMPGTVFLYEGKRYVMTGQITNGKYYRAYGQEKRNFPAVKVRILTKNTGLVFVA

SEQ ID NO: 2 - protein, 598 aa, synthetic construct
MRYVYVLDVDGKPLMPTCRFGKVRRMLKSGQAKAVDTLPFTIQLTYKPRTRILQPVTLGQDPGRTNIGMAAVRFDGKELGRFHCITRNKEIPKLMADRMAARKASRRGERLARKRLARKLHTTAKHLNGRILPGCSEPIAVKDIINTESRFNNRILTKCKVCGKNTPLRRNVRELLLENIVRFLPLESELKETLKRTILEGQQGNINKLFRKLKFNQKDWPGKNLTDIAKNKLPGRLPFCKEHFAENEKFTTIEKSTFRLTPTATQLLRTHINLFRKLSGILPVTDVAVELNKFAFMQLDNPEMKKREIDFCHGPLCGTGGLEAAVKEQQDGKCLLCGKESIGHYHHIVPRSRRGSNTIANIAGLCPKCHELVHKDADTAESLTEMKTGLMKKYGGTSVLNQIIPKLVETLADLFPGHFHVTNGWNTKEFREKHHLEKDHDVDAYCIACSHLKPEETLVETEPFEILQFRKHNRAIIHHQTERTYKLDGVTVAKNRKKRMEQKTDSLEDWYVDMAKEHGKTQADAMRSRLTVIKSTRYYNTPGRMMPGTVFLYEGKRYVMTGQITNGKYYRAYGQEKRNFPAVKVRILTKNTGLVFVA

SEQ ID NO: 3 - protein, 598 aa, synthetic construct
MRYVYVLDVDGKPLMPTCRFGKVRRMLKSGQAKAVDTLPFTIQLTYKPRTRILQPVTLGQDPGRTNIGMAAVRFDGKELGRFHCITRNKEIPKLMADRMAARKASRRGERLARKRLARKLHTTAKHLNGRILPGCSKPIAVKDIINTESRFNNRILTKCKVCGKNTPLRRNVRELLLENIVRFLPLESELKETLKRTILEGQQGNINKLFRKLKFNQKDWPGKNLTDIAKNKLPGRLPFCKEHFAENEKFTTIEKSTFRLTPTATQLLRTHINLFRKLSGILPVTDVAVELNKFAFMQLDNPEMKKREIDFCHGPLCGTGGLEAAVKEQQDGKCLLCGKESIGHYHHIVPRSRRGSNTIANIAGLCPKCHELVHKDADTAESLTEMKTGLMKKYGGTSVLNQIIPKLVRTLAKLFPGHFHVTNGWNTKEFREKHHLEKDHDVDAYCIACSHLKPEETLVETEPFEILQFRKHNRAIIHHQTERTYKLDGVTVAKNRKKRMEQKTDSLEDWYVDMAKEHGKTQADAMRSRLTVKKSRRYYNTPGRMMPGTVFLYEGKRYVMTGQITNGKYYRAYGQEKRNFPAVKVRILTKNTGLVFVA

SEQ ID NO: 4 - protein, 598 aa, synthetic construct
MRYVYVLDVDGKPLMPTCRFGKVRRMLKSGQAKAVDTLPFTIQLTYKPRTRILQPVTLGQDPGRTHIGMAAVRFDGKELGRFHCITRNKEIPKLMADRMKARKASRRGERLARKRLARKLHTTAKHLNGRILPGCSKPIAVKDIINTESRFNNRILTKCKVCGKNTPLRRNVRELLLENIVRFLPLESELKETLKRTILEGQQGNINKLFRKLKFNQKDWPGKNLTDIAKNKLPGRLPFCKEHFAENEKFTTIEKSTFRLTPTATQLLRTHINLFRKLSGILPVTDVAVELNKFAFMQLDNPEMKKREIDFCHGPLCGTGGLEAAVKEQQDGKCLLCGKESIGHYHHIVPRSRRGSNTIANIAGLCPKCHELVHKDADTAESLTEMKTGLMKKYGGTSVLNQIIPKLVRTLAKLFPGHFHVTNGWNTKEFREKHHLEKDHDVDAYCIACSHLKPEETLVETEPFEILQFRKHNRAIIHHQTERTYKLDGVTVAKNRKKRMEQKTDSLEDWYVDMAKEHGKTQADAMRSRLTVKKSTRYYNTPGRMMPGTVFLYEGKRYVMTGQITNGKYYRAYGQEKRNFPAVKVRILTKNTGLVFVA

SEQ ID NO: 5 - protein, 598 aa, synthetic construct
MRYVYVLDVDGKPLMPTCRFGKVRRMLKSGQAKAVDTLPFTIQLTYKPRTRILQPVTLGQDPGRTHIGMAAVRFDGKELGRFHCITRNKEIPKLMADRMAARKASRRGERLARKRLARKLHTTAKHLNGRILPGCSKPIAVKDIINTESRFNNRILTKCKVCGKNTPLRRNVRELLLENIVRFLPLESELKETLKRTILEGQQGNINKLFRKLKFNQKDWPGKNLTDIAKNKLPGRLPFCKEHFAENEKFTTIEKSTFRLTPTATQLLRTHINLFRKLSGILPVTDVAVELNKFAFMQLDNPEMKKREIDFCHGPLCGTGGLEAAVKEQQDGKCLLCGKESIGHYHHIVPRSRRGSNTIANIAGLCPKCHELVHKDADTAESLTEMKTGLMKKYGGTSVLNQIIPKLVRTLAKLFPGHFHVTNGWNTKEFREKHHLEKDHDVDAYCIACSHLKPEETLVETEPFEILQFRKHNRAIIHHQTERTYKLDGVTVAKNRKKRMEQKTDSLEDWYVDMAKEHGKTQADAMRSRLTVKKSRRYYNTPGRMMPGTVFLYEGKRYVMTGQITNGKYYRAYGQEKRNFPAVKVRILTKNTGLVFVA

SEQ ID NO: 6 - protein, 657 aa, synthetic construct
MGSPKKKRKVGSMRYVYVLDVDGKPLMPTCRFGKVRRMLKSGQAKAVDTLPFTIQLTYKPRTRILQPVTLGQDPGRTNIGMAAVRFDGKELGRFHCITRNKEIPKLMADRMAARKASRRGERLARKRLARKLHTTAKHLNGRILPGCSEPIAVKDIINTESRFNNRILTKCKVCGKNTPLRRNVRELLLENIVRFLPLESELKETLKRTILEGQQGNINKLFRKLKFNQKDWPGKNLTDIAKNKLPGRLPFCKEHFAENEKFTTIEKSTFRLTPTATQLLRTHINLFRKLSGILPVTDVAVELNKFAFMQLDNPEMKKREIDFCHGPLCGTGGLEAAVKEQQDGKCLLCGKESIGHYHHIVPRSRRGSNTIANIAGLCPKCHELVHKDADTAESLTEMKTGLMKKYGGTSVLNQIIPKLVRTLAKLFPGHFHVTNGWNTKEFREKHHLEKDHDVDAYCIACSHLKPEETLVETEPFEILQFRKHNRAIIHHQTERTYKLDGVTVAKNRKKRMEQKTDSLEDWYVDMAKEHGKTQADAMRSRLTVKKSTRYYNTPGRMMPGTVFLYEGKRYVMTGQITNGKYYRAYGQEKRNFPAVKVRILTKNTGLVFVAGSKRPAATKKAGQAKKKKGSYPYDVPDYAYPYDVPDYAYPYDVPDYA

SEQ ID NO: 7 - protein, 657 aa, synthetic construct
MGSPKKKRKVGSMRYVYVLDVDGKPLMPTCRFGKVRRMLKSGQAKAVDTLPFTIQLTYKPRTRILQPVTLGQDPGRTHIGMAAVRFDGKELGRFHCITRNKEIPKLMADRMKARKASRRGERLARKRLARKLHTTAKHLNGRILPGCSKPIAVKDIINTESRFNNRILTKCKVCGKNTPLRRNVRELLLENIVRFLPLESELKETLKRTILEGQQGNINKLFRKLKFNQKDWPGKNLTDIAKNKLPGRLPFCKEHFAENEKFTTIEKSTFRLTPTATQLLRTHINLFRKLSGILPVTDVAVELNKFAFMQLDNPEMKKREIDFCHGPLCGTGGLEAAVKEQQDGKCLLCGKESIGHYHHIVPRSRRGSNTIANIAGLCPKCHELVHKDADTAESLTEMKTGLMKKYGGTSVLNQIIPKLVRTLAKLFPGHFHVTNGWNTKEFREKHHLEKDHDVDAYCIACSHLKPEETLVETEPFEILQFRKHNRAIIHHQTERTYKLDGVTVAKNRKKRMEQKTDSLEDWYVDMAKEHGKTQADAMRSRLTVKKSRRYYNTPGRMMPGTVFLYEGKRYVMTGQITNGKYYRAYGQEKRNFPAVKVRILTKNTGLVFVAGSKRPAATKKAGQAKKKKGSYPYDVPDYAYPYDVPDYAYPYDVPDYA

SEQ ID NO: 8 - protein, 610 aa, synthetic construct
MGSPKKKRKVGSMRYVYVLDVDGKPLMPTCRFGKVRRMLKSGQAKAVDTLPFTIQLTYKPRTRILQPVTLGQDPGRTNIGMAAVRFDGKELGRFHCITRNKEIPKLMADRMAARKASRRGERLARKRLARKLHTTAKHLNGRILPGCSEPIAVKDIINTESRFNNRILTKCKVCGKNTPLRRNVRELLLENIVRFLPLESELKETLKRTILEGQQGNINKLFRKLKFNQKDWPGKNLTDIAKNKLPGRLPFCKEHFAENEKFTTIEKSTFRLTPTATQLLRTHINLFRKLSGILPVTDVAVELNKFAFMQLDNPEMKKREIDFCHGPLCGTGGLEAAVKEQQDGKCLLCGKESIGHYHHIVPRSRRGSNTIANIAGLCPKCHELVHKDADTAESLTEMKTGLMKKYGGTSVLNQIIPKLVETLADLFPGHFHVTNGWNTKEFREKHHLEKDHDVDAYCIACSHLKPEETLVETEPFEILQFRKHNRAIIHHQTERTYKLDGVTVAKNRKKRMEQKTDSLEDWYVDMAKEHGKTQADAMRSRLTVIKSTRYYNTPGRMMPGTVFLYEGKRYVMTGQITNGKYYRAYGQEKRNFPAVKVRILTKNTGLVFVA

SEQ ID NO: 9 - protein, 105 aa, synthetic construct
ILTKCKVCGKNTPLRRNVRELLLENIVRFLPLESELKETLKRTILEGQQGNINKLFRKLKFNQKDWPGKNLTDIAKNKLPGRLPFCKEHFAENEKFTTIEKSTFR

SEQ ID NO: 10 - RNA, 133 nt, synthetic construct
  source 1..133: mol_type = other RNA
gtcaataacccgaaagggttactgagcagagcagtgacatgtcattcgccgcggggtgattccaagctccgcgctccggctagacatgcccatgctatggaaactttaacggtgaaaaccggcttacaacaaa

SEQ ID NO: 11 - protein, 6 aa, synthetic construct
QTSGGR

SEQ ID NO: 12 - protein, 6 aa, synthetic construct
RSSGGK

SEQ ID NO: 13 - protein, 6 aa, synthetic construct
QQSKGR

SEQ ID NO: 14 - protein, 6 aa, synthetic construct
QQGRGR

SEQ ID NO: 15 - protein, 6 aa, synthetic construct
QQNKGR

SEQ ID NO: 16 - protein, 6 aa, synthetic construct
QKDKGA

SEQ ID NO: 17 - protein, 6 aa, synthetic construct
QHCHGA

SEQ ID NO: 18 - protein, 6 aa, synthetic construct
NSHYGQ

SEQ ID NO: 19 - protein, 6 aa, synthetic construct
QHCHGN

SEQ ID NO: 20 - protein, 6 aa, synthetic construct
QQNKGD

SEQ ID NO: 21 - protein, 5 aa, synthetic construct
THAKS

SEQ ID NO: 22 - protein, 6 aa, synthetic construct
QKNNGV

SEQ ID NO: 23 - protein, 6 aa, synthetic construct
VLNKGA

SEQ ID NO: 24 - protein, 6 aa, synthetic construct
SSGAGK

SEQ ID NO: 25 - protein, 6 aa, synthetic construct
NSNGGA

SEQ ID NO: 26 - protein, 6 aa, synthetic construct
IKNRGR

SEQ ID NO: 27 - protein, 6 aa, synthetic construct
MHNKGK

SEQ ID NO: 28 - protein, 6 aa, synthetic construct
VFNYGK

SEQ ID NO: 29 - protein, 6 aa, synthetic construct
THNKGA

SEQ ID NO: 30 - protein, 6 aa, synthetic construct
AHGYGK

SEQ ID NO: 31 - protein, 6 aa, synthetic construct
NKNKGK

SEQ ID NO: 32 - protein, 6 aa, synthetic construct
THSYGK

SEQ ID NO: 33 - protein, 6 aa, synthetic construct
MHSYGR

SEQ ID NO: 34 - protein, 6 aa, synthetic construct
SQNKGT

SEQ ID NO: 35 - protein, 6 aa, synthetic construct
TTGLGK

SEQ ID NO: 36 - protein, 4 aa, synthetic construct
KTNN

SEQ ID NO: 37 - protein, 6 aa, synthetic construct
TQNKGR

SEQ ID NO: 38 - protein, 6 aa, synthetic construct
THCKGT

SEQ ID NO: 39 - protein, 6 aa, synthetic construct
QHTNGR

SEQ ID NO: 40 - protein, 6 aa, synthetic construct
QRTNGN

SEQ ID NO: 41 - protein, 6 aa, synthetic construct
QRTNGS

SEQ ID NO: 42 - protein, 6 aa, synthetic construct
QRTRGQ

SEQ ID NO: 43 - protein, 6 aa, synthetic construct
QASGGK

SEQ ID NO: 44 - protein, 6 aa, synthetic construct
QSSGGK

SEQ ID NO: 45 - protein, 6 aa, synthetic construct
QQSNGR

SEQ ID NO: 46 - protein, 6 aa, synthetic construct
QHSNGA

SEQ ID NO: 47 - protein, 6 aa, synthetic construct
QKNRGY

SEQ ID NO: 48 - protein, 6 aa, synthetic construct
QLSKGI

SEQ ID NO: 49 - protein, 6 aa, synthetic construct
TKNKGT

SEQ ID NO: 50 - protein, 6 aa, synthetic construct
GKNGYV

SEQ ID NO: 51 - protein, 12 aa, synthetic construct
MSGQHCHGAYLR

SEQ ID NO: 52 - protein, 12 aa, synthetic construct
LSGNSHYGQYYM

SEQ ID NO: 53 - protein, 12 aa, synthetic construct
MTGQHCHGNYFR

SEQ ID NO: 54 - protein, 12 aa, synthetic construct
MTGQQNKGDRYF

SEQ ID NO: 55 - protein, 12 aa, synthetic construct
KSGQLTNGQYFR

SEQ ID NO: 56 - protein, 12 aa, synthetic construct
LKGNACNGAYFH

SEQ ID NO: 57 - protein, 11 aa, synthetic construct
SSGTHAKSYFR

SEQ ID NO: 58 - protein, 12 aa, synthetic construct
LGGQKNNGVYFY

SEQ ID NO: 59 - protein, 12 aa, synthetic construct
VTGSSGAGKYVR

SEQ ID NO: 60 - protein, 12 aa, synthetic construct
VRSVHSYGKYVR

SEQ ID NO: 61 - protein, 12 aa, synthetic construct
VQGIKNRGRYLH

SEQ ID NO: 62 - protein, 12 aa, synthetic construct
LRGTSSYGKTVL

SEQ ID NO: 63 - protein, 12 aa, synthetic construct
VKGVFNYGKWVR

SEQ ID NO: 64 - protein, 10 aa, synthetic construct
VKGKHNDRWI

SEQ ID NO: 65 - protein, 12 aa, synthetic construct
VKGVQNRGKYIK

SEQ ID NO: 66 - protein, 12 aa, synthetic construct
IKGSHCNGSRLI

SEQ ID NO: 67 - protein, 12 aa, synthetic construct
SGGVMSYGRYIT

SEQ ID NO: 68 - protein, 12 aa, synthetic construct
SKGNKNKGKSIL

SEQ ID NO: 69 - protein, 12 aa, synthetic construct
AIGTHCKGKRVI

SEQ ID NO: 70 - protein, 12 aa, synthetic construct
VKGVFNRGTWVR

SEQ ID NO: 71 - protein, 12 aa, synthetic construct
CKGMHSYGRYVL

SEQ ID NO: 72 - protein, 12 aa, synthetic construct
IGGTQNKGRYAI

SEQ ID NO: 73 - protein, 12 aa, synthetic construct
TKGTHCKGTRIM

SEQ ID NO: 74 - protein, 401 aa, synthetic construct
MSTDATLIRTTPSHAEADATDTLVATPLMPPRRVISPWPGPGEGQSLMRIPVVDIRGMALMPCTPAKARHLLKSGNARPKRNKLGLFYVQLSYEQEPDNQSLVAGVDPGSKFEGLSVVGTKDTVLNLMVEAPDHVKGAVQTRRTMRRARRQRKWRRPKRFHNRLNRMQRIPPSTRSRWEAKARIVAHLRTILPFTDVVVEDVQAVTRKGKGGTWNGSFSPVQVGKEHLYRLLRAMGLTLHLREGWQTKELREQHGLKKTKSKSKQSFESHAVDSWVLAASISGAEHPTCTRLWYMVPAILHRRQLHRLQASKGGVRKPYGGTRSLGVKRGTLVEHKKYGRCTVGGVDRKRNTISLHEYRTNTRLTQAAKVETCRVLTWLSWRSWLLRGKRTSSKGKGSHSS

SEQ ID NO: 75 - protein, 432 aa, synthetic construct
MQPAKQQNWVFQINGDKQPLDMINPGRCRELQNRGKLASFRRFPYVVIQQQTIENPQTKEYILKIDPGSQWTGFAIQCGNDILFRAELNHRGEAIKFDLVKRAWFRRGRRSRNLRYRKKRLNRAKPEGWLAPSIRHRVLTVETWIKRFMRYCPIAWIEIEQVRFDTQKLANPEIDGVEYQQGELQGYEVREYLLQKWGRKCAYCGTENVPLEVEHIQSKSKGGSSRIGNLTLACHVCNVKKGNLDVRDFLAKSPDILNQVLENSTKPLKDAAAVNSTRYAIVKMAKSICENVKCSSGARTKMNRVRQGLEKTHSLDAACVGESGASIRVLTDRPLLITCKGHGSRQSIRVNASGFPAVKNAKTVFTHIAAGDVVRFTIGKDRKKAQAGTYTARVKTPTPKGFEVLIDGARISLSTMSNVVFVHRSDGYGYEL

SEQ ID NO: 76 - protein, 461 aa, synthetic construct
MAVFVIDKHKRPLMPCSEKRARLLLERGRAVVHRQVPFVIRLKDRTVQHSAVQPLRVALDPGSRATGMALVREKNTVDTGTGEVYRERIALNLFELVHRGHRIREQLDQRRNFRRRRRGANLRYRAPRFDNRRRPPGWLAPSLQHRVDTTMAWVRRLCRWAPASAIGIETVRFDTQRLQNPEISGVEYQQGALAGCEVREYLLEKWGRKCAYCGAENVPLEIEHIVPKSRGGSDRVSNLALACRACNQAKGNRDVRAFLADQPERLARILAQAKAPLKDAAAVNATRWALYRALVDTGLPVEAGTGGRTKWNRTRLGLPKTHALDALCVGQVDQVRHWRVPVLGIRCAGRGSYRRTRLTRHGFPRGYLTRNKSAFGFQTGDLIRAVVTKGKKAGTYLGRIAIRASGSFNIQTPMGVVQGIHHRFCTLLQRADGYGYFVQPKPTEAALSSPRLKAGVSSAGN

SEQ ID NO: 77 - protein, 439 aa, synthetic construct
MTTNVVFVIDTNQKPLQPCSAAVARKLLLRGKAAMFRRYPAVIILKKEVDSVGKPKIELRIDPGSKYTGFALVDSKDNADFIIWGTELEHRGAAICKELTKRSAIRRSRRNRKTRYRKKRFERRKPEGWLAPSLQHRVDTTLTWVKRICKFVPIMSISVEQVKFDLQKLENSDIQGIEYQQGTLAGYTLREALLEHWGRKCAYCDVENVFLEIEHIYPKSKGGSDKFSNLTLACHKCNINKGNKSIDEFLLSDHKRLEQIKLHQKKTLKDAAAVNATRKKLVTTLQEKTFLNVLVSDGASTKMTRLSSSLAKRHWIDAGCVNTTLIVILKTLQPLQVKCNGHGNKQFVTMDAYGFPRKSYEPKKVRKDWKAGDIIRVTKKDGTMLMGRVKKAAKKLVYIPFGGKEASFSSENAKAIHRSDGYRYSFAAIDSELLQKMAT

SEQ ID NO: 78 - protein, 419 aa, synthetic construct
MPNKYAFVLDSKGKLLDPTKSKKAWYLIRKGKASLVEEYPLIIKLKREVPKDQVNSDKLILGIDDGTKKVGFALVQKCQTKNKVLFKAVMEQRQDVSKKMEERRGYRRYRRSHKRYRPARFDNRSSSKRKGRIPPSILQKKQAILRVVNKLKKYIRIDKIVLEDVSIDIRKLTEGRELYNWEYQESNRLDENLRKATLYRDDCTCQLCGTTETMLHAHHIMPRRDGGADSIYNLITLCKACHKDKVDNNEYQYKDQFLAIIDSKELSDLKSASHVMQGKTWLRDKLSKIAQLEITSGGNTANKRIDYEIEKSHSNDAICTTGLLPVDNIDDIKEYYIKPLRKKSKAKIKELKCFRQRDLVKYTKRNGETYTGYITSLRIKNNKYNSKVCNFSTLKGKIFRGYGFRNLTLLNRPKGLMIV

SEQ ID NO: 79 - protein, 1409 aa, synthetic construct
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 80 - protein, 480 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAR

SEQ ID NO: 81 - protein, 1365 aa, synthetic construct
KYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 82 - protein, 7 aa, synthetic construct
PKKKRKV

SEQ ID NO: 83 - protein, 16 aa, synthetic construct
KRPAATKKAGQAKKKK

SEQ ID NO: 84 - protein, 9 aa, synthetic construct
PAAKRVKLD

SEQ ID NO: 85 - protein, 11 aa, synthetic construct
RQRRNELKRSP

SEQ ID NO: 86 - protein, 38 aa, synthetic construct
NQSSNFGPMKGGNFGGRSSGPYGGGGQYFAKPRNQGGY

SEQ ID NO: 87 - protein, 42 aa, synthetic construct
RMRIZFKNKGKDTAELRRRRVEVSVELRKAKKDEQILKRRNV

SEQ ID NO: 88 - protein, 8 aa, synthetic construct
VSRKRPRP

SEQ ID NO: 89 - protein, 8 aa, synthetic construct
PPKKARED

SEQ ID NO: 90 - protein, 8 aa, synthetic construct
PQPKKKPL

SEQ ID NO: 91 - protein, 12 aa, synthetic construct
SALIKKKKKMAP

SEQ ID NO: 92 - protein, 5 aa, synthetic construct
DRLRR

SEQ ID NO: 93 - protein, 7 aa, synthetic construct
PKQKKRK

SEQ ID NO: 94 - protein, 10 aa, synthetic construct
RKLKKKIKKL

SEQ ID NO: 95 - protein, 10 aa, synthetic construct
REKKKFLKRR

SEQ ID NO: 96 - protein, 20 aa, synthetic construct
KRKGDEVDGVDEVAKKKSKK

SEQ ID NO: 97 - protein, 17 aa, synthetic construct
RKCLQAGMNLEARKTKK

SEQ ID NO: 98 - protein, 5 aa, synthetic construct
GGGGS

SEQ ID NO: 99 - protein, 198 aa, Homo sapiens [sequence omitted]

SEQ ID NO: 100 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 101 - protein, 198 aa, Canis lupus
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 102 - protein, 199 aa, Bos taurus
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 103 - protein, 239 aa, Rattus sp.
MAVGSKPKAALVGPHWERERIWCFLCSTGLGTQQTGQTSRWLRPAATQDPVSPPRSLLMKQRKFLYHFKNVRWAKGRHETYLCYVVKRRDSATSFSLDFGYLRNKSGCHVELLFLRYISDWDLDPGRCYRVTWFTSWSPCYDCARHVADFLRGNPNLSLRIFTARLTGWGALPAGLMSPARPSDYFYCWNTFVENHERTFKAWEGLHENSVRLSRRLRRILLPLYEVDDLRDAFRTLGL

SEQ ID NO: 104 - protein, 429 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 105 - protein, 429 aa, Rattus sp.
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 106 - protein, 370 aa, Macaca mulatta
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 107 - protein, 384 aa, Pan troglodytes
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 108 - protein, 377 aa, Chlorocebus sabaeus
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 109 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 110 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 111 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 112 - protein, 396 aa, Rattus sp.
MQPQGLGPNAGMGPVCLGCSHRRPYSPIRNPLKKLYQQTFYFHFKNVRYAWGRKNNFLCYEVNGMDCALPVPLRQGVFRKQGHIHAELCFIYWFHDKVLRVLSPMEEFKVTWYMSWSPCSKCAEQVARFLAAHRNLSLAIFSSRLYYYLRNPNYQQKLCRLIQEGVHVAAMDLPEFKKCWNKFVDNDGQPFRPWMRLRINFSFYDCKLQEIFSRMNLLREDVFYLQFNNSHRVKPVQNRYYRRKSYLCYQLERANGQEPLKGYLLYKKGEQHVEILFLEKMRSMELSQVRITCYLTWSPCPNCARQLAAFKKDHPDLILRIYTSRLYFYWRKKFQKGLCTLWRSGIHVDVMDLPQFADCWTNFVNPQRPFRPWNELEKNSWRIQRRLRRIKESWGL

SEQ ID NO: 113 - protein, 227 aa, Bos taurus
DGWEVAFRSGTVLKAGVLGVSMTEGWAGSGHPGQGACVWTPGTRNTMNLLREVLFKQQFGNQPRVPAPYYRRKTYLCYQLKQRNDLTLDRGCFRNKKQRHAEIRFIDKINSLDLNPSQSYKIICYITWSPCPNCANELVNFITRNNHLKLEIFASRLYFHWIKSFKMGLQDLQNAGISVAVMTHTEFEDCWEQFVDNQSRPFQPWDKLEQYSASIRRRLQRILTAPI

SEQ ID NO: 114 - protein, 490 aa, Pan troglodytes
MNPQIRNPMEWMYQRTFYYNFENEPILYGRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRLSQAGARVKIMDDEEFAYCWENFVYNEGQPFMPWYKFDDNYAFLHRTLKEIIRHLMDPDTFTFNFNNDPLVLRRHQTYLCYEVERLDNGTWVLMDQHMGFLCNEAKNLLCGFYGRHAELRFLDLVPSLQLDPAQIYRVTWFISWSPCFSWGCAGQVRAFLQENTHVRLRIFAARIYDYDPLYKEALQMLRDAGAQVSIMTYDEFEYCWDTFVYRQGCPFQPWDGLEEHSQALSGRLRAILQVRASSLCMVPHRPPPPPQSPGPCLPLCSEPPLGSLLPTGRPAPSLPFLLTASFSFPPPASLPPLPSLSLSPGHLPVPSFHSLTSCSIQPPCSSRIRETEGWASVSKEGRDLG

SEQ ID NO: 115 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 116 - protein, 190 aa, Gorilla gorilla
MNPQIRNPMKAMYPGTFYFQFKNLWEANDRNETWLCFTVEGIKRRSVVSWKTGVFRNQVDSETHCHAERCFLSWFCDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 117 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 118 - protein, 202 aa, Macaca mulatta
MDGSPASRPRHLMDPNTFTFNFNNDLSVRGRHQTYLCYEVERLDNGTWVPMDERRGFLCNKAKNVPCGDYGCHVELRFLCEVPSWQLDPAQTYRVTWFISWSPCFRRGCAGQVRVFLQENKHVRLRIFAARIYDYDPLYQEALRTLRDAGAQVSIMTYEEFKHCWDTFVDRQGRPFQPWDGLDEHSQALSGRLRAILQNQGN

SEQ ID NO: 119 - protein, 185 aa, Bos taurus
MDEYTFTENFNNQGWPSKTYLCYEMERLDGDATIPLDEYKGFVRNKGLDQPEKPCHAELYFLGKIHSWNLDRNQHYRLTCFISWSPCYDCAQKLTTFLKENHHISLHILASRIYTHNRFGCHQSGLCELQAAGARITIMTFEDFKHCWETFVDHKGKPFQPWEGLNVKSQALCTELQAILKTQQN

SEQ ID NO: 120 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 121 - protein, 210 aa, Macaca mulatta
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 122 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 123 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 124 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 125 - protein, 229 aa, Rattus sp. [sequence omitted]

SEQ ID NO: 126 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 127 - protein, 224 aa, Mus musculus
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 128 - protein, 224 aa, Rattus sp.
MAQKEEAAEAAAPASQNGDDLENLEDPEKLKELIDLPPFEIVTGVRLPVNFFKFQFRNVEYSSGRNKTFLCYVVEAQSKGGQVQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYLWQNFVEQEEGESKAFEPWEDIQENFLYYEEKLADILK

SEQ ID NO: 129 - protein, 224 aa, Bos taurus
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 130 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQNQEN

SEQ ID NO: 131 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQ

SEQ ID NO: 132 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQ

SEQ ID NO: 133 - protein, 101 aa, Bacillus subtilis
EACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 134 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 135 - protein, 167 aa, Escherichia coli
MSEVEFSHEYWMRHALTLAKRAWDEREVPVGAVLVHNNRVIGEGWNRPIGRHDPTAHAAIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 136 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 137 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNAKTGAAGSLMDVLHYPGMNHRVEITEGILADECAALLCYFFRMPRQVFNAQKKAQSSTD

SEQ ID NO: 138 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRWAKTGAAGSLMDVLHYPGMNHRVEITEGILADECAALLCYFFRMPRQVFNAQKKAQSSTD

SEQ ID NO: 139 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRQAKTGAAGSLMDVLHYPGMNHRVEITEGILADECAALLCYFFRMPRQVFNAQKKAQSSTD

SEQ ID NO: 140 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGFRNAKTGAAGSLMDVLHYPGMNHRVEITEGILADECAALLCYFFRMPRQVFNAQKKAQSSTD

SEQ ID NO: 141 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGQRNAKTGAAGSLMDVLHYPGMNHRVEITEGILADECAALLCYFFRMPRQVFNAQKKAQSSTD

SEQ ID NO: 142 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGMRNAKTGAAGSLMDVLHYPGMNHRVEITEGILADECAALLCYFFRMPRQVFNAQKKAQSSTD

SEQ ID NO: 143 - protein, 167 aa, synthetic construct
MSEVEFSHEYWMRHALTLAKRARDEREVPVGAVLVLNNRVIGEGWNFAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 144 - protein, 167 aa, synthetic construct
MSEVEFSHEYWMRHALTLAKRARDEREVPVGAVLVLNNRVIGEGWNWAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 145 - protein, 167 aa, synthetic construct
MSEVEFSHEYWMRHALTLAKRARDEREVPVGAVLVLNNRVIGEGWNQAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 146 - protein, 167 aa, synthetic construct
MSEVEFSHEYWMRHALTLAKRARDEREVPVGAVLVLNNRVIGEGWNMAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 147 - protein, 167 aa, synthetic construct
MSEVEFSHEYWMRHALTLAKRAWDEREVPVGAVLVHNNRVIGEGWNRPIGRHDPTAHAQIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 148 - protein, 167 aa, Escherichia coli
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 149 - protein, 178 aa, Escherichia coli
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 150 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 151 - protein, 183 aa, Salmonella enterica
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 152 - protein, 164 aa, Shewanella putrefaciens
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 153 - protein, 173 aa, Haemophilus influenzae
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 154 - protein, 84 aa, unidentified
MTNLSDIIEKETGKQLVIQESILMLPEEVEEVIGNKPESDILVHTAYDESTDENVMLLTSDAPEYKPWALVIQDSNGENKIKML

SEQ ID NO: 155 - protein, 219 aa, Homo sapiens
KGHLTRLGLEFFDQPAVPLARAFLGQVLVRRLPNGTELRGRIVETQAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 156 - protein, 222 aa, synthetic construct
DLASLRAQQIELASSVIREDRLDKDPPDLIAGAAVGFEQGGEVTRAAMVLLKYPSLELVEYKVARIATTMPYIPGFLSFREYPALLAAWEMLSQKPDLVFVDGHGISHPRRLGVASHFGLLVDVPTIGVAKKRLCGKFEPLSSEPGALAPLMDKGEQLAWVWRSKARCNPLFIATGHRVSVDSALAWVQRCMKGYRLPEPTRWADAVASERPAFVRYTANQP

SEQ ID NO: 157 - protein, 181 aa, Erwinia tasmaniensis
MASRGVNKVILVGNLGQDPEVRYMPNGGAVANITLATSESWRDKQTGETKEKTEWHRVVLFGKLAEVAGEYLRKGSQVYIEGALQTRKWTDQAGVEKYTTEVVVNVGGTMQMLGGRSQGGGASAGGQNGGSNNGWGQPQQPQGGNQFSGGAQQQARPQQQPQQNNAPANNEPPIDFDDDIP

SEQ ID NO: 158 - protein, 209 aa, synthetic construct
MAGAQDFVPHTADLAELAAAAGECRGCGLYRDATQAVFGAGGRSARIMMIGEQPGDKEDLAGLPFVGPAGRLLDRALEAADIDRDALYVTNAVKHFKFTRAAGGKRRIHKTPSRTEVVACRPWLIAEMTSVEPDVVVLLGATAAKALLGNDFRVTQHRGEVLHVDDVPGDPALVATVHPSSLLRGPKEERESAFAGLVDDLRVAADVRP

SEQ ID NO: 159 - protein, 313 aa, synthetic construct
MIGQKTLYSFFSPSPARKRHAPSPEPAVQGTGVAGVPEESGDAAAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQEPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKEL

SEQ ID NO: 160 - protein, 60 aa, synthetic construct
  VARIANT 1..5: note = This region may encompass 3-5 residues
  VARIANT 7..11: note = This region may encompass 3-5 residues
  VARIANT 13..17: note = This region may encompass 3-5 residues
  VARIANT 19..23: note = This region may encompass 3-5 residues
  VARIANT 25..29: note = This region may encompass 3-5 residues
  VARIANT 31..35: note = This region may encompass 3-5 residues
  VARIANT 37..41: note = This region may encompass 3-5 residues
  VARIANT 43..47: note = This region may encompass 3-5 residues
  VARIANT 49..53: note = This region may encompass 3-5 residues
  VARIANT 55..59: note = This region may encompass 3-5 residues
  VARIANT 1..60: note = This sequence may encompass 1-10 GxS repeating units, wherein x is 3-5
GGGGGSGGGGGSGGGGGSGGGGGSGGGGGSGGGGGSGGGGGSGGGGGSGGGGGSGGGGGS

SEQ ID NO: 161 - protein, 51 aa, synthetic construct
  VARIANT 2..51: note = This region may encompass 1-10 GGGGS repeating units
SGGGGSGGGGSGGGGSGGGGSGGGGSGGGGSGGGGSGGGGSGGGGSGGGGS

SEQ ID NO: 162 - protein, 32 aa, synthetic construct
GGGGSGGGGSGGGGSGGGGSGGGGSGGGGSGG

SEQ ID NO: 163 - protein, 16 aa, synthetic construct
GSGGGSGGGGSGGGGS

SEQ ID NO: 164 - protein, 5 aa, synthetic construct
GGSGG

SEQ ID NO: 165 - protein, 6 aa, synthetic construct
HHHHHH

SEQ ID NO: 166 - DNA, 20 bp, unidentified
  source 1..20: mol_type = unassigned DNA
actacagtggtgcctggaaa

SEQ ID NO: 167 - DNA, 20 bp, unidentified
  source 1..20: mol_type = unassigned DNA
cccccttccctatgggaata

SEQ ID NO: 168 - DNA, 20 bp, unidentified
  source 1..20: mol_type = unassigned DNA
gtccaggcactgctcttgat

SEQ ID NO: 169 - DNA, 20 bp, unidentified
  source 1..20: mol_type = unassigned DNA
gagcaccccctgtcatccga

SEQ ID NO: 170 - DNA, 20 bp, unidentified
  source 1..20: mol_type = unassigned DNA
ttggtgttgtgacttcatta

SEQ ID NO: 171 - DNA, 20 bp, unidentified
  source 1..20: mol_type = unassigned DNA
gcaattataagccatttcac

SEQ ID NO: 172 - DNA, 20 bp, unidentified
  source 1..20: mol_type = unassigned DNA
aatcataattcatcatttag

SEQ ID NO: 173 - DNA, 20 bp, unidentified
  source 1..20: mol_type = unassigned DNA
actcatttttcaaaacacgc

SEQ ID NO: 174 - DNA, 20 bp, unidentified
  source 1..20: mol_type = unassigned DNA
tgatggttcccattagtcac

SEQ ID NO: 175 - DNA, 20 bp, unidentified
  source 1..20: mol_type = unassigned DNA
ttgcattattatttcagatg

SEQ ID NO: 176 - DNA, 20 bp, unidentified
  source 1..20: mol_type = unassigned DNA
aatgtggtcataagtaagaa

SEQ ID NO: 177 - DNA, 20 bp, unidentified
  source 1..20: mol_type = unassigned DNA
ggaaatacaatccctaaact

SEQ ID NO: 178 - DNA, 20 bp, unidentified
  source 1..20: mol_type = unassigned DNA
aatcttcctcaaccatgttc

SEQ ID NO: 179 - DNA, 20 bp, unidentified
  source 1..20: mol_type = unassigned DNA
actaatgacacaaacatgct

SEQ ID NO: 180 - DNA, 20 bp, unidentified
  source 1..20: mol_type = unassigned DNA
atttctaaagtacaaaacag

SEQ ID NO: 181 - DNA, 20 bp, unidentified
  source 1..20: mol_type = unassigned DNA
tctataggctcatagtgcaa

SEQ ID NO: 182 - DNA, 20 bp, unidentified
  source 1..20: mol_type = unassigned DNA
tgaactcatatctgtaagaa

SEQ ID NO: 183 - DNA, 20 bp, unidentified
  source 1..20: mol_type = unassigned DNA
ttctattctttcctgcatat

SEQ ID NO: 184 - DNA, 20 bp, unidentified
  source 1..20: mol_type = unassigned DNA
tcccttttaaatgcaaatca

SEQ ID NO: 185 - DNA, 20 bp, unidentified
  source 1..20: mol_type = unassigned DNA
aggatttttaaattgaattc

SEQ ID NO: 186 - DNA, 20 bp, unidentified
  source 1..20: mol_type = unassigned DNA
tttaagaaaaaagaagaggg

SEQ ID NO: 187 - DNA, 20 bp, unidentified
  source 1..20: mol_type = unassigned DNA
gtggggaatggcaagcaaaa

SEQ ID NO: 188 - DNA, 20 bp, unidentified
  source 1..20: mol_type = unassigned DNA
caggtcttagtaaaaaacaa

SEQ ID NO: 189 - DNA, 20 bp, unidentified
  source 1..20: mol_type = unassigned DNA
agggggccctgagccaggaa

SEQ ID NO: 190 - DNA, 20 bp, unidentified
  source 1..20: mol_type = unassigned DNA
aaacgtccctggacaggctg

SEQ ID NO: 191 - DNA, 20 bp, unidentified
  source 1..20: mol_type = unassigned DNA
ggggtcagcaaactttctct

SEQ ID NO: 192 - DNA, 20 bp, unidentified
  source 1..20: mol_type = unassigned DNA
actgaattggggttaaaatg

SEQ ID NO: 193 - DNA, 20 bp, unidentified
  source 1..20: mol_type = unassigned DNA
gctattgcttcaacttttct

SEQ ID NO: 194 - DNA, 20 bp, unidentified
  source 1..20: mol_type = unassigned DNA
ctactggccttatctcacag

SEQ ID NO: 195 - DNA, 20 bp, unidentified
  source 1..20: mol_type = unassigned DNA
ggctaatcttcttcccgtct

SEQ ID NO: 196 - DNA, 20 bp, unidentified
  source 1..20: mol_type = unassigned DNA
gcctgactctgcaaagccaa

SEQ ID NO: 197 - DNA, 20 bp, unidentified
  source 1..20: mol_type = unassigned DNA
tgtgcctctccctgcagacg

SEQ ID NO: 198 - DNA, 20 bp, unidentified
  source 1..20: mol_type = unassigned DNA
tcccctcatcaaaaatagga

SEQ ID NO: 199 - DNA, 20 bp, unidentified
  source 1..20: mol_type = unassigned DNA
gtccttaagagtacattgtt

SEQ ID NO: 200 - DNA, 20 bp, unidentified
  source 1..20: mol_type = unassigned DNA
acctgaaagagcaggtctta

SEQ ID NO: 201 - DNA, 20 bp, unidentified
  source 1..20: mol_type = unassigned DNA
acaaggtctttgtacagtta

SEQ ID NO: 202 - DNA, 20 bp, unidentified
  source 1..20: mol_type = unassigned DNA
caagccatttctccaaaagg

SEQ ID NO: 203 - DNA, 20 bp, unidentified
  source 1..20: mol_type = unassigned DNA
caaggggttggtaactgagg

SEQ ID NO: 204 - DNA, 20 bp, unidentified
  source 1..20: mol_type = unassigned DNA
caaggccgcacagctagtaa

SEQ ID NO: 205 - DNA, 20 bp, unidentified
  source 1..20: mol_type = unassigned DNA
acgtggcaaaaaaagatgga

SEQ ID NO: 206 - DNA, 20 bp, unidentified
  source 1..20: mol_type = unassigned DNA
ggctaatttttgtattttta

SEQ ID NO: 207 - DNA, 20 bp, unidentified
  source 1..20: mol_type = unassigned DNA
aactaatagtgttcttatat

SEQ ID NO: 208 - DNA, 20 bp, unidentified
  source 1..20: mol_type = unassigned DNA
gagtcctattgacatcgcca

SEQ ID NO: 209 - DNA, 20 bp, unidentified
  source 1..20: mol_type = unassigned DNA
ggcggattgttgtttaactt

SEQ ID NO: 210 - DNA, 20 bp, unidentified
  source 1..20: mol_type = unassigned DNA
agccgtgttattttagcata

SEQ ID NO: 211 - DNA, 20 bp, unidentified
  source 1..20: mol_type = unassigned DNA
aacagaagtaaaaatgtttt

SEQ ID NO: 212 - DNA, 20 bp, unidentified
  source 1..20: mol_type = unassigned DNA
gatcaaatgagataacagaa

SEQ ID NO: 213 - DNA, 20 bp, unidentified
  source 1..20: mol_type = unassigned DNA
tagcaacccaccacttgtgg

SEQ ID NO: 214 - DNA, 20 bp, unidentified
  source 1..20: mol_type = unassigned DNA
ggacctgcacttcttcatct

SEQ ID NO: 215 - DNA, 26 bp, synthetic construct
  source 1..26: mol_type = other DNA
cactatgagcctatagactatcagtt

SEQ ID NO: 216 - DNA, 28 bp, synthetic construct
  source 1..28: mol_type = other DNA
gaaaattgactgcttcttacttttctaa

SEQ ID NO: 217 - DNA, 20 bp, unidentified
  source 1..20: mol_type = unassigned DNA
gccatgacaactgggtggaa

SEQ ID NO: 218 - DNA, 18 bp, synthetic construct
  source 1..18: mol_type = other DNA
ctcacctctccaaagctg

SEQ ID NO: 219 - DNA, 17 bp, synthetic construct
  source 1..17: mol_type = other DNA
gaaaggcccattgctct

SEQ ID NO: 220 - DNA, 20 bp, unidentified
  source 1..20: mol_type = unassigned DNA
ggagtggggcagaatctgga

SEQ ID NO: 221 - DNA, 20 bp, unidentified
  source 1..20: mol_type = unassigned DNA
ttgttttctgtagggagaag

SEQ ID NO: 222 - DNA, 20 bp, unidentified
  source 1..20: mol_type = unassigned DNA
taccagccactgggtggctt

SEQ ID NO: 223 - DNA, 20 bp, unidentified
  source 1..20: mol_type = unassigned DNA
ctatccagcagtgagaacaa

SEQ ID NO: 224 - DNA, 20 bp, unidentified
  source 1..20: mol_type = unassigned DNA
caaacctcacaaccgtgagc

SEQ ID NO: 225 - DNA, 20 bp, unidentified
  source 1..20: mol_type = unassigned DNA
gaaataaccaagaaaaaagc

SEQ ID NO: 226 - DNA, 20 bp, unidentified
  source 1..20: mol_type = unassigned DNA
tgaagactgagtccatttac

SEQ ID NO: 227 - DNA, 25 bp, unidentified
  source 1..25: mol_type = unassigned DNA
ggccaggttgtaatcctagccttat

SEQ ID NO: 228 - DNA, 12 bp, synthetic construct
  source 1..12: mol_type = other DNA
tcctagccttat

SEQ ID NO: 229 - DNA, 13 bp, synthetic construct
  source 1..13: mol_type = other DNA
gtcctagccttat

SEQ ID NO: 230 - DNA, 13 bp, synthetic construct
  source 1..13: mol_type = other DNA
atcctagccttat

SEQ ID NO: 231 - DNA, 14 bp, synthetic construct
  source 1..14: mol_type = other DNA
gatcctagccttat

SEQ ID NO: 232 - DNA, 14 bp, synthetic construct
  source 1..14: mol_type = other DNA
aatcctagccttat

SEQ ID NO: 233 - DNA, 15 bp, synthetic construct
  source 1..15: mol_type = other DNA
gaatcctagccttat

SEQ ID NO: 234 - DNA, 15 bp, synthetic construct
  source 1..15: mol_type = other DNA
taatcctagccttat

SEQ ID NO: 235 - DNA, 16 bp, synthetic construct
  source 1..16: mol_type = other DNA
gtaatcctagccttat

SEQ ID NO: 236 - DNA, 17 bp, synthetic construct
  source 1..17: mol_type = other DNA
tgtaatcctagccttat

SEQ ID NO: 237 - DNA, 18 bp, synthetic construct
  source 1..18: mol_type = other DNA
gtgtaatcctagccttat

SEQ ID NO: 238 - DNA, 18 bp, synthetic construct
  source 1..18: mol_type = other DNA
ttgtaatcctagccttat

SEQ ID NO: 239 - DNA, 19 bp, synthetic construct
  source 1..19: mol_type = other DNA
gttgtaatcctagccttat

SEQ ID NO: 240 - DNA, 20 bp, synthetic construct
  source 1..20: mol_type = other DNA
ggttgtaatcctagccttat

SEQ ID NO: 241 - DNA, 21 bp, synthetic construct
  source 1..21: mol_type = other DNA
aggttgtaatcctagccttat

SEQ ID NO: 242 - DNA, 22 bp, synthetic construct
  source 1..22: mol_type = other DNA
gaggttgtaatcctagccttat

SEQ ID NO: 243 - DNA, 22 bp, synthetic construct
  source 1..22: mol_type = other DNA
caggttgtaatcctagccttat

SEQ ID NO: 244 - DNA, 23 bp, synthetic construct
  source 1..23: mol_type = other DNA
gcaggttgtaatcctagccttat

SEQ ID NO: 245 - DNA, 23 bp, synthetic construct
  source 1..23: mol_type = other DNA
ccaggttgtaatcctagccttat

SEQ ID NO: 246 - DNA, 24 bp, synthetic construct
  source 1..24: mol_type = other DNA
gccaggttgtaatcctagccttat

SEQ ID NO: 247 - DNA, 25 bp, unidentified
  source 1..25: mol_type = unassigned DNA
ctatcgccatgacaactgggtggaa

SEQ ID NO: 248 - DNA, 12 bp, synthetic construct
  source 1..12: mol_type = other DNA
aactgggtggaa

SEQ ID NO: 249 - DNA, 13 bp, synthetic construct
  source 1..13: mol_type = other DNA
gaactgggtggaa

SEQ ID NO: 250 - DNA, 13 bp, synthetic construct
  source 1..13: mol_type = other DNA
caactgggtggaa

SEQ ID NO: 251 - DNA, 14 bp, synthetic construct
  source 1..14: mol_type = other DNA
gcaactgggtggaa

SEQ ID NO: 252 - DNA, 14 bp, synthetic construct
  source 1..14: mol_type = other DNA
acaactgggtggaa

SEQ ID NO: 253 - DNA, 15 bp, synthetic construct
  source 1..15: mol_type = other DNA
gacaactgggtggaa

SEQ ID NO: 254 - DNA, 16 bp, synthetic construct
  source 1..16: mol_type = other DNA
tgacaactgggtggaa

SEQ ID NO: 255 - DNA, 17 bp, synthetic construct
  source 1..17: mol_type = other DNA
gtgacaactgggtggaa

SEQ ID NO: 256 - DNA, 17 bp, synthetic construct
  source 1..17: mol_type = other DNA
atgacaactgggtggaa

SEQ ID NO: 257 - DNA, 18 bp, synthetic construct
  source 1..18: mol_type = other DNA
gatgacaactgggtggaa

SEQ ID NO: 258 - DNA, 18 bp, synthetic construct
  source 1..18: mol_type = other DNA
catgacaactgggtggaa

SEQ ID NO: 259 - DNA, 19 bp, synthetic construct
  source 1..19: mol_type = other DNA
gcatgacaactgggtggaa

SEQ ID NO: 260 - DNA, 19 bp, synthetic construct
  source 1..19: mol_type = other DNA
ccatgacaactgggtggaa

SEQ ID NO: 261 - DNA, 21 bp, synthetic construct
  source 1..21: mol_type = other DNA
cgccatgacaactgggtggaa

SEQ ID NO: 262 - DNA, 22 bp, synthetic construct
  source 1..22: mol_type = other DNA
gcgccatgacaactgggtggaa

SEQ ID NO: 263 - DNA, 22 bp, synthetic construct
  source 1..22: mol_type = other DNA
tcgccatgacaactgggtggaa

SEQ ID NO: 264 - DNA, 23 bp, synthetic construct
  source 1..23: mol_type = other DNA
gtcgccatgacaactgggtggaa

SEQ ID NO: 265 - DNA, 23 bp, synthetic construct
  source 1..23: mol_type = other DNA
atcgccatgacaactgggtggaa

SEQ ID NO: 266 - DNA, 24 bp, synthetic construct
  source 1..24: mol_type = other DNA
gatcgccatgacaactgggtggaa

SEQ ID NO: 267 - DNA, 24 bp, synthetic construct
  source 1..24: mol_type = other DNA
tatcgccatgacaactgggtggaa

SEQ ID NO: 268 - DNA, 25 bp, synthetic construct
  source 1..25: mol_type = other DNA
gtatcgccatgacaactgggtggaa

SEQ ID NO: 269 - DNA, 26 bp, synthetic construct
  source 1..26: mol_type = other DNA
gctatcgccatgacaactgggtggaa

SEQ ID NO: 270 - DNA, 12 bp, synthetic construct
  source 1..12: mol_type = other DNA
aatccctaaact

SEQ ID NO: 271 - DNA, 13 bp, synthetic construct
  source 1..13: mol_type = other DNA
gaatccctaaact

SEQ ID NO: 272 - DNA, 13 bp, synthetic construct
  source 1..13: mol_type = other DNA
caatccctaaact

SEQ ID NO: 273 - DNA, 14 bp, synthetic construct
  source 1..14: mol_type = other DNA
gcaatccctaaact

SEQ ID NO: 274 - DNA, 14 bp, synthetic construct
  source 1..14: mol_type = other DNA
acaatccctaaact

SEQ ID NO: 275 - DNA, 15 bp, synthetic construct
  source 1..15: mol_type = other DNA
gacaatccctaaact

SEQ ID NO: 276 - DNA, 15 bp, synthetic construct
  source 1..15: mol_type = other DNA
tacaatccctaaact

SEQ ID NO: 277 - DNA, 16 bp, synthetic construct
  source 1..16: mol_type = other DNA
gtacaatccctaaact

SEQ ID NO: 278 - DNA, 16 bp, synthetic construct
  source 1..16: mol_type = other DNA
atacaatccctaaact

SEQ ID NO: 279 - DNA, 17 bp, synthetic construct
  source 1..17: mol_type = other DNA
gatacaatccctaaact

SEQ ID NO: 280 - DNA, 17 bp, synthetic construct
  source 1..17: mol_type = other DNA
aatacaatccctaaact

SEQ ID NO: 281 - DNA, 18 bp, synthetic construct
  source 1..18: mol_type = other DNA
gaatacaatccctaaact

SEQ ID NO: 282 - DNA, 18 bp, synthetic construct
  source 1..18: mol_type = other DNA
aaatacaatccctaaact

SEQ ID NO: 283 - DNA, 19 bp, synthetic construct
  source 1..19: mol_type = other DNA
gaaatacaatccctaaact

SEQ ID NO: 284 - DNA, 12 bp, synthetic construct
  source 1..12: mol_type = other DNA
tatctgtaagaa

SEQ ID NO: 285 - DNA, 13 bp, synthetic construct
  source 1..13: mol_type = other DNA
gtatctgtaagaa

SEQ ID NO: 286 - DNA, 13 bp, synthetic construct
  source 1..13: mol_type = other DNA
atatctgtaagaa

SEQ ID NO: 287 - DNA, 14 bp, synthetic construct
  source 1..14: mol_type = other DNA
gatatctgtaagaa

SEQ ID NO: 288 - DNA, 14 bp, synthetic construct
  source 1..14: mol_type = other DNA
catatctgtaagaa

SEQ ID NO: 289 - DNA, 15 bp, synthetic construct
  source 1..15: mol_type = other DNA
gcatatctgtaagaa

SEQ ID NO: 290 - DNA, 15 bp, synthetic construct
  source 1..15: mol_type = other DNA
tcatatctgtaagaa

SEQ ID NO: 291 - DNA, 16 bp, synthetic construct
  source 1..16: mol_type = other DNA
gtcatatctgtaagaa

SEQ ID NO: 292 - DNA, 16 bp, synthetic construct
  source 1..16: mol_type = other DNA
ctcatatctgtaagaa

SEQ ID NO: 293 - DNA, 17 bp, synthetic construct
  source 1..17: mol_type = other DNA
gctcatatctgtaagaa

SEQ ID NO: 294 - DNA, 17 bp, synthetic construct
  source 1..17: mol_type = other DNA
actcatatctgtaagaa

SEQ ID NO: 295 - DNA, 18 bp, synthetic construct
  source 1..18: mol_type = other DNA
gactcatatctgtaagaa

SEQ ID NO: 296 - DNA, 18 bp, synthetic construct
  source 1..18: mol_type = other DNA
aactcatatctgtaagaa

SEQ ID NO: 297 - DNA, 19 bp, synthetic construct
  source 1..19: mol_type = other DNA
gaactcatatctgtaagaa

SEQ ID NO: 298 - DNA, 21 bp, synthetic construct
  source 1..21: mol_type = other DNA
gtgaactcatatctgtaagaa

SEQ ID NO: 299 - DNA, 225 bp, synthetic construct
  source 1..225: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 300 - DNA, 184 bp, synthetic construct
  source 1..184: mol_type = other DNA
  misc_difference 1..20: note = a, c, t, g, unknown or other
nnnnnnnnnnnnnnnnnnnngtcaataacccgaaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 301 - DNA, 188 bp, synthetic construct
  source 1..188: mol_type = other DNA
  misc_difference 1..20: note = a, c, t, g, unknown or other
nnnnnnnnnnnnnnnnnnnngtcaataacccatgaaaaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 302 - DNA, 191 bp, synthetic construct
  source 1..191: mol_type = other DNA
  misc_difference 1..20: note = a, c, t, g, unknown or other
nnnnnnnnnnnnnnnnnnnngtcaataacccatgagaaaaaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 303 - DNA, 195 bp, synthetic construct
  source 1..195: mol_type = other DNA
  misc_difference 1..20: note = a, c, t, g, unknown or other
nnnnnnnnnnnnnnnnnnnngtcaataacccatgactgaaaaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 304 - DNA, 199 bp, synthetic construct
  source 1..199: mol_type = other DNA
  misc_difference 1..20: note = a, c, t, g, unknown or other
nnnnnnnnnnnnnnnnnnnngtcaataacccatgactgagaaaagaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 305 - DNA, 203 bp, synthetic construct
  source 1..203: mol_type = other DNA
  misc_difference 1..20: note = a, c, t, g, unknown or other
nnnnnnnnnnnnnnnnnnnngtcaataacccatgactgaaggaaaatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 306 - DNA, 207 bp, synthetic construct
  source 1..207: mol_type = other DNA
  misc_difference 1..20: note = a, c, t, g, unknown or other
nnnnnnnnnnnnnnnnnnnngtcaataacccatgactgaagtcgaaaagatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 307 - DNA, 210 bp, synthetic construct
  source 1..210: mol_type = other DNA
  misc_difference 1..20: note = a, c, t, g, unknown or other
nnnnnnnnnnnnnnnnnnnngtcaataacccatgactgaagtcatgaaaatgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 308 - DNA, 214 bp, synthetic construct
  source 1..214: mol_type = other DNA
  misc_difference 1..20: note = a, c, t, g, unknown or other
nnnnnnnnnnnnnnnnnnnngtcaataacccatgactgaagtcatgggaaaacctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 309 - DNA, 218 bp, synthetic construct
  source 1..218: mol_type = other DNA
  misc_difference 1..20: note = a, c, t, g, unknown or other
nnnnnnnnnnnnnnnnnnnngtcaataacccatgactgaagtcatgggcgaaaagtcctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 310 - DNA, 222 bp, synthetic construct
  source 1..222: mol_type = other DNA
  misc_difference 1..20: note = a, c, t, g, unknown or other
nnnnnnnnnnnnnnnnnnnngtcaataacccatgactgaagtcatgggcttgaaaaaggtcctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 311 - DNA, 183 bp, synthetic construct
  source 1..183: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 312 - DNA, 187 bp, synthetic construct
  source 1..187: mol_type = other DNA
  misc_difference 1..20: note = a, c, t, g, unknown or other
nnnnnnnnnnnnnnnnnnnngtcaataacccatgaaaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 313 - DNA, 190 bp, synthetic construct
  source 1..190: mol_type = other DNA
  misc_difference 1..20: note = a, c, t, g, unknown or other
nnnnnnnnnnnnnnnnnnnngtcaataacccatgagaaaaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 314 - DNA, 194 bp, synthetic construct
  source 1..194: mol_type = other DNA
  misc_difference 1..20: note = a, c, t, g, unknown or other
nnnnnnnnnnnnnnnnnnnngtcaataacccatgactgaaaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 315 - DNA, 198 bp, synthetic construct
  source 1..198: mol_type = other DNA
  misc_difference 1..20: note = a, c, t, g, unknown or other
nnnnnnnnnnnnnnnnnnnngtcaataacccatgactgagaaagaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 316 - DNA, 202 bp, synthetic construct
  source 1..202: mol_type = other DNA
  misc_difference 1..20: note = a, c, t, g, unknown or other
nnnnnnnnnnnnnnnnnnnngtcaataacccatgactgaaggaaatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 317 - DNA, 206 bp, synthetic construct
  source 1..206: mol_type = other DNA
  misc_difference 1..20: note = a, c, t, g, unknown or other
nnnnnnnnnnnnnnnnnnnngtcaataacccatgactgaagtcgaaagatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 318 - DNA, 209 bp, synthetic construct
  source 1..209: mol_type = other DNA
  misc_difference 1..20: note = a, c, t, g, unknown or other
nnnnnnnnnnnnnnnnnnnngtcaataacccatgactgaagtcatgaaatgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 319 - DNA, 213 bp, synthetic construct
  source 1..213: mol_type = other DNA
  misc_difference 1..20: note = a, c, t, g, unknown or other
nnnnnnnnnnnnnnnnnnnngtcaataacccatgactgaagtcatgggaaacctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 320 - DNA, 217 bp, synthetic construct
  source 1..217: mol_type = other DNA
  misc_difference 1..20: note = a, c, t, g, unknown or other
nnnnnnnnnnnnnnnnnnnngtcaataacccatgactgaagtcatgggcgaaagtcctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 321 - DNA, 221 bp, synthetic construct
  source 1..221: mol_type = other DNA
  misc_difference 1..20: note = a, c, t, g, unknown or other
nnnnnnnnnnnnnnnnnnnngtcaataacccatgactgaagtcatgggcttgaaaaggtcctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 322 - DNA, 223 bp, synthetic construct
  source 1..223: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagttatt

SEQ ID NO: 323 - DNA, 221 bp, synthetic construct
  source 1..221: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagtta

SEQ ID NO: 324 - DNA, 219 bp, synthetic construct
  source 1..219: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataaggagt

SEQ ID NO: 325 - DNA, 217 bp, synthetic construct
  source 1..217: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataagga

SEQ ID NO: 326 - DNA, 215 bp, synthetic construct
  source 1..215: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaataag

SEQ ID NO: 327 - DNA, 213 bp, synthetic construct
  source 1..213: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaata

SEQ ID NO: 328 - DNA, 211 bp, synthetic construct
  source 1..211: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggcttacaacaaa

SEQ ID NO: 329 - DNA, 209 bp, synthetic construct
  source 1..209: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggcttacaaca

SEQ ID NO: 330 - DNA, 207 bp, synthetic construct
  source 1..207: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggcttacaa

SEQ ID NO: 331 - DNA, 205 bp, synthetic construct
  source 1..205: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggcttac

SEQ ID NO: 332 - DNA, 203 bp, synthetic construct
  source 1..203: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggctt

SEQ ID NO: 333 - DNA, 201 bp, synthetic construct
  source 1..201: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggttttccgctcataccggc

SEQ ID NO: 334 - DNA, 215 bp, synthetic construct
  source 1..215: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatggaaacataccggcttacaacaaataaggagttattag

SEQ ID NO: 335 - DNA, 217 bp, synthetic construct
  source 1..217: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgaaatcataccggcttacaacaaataaggagttattag

SEQ ID NO: 336 - DNA, 219 bp, synthetic construct
  source 1..219: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtggaaactcataccggcttacaacaaataaggagttattag

SEQ ID NO: 337 - DNA, 223 bp, synthetic construct
  source 1..223: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgtgcggaaacgctcataccggcttacaacaaataaggagttattag

SEQ ID NO: 338 - DNA, 213 bp, synthetic construct
  source 1..213: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtatgaaaataccggcttacaacaaataaggagttattag

SEQ ID NO: 339 - DNA, 209 bp, synthetic construct
  source 1..209: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggtgaaaaccggcttacaacaaataaggagttattag

SEQ ID NO: 340 - DNA, 205 bp, synthetic construct
  source 1..205: mol_type = other DNA
  misc_difference 1..20: note = a, c, t, g, unknown or other
nnnnnnnnnnnnnnnnnnnngtcaataacccatgactgaagtcatgggcttgcagatgcaggtcctgatggaagaaagggttactgagcagagcagtgacatgtcattcgccgcggggtgattccaagctccgcgctccggctagacatgcccatgctatggaaactttaacggaaacggcttacaacaaataaggagttattag

SEQ ID NO: 341 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccggcttacaacaaa

SEQ ID NO: 342 - DNA, 146 bp, synthetic construct
  source 1..146: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccggctta

SEQ ID NO: 343 - DNA, 147 bp, synthetic construct
  source 1..147: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccggcttac

SEQ ID NO: 344 - DNA, 152 bp, synthetic construct
  source 1..152: mol_type = other DNA
  misc_difference 1..20: note = a, c, t, g, unknown or other
nnnnnnnnnnnnnnnnnnnntcaataacccgaaagggttactgagcagagcagtgacatgtcattcgccgcggggtgattccaagctccgcgctccggctagacatgcccatgctatggaaactttaacggtgaaaaccggcttacaacaaa

SEQ ID NO: 345 - DNA, 151 bp, synthetic construct
  source 1..151: mol_type = other DNA
  misc_difference 1..20: note = a, c, t, g, unknown or other
nnnnnnnnnnnnnnnnnnnngtcaataaccgaaaggttactgagcagagcagtgacatgtcattcgccgcggggtgattccaagctccgcgctccggctagacatgcccatgctatggaaactttaacggtgaaaaccggcttacaacaaa

SEQ ID NO: 346 - DNA, 149 bp, synthetic construct
  source 1..149: mol_type = other DNA
  misc_difference 1..20: note = a, c, t, g, unknown or other
nnnnnnnnnnnnnnnnnnnngtcaataacgaaagttactgagcagagcagtgacatgtcattcgccgcggggtgattccaagctccgcgctccggctagacatgcccatgctatggaaactttaacggtgaaaaccggcttacaacaaa

SEQ ID NO: 347 - DNA, 147 bp, synthetic construct
  source 1..147: mol_type = other DNA
  misc_difference 1..20: note = a, c, t, g, unknown or other
nnnnnnnnnnnnnnnnnnnngtcaataagaaattactgagcagagcagtgacatgtcattcgccgcggggtgattccaagctccgcgctccggctagacatgcccatgctatggaaactttaacggtgaaaaccggcttacaacaaa

SEQ ID NO: 348 - DNA, 145 bp, synthetic construct
  source 1..145: mol_type = other DNA
  misc_difference 1..20: note = a, c, t, g, unknown or other
nnnnnnnnnnnnnnnnnnnngtcaatagaaatactgagcagagcagtgacatgtcattcgccgcggggtgattccaagctccgcgctccggctagacatgcccatgctatggaaactttaacggtgaaaaccggcttacaacaaa

SEQ ID NO: 349 - DNA, 143 bp, synthetic construct
  source 1..143: mol_type = other DNA
  misc_difference 1..20: note = a, c, t, g, unknown or other
nnnnnnnnnnnnnnnnnnnngtcaatgaaaactgagcagagcagtgacatgtcattcgccgcggggtgattccaagctccgcgctccggctagacatgcccatgctatggaaactttaacggtgaaaaccggcttacaacaaa

SEQ ID NO: 350 - DNA, 141 bp, synthetic construct
  source 1..141: mol_type = other DNA
  misc_difference 1..20: note = a, c, t, g, unknown or other
nnnnnnnnnnnnnnnnnnnngtcaagaaactgagcagagcagtgacatgtcattcgccgcggggtgattccaagctccgcgctccggctagacatgcccatgctatggaaactttaacggtgaaaaccggcttacaacaaa

SEQ ID NO: 351 - DNA, 139 bp, synthetic construct
  source 1..139: mol_type = other DNA
  misc_difference 1..20: note = a, c, t, g, unknown or other
nnnnnnnnnnnnnnnnnnnngtcagaaatgagcagagcagtgacatgtcattcgccgcggggtgattccaagctccgcgctccggctagacatgcccatgctatggaaactttaacggtgaaaaccggcttacaacaaa

SEQ ID NO: 352 - DNA, 137 bp, synthetic construct
  source 1..137: mol_type = other DNA
  misc_difference 1..20: note = a, c, t, g, unknown or other
nnnnnnnnnnnnnnnnnnnngtcgaaagagcagagcagtgacatgtcattcgccgcggggtgattccaagctccgcgctccggctagacatgcccatgctatggaaactttaacggtgaaaaccggcttacaacaaa

SEQ ID NO: 353 - DNA, 135 bp, synthetic construct
  source 1..135: mol_type = other DNA
  misc_difference 1..20: note = a, c, t, g, unknown or other
nnnnnnnnnnnnnnnnnnnngtgaaaagcagagcagtgacatgtcattcgccgcggggtgattccaagctccgcgctccggctagacatgcccatgctatggaaactttaacggtgaaaaccggcttacaacaaa

SEQ ID NO: 354 - DNA, 133 bp, synthetic construct
  source 1..133: mol_type = other DNA
  misc_difference 1..20: note = a, c, t, g, unknown or other
nnnnnnnnnnnnnnnnnnnnggaaagcagagcagtgacatgtcattcgccgcggggtgattccaagctccgcgctccggctagacatgcccatgctatggaaactttaacggtgaaaaccggcttacaacaaa

SEQ ID NO: 355 - DNA, 151 bp, synthetic construct
  source 1..151: mol_type = other DNA
  misc_difference 1..20: note = a, c, t, g, unknown or other
nnnnnnnnnnnnnnnnnnnngtaataacccgaaagggttactagcagagcagtgacatgtcattcgccgcggggtgattccaagctccgcgctccggctagacatgcccatgctatggaaactttaacggtgaaaaccggcttacaacaaa

SEQ ID NO: 356 - DNA, 149 bp, synthetic construct
  source 1..149: mol_type = other DNA
  misc_difference 1..20: note = a, c, t, g, unknown or other
nnnnnnnnnnnnnnnnnnnngtataacccgaaagggttacagcagagcagtgacatgtcattcgccgcggggtgattccaagctccgcgctccggctagacatgcccatgctatggaaactttaacggtgaaaaccggcttacaacaaa

SEQ ID NO: 357 - DNA, 147 bp, synthetic construct
  source 1..147: mol_type = other DNA
  misc_difference 1..20: note = a, c, t, g, unknown or other
nnnnnnnnnnnnnnnnnnnngttaacccgaaagggttaagcagagcagtgacatgtcattcgccgcggggtgattccaagctccgcgctccggctagacatgcccatgctatggaaactttaacggtgaaaaccggcttacaacaaa

SEQ ID NO: 358 - DNA, 145 bp, synthetic construct
  source 1..145: mol_type = other DNA
  misc_difference 1..20: note = a, c, t, g, unknown or other
nnnnnnnnnnnnnnnnnnnngtaacccgaaagggttagcagagcagtgacatgtcattcgccgcggggtgattccaagctccgcgctccggctagacatgcccatgctatggaaactttaacggtgaaaaccggcttacaacaaa

SEQ ID NO: 359 - DNA, 143 bp, synthetic construct
  source 1..143: mol_type = other DNA
  misc_difference 1..20: note = a, c, t, g, unknown or other
nnnnnnnnnnnnnnnnnnnngtacccgaaagggtagcagagcagtgacatgtcattcgccgcggggtgattccaagctccgcgctccggctagacatgcccatgctatggaaactttaacggtgaaaaccggcttacaacaaa

SEQ ID NO: 360 - DNA, 141 bp, synthetic construct
  source 1..141: mol_type = other DNA
  misc_difference 1..20: note = a, c, t, g, unknown or other
nnnnnnnnnnnnnnnnnnnngtcccgaaagggagcagagcagtgacatgtcattcgccgcggggtgattccaagctccgcgctccggctagacatgcccatgctatggaaactttaacggtgaaaaccggcttacaacaaa

SEQ ID NO: 361 - DNA, 139 bp, synthetic construct
  source 1..139: mol_type = other DNA
  misc_difference 1..20: note = a, c, t, g, unknown or other
nnnnnnnnnnnnnnnnnnnngtccgaaaggagcagagcagtgacatgtcattcgccgcggggtgattccaagctccgcgctccggctagacatgcccatgctatggaaactttaacggtgaaaaccggcttacaacaaa

SEQ ID NO: 362 - DNA, 153 bp, synthetic construct
  source 1..153: mol_type = other DNA
  misc_difference 1..20: note = a, c, t, g, unknown or other
nnnnnnnnnnnnnnnnnnnngtcagtaacccgaaagggttactgagcagagcagtgacatgtcattcgccgcggggtgattccaagctccgcgctccggctagacatgcccatgctatggaaactttaacggtgaaaaccggcttacaacaaa

SEQ ID NO: 363 - DNA, 153 bp, synthetic construct
  source 1..153: mol_type = other DNA
  misc_difference 1..20: note = a, c, t, g, unknown or other
nnnnnnnnnnnnnnnnnnnngtcactaacccgaaagggttagtgagcagagcagtgacatgtcattcgccgcggggtgattccaagctccgcgctccggctagacatgcccatgctatggaaactttaacggtgaaaaccggcttacaacaaa

SEQ ID NO: 364 - DNA, 153 bp, synthetic construct
  source 1..153: mol_type = other DNA
  misc_difference 1..20: note = a, c, t, g, unknown or other
nnnnnnnnnnnnnnnnnnnngtcattaacccgaaagggttaatgagcagagcagtgacatgtcattcgccgcggggtgattccaagctccgcgctccggctagacatgcccatgctatggaaactttaacggtgaaaaccggcttacaacaaa

SEQ ID NO: 365 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttattgagcagagcagtgacatgtcattcgccgcggggtgattccaagctccgcgctccggctagacatgcccatgctatggaaactttaacggtgaaaaccggcttacaacaaa

SEQ ID NO: 366 - DNA, 153 bp, synthetic construct
  source 1..153: mol_type = other DNA
  misc_difference 1..20: note = a, c, t, g, unknown or other
nnnnnnnnnnnnnnnnnnnnggcaataacccgaaagggttactgcgcagagcagtgacatgtcattcgccgcggggtgattccaagctccgcgctccggctagacatgcccatgctatggaaactttaacggtgaaaaccggcttacaacaaa

SEQ ID NO: 367 - DNA, 153 bp, synthetic construct
  source 1..153: mol_type = other DNA
  misc_difference 1..20: note = a, c, t, g, unknown or other
nnnnnnnnnnnnnnnnnnnngccaataacccgaaagggttactgggcagagcagtgacatgtcattcgccgcggggtgattccaagctccgcgctccggctagacatgcccatgctatggaaactttaacggtgaaaaccggcttacaacaaa

SEQ ID NO: 368 - DNA, 153 bp, synthetic construct
  source 1..153: mol_type = other DNA
  misc_difference 1..20: note = a, c, t, g, unknown or other
nnnnnnnnnnnnnnnnnnnngtcgataacccgaaagggttaccgagcagagcagtgacatgtcattcgccgcggggtgattccaagctccgcgctccggctagacatgcccatgctatggaaactttaacggtgaaaaccggcttacaacaaa

SEQ ID NO: 369 - DNA, 153 bp, synthetic construct
  source 1..153: mol_type = other DNA
  misc_difference 1..20: note = a, c, t, g, unknown or other
nnnnnnnnnnnnnnnnnnnngtccataacccgaaagggttacggagcagagcagtgacatgtcattcgccgcggggtgattccaagctccgcgctccggctagacatgcccatgctatggaaactttaacggtgaaaaccggcttacaacaaa

SEQ ID NO: 370 - DNA, 153 bp, synthetic construct
  source 1..153: mol_type = other DNA
  misc_difference 1..20: note = a, c, t, g, unknown or other
nnnnnnnnnnnnnnnnnnnngtcaagaacccgaaagggttcctgagcagagcagtgacatgtcattcgccgcggggtgattccaagctccgcgctccggctagacatgcccatgctatggaaactttaacggtgaaaaccggcttacaacaaa

SEQ ID NO: 371 - DNA, 153 bp, synthetic construct
  source 1..153: mol_type = other DNA
  misc_difference 1..20: note = a, c, t, g, unknown or other
nnnnnnnnnnnnnnnnnnnngtcaacaacccgaaagggttgctgagcagagcagtgacatgtcattcgccgcggggtgattccaagctccgcgctccggctagacatgcccatgctatggaaactttaacggtgaaaaccggcttacaacaaa

SEQ ID NO: 372 - DNA, 153 bp, synthetic construct
  source 1..153: mol_type = other DNA
  misc_difference 1..20: note = a, c, t, g, unknown or other
nnnnnnnnnnnnnnnnnnnngtcaatgacccgaaagggtcactgagcagagcagtgacatgtcattcgccgcggggtgattccaagctccgcgctccggctagacatgcccatgctatggaaactttaacggtgaaaaccggcttacaacaaa

SEQ ID NO: 373 - DNA, 153 bp, synthetic construct
  source 1..153: mol_type = other DNA
  misc_difference 1..20: note = a, c, t, g, unknown or other
nnnnnnnnnnnnnnnnnnnngtcaatcacccgaaagggtgactgagcagagcagtgacatgtcattcgccgcggggtgattccaagctccgcgctccggctagacatgcccatgctatggaaactttaacggtgaaaaccggcttacaacaaa

SEQ ID NO: 374 - DNA, 153 bp, synthetic construct
  source 1..153: mol_type = other DNA
  misc_difference 1..20: note = a, c, t, g, unknown or other
nnnnnnnnnnnnnnnnnnnngtcaatagcccgaaagggctactgagcagagcagtgacatgtcattcgccgcggggtgattccaagctccgcgctccggctagacatgcccatgctatggaaactttaacggtgaaaaccggcttacaacaaa

SEQ ID NO: 375 - DNA, 153 bp, synthetic construct
  source 1..153: mol_type = other DNA
  misc_difference 1..20: note = a, c, t, g, unknown or other
nnnnnnnnnnnnnnnnnnnngtcaataccccgaaaggggtactgagcagagcagtgacatgtcattcgccgcggggtgattccaagctccgcgctccggctagacatgcccatgctatggaaactttaacggtgaaaaccggcttacaacaaa

SEQ ID NO: 376 - DNA, 151 bp, synthetic construct
  source 1..151: mol_type = other DNA
  misc_difference 1..20: note = a, c, t, g, unknown or other
nnnnnnnnnnnnnnnnnnnngtcaatacccgaaagggtactgagcagagcagtgacatgtcattcgccgcggggtgattccaagctccgcgctccggctagacatgcccatgctatggaaactttaacggtgaaaaccggcttacaacaaa

SEQ ID NO: 377 - DNA, 149 bp, synthetic construct
  source 1..149: mol_type = other DNA
  misc_difference 1..20: note = a, c, t, g, unknown or other
nnnnnnnnnnnnnnnnnnnngtcaatcccgaaagggactgagcagagcagtgacatgtcattcgccgcggggtgattccaagctccgcgctccggctagacatgcccatgctatggaaactttaacggtgaaaaccggcttacaacaaa

SEQ ID NO: 378 - DNA, 147 bp, synthetic construct
  source 1..147: mol_type = other DNA
  misc_difference 1..20: note = a, c, t, g, unknown or other
nnnnnnnnnnnnnnnnnnnngtcaacccgaaagggctgagcagagcagtgacatgtcattcgccgcggggtgattccaagctccgcgctccggctagacatgcccatgctatggaaactttaacggtgaaaaccggcttacaacaaa

SEQ ID NO: 379 - DNA, 152 bp, synthetic construct
  source 1..152: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccggcttacaacaa

SEQ ID NO: 380 - DNA, 151 bp, synthetic construct
  source 1..151: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccggcttacaaca

SEQ ID NO: 381 - DNA, 150 bp, synthetic construct
  source 1..150: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccggcttacaac

SEQ ID NO: 382 - DNA, 149 bp, synthetic construct
  source 1..149: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccggcttacaa

SEQ ID NO: 383 - DNA, 148 bp, synthetic construct
  source 1..148: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccggcttaca

SEQ ID NO: 384 - DNA, 145 bp, synthetic construct
  source 1..145: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccggctt

SEQ ID NO: 385 - DNA, 144 bp, synthetic construct
  source 1..144: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccggct

SEQ ID NO: 386 - DNA, 143 bp, synthetic construct
  source 1..143: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccggc

SEQ ID NO: 387 - DNA, 142 bp, synthetic construct
  source 1..142: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccgg

SEQ ID NO: 388 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagccccgcgctccggctagacatgcccatgctatggaaactttaacggtgaaaaccggcttacaacaaa

SEQ ID NO: 389 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggcgtgattccaagcgccgcgctccggctagacatgcccatgctatggaaactttaacggtgaaaaccggcttacaacaaa

SEQ ID NO: 390 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggagtgattccaagctccgcgctccggctagacatgcccatgctatggaaactttaacggtgaaaaccggcttacaacaaa

SEQ ID NO: 391 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggcgattccaagctccgcgctccggctagacatgcccatgctatggaaactttaacggtgaaaaccggcttacaacaaa

SEQ ID NO: 392 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggggattccaacctccgcgctccggctagacatgcccatgctatggaaactttaacggtgaaaaccggcttacaacaaa

SEQ ID NO: 393 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaaactccgcgctccggctagacatgcccatgctatggaaactttaacggtgaaaaccggcttacaacaaa

SEQ ID NO: 394 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcatccgccgcggggtgattccaagctccgcgctccggctagacatgcccatgctatggaaactttaacggtgaaaaccggcttacaacaaa

SEQ ID NO: 395 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcatgcgccgcggggtgattccaagctccgcgctccgcctagacatgcccatgctatggaaactttaacggtgaaaaccggcttacaacaaa

SEQ ID NO: 396 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccaagctccgcgctccgactagacatgcccatgctatggaaactttaacggtgaaaaccggcttacaacaaa

SEQ ID NO: 397 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgaatccaagctccgcgctccggctagacatgcccatgctatggaaactttaacggtgaaaaccggcttacaacaaa

SEQ ID NO: 398 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgactccaagctccgcgctccggctagacatgcccatgctatggagactttaacggtgaaaaccggcttacaacaaa

SEQ ID NO: 399 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgagtccaagctccgcgctccggctagacatgcccatgctatggacactttaacggtgaaaaccggcttacaacaaa

SEQ ID NO: 400 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgataccaagctccgcgctccggctagacatgcccatgctatggaaactttaacggtgaaaaccggcttacaacaaa

SEQ ID NO: 401 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgatcccaagctccgcgctccggctagacatgcccatgctatgggaactttaacggtgaaaaccggcttacaacaaa

SEQ ID NO: 402 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgatgccaagctccgcgctccggctagacatgcccatgctatggcaactttaacggtgaaaaccggcttacaacaaa

SEQ ID NO: 403 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgaccccaagctccgcgctccggctagacatgcccatgctatggggactttaacggtgaaaaccggcttacaacaaa

SEQ ID NO: 404 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgagcccaagctccgcgctccggctagacatgcccatgctatgggcactttaacggtgaaaaccggcttacaacaaa

SEQ ID NO: 405 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgacgccaagctccgcgctccggctagacatgcccatgctatggcgactttaacggtgaaaaccggcttacaacaaa

SEQ ID NO: 406 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgaggccaagctccgcgctccggctagacatgcccatgctatggccactttaacggtgaaaaccggcttacaacaaa

SEQ ID NO: 407 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattcctagctccgcgctccggctagacatgcccatgctatggaaactttaacggtgaaaaccggcttacaacaaa

SEQ ID NO: 408 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattcccagctccgcgctccggctagacatgcccatgctagggaaactttaacggtgaaaaccggcttacaacaaa

SEQ ID NO: 409 - DNA, 153 bp, synthetic construct
  source 1..153: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgattccgagctccgcgctccggctagacatgcccatgctacggaaactttaacggtgaaaaccggcttacaacaaa

SEQ ID NO: 410 - DNA, 154 bp, synthetic construct
  source 1..154: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgatttccaagctccgcgctccggctagacatgcccatgctatggaaactttaacggtgaaaaccggcttacaacaaa

SEQ ID NO: 411 - DNA, 155 bp, synthetic construct
  source 1..155: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgactttccaagctccgcgctccggctagacatgcccatgctatggaaactttaacggtgaaaaccggcttacaacaaa

SEQ ID NO: 412 - DNA, 155 bp, synthetic construct
  source 1..155: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgaatttccaagctccgcgctccggctagacatgcccatgctatggaaactttaacggtgaaaaccggcttacaacaaa

SEQ ID NO: 413 - DNA, 156 bp, synthetic construct
  source 1..156: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgacctttccaagctccgcgctccggctagacatgcccatgctatggaaactttaacggtgaaaaccggcttacaacaaa

SEQ ID NO: 414 - DNA, 156 bp, synthetic construct
  source 1..156: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgatatttccaagctccgcgctccggctagacatgcccatgctatggaaactttaacggtgaaaaccggcttacaacaaa

SEQ ID NO: 415 - DNA, 155 bp, synthetic construct
  source 1..155: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgagtttccaagctccgcgctccggctagacatgcccatgctatggaaactttaacggtgaaaaccggcttacaacaaa

SEQ ID NO: 416 - DNA, 156 bp, synthetic construct
  source 1..156: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgaagtttccaagctccgcgctccggctagacatgcccatgctatggaaactttaacggtgaaaaccggcttacaacaaa

SEQ ID NO: 417 - DNA, 156 bp, synthetic construct
  source 1..156: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgacgtttccaagctccgcgctccggctagacatgcccatgctatggaaactttaacggtgaaaaccggcttacaacaaa

SEQ ID NO: 418 - DNA, 156 bp, synthetic construct
  source 1..156: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgatgtttccaagctccgcgctccggctagacatgcccatgctatggaaactttaacggtgaaaaccggcttacaacaaa

SEQ ID NO: 419 - DNA, 156 bp, synthetic construct
  source 1..156: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgaggtttccaagctccgcgctccggctagacatgcccatgctatggaaactttaacggtgaaaaccggcttacaacaaa

SEQ ID NO: 420 - DNA, 157 bp, synthetic construct
  source 1..157: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgaaagtttccaagctccgcgctccggctagacatgcccatgctatggaaactttaacggtgaaaaccggcttacaacaaa

SEQ ID NO: 421 - DNA, 157 bp, synthetic construct
  source 1..157: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgagagtttccaagctccgcgctccggctagacatgcccatgctatggaaactttaacggtgaaaaccggcttacaacaaa

SEQ ID NO: 422 - DNA, 157 bp, synthetic construct
  source 1..157: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgaaggtttccaagctccgcgctccggctagacatgcccatgctatggaaactttaacggtgaaaaccggcttacaacaaa

SEQ ID NO: 423 - DNA, 157 bp, synthetic construct
  source 1..157: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgactgtttccaagctccgcgctccggctagacatgcccatgctatggaaactttaacggtgaaaaccggcttacaacaaa

SEQ ID NO: 424 - DNA, 157 bp, synthetic construct
  source 1..157: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgatcgtttccaagctccgcgctccggctagacatgcccatgctatggaaactttaacggtgaaaaccggcttacaacaaa

SEQ ID NO: 425 - DNA, 158 bp, synthetic construct
  source 1..158: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgaaaagtttccaagctccgcgctccggctagacatgcccatgctatggaaactttaacggtgaaaaccggcttacaacaaa

SEQ ID NO: 426 - DNA, 159 bp, synthetic construct
  source 1..159: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgagaaagtttccaagctccgcgctccggctagacatgcccatgctatggaaactttaacggtgaaaaccggcttacaacaaa

SEQ ID NO: 427 - DNA, 161 bp, synthetic construct
  source 1..161: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgatagaaagtttccaagctccgcgctccggctagacatgcccatgctatggaaactttaacggtgaaaaccggcttacaacaaa

SEQ ID NO: 428 - DNA, 161 bp, synthetic construct
  source 1..161: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgaatgaaagtttccaagctccgcgctccggctagacatgcccatgctatggaaactttaacggtgaaaaccggcttacaacaaa

SEQ ID NO: 429 - DNA, 161 bp, synthetic construct
  source 1..161: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgaacgaaagtttccaagctccgcgctccggctagacatgcccatgctatggaaactttaacggtgaaaaccggcttacaacaaa

SEQ ID NO: 430 - DNA, 161 bp, synthetic construct
  source 1..161: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgacagaaagtttccaagctccgcgctccggctagacatgcccatgctatggaaactttaacggtgaaaaccggcttacaacaaa

SEQ ID NO: 431 - DNA, 163 bp, synthetic construct
  source 1..163: mol_type = other DNA
  misc_difference 1..20: note = a, c, t, g, unknown or other
nnnnnnnnnnnnnnnnnnnngtcaataacccgaaagggttactgagcagagcagtgacatgtcattcgccgcggggtgatgtgaaaagtttccaagctccgcgctccggctagacatgcccatgctatggaaactttaacggtgaaaaccggcttacaacaaa

SEQ ID NO: 432 - protein, 5 aa, synthetic construct
FTGKS

SEQ ID NO: 433 - protein, 4 aa, synthetic construct
KHND

SEQ ID NO: 434 - RNA, 225 nt, Homo sapiens
  source 1..225: mol_type = genomic RNA
  misc_difference 1..20: note = a, c, u, g, unknown or other
nnnnnnnnnnnnnnnnnnnnggctcttccaactttatggttgcgaccgtaggttgaaagagcacaggctgagacattcgtaaggccgaaagaccggacgcaccctgggataccccttgtccccggaactgcatagcggatgccagttgatggagcaatctatcagataagccagggggaacaatcacctctctgtatcagagagattttacaaaaggaggaacgg

SEQ ID NO: 435 - RNA, 64 nt, Homo sapiens
  source 1..64: mol_type = genomic RNA
caataacccatgactgaagtcatgggcttgcagatgcaggtcctgatggaagaaagggttactg

SEQ ID NO: 436 - RNA, 161 nt, Homo sapiens
  source 1..161: mol_type = genomic RNA
  misc_difference 1..20: note = a, c, u, g, unknown or other
nnnnnnnnnnnnnnnnnnnngtagcagagcagtgacatgtcattcgccgcggggtgattccaagctccgcgctccggctagacatgcccatgctatggaaactttaacggtatgtgcggttttccgctcataccggcttacaacaaataaggagttattag

SEQ ID NO: 437 - RNA, 153 nt, synthetic construct
  source 1..153: mol_type = other RNA
  misc_difference 1..20: note = a, c, u, g, unknown or other
nnnnnnnnnnnnnnnnnnnngtcaataacccgaaagggttactgagcagagcagtgacatgtcattcgccgcggggtgattccaagctccgcgctccggctagacatgcccatgctatggaaactttaacggtgaaaaccggcttacaacaaa

SEQ ID NO: 438 - protein, 224 aa, Petromyzon marinus; identical to SEQ ID NO: 129

SEQ ID NO: 439 - RNA, 10 nt, synthetic construct
  source 1..10: mol_type = other RNA
  modified_base 1: mod_base = OTHER; note = thymine
  modified_base 3: mod_base = OTHER; note = thymine
tgtgaaaagt

(19) United States
(12) Patent Application Publication
(10) Pub. No.: US 2026/0286324 A1
(43) Pub. Date: Sep. 24, 2026

(54) MODIFIED ISCB NUCLEASES, MODIFIED GUIDE RNAS, AND USES THEREOF

(71) Applicant: AERA THERAPEUTICS, INC., Cambridge, MA (US)

(72) Inventors: Liyang ZHANG, Cambridge, MA (US); Max Atticus ENGLISH, Cambridge, MA (US); Colin McGAW, Cambridge, MA (US); Hannah KEMPTON, Cambridge, MA (US); Sean BENLER, Cambridge, MA (US); Ian SLAYMAKER, Cambridge, MA (US)

(73) Assignee: AERA THERAPEUTICS, INC., Cambridge, MA (US)

(21) Appl. No.: 19/471,169

(22) PCT Filed: Mar. 29, 2024
(86) PCT No.: PCT/US2024/022237

§ 371 Date: Sep. 30, 2025

Related U.S. Application Data
(60) Provisional application No. 63/456,332, filed on Mar. 31, 2023.
(60) Provisional application No. 63/456,322, filed on Mar. 31, 2023.

(51) Int. Cl. C12N 9/22; A61K 38/00; C12N 15/11; C12N 15/88; C12N 15/90

(52) U.S. Cl. CPC C12N 9/22; C12N 15/11; C12N 15/88; C12N 15/907; A61K 38/00; C12N 2310/20; C12N 2310/311; C12N 2310/313; C12N 2310/314; C12N 2310/315; C12N 2310/318; C12N 2310/321; C12N 2310/322; C12N 2310/3231; C12N 2310/332; C12N 2310/334; C12N 2310/3341; C12N 2310/335; C12N 2310/336; C12N 2310/531

(57) ABSTRACT

Disclosed herein are novel polypeptides having nuclease activity. The polypeptides can effect modifications at defined locations within target nucleic acids, e.g., DNA and RNA. Also disclosed herein modified guide RNAs. The polypeptides and guide RNAs are useful in compositions, systems and methods, to make site specific alterations of target nucleic acid sequences.

### CROSS-REFERENCE TO RELATED APPLICATIONS

This application is a 35 U.S.C. § 371 National Phase Entry Application of International Patent Application No. PCT/US2024/022237 filed on Mar. 29, 2024, which designated the U.S., and which claims benefit under 35 U.S.C. § 119 (e) of U.S. Provisional Application No. 63/456,332 filed Mar. 31, 2023, and U.S. Provisional Application No. 63/456,322 filed Mar. 31, 2023, the contents of each of which are incorporated herein by reference in their entireties.

### SEQUENCE LISTING

The instant application contains a Sequence Listing which has been submitted in XML format via Patent Center and is hereby incorporated by reference in its entirety. Said XML copy, created on Aug. 21, 2024, is named 098791-000102WOPT_SL.xml and is 489,447 bytes in size.

### TECHNICAL FIELD

The disclosure relates generally to DNA and RNA targeting enzymes, systems, and compositions that are useful for modifying target nucleic acid sequences.

### BACKGROUND

EboIscB (insertion sequences Cas9-like OrfB) is an RNA-guided endonuclease with measurable activity in human cells. However, the EboIscB in human cells is low. Further, to improve the utility of EboIscB as gene editing tool in human cells, guide RNAs that are shorter and have higher efficacy are needed. Thus, there remains a need in the art for RNA-guided endonucleases and guide RNAs having improved and enhanced activity, e.g., in human cells. This disclosure answers these needs.

### SUMMARY

Described herein are DNA and RNA targeting enzymes and systems, that are useful for modifying target nucleic acid sequences. Accordingly, provided herein is an engineered or non-naturally occurring DNA and RNA targeting enzymes and systems that include an engineered guide nucleic acid comprising a guide sequence (i.e., a spacer), where the guide sequence is capable of hybridizing with a target sequence of a target nucleic acid molecule.

Accordingly in one aspect provided herein is an IscB protein (also referred to as a protein effector polypeptide herein) comprising an amino acid sequence that is at least 80% identical to the amino SEQ ID NO: 2 (pEDIT071) or SEQ ID NO: 8 (pEdit071+NLS). For example, the IscB protein comprises an amino acid sequence that is at least 85%, at least 90%, at least 92%, at least 95%, at least 96%, at least 97%, at least 98%, at least 99%, or at least 99.5% identical to SEQ ID NO: 2. In some embodiments, the IscB protein comprises an amino acid sequence of SEQ ID NO: 2, and wherein the amino acid sequence of the IscB protein comprises a mutation at least one, at least two, at least three, at least four, at least five, at least six, at least seven, at least eight, or at least nine positions selected independently from 18, 34, 36, 37, 60, 66, 85, 97, 99, 100, 101, 104, 109, 112, 118, 136, 137, 155, 156, 157, 166, 168, 190, 204, 204, 205, 206, 208, 212, 214, 216, 218, 219, 229, 229, 230, 233, 233, 234, 234, 238, 239, 241, 242, 245, 246, 248, 252, 254, 255, 256, 257, 258, 279, 280, 291, 293, 298, 303, 304, 306, 307, 312, 319, 323, 328, 329, 331, 332, 338, 341, 353, 360, 368, 372, 376, 377, 378, 379, 380, 381, 382, 384, 385, 386, 388, 390, 391, 393, 405, 406, 408, 409, 410, 412, 413, 416, 417, 418, 419, 421, 421, 423, 425, 437, 439, 455, 462, 463, 478, 479, 533, 536, 564 and 565 of the amino acid sequence of SEQ ID NO: 2 (pEDIT071). Optionally, if the IscB comprises a single mutation, the mutation is not Q60I, N66H, I85R, I85K, E137R, P405R, E409R, D413K, or combination thereof.

In some embodiments, the IscB protein comprises an amino acid sequence of SEQ ID NO: 8, and wherein the amino acid sequence of the IscB protein comprises a mutation at least one, at least two, at least three, at least four, at least five, at least six, at least seven, at least eight, or at least nine positions selected independently from 30, 46, 48, 49, 72, 78, 97, 109, 111, 112, 113, 116, 121, 124, 130, 148, 149, 167, 168, 169, 178, 180, 202, 216, 216, 217, 218, 220, 224, 226, 228, 230, 231, 241, 241, 242, 245, 245, 246, 246, 250, 251, 253, 254, 257, 258, 260, 264, 266, 267, 268, 269, 270, 291, 292, 303, 305, 310, 315, 316, 318, 319, 324, 331, 335, 340, 341, 343, 344, 350, 353, 365, 372, 380, 384, 388, 389, 390, 391, 392, 393, 394, 396, 397, 398, 400, 402, 403, 405, 417, 418, 420, 421, 422, 424, 425, 428, 429, 430, 431, 433, 433, 435, 437, 449, 451, 467, 474, 475, 490, 491, 545, 548, 576, and 577 of the amino acid sequence of SEQ ID NO: 8 (pEDIT071+NLS). Optionally, if the IscB comprises a single mutation, the mutation is not Q72I, H78H, I97R, I97K, E149R, P417R, E421R, D425K, or combination thereof.

In some embodiments, the amino acid sequence of the IscB protein comprises at least one, at least two, at least three, at least four, at least five, at least six, at least seven, at least eight, or at least nine mutations selected independently from the group consisting of variant C18R, A34R, D36R, T37R, Q60I, N66H, I85X, I85X, M99X, A101X, A104R, E109X, A112K, R118K, S136X, E137R, I155X, L156X, T157X, T166X, L168K, L190K, G204X, N205X, I206K, K208R, K212R, K214R, N216R, K218R, D219X, A229X, K230R, L233X, P234X, P238X, F239X, K241R, E242X, A245X, E246X, E248R, T252X, E254X, K255R, S256X, T257X, F258X, S279R, G280R, L291X, K293R, Q298K, E303X, M304K, K306R, R307K, C312X, T319K, E323X, E328R, Q329R, D331R, G332R, G338R, S341R, R353K, A360X, K368R, L372R, D376R, A377R, D378R, T379R, A380R, E381R, S382R, T384R, E385R, M386R, T388R, L390X, M391X, K393R, P405K, K406R, V408R, E409X, T410R, A412R, D413R, P416R, G417R, H418K, F419R, V421X, N423R, W425K, E437R, D439X, E455R, E462R, P463R, I533K, T536R, H478X, H479X, I564X, and T565X of the amino acid sequence SEQ ID NO: 2, wherein X is any amino acid. Optionally, if the IscB comprises a single mutation, the mutation is not Q60I, N66H, I85R, I85K, E137R, P405R, E409R, D413K, or combination thereof.

In some embodiments, the amino acid sequence of the IscB protein comprises at least one, at least two, at least three, at least four, at least five, at least six, at least seven, at least eight, or at least nine mutations selected independently from the group consisting of variant C30R, A46R, D48R, T49R, Q72I, N78H, I97X, I109X, M111X, A1113X, A116R, E121X, A124K, R130K, S148X, E149R, I167X, L168X, T169X, T178X, L180K, L202K, G216X, N217X, I218K, K2220R, K224R, K2226R, N228R, K230R, D231X, A241X, K242R, L245X, P246X, P250X, F251X, K253R, E254X, A257X, E258X, E260R, T264X, E266X, K267R, S268X, T269X, F270X, S291R, G292R, L303X, K2305R, Q310K, E315X, M316K, K318R, R319K, C324X, T331K, E335X, E340R, Q343R, G344R, G350R, S353R, R365K, A372X, K380R, L384R, D388R, A389R, D390R, T391R, A392R, E393R, S394R, T396R, E397R, M398R, T400R, L402X, M403X, K3405R, P417K, K418R, V420R, E421X, T422R, A424R, D425R, P428R, G429R, H430K, F431R, V433X, N435R, W437K, E449R, D451X, E467R, E474R, P475R, I545K, T548R, H490X, H491X, I576X, and T577X of the amino acid sequence SEQ ID NO: 8, wherein X is any amino acid. Optionally, if the IscB comprises a single mutation, the mutation is not Q72I, H78H, I97R, I97K, E149R, P417R, E421R, D425K, or combination thereof.

In some embodiments, the amino acid sequence of the IscB protein comprises at least one, at least two, at least three, at least four, at least five, at least six, at least seven, at least eight, or at least nine mutations selected independently from the group consisting of C18R, A34R, D36R, T37R, Q60I, N66H, I85R, I85K, D97R, D97K, M99R, M99K, A100K, A101R, A101K, A104R, E109R, E109K, A112K, R118K, S136R, S136K, E137R, E137K, I155R, 1155K, L156R, L156K, T157R, T157K, T166R, T166K, L168K, L190K, G204R, G204K, N205R, N205K, I206K, K208R, K212R, K214R, N216R, K218R, D219R, D219K, A229R, A229K, K230R, L233R, L233K, P234R, P234K, P238R, P238K, F239R, F239K, K241R, E242R, E242K, A245R, A245K, E246R, E246K, E248R, T252R, T252K, E254R, E254K, K255R, S256R, S256K, T257R, T257K, F258R, F258K, S279R, G280R, L291R, L291K, K293R, Q298K, E303R, E303K, M304K, K306R, R307K, C312R, C312K, T319K, E323R, E323K, E328R, Q329R, D331R, G332R, G338R, S341R, R353K, A360R, A360K, K368R, L372R, D376R, A377R, D378R, T379R, A380R, E381R, S382R, T384R, E385R, M386R, T388R, L390R, L390K, M391R, M391K, K393R, P405K, K406R, V408R, E409K, E409R, T410R, A412R, D413R, D413K, P416R, G417R, H418K, F419R, V421R, V421K, N423R, W425K, E437R, D439R, D439K, E455R, E462R, P463R, I533K, T536R, H478N, H479N, I564K, I564H, I564N, I564Q, I564C, I564M, I564T, I564D, T565R, T565K, T565H, T565C, T565D, T565G, H478C, H478P, H479C, and H479M of the amino acid sequence provided in SEQ ID NO: 2. Optionally, if the IscB comprises a single mutation, the mutation is not Q60I, N66H, I85R, I85K, E137R, P405R, E409R, D413K, or combination thereof.

In some embodiments, the amino acid sequence of the IscB protein comprises at least one, at least two, at least three, at least four, at least five, at least six, at least seven, at least eight, or at least nine mutations selected independently from the group consisting of C30R, A46R, D46R, T49R, Q72I, N78H, I97R, I97K, D109R, D109K, M111R, M111K, A112K, A113R, A113K, A116R, E121R, E121K, A124K, R130K, S148R, S148K, E149R, E149K, 1167R, 1167K, L168R, L168K, T169R, T169K, T178R, T178K, L180K, L1202K, G216R, G216K, N217R, N217K, I218K, K220R, K224R, K2226R, N228R, K230R, D231R, D231K, A241R, A241K, K242R, L245R, L245K, P246R, P246K, P250R, P250K, F251R, F251K, K253R, E254R, E2454K, A257R, A257K, E258R, E2458K, E260R, T264R, T264K, E266R, E266K, K267R, S268R, S268K, T2569R, T2569K, F270R, F270K, S291R, G292R, L303R, L303K, K305R, Q310K, E315R, E315K, M316K, K318R, R319K, C324R, C324K, T331K, E335R, E335K, E340R, Q341R, D343R, G344R, G350R, S353R, R365K, A372R, A372K, K380R, L384R, D388R, A389R, D390R, T391R, A392R, E393R, S394R, T396R, E397R, M398R, T400R, L402R, L402K, M4031R, M403K, K405R, P417K, K419R, V420R, E421K, E421R, T422R, A424R, D425R, D425K, P428R, G429R, H430K, F431R, V433R, V433K, N435R, W437K, E449R, D451R, D451K, E4567R, E474R, P475R, I545K, T548R, H490N, H491N, I576K, I576H, I576N, 1576Q, I576C, I576M, I576T, I5676D, T577R, T577K, T577H, T577C, T577D, T577G, H490C, H490P, H491C, and H491M of the amino acid sequence provided in SEQ ID NO: 8. Optionally, if the IscB comprises a single mutation, the mutation is not Q72I, H78H, I97R, I97K, E149R, P417R, E421R, D425K, or combination thereof.

In some embodiments, the IscB comprises an amino acid sequence comprising at least one, at least two, at least 3 or at least 4 mutations selected from the group consisting of H478X, H479N, I564X, T565X, H479C, and H479M of the amino acid sequence provided in SEQ ID NO: 2, wherein X is any amino acid. In some embodiments, the IscB comprises an amino acid sequence comprising at least one, at least two, at least 3 or at least 4 mutations selected from the group consisting of H478N, H479N, I564K, I564H, I564N, I564Q, I564C, I564M, I564T, I564D, T565R, T565K, T565H, T565C, T565D, T565G, H478C, H478P, H479C, and H479M of the amino acid sequence provided in SEQ ID NO: 2.

In some embodiments, the IscB protein comprises an amino acid sequence comprising a mutation at position 478 and/or 479 of the amino acid sequence provided in SEQ ID NO: 2. In some embodiments, the IscB protein comprises an amino acid sequence comprising the mutation H478N, H479N, H478C and/or H478P in the amino acid sequence provided in SEQ ID NO: 2.

In some embodiments, the IscB protein comprises an amino acid sequence comprising the mutations E409R, D413K, 533K in SEQ ID NO: 2. In some embodiments, the IscB protein comprises an amino acid sequence comprising the mutations E409R, D413K, and 533K, and at least one other mutation at a position listed in Table A in SEQ ID NO: 2. In some further embodiments, the IscB protein comprises an amino acid sequence comprising the mutations E409R, D413K and 533K, and at least one, at least two, at least three, or four of the mutation E137K, N66H, A100K, and/or T536R in SEQ ID NO: 2. For example, the IscB protein comprises an amino acid sequence comprising the mutations E409R, D413K, 533K, E137K, N66H, A100K, and T536R in SEQ ID NO: 2.

In some embodiments, the IscB protein comprises an amino acid sequence comprising the mutations E409R, D413K, and 533K; at least one, at least two, at least three, or four of the mutation E137K, N66H, A100K, and/or T536R; and at least one other mutation listed in Table A in SEQ ID NO: 2

In some embodiments, the IscB protein comprises an amino acid sequence comprising the mutations E409R, D413K, 533K E137K, N66H, A100K and T536R, and at least one other mutation listed in Table A in SEQ ID NO: 2.

In some embodiments, the IscB protein comprises an amino acid sequence comprising at least one, at least two, at least three, at least four, at least five, at least six or seven of the mutations E409R, D413K, 533K, E137K, N66H, A100K, and/or T536R, and at least one other mutation listed in Table A in SEQ ID NO: 2. For example, the IscB protein comprises an amino acid sequence comprising at least one, at least two, at least three, at least four, at least five, at least six or seven of the mutations E409R, D413K, 533K, E137K, N66H, A100K, and/or T536R, and at least one other mutation listed in Table A in SEQ ID NO: 2, and where the at least one other mutation is not E409R, D413K, 533K, E137K, N66H, A100K, and/or T536R.

In some embodiments, the IscB protein one, two, three or four of the mutations E137K, N66H, A100K, and/or T536R in SEQ ID NO: 2. For example, the IscB protein one, two, three or four of the mutations E137K, N66H, A100K, and/or T536R, and at least one other mutation listed in Table A in SEQ ID NO: 2.

In some embodiments, the IscB comprises an amino acid sequence comprising at least one, at least two, at least 3 or at least 4 mutations selected from the group consisting of H490X, H491N, I576X, T577X, H491C, and H491M of the amino acid sequence provided in SEQ ID NO: 7, wherein X is any amino acid. In some embodiments, the IscB comprises an amino acid sequence comprising at least one, at least two, at least 3 or at least 4 mutations selected from the group consisting of H490N, H491N, I576K, I576H, I576N, I576Q, I576C, I576M, I576T, I576D, T577R, T577K, T577H, T577C, T577D, T577G, H490C, H490P, H491C, and H491M of the amino acid sequence provided in SEQ ID NO: 7.

In some embodiments, the IscB protein comprises an amino acid sequence comprising a mutation at position 490 and/or 491 of the amino acid sequence provided in SEQ ID NO: 7. In some embodiments, the IscB protein comprises an amino acid sequence comprising the mutation H490N, H491N, H490C and/or H490P in the amino acid sequence provided in SEQ ID NO: 7.

In some embodiments, the IscB protein comprises an amino acid sequence comprising the mutations E421R, D425K, 545K in SEQ ID NO: 7. In some embodiments, the IscB protein comprises an amino acid sequence comprising the mutations E421R, D425K, and 545K, and at least one other mutation at a position listed in Table A2 in SEQ ID NO: 7. In some further embodiments, the IscB protein comprises an amino acid sequence comprising the mutations E421R, D425K and 545K, and at least one, at least two, at least three, or four of the mutation E149K, N78H, A112K, and/or T548R in SEQ ID NO: 7. For example, the IscB protein comprises an amino acid sequence comprising the mutations E421R, D425K, 545K, E149K, N78H, A112K, and T548R in SEQ ID NO: 7.

In some embodiments, the IscB protein comprises an amino acid sequence comprising the mutations E421R, D425K, and 545K; at least one, at least two, at least three, or four of the mutation E149K, N78H, A112K, and/or T548R; and at least one other mutation listed in Table A2 in SEQ ID NO: 7

In some embodiments, the IscB protein comprises an amino acid sequence comprising the mutations E421R, D425K, 545K E149K, N78H, A112K and T548R, and at least one other mutation listed in Table A2 in SEQ ID NO: 7.

In some embodiments, the IscB protein comprises an amino acid sequence comprising at least one, at least two, at least three, at least four, at least five, at least six or seven of the mutations E421R, D425K, 545K, E149K, N78H, A112K, and/or T548R, and at least one other mutation listed in Table A2 in SEQ ID NO: 7. For example, the IscB protein comprises an amino acid sequence comprising at least one, at least two, at least three, at least four, at least five, at least six or seven of the mutations E421R, D425K, 545K, E149K, N78H, A112K, and/or T548R, and at least one other mutation listed in Table A2 in SEQ ID NO: 7, and where the at least one other mutation is not E421R, D425K, 545K, E149K, N78H, A112K, and/or T548R.

In some embodiments, the IscB protein one, two, three or four of the mutations E149K, N78H, A112K, and/or T548R in SEQ ID NO: 7. For example, the IscB protein one, two, three or four of the mutations E149K, N78H, A112K, and/or T548R, and at least one other mutation listed in Table A2 in SEQ ID NO: 7.

Without wishing to be bound by a theory, in some embodiments, the IscB protein exhibits an increased activity on a target sequence that does not comprise the canonical Target Adjacent Motif (TAM) sequence 5′-ATAAA-3′ at its 3′ end as compared to IscB as provided by SEQ ID NO: 2. For example, the IscB protein exhibits an activity that is at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of IscB as provided by SEQ ID NO: 2 on a target sequence having a 3′ end that is not directly adjacent to the canonical TAM sequence (5′-ATAAA-3′). The activity can be measured by a nuclease assay, a deamination assay, or a transcriptional activation assay.

In some embodiments, the IscB protein described herein exhibits an increased activity on a target sequence comprising, at its 3′ end, the TAM sequence RTR (e.g., ATR) as compared to SEQ ID NO: 2, (where R is purine (e.g., A or G) and R is A or C. For example, the IscB protein described herein exhibits an increased activity on a target sequence comprising, at its 3′ end, a TAM sequence selected from the group consisting of RTAAA, ATRAA, ATRMM, ATRA, ATRRA, ATARA, ATRRAC, ATAAAC, ATARM, ATRAM, ATRRM, ATRMA, ATANA, ATRR, ATR, ATGAC, ATAA, ATAR, ATGMC, ATGAA, and ATRAR at its 3′ end as compared to IscB as provided by SEQ ID NO: 2, wherein R is purine (e.g., A or G) and R is A or C.

In some embodiments, the IscB comprises an amino acid sequence comprising at least one, at least two, at least 3 or at least 4 mutations selected from the group consisting of H478X, H479N, I564X, T565X, H479C, and H479M of the amino acid sequence provided in SEQ ID NO: 2, wherein X is any amino acid. In some embodiments, the IscB comprises an amino acid sequence comprising at least one, at least two, at least 3 or at least 4 mutations selected from the group consisting of H478N, H479N, I564K, I564H, I564N, I564Q, I564C, I564M, I564T, I564D, T565R, T565K, T565H, T565C, T565D, T565G, H478C, H478P, H479C, and H479M of the amino acid sequence provided in SEQ ID NO: 2.

Without wishing to be bound by a theory, the IscB described herein exhibits an increased activity on a target sequence comprising the TAM sequence ATR. For example, the IscB comprises an amino acid sequence comprising at least one, at least two, at least 3 or at least 4 mutations selected from the group consisting of H478N, H479N, I564K, I564H, I564N, I564Q, I564C, I564M, I564T, I564D, T565R, T565K, T565H, T565C, T565D, T565G, H478C, H478P, H479C, and H479M of the amino acid sequence provided in SEQ ID NO: 2, and exhibits an increased activity on a target sequence comprising the TAM sequence ATR compared to the activity of SEQ ID NO: 2.

In some embodiments, the IscB comprises an amino acid sequence comprising at least one, at least two, at least 3 or at least 4 mutations selected from the group consisting of H490X, H491N, I576X, T577X, H491C, and H491M of the amino acid sequence provided in SEQ ID NO: 8, wherein X is any amino acid. In some embodiments, the IscB comprises an amino acid sequence comprising at least one, at least two, at least 3 or at least 4 mutations selected from the group consisting of H490N, H491N, I576K, I576H, I576N, I576Q, I576C, I576M, I576T, I576D, T577R, T577K, T577H, T577C, T577D, T577G, H490C, H490P, H491C, and H491M of the amino acid sequence provided in SEQ ID NO: 8.

Without wishing to be bound by a theory, the IscB described herein exhibits an increased activity on a target sequence comprising the TAM sequence ATR. For example, the IscB comprises an amino acid sequence comprising at least one, at least two, at least 3 or at least 4 mutations selected from the group consisting of H490N, H491N, I576K, I576H, I576N, I576Q, I576C, I576M, I576T, I576D, T577R, T577K, T577H, T577C, T577D, T577G, H490C, H490P, H491C, and H491M of the amino acid sequence provided in SEQ ID NO: 8, and exhibits an increased activity on a target sequence comprising the TAM sequence ATR compared to the activity of SEQ ID NO: 8.

In some embodiments, the IscB protein comprises an amino acid sequence comprising at least 2, at least 3, at least 4, at least 5, at least 6, at least 7, at least 8, at least 9, at least 10, or at least amino sequence swap at position 560-571 in SEQ ID NO: 2. For example, the IscB protein comprises an amino acid sequence comprising at least 2, at least 3, at least 4, at least 5 or at least six amino sequence swap at position Q563-K568 in SEQ ID NO. 2. Without wishing to be bound by a theory, a sequence swap at positions 560-571 (e.g., at position 563-568) leads to enhanced or increased activity on a target sequence comprising the TAM sequence RTAAA. For example, the IscB protein comprises an amino acid sequence comprising the sequence QTSGGR (SEQ ID NO: 11), RSSGGK (SEQ ID NO: 12), QQSKGR (SEQ ID NO: 13), QQGRGR (SEQ ID NO: 14), QQNKGR (SEQ ID NO: 15) or QKDKGA (SEQ ID NO: 16) at position Q563-K568 in the SEQ ID NO: 2, and wherein the IscB protein exhibits an increased activity on a target sequence comprising the TAM sequence RTAAA.

In some embodiments, the IscB protein comprises an amino acid sequence comprising at least 2, at least 3, at least 4, at least 5, at least 6, at least 7, at least 8, at least 9, at least 10, or at least amino sequence swap at position 572-583 in SEQ ID NO: 8. For example, the IscB protein comprises an amino acid sequence comprising at least 2, at least 3, at least 4, at least 5 or at least six amino sequence swap at position Q575-K580 in SEQ ID NO. 8. Without wishing to be bound by a theory, a sequence swap at positions 572-583 (e.g., at position 575-580) leads to enhanced or increased activity on a target sequence comprising the TAM sequence RTAAA. For example, the IscB protein comprises an amino acid sequence comprising the sequence QTSGGR (SEQ ID NO: 11), RSSGGK (SEQ ID NO: 12), QQSKGR (SEQ ID NO: 13), QQGRGR (SEQ ID NO: 14), QQNKGR (SEQ ID NO: 15) or QKDKGA (SEQ ID NO: 16) at position Q575-K580 in the SEQ ID NO: 8, and wherein the IscB protein exhibits an increased activity on a target sequence comprising the TAM sequence RTAAA.

Without wishing to be bound by a theory, the IscB proteins described herein exhibit lower off-target activity as compared to an off-target activity of the IscB domain as provided by SEQ ID NO: 2 or 8.

In another aspect, provided herein is a fusion protein comprising an IscB protein described herein and an effector domain (also referred to as a functional domain herein). Some exemplary effector domains include, but are not limited to, domains or functional fragments thereof comprising nuclease activity, nickase activity, recombinase activity, deaminase activity, methyltransferase activity, methylase activity, acetylase activity, acetyltransferase activity, transcriptional activation activity, or transcriptional repression activity.

In some embodiments, the effector domain is a nucleic acid editing domain. For example, the effector domain is reverse transcriptase, a deaminase, or a functional fragment thereof. In some embodiments, the effector domain is a reverse transcriptase or a functional fragment thereof.

In some embodiments, the effector domain is a deaminase domain or a functional fragment thereof. In some embodiments, the deaminase domain is a cytidine deaminase domain or a functional fragment thereof. A preferred cytidine deaminase domain is an apolipoprotein B mRNA-editing complex (APOBEC) family deaminase or a functional fragment thereof. In some embodiments, the cytidine deaminase domain comprises an amino acid sequence having at least 80%, at least 85%, at least 90%, at least 92%, at least 95%, at least 96%, at least 97%, at least 98%, at least 99%, at least 99.5%, or 100% identical to any one of the sequences shown in Table D1.

In some embodiments, the deaminase domain is an adenosine deaminase domain or a functional fragment thereof. In some embodiments, the adenosine deaminase domain comprises an amino acid sequence having at least 80%, at least 85%, at least 90%, at least 92%, at least 95%, at least 96%, at least 97%, at least 98%, at least 99%, at least 99.5%, or 100% identical to any one of the sequences shown in Table D2. In some embodiments, the adenosine deaminase is an ecTadA domain, or variant thereof.

In some embodiments, the fusion protein comprises two adenosine deaminase domains. The first and second adenosine domains can comprise independently an amino acid sequence having at least 80%, at least 85%, at least 90%, at least 92%, at least 95%, at least 96%, at least 97%, at least 98%, at least 99%, at least 99.5%, or 100% identical to any one of the sequences shown in Table D2. In some embodiments, the first adenosine deaminase domain and the second adenosine deaminase domain comprises an ecTadA domain, or variant thereof.

In some embodiments, the effector domain is a uracil glycosylase inhibitor (UGI) domain or a functional fragment thereof. The UGI domain can comprise an amino acid sequence having at least 80%, at least 85%, at least 90%, at least 92%, at least 95%, at least 96%, at least 97%, at least 98%, at least 99%, at least 99.5%, or 100% identical to any one of the sequences shown in Table E.

In another aspect provided herein is complex comprising an IscB protein described herein and a guide nucleic acid (e.g., guide RNA or ωRNA). For example, the complex comprises a fusion protein described herein bound/complexed with a guide nucleic acid. Without wishing to be bound by a theory, the complex can be useful for editing a point mutation in a target sequence.

The guide nucleic acids are described herein below. Generally, the guide nucleic acid is from about 15 nucleotides to about 100 nucleotides in length. For example, the guide nucleic acid can be 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long.in length. Typically, the guide nucleic acid comprises a sequence (guide sequence) of at least 10 contiguous nucleotides that is complementary to a target sequence. For example, the guide nucleic acid comprises a sequence (guide sequence) of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

In some embodiments, the guide nucleic acid is from about 15 nucleotides to about 100 nucleotides in length and comprises a sequence (guide sequence) of at least 10 contiguous nucleotides that is complementary to a target sequence.

The target sequence can be a DNA sequence. In some embodiments, the target sequence is a sequence in the genome of a mammal. In some embodiments, the target sequence is a sequence in the genome of a human.

In some embodiments, the target sequence comprises a sequence associated with a disease or disorder. For example, the target sequence comprises a point mutation associated with a disease or disorder. The point mutation can be located between about 10 to about 20 nucleotides upstream of the TAM in the target sequence.

In some embodiments, the target sequence comprises a T to C point mutation. In some embodiments, a complex described herein deaminates the target C point mutation, and wherein the deamination results in a sequence that is not associated with a disease or disorder. In some embodiments, the target C point mutation is present in the DNA strand that is not complementary to the guide RNA. In some embodiments, the target sequence comprises a G to A point mutation. In some embodiments, the complex deaminates the target A point mutation, and wherein the deamination results in a sequence that is not associated with a disease or disorder. In some embodiments, the target A point mutation is present in the DNA strand that is not complementary to the guide RNA.

In some embodiments, the complex exhibits increased deamination efficiency of a point mutation in a target sequence that does not comprise the canonical TAM (5′-ATAAA-3′) at its 3′ end as compared to the deamination efficiency of a complex comprising IscB protein as provided by SEQ ID NO: 2. In some embodiments, the complex exhibits increased deamination efficiency of a point mutation in a target sequence having a 3′ end that is not directly adjacent to the canonical TAM sequence (5′-ATAAA-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the deamination efficiency of complex comprising the IscB protein as provided by SEQ ID NO: 2 on the same target sequence. In some embodiments, a deamination activity is measured using a deamination assay, PCR, or sequencing.

In some embodiments, the complex produces fewer indels in a target sequence that does not comprise the canonical TAM (5′-ATAAA-3′) at its 3′ end as compared to the amount of indels produced by a complex comprising IscB protein as provided by SEQ ID NO: 2 or 8. In some embodiments, the complex produces fewer indels in a target sequence having a 3′ end that is not directly adjacent to the canonical TAM sequence (5′-ATAAA-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold lower as compared to the amount of indels produced by a complex comprising IscB protein as provided by SEQ ID NO: 2 or 8 on the same target sequence. In some embodiments, indels are measured using high-throughput sequencing.

In some embodiments, the complex exhibits a decreased off-target activity as compared to the off-target activity of a complex comprising IscB protein as provided by SEQ ID NO: 2 or 8. In some embodiments, the off-target activity of the complex is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold decreased as compared to the off-target activity of a complex comprising IscB protein as provided by SEQ ID NO: 2 or 8. In some embodiments, the target sequence is in the genome of an organism. In some embodiments, the organism is a prokaryote. In some embodiments, the prokaryote is a bacterium. In some embodiments, the organism is a eukaryote. In some embodiments, the organism is a plant or fungus. In some embodiments, the organism is a vertebrate. In some embodiments, the vertebrate is a mammal. In some embodiments, the mammal is a human. In some embodiments, the organism is a cell. In some embodiments, the cell is a human cell.

In another aspect described herein is a method comprising contacting a nucleic acid with the fusion protein of any one of the embodiments described herein, and with a guide RNA, wherein the guide RNA is about 15-100 nucleotides long and comprises a sequence of at least 10 contiguous nucleotides that is complementary to a target sequence.

In another aspect described herein is a method comprising contacting a cell with the fusion protein of any one of the embodiments described herein, and with a guide RNA, wherein the guide RNA is about 15-100 nucleotides long and comprises a sequence of at least 10 contiguous nucleotides that is complementary to a target sequence.

In another aspect described herein is a method comprising contacting a nucleic acid with the complex of any one of the embodiments described herein.

In another aspect described herein is a method comprising contacting a cell with the complex of any one of the embodiments described herein.

In some embodiments in any of the aspects, the contacting is performed in vitro. In some other embodiments in any of the aspects, the contacting is performed in vivo.

In another aspect described herein is a method comprising administering to a subject the fusion protein of any one of the embodiments described herein, and a guide RNA, wherein the guide RNA is about 15-100 nucleotides long and comprises a sequence of at least 10 contiguous nucleotides that is complementary to a target sequence.

In another aspect described herein is method comprising administering to a subject the complex of any one of the embodiments described herein.

In some embodiments, the target sequence of the nucleic acid is a DNA sequence. In some embodiments, the 3′ end of the target sequence is not immediately adjacent to the canonical TAM sequence (5′-ATAAA-3′). In some embodiments, the target sequence comprises a sequence associated with a disease or disorder. In some embodiments, the target DNA sequence comprises a point mutation associated with a disease or disorder.

In some embodiments, the activity of the fusion protein, or the activity of the complex, results in a correction of the point mutation.

In some embodiments, the target DNA sequence comprises a T to C point mutation associated with a disease or disorder, and wherein the deamination of the mutant C base results in a sequence that is not associated with a disease or disorder. In some embodiments, the target DNA sequence encodes a protein and wherein the point mutation is in a codon and results in a change in the amino acid encoded by the mutant codon as compared to the wild-type codon. In some embodiments, the deamination of the mutant C results in a change of the amino acid encoded by the mutant codon. In some embodiments, the deamination of the mutant C results in the codon encoding the wild-type amino acid.

In some embodiments, the target DNA sequence comprises a G to A point mutation associated with a disease or disorder, and wherein the deamination of the mutant A base results in a sequence that is not associated with a disease or disorder. In some embodiments, the target DNA sequence encodes a protein and wherein the point mutation is in a codon and results in a change in the amino acid encoded by the mutant codon as compared to the wild-type codon. In some embodiments, the deamination of the mutant A results in a change of the amino acid encoded by the mutant codon. In some embodiments, the deamination of the mutant A results in the codon encoding the wild-type amino acid.

In some embodiments, the contacting is in vivo in a subject. In some embodiments, the subject has or has been diagnosed with a disease or disorder. In some embodiments, the disease or disorder is a proliferative disease, a genetic disease, a neoplastic disease, a metabolic disease, or a lysosomal storage disease.

In another aspect described herein is a kit comprising a nucleic acid construct, comprising: (a) a nucleic acid sequence encoding the fusion protein of any one of the embodiments described herein; and (b) a heterologous promoter that drives expression of the sequence of (a).

In another aspect described herein is a kit comprising a nucleic acid construct, comprising: (a) a nucleic acid sequence encoding the complex of any one of the embodiments described herein; and (b) a heterologous promoter that drives expression of the sequence of (a).

In some embodiments, the kit further comprises an expression construct encoding a guide RNA backbone, wherein the construct comprises a cloning site positioned to allow the cloning of a nucleic acid sequence identical or complementary to a target sequence into the guide RNA backbone.

In another aspect described herein is a polynucleotide encoding the fusion protein of any one of the embodiments described herein or the complex of any one of the embodiments described herein.

In another aspect described herein is a vector comprising a polynucleotide of any of the embodiments described herein.

In some embodiments, the vector comprises a heterologous promoter driving expression of the polynucleotide encoding the fusion protein or the polynucleotide encoding the complex.

In another aspect described herein is a method comprising contacting a cell with the vector of any of the embodiments described herein.

In some embodiments, the vector is transfected into the cell. In some embodiments, the vector is transfected into the cell using electroporation, heat shock, or a composition comprising a cationic lipid.

In another aspect described herein is a cell comprising the fusion protein of any one of the embodiments described herein, or a nucleic acid molecule encoding the fusion protein of any one of the embodiments described herein.

In another aspect described herein is a cell comprising the complex of any one of the embodiments described herein, or a nucleic acid molecule encoding the complex of any one of the embodiments described herein.

In another aspect described herein is a cell comprising the vector of any one of the embodiments described herein.

In some embodiments of any one of the aspects described herein, an IscB protein described herein has a non-canonical TAM specificity. Stated in another way, an IscB protein described herein can act on a target sequence that does not have the canonical TAM sequence 5′-ATAAA-3′ at its 3′ end.

In another aspect, provided herein is a method of modifying a target nucleic acid, the method comprising contacting an IscB (or a fusion protein) described herein and a guide nucleic acid with a target sequence.

In some embodiments, the target nucleic acid is in a cell and the method comprises administering to the cell a DNA and RNA targeting system or composition of any one of the preceding embodiments. In some embodiments, said modifying the target nucleic acid comprises cleaving the target nucleic acid. In some embodiments, said cleaving the target nucleic acid forms at least one 5′-overhang. In some embodiments, said modifying the target nucleic acid comprises deletion, insertion or change of a nucleotide in the target nucleic acid. In some embodiments, said modifying the target nucleic acid comprises editing (e.g., changing) one or more nucleobases; correcting or introducing a premature stop codon, disrupting a splice site; inserting or restoring a splice site, inserting a gene or gene fragment into the target nucleic acid, or a combination thereof.

In some embodiments of any of the aspects, the target nucleic acid is DNA. In some preferred embodiments, the target nucleic acid is genomic DNA, episomal DNA, viral DNA, or bacterial DNA. In some embodiments of any of the aspects, the target nucleic acid is double-stranded DNA. In some other embodiments of any of the aspects, the target nucleic acid is single-stranded DNA.

In some embodiments of any of the aspects, the target nucleic acid is RNA. In some other embodiments of any of the aspects, the target nucleic acid is double-stranded RNA. In some other embodiments, the target nucleic acid is single-stranded RNA. In some embodiments, the target nucleic acid is RNA selected from the group consisting of messenger RNA (mRNA), pre-mRNA, ribosomal RNA (rRNA), transfer RNA (tRNA), micro-RNA (miRNA), small nuclear RNA (snRNA), small nucleolar RNA (snoRNA), non-coding RNA (ncRNA), long non-coding RNA (lncRNA), and small cytoplasmatic RNA (scRNA).

In some embodiments of any of the aspects described herein, the target nucleic acid is a disease-associated nucleic acid sequence.

In another aspect, provided herein is an isolated nucleic acid modified by a method of anyone of the preceding embodiments.

In another aspect, provided herein is an isolated cell or progeny thereof comprising a nucleic acid modified by a method of anyone of the preceding embodiments

Generally, the IscB proteins and the guide nucleic acid described herein do not naturally occur together.

Described herein are DNA and RNA targeting enzymes and systems, that are useful for modifying target nucleic acid sequences. Accordingly in one aspect is a DNA and RNA targeting composition comprising an IscB protein of describe herein and a guide nucleic acid capable of binding with the IscB protein, wherein the guide nucleic acid comprises a guide sequence complementary to target sequence and capable of directing the IscB protein to a target polynucleotide.

In some embodiments in any of the aspects, the guide nucleic acid is from about 10 nucleotides to about 150 nucleotides in length, preferably 12 to 50 nt, more preferably 15 and 45 nt in length. In some embodiments, the guide nucleic acid further comprises an aptamer. In some embodiments in any of the aspects described herein, the guide nucleic acid further comprises an extension to add an RNA template.

In some embodiments of any of the aspects described herein, the target sequence comprises a target adjacent motif (TAM) sequence 3′ of the target sequence. In some embodiments of any of the aspects, the target polynucleotide is DNA. In some embodiments, the target sequence comprises a TAM sequence of ATR, where R is purine (e.g., G or A).

In some embodiments, the composition further comprises a functional domain associated with the IscB protein.

In some embodiments, the functional domain has transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, detectable activity, or any combination thereof. In some embodiments, the functional domain is covalently linked with the IscB protein, wherein the IscB is a fusion protein described herein.

In some embodiments, the composition further comprises a serine or tyrosine recombinase.

In some embodiments, the composition further comprises a homologous recombination donor template comprising a donor sequence for insertion into a target polynucleotide.

In some embodiments, a nuclease domain of the IscB protein is catalytically inactive. In some other embodiments, the nuclease domain has nickase activity or is engineered to have nickase activity.

In another aspect described herein is an engineered cell comprising the composition of any of the embodiments described herein.

In another aspect described herein is a method of modifying a target sequence in a cell, the method comprising administering a composition of any of the embodiments described herein to the cell.

In another aspect described herein is a method of modifying a target sequence in a cell, the method comprising administering to the cell a composition comprising an IscB protein and/or a guide nucleic acid of any of the embodiments described herein, wherein IscB protein and/or the guide nucleic acid are provided via one or more polynucleotides encoding the IscB and/or guide nucleic acid, and wherein the one or more polynucleotides are operably configured to express the IscB/or the guide nucleic acid. In some embodiments, the modifying comprises cleaving a DNA polynucleotide. In some preferred embodiments, the cleaving results in 5′ overhangs.

In another aspect described herein is a method of targeting a polynucleotide, comprising contacting a sample that comprises a target polynucleotide with the composition of any of the embodiments described herein, or one or more polynucleotides encoding one or more components of the composition of any of the embodiments described herein. In some embodiments, contacting results in modification of a gene product or modification of the amount or expression of a gene product. In some preferred embodiments, the target sequence of the polynucleotide is a disease-associated target sequence.

In another aspect described herein is a composition comprising: the IscB protein of any one of claims 1-32, wherein the IscB protein is catalytically inactive, a nucleotide deaminase associated with or otherwise capable of forming a complex with the IscB protein, and a guide nucleic acid capable of forming a complex with the IscB protein and directing site-specific binding at a target sequence. In some embodiments, the nucleotide deaminase is an adenosine deaminase or a cytidine deaminase.

In another aspect described herein is a cell or progeny thereof genetically engineered to express one or more components of the composition of any one of the embodiments described herein.

In another aspect described herein is a method of editing nucleic acids in target polynucleotides comprising delivering the composition of any of the embodiments described herein, or one or more polynucleotides encoding one or more components of the composition, to a cell or population of cells comprising the target sequence. In some embodiments, the target polynucleotides are target sequences within genomic DNA. In some preferred embodiments, the target polynucleotide is edited at one or more bases to introduce a G->A or C->T mutation.

In another aspect is an isolated cell or progeny thereof comprising one or more base edits made using the method of any one of the embodiments described herein.

In another aspect as described herein is a composition comprising: the IscB protein of any one of the embodiments described herein, wherein the IscB is catalytically inactive, a reverse transcriptase associated with or otherwise capable of forming a complex with the IscB protein, and a guide nucleic acid capable of forming a complex with the IscB protein and directing site-specific binding of the IscB to a target sequence, and wherein the guide nucleic further comprises a donor sequence for insertion into the target sequence.

In another aspect as described herein is a method of modifying target polynucleotides comprising delivering the composition of any of the embodiments described herein, or one or more polynucleotides encoding one or more components of the composition to a cell, wherein the complex directs the reverse transcriptase to the target sequence and the reverse transcriptase facilitates insertion of the donor sequence from the guide nucleic acid into the target sequence. In some embodiments of any of the aspects, insertion of the donor sequence: introduces one or more base edits; corrects or introduces a premature stop codon; disrupts a splice site; inserts or restores a splice site; inserts a gene or gene fragment at one or both alleles of the target polynucleotide; or a combination thereof.

In another aspect as described herein is an isolated cell or progeny thereof comprising the modifications made using the method of any of the embodiments described herein.

In another aspect as described herein is a composition comprising: the IscB protein of any one of the embodiments described herein; a non-LTR retrotransposon protein associated with or otherwise capable of forming a complex with the IscB protein; guide nucleic acid capable of forming a complex with the IscB protein and directing site-specific binding to a target sequence; and a donor construct comprising a donor polynucleotide for insertion to the target sequence and located between two binding elements capable of forming a complex with the non-LTR retrotransposon protein.

In some embodiments, the IscB protein is fused to the N-terminus of the non-LTR retrotransposon protein. In some embodiments, the IscB protein is engineered to have nickase activity. In some embodiments, the guide nucleic acid directs the fusion protein to a target sequence 5′ of the targeted insertion site, and wherein the IscB protein generates a double-strand break at the targeted insertion site. In some other embodiments, the guide nucleic acid directs the fusion protein to a target sequence 3′ of the targeted insertion site, and wherein the IscB protein generates a double-strand break at the targeted insertion site.

In some embodiments, the donor polynucleotide further comprises a polymerase processing element to facilitate 3′ end processing of the donor polynucleotide sequence. In some other embodiments, the donor polynucleotide further comprises a homology region to the target sequence on the 5′ end of the donor construct, the 3′ end of the donor construct, or both. In some embodiments of any of the aspects, the homology region is from 8 to 25 base pairs.

In another aspect as described herein is a method of modifying target polynucleotides comprising delivering the composition of any one of the embodiments described herein, or one or more polynucleotides encoding one or more components of the composition to a cell, wherein the complex directs the non-LTR retrotransposon protein to the target sequence and the non-LTR retrotransposon protein facilitates insertion of the donor polynucleotide sequence from the donor construct into the target polynucleotide.

In another aspect as described herein is an isolated cell or progeny thereof comprising the modifications made using the method of any of the embodiments described herein.

In one aspect, provided herein is a guide RNA (also referred to as a ωRNA) comprising a conserved portion of a ωRNA (parent ωRNA) comprising one or more stem-loop structures, wherein at least one of the stem-loop structures comprises a modification, and wherein a conserved portion of the ωRNA is shorter than the conserved portion of the parent ωRNA and is capable of binding with a transposon family encoded nuclease or a homolog or ortholog thereof.

It is noted that ωRNA is also referred to as omegaRNA herein and the terms ωRNA and omegaRNA are used interchangeably herein.

In some embodiments, the conserved region of the ωRNA comprises at least two stem-loop structures and wherein said modification is in a stem-loop structure near to the 5′-end (5′-stem-loop) of the conserved region ωRNA.

In some embodiments of any one of the aspects described herein, said modification comprises deletion of from about 3 to about 46 nucleotides from the 5′-stem-loop. In some embodiments of any one of the aspects described herein, said modification comprises deletion of 3, 7, 11, 15, 18, 22, 26, 30, 34, 37 or 41 nucleotides from the 5′-stem-loop. In some preferred embodiments of any one of the aspects described herein, said modification comprises deletion of 15, 18, 30, 37 or 41 nucleotides from the 5′-stem-loop. In some embodiments of any one of the aspects described herein, said modification comprises deletion of 8, 12, 16, 20, 23, 27, 31, 35, 39, 42 or 46 nucleotides from the 5′-stem-loop. In some preferred embodiments of any one of the aspects described herein, said modification comprises deletion of 20, 23, 35, 42 or 46 nucleotides from the 5′-stem-loop.

In some embodiments, nucleotides at position 12-57, 14-55, 16-54, 18-52, 20-50, 22-48, 24-46, 26-45, 28-43, 30-41 or 32-39 are deleted (counting from 5′-end, e.g., 5′-end of the conserved region of the parent ωRNA). In some embodiments, nucleotides at position 12-57, 14-55, 18-52, 24-46, or 26-45 are deleted (counting from 5′-end, e.g., 5′-end of the conserved region of the parent ωRNA). In some embodiments, the deleted nucleotides are replaced with a linker (e.g., a loop portion). In some embodiments, the linker is single-stranded nucleic acid comprising from about from about 4 nucleotides to about 15 nucleotides. In some embodiments, a loop portion of the 5′-stem-loop comprises from 4, 5 or 6 nucleotides. In some embodiments, a loop portion of the 5′-stem-loop comprises the nucleotide sequence GAAA or GAAAA.

In some embodiments, at least a portion of the 5′-stem-loop is within 45 nucleotides or within 25 nucleotides, counting from 5′-end, e.g., 5′-end of the conserved region of the parent ωRNA. In some embodiments, a stem portion of the 5′-stem-loop comprises a double-stranded region comprising from about 8 nucleotide base-pairs about 9 nucleotide base-pairs, about 10 nucleotide base-pairs, about 11 nucleotide base-pairs, about 12 nucleotide base-pairs, about 13 nucleotide base-pairs, about 14 nucleotide base-pairs, about 15 base pairs nucleotide base-pairs, about 16 base pairs nucleotide base-pairs, about 17 base pairs nucleotide base-pairs, about 18 base pairs nucleotide base-pairs, about 19 base pairs nucleotide base-pairs, about 20 base pairs nucleotide base-pairs, or about 25 base pairs nucleotide base-pairs. In some preferred embodiments, a stem portion of the 5′-stem-loop comprises a double-stranded region comprises 10 nucleotide base-pairs.

In some embodiments, the 5′-stem-loop comprises from about 20 nucleotides, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, or about 65 nucleotides. In some preferred embodiments, the 5′-stem-loop comprises 23, 24, 25, 26 or 27 nucleotides. In some embodiments of any of the aspects, the 5′-stem-loop comprises a nucleic acid modification, optionally selected from the group consisting of nucleobase modifications, sugar modifications, inter-sugar linkage modifications (modified internucleoside linkages), conjugates (e.g., ligands), and any combinations thereof.

In some embodiments, a stem (double-stranded) region of the 5′-stem-loop comprises a nucleic acid modification. In some other embodiments, a loop (single-stranded) region of the 5′-stem-loop comprises a nucleic acid modification.

In some embodiments, said nucleic acid modification is a modified nucleotide selected from the group consisting of 2′-O-methyl(2′-OMe) nucleotides, 2′-fluoro (2′-F) nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl(2′-MOE) nucleotides, 2′-O-allyl nucleotides, 2′-C-allyl ribose nucleotides, 2′-O-N-methylacetamido (2′-O-NMA) nucleotides, a 2′-O-dimethylaminoethoxyethyl(2′-O-DMAEOE) nucleotides, 2′-O-aminopropyl (2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a non-natural or modified nucleobase; or a combination thereof.

In some embodiments, a stem (double-stranded) region of the 5′-stem-loop comprises a 2′-O-methyl modified nucleotide. In some other embodiments, a stem (double-stranded) region of the 5′-stem-loop comprises a 2′-fluoro modified nucleotide. In some embodiments, a nucleotide in a stem (double-stranded) region of the 5′-stem-loop comprises a non-natural or modified nucleobase.

In some embodiments, a stem (double-stranded) region of the 5′-stem-loop comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

In some embodiments, a loop (single-stranded) region of the 5′-stem-loop comprises a 2′-O-methyl modified nucleotide. In some other embodiments, a loop (single-stranded) region of the 5′-stem-loop comprises a 2′-fluoro modified nucleotide.

In some embodiments, a nucleotide in a loop (single-stranded) region of the 5′-stem-loop comprises a non-natural or modified nucleobase.

In some embodiments, a loop (single-stranded) region of the 5′-stem-loop comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

In some embodiments, a stem (double-stranded) region of the 5′-stem-loop comprises a duplex stabilizing modification, optionally the duplex stabilizing modification is 2′-F nucleotide, 2′-OMe nucleotide, 2′-methoxyethyl nucleotide, 2,6-diaminopurine nucleotide, 5-methyl cytidine, N4-ethyl cytidine, 5-propynyl cytidine, 5-propynyl uridine, 5-hydroxybutynl-2′-deoxyuridine, 8-aza-7-deazaguanosine, a locked nucleic acid (LNA), and/or covalent cross-linking of two strands of the duplex.

In some embodiments, two nucleotides in the stem of the 5′-loop-stem are covalently linked to each other via a position other than 5′ and 3′.

In some embodiments, the conserved region of the ωRNA comprises at least two stem-loop structures and wherein said modification is in a stem-loop structure proximal to the 3′-end (3′-stem-loop) of the conserved region of the ωRNA.

In some embodiments, said modification comprises deletion of from about 1 to about 22 nucleotides from the 3′-stem-loop. In some preferred embodiments, said modification comprises deletion of 2, 6, 8, 10, 12, 16, or 20 nucleotides from the 3′-stem-loop. In some embodiments, said modification comprises deletion of 2, 10, 12, or 16 nucleotides from the 3′-stem-loop. In some embodiments, said modification comprises deletion of 2, 10, 12 or 16 nucleotides from the 5′-stem-loop. In some embodiments, said modification comprises deletion of 2, 10, 12, or 16 nucleotides from the 3′-stem-loop.

In some embodiments, nucleotides at position 159-172, 160-171, 161-170, 163-168, 158-173, 156-175 or 154-177 are deleted (counting from 5′-end, e.g., of the parent ωRNA). In some other embodiments, nucleotides at position 159-172, 163-168, 158-173, or 156-175 are deleted (counting from 5′-end, e.g., of the parent ωRNA).

In some embodiments, the deleted nucleotides in the 3′-stem-loop are replaced with a linker (e.g., a loop portion). In some embodiments, the linker is a single-stranded nucleic acid comprising from about from about 4 nucleotides to about 15 nucleotides.

In some embodiments, a loop portion of the 5′-stem-loop comprises from 4, 5 or 6 nucleotides. In some embodiments, a loop portion of the 5′-stem-loop comprises the nucleotide sequence GAAA or GAAAA.

In some embodiments, at least a portion of the 3′-stem-loop is within 50 nucleotides, counting from 3′-end of the modified ωRNA. In some other embodiments, at least a portion of the stem of the 5′-stem-loop is within 45 nucleotides, counting from 3′-end of the modified RNA.

In some embodiments, a stem portion of the 3′-stem-loop comprises a double-stranded region comprising from about 3 nucleotide base-pairs to about 10 nucleotide base-pairs. In some other embodiments, a stem portion of the 3′-stem-loop comprises a double-stranded region comprising about 3 nucleotide base-pairs, about 4 nucleotide base-pairs, about 5 nucleotide base-pairs, about 6 nucleotide base-pairs, about 7 nucleotide base-pairs, or about 8 nucleotide base-pairs. In some preferred embodiments, a stem portion of the 3′-stem-loop comprises a double-stranded region comprising about 3 nucleotide base-pairs, about 4 nucleotide base-pairs, about 5 nucleotide base-pairs, or about 6 nucleotide base-pairs.

In some embodiments, a stem portion of the 5′-stem-loop comprises a double-stranded region comprises 3 or 4 nucleotide base-pairs.

In some embodiments, the 3′-stem-loop comprises from about 8 nucleotides, about 9, about 10, about 11, about 12, about 13, about 14, about 15, about 16, about 17, about 18, about 19, about 20, about 21, about 22, about 23, about 24, or about 25 nucleotides. In some preferred embodiments, the 3′-stem-loop comprises 9, 10, 11, 12, 13, 14 or 15 nucleotides. In some embodiments of any of the aspects described herein, the 3′-stem-loop comprises a nucleic acid modification, optionally selected from the group consisting of nucleobase modifications, sugar modifications, inter-sugar linkage modifications (modified internucleoside linkages), conjugates (e.g., ligands), and any combinations thereof.

In some embodiments, a stem (double-stranded) region of the 3′-stem-loop comprises a nucleic acid modification. In some embodiments, a loop (single-stranded) region of the 3′-stem-loop comprises a nucleic acid modification. In some embodiments of any of the aspects described herein, said nucleic acid modification is a modified nucleotide selected from the group consisting of 2′-O-methyl(2′-OMe) nucleotides, 2′-fluoro (2′-F) nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl(2′-MOE) nucleotides, 2′-O-allyl nucleotides, 2′-C-allyl ribose nucleotides, 2′-O-N-methylacetamido (2′-O-NMA) nucleotides, a 2′-O-dimethylaminoethoxyethyl(2′-O-DMAEOE) nucleotides, 2′-O-aminopropyl(2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a non-natural or modified nucleobase; or a combination thereof.

In some embodiments, a stem (double-stranded) region of the 3′-stem-loop comprises a 2′-O-methyl modified nucleotide. In some other embodiments, a stem (double-stranded) region of the 3′-stem-loop comprises a 2′-fluoro modified nucleotide. In some embodiments, a nucleotide in a stem (double-stranded) region of the 5′-stem-loop comprises a non-natural or modified nucleobase. In some embodiments, a stem (double-stranded) region of the 3′-stem-loop comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂-O-5′), oxime, methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

In some embodiments, a loop (single-stranded) region of the 5′-stem-loop comprises a 2′-O-methyl modified nucleotide. In some other embodiments, a loop (single-stranded) region of the 5′-stem-loop comprises a 2′-fluoro modified nucleotide. In some embodiments, a nucleotide in a loop (single-stranded) region of the 3′-stem-loop comprises a non-natural or modified nucleobase. In some embodiments, a loop (single-stranded) region of the 5′-stem-loop comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂-0-5′), oxime, methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS). In some embodiments, two nucleotides in the stem of the 5′-loop-stem are covalently linked to each other via a position other than 5′ and 3′.

In some embodiments, the conserved region of the ωRNA comprises at least three stem-loop structures. In some embodiments, at least one (e.g., 1, 2, 3, 4, 5, 6, 7 or 8) nucleotide in a loop region of a stem-loop forms a base-pair with a nucleotide outside said stem-loop. At least one (e.g., one or both) member of the base-pair comprises a modification capable of enhancing base-pairing. In some embodiments, the base-pair nucleotide in the loop comprises the modification capable of enhancing base-pairing. In some other embodiments, the base-pair nucleotide outside said stem-loop comprises the modification capable of enhancing base-pairing.

In some embodiments, said modification capable of enhancing base-pairing is 2′-F modified nucleotide, 2′-OMe modified nucleotide, 2′-methoxyethyl modified nucleotide, 2,6-diaminopurine nucleotide, 5-methyl cytidine, N4-ethyl cytidine, 5-propynyl cytidine, 5-propynyl uridine, 5-hydroxybutynl-2′-deoxyuridine, 8-aza-7-deazaguanosine, or a locked nucleic acid (LNA). In some embodiments, said modification capable of enhancing base-pairing is in at an internal (i.e., not a 5′-stem-loop or 3′-stem-loop) stem-loop of the ωRNA. In some preferred embodiments, said modification capable of enhancing base-pairing is in a loop (single-stranded) region of an internal (i.e., not a 5′-stem-loop or 3′-stem-loop) stem-loop of the ωRNA.

In some embodiments, the ωRNA (e.g., a conserved region) is from about 50 to about 90 nucleotides shorter than the parent ωRNA. In some embodiments, the ωRNA (e.g., a conserved region) is from about 55 to about 85 nucleotides shorter than the parent ωRNA. In some embodiments, the ωRNA (e.g., a conserved region) is about 60, about 61, about 62, about 63, about 64, about 65, about 66, about 67, about 68, about 69, about 70, about 71, about 72, about 73, about 74, about 75, about 76, about 77, about 78, about 79, or about 80 nucleotides shorter than the parent ωRNA. In some embodiments, the ωRNA (e.g., a conserved region) is from about 130 nucleotides to about 190 nucleotides in length. In some embodiments, the ωRNA (e.g., a conserved region) is from about 135 nucleotides to about 180 nucleotides in length. In some embodiments, the ωRNA (e.g., a conserved region) is from about 140 nucleotides to about 170 nucleotides in length. In some embodiments, the ωRNA (e.g., a conserved region) is from about 140, about 141, about 142, about 143, about 144, about 145, about 146, about 147, about 148, about 149, about 150, about 151, about 152, about 153, about 1454, about 155, about 156, about 157, about 158, about 159, or about 160 nucleotides in length.

In some embodiments, the ωRNA comprises a 5′ end modification. In some other embodiments, the ωRNA comprises a 3′ end modification. In some embodiments, the ωRNA comprises a 5′ end modification and a 3′ end modification. In some preferred embodiments, the 5′ and/or 3′ end modification comprises a protective end modification where the protective end modification is a modified nucleotide selected from 2′-O-methyl(2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, a 3′->3′ linkage between nucleotides, an inverted abasic modified nucleotide, or combinations thereof.

In some embodiments, the 3′ end modification comprises any one of: a modification of any one or more of the last 7, 6, 5, 4, 3, 2, or 1 nucleotides; one modified nucleotide; two modified nucleotides; three modified nucleotides; four modified nucleotides; five modified nucleotides; six modified nucleotides; or seven modified nucleotides. In some other embodiments, the 3′ end modification comprises one or more of: a modified internucleoside linkage (e.g., PS, imidp or MMI linkage) between nucleotides; a 2′-OMe nucleotide; a 2′-O-MOE nucleotide; a 2′-F nucleotide; a LNA nucleotide; a 3′->3′ linkage between nucleotides; an inverted abasic nucleotide; and a combination of one or more of (i.)-(vii.). In some preferred embodiments, the 3′ end modification comprises one or more of: a modified internucleoside linkage (e.g., PS, imidp or MMI linkage) between the last and second to last nucleotides; a modified internucleoside linkage (e.g., PS, imidp or MMI linkage) between the last and second to last nucleotide and a PS linkage between the second and third to last nucleotides; modified internucleoside linkages (e.g., PS and/or MMI linkage) between any one or more of the last four nucleotides; modified internucleoside linkages (e.g., PS and/or MMI linkage) between any one or more of the last five nucleotides; or modified internucleoside linkages (e.g., PS and/or MMI linkage) between any one or more of the last 2, 3, 4, 5, 6, 7, 8, 9, or 10 nucleotides.

In some embodiments, the 3′ end modification comprises at least one 2′-F nucleotide, 2′-OMe nucleotide, 2′-O-MOE nucleotide, inverted abasic nucleotide or a 3′->3′ internucleoside linkage. In some embodiments, the 3′ end modification comprises: a modification of one or more of the last 1-7 nucleotides, wherein the modification is a modified internucleoside linkages (e.g., PS and/or MMI linkage), inverted abasic nucleotide, a 3′->3′ internucleoside linkage, 2′-OMe, 2′-O-MOE, 2′-F, LNA, or a combination thereof; a modification to the last nucleotide with 2′-OMe, 2′-O-MOE, 2′-F, LNA, or combinations thereof, and an optional one or two modified internucleoside linkages (e.g., PS and/or MMI linkage) to the next nucleotide; a modification to the last and/or second to last nucleotide with 2′-OMe, 2′-O-MOE, 2′-F, LNA, or combinations thereof, and optionally one or more modified internucleoside linkages (e.g., PS and/or MMI linkage); iv. a modification to the last, second to last, and/or third to last nucleotides with 2′-OMe, 2′-O-moe, 2′-F, or combinations thereof, and optionally one or more modified internucleoside linkages (e.g., PS and/or MMI linkage); a modification to the last, second to last, third to last, and/or fourth to last nucleotides with 2′-OMe, 2′-O-MOE, 2′-F, LNA or combinations thereof, and optionally one or more modified internucleoside linkages (e.g., PS and/or MMI linkage); or a modification to the last, second to last, third to last, fourth to last, and/or fifth to last nucleotides with 2′-OMe, 2′-O-MOE, 2′-F, LNA, or combinations thereof, and optionally one or more modified internucleoside linkages (e.g., PS and/or MMI linkage).

In some embodiments, the 3′ end modification comprises any one or more of: a 2′-OMe modified nucleotide at the last position, three consecutive 2′-O-MOE modified nucleotides immediately 5′ to the 2′-OMe modified nucleotide, and three consecutive PS linkages between the last three nucleotides; five consecutive 2′-OMe modified nucleotides from the 3′ end of the 3′ terminus, and three PS linkages between the last three nucleotides; an inverted abasic modified nucleotide at the last position; an inverted abasic modified nucleotide at the last position, and three consecutive 2′-OMe modified nucleotides at the last three positions; 15 consecutive 2′-OMe modified nucleotides from the 3′ end, five consecutive 2′-F modified nucleotides immediately 5′ to the 2′-OMe modified nucleotides, and three PS linkages between the last three nucleotides; alternating 2′-OMe modified nucleotides and 2′-F modified nucleotides at the last 20 nucleotides, and three PS linkages between the last three nucleotides; two or three consecutive 2′-OMe modified nucleotides, and three PS linkages between the last three nucleotides; one PS linkage between the last and next to last nucleotides; and 15 or 20 consecutive 2′-OMe modified nucleotides, and three PS linkages between the last three nucleotides.

In some embodiments, the 5′ end modification comprises any one or more of: one modified nucleotide; two modified nucleotides; three modified nucleotides; four modified nucleotides; five modified nucleotides; six modified nucleotides; and seven modified nucleotides. In some embodiments, the 5′ end modification comprises any one or more of: a modification of between 1 and 7, between 1 and 5, between 1 and 4, between 1 and 3, or between 1 and 2 nucleotides. In some embodiments, the 5′ end modification comprises any one or more of: (i) a modified internucleoside linkage (e.g., a phosphorothioate and/or MMI linkage) between nucleotides; (ii) a 2′-OMe nucleotide; (iii) a 2′-O-MOE nucleotide; (iv) a 2′-F nucleotide; (v) a LNA; (vi) a 3′->3′ linkage; (vii) an inverted abasic modified nucleotide; (viii) a deoxyribonucleotide; (ix) an inosine; and (x) combinations of one or more of (i)-(ix).

In some other embodiments, the 5′ end modification comprises: 1, 2, 3, 4, 5, 6, and/or 7 modified internucleoside (e.g., a phosphorothioate and/or MMI) linkages between nucleotides; or about 1-2, 1-3, 1-4, 1-5, 1-6, or 1-7 modified internucleoside (e.g., a phosphorothioate and/or MMI) linkages between nucleotides.

In some preferred embodiments, the 5′ end modification comprises: there is one modified internucleoside (e.g., a phosphorothioate and/or MMI) linkage, and the linkage is between nucleotides 1 and 2; there are two modified internucleoside (e.g., a phosphorothioate and/or MMI) linkages, and the linkages are between nucleotides 1 and 2, and 2 and 3; there are modified internucleoside (e.g., a phosphorothioate and/or MMI) linkages between any one or more of nucleotides 1 and 2, 2 and 3, and 3 and 4; there are modified internucleoside (e.g., a phosphorothioate and/or MMI) linkages between any one or more of nucleotides 1 and 2, 2 and 3, 3 and 4, and 4 and 5; there are modified internucleoside (e.g., a phosphorothioate and/or MMI) linkages between any one or more of nucleotides 1 and 2, 2 and 3, 3 and 4, 4 and 5, and 5 and 6; there are modified internucleoside (e.g., a phosphorothioate and/or MMI) linkages between any one or more of nucleotides 1 and 2, 2 and 3, 3 and 4, 4 and 5, 5 and 6, and 6 and 7; or there are modified internucleoside (e.g., a phosphorothioate and/or MMI) linkages between any one or more of nucleotides 1 and 2, 2 and 3, 3 and 4, 4 and 5, 5 and 6, 6 and 7, and 7 and 8. In some embodiments, the 5′ end modification comprises at least one 2′-OMe, 2′-O-MOE, inverted abasic, or 2′-F modified nucleotide.

In some embodiments, the ωRNA further comprises a guide region capable of hybridizing with a target nucleic acid. In some embodiments, the ωRNA is hybridized with a second nucleic acid strand. In some embodiments, the second strand comprises a guide region comprising a sequence capable of hybridizing with a target nucleic acid. In some embodiments, the guide region is from about 15 to about 30 nucleotides in length. In some embodiments, the guide region is from about 18 to about 25 nucleotides in length. In some embodiments, the guide region is from 18, 19, 20, 21, 22 or 23 nucleotides in length. In some embodiments, the guide region is at 5′-end of the ωRNA. In some embodiments, the guide region is at 3′-end of the ωRNA.

In some embodiments, the guide region comprises a nucleic acid modification, optionally selected from the group consisting of nucleobase modifications, sugar modifications, inter-sugar linkage modifications (modified internucleoside linkages), conjugates (e.g., ligands), and any combinations thereof.

In some embodiments, the guide region comprises: a modification of one or more of nucleotides 1-7, wherein the modification is a modified internucleoside (e.g., phosphorothioate and/or MMI) linkage, 3′->3′ internucleoside linkage, inverted abasic nucleotide, 2′-OMe, 2′-O-MOE, 2′-F, 2′-H (a deoxyribonucleotide), an inosine, and/or combinations thereof; a modification to the first nucleotide of the guide region with 2′-OMe, 2′-O-MOE, 2′-F, 2′-H, an inosine, or combinations thereof, and an optional modified internucleoside (e.g., phosphorothioate and/or MMI) linkage to the next nucleotide; a modification to the first and/or second nucleotide of the guide region with 2′-OMe, 2′-O-MOE, 2′-F, 2′-H, an inosine, or combinations thereof, and optionally one or more modified internucleoside (e.g., phosphorothioate and/or MMI) linkages; a modification to the first, second, and/or third nucleotides of the guide region with 2′-OMe, 2′-O-MOE 2′-F, 2′-H, an inosine, or combinations thereof, and optionally one or more modified internucleoside (e.g., phosphorothioate and/or MMI) linkages; a modification to the first, second, third, and/or fourth nucleotides of the guide region with 2′-OMe, 2′-O-MOE, 2′-F, 2′-H, an inosine, or combinations thereof, and optionally one or more modified internucleoside (e.g., phosphorothioate and/or MMI) linkages; or a modification to the first, second, third, fourth, and/or fifth nucleotides of the guide region with 2′-OMe, 2′-O-MOE, 2′-F, 2′-H, an inosine, or combinations thereof, and optionally one or more modified internucleoside (e.g., phosphorothioate and/or MMI) linkages.

In some embodiments, the ωRNA comprises any one or more of: 2′-OMe nucleotides at nucleotides 1, 2, and 3 of the guide region; 2′-OMe nucleotides at nucleotides 1, 2, and 3 of the guide region and modified internucleoside (e.g., phosphorothioate and/or MMI) linkages between nucleotides 1 and 2, 2 and 3, and 3 and 4 of the guide region; 2′-OMe nucleotides at nucleotides 1, 2, 3, 4, and 5 of the guide region; 2′-OMe nucleotides at nucleotides 1, 2, 3, 4, and 5 of the guide region and modified internucleoside (e.g., phosphorothioate and/or MMI) linkages between nucleotides 1 and 2, 2 and 3, 3 and 4, 4 and 5, and 5 and 6 of the guide region; 2′-O-MOE modified nucleotides at nucleotides 1, 2, and 3 of the guide region; 2′-O-MOE modified nucleotides at nucleotides 1, 2, and 3 of the guide region and modified internucleoside (e.g., phosphorothioate and/or MMI) linkages between nucleotides 1 and 2, 2 and 3, and 3 and 4 of the guide region; an inverted abasic nucleotide at nucleotide 1 of the guide region; an inverted abasic nucleotide at nucleotide 1 of the guide region and 2′-OMe nucleotides at nucleotides 1, 2, and 3 of the guide region; and an inverted abasic nucleotide at nucleotide 1 of the guide region, 2′-OMe nucleotides at nucleotides 1, 2, and 3 of the guide region, and modified internucleoside (e.g., phosphorothioate and/or MMI) linkages between nucleotides 1 and 2, 2 and 3, 3 and 4, 4 and 5, and 5 and 6 of the variable region.

In some embodiments, the ωRNA comprises one of more (e.g., 1, 2, 3, 4, 5, 6, 7, 8, 9, 10 or more including all) modified pyrimidine-A dinucleotide (modified YA site). In some embodiments, the ωRNA comprises at least one modified YA site in the guide region. In some embodiments, the ωRNA comprises at least one modified YA site in a conserved region of the ωRNA. In some embodiments, the modified YA site comprises a 2′-fluoro nucleotide, optionally the pyrimidine of the YA site; a 2′-H nucleotide, optionally the pyrimidine of the YA site; a 2′-OMe nucleotide, optionally the pyrimidine of the YA site; a PS linkage between the nucleotides; or a combination thereof. In some embodiments, the ωRNA comprises: a modified YA site at 1, 2, 3, 4, or 5 guide region YA sites; a modified YA site at 1, 2, 3, 4, or 5 guide region YA sites, wherein the modification of at least one guide region YA site is different from any 5′ end modification of the ωRNA; a modified YA site at one or more guide region YA sites that are at or after nucleotide 8 from the 5′ end of the 5′ terminus; a modified YA site at one or more guide region YA sites that are within nucleotides 5-end, 6-end, 7-end, 8-end, 9-end, or 10-end from the 5′ end of the 5′ terminus; a modified YA site at one or more guide region YA sites that are within 17, 16, 15, 14, 13, 12, 11, 10, or 9 nucleotides of the 3′ terminal nucleotide of the guide region; a modified YA site at a guide region YA site other than a 5′ end modification; or a modified YA site at a guide region YA site, wherein the modification of the guide region YA site comprises a modification at least one nucleotide located 5′ of the guide region YA site does not comprise.

In some embodiments, the ωRNA comprises: a modified YA site at two or more guide region YA sites that are at or after nucleotide 8 from the 5′-end; a modified YA site at two or more guide region YA sites that are within nucleotides 5-end, 6-end, 7-end, 8-end, 9-end, or 10-end from the 5′ end; a modified YA site at two or more guide region YA sites that are within 17, 16, 15, 14, 13, 12, 11, 10, or 9 nucleotides of the 3′ terminal nucleotide of the guide region; a modified YA site at two or more guide region YA sites other than a 5′ end modification; or a modified YA site at a two or more guide region YA sites, wherein the modifications of the guide region YA sites comprise a modification at least one nucleotide located 5′ of the guide region YA site does not comprise.

In some other embodiments, the ωRNA comprises: a modified YA site at three or more guide region YA sites that are at or after nucleotide 8 from the 5′ end of the 5′ terminus; a modified YA site at three or more guide region YA sites that are within nucleotides 5-end, 6-end, 7-end, 8-end, 9-end, or 10-end from the 5′ end of the 5′ terminus; a modified YA site at three or more guide region YA sites that are within 17, 16, 15, 14, 13, 12, 11, 10, or 9 nucleotides of the 3′ terminal nucleotide of the guide region; a modified YA site at three or more guide region YA sites other than a 5′ end modification; or a modified YA site at a three or more guide region YA sites, wherein the modifications of the guide region YA sites comprise a modification at least one nucleotide located 5′ of the guide region YA site does not comprise.

In some embodiments, the ωRNA retains at least 75% of the activity of the parent ωRNA.

In some embodiments of any of the aspects described herein, the nuclease is a transposon family nuclease. In some embodiments, the nuclease is a IS200/IS605 transposon family encoded nuclease or a homolog or ortholog thereof. In some embodiments, the nuclease is ISC (insertion sequences Cas9-like), TnpB, or a homolog or ortholog thereof. In some embodiments, the nuclease is IscB nuclease, IsrB nuclease, TnpB nuclease, or a homolog or ortholog thereof, optionally, the nuclease is IscB nuclease or a homolog or ortholog thereof. In some embodiments, the nuclease is modified nuclease.

In some embodiments, the ωRNA lacks from 1 to about 20 nucleotides present at 3′-end of the conserved portion of the parent ωRNA.

In another aspect is a composition comprising a ωRNA described herein or a polynucleotide encoding the same.

In some embodiments, the composition is in form of a lipid nanoparticles (LNP) or a protein nanoparticles (PNP).

In another aspect is a pharmaceutical composition comprising a ωRNA described herein or a composition of any one of the preceding embodiments.

In another aspect is a DNA and RNA targeting system comprising: (i) a protein effector polypeptide or a polynucleotide encoding the protein effector polypeptide, optionally, the protein effector polypeptide has nuclease (e.g., endonuclease activity); and (ii) a ωRNA of any one of the preceding embodiments or a polynucleotide encoding the ωRNA.

In another aspect is a DNA and RNA targeting composition comprising: (i) a protein effector polypeptide or a polynucleotide encoding the protein effector polypeptide, optionally, the protein effector polypeptide has nuclease (e.g., endonuclease activity); and (ii) a ωRNA of any one of the preceding embodiments or a polynucleotide encoding the ωRNA.

In some embodiments, the protein effector polypeptide comprises a RuvC domain, and/or an HNH domain. In some embodiments, the protein effector polypeptide comprises: (i) a split Ruv-C nuclease domain comprising RuvC-I, RuvC-II, and RuvC-III subdomains; and/or (ii) an HNH domain. In some embodiments, the protein effector polypeptide comprises a PLMP domain and optionally a conserved C-terminal Y domain.

In some embodiments, the HNH domain is located in the RuvC domain (e.g., the HNH domain is located between RuvC-II and RuvC-III subdomains). In some embodiments, the protein effector polypeptide comprises a HNH domain but no RuvC domain. In some other embodiments, the protein effector polypeptide comprises a RuvC (e.g., split RuvC) domain but no HNH domain. In some embodiments, the protein effector polypeptide further comprises a REC domain. In some embodiments, the REC domain is downstream from the RuvC or HNH domain, however in some other embodiments, the REC domain is upstream from the RuvC or HNH domain. In some embodiments of any of the aspects described herein, the protein effector polypeptide comprises from about 170 to about 1000 amino acids.

In some embodiments, the ωRNA comprises a guide sequence comprising a nucleotide sequence substantially complementary to at least a portion of a target nucleic acid (target sequence). In some embodiments, the target nucleic acid comprises a target adjacent motif (TAM) sequence 3′ of the target sequence. In some embodiments, the TAM comprises the sequence NAC, where N is A, C, G, or T.

In some embodiments, the DNA and RNA targeting system or composition further comprises a functional domain associated with the protein effector polypeptide. In some embodiments, the functional domain has transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, detectable activity, or any combination thereof. In some preferred embodiments, the DNA and RNA targeting system or composition further comprises a serine or tyrosine recombinase.

In some embodiments, the DNA and RNA targeting system or composition further comprises a homologous recombination donor template comprising a donor sequence for insertion into a target polynucleotide.

In another aspect is an engineered cell comprising a DNA and RNA targeting system or composition of any one of the preceding embodiments.

In another aspect is a method of modifying a target nucleic acid, the method comprising contacting a DNA and RNA targeting system or composition of any one of the preceding embodiments.

In some embodiments, the target nucleic acid is in a cell and the method comprises administering to the cell a DNA and RNA targeting system or composition of any one of the preceding embodiments. In some embodiments, said modifying the target nucleic acid comprises cleaving the target nucleic acid. In some embodiments, said cleaving the target nucleic acid forms at least one 5′-overhang. In some embodiments, said modifying the target nucleic acid comprises deletion, insertion or change of a nucleotide in the target nucleic acid. In some embodiments, said modifying the target nucleic acid comprises editing (e.g., changing) one or more nucleobases; correcting or introducing a premature stop codon, disrupting a splice site; inserting or restoring a splice site, inserting a gene or gene fragment into the target nucleic acid, or a combination thereof.

In some embodiments of any of the aspects, the target nucleic acid is DNA. In some preferred embodiments, the target nucleic acid is genomic DNA, episomal DNA, viral DNA, or bacterial DNA. In some embodiments of any of the aspects, the target nucleic acid is double-stranded DNA. In some other embodiments of any of the aspects, the target nucleic acid is single-stranded DNA.

In some embodiments of any of the aspects, the target nucleic acid is RNA. In some other embodiments of any of the aspects, the target nucleic acid is double-stranded RNA. In some other embodiments, the target nucleic acid is single-stranded RNA. In some embodiments, the target nucleic acid is RNA selected from the group consisting of messenger RNA (mRNA), pre-mRNA, ribosomal RNA (rRNA), transfer RNA (tRNA), micro-RNA (miRNA), small nuclear RNA (snRNA), small nucleolar RNA (snoRNA), non-coding RNA (ncRNA), long non-coding RNA (lncRNA), and small cytoplasmatic RNA (scRNA).

In some embodiments of any of the aspects described herein, the target nucleic acid is a disease-associated nucleic acid sequence.

In another aspect is an isolated nucleic acid modified by a method of anyone of the preceding embodiments.

In another aspect is an isolated cell or progeny thereof comprising a nucleic acid modified by a method of anyone of the preceding embodiments

Generally, the protein effector polypeptide and the guide nucleic acid described herein do not naturally occur together.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 examines the editing efficiency of engineered EboIscB variants over 48 genomic targets. The median editing efficiency over each variant is labeled. Wild-type EboIscB polypeptide is essentially pEDIT070.

FIG. 2A shows the engineering of TAMs either through the use of motif swaps or single mutants. FIG. 2B shows the validation of the human cell TAMs by examining the editing efficiency using either ATAAA targets or GTAAA targets. Figure discloses SEQ ID NOS 432, 47, 14-15, 18, 433, 32 and 35, respectively, in order of columns.

FIGS. 3A-3C show strategies for engineering gRNA for efficiency and manufacturability. FIG. 3A examines the editing efficiency through the guide RNA: nuclease plasmid molar ratio. FIG. 3B shows nucleotides targeted for a shortened gRNA. FIG. 3C shows for three different targets that compact gRNA improves efficiency. Figure discloses SEQ ID NO: 10.

FIGS. 4A-4C examine secondary structure of OgeuIscB OmegaRNA and the predicted structure of WT and engineered EboIscB OmegaRNA. FIG. 4A depicts secondary folding structure of Wild-type OmegaRNA for OgeuIscB. A 20-nt variable region in each of the 5′-end of the OmegaRNA is labeled as “5′ N20”. For more details about the OgeuIscB polypeptide and associated OmegaRNA, see e.g., Kato et al. “Structure of the IscB-ωRNA ribonucleoprotein complex, the likely ancestor of CRISPR-Cas9,” Nature Communications volume 13, Article number: 6719 (2022); and Altae-Tan et al., “The widespread IS200/IS605 transposon family encodes diverse programmable RNA-guided endonucleases,” Science 2021 October, 374 (6563): 57-65, the contents of which are incorporated herein by reference in their entirety; the contents of each of which are incorporated herein by reference in their entireties. FIG. 4B-4C show schematics of wild-type (FIG. 4B) or engineered M71 (FIG. 4C) OmegaRNA of EboIscB. Wild-type EboIscB polypeptide is essentially pEDIT070. Figure discloses SEQ ID NOS 434-437, respectively, in order of appearance.

### DETAILED DESCRIPTION

The section headings used herein are for organizational purposes only and are not to be construed as limiting the subject matter described.

### Protein Effector Polypeptides

Generally, the protein effector polypeptide has nuclease (e.g., endonuclease) activity. For example, the protein effector polypeptide described herein are RNase and have nuclease activity on RNA. In another example, the protein effector polypeptide described herein are DNase and have nuclease activity on DNA.

In some embodiments, the protein effector polypeptide is a transposon family encoded nuclease or a homolog or ortholog thereof. For example, the protein effector polypeptide is a IS200/IS605 transposon family encoded nuclease or a homolog or ortholog thereof. In some embodiment, the protein effector polypeptide is IscB, Isrb, TnpB, or a homolog or ortholog thereof. In some embodiments, the protein effector polypeptide is a modified IS200/IS605 transposon family encoded nuclease.

In some embodiments, the protein effector polypeptide is a Class 2 CRISPR-Cas effector. Class 2 effectors contain an isolated CRISPR-Cas effector protein having a RuvC domain and an isolated RNA guide, comprising a tracrRNA and a crRNA, wherein the crRNA includes a spacer sequence that is complementary to a target nucleic acid sequence such as an RNA sequence. In some embodiments, the Class 2 CRISPR-Cas effector is derived from an uncultivated microorganism.

In some embodiments, the protein effector polypeptide is a class 2, type II Cas endonuclease. In Class 2 Type II systems there are two strands of RNA that are part of the CRISPR-Cas system: a CRISPR RNA (crRNA) and a transactivating CRISPR RNA (tracrRNA). The tracrRNA hybridizes to a complementary region of pre-crRNA facilitating maturation of the pre-crRNA to crRNA by an RNase III enzyme. The duplex formed by the tracrRNA and crRNA is recognized by, and associates with a protein, e.g., Cas9, which is directed to a target nucleic acid by a sequence of the crRNA that is complementary to, and hybridizes with, a sequence in the target nucleic acid. In some embodiments, the class 2, type II Cas endonuclease is not a Cas9 endonuclease, a Cas 14 endonuclease, a Cas 12a endonuclease, a Cas 12b endonuclease, a Cas 12c endonuclease, a Cas 12d endonuclease, a Cas12e endonuclease, a Cas13a endonuclease, a Cas 13b endonuclease, a Cas13c endonuclease, or a Cas 13d endonuclease. In some embodiments, the class 2, type II Cas endonuclease is derived from an uncultivated microorganism.

Class 2, type II Cas endonuclease comprise a RuvC domain and/or HNH domain. Accordingly, in some embodiments, the protein effector polypeptide described herein comprises one or more nuclease domains, such as RuvC domain and/or HNH domain. In some embodiments, the protein effector polypeptide comprises at least two nuclease domains, where one of the nuclease domains is responsible for the cleavage one strand of a double-stranded target nucleic acid and the other nuclease domains is responsible for cleavage of the other strand of the double-stranded target nucleic acid.

In some embodiments of any one of the aspects described herein, the protein effector polypeptide comprises a RuvC domain and/or a HNH domain. Without wishing to be bound by a theory, the RuvC domain is responsible for the cleavage of the target DNA strand, while the HNH domain is responsible for cleavage of the displaced DNA strand. Generally, the protein effector polypeptide comprises at least one nuclease domain, i.e., the protein effector polypeptide comprises a RuvC domain or a HNH domain. In some embodiments, the protein effector polypeptide protein comprises at least two nuclease domains. For example, the protein effector polypeptide comprises at least two RuvC domains, or at least two HNH domains, or at least one RuvC domain and at least one HNH domain. In some preferred embodiments, the protein effector polypeptide comprises at least one RuvC domain and at least one HNH domain.

In some embodiments, the protein effector polypeptide is chimeric. For example, the protein effector polypeptide comprises a first fragment and a second fragment, and wherein the fragments can be of protein effector polypeptide orthologs of organisms of a genus or of species. In some embodiments, the protein effector polypeptide comprises a first fragment and a second fragment, and wherein the first and second fragments are from protein effector polypeptide orthologs of different species.

In some embodiments, the protein effector polypeptide is an modified IscB comprising the sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with any one of the sequences:

(SEQ ID NO: 1; pEDIT070)
MRYVYVLDVDGKPLMPTCRFGKVRRMLKSGQAKAVDTLPFTIQLTYKPRTRI
LQPVTLGQDPGRTNIGMAAVRFDGKELGRFHCITRNKEIPKLMADRMAARKA
SRRGERLARKRLARKLHTTAKHLNGRILPGCSEPIAVKDIINTESRFNNRLRPE
GWLTPTATQLLRTHINLFRKLSGILPVTDVAVELNKFAFMQLDNPEMKKREID
FCHGPLCGTGGLEAAVKEQQDGKCLLCGKESIGHYHHIVPRSRRGSNTIANIA
GLCPKCHELVHKDADTAESLTEMKTGLMKKYGGTSVLNQIIPKLVETLADLFP
GHFHVTNGWNTKEFREKHHLEKDHDVDAYCIACSHLKPEETLVETEPFEILQF
RKHNRAIIHHQTERTYKLDGVTVAKNRKKRMEQKTDSLEDWYVDMAKEHG
KTQADAMRSRLTVIKSTRYYNTPGRMMPGTVFLYEGKRYVMTGQITNGKYY
RAYGQEKRNFPAVKVRILTKNTGLVFVA;
(SEQ ID NO: 2; pEDIT071)
MRYVYVLDVDGKPLMPTCRFGKVRRMLKSGQAKAVDTLPFTIQLTYKPRTRI
LQPVTLGQDPGRTNIGMAAVRFDGKELGRFHCITRNKEIPKLMADRMAARKA
SRRGERLARKRLARKLHTTAKHLNGRILPGCSEPIAVKDIINTESRFNNRILTKC
KVCGKNTPLRRNVRELLLENIVRFLPLESELKETLKRTILEGQQGNINKLFRKL
KFNQKDWPGKNLTDIAKNKLPGRLPFCKEHFAENEKFTTIEKSTFRLTPTATQL
LRTHINLFRKLSGILPVTDVAVELNKFAFMQLDNPEMKKREIDFCHGPLCGTG
GLEAAVKEQQDGKCLLCGKESIGHYHHIVPRSRRGSNTIANIAGLCPKCHELV
HKDADTAESLTEMKTGLMKKYGGTSVLNQIIPKLVETLADLFPGHFHVTNGW
NTKEFREKHHLEKDHDVDAYCIACSHLKPEETLVETEPFEILQFRKHNRAIIHH
QTERTYKLDGVTVAKNRKKRMEQKTDSLEDWYVDMAKEHGKTQADAMRS
RLTVIKSTRYYNTPGRMMPGTVFLYEGKRYVMTGQITNGKYYRAYGQEKRN
FPAVKVRILTKNTGLVFVA;
(SEQ ID NO: 3; pEDIT416)
MRYVYVLDVDGKPLMPTCRFGKVRRMLKSGQAKAVDTLPFTIQLTYKPRTRI
LQPVTLGQDPGRTNIGMAAVRFDGKELGRFHCITRNKEIPKLMADRMAARKA
SRRGERLARKRLARKLHTTAKHLNGRILPGCSKPIAVKDIINTESRFNNRILTKC
KVCGKNTPLRRNVRELLLENIVRFLPLESELKETLKRTILEGQQGNINKLFRKL
KFNQKDWPGKNLTDIAKNKLPGRLPFCKEHFAENEKFTTIEKSTFRLTPTATQL
LRTHINLFRKLSGILPVTDVAVELNKFAFMQLDNPEMKKREIDFCHGPLCGTG
GLEAAVKEQQDGKCLLCGKESIGHYHHIVPRSRRGSNTIANIAGLCPKCHELV
HKDADTAESLTEMKTGLMKKYGGTSVLNQIIPKLVRTLAKLFPGHFHVTNGW
NTKEFREKHHLEKDHDVDAYCIACSHLKPEETLVETEPFEILQFRKHNRAIIHH
QTERTYKLDGVTVAKNRKKRMEQKTDSLEDWYVDMAKEHGKTQADAMRS
RLTVKKSRRYYNTPGRMMPGTVFLYEGKRYVMTGQITNGKYYRAYGQEKRN
FPAVKVRILTKNTGLVFVA;
(SEQ ID NO: 4 pEDIT417)
MRYVYVLDVDGKPLMPTCRFGKVRRMLKSGQAKAVDTLPFTIQLTYKPRTRI
LQPVTLGQDPGRTHIGMAAVRFDGKELGRFHCITRNKEIPKLMADRMKARKA
SRRGERLARKRLARKLHTTAKHLNGRILPGCSKPIAVKDIINTESRFNNRILTKC
KVCGKNTPLRRNVRELLLENIVRFLPLESELKETLKRTILEGQQGNINKLFRKL
KFNQKDWPGKNLTDIAKNKLPGRLPFCKEHFAENEKFTTIEKSTFRLTPTATQL
LRTHINLFRKLSGILPVTDVAVELNKFAFMQLDNPEMKKREIDFCHGPLCGTG
GLEAAVKEQQDGKCLLCGKESIGHYHHIVPRSRRGSNTIANIAGLCPKCHELV
HKDADTAESLTEMKTGLMKKYGGTSVLNQIIPKLVRTLAKLFPGHFHVTNGW
NTKEFREKHHLEKDHDVDAYCIACSHLKPEETLVETEPFEILQFRKHNRAIIHH
QTERTYKLDGVTVAKNRKKRMEQKTDSLEDWYVDMAKEHGKTQADAMRS
RLTVKKSTRYYNTPGRMMPGTVFLYEGKRYVMTGQITNGKYYRAYGQEKRN
FPAVKVRILTKNTGLVFVA;
(SEQ ID NO: 5; pEDIT418)
MRYVYVLDVDGKPLMPTCRFGKVRRMLKSGQAKAVDTLPFTIQLTYKPRTRI
LQPVTLGQDPGRTHIGMAAVRFDGKELGRFHCITRNKEIPKLMADRMAARKA
SRRGERLARKRLARKLHTTAKHLNGRILPGCSKPIAVKDIINTESRFNNRILTKC
KVCGKNTPLRRNVRELLLENIVRFLPLESELKETLKRTILEGQQGNINKLFRKL
KFNQKDWPGKNLTDIAKNKLPGRLPFCKEHFAENEKFTTIEKSTFRLTPTATQL
LRTHINLFRKLSGILPVTDVAVELNKFAFMQLDNPEMKKREIDFCHGPLCGTG
GLEAAVKEQQDGKCLLCGKESIGHYHHIVPRSRRGSNTIANIAGLCPKCHELV
HKDADTAESLTEMKTGLMKKYGGTSVLNQIIPKLVRTLAKLFPGHFHVTNGW
NTKEFREKHHLEKDHDVDAYCIACSHLKPEETLVETEPFEILQFRKHNRAIIHH
QTERTYKLDGVTVAKNRKKRMEQKTDSLEDWYVDMAKEHGKTQADAMRS
RLTVKKSRRYYNTPGRMMPGTVFLYEGKRYVMTGQITNGKYYRAYGQEKRN
FPAVKVRILTKNTGLVFVA;
(SEQ ID NO: 6; pEDIT0152, triple-mutant)
MGSPKKKRKVGSMRYVYVLDVDGKPLMPTCRFGKVRRMLKSGQAKAVDTL
PFTIQLTYKPRTRILQPVTLGQDPGRTNIGMAAVRFDGKELGRFHCITRNKEIPK
LMADRMAARKASRRGERLARKRLARKLHTTAKHLNGRILPGCSEPIAVKDIIN
TESRFNNRILTKCKVCGKNTPLRRNVRELLLENIVRFLPLESELKETLKRTILEG
QQGNINKLFRKLKFNQKDWPGKNLTDIAKNKLPGRLPFCKEHFAENEKFTTIE
KSTFRLTPTATQLLRTHINLFRKLSGILPVTDVAVELNKFAFMQLDNPEMKKR
EIDFCHGPLCGTGGLEAAVKEQQDGKCLLCGKESIGHYHHIVPRSRRGSNTIA
NIAGLCPKCHELVHKDADTAESLTEMKTGLMKKYGGTSVLNQIIPKLVRTLA
KLFPGHFHVTNGWNTKEFREKHHLEKDHDVDAYCIACSHLKPEETLVETEPFE
ILQFRKHNRAIIHHQTERTYKLDGVTVAKNRKKRMEQKTDSLEDWYVDMAK
EHGKTQADAMRSRLTVKKSTRYYNTPGRMMPGTVFLYEGKRYVMTGQITNG
KYYRAYGQEKRNFPAVKVRILTKNTGLVFVAGSKRPAATKKAGQAKKKKGS
YPYDVPDYAYPYDVPDYAYPYDVPDYA;
or
(SEQ ID NO: 7; pEDIT0420; 7-mutant)
MGSPKKKRKVGSMRYVYVLDVDGKPLMPTCRFGKVRRMLKSGQAKAVDTL
PFTIQLTYKPRTRILQPVTLGQDPGRTHIGMAAVRFDGKELGRFHCITRNKEIPK
LMADRMKARKASRRGERLARKRLARKLHTTAKHLNGRILPGCSKPIAVKDIIN
TESRFNNRILTKCKVCGKNTPLRRNVRELLLENIVRFLPLESELKETLKRTILEG
QQGNINKLFRKLKFNQKDWPGKNLTDIAKNKLPGRLPFCKEHFAENEKFTTIE
KSTFRLTPTATQLLRTHINLFRKLSGILPVTDVAVELNKFAFMQLDNPEMKKR
EIDFCHGPLCGTGGLEAAVKEQQDGKCLLCGKESIGHYHHIVPRSRRGSNTIA
NIAGLCPKCHELVHKDADTAESLTEMKTGLMKKYGGTSVLNQIIPKLVRTLA
KLFPGHFHVTNGWNTKEFREKHHLEKDHDVDAYCIACSHLKPEETLVETEPFE
ILQFRKHNRAIIHHQTERTYKLDGVTVAKNRKKRMEQKTDSLEDWYVDMAK
EHGKTQADAMRSRLTVKKSRRYYNTPGRMMPGTVFLYEGKRYVMTGQITNG
KYYRAYGQEKRNFPAVKVRILTKNTGLVFVAGSKRPAATKKAGQAKKKKGS
YPYDVPDYAYPYDVPDYAYPYDVPDYA.
(SEQ ID NO: 8; pEDIT071 + NLS)
MGSPKKKRKVGSMRYVYVLDVDGKPLMPTCRFGKVRRMLKSGQAKAVDTL
PFTIQLTYKPRTRILQPVTLGQDPGRTNIGMAAVRFDGKELGRFHCITRNKEIPK
LMADRMAARKASRRGERLARKRLARKLHTTAKHLNGRILPGCSEPIAVKDIIN
TESRFNNRILTKCKVCGKNTPLRRNVRELLLENIVRFLPLESELKETLKRTILEG
QQGNINKLFRKLKFNQKDWPGKNLTDIAKNKLPGRLPFCKEHFAENEKFTTIE
KSTFRLTPTATQLLRTHINLFRKLSGILPVTDVAVELNKFAFMQLDNPEMKKR
EIDFCHGPLCGTGGLEAAVKEQQDGKCLLCGKESIGHYHHIVPRSRRGSNTIA
NIAGLCPKCHELVHKDADTAESLTEMKTGLMKKYGGTSVLNQIIPKLVETLAD
LFPGHFHVTNGWNTKEFREKHHLEKDHDVDAYCIACSHLKPEETLVETEPFEI
LQFRKHNRAIIHHQTERTYKLDGVTVAKNRKKRMEQKTDSLEDWYVDMAKE
HGKTQADAMRSRLTVIKSTRYYNTPGRMMPGTVFLYEGKRYVMTGQITNGK
YYRAYGQEKRNFPAVKVRILTKNTGLVFVA

In some embodiments, the protein effector polypeptide comprises an amino acid sequence comprising a mutation at least one, at least two, at least three, at least four, at least five, at least six, at least seven, at least eight, or at least nine of positions 18, 34, 36, 37, 60, 66, 85, 97, 99, 100, 101, 104, 109, 112, 118, 136, 137, 155, 156, 157, 166, 168, 190, 204, 204, 205, 206, 208, 212, 214, 216, 218, 219, 229, 229, 230, 233, 233, 234, 234, 238, 239, 241, 242, 245, 246, 248, 252, 254, 255, 256, 257, 258, 279, 280, 291, 293, 298, 303, 304, 306, 307, 312, 319, 323, 328, 329, 331, 332, 338, 341, 353, 360, 368, 372, 376, 377, 378, 379, 380, 381, 382, 384, 385, 386, 388, 390, 391, 393, 405, 406, 408, 409, 410, 412, 413, 416, 417, 418, 419, 421, 421, 423, 425, 437, 439, 455, 462, 463, 478, 479, 533, 536, 564 and 565 of the amino acid sequence of SEQ ID NO: 2 (pEDIT071). Optionally, if the protein effector polypeptide comprises the mutation Q60I, N66H, I85R, I85K, E137R, P405K, E409K, E409R, or D413R, the protein effector polypeptide comprises a mutation at least one other position from the above and/or a mutation other than Q60I, N66H, I85R, I85K, E137R, P405K, E409K, E409R, or D413R. Without wishing to be bound by a theory, protein effector polypeptide comprises an amino acid sequence comprising a mutation at one or more of the above positions have higher or increased gene-editing efficiency than the protein effector polypeptide comprising the amino acid sequence of SEQ ID NO: 2. In some embodiments, the protein effector polypeptide comprising a mutation at one or more of the above listed positions has at least 12-fold, at least 3-fold, at least 4-fold, at least 5-fold, at least 10-fold, at least 15-fold, at least 20-fold, at least 25-fold, at least 30-fold, at least 35 fold, at least 40-fold, at least 45 fold, or at least 50-fold higher editing efficiency than the protein effector polypeptide comprising the amino acid sequence of SEQ ID NO: 2.

In some embodiments, the protein effector polypeptide comprises an amino acid sequence comprising at least one, at least two, at least three, at least four, at least five, at least six, at least seven, at least eight, or at least nine mutations selected from the group consisting of variant C18R, A34R, D36R, T37R, Q60I, N66H, I85X, 185X, M99X, A101X, A104R, E109X, A112K, R118K, S136X, E137R, I155X, L156X, T157X, T166X, L168K, L190K, G204X, N205X, I206K, K208R, K212R, K214R, N216R, K218R, D219X, A229X, K230R, L233X, P234X, P238X, F239X, K241R, E242X, A245X, E246X, E248R, T252X, E254X, K255R, S256X, T257X, F258X, S279R, G280R, L291X, K293R, Q298K, E303X, M304K, K306R, R307K, C312X, T319K, E323X, E328R, Q329R, D331R, G332R, G338R, S341R, R353K, A360X, K368R, L372R, D376R, A377R, D378R, T379R, A380R, E381R, S382R, T384R, E385R, M386R, T388R, L390X, M391X, K393R, P405K, K406R, V408R, E409X, T410R, A412R, D413R, P416R, G417R, H418K, F419R, V421X, N423R, W425K, E437R, D439X, E455R, E462R, P463R, H478X, H479X, I564X, and T565X of the amino acid sequence provided in SEQ ID NO: 2, wherein X is any amino acid. Optionally, if the protein effector polypeptide comprises the mutation Q60I, N66H, I85R, I85K, E137R, P405K, E409K, E409R, or D413R, the protein effector polypeptide comprises a mutation at least one other position from the above and/or a mutation other than Q60I, N66H, I85R, I85K, E137R, P405K, E409K, E409R, or D413R.

Some exemplary specific mutations of SEQ ID NO: 2 are shown in Table A.

TABLE A
Exemplary positions and mutations for SEQ ID NO: 2.

C18R, A34R, D36R, T37R, Q60I, N66H, I85X, I85X, M99X, A101X, A104R, E109X, A112K,
R118K, S136X, E137R, I155X, L156X, T157X, T166X, L168K, L190K, G204X, N205X, I206K,
K208R, K212R, K214R, N216R, K218R, D219X, A229X, K230R, L233X, P234X, P238X, F239X,
K241R, E242X, A245X, E246X, E248R, T252X, E254X, K255R, S256X, T257X, F258X, S279R,
G280R, L291X, K293R, Q298K, E303X, M304K, K306R, R307K, C312X, T319K, E323X, E328R,
Q329R, D331R, G332R, G338R, S341R, R353K, A360X, K368R, L372R, D376R, A377R, D378R,
T379R, A380R, E381R, S382R, T384R, E385R, M386R, T388R, L390X, M391X, K393R, P405K,
K406R, V408R, E409X, T410R, A412R, D413R, P416R, G417R, H418K, F419R, V421X, N423R,
W425K, E437R, D439X, E455R, E462R, P463R, H478N, H479N, I564K, I564H, I564N, I564Q,
I564C, I564M, I564T, I564D, T565R, T565K, T565H, T565C, T565D, T565G, H478C, H478P,
H479C, and H479M

Some exemplary specific mutations of SEQ ID NO: 8 or SEQ ID NO: 7 are shown in Table A2.

TABLE A2
Exemplary positions and mutations for SEQ ID NO: 8 or SEQ ID NO: 7.

C30R, A46R, D46R, T49R, Q72I, N78H, I97R, I97K, D109R, D109K, M111R, M111K, A112K,
A113R, A113K, A116R, E121R, E121K, A124K, R130K, S148R, S148K, E149R, E149K, I167R,
I167K, L168R, L168K, T169R, T169K, T178R, T178K, L180K, L1202K, G216R, G216K, N217R,
N217K, I218K, K220R, K224R, K2226R, N228R, K230R, D231R, D231K, A241R, A241K,
K242R, L245R, L245K, P246R, P246K, P250R, P250K, F251R, F251K, K253R, E254R, E2454K,
A257R, A257K, E258R, E2458K, E260R, T264R, T264K, E266R, E266K, K267R, S268R, S268K,
T2569R, T2569K, F270R, F270K, S291R, G292R, L303R, L303K, K305R, Q310K, E315R, E315K,
M316K, K318R, R319K, C324R, C324K, T331K, E335R, E335K, E340R, Q341R, D343R, G344R,
G350R, S353R, R365K, A372R, A372K, K380R, L384R, D388R, A389R, D390R, T391R, A392R,
E393R, S394R, T396R, E397R, M398R, T400R, L402R, L402K, M4031R, M403K, K405R, P417K,
K419R, V420R, E421K, E421R, T422R, A424R, D425R, D425K, P428R, G429R, H430K, F431R,
V433R, V433K, N435R, W437K, E449R, D451R, D451K, E4567R, E474R, P475R, I545K, T548R,
H490N, H491N, I576K, I576H, I576N, I576Q, I576C, I576M, I576T, I5676D, T577R, T577K,
T577H, T577C, T577D, T577G, H490C, H490P, H491C, and H491M

In some embodiments, the protein effector polypeptide comprises an amino acid sequence comprising at least one, at least two, at least three, at least four, at least five, at least six, at least seven, at least eight, or at least nine mutations selected from the group consisting of variant C18R, A34R, D36R, T37R, Q60I, N66H, I85X, 185X, M99X, A101X, A104R, E109X, A112K, R118K, S136X, E137R, I155X, L156X, T157X, T166X, L168K, L190K, G204X, N205X, I206K, K208R, K212R, K214R, N216R, K218R, D219X, A229X, K230R, L233X, P234X, P238X, F239X, K241R, E242X, A245X, E246X, E248R, T252X, E254X, K255R, S256X, T257X, F258X, S279R, G280R, L291X, K293R, Q298K, E303X, M304K, K306R, R307K, C312X, T319K, E323X, E328R, Q329R, D331R, G332R, G338R, S341R, R353K, A360X, K368R, L372R, D376R, A377R, D378R, T379R, A380R, E381R, S382R, T384R, E385R, M386R, T388R, L390X, M391X, K393R, P405K, K406R, V408R, E409X, T410R, A412R, D413R, P416R, G417R, H418K, F419R, V421X, N423R, W425K, E437R, D439X, E455R, E462R, P463R, H478N, H479N, I564K, I564H, I564N, I564Q, I564C, I564M, I564T, I564D, T565R, T565K, T565H, T565C, T565D, T565G, H478C, H478P, H479C, and H479M of the amino acid sequence provided in SEQ ID NO: 1, wherein X is any amino acid. Optionally, if the protein effector polypeptide comprises the mutation Q60I, N66H, I85R, I85K, E137R, P405K, E409K, E409R, or D413R, the protein effector polypeptide comprises a mutation at least one other position from the above and/or a mutation other than Q60I, N66H, I85R, I85K, E137R, P405K, E409K, E409R, or D413R.

In some embodiments, the protein effector polypeptide comprises an amino acid sequence comprising a mutation at least one, at least two, at least three, at least four, at least five, at least six, or at least seven of positions 409, 413, 533, 66, 100, 137 and 536 of the amino acid sequence of SEQ ID NO: 2. Optionally, if the protein effector polypeptide comprises the mutation N66H, I85R, I85K, E137R, P405K, E409K, E409R, or D413R, the protein effector polypeptide comprises a mutation at least one other position from the above and/or a mutation other than N66H, E137K, E409K, E409R, or D413R, the protein effector polypeptide comprises a mutation at least one (e.g., at least two, at least three, at least four, at least five or more) other position (e.g., at position 18, 34, 36, 37, 60, 85, 97, 99, 100, 101, 104, 109, 112, 118, 136, 155, 156, 157, 166, 168, 190, 204, 204, 205, 206, 208, 212, 214, 216, 218, 219, 229, 229, 230, 233, 233, 234, 234, 238, 239, 241, 242, 245, 246, 248, 252, 254, 255, 256, 257, 258, 279, 280, 291, 293, 298, 303, 304, 306, 307, 312, 319, 323, 328, 329, 331, 332, 338, 341, 353, 360, 368, 372, 376, 377, 378, 379, 380, 381, 382, 384, 385, 386, 388, 390, 391, 393, 405, 406, 408, 410, 412, 416, 417, 418, 419, 421, 421, 423, 425, 437, 439, 455, 462, 463, 533, or 536) and/or a mutation other than N66H, E137K, E409K, E409R, or D413R.

In some embodiments, the protein effector polypeptide comprises an amino acid sequence comprising a mutation at positions 409, 413, and 533 in the amino acid sequence of SEQ ID NO: 2. For example, the protein effector polypeptide comprises an amino acid sequence comprising the mutations E409K, D413K, and I533R in the amino acid sequence of SEQ ID NO: 2. Without wishing to be bound by a theory, protein effector polypeptide comprises an amino acid sequence comprising a mutation at positions 409, 413, and 533 in the amino acid sequence of SEQ ID NO: 2 have higher or increased gene-editing efficiency than the protein effector polypeptide comprising the amino acid sequence of SEQ ID NO: 2. In some embodiments, the protein effector polypeptide comprising mutations at least at positions 409, 413, and 533 has at least 12-fold, at least 3-fold, at least 4-fold, at least 5-fold, at least 10-fold, at least 15-fold, at least 20-fold, at least 25-fold, at least 30-fold, at least 35 fold, at least 40-fold, at least 45 fold, or at least 50-fold higher editing efficiency than the protein effector polypeptide comprising the amino acid sequence of SEQ ID NO: 2.

In some embodiments, the protein effector polypeptide comprises an amino acid sequence comprising a mutation at positions 421, 425, and 545 in the amino acid sequence of SEQ ID NO: 8. For example, the protein effector polypeptide comprises an amino acid sequence comprising the mutations E421K, D425K, and 1545R in the amino acid sequence of SEQ ID NO: 8. Without wishing to be bound by a theory, protein effector polypeptide comprises an amino acid sequence comprising a mutation at positions 421, 425, and 545 in the amino acid sequence of SEQ ID NO: 8 have higher or increased gene-editing efficiency than the protein effector polypeptide comprising the amino acid sequence of SEQ ID NO: 8. In some embodiments, the protein effector polypeptide comprising mutations at least at positions 421, 425, and 545 has at least 12-fold, at least 3-fold, at least 4-fold, at least 5-fold, at least 10-fold, at least 15-fold, at least 20-fold, at least 25-fold, at least 30-fold, at least 35 fold, at least 40-fold, at least 45 fold, or at least 50-fold higher editing efficiency than the protein effector polypeptide comprising the amino acid sequence of SEQ ID NO: 8.

### Triple+Modifications

In some embodiments, the protein effector polypeptide comprises an amino acid sequence comprising a mutation at positions 409, 413, and 533, and a mutation at least one (e.g., two, three or four) of position 66, 100, 137 536 of SEQ ID NO: 2. For example, the protein effector polypeptide comprises an amino acid sequence comprising the mutations E409K, D413K and I533R, and at least one (e.g., two, three, or four) of mutations N66H, A100K, E137K and T536R in the amino acid sequence of SEQ ID NO: 2.

In some embodiments, the protein effector polypeptide comprises an amino acid sequence comprising a mutation at positions 409, 413, 533, and 66 in the amino acid sequence of SEQ ID NO: 2. For example, the protein effector polypeptide comprises an amino acid sequence comprising the mutations E409K, D413K, I533R, and N66H in the amino acid sequence of SEQ ID NO: 2.

In some embodiments, the protein effector polypeptide comprises an amino acid sequence comprising a mutation at positions 409, 413, 533, and 100 in the amino acid sequence of SEQ ID NO: 2. For example, the protein effector polypeptide comprises an amino acid sequence comprising the mutations E409K, D413K, I533R, and A100K in the amino acid sequence of SEQ ID NO: 2.

In some embodiments, the protein effector polypeptide comprises an amino acid sequence comprising a mutation at position 409, 413, 533, and 137 in the amino acid sequence of SEQ ID NO: 2. For example, the protein effector polypeptide comprises an amino acid sequence comprising the mutations E409K, D413K, I533R, and E137K in the amino acid sequence of SEQ ID NO: 2.

In some embodiments, the protein effector polypeptide comprises an amino acid sequence comprising 409, 413, 533, and 536 in the amino acid sequence of SEQ ID NO: 2. For example, the protein effector polypeptide comprises an amino acid sequence comprising the mutations E409K, D413K, I533R, and T536R in the amino acid sequence of SEQ ID NO: 2.

In some embodiments, the protein effector polypeptide comprises an amino acid sequence comprising 409, 413, 533, 66, and 100 in the amino acid sequence of SEQ ID NO: 2. For example, the protein effector polypeptide comprises an amino acid sequence comprising the mutations E409K, D413K, I533R, N66H, and A100K in the amino acid sequence of SEQ ID NO: 2.

In some embodiments, the protein effector polypeptide comprises an amino acid sequence comprising 409, 413, 533, 66, and 137 in the amino acid sequence of SEQ ID NO: 2. For example, the protein effector polypeptide comprises an amino acid sequence comprising the mutations E409K, D413K, I533R, N66H, and E137K in the amino acid sequence of SEQ ID NO: 2

In some embodiments, the protein effector polypeptide comprises an amino acid sequence comprising 409, 413, 533, 66, and 536 in the amino acid sequence of SEQ ID NO: 2. For example, the protein effector polypeptide comprises an amino acid sequence comprising the mutations E409K, D413K, I533R, N66H, and T536R in the amino acid sequence of SEQ ID NO: 2.

In some embodiments, the protein effector polypeptide comprises an amino acid sequence comprising 409, 413, 533, 100, and 137 in the amino acid sequence of SEQ ID NO: 2. For example, the protein effector polypeptide comprises an amino acid sequence comprising the mutations E409K, D413K, I533R, A100K, and E137 in the amino acid sequence of SEQ ID NO: 2.

In some embodiments, the protein effector polypeptide comprises an amino acid sequence comprising 409, 413, 533, 100, and 536 in the amino acid sequence of SEQ ID NO: 2. For example, the protein effector polypeptide comprises an amino acid sequence comprising the mutations E409K, D413K, I533R, A100K, and T536R in the amino acid sequence of SEQ ID NO: 2.

In some embodiments, the protein effector polypeptide comprises an amino acid sequence comprising 409, 413, 533, 137 and 536 in the amino acid sequence of SEQ ID NO: 2. For example, the protein effector polypeptide comprises an amino acid sequence comprising the mutations E409K, D413K, I533R, E137K and T536R in the amino acid sequence of SEQ ID NO: 2.

In some embodiments, the protein effector polypeptide comprises an amino acid sequence comprising 409, 413, 533, 66, 100, and 137 in the amino acid sequence of SEQ ID NO: 2. For example, the protein effector polypeptide comprises an amino acid sequence comprising the mutations E409K, D413K, I533R, N66H, A100K, and E137K in the amino acid sequence of SEQ ID NO: 2.

In some embodiments, the protein effector polypeptide comprises an amino acid sequence comprising 409, 413, 533, 66, 100, and 536 in the amino acid sequence of SEQ ID NO: 2. For example, the protein effector polypeptide comprises an amino acid sequence comprising the mutations E409K, D413K, I533R, N66H, A100K, and T536R in the amino acid sequence of SEQ ID NO: 2.

In some embodiments, the protein effector polypeptide comprises an amino acid sequence comprising 409, 413, 533, 66, 137 and 536 in the amino acid sequence of SEQ ID NO: 2. For example, the protein effector polypeptide comprises an amino acid sequence comprising the mutations E409K, D413K, I533R, N66H, E137K and T536R in the amino acid sequence of SEQ ID NO: 2.

In some embodiments, the protein effector polypeptide comprises an amino acid sequence comprising 409, 413, 533, 100, 137 and 536 in the amino acid sequence of SEQ ID NO: 2. For example, the protein effector polypeptide comprises an amino acid sequence comprising the mutations E409K, D413K, I533R, A100K, E137K and T536R in the amino acid sequence of SEQ ID NO: 2.

In some embodiments, the protein effector polypeptide comprises an amino acid sequence comprising 409, 413, 533, 66, 100, 137 and 536 in the amino acid sequence of SEQ ID NO: 2. For example, the protein effector polypeptide comprises an amino acid sequence comprising the mutations E409K, D413K, I533R, N66H, A100K, E137K and T536R in the amino acid sequence of SEQ ID NO: 2.

In some embodiments, the protein effector polypeptide comprises an amino acid sequence comprising a mutation at positions 421, 423, and 545, and a mutation at least one (e.g., two, three or four) of position 78, 112, 149 548 of SEQ ID NO: 8. For example, the protein effector polypeptide comprises an amino acid sequence comprising the mutations E421K, D423K and I545R, and at least one (e.g., two, three, or four) of mutations N78H, A112K, E149K and T548R in the amino acid sequence of SEQ ID NO: 8.

In some embodiments, the protein effector polypeptide comprises an amino acid sequence comprising a mutation at positions 421, 423, 545, and 78 in the amino acid sequence of SEQ ID NO: 8. For example, the protein effector polypeptide comprises an amino acid sequence comprising the mutations E421K, D423K, I545R, and N78H in the amino acid sequence of SEQ ID NO: 8.

In some embodiments, the protein effector polypeptide comprises an amino acid sequence comprising a mutation at positions 421, 423, 545, and 112 in the amino acid sequence of SEQ ID NO: 8. For example, the protein effector polypeptide comprises an amino acid sequence comprising the mutations E421K, D423K, I545R, and A112K in the amino acid sequence of SEQ ID NO: 8.

In some embodiments, the protein effector polypeptide comprises an amino acid sequence comprising a mutation at position 421, 423, 545, and 149 in the amino acid sequence of SEQ ID NO: 8. For example, the protein effector polypeptide comprises an amino acid sequence comprising the mutations E421K, D423K, I545R, and E149K in the amino acid sequence of SEQ ID NO: 8.

In some embodiments, the protein effector polypeptide comprises an amino acid sequence comprising 421, 423, 545, and 548 in the amino acid sequence of SEQ ID NO: 8. For example, the protein effector polypeptide comprises an amino acid sequence comprising the mutations E421K, D423K, I545R, and T548R in the amino acid sequence of SEQ ID NO: 8.

In some embodiments, the protein effector polypeptide comprises an amino acid sequence comprising 421, 423, 545, 78, and 112 in the amino acid sequence of SEQ ID NO: 8. For example, the protein effector polypeptide comprises an amino acid sequence comprising the mutations E421K, D423K, I545R, N78H, and A112K in the amino acid sequence of SEQ ID NO: 8.

In some embodiments, the protein effector polypeptide comprises an amino acid sequence comprising 421, 423, 545, 78, and 149 in the amino acid sequence of SEQ ID NO: 8. For example, the protein effector polypeptide comprises an amino acid sequence comprising the mutations E421K, D423K, I545R, N78H, and E149K in the amino acid sequence of SEQ ID NO: 8

In some embodiments, the protein effector polypeptide comprises an amino acid sequence comprising 421, 423, 545, 78, and 548 in the amino acid sequence of SEQ ID NO: 8. For example, the protein effector polypeptide comprises an amino acid sequence comprising the mutations E421K, D423K, I545R, N78H, and T548R in the amino acid sequence of SEQ ID NO: 8.

In some embodiments, the protein effector polypeptide comprises an amino acid sequence comprising 421, 423, 545, 112, and 149 in the amino acid sequence of SEQ ID NO: 8. For example, the protein effector polypeptide comprises an amino acid sequence comprising the mutations E421K, D423K, I545R, A112K, and E149 in the amino acid sequence of SEQ ID NO: 8.

In some embodiments, the protein effector polypeptide comprises an amino acid sequence comprising 421, 423, 545, 112, and 548 in the amino acid sequence of SEQ ID NO: 8. For example, the protein effector polypeptide comprises an amino acid sequence comprising the mutations E421K, D423K, I545R, A112K, and T548R in the amino acid sequence of SEQ ID NO: 8.

In some embodiments, the protein effector polypeptide comprises an amino acid sequence comprising 421, 423, 545, 149 and 548 in the amino acid sequence of SEQ ID NO: 8. For example, the protein effector polypeptide comprises an amino acid sequence comprising the mutations E421K, D423K, I545R, E149K and T548R in the amino acid sequence of SEQ ID NO: 8.

In some embodiments, the protein effector polypeptide comprises an amino acid sequence comprising 421, 423, 545, 78, 112, and 149 in the amino acid sequence of SEQ ID NO: 8. For example, the protein effector polypeptide comprises an amino acid sequence comprising the mutations E421K, D423K, I545R, N78H, A112K, and E149K in the amino acid sequence of SEQ ID NO: 8.

In some embodiments, the protein effector polypeptide comprises an amino acid sequence comprising 421, 423, 545, 78, 112, and 548 in the amino acid sequence of SEQ ID NO: 8. For example, the protein effector polypeptide comprises an amino acid sequence comprising the mutations E421K, D423K, I545R, N78H, A112K, and T548R in the amino acid sequence of SEQ ID NO: 8.

In some embodiments, the protein effector polypeptide comprises an amino acid sequence comprising 421, 423, 545, 78, 149 and 548 in the amino acid sequence of SEQ ID NO: 8. For example, the protein effector polypeptide comprises an amino acid sequence comprising the mutations E421K, D423K, I545R, N78H, E149K and T548R in the amino acid sequence of SEQ ID NO: 8.

In some embodiments, the protein effector polypeptide comprises an amino acid sequence comprising 421, 423, 545, 112, 149 and 548 in the amino acid sequence of SEQ ID NO: 8. For example, the protein effector polypeptide comprises an amino acid sequence comprising the mutations E421K, D423K, I545R, A112K, E149K and T548R in the amino acid sequence of SEQ ID NO: 8.

In some embodiments, the protein effector polypeptide comprises an amino acid sequence comprising 421, 423, 545, 78, 112, 149 and 548 in the amino acid sequence of SEQ ID NO: 8. For example, the protein effector polypeptide comprises an amino acid sequence comprising the mutations E421K, D423K, I545R, N78H, A112K, E149K and T548R in the amino acid sequence of SEQ ID NO: 8.

### Stacking of Mutations

In some embodiments, the protein effector polypeptide comprises an amino acid sequence of SEQ ID NO: 2 and comprising a mutation at positions 409, 413, and 533, and at least one other mutation at a position listed in Table A. For example, the protein effector polypeptide comprises an amino acid sequence of SEQ ID NO: 2 and comprising the mutations E409K, D413K and I533R, and at least one other mutation listed in Table A.

In some embodiments, the protein effector polypeptide comprises an amino acid sequence of SEQ ID NO: 2 and comprising a mutation at positions 409, 413, 533 and 66, and at least one other mutation at a position listed in Table A. For example, the protein effector polypeptide comprises an amino acid sequence of SEQ ID NO: 2 and comprising the mutations E409K, D413K, I533R, and N66H, and at least one other mutation listed in Table A.

In some embodiments, the protein effector polypeptide comprises an amino acid sequence of SEQ ID NO: 2 and comprising a mutation at positions 409, 413, 533, and 100, and at least one other mutation at a position listed in Table A. For example, the protein effector polypeptide comprises an amino acid sequence of SEQ ID NO: 2 and comprising the mutations E409K, D413K, I533R, and A100K, and at least one other mutation listed in Table A.

In some embodiments, the protein effector polypeptide comprises an amino acid sequence of SEQ ID NO: 2 and comprising a mutation at positions 409, 413, 533, and 137, and at least one other mutation at a position listed in Table A. For example, the protein effector polypeptide comprises an amino acid sequence of SEQ ID NO: 2 and comprising the mutations E409K, D413K, I533R, and E137K, and at least one other mutation listed in Table A.

In some embodiments, the protein effector polypeptide comprises an amino acid sequence of SEQ ID NO: 2 and comprising 409, 413, 533, and 536, and at least one other mutation listed in Table A. For example, the protein effector polypeptide comprises an amino acid sequence of SEQ ID NO: 2 and comprising the mutations E409K, D413K, I533R, and T536R, and at least one other mutation listed in Table A.

In some embodiments, the protein effector polypeptide comprises an amino acid sequence of SEQ ID NO: 2 and comprising 409, 413, 533, 66, and 100, and at least one other mutation listed in Table A. For example, the protein effector polypeptide comprises an amino acid sequence of SEQ ID NO: 2 and comprising the mutations E409K, D413K, I533R, N66H, and A100K, and at least one other mutation listed in Table A.

In some embodiments, the protein effector polypeptide comprises an amino acid sequence of SEQ ID NO: 2 and comprising 409, 413, 533, 66, and 137, and at least one other mutation listed in Table A. For example, the protein effector polypeptide comprises an amino acid sequence of SEQ ID NO: 2 and comprising the mutations E409K, D413K, I533R, N66H, and E137K, and at least one other mutation listed in Table A.

In some embodiments, the protein effector polypeptide comprises an amino acid sequence of SEQ ID NO: 2 and comprising 409, 413, 533, 66, and 536, and at least one other mutation listed in Table A. For example, the protein effector polypeptide comprises an amino acid sequence of SEQ ID NO: 2 and comprising the mutations E409K, D413K, I533R, N66H, and T536R, and at least one other mutation listed in Table A.

In some embodiments, the protein effector polypeptide comprises an amino acid sequence of SEQ ID NO: 2 and comprising 409, 413, 533, 100, and 137, and at least one other mutation listed in Table A. For example, the protein effector polypeptide comprises an amino acid sequence of SEQ ID NO: 2 and comprising the mutations E409K, D413K, I533R, A100K, and E137, and at least one other mutation listed in Table A.

In some embodiments, the protein effector polypeptide comprises an amino acid sequence of SEQ ID NO: 2 and comprising 409, 413, 533, 100, and 536, and at least one other mutation listed in Table A. For example, the protein effector polypeptide comprises an amino acid sequence of SEQ ID NO: 2 and comprising the mutations E409K, D413K, I533R, A100K, and T536R, and at least one other mutation listed in Table A.

In some embodiments, the protein effector polypeptide comprises an amino acid sequence of SEQ ID NO: 2 and comprising 409, 413, 533, 137 and 536, and at least one other mutation listed in Table A. For example, the protein effector polypeptide comprises an amino acid sequence of SEQ ID NO: 2 and comprising the mutations E409K, D413K, I533R, E137K and T536R, and at least one other mutation listed in Table A.

In some embodiments, the protein effector polypeptide comprises an amino acid sequence of SEQ ID NO: 2 and comprising 409, 413, 533, 66, 100, and 137, and at least one other mutation listed in Table A. For example, the protein effector polypeptide comprises an amino acid sequence of SEQ ID NO: 2 and comprising the mutations E409K, D413K, I533R, N66H, A100K, and E137K, and at least one other mutation listed in Table A.

In some embodiments, the protein effector polypeptide comprises an amino acid sequence of SEQ ID NO: 2 and comprising 409, 413, 533, 66, 100, and 536, and at least one other mutation listed in Table A. For example, the protein effector polypeptide comprises an amino acid sequence of SEQ ID NO: 2 and comprising the mutations E409K, D413K, I533R, N66H, A100K, and T536R, and at least one other mutation listed in Table A.

In some embodiments, the protein effector polypeptide comprises an amino acid sequence of SEQ ID NO: 2 and comprising 409, 413, 533, 66, 137 and 536, and at least one other mutation listed in Table A. For example, the protein effector polypeptide comprises an amino acid sequence of SEQ ID NO: 2 and comprising the mutations E409K, D413K, I533R, N66H, E137K and T536R, and at least one other mutation listed in Table A.

In some embodiments, the protein effector polypeptide comprises an amino acid sequence of SEQ ID NO: 2 and comprising 409, 413, 533, 100, 137 and 536, and at least one other mutation listed in Table A. For example, the protein effector polypeptide comprises an amino acid sequence of SEQ ID NO: 2 and comprising the mutations E409K, D413K, I533R, A100K, E137K and T536R, and at least one other mutation listed in Table A.

In some embodiments, the protein effector polypeptide comprises an amino acid sequence of SEQ ID NO: 2 and comprising 409, 413, 533, 66, 100, 137 and 536, and at least one other mutation listed in Table A. For example, the protein effector polypeptide comprises an amino acid sequence of SEQ ID NO: 2 and comprising the mutations E409K, D413K, I533R, N66H, A100K, E137K and T536R, and at least one other mutation listed in Table A.

In some embodiments, the protein effector polypeptide comprises an amino acid sequence of SEQ ID NO: 8 and comprising a mutation at positions 421, 423, and 545, and at least one other mutation at a position listed in Table A2. For example, the protein effector polypeptide comprises an amino acid sequence of SEQ ID NO: 8 and comprising the mutations E421K, D423K and I545R, and at least one other mutation listed in Table A2.

In some embodiments, the protein effector polypeptide comprises an amino acid sequence of SEQ ID NO: 8 and comprising a mutation at positions 421, 423, 545 and 78, and at least one other mutation at a position listed in Table A2. For example, the protein effector polypeptide comprises an amino acid sequence of SEQ ID NO: 8 and comprising the mutations E421K, D423K, I545R, and N78H, and at least one other mutation listed in Table A2.

In some embodiments, the protein effector polypeptide comprises an amino acid sequence of SEQ ID NO: 8 and comprising a mutation at positions 421, 423, 545, and 112, and at least one other mutation at a position listed in Table A2. For example, the protein effector polypeptide comprises an amino acid sequence of SEQ ID NO: 8 and comprising the mutations E421K, D423K, I545R, and A112K, and at least one other mutation listed in Table A2.

In some embodiments, the protein effector polypeptide comprises an amino acid sequence of SEQ ID NO: 8 and comprising a mutation at positions 421, 423, 545, and 149, and at least one other mutation at a position listed in Table A2. For example, the protein effector polypeptide comprises an amino acid sequence of SEQ ID NO: 8 and comprising the mutations E421K, D423K, I545R, and E149K, and at least one other mutation listed in Table A2.

In some embodiments, the protein effector polypeptide comprises an amino acid sequence of SEQ ID NO: 8 and comprising 421, 423, 545, and 548, and at least one other mutation listed in Table A2. For example, the protein effector polypeptide comprises an amino acid sequence of SEQ ID NO: 8 and comprising the mutations E421K, D423K, I545R, and T548R, and at least one other mutation listed in Table A2.

In some embodiments, the protein effector polypeptide comprises an amino acid sequence of SEQ ID NO: 8 and comprising 421, 423, 545, 78, and 112, and at least one other mutation listed in Table A2. For example, the protein effector polypeptide comprises an amino acid sequence of SEQ ID NO: 8 and comprising the mutations E421K, D423K, I545R, N78H, and A112K, and at least one other mutation listed in Table A2.

In some embodiments, the protein effector polypeptide comprises an amino acid sequence of SEQ ID NO: 8 and comprising 421, 423, 545, 78, and 149, and at least one other mutation listed in Table A2. For example, the protein effector polypeptide comprises an amino acid sequence of SEQ ID NO: 8 and comprising the mutations E421K, D423K, I545R, N78H, and E149K, and at least one other mutation listed in Table A2.

In some embodiments, the protein effector polypeptide comprises an amino acid sequence of SEQ ID NO: 8 and comprising 421, 423, 545, 78, and 548, and at least one other mutation listed in Table A2. For example, the protein effector polypeptide comprises an amino acid sequence of SEQ ID NO: 8 and comprising the mutations E421K, D423K, I545R, N78H, and T548R, and at least one other mutation listed in Table A2.

In some embodiments, the protein effector polypeptide comprises an amino acid sequence of SEQ ID NO: 8 and comprising 421, 423, 545, 112, and 149, and at least one other mutation listed in Table A2. For example, the protein effector polypeptide comprises an amino acid sequence of SEQ ID NO: 8 and comprising the mutations E421K, D423K, I545R, A112K, and E149, and at least one other mutation listed in Table A2.

In some embodiments, the protein effector polypeptide comprises an amino acid sequence of SEQ ID NO: 8 and comprising 421, 423, 545, 112, and 548, and at least one other mutation listed in Table A2. For example, the protein effector polypeptide comprises an amino acid sequence of SEQ ID NO: 8 and comprising the mutations E421K, D423K, I545R, A112K, and T548R, and at least one other mutation listed in Table A2.

In some embodiments, the protein effector polypeptide comprises an amino acid sequence of SEQ ID NO: 8 and comprising 421, 423, 545, 149 and 548, and at least one other mutation listed in Table A2. For example, the protein effector polypeptide comprises an amino acid sequence of SEQ ID NO: 8 and comprising the mutations E421K, D423K, I545R, E149K and T548R, and at least one other mutation listed in Table A2.

In some embodiments, the protein effector polypeptide comprises an amino acid sequence of SEQ ID NO: 8 and comprising 421, 423, 545, 78, 112, and 149, and at least one other mutation listed in Table A2. For example, the protein effector polypeptide comprises an amino acid sequence of SEQ ID NO: 8 and comprising the mutations E421K, D423K, I545R, N78H, A112K, and E149K, and at least one other mutation listed in Table A2.

In some embodiments, the protein effector polypeptide comprises an amino acid sequence of SEQ ID NO: 8 and comprising 421, 423, 545, 78, 112, and 548, and at least one other mutation listed in Table A2. For example, the protein effector polypeptide comprises an amino acid sequence of SEQ ID NO: 8 and comprising the mutations E421K, D423K, I545R, N78H, A112K, and T548R, and at least one other mutation listed in Table A2.

In some embodiments, the protein effector polypeptide comprises an amino acid sequence of SEQ ID NO: 8 and comprising 421, 423, 545, 78, 149 and 548, and at least one other mutation listed in Table A2. For example, the protein effector polypeptide comprises an amino acid sequence of SEQ ID NO: 8 and comprising the mutations E421K, D423K, I545R, N78H, E149K and T548R, and at least one other mutation listed in Table A2.

In some embodiments, the protein effector polypeptide comprises an amino acid sequence of SEQ ID NO: 8 and comprising 421, 423, 545, 112, 149 and 548, and at least one other mutation listed in Table A2. For example, the protein effector polypeptide comprises an amino acid sequence of SEQ ID NO: 8 and comprising the mutations E421K, D423K, I545R, A112K, E149K and T548R, and at least one other mutation listed in Table A2.

In some embodiments, the protein effector polypeptide comprises an amino acid sequence of SEQ ID NO: 8 and comprising 421, 423, 545, 78, 112, 149 and 548, and at least one other mutation listed in Table A2. For example, the protein effector polypeptide comprises an amino acid sequence of SEQ ID NO: 8 and comprising the mutations E421K, D423K, I545R, N78H, A112K, E149K and T548R, and at least one other mutation listed in Table A2.

### Mutations for TAM Variants

In some embodiments, the protein effector polypeptide comprises an amino acid sequence comprising a mutation at one, two, three or four of positions 478, 479, 564 and 565 of the amino acid of SEQ ID NO: 2. For example, the protein effector polypeptide comprises an amino acid sequence comprising a mutation at least at positions 478 and 479, at positions 478 and 565, at positions 478 and 565, at positions 479 and 564, at positions 479 and 565, at positions 565 and 565, at positions 478, 479 and 564, at positions 478, 479 and 565, at positions 478, 564 and 565, at positions 479, 564 and 565, or at positions 478, 479, 564 and 565 of SEQ ID NO: 2.

It is noted that protein effector polypeptide comprising mutations at one, two, three or four of positions 478, 479, 564 and 565 of the amino acid of SEQ ID NO: 2 exhibit increased activity on a target sequence comprising a non-canonical TMA sequence. They are particularly effective on target sequences comprising the TAM sequence RTR, where R is purine (e.g., G or A). In some embodiments, the protein effector polypeptide comprising mutations at one, two, three or four of positions 478, 479, 564 and 565 of the amino acid of SEQ ID NO: 2 exhibit increased activity on a target sequence comprising the TAM sequence ATR. Particularly, the protein effector polypeptide comprising mutations at one, two, three or four of positions 478, 479, 564 and 565 of the amino acid of SEQ ID NO: 2 exhibit increased activity on a target sequence comprising the TAM sequence ATR in human cells.

In some embodiments, the t protein effector polypeptide comprising mutations at one, two, three or four of positions 478, 479, 564 and 565 of the amino acid of SEQ ID NO: 2 exhibits increased activity on a target sequence comprising a TAM sequence selected from the group consisting of RTAAA, ATRAA, ATRMM, ATRA, ATRRA, ATARA, ATRRAC, ATAAAC, ATARM, ATRAM, ATRRM, ATRMA, ATANA, ATRR, ATR, ATGAC, ATAA, ATAR, ATGMC, ATGAA, and ATRAR (where R is purine (e.g., A or G) and M is A) at its 3′ end as compared to SEQ ID NO: 2.

In some embodiments, the protein effector polypeptide comprises an amino acid sequence comprising a mutation at position 478 in the amino acid sequence of SEQ ID NO: 2. For example, the protein effector polypeptide comprises an amino acid sequence comprising a H478N, H478C or H479P mutation in the amino acid sequence of SEQ ID NO: 2. In some preferred embodiments, the protein effector polypeptide comprises an amino acid sequence comprising a H478N mutation in the amino acid sequence of SEQ ID NO: 2.

In some embodiments, the protein effector polypeptide comprises an amino acid sequence comprising a mutation at position 479 in the amino acid sequence of SEQ ID NO: 2. For example, the protein effector polypeptide comprises an amino acid sequence comprising a H479N, H479C or H479M mutation in the amino acid sequence of SEQ ID NO: 2. In some preferred embodiments, the protein effector polypeptide comprises an amino acid sequence comprising a H479N mutation in the amino acid sequence of SEQ ID NO: 2.

In some embodiments, the protein effector polypeptide comprises an amino acid sequence comprising a mutation at position 564 in the amino acid sequence of SEQ ID NO: 2. For example, the protein effector polypeptide comprises an amino acid sequence comprising a I564R, I564K, I564H, I564N, I564Q, I564C, I564C, I564T, or I564D mutation in the amino acid sequence SEQ ID NO: 2. In some preferred embodiments, the protein effector polypeptide comprises an amino acid sequence comprising a 1564R mutation in the amino acid sequence of SEQ ID NO: 2.

In some embodiments, the protein effector polypeptide comprises an amino acid sequence comprising a mutation at position 565 in the amino acid sequence of SEQ ID NO: 2. For example, the protein effector polypeptide comprises an amino acid sequence comprising a T565R, T565K, T564H, T565C, T565D, or T565G mutation in the amino acid sequence of SEQ ID NO: 2. In some preferred embodiments, the protein effector polypeptide comprises an amino acid sequence comprising a T565R mutation in the amino acid sequence of SEQ ID NO: 2.

In some embodiments, the protein effector polypeptide comprises an amino acid sequence comprising a mutation at one, two, three or four of positions 490, 491, 576 and 577 of the amino acid of SEQ ID NO: 8. For example, the protein effector polypeptide comprises an amino acid sequence comprising a mutation at least at positions 490 and 491, at positions 490 and 577, at positions 490 and 577, at positions 491 and 576, at positions 491 and 577, at positions 577 and 577, at positions 490, 491 and 576, at positions 490, 491 and 577, at positions 490, 576 and 577, at positions 491, 576 and 577, or at positions 490, 491, 576 and 577 of SEQ ID NO: 8.

It is noted that protein effector polypeptide comprising mutations at one, two, three or four of positions 490, 491, 576 and 577 of the amino acid of SEQ ID NO: 8 exhibit increased activity on a target sequence comprising a non-canonical TMA sequence. They are particularly effective on target sequences comprising the TAM sequence RTR, where R is purine (e.g., G or A). In some embodiments, the protein effector polypeptide comprising mutations at one, two, three or four of positions 490, 491, 576 and 577 of the amino acid of SEQ ID NO: 8 exhibit increased activity on a target sequence comprising the TAM sequence ATR. Particularly, the protein effector polypeptide comprising mutations at one, two, three or four of positions 490, 491, 576 and 577 of the amino acid of SEQ ID NO: 8 exhibit increased activity on a target sequence comprising the TAM sequence ATR in human cells.

In some embodiments, the t protein effector polypeptide comprising mutations at one, two, three or four of positions 490, 491, 576 and 577 of the amino acid of SEQ ID NO: 8 exhibits increased activity on a target sequence comprising a TAM sequence selected from the group consisting of RTAAA, ATRAA, ATRMM, ATRA, ATRRA, ATARA, ATRRAC, ATAAAC, ATARM, ATRAM, ATRRM, ATRMA, ATANA, ATRR, ATR, ATGAC, ATAA, ATAR, ATGMC, ATGAA, and ATRAR (where R is purine (e.g., A or G) and M is A) at its 3′ end as compared to SEQ ID NO: 8.

In some embodiments, the protein effector polypeptide comprises an amino acid sequence comprising a mutation at position 490 in the amino acid sequence of SEQ ID NO: 8. For example, the protein effector polypeptide comprises an amino acid sequence comprising a H490N, H490C or H491P mutation in the amino acid sequence of SEQ ID NO: 8. In some preferred embodiments, the protein effector polypeptide comprises an amino acid sequence comprising a H490N mutation in the amino acid sequence of SEQ ID NO: 8.

In some embodiments, the protein effector polypeptide comprises an amino acid sequence comprising a mutation at position 491 in the amino acid sequence of SEQ ID NO: 8. For example, the protein effector polypeptide comprises an amino acid sequence comprising a H491N, H491C or H491M mutation in the amino acid sequence of SEQ ID NO: 8. In some preferred embodiments, the protein effector polypeptide comprises an amino acid sequence comprising a H491N mutation in the amino acid sequence of SEQ ID NO: 8.

In some embodiments, the protein effector polypeptide comprises an amino acid sequence comprising a mutation at position 576 in the amino acid sequence of SEQ ID NO: 8. For example, the protein effector polypeptide comprises an amino acid sequence comprising a I576R, I576K, I576H, I576N, I576Q, I576C, I576C, I576T, or I576D mutation in the amino acid sequence SEQ ID NO: 8. In some preferred embodiments, the protein effector polypeptide comprises an amino acid sequence comprising a I576R mutation in the amino acid sequence of SEQ ID NO: 8.

In some embodiments, the protein effector polypeptide comprises an amino acid sequence comprising a mutation at position 577 in the amino acid sequence of SEQ ID NO: 8. For example, the protein effector polypeptide comprises an amino acid sequence comprising a T577R, T577K, T576H, T577C, T577D, or T577G mutation in the amino acid sequence of SEQ ID NO: 8. In some preferred embodiments, the protein effector polypeptide comprises an amino acid sequence comprising a T577R mutation in the amino acid sequence of SEQ ID NO: 8.

In some embodiments, the protein effector polypeptide comprises an amino acid sequence of SEQ ID No: 2, where at least one, at least two, at least three, at last four, at least five, at least six, at least seven, at least eight, at least nine, or at least ten, at least eleven amino acid sequence between positions 560 to 571 is changed. For example, the protein effector polypeptide comprises an amino acid sequence of SEQ ID No: 2, where at least one, at least two, at least three, at last four, at least five, or at least six amino acid sequence between positions 563 to 568 is changed.

Without wishing to be bound by a theory, protein effector polypeptides comprising a one, two, three, four five or six amino acid sequence swap at positions 563 to 568, or a one, two, three, four five, six, seven, eight, nine, ten or eleven amino acid sequence swap at position 560-571 of SEQ ID NO: 2 exhibit increased activity on a target sequence comprising the TAM sequence RTAAA, where R is purine (e.g., G or A).

In some embodiments, the protein effector polypeptide comprises an amino acid sequence comprising the sequence QHCHGA (SEQ ID NO: 17), NSHYGQ (SEQ ID NO: 18), QHCHGN (SEQ ID NO: 19), QQNKGD (SEQ ID NO: 20), THAKS (SEQ ID NO: 21), QKNNGV (SEQ ID NO: 22), VLNKGA (SEQ ID NO: 23), SSGAGK (SEQ ID NO: 24), NSNGGA (SEQ ID NO: 25), IKNRGR (SEQ ID NO: 26), MHNKGK (SEQ ID NO: 27), VFNYGK (SEQ ID NO: 28), THNKGA (SEQ ID NO: 29), AHGYGK (SEQ ID NO: 30), NKNKGK (SEQ ID NO: 31), THSYGK (SEQ ID NO: 32), MHSYGR (SEQ ID NO: 33), SQNKGT (SEQ ID NO: 34), TTGLGK (SEQ ID NO: 35), KTNN (SEQ ID NO: 36), TQNKGR (SEQ ID NO: 37), THCKGT (SEQ ID NO: 38), QHTNGR (SEQ ID NO: 39), QRTNGN (SEQ ID NO: 40), QRTNGS (SEQ ID NO: 41), QRTRGQ (SEQ ID NO: 42), QASGGK (SEQ ID NO: 43), QSSGGK (SEQ ID NO: 44), QQSNGR (SEQ ID NO: 45), QHSNGA (SEQ ID NO: 46), QKNRGY (SEQ ID NO: 47), QTSGGR (SEQ ID NO: 11), RSSGGK (SEQ ID NO: 12), QQSKGR (SEQ ID NO: 13), QQGRGR (SEQ ID NO: 14), QQNKGR (SEQ ID NO: 15), QLSKGI (SEQ ID NO: 48), QKDKGA (SEQ ID NO: 16), TKNKGT (SEQ ID NO: 49), or GKNGYV (SEQ ID NO: 50) at position Q563-K568 of SEQ ID NO: 2.

Without wishing to be bound by a theory, protein effector polypeptides comprising the sequence QTSGGR (SEQ ID NO: 11), RSSGGK (SEQ ID NO: 12), QQSKGR (SEQ ID NO: 13), QQGRGR (SEQ ID NO: 14), QQNKGR (SEQ ID NO: 15), and QKDKGA (SEQ ID NO: 16) at position 563-568 of SEQ ID NO: 2 exhibit increased activity on a target sequence comprising the TAM sequence RTAAA, where R is purine (e.g., G or A). Accordingly, in some embodiments, the protein polypeptide comprises the sequence QTSGGR (SEQ ID NO: 11), RSSGGK (SEQ ID NO: 12), QQSKGR (SEQ ID NO: 13), QQGRGR (SEQ ID NO: 14), QQNKGR (SEQ ID NO: 15), and QKDKGA (SEQ ID NO: 16) at position 563-568 of SEQ ID NO: 2, and wherein the protein effector polypeptide has editing activity on a target sequence comprising the TAM sequence RTAAA, where R is purine (e.g., G or A) that is higher than the editing activity of protein effector polypeptide comprising the SEQ ID NO: 2. For example, the protein polypeptide comprising the sequence QTSGGR (SEQ ID NO: 11), RSSGGK (SEQ ID NO: 12), QQSKGR (SEQ ID NO: 13), QQGRGR (SEQ ID NO: 14), QQNKGR (SEQ ID NO: 15), and QKDKGA (SEQ ID NO: 16) at position 563-568 of SEQ ID NO: 2 has higher editing activity compared to the editing activity of protein effector polypeptide comprising the SEQ ID NO: 2 when the target sequence comprises a TAM sequence selected from the group consisting of RTAAA, ATRAA, ATRMM, ATRA, ATRRA, ATARA, ATRRAC, ATAAAC, ATARM, ATRAM, ATRRM, ATRMA, ATANA, ATRR, ATR, ATGAC, ATAA, ATAR, ATGMC, ATGAA, and ATRAR (where R is purine (e.g., A or G) and M is A) at its 3′ end.

In some embodiments, the protein effector polypeptide comprises an amino acid sequence comprising the sequence MSGQHCHGAYLR (SEQ ID NO: 51), LSGNSHYGQYYM (SEQ ID NO: 52), MTGQHCHGNYFR (SEQ ID NO: 53), MTGQQNKGDRYF (SEQ ID NO: 54), KSGQLTNGQYFR (SEQ ID NO: 55), LKGNACNGAYFH (SEQ ID NO: 56), SSGTHAKSYFR (SEQ ID NO: 57), LGGQKNNGVYFY (SEQ ID NO: 58), VTGSSGAGKYVR (SEQ ID NO: 59), VRSVHSYGKYVR (SEQ ID NO: 60), VQGIKNRGRYLH (SEQ ID NO: 61), LRGTSSYGKTVL (SEQ ID NO: 62), VKGVFNYGKWVR (SEQ ID NO: 63), VKGKHNDRWI (SEQ ID NO: 64), VKGVQNRGKYIK (SEQ ID NO: 65), IKGSHCNGSRLI (SEQ ID NO: 66), SGGVMSYGRYIT (SEQ ID NO: 67), SKGNKNKGKSIL (SEQ ID NO: 68), AIGTHCKGKRVI (SEQ ID NO: 69), VKGVFNRGTWVR (SEQ ID NO: 70), CKGMHSYGRYVL (SEQ ID NO: 71), IGGTQNKGRYAI (SEQ ID NO: 72), or TKGTHCKGTRIM (SEQ ID NO: 73) at position M560-R571 of SEQ ID NO: 2.

Without wishing to be bound by a theory, protein effector polypeptides comprising one of these sequences at position 560-571 of SEQ ID NO: 2 exhibit increased activity on a target sequence comprising the TAM sequence RTAAA, where R is purine (e.g., G or A). Accordingly, in some embodiments, the protein polypeptide comprises the one of the above sequences at position 650-571 of SEQ ID NO: 2, and wherein the protein effector polypeptide has editing activity on a target sequence comprising the TAM sequence RTAAA, where R is purine (e.g., G or A) that is higher than the editing activity of protein effector polypeptide comprising the SEQ ID NO: 2. For example, the protein polypeptide comprising the one of the above sequences at position 560-571 of SEQ ID NO: 2 has higher editing activity compared to the editing activity of protein effector polypeptide comprising the SEQ ID NO: 2 when the target sequence comprises a TAM sequence selected from the group consisting of RTAAA, ATRAA, ATRMM, ATRA, ATRRA, ATARA, ATRRAC, ATAAAC, ATARM, ATRAM, ATRRM, ATRMA, ATANA, ATRR, ATR, ATGAC, ATAA, ATAR, ATGMC, ATGAA, and ATRAR (where R is purine (e.g., A or G) and M is A) at its 3′ end.

In some embodiments, the protein effector polypeptide comprises an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with a protein effector polypeptide sequence shown in Table B.

TABLE B
Some exemplary protein effector polypeptide sequences (Table discloses
SEQ ID NOS 74-81, respectively, in order of appearance)
Sequence

| | |
|---|---|
| 1 | msidatlirt tpshaeadat dtlvatplmp prrvispwpg pgegqslmri pvvdirgmal |
| 61 | mpctpakarh llksgnarpk rnklglfyvq lsyeqepdnq slvagvdpgs kfeglsvvgt |
| 121 | kdtvlnlmve apdhvkgavq trrtmrrarr qrkwrrpkrf hnrlnrmqri ppstrsrwea |
| 181 | karivahlrt ilpftdvvve dvqavtrkgk ggtwngsfsp vqvgkehlyr llramgltlh |
| 241 | lregwqtkel reqhgikktk skskqsfesh avdswylaas isgaehptct rlwymvpail |
| 301 | hrrqlhrlqa skggvrkpyg gtrslgvkrg tlvehkkygr ctvggvdrkr ntislheyrt |
| 361 | ntrltqaakv etcrvltwls wrswllrgkr tsskgkgshs s |
| 1 | mqpakqqnwv fqingdkqpl dminpgrcre lqnrgklasf rrfpyvviqq qtienpqtke |
| 61 | yilkidpgsq wtgfaiqcgn dilfraelnh rgeaikfdlv krawfrrgrr srnlryrkkr |
| 121 | lntakpegwl apsirhtvlt vetwikrfmr ycpiawieie qvrfdtqkla npeidgveyq |
| 181 | qgelqgyevr eyllqkwgrk caycgtenvp levehiqsks kggssrignl tlachvcnvk |
| 241 | kgnldvrdfl akspdilnqv lenstkplkd aaavnstrya ivkmaksice nvkcssgart |
| 301 | kmntvrqgle kthsldaacv gesgasirvl tdrpllitck ghgsrqsirv nasgfpavkn |
| 361 | aktvfthiaa gdvvrftigk drkkaqagty tarvktptpk gfevlidgar islstmsnvv |
| 421 | fvhrsdgygy cl |
| 1 | mavfvidkhk rplmpcsekr arlllergra vvhrqvpfvi rlkdrtvqhs avqplrvald |
| 61 | pgsratgmal vrekntvdtg tgevyreria lnlfelvhrg hrireqldqr rnfrrrrrga |
| 121 | nlryraprfd arrrppgwla pslqhrvdtt mawvrrlerw apasaigiet vrfdtqrlqn |
| 181 | peisgveyqq galagcevre yllekwgrkc aycgaenvpl eiehivpksr ggsdrvsnla |
| 241 | lacracnqak gnrdvrafla dqperlaril aqakaplkda aavnatrwal yralvdtglp |
| 301 | veagtggrtk wnrtrlglpk thaldalcvg qvdqvrhwrv pvlgircagr gsyntrltr |
| 361 | hgfprgyltr nksafgfqtg dliravvtkg kkagtylgri airasgsfni qtpmgvvqgi |
| 421 | hhrfctliqr adgygyfvqp kpteaalssp rikagvssag n |
| 1 | mttnvvfvid tnqkplqpcs aavarklllr gkaamfrryp aviilkkevd svgkpkielr |
| 61 | idpgskytgf alvdskdnad fiiwgteleh rgaaickelt krsairrsrr nrktryrkkr |
| 121 | ferrkpegwl apslqhrvdt tltwvkrick fvpimsisve qvkfdlqkle nsdiqgieyq |
| 181 | qgtlagytlr callehwgrk cayedvenvf leiehiypks kggsdkfsnl tlachkcnin |
| 241 | kgnksidefl lsdhkrleqi klhqkktlkd aaavnatrkk lvttlqektf lnvlvsdgas |
| 301 | tkmtrlsssl akrhwidage vnttlivilk tlqplqvken ghgnkqfvtm daygfprksy |
| 361 | epkkvrkdwk agdiirvtkk dgtmlmgrvk kaakklvyip fggkeasfss enakaihrsd |
| 421 | gvrysfaaid sellqkmar |
| 1 | mpnkyafvld skgklldptk skkawylirk gkaslveeyp liiklkrevp kdqvnsdkli |
| 61 | lgiddgykkv gfalvqkcqt knkvlfkavm eqrqdvskkm eerrgyrryr rshkryrpar |
| 121 | fdnrssskrk grippsilqk kqailrvvnk lkkyiridki vledvsidir kltegrelyn |
| 181 | weyqesnrld enlrkatlyr ddctcqlcgt tctmlhahhi mprrdggads iynlitlcka |
| 241 | chkdkvdnne yqykdqflai idskelsdlk sashvmqgkt wlrdklskia qleitsggnt |
| 301 | ankridyeie kshsndaict tgllpvdnid dikeyyikpl rkkskakike lkcfrqrdlv |
| 361 | kytkrngety tgyitslrik nnkynskven fstlkgkifr gygfraltll nrpkglmiv |
| 1 | mlfnkciiis inldfsnkek emtkpysigl digtnsvgwa vitdnykvps kkmkvlgnts |
| 61 | kkyikknllg vllfdsgita egrrlkrtar rrytrrrnri lylqeifste matlddaffq |
| 121 | rlddsflvpd dkrdskypif gnlveekvyh defptiyhir kyladstkka dlrlvylala |
| 181 | hmikyrghfl iegefnsknn diqknfqdfl dtynaifesd lslenskqle eivkdkiskl |
| 241 | ekkdrilklf pgeknsgifs eflklivgnq adfrkcfnld ekaslhfske sydedletll |
| 301 | gyigddysdv flkakklyda illsgfltvt dneteaplss amikrynehk edlallkeyi |
| 361 | rnisiktyne vfkddtkngy agyidgktnq edfyvylknl laefegadyf lekidredfl |
| 421 | rkqrtfdngs ipyqihlqem raildkqakf ypflaknker iekilifrip yyvgplargn |
| 481 | sdfawsirkr nekitpwnfe dvidkessae afinrmtsfd lylpeekvlp khsllyetfn |
| 541 | vyneltkvrf iaesmrdyqf ldskqkkdiv rlyfkdkrkv tdkdiieylh aiygydgiel |
| 601 | kgiekqfnss lstyhdllni indkefldds sneaiieeii htltifedre mikqriskfe |
| 661 | nifdksvlkk lsrrhytgwg klsaklingi rdeksgntil dyliddgisn rnfmqlihdd |
| 721 | alsfkkkiqk aqiigdedkg nikevvkslp gspaikkgil qsikivdelv kvmggrkpes |
| 781 | ivvemarenq ytnqgksnsq qrlkrleksl kelgskilke nipaklskid nnalqndrly |
| 841 | lyylqngkdm ytgddldidr lsnydidhii pqaflkdnsi dnkvlvssas nrgksddfps |
| 901 | levvkkrktf wyqllkskli sqrkfdnltk aerggllped kagfiqrqlv etrqitkhva |
| 961 | tlldekfnnk kdennravrt vkiitlkstl vsqfrkdfel ykvreindfh hahdaylnav |
| 1021 | iasallkkyp klepefvygd ypkynsfrer ksatekvyfy snimnifkks isladgrvie |
| 1081 | rplievneet gesvwnkesd latvrrvlsy pqvnvvkkve eqnhgldrgk pkglfnanls |
| 1141 | skpkpnsnen lvgakeyldp kkyggyagis nsfavlvkgt iekgakkkit nvlefqgisi |
| 1201 | ldrinyrkdk lnfllekgyk dieliielpk yslfelsdgs rrmlasilst nnkrgeihkg |
| 1261 | nqiflsqkfv kllyhakris ntinenhrky venhkkefee lfyyilefne nyvgakkngk |
| 1321 | llnsafqswq nhsideless figptgserk glfeltsrgs aadfeflgvk ipryndytps |
| 1381 | sllkdatlih qsvtglyetr idlaklgeg |
| 1 | mkrnyilgld igitsvgygi idyetrdvid agvrlfkean vennegrrsk rgarrlkrrr |
| 61 | rhriqrvkkl lfdynlltdh selsginpye arvkglsqkl seeefsaall hlakrrgvhn |
| 121 | vneveedtgn elstkeqisr nskaleekyv aelglerlkk dgevrgsinr fktsdyvkea |
| 181 | kqllkvqkay hqldqsfidt yidlletrrt yyegpgegsp fgwkdikewy emlmghctyf |
| 241 | peelrsvkya ynadlynaln dlnnlvitrd enekleyyek fqiienvfkq kkkptlkqia |
| 301 | keilvneedi kgyrvtstgk peftnlkvyh dikditarke iienaelldq iakiltiyqs |
| 361 | sediqeeltn lnseltqeei eqisnlkgyt gthnlslkai nlildelwht ndnqiaifnr |
| 421 | lklvpkkvdl sqqkeipttl vddfilspvy krsfiqsikv inaiikkygl pndiiielar |
| 1 | kysigldigt nsvgwavitd eykvpskkfk vlgntdrhsi kknligallf dsgetaeatr |
| 61 | lkrtarrryt rrknricylq eifsnemakv ddsffhrlee sflveedkkh erhpifgniv |
| 121 | devayhekyp tiyhlrkklv dstdkadlrl iylalahmik frghfliegd lnpdnsdvdk |
| 181 | lfiqlvqtyn qlfeenpina sgvdakails arlskstrle nliaqlpgek knglfgnlia |
| 241 | lslgltpnfk snfdlaedak lqlskdtydd dldnllaqig dqyadlflaa knlsdaills |
| 301 | dilrvnteit kaplsasmik rydehhqdlt llkalvrqql pekykeiffd qskngyagyi |
| 361 | dggasqeefy kfikpilekm dgteellvkl nredllrkqr tfdngsiphq ihlgelhail |
| 421 | rrgedfypfl kdnrekieki ltfripyyvg plargnsrfa wmtrkseeri tpwnfeevvd |
| 481 | kgasaqsfie rmtnfdknlp nekvlpkhsl lyeyftvyne ltkvkyvteg mrkpaflsge |
| 541 | qkkaivdllf ktnrkvtvkq lkedyfkkie cfdsveisgv edrinaslgt yhdllkiikd |
| 601 | kdfldneene diledivltl tlfedremie erlktyahlf ddkvmkqlkr rrytgwgrls |
| 661 | rklingirdk qsgktildfl ksdgfanrnf mqlihddslt fkediqkaqv sgqgdslheh |
| 721 | ianlagspai kkgilqtvkv vdelvkvmgr hkpeniviem arenqttqkg qknsrermkr |
| 781 | ieegikelgs qilkehpven tqlqneklyl yylqngrdmy vdqeldinrl sdydvdhivp |
| 841 | qsflkddsid nkvltrsdkn rgksdnvpse evvkkmknyw rqllnaklit qrkfdnltka |
| 901 | ergglseldk agfikrqlve trqitkhvaq ildsrmntky dendklirev kvitlksklv |
| 961 | sdfrkdfqfy kvreinnyhh ahdaylnavv gtalikkypk lesefvygdy kvydvrkmia |
| 1021 | kseqeigkat akyffysnim nffkieitla ngeirkrpli etngetgeiv wdkgrdfatv |
| 1081 | rkvlsmpqvn ivkktevqtg gfskesilpk rnsdkliark kdwdpkkygg fdsptvaysv |
| 1141 | lvvakvekgk skklksvkel lgitimerss feknpidfle akgykevkkd liiklpkysl |
| 1201 | felengrkrm lasagelqkg nelalpskyv nflylashye klkgspedne qkqlfveqhk |
| 1261 | hyldeiieqi sefskrvila danldkvlsa ynkhrdkpir egaeniihlf tltnlgapaa |
| 1321 | fkyfdttidr krytstkevl datlihqsit glyetridls qlggd |

In some embodiments, the protein effector polypeptide comprises an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with an homolog or ortholog of a protein effector polypeptide sequence shown in Table B.

Some exemplary protein effector polypeptides comprising a RuvC and/or HNH domain are described, for example, in PCT publications WO2022087494, WO2020180699, WO2021226363, WO2022261292, WO2021119275, WO2019237069, WO2018107028, WO2019067872 and WO2019067992, content of each of which is incorporated herein by reference in their entireties.

In some embodiments, the protein effector polypeptide comprises an amino acid sequence of SEQ ID No: 8, where at least one, at least two, at least three, at last four, at least five, at least six, at least seven, at least eight, at least nine, or at least ten, at least eleven amino acid sequence between positions 572 to 583 is changed. For example, the protein effector polypeptide comprises an amino acid sequence of SEQ ID No: 8, where at least one, at least two, at least three, at last four, at least five, or at least six amino acid sequence between positions 575 to 580 is changed.

Without wishing to be bound by a theory, protein effector polypeptides comprising a one, two, three, four five or six amino acid sequence swap at positions 575 to 580, or a one, two, three, four five, six, seven, eight, nine, ten or eleven amino acid sequence swap at position 572-583 of SEQ ID NO: 8 exhibit increased activity on a target sequence comprising the TAM sequence RTAAA, where R is purine (e.g., G or A).

In some embodiments, the protein effector polypeptide comprises an amino acid sequence comprising the sequence QHCHGA (SEQ ID NO: 17), NSHYGQ (SEQ ID NO: 18), QHCHGN (SEQ ID NO: 19), QQNKGD (SEQ ID NO: 20), THAKS (SEQ ID NO: 21), QKNNGV (SEQ ID NO: 22), VLNKGA (SEQ ID NO: 23), SSGAGK (SEQ ID NO: 24), NSNGGA (SEQ ID NO: 25), IKNRGR (SEQ ID NO: 26), MHNKGK (SEQ ID NO: 27), VFNYGK (SEQ ID NO: 28), THNKGA (SEQ ID NO: 29), AHGYGK (SEQ ID NO: 30), NKNKGK (SEQ ID NO: 31), THSYGK (SEQ ID NO: 32), MHSYGR (SEQ ID NO: 33), SQNKGT (SEQ ID NO: 34), TTGLGK (SEQ ID NO: 35), KTNN (SEQ ID NO: 36), TQNKGR (SEQ ID NO: 37), THCKGT (SEQ ID NO: 38), QHTNGR (SEQ ID NO: 39), QRTNGN (SEQ ID NO: 40), QRTNGS (SEQ ID NO: 41), QRTRGQ (SEQ ID NO: 42), QASGGK (SEQ ID NO: 43), QSSGGK (SEQ ID NO: 44), QQSNGR (SEQ ID NO: 45), QHSNGA (SEQ ID NO: 46), QKNRGY (SEQ ID NO: 47), QTSGGR (SEQ ID NO: 11), RSSGGK (SEQ ID NO: 12), QQSKGR (SEQ ID NO: 13), QQGRGR (SEQ ID NO: 14), QQNKGR (SEQ ID NO: 15), QLSKGI (SEQ ID NO: 48), QKDKGA (SEQ ID NO: 16), TKNKGT (SEQ ID NO: 49), or GKNGYV (SEQ ID NO: 50) at position Q575-K580 of SEQ ID NO: 8.

Without wishing to be bound by a theory, protein effector polypeptides comprising the sequence QTSGGR (SEQ ID NO: 11), RSSGGK (SEQ ID NO: 12), QQSKGR (SEQ ID NO: 13), QQGRGR (SEQ ID NO: 14), QQNKGR (SEQ ID NO: 15), and QKDKGA (SEQ ID NO: 16) at position 575-580 of SEQ ID NO: 8 exhibit increased activity on a target sequence comprising the TAM sequence RTAAA, where R is purine (e.g., G or A). Accordingly, in some embodiments, the protein polypeptide comprises the sequence QTSGGR (SEQ ID NO: 11), RSSGGK (SEQ ID NO: 12), QQSKGR (SEQ ID NO: 13), QQGRGR (SEQ ID NO: 14), QQNKGR (SEQ ID NO: 15), and QKDKGA (SEQ ID NO: 16) at position 575-580 of SEQ ID NO: 8, and wherein the protein effector polypeptide has editing activity on a target sequence comprising the TAM sequence RTAAA, where R is purine (e.g., G or A) that is higher than the editing activity of protein effector polypeptide comprising the SEQ ID NO: 8. For example, the protein polypeptide comprising the sequence QTSGGR (SEQ ID NO: 11), RSSGGK (SEQ ID NO: 12), QQSKGR (SEQ ID NO: 13), QQGRGR (SEQ ID NO: 14), QQNKGR (SEQ ID NO: 15), and QKDKGA (SEQ ID NO: 16) at position 575-580 of SEQ ID NO: 8 has higher editing activity compared to the editing activity of protein effector polypeptide comprising the SEQ ID NO: 8 when the target sequence comprises a TAM sequence selected from the group consisting of RTAAA, ATRAA, ATRMM, ATRA, ATRRA, ATARA, ATRRAC, ATAAAC, ATARM, ATRAM, ATRRM, ATRMA, ATANA, ATRR, ATR, ATGAC, ATAA, ATAR, ATGMC, ATGAA, and ATRAR (where R is purine (e.g., A or G) and M is A) at its 3′ end.

In some embodiments, the protein effector polypeptide comprises an amino acid sequence comprising the sequence MSGQHCHGAYLR (SEQ ID NO: 51), LSGNSHYGQYYM (SEQ ID NO: 52), MTGQHCHGNYFR (SEQ ID NO: 53), MTGQQNKGDRYF (SEQ ID NO: 54), KSGQLTNGQYFR (SEQ ID NO: 55), LKGNACNGAYFH (SEQ ID NO: 56), SSGTHAKSYFR (SEQ ID NO: 57), LGGQKNNGVYFY (SEQ ID NO: 58), VTGSSGAGKYVR (SEQ ID NO: 59), VRSVHSYGKYVR (SEQ ID NO: 60), VQGIKNRGRYLH (SEQ ID NO: 61), LRGTSSYGKTVL (SEQ ID NO: 62), VKGVFNYGKWVR (SEQ ID NO: 63), VKGKHNDRWI (SEQ ID NO: 64), VKGVQNRGKYIK (SEQ ID NO: 65), IKGSHCNGSRLI (SEQ ID NO: 66), SGGVMSYGRYIT (SEQ ID NO: 67), SKGNKNKGKSIL (SEQ ID NO: 68), AIGTHCKGKRVI (SEQ ID NO: 69), VKGVFNRGTWVR (SEQ ID NO: 70), CKGMHSYGRYVL (SEQ ID NO: 71), IGGTQNKGRYAI (SEQ ID NO: 72), or TKGTHCKGTRIM (SEQ ID NO: 73) at position M572-R583 of SEQ ID NO: 8.

Without wishing to be bound by a theory, protein effector polypeptides comprising one of these sequences at position 572-583 of SEQ ID NO: 8 exhibit increased activity on a target sequence comprising the TAM sequence RTAAA, where R is purine (e.g., G or A). Accordingly, in some embodiments, the protein polypeptide comprises the one of the above sequences at position 650-583 of SEQ ID NO: 8, and wherein the protein effector polypeptide has editing activity on a target sequence comprising the TAM sequence RTAAA, where R is purine (e.g., G or A) that is higher than the editing activity of protein effector polypeptide comprising the SEQ ID NO: 8. For example, the protein polypeptide comprising the one of the above sequences at position 572-583 of SEQ ID NO: 8 has higher editing activity compared to the editing activity of protein effector polypeptide comprising the SEQ ID NO: 8 when the target sequence comprises a TAM sequence selected from the group consisting of RTAAA, ATRAA, ATRMM, ATRA, ATRRA, ATARA, ATRRAC, ATAAAC, ATARM, ATRAM, ATRRM, ATRMA, ATANA, ATRR, ATR, ATGAC, ATAA, ATAR, ATGMC, ATGAA, and ATRAR (where R is purine (e.g., A or G) and M is A) at its 3′ end.

In some embodiments, the protein effector polypeptides described herein are smaller compared to previously identified Cas proteins. Without wishing to be bound by a theory, the protein effector polypeptides and related systems described herein can allow an increased access to the site of target polynucleotide binding, which has several advantages. For example, they can allow easier access to the target polynucleotide for functional domains fused to the protein effector polypeptides or provided in trans. In some embodiments, the RNA: DNA duplex formed by guide molecules that form complex with the protein effector polypeptides is substantially more exposed to the environment and/or functional domains present in proximity of the DNA: RNA complex than the duplexes formed by Cas proteins known in the art. In some embodiments, the protein effector polypeptides confer a different degree of stability of the RNA: DNA duplex. In some embodiments, the protein effector polypeptides enable direct targeting of the DNA: RNA complex by one or more functional domain.

In some embodiments, the protein effector polypeptides and related systems and compositions described herein have no or limited target specificity. For example, a target polynucleotide does not need to have a specific sequence to be targeted by the protein effector polypeptides and related systems and compositions described herein. In some embodiments, the protein effector polypeptides and related systems and compositions described herein do not have a PAM requirement, in that there is no sequence requirement outside of the target sequence which defines target specificity. In some cases, the target specificity of the protein effector polypeptides and related systems and compositions described herein can be determined by the sequence of the guide molecule only, not any sequence within the target polynucleotide. In alternative embodiments, the protein effector polypeptides and related systems and compositions described herein have a target specificity, more particularly the binding of the protein effector polypeptides-guide complex is PAM-dependent. The protein effector polypeptides and related systems and compositions described herein can be modified to include PAM specificity (as described in Kleinstiver et al. 2015; Hirano et al. Mol. Cell 2016).

The protein effector polypeptide and the guide nucleic acid form a complex, also referred to as a surveillance complex. Without wishing to be bound by a theory, the surveillance complex becomes activated upon binding to the target nucleic acid. In some embodiments, the activated complex exhibits “multiple turnover” activity, whereby upon acting on (e.g., cleaving) the target nucleic acid, the activated complex remains in an activated state. In some embodiments, the activated complex exhibits “single turnover” activity, whereby upon acting on the target nucleic acid, the surveillance complex reverts to an inactive state. In some embodiments, the activated complex exhibits non-specific (i.e., “collateral”) cleavage activity, whereby the activated complex cleaves nucleic acids with no sequence similarity to the target (“collateral nucleic acids”). In some embodiments, the collateral nucleic acid substrate is a DNA (e.g., a single-stranded or a double-stranded DNA). In some embodiments, the collateral nucleic acid substrate is an RNA (e.g., a single-stranded or a double-stranded RNA).

While not bound by theory, it is believed that the protein effector polypeptides described herein can act as independent Cas effector and recognize a processed CRISPR array. Alternatively, the protein effector polypeptides described herein interact directly with a Cas enzyme, e.g., Cas12a and bind to a target nucleic acid.

In some embodiments, the protein effector polypeptides described herein stabilize foreign RNA. In some embodiments, the protein effector polypeptides described herein comprise contain tetratricopeptide repeats (TPRs) and a serine-threonine kinase domain. It is believed that the TPRs mediate specific protein-protein interactions with Cas enzyme (e.g., Cas13a.) and Cas enzyme (e.g., Cas13a) recognition of target RNA induces a conformational shift in Cas13a and the kinase domain of the bound protein effector polypeptide. The conformational shift activates the kinase activity leading to phosphorylation of protein substrates and/or autophosphorylation of the kinase. Thus, protein effector polypeptide herein can be leveraged to couple specific RNA detection with phosphorylation of proteins.

### Nuclear Localization Sequences

In some embodiments of any one of the aspects described herein, the protein effector polypeptide further comprises a nuclear localization sequence (NLS) sequence or a variant thereof. The NLS can be proximal to the N- or C-terminus of the protein effector polypeptide. The NLS can be linked (e.g., fused) to the N-terminus and/or C-terminus of the protein effector polypeptide, and can be fused singly (i.e., a single NLS) or concatenated e.g., a chain of 2, 3, 4, or more NLS). When more than one NLS is present, each may be selected independently of the others, such that a single NLS may be present in more than one copy and/or in combination with one or more other NLSs present in one or more copies.

In some embodiments, the protein effector polypeptide is fused to one or more nuclear localization sequences (NLSs), such as about or more than about 1, 2, 3, 4, 5, 6, 7, 8, 9, 10, or more NLSs. In some embodiments, the protein effector polypeptide comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). Some exemplary NLS include, but are not limited to, those shown in Table C.

TABLE C
Exemplary NLS sequences

| Sequence | Source |
|---|---|
| PKKKRKV (SEQ ID NO: 82) | SV40 virus large T- |
| | antigen |
| KRPAATKKAGQAKKKK | nucleoplasmin |
| (SEQ ID NO: 83) | bipartite NLS |
| PAAKRVKLD (SEQ ID NO: 84) | c-myc NLS |
| RQRRNELKRSP (SEQ ID NO: 85) | c-myc NLS |
| NQSSNFGPMKGGNFGGRSSGPYGGGGQY | hRNPA1 M9 NLS |
| FAKPRNQGGY (SEQ ID NO: 86) | |
| RMRIZFKNKGKDTAELRRRRVEVSVELR | IBB domain from |
| KAKKDEQILKRRNV (SEQ ID NO: | importin-alpha |
| 87) | |
| VSRKRPRP (SEQ ID NO: 88) | myoma T protein |
| PPKKARED (SEQ ID NO: 89) | myoma T protein |
| PQPKKKPL (SEQ ID NO: 90) | human p53 |
| SALIKKKKKMAP (SEQ ID NO: 91) | mouse c-ab1 IV |
| DRLRR (SEQ ID NO: 92) | influenza virus NS1 |
| PKQKKRK (SEQ ID NO: 93) | influenza virus NS1 |
| RKLKKKIKKL (SEQ ID NO: 94) | Hepatitis virus |
| | delta antigen |
| REKKKFLKRR (SEQ ID NO: 95) | mouse Mx1 protein |
| KRKGDEVDGVDEVAKKKSKK | human poly (ADP- |
| (SEQ ID NO: 96) | ribose) polymerase |
| RKCLQAGMNLEARKTKK | human glucocorticoid |
| (SEQ ID NO: 97) | receptor |

In some embodiments, the NLS comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with any one of the NLS sequences show in in Table C, or a homolog or ortholog thereof.

### Nuclear Export Signal Sequences

In some embodiments of any one of the aspects described herein, the protein effector polypeptide further comprises a nuclear export signal (NES) sequence or a variant thereof. The NES can be proximal to the N- or C-terminus of the protein effector polypeptide. The NES can be linked (e.g., fused) to the N-terminus and/or C-terminus of the protein effector polypeptide, and can be fused singly (i.e., a single NES) or concatenated e.g., a chain of 2, 3, 4, or more NLS). When more than one NES is present, each may be selected independently of the others, such that a single NES may be present in more than one copy and/or in combination with one or more other NESs present in one or more copies.

In some embodiments, the protein effector polypeptide is fused to one or more NESs, such as about or more than about 1, 2, 3, 4, 5, 6, 7, 8, 9, 10, or more NESs. In some embodiments, the protein effector polypeptide comprises about or more than about 1, 2, 3, 4, 5, 6, 7, 8, 9, 10, or more NESs at or near the amino-terminus, about or more than about 1, 2, 3, 4, 5, 6, 7, 8, 9, 10, or more NESs at or near the carboxy-terminus, or a combination of these (e.g. zero or at least one or more NES at the amino-terminus and zero or at one or more NES at the carboxy terminus).

In some embodiments, a C-terminal and/or N-terminal NLS or NES is attached to the protein effector polypeptide for optimal expression and nuclear targeting in eukaryotic cells, e.g., human cells.

### RuvC Domain

Embodiments of the various aspects described herein include a RuvC domain. As used herein, the term “RuvC domain” refers to a conserved domain or motif of amino acids having nuclease (e.g., endonuclease) activity. A RuvC domain or segments thereof can generally be identified by alignment to known domain sequences, structural alignment to proteins with annotated domains, or by comparison to Hidden Markov Models (HMMs) built based on known domain sequences. Generally, the RuvC domain is comprises of three discontiguous segments or subdomains, RuvC I, RuvC II, and RuvC III. The subdomains can be separated by interval sequences on the amino acid sequence of the polypeptide described herein.

The RuvC-I subdomain can share a structural similarity and/or sequence similarity to a RuvC-I domain described in the art. For example, the RuvC-I subdomain can share a structural similarity and/or sequence similarity to a RuvC-I of Cas9. In some embodiments, the RuvC-I subdomain comprises an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with a homolog or ortholog of RuvC-I of Cas9.

The RuvC-II subdomain can share a structural similarity and/or sequence similarity to a RuvC-II domain described in the art. For example, the RuvC-II subdomain can share a structural similarity and/or sequence similarity to a RuvC-II of Cas9. In some embodiments, the RuvC-II subdomain comprises an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with a homolog or ortholog of RuvC-II of Cas9.

The RuvC-III subdomain can share a structural similarity and/or sequence similarity to a RuvC-III domain described in the art. For example, the RuvC-III subdomain can share a structural similarity and/or sequence similarity to a RuvC-III of Cas9. In some embodiments, the RuvC-III subdomain comprises an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with a homolog or ortholog of a RuvC-III of Cas9.

In some embodiments of any one of the aspects described herein, the RuvC domain is a split RuvC domain. As used herein, a split RuvC domain refers to a RuvC domain having two or more RuvC motifs or subdomains, at sequentially disparate sites within a sequence, that interact in a tertiary structure to form a RuvC domain. Alternatively, the two or more RuvC motifs/subdomains can be present on different polypeptide chains and interact in a tertiary structure to form a RuvC domain.

It is noted that subdomains of the RuvC domain can be from the same species or different species. In some embodiments, the RuvC-I, RuvC-II, and RuvC-III subdomains are from different species. For example, a RuvC domain described herein can comprise a RuvC-I subdomain from a first species, a RuvC-II subdomain from a second species, and a RuvC-II subdomain from a third species. In some other embodiments, two of the RuvC-I, RuvC-II, and RuvC-III subdomains are from the same species and the other is from a different species. For example, a RuvC domain described herein can comprise RuvC-I and RuvC-II subdomains from a first species, a RuvC-III subdomain from a second species; or RuvC-I and RuvC-III subdomains from a first species, a RuvC-II subdomain from a second species; or RuvC-II and RuvC-III subdomains from a first species, a RuvC-I subdomain from a second species.

In some embodiments, the RuvC-I, RuvC-II, and RuvC-III subdomains are from the same species.

The RuvC domain can share a structural similarity and/or sequence similarity to a RuvC domain described in the art. For example, the RuvC domain can share a structural similarity and/or sequence similarity to a RuvC domain of Cas9. In some embodiments, the RuvC domain comprises an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with a homolog or ortholog of a RuvC domain of Cas9.

Some exemplary amino acid sequences for RuvC, RuvC-I, RuvC-II, and RuvC-III domains are described, for example, in WO2022087494, content of which is incorporated herein by reference in its entirety.

### HNH Domain

Embodiments of the various aspects described herein include a HNH domain. As used herein, the term “HNH domain” generally refers to an endonuclease domain having characteristic histidine and asparagine residues. An HNH domain can generally be identified by alignment to known domain sequences, structural alignment to proteins with annotated domains, or by comparison to Hidden Markov Models (HMMs) built based on known domain sequences (e.g., Pfam HMM PF01844 for domain HNH).

The HNH domain can share a structural similarity and/or sequence similarity to a HNH domain described in the art. For example, the HNH domain can share a structural similarity and/or sequence similarity to a HNH domain of Cas9.

In some embodiments, the HNH domain comprises an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with a homolog or ortholog of a HNH domain of Cas9.

In some embodiments, the protein effector polypeptide comprises a RuvC domain (e.g., a split RuvC domain) and a HNH domain. For example, the protein effector polypeptide comprises both a HNH and a split RuvC domain. The HNH domain can be located within the RuvC domain. Alternatively, the HNH domain can be located within the RuvC domain. For example, the HNH domain is located between the Ruv-C II and RuvC-III subdomains of the RuvC domain.

In some embodiments, the protein effector polypeptide comprises a RuvC domain (e.g., a split RuvC domain) but no HNH domain. In some other embodiments, the protein effector polypeptide comprises a HNH domain but no RuvC domain.

Some exemplary amino acid sequences for HNH domain are described, for example, in WO2022087494, content of which is incorporated herein by reference in its entirety.

### Bridge Helix

In some embodiments of any one of the aspects described herein, the RuvC domain is split by the insertion of a bridge helix, a HNH domain, or both. For example, the RuvC domain is split by insertion of a bridge helix. It is noted that the bridge helix can be inserted between the RuvC-I and RuvC-II subdomains, and/or between the RuvC-II and RuvC-III. In some preferred embodiments, the bridge helix is inserted between the RuvC-I and RuvC-II subdomains.

The bridge helix can share a structural similarity and/or sequence similarity to a bridge helix described in the art. For example, the bridge helix can share a structural similarity and/or sequence similarity to a bridge helix of Cas9. In some embodiments, the bridge helix comprises an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with a homolog or ortholog of a bridge helix sequence of Cas9.

### PLMP Domain

In some embodiments of any one of the aspects described herein, the protein effector polypeptide can further comprise a conserved domain (also referred to herein as a PLMP domain), which is not present in Cas9 proteins. The PLMP domain can be located at or near the N terminus of the protein effector polypeptide.

Generally, the PLMP domain is no more than 10, no more than 20, no more than 30, no more than 40, no more than 50, no more than 60, no more than 70, no more than 80, no more than 90, or no more than 100 amino acids in length. For example, the PLMP domain can be no more than 70 amino acids in length. For example, the PLMP domain is 2, 3, 4, 5, 6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 amino acids in length. When present, the PLMP domain can be upstream of the RuvC domain (e.g., RuvC-I subdomain). For example, the PLMP domain is located within 150, 140, 130, 120, 110, 100, 90, 80, 70, 60, 50, 40, 30, 20 or 10 amino acids upstream of the RuvC domain (e.g., RuvC-I subdomain).

Some exemplary amino acid sequences for the PLMP domain are described, for example, in WO2022087494, content of which is incorporated herein by reference in its entirety.

### Domain

In some embodiments of any one of the aspects described herein, the protein effector polypeptide can further comprise a C-terminal domain (also referred to herein as a Y domain herein) located at or near the C-terminus of the protein effector polypeptide.

Generally, the Y domain is no more than 10, no more than 20, no more than 30, no more than 40, no more than 50, no more than 60, no more than 70, no more than 80, no more than 90, or no more than 100 amino acids in length. For example, the Y domain can be no more than 70 amino acids in length. For example, the Y domain is 2, 3, 4, 5, 6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 amino acids in length.

When present, the Y domain can be downstream of the RuvC domain (e.g., RuvC-III subdomain). For example, the Y domain can be located within 150, 140, 130, 120, 110, 100, 90, 80, 70, 60, 50, 40, 30, 20 or 10 amino acids downstream of the RuvC domain (e.g., RuvC-III subdomain).

Some exemplary amino acid sequences for the PLMP domain are described, for example, in WO2022087494, content of which is incorporated herein by reference in its entirety.

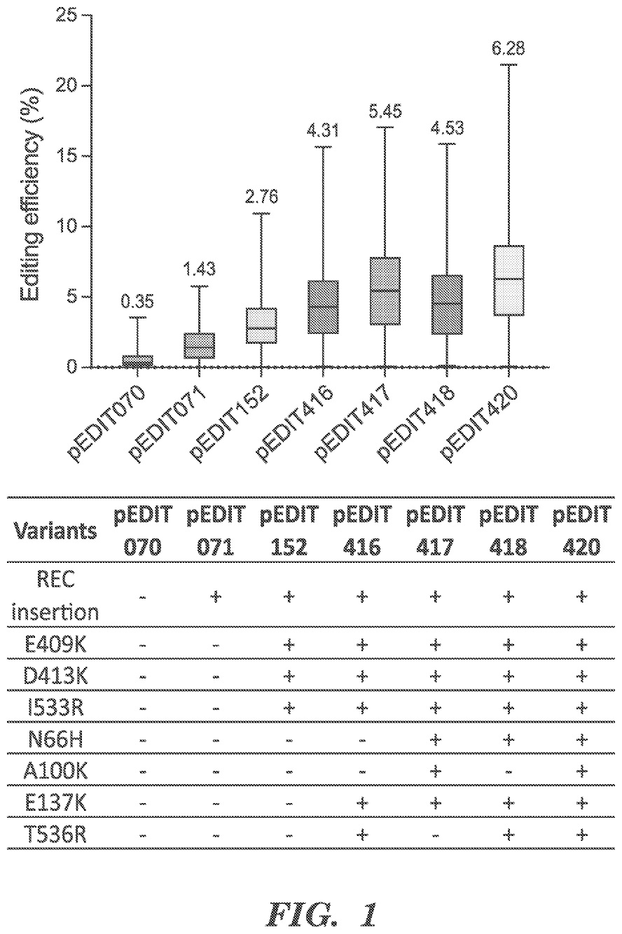

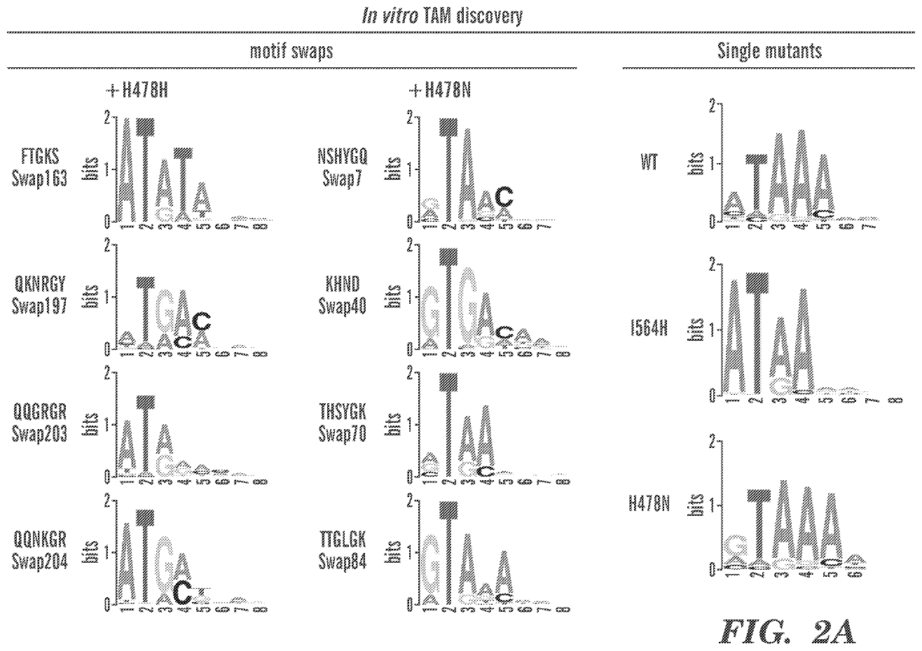

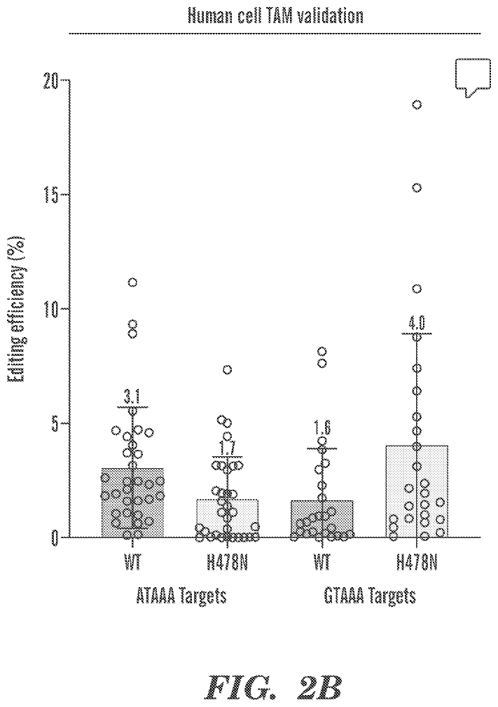

In some embodiments of any one of the aspects described herein, the protein effector polypeptide comprises a C-terminal domain that is structurally homologous to a tudor domain. See, for example, Ren et al., Cell Res. (2014) 24:1146-1149. Tudor domains typically comprise a barrel-shaped beta strand fold and range in size around 50 and 60 amino acids. See, for example, Kawale, A. A. & Burmann, B. M. Inherent backbone dynamics fine-tune the functional plasticity of Tudor domains. Structure (2021), incorporated herein by reference; see, in particular, FIG. 1 of Kawale showing exemplary tudor domain structure.

### Recognition (REC) Domain

In some embodiments of any one of the aspects described herein, the protein effector polypeptide further comprises a recognition (REC) domain. The REC domain can linked/fused to the N-terminal or the C-terminal of the protein effector polypeptide. Accordingly, in some embodiments, the REC domain is linked/fused to the N-terminal protein effector polypeptide. In some other embodiments, the REC domain is linked/fused to the C-terminal protein effector polypeptide. In some embodiments, the REC domain is at an internal position of the effector polypeptide.

The REC domain can share a structural similarity and/or sequence similarity to a REC domain described in the art. For example, the REC domain can share a structural similarity and/or sequence similarity to REC domain of Cas9 or a homolog or ortholog thereof. In some embodiments, the REC domain comprises an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with a homolog or ortholog of a REC of Cas9 or a homolog or ortholog thereof.

In some embodiments, the REC domain comprises an amino acid sequence having at least about 85%, at least about 90%, at least about 91%, at least about 92%, at least about 93%, at least about 94%, at least about 95%, at least about 96%, at least about 97%, at least about 98%, at least about 99%, or 100% identity with the sequence:

(SEQ ID NO: 9)
ILTKCKVCGKNTPLRRNVRELLLENIVRFLPLESELKETLKRTILEGQQG
NINKLFRKLKFNQKDWPGKNLTDIAKNKLPGRLPFCKEHFAENEKFTTIE
KSTFR.

In some embodiments, the REC domain comprises an amino acid sequence at least about 85%, at least about 90%, at least about 91%, at least about 92%, at least about 93%, at least about 94%, at least about 95%, at least about 96%, at least about 97%, at least about 98%, at least about 99%, or 100% identity a REC domain of any one of SEQ ID NOs: 1-198, 221-459, 463-612, or 617-668 described in WO2021202568, content of which is incorporated herein by reference.

In some embodiments, the REC domain can be linked/fused to the N-terminal or the C-terminal of the protein effector polypeptide via a linker comprising one or more amino acids, i.e., a peptide linker.

### Functional Mutations of Protein Effector Polypeptides

Various mutations or modifications can be introduced into protein effector polypeptides as described herein to improve specificity and/or robustness. In some embodiments, the amino acid residues that recognize the Target Adjacent Motif (TAM) are identified. The protein effector polypeptides described herein can be modified further to recognize different TAMs, e.g., by substituting the amino acid residues that recognize PAM with other amino acid residues.

In some embodiments, the amino acid sequence of the protein effector polypeptide is mutated at one or more amino acid residues to alter its ability to functionally associate with a guide nucleic acid. In some embodiments, the protein effector polypeptide is mutated at one or more amino acid residues to alter its ability to functionally associate with a target nucleic acid. In some embodiments, the protein effector polypeptide described herein are capable of binding to or modifying a target nucleic acid molecule. In some embodiments, the protein effector polypeptide modifies both strands of the target nucleic acid molecule. However, in some embodiments, the protein effector polypeptide is mutated at one or more amino acid residues to alter its nucleic acid manipulation activity. For example, in some embodiments, the protein effector polypeptide can comprise one or more mutations which render the protein effector polypeptide incapable of cleaving a target nucleic acid. In other embodiments, the protein effector polypeptide can comprise one or more mutations such that the protein effector polypeptide is capable of cleaving a single strand of the target nucleic acid (i.e., nickase activity). In some embodiments, the protein effector polypeptide is capable of cleaving the strand of the target nucleic acid that is complementary to the strand to which the guide nucleic acid hybridizes. In some embodiments, the protein effector polypeptide is capable of cleaving the strand of the target nucleic acid to which the guide nucleic acid hybridizes.

In some embodiments, a protein effector polypeptide described herein may be engineered to comprise a deletion in one or more amino acid residues to reduce the size while retaining one or more desired functional activities (e.g., nuclease activity and the ability to interact functionally with a guide nucleic acid). The truncated protein effector polypeptide may be advantageously used in combination with delivery systems having load limitations.

### Functional Domains

The protein effector polypeptides described herein have nuclease activity. However, they can be modified to have reduced nuclease activity, e.g., nuclease inactivation of at least 50%, at least 60%, at least 70%, at least 80%, at least 90%, at least 95%, at least 97%, or 100% as compared with the wild-type sequence. The nuclease activity can be reduced by, for example, introducing mutations (such as amino acid insertions, deletions, or substitutions) into the nuclease domains of the protein effector polypeptides described herein. In some embodiments, catalytic residues for the nuclease activities are identified, and these amino acid residues can be substituted by different amino acid residues (e.g., glycine or alanine) to diminish the nuclease activity.

The inactivated protein effector polypeptides can comprise (e.g., via fusion protein, linker peptides, Gly4Ser (SEQ ID NO: 98) (GS) peptide linkers, etc.) or be associated (e.g., via co-expression of multiple proteins) with one or more functional domains. These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, and switch activity (e.g., light inducible). Accordingly, in some embodiments of any one of the aspects described herein, the protein effector polypeptide can be associated with a functional domain (also referred to as an effector domain herein), e.g., a domain having transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, and/or detectable activity. For example, the protein effector polypeptide further comprises a functional domain having transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, and/or detectable activity.

In some embodiments, the functional domain is a ligase or a functional fragment thereof. In some embodiments, the functional domain is a deaminase or a functional fragment thereof. In some other embodiments, the functional domain is a transposase or a functional fragment thereof. In yet some other embodiments, the functional domain is a reverse transcriptase or a functional fragment thereof. In some embodiments, the functional domain is a transcriptional activation domain or a functional fragment thereof. For example, the functional domain is VP64, VP16, p65, MyoD1, HSF1, RTA, SET7/9, a histone acetyltransferase, or a functional fragment thereof. In some embodiments, the functional domain is a transcription repression domain or a functional fragment thereof. For example, the functional domain is Krüppel associated box (KRAB), SID, or concatemers of SID (e.g., SID4X). In some embodiments, the functional domain is an epigenetic modifying domain or a functional fragment thereof. In some embodiments, the functional domain can be an activation domain or a functional fragment thereof, such as the P65 activation domain.

In some embodiments, the functional domains are selected from the group consisting of Krüppel associated box (KRAB), VP64, VP16, Fok1, P65, HSF1, MyoD1, biotin-APEX, and functional fragments of any one of them.

The functional domain can be operably coupled to the protein effector polypeptide. One or more functional domains can be tethered or linked via a suitable linker (including, but not limited to, Gly Ser linkers) to the protein effector polypeptide. When there is more than one functional domain, the functional domains can be same or different. In some embodiments, all the functional domains are the same. In some embodiments, all of the functional domains are different from each other. In embodiments, at least two of the functional domains are different from each other. In some embodiments, at least two of the functional domains are the same as each other.

Generally, each functional domain is in a spatial orientation allowing for the functional domain to function in its attributed function. In other words, the positioning of the one or more functional domains on the inactivated protein effector polypeptides described herein allows for correct spatial orientation for the functional domain to affect the target with the attributed functional effect. For example, if the functional domain is a transcription activator (e.g., VP 16, VP64, or p65), the transcription activator is placed in a spatial orientation that allows it to affect the transcription of the target. Likewise, a transcription repressor (e.g., KRAB) is positioned to affect the transcription of the target, and a nuclease (e.g., Fok1) is positioned to cleave or partially cleave the target. In some embodiments, the functional domain is positioned at the N-terminus of the protein effector polypeptides described herein. In some embodiments, the functional domain is positioned at the C-terminus of the protein effector polypeptides described herein. In some embodiments, the inactivated protein effector polypeptides described herein is modified to comprise a first functional domain at the N-terminus and a second functional domain at the C-terminus.

### Fusion Protein

In another aspect provided herein is a fusion protein comprising a protein effector polypeptide described herein covalently linked to a functional domain. For example, the fusion protein comprises a protein effector polypeptide described herein covalently linked to a functional domain having transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, and/or detectable activity. It is noted that the functional domain can be linked/fused to the N-terminal protein effector polypeptide. In some other embodiments, the functional domain is linked/fused to the C-terminal protein effector polypeptide. In some embodiments, the functional domain can be linked/fused to the N-terminal or the C-terminal of the protein effector polypeptide via a linker comprising one or more amino acids, i.e., a peptidyl linker.

In some embodiments, the fusion protein comprises a functional domain that is a nucleic acid editing domain.

In some embodiments, the fusion protein comprises a functional domain that is a reverse transcriptase or a functional fragment thereof.

In some embodiments, the fusion protein comprises a functional domain that is a deaminase domain or a functional fragment thereof. The term “deaminase” or “deaminase domain,” as used herein, refers to a protein or enzyme that catalyzes a deamination reaction. In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism, that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

In some embodiments, the deaminase or deaminase domain is a cytidine deaminase. A cytidine deaminase domain may also be referred to interchangeably as a cytosine deaminase domain. In some embodiments, the cytidine deaminase catalyzes the hydrolytic deamination of cytidine (C) or deoxycytidine (dC) to uridine (U) or deoxyuridine (dU), respectively. In some embodiments, the cytidine deaminase domain catalyzes the hydrolytic deamination of cytosine (C) to uracil (U). In some embodiments, the cytidine deaminase catalyzes the hydrolytic deamination of cytidine or cytosine in deoxyribonucleic acid (DNA). Without wishing to be bound by any particular theory, fusion proteins comprising a cytidine deaminase are useful inter alia for targeted editing, referred to herein as “base editing,” of nucleic acid sequences in vitro and in vivo.

In some embodiments, the cytidine deaminase or cytidine deaminase domain is a naturally-occurring cytidine deaminase from an organism, such as a human, chimpanzee, gorilla, monkey, cow, dog, rat, or mouse. In some embodiments, the cytidine deaminase or cytidine deaminase domain is a variant of a naturally-occurring cytidine deaminase from an organism that does not occur in nature. For example, in some embodiments, the cytidine deaminase or cytidine deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring cytidine deaminase from an organism, such as a human, chimpanzee, gorilla, monkey, cow, dog, rat, or mouse

In some embodiments, the cytidine deaminase is an apolipoprotein B mRNA-editing complex (APOBEC) family deaminase. In some embodiments, the cytidine deaminase is an APOBEC1 deaminase. In some embodiments, the cytidine deaminase is an APOBEC2 deaminase. In some embodiments, the cytidine deaminase is an APOBEC3 deaminase. In some embodiments, the cytidine deaminase is an APOBEC3A deaminase. In some embodiments, the cytidine deaminase is an APOBEC3B deaminase. In some embodiments, the cytidine deaminase is an APOBEC3C deaminase. In some embodiments, the cytidine deaminase is an APOBEC3D deaminase. In some embodiments, the cytidine deaminase is an APOBEC3E deaminase. In some embodiments, the cytidine deaminase is an APOBEC3F deaminase. In some embodiments, the cytidine deaminase is an APOBEC3G deaminase. In some embodiments, the cytidine deaminase is an APOBEC3H deaminase. In some embodiments, the cytidine deaminase is an APOBEC4 deaminase. In some embodiments, the cytidine deaminase is an activation-induced deaminase (AID). In some embodiments, the cytidine deaminase is a vertebrate cytidine deaminase. In some embodiments, the cytidine deaminase is an invertebrate cytidine deaminase. In some embodiments, the cytidine deaminase is a human, chimpanzee, gorilla, monkey, cow, dog, rat, or mouse deaminase. In some embodiments, the cytidine deaminase is a human cytidine deaminase. In some embodiments, the cytidine deaminase is a rat cytidine deaminase, e.g., rAPOBEC1. In some embodiments, the cytidine deaminase is a Petromyzon marinus cytidine deaminase 1 (pmCDA1). In some embodiments, the cytidine deaminase is a human APOBEC3G. In some embodiments, the cytidine deaminase is a fragment of the human APOBEC3G. In some embodiments, the deaminase is a human APOBEC3G variant comprising a D316R and D317R mutation. In some embodiments, the deaminase is a fragment of the human APOBEC3G and comprising mutations corresponding to the D316R and D317R mutations.

In some embodiments, the cytidine deaminase domain is at least 80%, at least 85%, at least 90%, at least 92%, at least 95%, at least 96%, at least 97%, at least 98%, at least 99%, or at least 99.5% identical to the cytidine deaminase domain of any one of the exemplary cytidines deaminase sequences shown in Table D1. In some embodiments, the cytidine deaminase domain comprises the amino acid sequence of any one of the exemplary cytidines deaminase sequences shown in Table D1. It should be understood that, in some embodiments, the active domain of the respective sequence can be used, e.g., the domain without a localizing signal (nuclear localization sequence, without nuclear export signal, cytoplasmic localizing signal).

TABLE D1
Exemplary cytidine deaminase sequences

| SEQUENCE | Type/Organism |
|---|---|
| MDSLLMNRRKFLYQFKNVRW AKGRRETYLCYVVKRRDSAT | AID [Homo |
| SFSLDFGYLRNKNGCHVELL FLRYISDWDLDPGRCYRVTW | sapiens] |
| FTSWSPCYDCARHVADFLRG | |
| NPNLSLRIFTARLYFCEDRKAEPEGLRRLHRAGVQIAIMT | |
| FKDYFYCWNTFVENHERTFK AWEGLHENSVRLSRQLRRIL | |
| LPLYEVDDLRDAFRTLGL (SEQ ID NO: 99) | |
| MDSLLMKQKK FLYHFKNVRW AKGRHETYLC YVVKRRDSAT | AID [Mus |
| SCSLDFGHLR NKSGCHVELL FLRYISDWDL DPGRCYRVTW | musculus] |
| FTSWSPCYDC ARHVAEFLRW NPNLSLRIFT ARLYFCEDRKAEPEGLRRLH | |
| RAGVQIGIMT FKDYFYCWNT FVENRERTFK AWEGLHENSV | |
| RLTRQLRRIL LPLYEVDDLR DAFRMLGF (SEQ ID NO: 100) | |
| MDSLLMKQRK FLYHFKNVRW AKGRHETYLC YVVKRRDSAT | AID [Canis |
| SFSLDFGHLR NKSGCHVELL FLRYISDWDL DPGRCYRVTW | familiaris] |
| FTSWSPCYDC ARHVADFLRG YPNLSLRIFA ARLYFCEDRKAEPEGLRRLH | |
| RAGVQIAIMT FKDYFYCWNT FVENREKTFK AWEGLHENSV | |
| RLSRQLRRIL LPLYEVDDLR DAFRTLGL (SEQ ID NO: 101) | |
| MDSLLKKQRQ FLYQFKNVRW AKGRHETYLC YVVKRRDSPT | AID [Bos |
| SFSLDFGHLR NKAGCHVELL FLRYISDWDL DPGRCYRVTW | taurus] |
| FTSWSPCYDC ARHVADFLRG YPNLSLRIFT ARLYFCDKERKAEPEGLRRL | |
| HRAGVQIAIM TFKDYFYCWN TFVENHERTF KAWEGLHENS | |
| VRLSRQLRRI LLPLYEVDDL RDAFRTLGL (SEQ ID NO: 102) | |
| MAVGSKPKAA LVGPHWERER IWCFLCSTGL GTQQTGQTSR | AID [Rattus] |
| WLRPAATQDP VSPPRSLLMK QRKFLYHFKN VRWAKGRHET | |
| YLCYVVKRRD SATSFSLDFG YLRNKSGCHV ELLFLRYISDWDLDPGRCYR | |
| VTWFTSWSPC YDCARHVADF LRGNPNLSLR IFTARLTGWG | |
| ALPAGLMSPA RPSDYFYCWN TFVENHERTF KAWEGLHENS | |
| VRLSRRLRRI LLPLYEVDDL RDAFRTLGL (SEQ ID NO: 103) | |
| MGPFCLGCSH RKCYSPIRNL ISQETFKFHF KNLGYAKGRK DTFLCYEVTR | ApoBEC-3 |
| KDCDSPVSLH HGVFKNKDNI HAEICFLYWF HDKVLKVLSP | [Mus musculus] |
| REEFKITWYM SWSPCFECAE QIVRFLATHHNLSLDIFSSR LYNVQDPETQ | |
| QNLCRLVQEG AQVAAMDLYE FKKCWKKFVD | |
| NGGRRFRPWKRLLTNFRYQD SKLQEILRPC YIPVPSSSSS TLSNICLTKG | |
| LPETRFCVEG RRMDPLSEEEFYSQFYNQRV KHLCYYHRMK | |
| PYLCYQLEQF NGQAPLKGCL LSEKGKQHAE ILFLDKIRSMELSQVTITCY | |
| LTWSPCPNCA WQLAAFKRDR PDLILHIYTS RLYFHWKRPF | |
| QKGLCSLWQSGILVDVMDLP QFTDCWTNFV NPKRPFWPWK | |
| GLEIISRRTQ RRLRRIKESW GLQDLVNDFGNLQLGPPMS (SEQ ID NO: 104) | |
| MGPFCLGCSH RKCYSPIRNL ISQETFKFHF KNLRYAIDRK DTFLCYEVTR | APOBEC-3 |
| KDCDSPVSLHHGVFKNKDNI HAEICFLYWF HDKVLKVLSP REEFKITWYM | [Rattus] |
| SWSPCFECAE QVLRFLATHHNLSLDIFSSR LYNIRDPENQ QNLCRLVQEG | |
| AQVAAMDLYE FKKCWKKFVD NGGRRFRPWKKLLTNFRYQD | |
| SKLQEILRPC YIPVPSSSSS TLSNICLTKG LPETRFCVER | |
| RRVHLLSEEEFYSQFYNQRV KHLCYYHGVK PYLCYQLEQF | |
| NGQAPLKGCL LSEKGKQHAE ILFLDKIRSMELSQVIITCY LTWSPCPNCA | |
| WQLAAFKRDR PDLILHIYTS RLYFHWKRPF QKGLCSLWQSGILVDVMDLP | |
| QFTDCWTNFV NPKRPFWPWK GLEIISRRTQ RRLHRIKESW | |
| GLQDLVNDFGNLQLGPPMS (SEQ ID NO: 105) | |
| MVEPMDPRTF VSNFNNRPIL SGLNTVWLCC EVKTKDPSGP PLDAKIFQGK | APOBEC-3G |
| VYSKAKYHPEMRFLRWFHKW RQLHHDQEYK VTWYVSWSPC | [Macaca |
| TRCANSVATF LAKDPKVTLT IFVARLYYFWKPDYQQALRI | mulatta] |
| LCQKRGGPHA TMKIMNYNEF QDCWNKFVDG RGKPFKPRNN | |
| LPKHYTLLQATLGELLRHLM DPGTFTSNFN NKPWVSGQHE | |
| TYLCYKVERL HNDTWVPLNQ HRGFLRNQAPNIHGFPKGRH AELCFLDLIP | |
| FWKLDGQQYR VTCFTSWSPC FSCAQEMAKF ISNNEHVSLCIFAARIYDDQ | |
| GRYQEGLRAL HRDGAKIAMM NYSEFEYCWD TFVDRQGRPF | |
| QPWDGLDEHSQALSGRLRAI (SEQ ID NO: 106) | |
| MKPHFRNPVE RMYQDTFSDN FYNRPILSHR NTVWLCYEVK | APOBEC-3G |
| TKGPSRPPLD AKIFRGQVYSKLKYHPEMRF FHWFSKWRKL | [Pan |
| HRDQEYEVTW YISWSPCTKC TRDVATFLAE | troglodytes] |
| DPKVTLTIFVARLYYFWDPD YQEALRSLCQ KRDGPRATMK | |
| IMNYDEFQHC WSKFVYSQRE LFEPWNNLPKYYILLHIMLG EILRHSMDPP | |
| TFTSNFNNEL WVRGRHETYL CYEVERLHND | |
| TWVLLNQRRGFLCNQAPHKH GFLEGRHAEL CFLDVIPFWK | |
| LDLHQDYRVT CFTSWSPCFS CAQEMAKFISNNKHVSLCIF AARIYDDQGR | |
| CQEGLRTLAK AGAKISIMTY SEFKHCWDTF | |
| VDHQGCPFQPWDGLEEHSQA LSGRLRAILQ NQGN (SEQ ID NO: 107) | |
| MNPQIRNMVE QMEPDIFVYY FNNRPILSGR NTVWLCYEVK | APOBEC- |
| TKDPSGPPLD ANIFOGKLYPEAKDHPEMKF LHWFRKWRQL | 3G[Chlorocebus |
| HRDQEYEVTW YVSWSPCTRC ANSVATFLAE | sabaeus] |
| DPKVTLTIFVARLYYFWKPD YQQALRILCQ ERGGPHATMK | |
| IMNYNEFQHC WNEFVDGQGK PFKPRKNLPKHYTLLHATLG | |
| ELLRHVMDPG TFTSNFNNKP WVSGQRETYL CYKVERSHND | |
| TWVLLNQHRGFLRNQAPDRH GFPKGRHAEL CFLDLIPFWK | |
| LDDQQYRVTC FTSWSPCFSC AQKMAKFISNNKHVSLCIFA ARIYDDQGRC | |
| QEGLRTLHRD GAKIAVMNYS EFEYCWDTFV | |
| DRQGRPFQPWDGLDEHSQAL SGRLRAI (SEQ ID NO: 108) | |
| MKPHFRNTVE RMYRDTFSYN FYNRPILSRR NTVWLCYEVK | APOBEC-3G |
| TKGPSRPPLD AKIFRGQVYSELKYHPEMRF FHWFSKWRKL | [Homo sapiens] |
| HRDQEYEVTW YISWSPCTKC TRDMATFLAE | |
| DPKVTLTIFVARLYYFWDPD YQEALRSLCQ KRDGPRATMK | |
| IMNYDEFQHC WSKFVYSQRE LFEPWNNLPKYYILLHIMLG EILRHSMDPP | |
| TFTFNFNNEP WVRGRHETYL CYEVERMHND | |
| TWVLLNQRRGFLCNQAPHKH GFLEGRHAEL CFLDVIPFWK | |
| LDLDQDYRVT CFTSWSPCFS CAQEMAKFISKNKHVSLCIF TARIYDDQGR | |
| CQEGLRTLAE AGAKISIMTY SEFKHCWDTF | |
| VDHQGCPFQPWDGLDEHSQD LSGRLRAILQ NQEN (SEQ ID NO: 109) | |
| MKPHFRNTVE RMYRDTFSYN FYNRPILSRR NTVWLCYEVK | APOBEC-3F |
| TKGPSRPRLD AKIFRGQVYSQPEHHAEMCF LSWFCGNQLP AYKCFQITWF | [Homo sapiens] |
| VSWTPCPDCV AKLAEFLAEH PNVTLTISAARLYYYWERDY | |
| RRALCRLSQA GARVKIMDDE EFAYCWENFV YSEGQPFMPW | |
| YKFDDNYAFLHRTLKEILRN PMEAMYPHIF YFHFKNLRKA | |
| YGRNESWLCF TMEVVKHHSP VSWKRGVFRNQVDPETHCHA | |
| ERCFLSWFCD DILSPNTNYE VTWYTSWSPC PECAGEVAEF | |
| LARHSNVNLTIFTARLYYFW DTDYQEGLRS LSQEGASVEI | |
| MGYKDFKYCW ENFVYNDDEP FKPWKGLKYNFLFLDSKLQE ILE (SEQ ID | |
| NO: 110) | |
| MNPQIRNPME RMYRDTFYDN FENEPILYGR SYTWLCYEVK | APOBEC-3B |
| IKRGRSNLLW DTGVFRGQVYFKPQYHAEMC FLSWFCGNQL | [Homo sapiens] |
| PAYKCFQITW FVSWTPCPDC VAKLAEFLSE HPNVTLTISAARLYYYWERD | |
| YRRALCRLSQ AGARVTIMDY EEFAYCWENF VYNEGQQFMP | |
| WYKFDENYAFLHRTLKEILR YLMDPDTFTF NFNNDPLVLR | |
| RRQTYLCYEV ERLDNGTWVL MDQHMGFLCNEAKNLLCGFY | |
| GRHAELRFLD LVPSLQLDPA QIYRVTWFIS WSPCFSWGCA | |
| GEVRAFLQENTHVRLRIFAA RIYDYDPLYK EALQMLRDAG | |
| AQVSIMTYDE FEYCWDTFVY RQGCPFQPWDGLEEHSQALS | |
| GRLRAILQNQ GN (SEQ ID NO: 111) | |
| MQPQGLGPNA GMGPVCLGCS HRRPYSPIRN PLKKLYQQTF | APOBEC- |
| YFHFKNVRYA WGRKNNFLCYEVNGMDCALP VPLRQGVFRK | 3B[Rattus] |
| QGHIHAELCF IYWFHDKVLR VLSPMEEFKV | |
| TWYMSWSPCSKCAEQVARFL AAHRNLSLAI FSSRLYYYLR | |
| NPNYQQKLCR LIQEGVHVAA MDLPEFKKCWNKFVDNDGQP | |
| FRPWMRLRIN FSFYDCKLQE IFSRMNLLRE DVFYLQFNNS | |
| HRVKPVQNRYYRRKSYLCYQ LERANGQEPL KGYLLYKKGE | |
| QHVEILFLEK MRSMELSQVR ITCYLTWSPCPNCARQLAAF KKDHPDLILR | |
| IYTSRLYFYW RKKFQKGLCT LWRSGIHVDV | |
| MDLPQFADCWTNFVNPQRPF RPWNELEKNS WRIQRRLRRI KESWGL | |
| (SEQ ID NO: 112) | |
| DGWEVAFRSG TVLKAGVLGV SMTEGWAGSG HPGQGACVWT | APOBEC-3B |
| PGTRNTMNLL REVLFKQQFGNQPRVPAPYY RRKTYLCYQL | [Bos taurus] |
| KORNDLTLDR GCFRNKKQRH AEIRFIDKIN SLDLNPSQSYKIICYITWSP | |
| CPNCANELVN FITRNNHLKL EIFASRLYFH WIKSFKMGLQ | |
| DLQNAGISVAVMTHTEFEDC WEQFVDNQSR PFQPWDKLEQ | |
| YSASIRRRLQ RILTAPI (SEQ ID NO: 113) | |
| MNPQIRNPME WMYQRTFYYN FENEPILYGR SYTWLCYEVK | APOBEC-3B |
| IRRGHSNLLW DTGVFRGQMYSQPEHHAEMC FLSWFCGNQL | [Pan |
| SAYKCFQITW FVSWTPCPDC VAKLAKFLAE | troglodytes] |
| HPNVTLTISAARLYYYWERD YRRALCRLSQ AGARVKIMDD | |
| EEFAYCWENF VYNEGQPFMP WYKFDDNYAFLHRTLKEIIR | |
| HLMDPDTFTF NFNNDPLVLR RHQTYLCYEV ERLDNGTWVL | |
| MDQHMGFLCNEAKNLLCGFY GRHAELRFLD LVPSLQLDPA | |
| QIYRVTWFIS WSPCFSWGCA GQVRAFLQENTHVRLRIFAA RIYDYDPLYK | |
| EALQMLRDAG AQVSIMTYDE FEYCWDTFVY | |
| RQGCPFQPWDGLEEHSQALS GRLRAILQVR ASSLCMVPHR PPPPPQSPGP | |
| CLPLCSEPPL GSLLPTGRPAPSLPFLLTAS FSFPPPASLP PLPSLSLSPG | |
| HLPVPSFHSL TSCSIQPPCS SRIRETEGWASVSKEGRDLG (SEQ ID NO: 114) | |
| MNPQIRNPMK AMYPGTFYFQ FKNLWEANDR NETWLCFTVE | APOBEC-3C |
| GIKRRSVVSW KTGVFRNQVDSETHCHAERC FLSWFCDDIL | [Homo sapiens] |
| SPNTKYQVTW YTSWSPCPDC AGEVAEFLAR HSNVNLTIFTARLYYFQYPC | |
| YQEGLRSLSQ EGVAVEIMDY EDFKYCWENF VYNDNEPFKP | |
| WKGLKTNFRLLKRRLRESLQ (SEQ ID NO: 115) | |
| MNPQIRNPMK AMYPGTFYFQ FKNLWEANDR NETWLCFTVE | Gorilla |
| GIKRRSVVSW KTGVFRNQVDSETHCHAERC FLSWFCDDIL | APOBEC3C |
| SPNTNYQVTW YTSWSPCPEC AGEVAEFLAR HSNVNLTIFTARLYYFQDTD | [Gorilla |
| YQEGLRSLSQ EGVAVKIMDY KDFKYCWENF VYNDDEPFKP | gorilla] |
| WKGLKYNFRFLKRRLQEILE (SEQ ID NO: 116) | |
| MEASPASGPR HLMDPHIFTS NFNNGIGRHK TYLCYEVERL | APOBEC-3A |
| DNGTSVKMDQ HRGFLHNQAKNLLCGFYGRH AELRFLDLVP | [Homo sapiens] |
| SLQLDPAQIY RVTWFISWSP CFSWGCAGEV RAFLQENTHVRLRIFAARIY | |
| DYDPLYKEAL QMLRDAGAQV SIMTYDEFKH CWDTFVDHQG | |
| CPFQPWDGLDEHSQALSGRL RAILQNQGN (SEQ ID NO: 117) | |
| MDGSPASRPR HLMDPNTFTF NFNNDLSVRG RHQTYLCYEV | APOBEC-3A |
| ERLDNGTWVP MDERRGFLCNKAKNVPCGDY GCHVELRFLC | [Macaca |
| EVPSWQLDPA QTYRVTWFIS WSPCFRRGCA | mulatta] |
| GQVRVFLQENKHVRLRIFAA RIYDYDPLYQ EALRTLRDAG AQVSIMTYEE | |
| FKHCWDTFVD RQGRPFQPWDGLDEHSQALS GRLRAILQNQ GN (SEQ ID | |
| NO: 118) | |
| MDEYTFTENF NNQGWPSKTY LCYEMERLDG DATIPLDEYK | APOBEC-3A |
| GFVRNKGLDQ PEKPCHAELYFLGKIHSWNL DRNQHYRLTC FISWSPCYDC | [Bos taurus] |
| AQKLTTFLKE NHHISLHILA SRIYTHNRFGCHQSGLCELQ AAGARITIMT | |
| FEDFKHCWET FVDHKGKPFQ PWEGLNVKSQ ALCTELQAILKTQQN (SEQ | |
| ID NO: 119) | |
| MALLTAETFR LQFNNKRRLR RPYYPRKALL CYQLTPQNGS | APOBEC-3H |
| TPTRGYFENK KKCHAEICFINEIKSMGLDE TQCYQVTCYL TWSPCSSCAW | [Homo sapiens] |
| ELVDFIKAHD HLNLGIFASR LYYHWCKPQQKGLRLLCGSQ | |
| VPVEVMGFPK FADCWENFVD HEKPLSFNPY KMLEELDKNS | |
| RAIKRRLERIKIPGVRAQGR YMDILCDAEV (SEQ ID NO: 120) | |
| MALLTAKTFS LQFNNKRRVN KPYYPRKALL CYQLTPQNGS | APOBEC-3H |
| TPTRGHLKNK KKDHAEIRFINKIKSMGLDE TQCYQVTCYL TWSPCPSCAG | [Macaca |
| ELVDFIKAHR HLNLRIFASR LYYHWRPNYQEGLLLLCGSQ VPVEVMGLPE | mulatta] |
| FTDCWENFVD HKEPPSFNPS EKLEELDKNS QAIKRRLERIKSRSVDVLEN | |
| GLRSLQLGPV TPSSSIRNSR (SEQ ID NO: 121) | |
| MNPQIRNPME RMYRDTFYDN FENEPILYGR SYTWLCYEVK | APOBEC-3D |
| IKRGRSNLLW DTGVFRGPVLPKRQSNHRQE VYFRFENHAE | [Homo sapiens] |
| MCFLSWFCGN RLPANRRFQI TWFVSWNPCL PCVVKVTKFLAEHPNVTLTI | |
| SAARLYYYRD RDWRWVLLRL HKAGARVKIM DYEDFAYCWE | |
| NFVCNEGQPFMPWYKFDDNY ASLHRTLKEI LRNPMEAMYP | |
| HIFYFHFKNL LKACGRNESW LCFTMEVTKHHSAVFRKRGV | |
| FRNQVDPETH CHAERCFLSW FCDDILSPNT NYEVTWYTSW | |
| SPCPECAGEVAEFLARHSNV NLTIFTARLC YFWDTDYQEG LCSLSQEGAS | |
| VKIMGYKDFV SCWKNFVYSDDEPFKPWKGL QTNFRLLKRR LREILQ | |
| (SEQ ID NO: 122) | |
| MTSEKGPSTG DPTLRRRIEP WEFDVFYDPR ELRKEACLLY EIKWGMSRKI | APOBEC-1 |
| WRSSGKNTTNHVEVNFIKKF TSERDFHPSM SCSITWFLSW SPCWECSQAI | [Homo sapiens] |
| REFLSRHPGV TLVIYVARLFWHMDQQNRQG LRDLVNSGVT | |
| IQIMRASEYY HCWRNFVNYP PGDEAHWPQY PPLWMMLYALELHCIILSLP | |
| PCLKISRRWQ NHLTFFRLHL QNCHYQTIPP HILLATGLIH PSVAWR (SEQ | |
| ID NO: 123) | |
| MSSETGPVAV DPTLRRRIEP HEFEVFFDPR ELRKETCLLY EINWGGRHSV | APOBEC-1 |
| WRHTSQNTSNHVEVNFLEKF TTERYFRPNT RCSITWFLSW SPCGECSRAI | [Mus musculus] |
| TEFLSRHPYV TLFIYIARLYHHTDQRNRQG LRDLISSGVT IQIMTEQEYC | |
| YCWRNFVNYP PSNEAYWPRY PHLWVKLYVLELYCIILGLP PCLKILRRKQ | |
| PQLTFFTITL QTCHYQRIPP HLLWATGLK (SEQ ID NO: 124) | |
| MSSETGPVAV DPTLRRRIEP HEFEVFFDPR ELRKETCLLY EINWGGRHSI | APOBEC-1 |
| WRHTSQNTNKHVEVNFIEKF TTERYFCPNT RCSITWFLSW SPCGECSRAI | [Rattus] |
| TEFLSRYPHV TLFIYIARLYHHADPRNRQG LRDLISSGVT IQIMTEQESG | |
| YCWRNFVNYS PSNEAHWPRY PHLWVRLYVLELYCIILGLP PCLNILRRKQ | |
| PQLTFFTIAL QSCHYQRLPP HILWATGLK (SEQ ID NO: 125) | |
| MAQKEEAAVA TEAASQNGED LENLDDPEKL KELIELPPFE IVTGERLPAN | APOBEC-2 |
| FFKFQFRNVEYSSGRNKTFL CYVVEAQGKG GQVQASRGYL | [Homo sapiens] |
| EDEHAAAHAE EAFFNTILPA FDPALRYNVTWYVSSSPCAA CADRIIKTLS | |
| KTKNLRLLIL VGRLFMWEEP EIQAALKKLK | |
| EAGCKLRIMKPQDFEYVWQN FVEQEEGESK AFQPWEDIQE | |
| NFLYYEEKLA DILK (SEQ ID NO: 126) | |
| MAQKEEAAEA AAPASQNGDD LENLEDPEKL KELIDLPPFE IVTGVRLPVN | APOBEC-2 |
| FFKFQFRNVEYSSGRNKTFL CYVVEVQSKG GQAQATQGYL | [Mus musculus] |
| EDEHAGAHAE EAFFNTILPA FDPALKYNVTWYVSSSPCAA CADRILKTLS | |
| KTKNLRLLIL VSRLFMWEEP EVQAALKKLK | |
| EAGCKLRIMKPQDFEYIWQN FVEQEEGESK AFEPWEDIQE NFLYYEEKLA | |
| DILK (SEQ ID NO: 127) | |
| MAQKEEAAEA AAPASQNGDD LENLEDPEKL KELIDLPPFE IVTGVRLPVN | APOBEC-2 |
| FFKFQFRNVEYSSGRNKTFL CYVVEAQSKG GQVQATQGYL | [Rattus] |
| EDEHAGAHAE EAFFNTILPA FDPALKYNVTWYVSSSPCAA CADRILKTLS | |
| KTKNLRLLIL VSRLFMWEEP EVQAALKKLK | |
| EAGCKLRIMKPQDFEYLWQN FVEQEEGESK AFEPWEDIQE NFLYYEEKLA | |
| DILK (SEQ ID NO: 128) | |
| MAQKEEAAAA AEPASQNGEE VENLEDPEKL KELIELPPFE IVTGERLPAH | APOBEC-2 |
| YFKFQFRNVEYSSGRNKTFL CYVVEAQSKG GQVQASRGYL | [Bos taurus] |
| EDEHATNHAE EAFFNSIMPT FDPALRYMVTWYVSSSPCAA CADRIVKTLN | |
| KTKNLRLLIL VGRLFMWEEP EIQAALRKLK EAGCRLRIMKPQDFEYIWQN | |
| FVEQEEGESK AFEPWEDIQE NFLYYEEKLA DILK (SEQ ID NO: 129) | |
| MAQKEEAAAA AEPASQNGEE VENLEDPEKL KELIELPPFE IVTGERLPAH | CDA1 |
| YFKFQFRNVEYSSGRNKTFL CYVVEAQSKG GQVQASRGYL | [Petromyzon |
| EDEHATNHAE EAFFNSIMPT FDPALRYMVTWYVSSSPCAA CADRIVKTLN | marinus] |
| KTKNLRLLIL VGRLFMWEEP EIQAALRKLK EAGCRLRIMKPQDFEYIWQN | |
| FVEQEEGESK AFEPWEDIQE NFLYYEEKLA DILK (SEQ ID NO: 438) | |
| MKPHFRNTVE RMYRDTFSYN FYNRPILSRR NTVWLCYEVK | APOBEC3GD3 |
| TKGPSRPPLD AKIFRGQVYSELKYHPEMRF FHWFSKWRKL | 16R_D317R |
| HRDQEYEVTW YISWSPCTKC TRDMATFLAE | [Homo sapiens] |
| DPKVTLTIFVARLYYFWDPD YQEALRSLCQ KRDGPRATMK | |
| IMNYDEFQHC WSKFVYSQRE LFEPWNNLPKYYILLHIMLG EILRHSMDPP | |
| TFTFNFNNEP WVRGRHETYL CYEVERMHND | |
| TWVLLNQRRGFLCNQAPHKH GFLEGRHAEL CFLDVIPFWK | |
| LDLDQDYRVT CFTSWSPCFS CAQEMAKFISKNKHVSLCIF TARIYRRQGR | |
| CQEGLRTLAE AGAKISIMTY SEFKHCWDTF | |
| VDHQGCPFQPWDGLDEHSQD LSGRLRAILQ NQEN (SEQ ID NO: 130) | |
| MDPPTFTFNF NNEPWVRGRH ETYLCYEVER MHNDTWVLLN | APOBEC3G |
| QRRGFLCNQA PHKHGFLEGRHAELCFLDVI PFWKLDLDQD | [Homo sapiens] |
| YRVTCFTSWS PCFSCAQEMA KFISKNKHVS LCIFTARIYDDQGRCQEGLR | |
| TLAEAGAKIS IMTYSEFKHC WDTFVDHQGC PFQPWDGLDE | |
| HSQDLSGRLRAILQ (SEQ ID NO: 131) | |
| MDPPTFTFNF NNEPWVRGRH ETYLCYEVER MHNDTWVLLN | APOBEC3GD1 |
| QRRGFLCNQA PHKHGFLEGRHAELCFLDVI PFWKLDLDQD | 20R_D121R |
| YRVTCFTSWS PCFSCAQEMA KFISKNKHVS LCIFTARIYRRQGRCQEGLR | [Homo sapiens] |
| TLAEAGAKIS IMTYSEFKHC WDTFVDHQGC PFQPWDGLDE | |
| HSQDLSGRLRAILQ (SEQ ID NO: 132) | |

In another example, the fusion protein comprises a functional domain that is an adenosine deaminase domain or a functional fragment thereof. In some embodiments, the deaminase or deaminase domain is an adenosine deaminase, which catalyzes the hydrolytic deamination of adenine or adenosine. In some embodiments, the deaminase or deaminase domain is an adenosine deaminase, catalyzing the hydrolytic deamination of adenosine or deoxyadenosine to inosine or deoxyinosine, respectively. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of the exemplary adenosine deaminase sequences shown in Table D2, or to any of the adenosine deaminases provided herein. It should be appreciated that adenosine deaminases provided herein may include one or more mutations (e.g., any of the mutations provided herein). In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs: 62-84, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences set forth in in any one of the exemplary adenosine deaminase sequences shown in Table D2, or any of the adenosine deaminases provided herein.

TABLE D2
Exemplary adenosine deaminase sequences

| SEQUENCE | Type/organism |
|---|---|
| EACKALGTWR LEGATLYVTL EPCPMCAGAV VLSRVEKVVF | TadA |
| GAFDPKGGCS GTLMNLLQEERFNHQAEVVS GVLEEECGGM | [Bacillus |
| LSAFFRELRK KKKAARKNLS E (SEQ ID NO: 133) | subtilis] |
| MTQDELYMKE AIKEAKKAEE KGEVPIGAVL VINGEIIARA HNLRETEQRS | TadA |
| IAHAEMLVIDEACKALGTWR LEGATLYVTL EPCPMCAGAV | [Bacillus |
| VLSRVEKVVF GAFDPKGGCS GTLMNLLQEERFNHQAEVVS | subtilis] |
| GVLEEECGGM LSAFFRELRK KKKAARKNLS E (SEQ ID NO: 134) | |
| MSEVEFSHEY WMRHALTLAK RAWDEREVPV GAVLVHNNRV | TadA, |
| IGEGWNRPIG RHDPTAHAAIMALRQGGLVM QNYRLIDATL | catalytically |
| YVTLEPCVMC AGAMIHSRIG RVVFGARDAK | inactive |
| TGAAGSLMDVLHHPGMNHRV EITEGILADE CAALLSDFFR | (E59A)[E. |
| MRRQEIKAQK KAQSSTD (SEQ ID NO: 135) | coli] |
| MSEVEFSHEY WMRHALTLAK RARDEREVPV GAVLVLNNRV | TadA |
| IGEGWNRAIG LHDPTAHAEIMALRQGGLVM QNYRLIDATL | 7.10[Synthetic] |
| YVTFEPCVMC AGAMIHSRIG RVVFGVRNAK | |
| TGAAGSLMDVLHYPGMNHRV EITEGILADE CAALLCYFFR | |
| MPRQVFNAQK KAQSSTD (SEQ ID NO: 136) | |
| MSEVEFSHEY WMRHALTLAK RARDEREVPV GAVLVLNNRV | TadA |
| IGEGWNRAIG LHDPTAHAEIMALRQGGLVM QNYRLIDATL | 7.10(V106W) |
| YVTFEPCVMC AGAMIHSRIG RVVFGWRNAK | [Synthetic] |
| TGAAGSLMDVLHYPGMNHRV EITEGILADE CAALLCYFFR | |
| MPRQVFNAQK KAQSSTD (SEQ ID NO: 137) | |
| MSEVEFSHEY WMRHALTLAK RARDEREVPV GAVLVLNNRV | TadA |
| IGEGWNRAIG LHDPTAHAEIMALRQGGLVM QNYRLIDATL | 7.10(N108W |
| YVTFEPCVMC AGAMIHSRIG RVVFGVRWAK | [Synthetic] |
| TGAAGSLMDVLHYPGMNHRV EITEGILADE CAALLCYFFR | |
| MPRQVFNAQK KAQSSTD (SEQ ID NO: 138) | |
| MSEVEFSHEY WMRHALTLAK RARDEREVPV GAVLVLNNRV | TadA |
| IGEGWNRAIG LHDPTAHAEIMALRQGGLVM QNYRLIDATL | 7.10(N108Q) |
| YVTFEPCVMC AGAMIHSRIG RVVFGVRQAK | [Synthetic] |
| TGAAGSLMDVLHYPGMNHRV EITEGILADE CAALLCYFFR | |
| MPRQVFNAQK KAQSSTD (SEQ ID NO: 139) | |
| MSEVEFSHEY WMRHALTLAK RARDEREVPV GAVLVLNNRV | TadA |
| IGEGWNRAIG LHDPTAHAEIMALRQGGLVM QNYRLIDATL | 7.10(V106F) |
| YVTFEPCVMC AGAMIHSRIG RVVFGFRNAK | [Synthetic] |
| TGAAGSLMDVLHYPGMNHRV EITEGILADE CAALLCYFFR | |
| MPRQVFNAQK KAQSSTD (SEQ ID NO: 140) | |
| MSEVEFSHEY WMRHALTLAK RARDEREVPV GAVLVLNNRV | tadA 7.10 |
| IGEGWNRAIG LHDPTAHAEIMALRQGGLVM QNYRLIDATL | (V106Q) |
| YVTFEPCVMC AGAMIHSRIG RVVFGQRNAK | [Synthetic] |
| TGAAGSLMDVLHYPGMNHRV EITEGILADE CAALLCYFFR | |
| MPRQVFNAQK KAQSSTD (SEQ ID NO: 141) | |
| MSEVEFSHEY WMRHALTLAK RARDEREVPV GAVLVLNNRV | tadA 7.10 |
| IGEGWNRAIG LHDPTAHAEIMALRQGGLVM QNYRLIDATL | (V106M) |
| YVTFEPCVMC AGAMIHSRIG RVVFGMRNAK | [Synthetic] |
| TGAAGSLMDVLHYPGMNHRV EITEGILADE CAALLCYFFR | |
| MPRQVFNAQK KAQSSTD (SEQ ID NO: 142) | |
| MSEVEFSHEY WMRHALTLAK RARDEREVPV GAVLVLNNRV | tadA |
| IGEGWNFAIG LHDPTAHAEIMALRQGGLVM QNYRLIDATL | 7.10(R47F) |
| YVTFEPCVMC AGAMIHSRIG RVVFGVRNAK | [Synthetic] |
| TGAAGSLMDVLHYPGMNHRV EITEGILADE CAALLCYFFR | |
| MPRQVFNAQK KAQSSTD (SEQ ID NO: 143) | |
| MSEVEFSHEY WMRHALTLAK RARDEREVPV GAVLVLNNRV | tadA |
| IGEGWNWAIG LHDPTAHAEIMALRQGGLVM QNYRLIDATL | 7.10(R47W) |
| YVTFEPCVMC AGAMIHSRIG RVVFGVRNAK | [Synthetic] |
| TGAAGSLMDVLHYPGMNHRV EITEGILADE CAALLCYFFR | |
| MPRQVFNAQK KAQSSTD (SEQ ID NO: 144) | |
| MSEVEFSHEY WMRHALTLAK RARDEREVPV GAVLVLNNRV | tadA |
| IGEGWNQAIG LHDPTAHAEIMALRQGGLVM QNYRLIDATL | 7.10(R47Q) |
| YVTFEPCVMC AGAMIHSRIG RVVFGVRNAK | [Synthetic] |
| TGAAGSLMDVLHYPGMNHRV EITEGILADE CAALLCYFFR | |
| MPRQVFNAQK KAQSSTD (SEQ ID NO: 145) | |
| MSEVEFSHEY WMRHALTLAK RARDEREVPV GAVLVLNNRV | tadA |
| IGEGWNMAIG LHDPTAHAEIMALRQGGLVM QNYRLIDATL | 7.10(R47M) |
| YVTFEPCVMC AGAMIHSRIG RVVFGVRNAK | [Synthetic] |
| TGAAGSLMDVLHYPGMNHRV EITEGILADE CAALLCYFFR | |
| MPRQVFNAQK KAQSSTD (SEQ ID NO: 146) | |
| MSEVEFSHEY WMRHALTLAK RAWDEREVPV GAVL VHNNRV | TadA(E59Q) |
| IGEGWNRPIG RHDPTAHAQIMALRQGGLVM QNYRLIDATL | [Synthetic] |
| YVTLEPCVMC AGAMIHSRIG RVVFGARDAK | |
| TGAAGSLMDVLHHPGMNHRV EITEGILADE CAALLSDFFR | |
| MRRQEIKAQK KAQSSTD (SEQ ID NO: 147) | |
| MSEVEFSHEY WMRHALTLAK RAWDEREVPV GAVLVHNNRV | TadA, N- |
| IGEGWNRPIG RHDPTAHAEIMALRQGGLVM QNYRLIDATL | terminal |
| YVTLEPCVMC AGAMIHSRIG RVVFGARDAK | truncated |
| TGAAGSLMDVLHHPGMNHRV EITEGILADE CAALLSDFFR | [E. coli] |
| MRRQEIKAQK KAQSSTD (SEQ ID NO: 148) | |
| MRRAFITGVF FLSEVEFSHE YWMRHALTLA KRAWDEREVP | TadA [E. |
| VGAVLVHNNR VIGEGWNRPIGRHDPTAHAE IMALRQGGLV | coli] |
| MQNYRLIDAT LYVTLEPCVM CAGAMIHSRI | |
| GRVVFGARDAKTGAAGSLMD VLHHPGMNHR VEITEGILAD | |
| ECAALLSDFF RMRRQEIKAQ KKAQSSTD (SEQ ID NO: 149) | |
| MGSHMTNDIY FMTLAIEEAK KAAQLGEVPI GAIITKDDEV IARAHNLRET | Tad |
| LQQPTAHAEHIAIERAAKVL GSWRLEGCTL YVTLEPCVMC AGTIVMSRIP | [Staphylococcus |
| RVVYGADDPK GGCSGSLMNLLQQSNFNHRA IVDKGVLKEA | aureus] |
| CSTLLTTFFK NLRANKKSTN (SEQ ID NO: 150) | |
| MPPAFITGVT SLSDVELDHE YWMRHALTLA KRAWDEREVP | TadA |
| VGAVLVHNHR VIGEGWNRPIGRHDPTAHAE IMALRQGGLV | [S. typhimurium] |
| LQNYRLLDTT LYVTLEPCVM CAGAMVHSRI | |
| GRVVFGARDAKTGAAGSLID VLHHPGMNHR VEIIEGVLRD ECATLLSDFF | |
| RMRRQEIKAL KKADRAEGAGPAV (SEQ ID NO: 151) | |
| MDEYWMQVAM QMAEKAEAAG EVPVGAVLVK DGQQIATGYN | TadA |
| LSISQHDPTA HAEILCLRSAGKKLENYRLL DATLYITLEP CAMCAGAMVH | [S. putrefaciens] |
| SRIARVVYGA RDEKTGAAGT VVNLLQHPAFNHQVEVTSGV | |
| LAEACSAQLS RFFKRRRDEK KALKLAQRAQ QGIE (SEQ ID NO: 152) | |
| MDAAKVRSEF DEKMMRYALE LADKAEALGE IPVGAVLVDD | TadA |
| ARNIIGEGWN LSIVQSDPTAHAEIIALRNG AKNIQNYRLL NSTLYVTLEP | [H. influenza] |
| CTMCAGAILH SRIKRLVFGA SDYKTGAIGSRFHFFDDYKM NHTLEITSGV | |
| LAEECSQKLS TFFQKRREEK KIEKALLKSL SDK (SEQ ID NO: 153) | |

In some cases, the adenosine deaminase is double-stranded RNA-specific adenosine deaminase (ADAR). Examples of ADARs include those described Yiannis A Savva et al., The ADAR protein family, Genome Biol. 2012; 13 (12): 252, which is incorporated by reference in its entirety. In some examples, the ADAR may be hADAR1. In certain examples, the ADAR may be hADAR2. The sequence of hADAR2 may be that described under Accession No. AF525422.1.

In some cases, the deaminase may be a deaminase domain, e.g., a deaminase domain of ADAR (“ADAR-D”). In one example, the deaminase may be the deaminase domain of hADAR2 (“hADAR2-D), e.g., as described in Phelps K J et al., Recognition of duplex RNA by the deaminase domain of the RNA editing enzyme ADAR2. Nucleic Acids Res. 2015 January; 43 (2): 1123-32, which is incorporated by reference herein in its entirety. In a particular example, the hADAR2-D has a sequence comprising amino acid 299-701 of hADAR2-D, e.g., amino acid 299-701 of the sequence under Accession No. AF525422.1. In certain examples, the adenosine deaminase may comprise one or more of the mutations: E488Q based on amino acid sequence positions of hADAR2-D, and mutations in a homologous ADAR protein corresponding to the above. In one embodiment, the adenosine deaminase may comprise one or more of the mutations: E488Q, V351G, based on amino acid sequence positions of hADAR2-D, and mutations in a homologous ADAR protein corresponding to the above. In one embodiment, the adenosine deaminase may comprise one or more of the mutations: E488Q, V351G, S486A, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, V440I based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, V440I, S495N based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, V440I, S495N, K418E based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, V440I, S495N, K418E, S661T based on amino acid sequence positions of hADAR2-D, and mutations in a homologous ADAR protein corresponding to the above. In some examples, provided herein includes a mutated adenosine deaminase e.g., an adenosine deaminase comprising one or more mutations of E488Q, V351G, S486A, T375S, S370C, P462A, N597I, L332I, I398V, K350I, M383L, D619G, S582T, V440I, S495N, K418E, S661T. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, and S661T. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, S661T, and S375N. In one embodiment, the adenosine deaminase may be a tRNA-specific adenosine deaminase or a variant thereof. In one embodiment, the adenosine deaminase may comprise one or more of the mutations: W23L, W23R, R26G, H36L, N37S, P48S, P48T, P48A, I49V, R51L, N72D, L84F, S97C, A106V, D108N, H123Y, G125A, A142N, S146C, D147Y, R152H, R152P, E155V, I156F, K157N, K161T, based on amino acid sequence positions of E. coli TadA, and mutations in a homologous deaminase protein corresponding to the above. In one embodiment, the adenosine deaminase may comprise one or more of the mutations: D108N based on amino acid sequence positions of E. coli TadA, and mutations in a homologous deaminase protein corresponding to the above. In one embodiment, the adenosine deaminase may comprise one or more of the mutations: A1 06V, D108N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, based on amino acid sequence positions of E. coli TadA, and mutations in a homologous deaminase protein corresponding to the above. In one embodiment, the adenosine deaminase may comprise one or more of the mutations: A106V, D108N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, based on amino acid sequence positions of E. coli TadA, and mutations in a homologous deaminase protein corresponding to the above. In one embodiment, the adenosine deaminase may comprise one or more of the mutations: A106V, D108N, D147Y, E1 55V, L84F, H123Y, I156F, H36L, R51L, S146C, K157N, P48S, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, R152P, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, R152P, A142N, based on amino acid sequence positions of E. coli TadA, and mutations in a homologous deaminase protein corresponding to the above.

In some embodiments, the fusion protein further comprises a second adenosine deaminase domain. The second adenosine deaminase can be an ecTadA domain, a variant, or a functional fragment thereof. In some embodiments, the first and second adenosine deaminase domain independently comprises a nucleotide sequence having at least 80%, at least 85%, at least 90%, at least 92%, at least 95%, at least 96%, at least 97%, at least 98%, at least 99%, at least 99.5%, or 100% identity (e.g., when translated into a polypeptide) to any one of to any one of the sequences in Table D2. In one example, both the first and second adenosine deaminase domains are an ecTadA domain, a variant, or a functional fragment thereof.

In some embodiments, the fusion protein comprises a functional domain that is uracil glycosylase inhibitor (UGI) domain or a functional fragment thereof.

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in Table E. In some embodiments, the UGI proteins provided herein include fragments of UGI and proteins homologous to a UGI or a UGI fragment. For example, in some embodiments, a UGI domain comprises a fragment of the amino acid sequence set forth in Table E. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in TABLE E. In some embodiments, a UGI comprises an amino acid sequence homologous to the amino acid sequence set forth in TABLE E, or an amino acid sequence homologous to a fragment of the amino acid sequence set forth in TABLE E. In some embodiments, proteins comprising UGI or fragments of UGI or homologs of UGI or UGI fragments are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in Table E. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in Table E.

TABLE E
Exemplary UGI sequences

| SEQUENCE | Type/organism |
|---|---|
| MTNLSDIIEK ETGKQLVIQE SILMLPEEVE EVIGNKPESD ILVHTAYDES | UNGI_BPPB2 |
| TDENVMLLTSDAPEYKPWAL VIQDSNGENK IKML (SEQ ID NO: 154) | [Bacillus |
| | phagePBS2 |
| KGHLTRLGLE FFDQPAVPLA RAFLGQVLVR RLPNGTELRG | Truncated |
| RIVETQAYLG PEDEAAHSRGGRQTPRNRGM FMKPGTLYVY | AAG |
| IIYGMYFCMN ISSQGDGACV LLRALEPLEG | nuclease(E125Q) |
| LETMRQLRSTLRKGTASRVL KDRELCSGPS KLCQALAINK | [H. sapiens] |
| SFDQRDLAQD | |
| EAVWLERGPLEPSEPAVVAAARVGVGHAGEWARKPLRFYVRGSPWVSV | |
| VD RVAEQDTQA (SEQ ID NO: 155) | |
| DLASLRAQQI ELASSVIRED RLDKDPPDLI AGAAVGFEQG | Endo V |
| GEVTRAAMVL LKYPSLELVE YKVARIATTM PYIPGFLSFR | nuclease(D35A) |
| EYPALLAAWE MLSQKPDLVF VDGHGISHPR | [Synthetic] |
| RLGVASHFGLLVDVPTIGVA KKRLCGKFEP LSSEPGALAP | |
| LMDKGEQLAW VWRSKARCNP LFIATGHRVS VDSALAWVQR | |
| CMKGYRLPEP TRWADAVASE RPAFVRYTANQP (SEQ ID NO: 156) | |
| MASRGVNKVILVGNLGQDPEVRYMPNGGAVANITLATSESWRDKQTGE | SSB |
| TKEKTEWHRVVLFGKLAEVAGEYLRKGSQVYIEGALQTRKWTDQAGVE | [Erwinia |
| KYTTEVVVNVGGTMQMLGGRSQGGGASAGGQNGGSNNGWGQPQQPQ | tasmaniensis] |
| GGNQFSGGAQQQARPQQQPQQNNAPANNEPPIDFDDDIP (SEQ ID NO: 157) | |
| MAGAQDFVPHTADLAELAAAAGECRGCGLYRDATQAVFGAGGRSARIM | UdgX |
| MIGEQPGDKEDLAGLPFVGPAGRLLDRALEAADIDRDALYVTNAVKHFK | [Synthetic] |
| FTRAAGGKRRIHKTPSRTEVVACRPWLIAEMTSVEPDVVVLLGATAAKA | |
| LLGNDFRVTQHRGEVLHVDDVPGDPALVATVHPSSLLRGPKEERESAFA | |
| GLVDDLRVAADVRP (SEQ ID NO: 158) | |
| MIGQKTLYSFFSPSPARKRHAPSPEPAVQGTGVAGVPEESGDAAAIPAKK | UDG |
| APAGQEEPGTPPSSPLSAEQLDRIQRNKAAALLRLAARNVPVGFGESWKK | [Synthetic] |
| HLSGEFGKPYFIKLMGFVAEERKHYTVYPPPHQVFTWTQMCDIKDVKVV | |
| ILGQEPYHGPNQAHGLCFSVQRPVPPPPSLENIYKELSTDIEDFVHPGHGD | |
| LSGWAKQGVLLLNAVLTVRAHQANSHKERGWEQFTDAVVSWLNQNSN | |
| GLVFLLWGSYAQKKGSAIDRKRHHVLQTAHPSPLSVYRGFFGCRHFSKT | |
| NELLQKSGKKPIDWKEL (SEQ ID NO: 159) | |

In yet another example, the fusion protein comprises a functional domain that is reverse transcriptase domain or a functional fragment thereof. A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Retroviral RT has three sequential biochemical activities: RNA-dependent DNA polymerase activity, ribonuclease H, and DNA-dependent DNA polymerase activity. Collectively, these activities enable the enzyme to convert single-stranded RNA into double-stranded cDNA. In an embodiment, the RT domain of a reverse transcriptase is used in the present invention. The domain may include only the RNA-dependent DNA polymerase activity. In some examples, the RT domain is non-mutagenic, i.e., does not cause mutation in the donor polynucleotide (e.g., during the reverse transcriptase process). In some examples, the RT domain may be non-retron RT, e.g., a viral RT or a human endogenous RTs. In some examples, the RT domain may be retron RT or DGRs RT. In some example, the RT may be less mutagenic than a counterpart wildtype RT. In one embodiment, the RT herein is not mutagenic. Exemplary the reverse transcriptase include, but are not limited to, Human immunodeficiency virus (HIV) RT, Avian myeloblastosis virus (AMV) RT, Moloney murine leukemia virus (M-MLV) RT a group II intron RT, a group II intron-like RT, or a chimeric RT. In some embodiments, the functional domain comprises modified forms of these RTs, such as, engineered variants of Avian myeloblastosis virus (AMV) RT, Moloney murine leukemia virus (M-MLV) RT, or Human immunodeficiency virus (HIV) RT (see, e.g., Anzalone, et al., Search-and-replace genome editing without double-strand breaks or donor DNA, Nature. 2019 December; 576 (7785): 149-157). Without wishing to be bound by any particular theory, fusion proteins comprising a reverse transcriptase domain are useful inter alia for targeted editing, referred to herein as “prime editing,” of nucleic acid sequences in vitro and in vivo.

Also provided herein is a complex comprising a fusion protein described herein bound to a guide nucleic acid (e.g., gRNA or ωRNA). In another aspect, provided herein is a complex comprising a ωRNA described herein bound to a transposon family encoded nuclease or a homolog or ortholog thereof. For example, the complex comprises ωRNA described herein bound to a IS200/IS605 transposon family encoded nuclease (such as IscB, Isrb, and TnpB) or a homolog or ortholog thereof. Complexes comprising a guide nucleic acid (e.g., gRNA or ωRNA) bound, e.g., to protein effector polypeptide or a fusion protein described herein, or to a transposon family encoded nuclease or a homolog or ortholog thereof are also referred to as engineered, non-natural DNA and RNA targeting complexes, DNA and RNA targeting systems, and DNA and RNA targeting compositions herein.

In some embodiments, a DNA and RNA targeting complex described herein produces fewer indels in a target sequence that does not comprise the canonical TAM (5′-ATAAA) at is 3′-end as compared to the number of indels produced by a complex comprising a wild-type IscB and wild-type gRNA. For example, a DNA and RNA targeting complex described herein produces at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold fewer indels in a target sequence that does not comprise the canonical TAM (5′-ATAAA) at is 3′-end as compared to the number of indels produced by a complex comprising a wild-type IscB and wild-type gRNA. In some embodiments, indels can be measured using high-throughput sequencing.

In some embodiments, a DNA and RNA targeting complex described herein, where the protein effector polypeptide comprises a deaminase domain, exhibits an increased deamination efficacy in a target sequence that does not comprise the canonical TAM (5′-ATAAA) at is 3′-end as compared to the deamination activity of a complex comprising a wild-type IscB and wild-type gRNA. For example, the deamination efficiency of a DNA and RNA targeting complex, where the protein effector polypeptide comprises a deaminase domain, in a target sequence having a 3′-end that is not directly adjacent to the sequence 5′-ATAAA is at least is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold higher as compared to the deamination efficiency of a complex comprising a wild-type IscB and wild-type gRNA. The deamination activity can be measured using a deamination assay, PCR, sequencing or any combination thereof.

In some embodiments, a DNA and RNA targeting complex described herein exhibits a lower or decreased off-target activity, i.e., modification of a non-target sequence, as compared to the off-target activity of a complex comprising a wild-type IscB and wild-type gRNA. For example, the off-target activity of a DNA and RNA targeting complex described herein, is at least at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold lower than the off-target activity of a complex comprising a wild-type IscB and wild-type gRNA.

### Guide Nucleic Acids (ωRNAs)

As used herein, a “guide nucleic acid”, “gNA” refers to a nucleic acid that facilitates the targeting of protein effector polypeptide described herein to a target nucleic acid. The guide nucleic acid are also referred to as RNA or guide RNA (“gRNA”) herein. A guide nucleic acid can be RNA, DNA or a mix of RNA/DNA.

The ability of a guide nucleic acid to direct sequence-specific binding of a protein effector polypeptide described herein a target nucleic acid sequence can be assessed by any suitable assay. For example, components sufficient to form a complex comprising a protein effector polypeptide and the guide nucleic acid to be tested, can be provided to a host cell having the corresponding target nucleic acid sequence, such as by transfection with vectors encoding the components of the complex, followed by an assessment of preferential targeting (e.g., cleavage) within the target nucleic acid sequence, such as by Surveyor assay described in WO2022087494, content of which is incorporated herein by reference. Similarly, cleavage of a target nucleic acid sequence (or a sequence in the vicinity thereof) can be evaluated in a test tube by providing the target nucleic acid sequence, and components sufficient to form a complex comprising a protein effector polypeptide and the guide nucleic acid to be tested, and comparing binding or rate of cleavage at or in the vicinity of the target sequence between the test and control guide nucleic acid reactions. Other assays are possible, and will occur to those skilled in the art.

Some exemplary gRNAs amenable to various aspects described herein are described, for example, in PCT publications WO2022087494, WO2020180699, WO2021226363, WO2022261292, WO2021119275, WO2019237069, WO2018107028, WO2019067872 and WO2019067992, content of each of which is incorporated herein by reference in their entireties.

### ωRNA

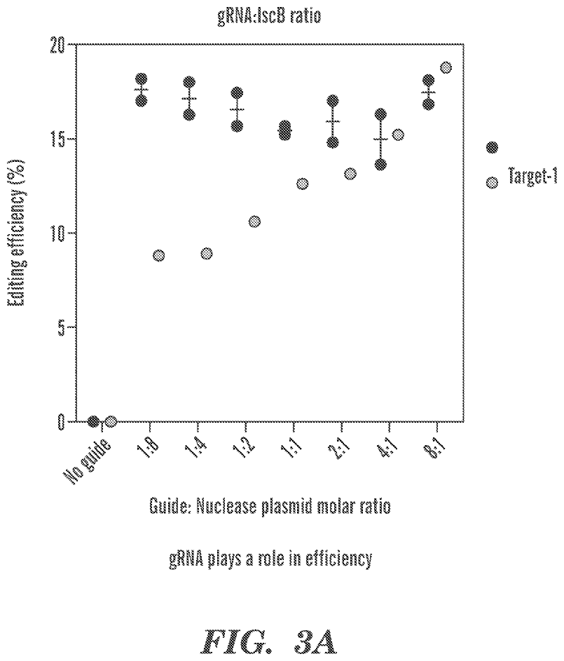

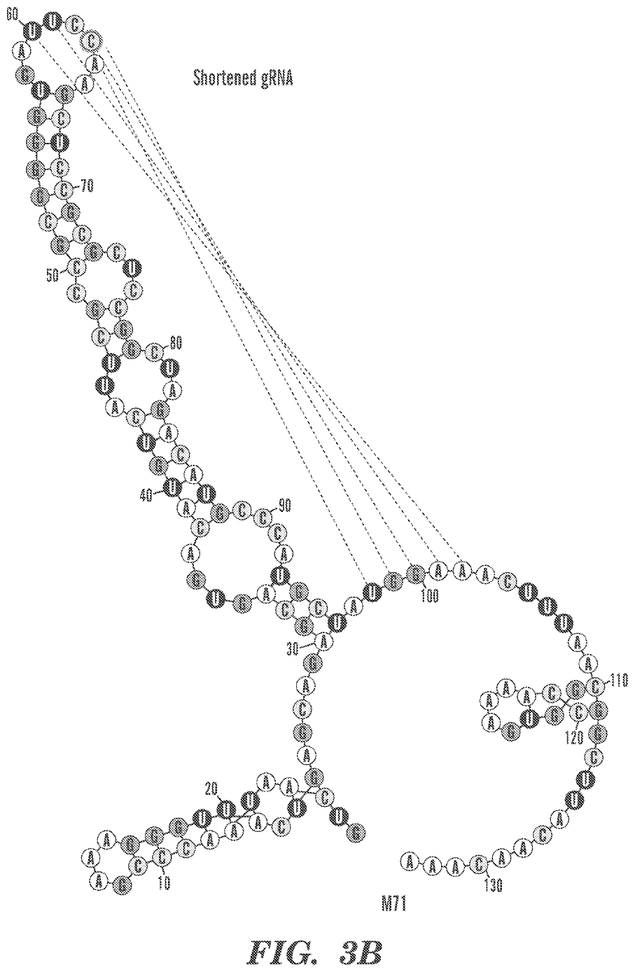

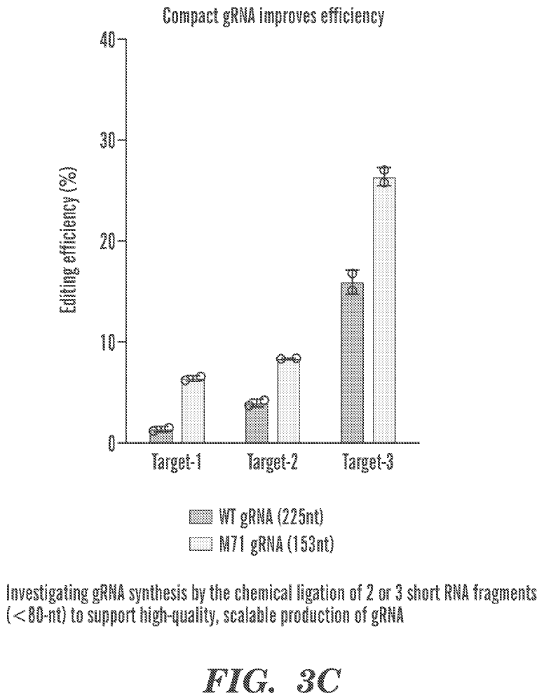

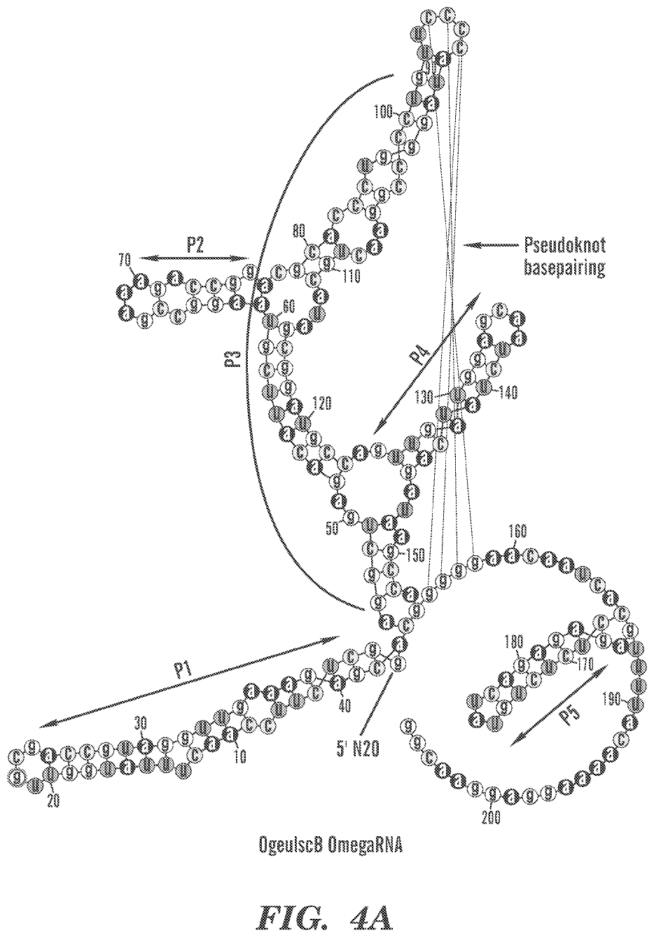

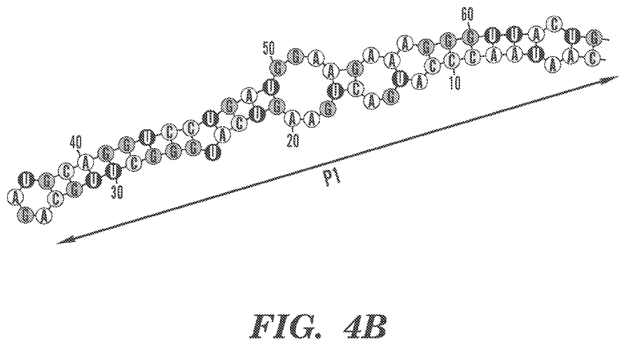

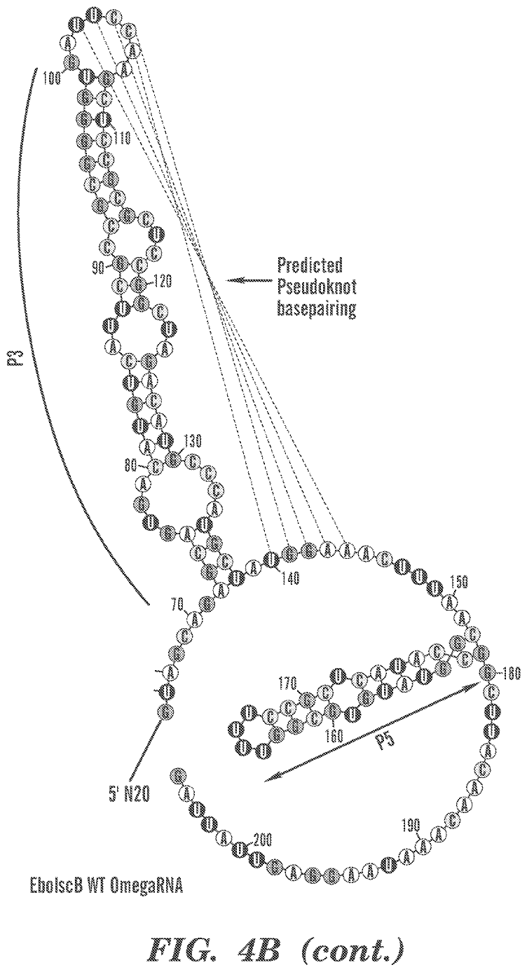

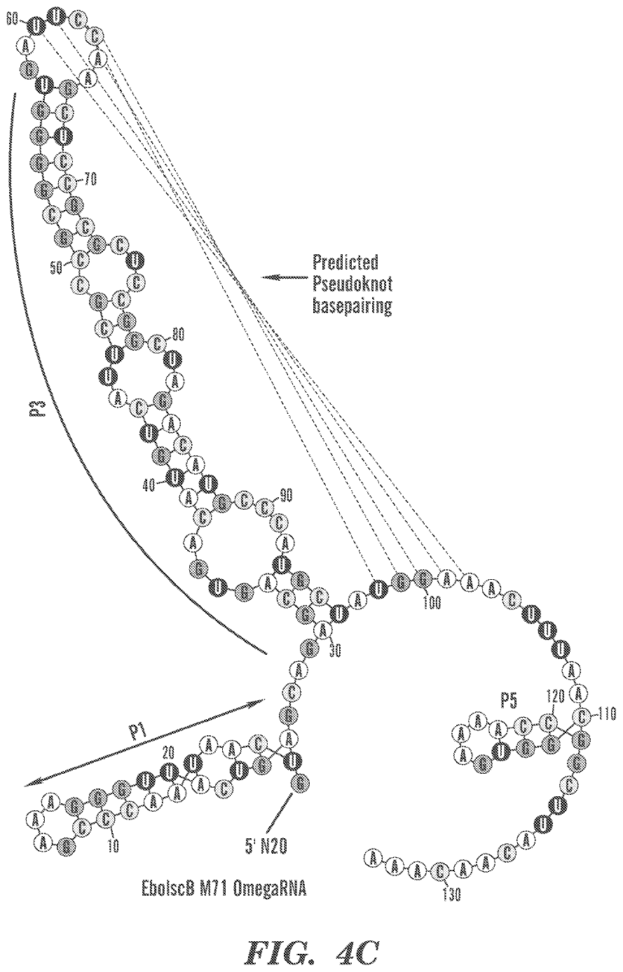

Generally, a ωRNA or guide RNA comprises a conserved region also referred to as a conserved portion. Without wishing the be bound a theory, the ωRNA interacts, e.g., associates or binds with a protein effector polypeptide via the conserved region. In some embodiments, the conserved region can comprise one or more hairpin or stem-loop structures. The hairpin closest to the 5′-end of the conserved region can be referred to as a 5′-stem-loop, 5′-hairpin or 5′-proximal hairpin. The hairpin closest to the 3′-end of the conserved region can be referred to as a 3′-stem-loop, 3′-hairpin or 3′-proximal hairpin. When the conserved region comprises three or more hairpins, the hairpins that are not near 5′- or 3′-end of the conserved region, (i.e., not a 5′-hairpin or not a 3′-hairpin) can be referred to as internal hairpins, internal stem-loop, non-end hairpins or non-end stem-loops herein. In some embodiments, the conserved region has a secondary structure comprising three hairpins. Without wishing to be bound by a theory, in some embodiments, the conserved region has a secondary structure comprising three hairpins as shown in FIGS. 3B and 4A-4C.

In some embodiments, a ωRNA described herein comprises a conserved portion of a ωRNA, e.g., a wild-type ωRNA (parent ωRNA), where the conserved region of the ωRNA comprises one or more (e.g., one, two, three, four, five, six, seven, eight, nine, ten or more) modifications as compared to the conserved region of the parent or wild-type ωRNA. In some embodiments, one or more nucleotides from the conserved region of the parent or wild-type ωRNA are not present in the ωRNA described herein.

### 5′-Hairpin

In various embodiments, the conserved region of ωRNA comprises a hairpin or stem-loop structure where at least a position of a hairpin or stem-loop structure (e.g., the first base-paired nucleotide at the 5′-end of the stem of the hairpin) is within 45 nucleotides, counting from the 5′-end of the conserved region. For example, the conserved region of the ωRNA comprises a hairpin or stem-loop structure the first base-paired nucleotide at the 5′-end of the stem of the hairpin is within 40, within 35, within 30, within 25, within 20, within 15, within 10 or within 5 nucleotides from the 5′-end of the conserved region. In some embodiments, the first base-pair nucleotide at the 5′-end of the stem of the hairpin is within 15, within 14, within 13, within 12, within 11, within 10, within 9, within 8, within 8, within 7, within 6, within 5, within 5, within 4, within 3, within 2 nucleotides from the 5′-end of the conserved region.

In some embodiments, the 5′-hairpin comprises a modification compared to the conserved region of the parent conserved region. For example, one or more nucleotides from the 5′-end hairpin of the parent or wild-type conserved region are removed or deleted. In some embodiments, 3 or more contiguous or non-contiguous nucleotides from the 5′-end hairpin of the parent or wild-type conserved region are removed or deleted in the conserved region of a ωRNA described herein. For example, from about 8 to about 50 contiguous or non-contiguous nucleotides from the 5′-end hairpin of the parent or wild-type conserved region are removed or deleted in the conserved region of a ωRNA described herein. In some embodiments, a ωRNA described herein comprises a conserved region where 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 32, 33, 34, 35, 36, 37, 38, 39, 40, 41, 42, 43, 44, 45, 46, 47, 48, 49 or 50 contiguous or non-contiguous nucleotides are removed or deleted from the 5′-hairpin compared to the wild-type or parent ωRNA. For example, a ωRNA described herein comprises a conserved region where 8, 12, 16, 20, 23, 27, 28, 31, 35, 39, 42, or 46 contiguous or non-contiguous nucleotides are removed or deleted from the 5′-hairpin compared to the wild-type or parent ωRNA. In some embodiments, the ωRNA comprises a conserved region where 8, 20, 35, 42, or 46 contiguous or non-contiguous nucleotides are removed or deleted from the 5′-hairpin compared to the wild-type or parent ωRNA.

In some embodiments, a ωRNA described herein comprises a conserved region where 3, 7, 11, 15, 18, 22, 26, 30, 34, 37 or 41 contiguous or non-contiguous nucleotides are removed or deleted from the 5′-hairpin compared to the wild-type or parent ωRNA. For example, the ωRNA comprises a conserved region where 3, 15, 30, 37, or 41 contiguous or non-contiguous nucleotides are removed or deleted from the 5′-hairpin compared to the wild-type or parent ωRNA.

In some embodiments, nucleotides at position 12-57, 14-55, 16-54, 18-52, 20-50, 22-48, 24-46, 26-45, 28-43, 30-41 or 32-39 of the wild-type or parent conserved region are removed or deleted (counting from 5′-end of the conserved region of the parent ωRNA). For example, nucleotides at position 12-57, 14-55, 18-52, 24-46, or 26-45 of the wild-type or parent conserved region are removed or deleted (counting from 5′-end of the conserved region of the parent ωRNA). In some embodiments, nucleotides at position 11-56, 13-54, 15-53, 17-51, 19-49, 21-47, 23-45, 25-44, 27-42, 29-41 or 32-39 of the wild-type or parent conserved region are removed or deleted (counting from first base-paired nucleotide at 5′-end of the 5′-hairpin). For example, nucleotides at position 11-56, 13-54, 17-51, 23-45, or 25-44 of the wild-type or parent conserved region are removed or deleted (counting from first base-paired nucleotide at 5′-end of the 5′-hairpin).

It is noted that the nucleotides removed or deleted from the conserved region of the wild-type or parent ωRNA's 5′-hairpin can be replaced with a linker. For example, the deleted or the removed nucleotides can be replaced with a nucleotide or a non-nucleotide linker. In some embodiments, the nucleotides removed or deleted from the conserved region of the wild-type or parent ωRNA's 5′-hairpin are replaced with a nucleotide sequence, e.g., a single-stranded nucleic acid comprising from about comprising from about 4 nucleotides to about 15 nucleotides. For example, the deleted or the removed nucleotides can be replaced with nucleotide linker comprising 4, 5, 6, 7, 8, 9, or 10 nucleotides. In some embodiments, the deleted or the removed nucleotides can be replaced with a nucleotide sequence comprising 4, 5 or 6 nucleotides. In some embodiments, the deleted or the removed nucleotides are replaced with the nucleotide sequence GAAA or GAAAA. For example, the deleted or the removed nucleotides are replaced with the nucleotide sequence GAAAA

The 5′-hairpin comprises a double-stranded region (i.e., stem portion) and a single-stranded region (i.e., a pin-loop) connecting the two strands forming the stem portion of the hairpin. The stem portion of the 5′-hairpin can comprise a double-stranded region comprising 4 or more nucleotide base pairs. For example, the stem portion of the 5′-hairpin can comprise a double-stranded region comprising from about 5 to about 30 nucleotide base-pairs. In some embodiments, the stem portion of the 5′-hairpin comprises a double-stranded region comprising from about 8 to about 25 nucleotide base-pairs. For example, the stem portion of the 5′-hairpin comprises a double-stranded region comprising about 9 nucleotide base-pairs, about 10 nucleotide base-pairs, about 11 nucleotide base-pairs, about 12 nucleotide base-pairs, about 13 nucleotide base-pairs, about 14 nucleotide base-pairs, about 15 base pairs nucleotide base-pairs, about 16 base pairs nucleotide base-pairs, about 17 base pairs nucleotide base-pairs, about 18 base pairs nucleotide base-pairs, about 19 base pairs nucleotide base-pairs, or about 20 base pairs nucleotide base-pairs. In some embodiments, the stem portion of the 5′-hairpin comprises a double-stranded region comprising 8, 9, 10, 11 or 12 nucleotide base pairs. In some preferred embodiments, the stem portion of the 5′-hairpin comprises a double-stranded region comprising 9, 10 or 11 nucleotide base pairs. For example, the stem portion of the 5′-hairpin comprises a double-stranded region comprising 10 nucleotide base pairs.

In some embodiments, the stem portion of the 5′-hairpin comprises a double-stranded region comprising 1, 2, 3, 4, 5 6 or 7 nucleotide base-pairs.

In some embodiments, the stem portion of the 5′-hairpin comprises a mismatch. For example, the stem portion of the 5′-hairpin comprises a mismatch (e.g., an A: C mismatch) at position 2, 3, 4, 5, 6, 7, 8, or 9, counting from the first base-paired nucleotide at the 5′-end of the 5′-hairpin.

In some embodiments, the stem portion of the 5′-hairpin comprises a double-stranded region comprising 8, 9, 10, 11, or 12 nucleotide base-pairs and the stem portion further comprises a mismatch in the stem portion. For example, the stem portion of the 5′-hairpin comprises a double-stranded region comprising 8, 9, 10, 11, or 12 nucleotide base-pairs and the stem portion further comprises a mismatch (e.g., an A: C mismatch) at position 3, 4, 5, 6, 7, or 8, counting from the first base-paired nucleotide at the 5′-end of the 5′-hairpin. In some embodiments, the mismatch (e.g., an A: C mismatch) is at position 3, 4, 5, 6 or 7. For example, the mismatch (e.g., an A: C mismatch) is at position 4 or 5, counting from the first base-paired nucleotide at the 5′-end of the 5′-hairpin.

The pin-loop portion of the 5′-hairpin can be 3 nucleotides or longer. For example, the pin-loop portion of the 5′-hairpin can be from about 4 nucleotides to about 15 nucleotides. For example, the pin-loop portion of the 5′-hairpin can be 4, 5, 6, 7, 8, 9, or 10 nucleotides in length. In some embodiments, the pin-loop portion of the 5′-hairpin is 4, 5 or 6 nucleotides in length.

A total length of the 5′-hairpin, e.g., from the first base-paired nucleotide at the 5′-end to the last bas-paired nucleotide at the 3′-end of the hairpin, can be from about from about 20 nucleotides to about 65 nucleotides. For example, the 5′-hairpin can comprise from about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, or about 45 nucleotides. In some embodiments, the 5′-hairpin comprises 23, 24, 25, 26 or 27 nucleotides. For example, the 5′-hairpin comprises, 24, 25 or 26, e.g., 25 nucleotides.

The 5′-hairpin can comprise a nucleic acid modification, e.g., to stabilize the hairpin. For example, the 5′-hairpin can comprise a nucleic acid modification selected from the group consisting of nucleobase modifications, sugar modifications, inter-sugar linkage modifications (modified internucleoside linkages), conjugates (e.g., ligands), and any combinations thereof. The modification can be present at any location in the 5′-hairpin. In some embodiments, the stem portion of the 5′-hairpin comprises a nucleic acid modification. In some embodiments, the pin-loop portion of the 5′-hairpin comprises a nucleic acid modification. In yet some other embodiments, both the stem portion and the pin-loop portion comprise independently selected nucleic acid modification.

In some embodiments, the stem portion and/or the pin-loop portion of the 5′-hairpin comprises a modified nucleotide selected independently from the group consisting of 2′-O-methyl(2′-OMe) nucleotides, 2′-fluoro (2′-F) nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl(2′-MOE) nucleotides, 2′-O-allyl nucleotides, 2′-C-allyl ribose nucleotides, 2′-O-N-methylacetamido (2′-O-NMA) nucleotides, a 2′-O-dimethylaminoethoxyethyl(2′-O-DMAEOE) nucleotides, 2′-O-aminopropyl(2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a non-natural or modified nucleobase; or a combination thereof. For example, the stem portion and/or the pin-loop portion of the 5′-hairpin comprises a 2′-O-methyl modified nucleotide. In another example, the stem portion and/or the pin-loop portion of the 5′-hairpin comprises a 2′-fluoro modified nucleotide.

The 5′-hairpin can also comprise one or more (e.g., 1, 2, 3, 4, 5 or more) non-natural or modified nucleobases. The non-natural or modified nucleobase can be present in the stem portion or the pin-loop portion of the 5′-hairpin. In some embodiments, the stem portion of the 5′-hairpin comprises one or more (e.g., 1, 2, 3, 4, 5 or more) non-natural or modified nucleobases. In another embodiment, the pin-loop portion of the 5′-hairpin comprises one or more (e.g., 1, 2, 3, 4, 5 or more) non-natural or modified nucleobases. In yet some other embodiments, both the stem portion and the pin-loop portion of the 5′-hairpin comprises one or more (e.g., 1, 2, 3, 4, 5 or more) independently selected non-natural or modified nucleobases.

The 5′-hairpin can also comprise at least one (e.g., 1, 2, 3, 4, 5, or more) modified internucleoside linkages. Exemplary modified internucleoside linkages are described herein and include, but are not limited to, phosphorothioate and MMI linkages. Like other modifications, a modified internucleotide linkage can be present in the stem portion and/or the pin-loop portion of the 5′-hairpin.

In some embodiments, the stem portion of the 5′-hairpin comprises one or more (e.g., 1, 2, 3, 4, 5 or more) modified internucleoside linkages (e.g., phosphorothioate and/or MMI linkages). In another embodiment, the pin-loop portion of the 5′-hairpin comprises one or more (e.g., 1, 2, 3, 4, 5 or more) modified internucleoside linkages (e.g., phosphorothioate and/or MMI linkages). In yet some other embodiments, both the stem portion and the pin-loop portion of the 5′-hairpin comprises one or more (e.g., 1, 2, 3, 4, 5 or more) independently selected modified internucleoside linkages (e.g., phosphorothioate and/or MMI linkages). In some embodiments, the 5′-hairpin comprises a modified internucleoside linkage (e.g., phosphorothioate and/or MMI linkage) between the stem portion and the pin-loop portion of the 5′-hairpin.

Without wishing to be bound by a theory, increasing the stability, e.g., stability of the stem of the 5′-hairpin can provide increased or higher nucleic acid modification, e.g., editing activity and/or efficiency. Accordingly, in some embodiments, the stem portion of the 5′-hairpin comprises a duplex stabilizing modification. Some exemplary duplex stabilizing modifications include, but are not limited to, 2′-F nucleotides, 2′-OMe nucleotides, 2′-methoxyethyl nucleotides, 2,6-diaminopurine nucleotides, 5-methyl cytidine, N4-ethyl cytidine, 5-propynyl cytidine, 5-propynyl uridine, 5-hydroxybutynl-2′-deoxyuridine, 8-aza-7-deazaguanosine, a locked nucleic acid (LNA), and/or covalent cross-linking of two strands of the duplex. In some embodiments, the stem portion comprises a covalent linkage between two nucleotides, optionally the covalent linkage is at a position other than 5′ and 3′ of the nucleotides.

In some embodiments, a duplex stabilizing modification comprises a A:U base pair in the stem of the 5′-hairpin to a G: C base-pair. For example, one or more, e.g., 1, 2, 3, 4, 5, 6, 7 or more A:U base pairs in the stem of the 5′-hairpin ton G: C base-pairs. In some embodiments, a duplex stabilizing modification comprises replacing a A:U base pair in the stem of the 5′-hairpin with a G: C base-pair. For example, one or more, e.g., 1, 2, 3, 4, 5, 6, 7 or more A:U base pairs in the stem of the 5′-hairpin ton G: C base-pairs. In some embodiments, a duplex stabilizing modification comprises replacing mismatch in the stem of the 5′-hairpin with a G: C or A:U base-pair.

It is known that guanine tetrads are formed by sequences rich in guanine, such as GGGGC. Thus, in order to reduce formation of guanine tetrads, G and C in the G: C base-pair replacing a A:U base-pair or a mismatch are positioned so that there are no more than 5, preferably no more than 4, and more preferably no more than 3 consecutive G in the sequence of the stem.

In some embodiments, an A: C mismatch in the stem of the 5′-hairpin of a wild-type or parent ωRNA conserved region is replaced with a G: C base-pair. For example, the A in the mismatch is replaced by G, or the A is replaced by C and the Cis replaced by G. In some other embodiments, the A: C mismatch in the stem of the 5′-hairpin is replaced by an A:U base-pair. For example, the C in the mismatch is replaced by U, or the A is replaced by U and the Cis replaced by A.

In some embodiments, one or more (e.g., 1, 2, 3, 4, 5 or more) A:U base-pairs present in the stem of the 5′-hairpin of the wild-type or parent RNA conserved region are deleted.

In some embodiments, one or more nucleotides present at the 5′-end of the wild-type or parent ωRNA conserved region, that are not involved in forming the 5′-hairpin are deleted.

### 3′-Hairpin

In various embodiments, the conserved region of ωRNA comprises a hairpin or stem-loop structure where at least a position of a hairpin or stem-loop structure (e.g., the first base-paired nucleotide at the 3′-end of the stem of the hairpin) is within 50 nucleotides, counting from the 3′-end of the conserved region. For example, the conserved region of the ωRNA comprises a hairpin or stem-loop structure the first base-paired nucleotide at the 3′-end of the stem of the hairpin is within 45, within 40, within 35, within 30, within 25, within 20, within 15, within 10 or within 5 nucleotides from the 3′-end of the conserved region. In some embodiments, the first base-pair nucleotide at the 3′-end of the stem of the 3′-hairpin is within 35, within 34, within 33, within 32, within 31, within 30, within 29, within 28, within 27, within 26, within 25, within 24, within 23, within 22, within 21, within 20, within 19, within 18, within 17, within 16, within 15, within 14, within 13, within 12, within 11, or within 10 nucleotides from the 3′-end of the conserved region. For example, the first base-pair nucleotide at the 3′-end of the stem of the 3′-hairpin is within 20, within 19, within 18, within 17, within 16, within 15, within 14, within 13, within 12, within 11, or within 10 nucleotides from the 3′-end of the conserved region. In some embodiments, the first base-pair nucleotide at the 3′-end of the stem of the 3′-hairpin is within 9, within 8, within 7, within 6, within 5, within 4, within 3, within 2 nucleotides from the 3′-end of the conserved region.

In some embodiments, the 3′-hairpin comprises a modification compared to the conserved region of the parent conserved region. For example, one or more nucleotides from the 3′-hairpin of the parent or wild-type conserved region are removed or deleted. In some embodiments, 1 or at least 2 or more contiguous or non-contiguous nucleotides from the 3′-end hairpin of the parent or wild-type conserved region are removed or deleted in the conserved region of a ωRNA described herein. For example, from 1 to about 25 contiguous or non-contiguous nucleotides from the 3′-end hairpin of the parent or wild-type conserved region are removed or deleted in the conserved region of a ωRNA described herein. In some embodiments, a ωRNA described herein comprises a conserved region where 1, 2, 3, 4, 5, 6, 7, 8, 9, 10, 11, 12, 13, 14, 15, 16, 17, 18, 19, 20, 21, 22, 23, 24, or 25 contiguous or non-contiguous nucleotides are removed or deleted from the ‘-hairpin compared to the wild-type or parent ωRNA. For example, a ωRNA described herein comprises a conserved region where 2, 6, 8, 10, 12, 16, or 20 contiguous or non-contiguous nucleotides are removed or deleted from the 3′-hairpin compared to the wild-type or parent ωRNA. In some embodiments, the ωRNA comprises a conserved region where 2, 10, 12, or 16 contiguous or non-contiguous nucleotides are removed or deleted from the 3′-hairpin compared to the wild-type or parent ωRNA.

In some embodiments, a ωRNA described herein comprises a conserved region where 6, 10, 12, 14, 16, 20 or 22 contiguous or non-contiguous nucleotides are removed or deleted from the 3′-hairpin compared to the wild-type or parent ωRNA. For example, the ωRNA comprises a conserved region where 6, 14, 16, or 20 contiguous or non-contiguous nucleotides are removed or deleted from the 3′-hairpin compared to the wild-type or parent ωRNA.

In some embodiments, nucleotides at position 159-172, 160-171, 161-170, 163-168, 158-173, 156-175 or 154-177 of the wild-type or parent conserved region are removed or deleted (counting from 5′-end of the conserved region of the parent ωRNA). For example, nucleotides at position 159-172, 163-168, 158-173, or 156-175 of the wild-type or parent conserved region are removed or deleted (counting from 5′-end of the conserved region of the parent ωRNA. In some embodiments, nucleotides at position 6-19, 7-18, 8-17, 10-15, 5-20, 3-22, or 1-24 of the wild-type or parent conserved region are removed or deleted (counting from first base-paired nucleotide at 5′-end of the 3′-hairpin). For example, nucleotides at position 6-19, 10-15, 5-20, or 3-22 of the wild-type or parent conserved region are removed or deleted (counting from first base-paired nucleotide at 5′-end of the 3′-hairpin).

It is noted that the nucleotides removed or deleted from the conserved region of the wild-type or parent ωRNA's 3′-hairpin can be replaced with a linker. For example, the deleted or the removed nucleotides can be replaced with a nucleotide or a non-nucleotide linker. In some embodiments, the nucleotides removed or deleted from the conserved region of the wild-type or parent ωRNA's 3′-hairpin are replaced with a nucleotide sequence, e.g., a single-stranded nucleic acid comprising from about comprising from about 4 nucleotides to about 15 nucleotides. For example, the deleted or the removed nucleotides can be replaced with nucleotide linker comprising 4, 5, 6, 7, 8, 9, or 10 nucleotides. In some embodiments, the deleted or the removed nucleotides can be replaced with a nucleotide sequence comprising 4, 5 or 6 nucleotides. In some embodiments, the deleted or the removed nucleotides are replaced with the nucleotide sequence GAAA or GAAAA. For example, the deleted or the removed nucleotides are replaced with the nucleotide sequence GAAA

The 3′-hairpin comprises a double-stranded region (i.e., stem portion) and a single-stranded region (i.e., pin-loop portion) connecting the two strands forming the stem portion of the hairpin. The stem portion of the 3′-hairpin can comprise a double-stranded region comprising 4 or more nucleotide base pairs. For example, the stem portion of the 3′-hairpin can comprise a double-stranded region comprising from about 3 to about 20 nucleotide base-pairs. In some embodiments, the stem portion of the 3′-hairpin comprises a double-stranded region comprising from about 4 to about 15 nucleotide base-pairs. For example, the stem portion of the 3′-hairpin comprises a double-stranded region comprising about 5, about 6, about 7, about 8, about 9 nucleotide base-pairs, about 10 nucleotide base-pairs, about 11 nucleotide base-pairs, about 12 nucleotide base-pairs, about 13 nucleotide base-pairs, about 14 nucleotide base-pairs, or about 15 nucleotide base-pairs. In some embodiments, the stem portion of the 3′-hairpin comprises a double-stranded region comprising 3, 4, 5, 6, 7, 8, 9, or 10 nucleotide base pairs. In some preferred embodiments, the stem portion of the 3′-hairpin comprises a double-stranded region comprising 3, 4, 5, or 6 nucleotide base pairs. For example, the stem portion of the 3′-hairpin comprises a double-stranded region comprising 3 or 4 nucleotide base pairs.

The pin-loop portion of the 3′-hairpin can be 3 nucleotides or longer. For example, the pin-loop portion of the 3′-hairpin can be from about 4 nucleotides to about 15 nucleotides. For example, the pin-loop portion of the 3′-hairpin can be 4, 5, 6, 7, 8, 9, or 10 nucleotides in length. In some embodiments, the pin-loop portion of the 3′-hairpin is 4, 5 or 6 nucleotides in length.

A total length of the 3′-hairpin, e.g., from the first base-paired nucleotide at the 5′-end to the last base-paired nucleotide at the 3′-end of the hairpin, can be from about 8 nucleotides to about 25 nucleotides.

For example, the 3′-hairpin can comprise about 9, about 10, about 11, about 12, about 13, about 14, about 15, about 16, about 17, about 18, about 19, or about 20 nucleotides. In some embodiments, the 3′-hairpin comprises 23, 24, 25, 26 or 27 nucleotides. For example, the 3′-hairpin comprises 11, 12, 13, 14 or 15 nucleotides.

The 3′-hairpin can comprise a nucleic acid modification, e.g., to stabilize the hairpin. For example, the 3′-hairpin can comprise a nucleic acid modification selected from the group consisting of nucleobase modifications, sugar modifications, inter-sugar linkage modifications (modified internucleoside linkages), conjugates (e.g., ligands), and any combinations thereof. The modification can be present at any location in the 3′-hairpin. In some embodiments, the stem portion of the 3′-hairpin comprises a nucleic acid modification. In some embodiments, the pin-loop portion of the 3′-hairpin comprises a nucleic acid modification. In yet some other embodiments, both the stem portion and the pin-loop portion comprise independently selected nucleic acid modification.

In some embodiments, the stem portion and/or the pin-loop portion of the 3′-hairpin comprises a modified nucleotide selected independently from the group consisting of 2′-O-methyl(2′-OMe) nucleotides, 2′-fluoro (2′-F) nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl(2′-MOE) nucleotides, 2′-O-allyl nucleotides, 2′-C-allyl ribose (2′-O-NMA) nucleotides, 2′-O-N-methylacetamido nucleotides, a 2′-O-dimethylaminoethoxyethyl(2′-O-DMAEOE) nucleotides, 2′-O-aminopropyl(2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a non-natural or modified nucleobase; or a combination thereof. For example, the stem portion and/or the pin-loop portion of the 3′-hairpin comprises a 2′-O-methyl modified nucleotide. In another example, the stem portion and/or the pin-loop portion of the 3′-hairpin comprises a 2′-fluoro modified nucleotide.

The 3′-hairpin can also comprise one or more (e.g., 1, 2, 3, 4, 5 or more) non-natural or modified nucleobases. The non-natural or modified nucleobase can be present in the stem portion or the pin-loop portion of the 3′-hairpin. In some embodiments, the stem portion of the 3′-hairpin comprises one or more (e.g., 1, 2, 3, 4, 5 or more) non-natural or modified nucleobases. In another embodiment, the pin-loop portion of the 3′-hairpin comprises one or more (e.g., 1, 2, 3, 4, 5 or more) non-natural or modified nucleobases. In yet some other embodiments, both the stem portion and the pin-loop portion of the 3′-hairpin comprises one or more (e.g., 1, 2, 3, 4, 5 or more) independently selected non-natural or modified nucleobases.

The 3′-hairpin can also comprise at least one (e.g., 1, 2, 3, 4, 5, or more) modified internucleoside linkages. Exemplary modified internucleoside linkages are described herein and include, but are not limited to, phosphorothioate and MMI linkages. Like other modifications, a modified internucleotide linkage can be present in the stem portion and/or the pin-loop portion of the 3′-hairpin.

In some embodiments, the stem portion of the 3′-hairpin comprises one or more (e.g., 1, 2, 3, 4, 5 or more) modified internucleoside linkages (e.g., phosphorothioate and/or MMI linkages). In another embodiment, the pin-loop portion of the 3′-hairpin comprises one or more (e.g., 1, 2, 3, 4, 5 or more) modified internucleoside linkages (e.g., phosphorothioate and/or MMI linkages). In yet some other embodiments, both the stem portion and the pin-loop portion of the 3′-hairpin comprises one or more (e.g., 1, 2, 3, 4, 5 or more) independently selected modified internucleoside linkages (e.g., phosphorothioate and/or MMI linkages). In some embodiments, the 3′-hairpin comprises a modified internucleoside linkage (e.g., phosphorothioate and/or MMI linkage) between the stem portion and the pin-loop portion of the 3′-hairpin.

Without wishing to be bound by a theory, increasing the stability, e.g., stability of the stem of the 3′-hairpin can provide increased or higher nucleic acid modification, e.g., editing activity and/or efficiency. Accordingly, in some embodiments, the stem portion of the 3′-hairpin comprises a duplex stabilizing modification. Some exemplary duplex stabilizing modifications include, but are not limited to, 2′-F nucleotides, 2′-OMe nucleotides, 2′-methoxyethyl nucleotides, 2,6-diaminopurine nucleotides, 5-methyl cytidine, N4-ethyl cytidine, 5-propynyl cytidine, 5-propynyl uridine, 5-hydroxybutynl-2′-deoxyuridine, 8-aza-7-deazaguanosine, a locked nucleic acid (LNA), and/or covalent cross-linking of two strands of the duplex. In some embodiments, the stem portion comprises a covalent linkage between two nucleotides, optionally the covalent linkage is at a position other than 5′ and 3′ of the nucleotides.

In some embodiments, a duplex stabilizing modification comprises changing an A:U base pair in the stem of the 3′-hairpin to a G: C base-pair. For example, one or more, e.g., 1, 2, 3, 4, 5 or more A:U base pairs in the stem of the 3′-hairpin ton G: C base-pairs. It is known that guanine tetrads are formed by sequences rich in guanine, such as GGGGC. Thus, in order to reduce formation of guanine tetrads, G and C in the G: C base-pair replacing a A:U base-pair are positioned so that there are no more than 5, preferably no more than 4, and more preferably no more than 3 consecutive the sequence of the stem.

In some embodiments, a duplex stabilizing modification comprises a A:U base pair in the stem of the 3′-hairpin to a G: C base-pair. For example, one or more, e.g., 1, 2, 3, 4, 5, 6, 7 or more A:U base pairs in the stem of the 3′-hairpin ton G: C base-pairs. In some embodiments, a duplex stabilizing modification comprises replacing a A:U base pair in the stem of the 5′-hairpin with a G: C base-pair. For example, one or more, e.g., 1, 2, 3, 4, 5, 6, 7 or more A:U base pairs in the stem of the 3′-hairpin ton G: C base-pairs. In some embodiments, a duplex stabilizing modification comprises replacing mismatch in the stem of the 3′-hairpin with a G: C or A:U base-pair. It is known that guanine tetrads are formed by sequences rich in guanine, such as GGGGC. Thus, in order to reduce formation of guanine tetrads, G and C in the G: C base-pair replacing a A:U base-pair or a mismatch are positioned so that there are no more than 5, preferably no more than 4, and more preferably no more than 3 consecutive G in the sequence of the stem.

In some embodiments, an A: C mismatch in the stem of the 3′-hairpin of a wild-type or parent ωRNA conserved region is replaced with a G: C base-pair. For example, the A in the mismatch is replaced by G, or the A is replaced by C and the Cis replaced by G. In some other embodiments, the A: C mismatch in the stem of the 3′-hairpin is replaced by an A:U base-pair. For example, the C in the mismatch is replaced by U, or the A is replaced by U and the Cis replaced by A.

In some embodiments, one or more (e.g., 1, 2, 3, 4, 5 or more) A:U base-pairs present in the stem of the 3′-hairpin of the wild-type or parent ωRNA conserved region are deleted.

### Middle Hairpin

In various embodiments, the conserved region of ωRNA comprises, at least three hairpin or stem-loop structures, where at least one of the internal hairpins (i.e., internal-hairpin) comprises a modification compared to the conserved region of the parent conserved region. For example, one or more nucleotides from the internal-hairpin of the parent or wild-type conserved region are removed or deleted. In some embodiments, 1 or at least 2 or more contiguous or non-contiguous nucleotides from the 3′-end hairpin of the parent or wild-type conserved region are removed or deleted in the conserved region of a ωRNA described herein. For example, from 1 to about 25 contiguous or non-contiguous nucleotides from the 3′-end hairpin of the parent or wild-type conserved region are removed or deleted in the conserved region of a ωRNA described herein. In some embodiments, a ωRNA described herein comprises a conserved region where 1, 2, 3, 4, 5, 6, 7, 8, 9, 10, 11, 12, 13, 14, 15, 16, 17, 18, 19, 20, 21, 22, 23, 24, or 25 contiguous or non-contiguous nucleotides are removed or deleted from the ′-hairpin compared to the wild-type or parent ωRNA.

It is noted that the nucleotides removed or deleted from the conserved region of the wild-type or parent ωRNA's internal-hairpin can be replaced with a linker. For example, the deleted or the removed nucleotides can be replaced with a nucleotide or a non-nucleotide linker. In some embodiments, the nucleotides removed or deleted from the conserved region of the wild-type or parent ωRNA's internal-hairpin can be replaced with a linker are replaced with a nucleotide sequence, e.g., a single-stranded nucleic acid comprising from about comprising from about 4 nucleotides to about 15 nucleotides. For example, the deleted or the removed nucleotides can be replaced with nucleotide linker comprising 4, 5, 6, 7, 8, 9, or 10 nucleotides. In some embodiments, the deleted or the removed nucleotides can be replaced with a nucleotide sequence comprising 4, 5 or 6 nucleotides. In some embodiments, the deleted or the removed nucleotides are replaced with the nucleotide sequence GAAA or GAAAA. For example, the deleted or the removed nucleotides are replaced with the nucleotide sequence GAAA

The internal-hairpin comprises a double-stranded region (i.e., stem portion) and a single-stranded region (i.e., loop portion) connecting the two strands forming the stem portion of the hairpin. The stem portion of the internal-hairpin can comprise a double-stranded region comprising 4 or more nucleotide base pairs. For example, the stem portion of the internal-hairpin can comprise a double-stranded region comprising from about 3 to about 20 nucleotide base-pairs. In some embodiments, the stem portion of the internal-hairpin comprises a double-stranded region comprising from about 10 to about 30 nucleotide base-pairs. For example, the stem portion of the internal-hairpin comprises a double-stranded region comprising about 11 nucleotide base-pairs, about 12 nucleotide base-pairs, about 13 nucleotide base-pairs, about 14 nucleotide base-pairs, about 15 nucleotide base-pairs, about 16 nucleotide base-pairs, about 17 nucleotide base-pairs, about 18 nucleotide base-pairs, about 19 nucleotide base-pairs, about 20 nucleotide base-pairs, about 21 nucleotide base-pairs, about 22 nucleotide base-pairs, about 23 nucleotide base-pairs, about 24 nucleotide base-pairs, about 25 nucleotide base-pairs, about 26 nucleotide base-pairs, about 27 nucleotide base-pairs, about 28 nucleotide base-pairs, or about 29 nucleotide base-pairs.

In some embodiments, the stem portion of the internal-hairpin comprises a double-stranded region comprising 17, 18, 19, 20, 21, 22, 23, 24, or 25 nucleotide base pairs. In some preferred embodiments, the stem portion of the internal-hairpin comprises a double-stranded region comprising 18, 19, 20, 21, 22 or 23 nucleotide base pairs. For example, the stem portion of the internal-hairpin comprises a double-stranded region comprising 19 nucleotide base pairs.

In some embodiments, the stem portion of the internal-hairpin comprises two or more contiguous mismatches in the duplex region, i.e., the stem comprises one or more internal loops. For example, the stem portion of the internal-hairpin comprises two, three or four internal loops.

In some embodiments, the stem portion of the internal-hairpin comprises a double-stranded region comprising 15, 16, 17, 18, 19, 21, 22, 23, 24 or 25 nucleotide base-pairs and the stem portion further comprises 2, 3, or 4 internal loops. For example, the stem portion of the internal-hairpin comprises a double-stranded region comprising 18, 19, 20, 21, or 22 nucleotide base-pairs and the stem portion further comprises further comprises 2, 3, or 4 internal loops. In some embodiments, the stem portion of the internal-hairpin comprises a double-stranded region comprising 18, 19, 20, 21, or 22 nucleotide base-pairs and the stem portion further comprises at least one internal loop at least one (e.g., one, two, or all three) of positions 4, 14 and 17.

A pin-loop portion of the internal-hairpin can be 3 nucleotides or longer. For example, the pin-loop portion of the internal-hairpin can be from about 5 nucleotides to about 25 nucleotides. For example, the pin-loop portion of the internal-hairpin can be 5, 6, 7, 8, 9, 10, 11, 12, 13, 14, 15, 16, 17, 18, 19, or 20 nucleotides in length. In some embodiments, the pin-loop portion of the internal-hairpin is 5, 6, 7, 8, 9 or 10 nucleotides in length.

A total length of the internal-hairpin, e.g., from the first base-paired nucleotide at the 5′-end to the last base-paired nucleotide at the 3′-end of the hairpin, can be from about 55 nucleotides to about 85 nucleotides. For example, the internal-hairpin can comprise about 60, about 61, about 62, about 63, about 64, about 65, about 66, about 67, about 68, about 69, or about 70, about 71, about 72, about 72, about 73, about 75, about 76, about 77, about 78, about 79 or about 80 nucleotides. In some embodiments, the internal-hairpin comprises 65, 66, 67, 68, 69, 70, 71, 72, 73, 74, 75, or 76 nucleotides. For example, the internal-hairpin comprises 66, 67, 68, 69, 70, 71, 72, 73, 74 or 75 nucleotides. In some embodiments, the internal-hairpin comprises about 66 nucleotides.

The internal-hairpin can comprise a nucleic acid modification, e.g., to stabilize the hairpin. For example, the internal-hairpin can comprise a nucleic acid modification selected from the group consisting of nucleobase modifications, sugar modifications, inter-sugar linkage modifications (modified internucleoside linkages), conjugates (e.g., ligands), and any combinations thereof. The modification can be present at any location in the internal-hairpin. In some embodiments, the stem portion of the internal-hairpin comprises a nucleic acid modification. In some embodiments, the loop portion of the internal-hairpin comprises a nucleic acid modification. In yet some other embodiments, both the stem portion and the loop portion comprise independently selected nucleic acid modification.

In some embodiments, the stem portion and/or the loop portion of the internal-hairpin comprises a modified nucleotide selected independently from the group consisting of 2′-O-methyl(2′-OMe) nucleotides, 2′-fluoro (2′-F) nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl(2′-MOE) nucleotides, 2′-O-allyl nucleotides, 2′-C-allyl ribose (2′-O-NMA) nucleotides, 2′-O-N-methylacetamido nucleotides, a 2′-O-dimethylaminoethoxyethyl(2′-O-DMAEOE) nucleotides, 2′-O-aminopropyl(2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a internal-hairpin natural or modified nucleobase; or a combination thereof. For example, the stem portion and/or the loop portion of the internal-hairpin comprises a 2′-O-methyl modified nucleotide. In another example, the stem portion and/or the loop portion of the internal-hairpin comprises a 2′-fluoro modified nucleotide.

The internal-hairpin can also comprise one or more (e.g., 1, 2, 3, 4, 5 or more) internal-hairpin natural or modified nucleobases. The internal-hairpin natural or modified nucleobase can be present in the stem portion or the loop portion of the internal-hairpin. In some embodiments, the stem portion of the internal-hairpin comprises one or more (e.g., 1, 2, 3, 4, 5 or more) internal-hairpin natural or modified nucleobases. In another embodiment, the loop portion of the internal-hairpin comprises one or more (e.g., 1, 2, 3, 4, 5 or more) internal-hairpin natural or modified nucleobases. In yet some other embodiments, both the stem portion and the loop portion of the internal-hairpin comprises one or more (e.g., 1, 2, 3, 4, 5 or more) independently selected internal-hairpin natural or modified nucleobases.

The internal-hairpin can also comprise at least one (e.g., 1, 2, 3, 4, 5, or more) modified internucleoside linkages. Exemplary modified internucleoside linkages are described herein and include, but are not limited to, phosphorothioate and MMI linkages. Like other modifications, a modified internucleotide linkage can be present in the stem portion and/or the loop portion of the internal-hairpin.

In some embodiments, the stem portion of the internal-hairpin comprises one or more (e.g., 1, 2, 3, 4, 5 or more) modified internucleoside linkages (e.g., phosphorothioate and/or MMI linkages). In another embodiment, the loop portion of the internal-hairpin comprises one or more (e.g., 1, 2, 3, 4, 5 or more) modified internucleoside linkages (e.g., phosphorothioate and/or MMI linkages). In yet some other embodiments, both the stem portion and the loop portion of the internal-hairpin comprises one or more (e.g., 1, 2, 3, 4, 5 or more) independently selected modified internucleoside linkages (e.g., phosphorothioate and/or MMI linkages). In some embodiments, the internal-hairpin comprises a modified internucleoside linkage (e.g., phosphorothioate and/or MMI linkage) between the stem portion and the loop portion of the internal-hairpin.

Without wishing to be bound by a theory, increasing the stability, e.g., stability of the stem of the internal-hairpin can provide increased or higher nucleic acid modification, e.g., editing activity and/or efficiency. Accordingly, in some embodiments, the stem portion of the internal-hairpin comprises a duplex stabilizing modification. Some exemplary duplex stabilizing modifications include, but are not limited to, 2′-F nucleotides, 2′-OMe nucleotides, 2′-methoxyethyl nucleotides, 2,6-diaminopurine nucleotides, 5-methyl cytidine, N4-ethyl cytidine, 5-propynyl cytidine, 5-propynyl uridine, 5-hydroxybutynl-2′-deoxyuridine, 8-aza-7-deazaguanosine, a locked nucleic acid (LNA), and/or covalent cross-linking of two strands of the duplex. In some embodiments, the stem portion comprises a covalent linkage between two nucleotides, optionally the covalent linkage is at a position other than 5′ and 3′ of the nucleotides.

In some embodiments, a duplex stabilizing modification comprises changing an A:U base pair in the stem of the internal-hairpin to a G: C base-pair. For example, one or more, e.g., 1, 2, 3, 4, 5 or more A:U base pairs in the stem of the internal-hairpin ton G: C base-pairs. It is known that guanine tetrads are formed by sequences rich in guanine, such as GGGGC. Thus, in order to reduce formation of guanine tetrads, G and C in the G: C base-pair replacing a A:U base-pair are positioned so that there are no more than 5, preferably no more than 4, and more preferably no more than 3 consecutive the sequence of the stem.

In some embodiments, a duplex stabilizing modification comprises a A:U base pair in the stem of the internal-hairpin to a G: C base-pair. For example, one or more, e.g., 1, 2, 3, 4, 5, 6, 7 or more A:U base pairs in the stem of the internal-hairpin ton G: C base-pairs. In some embodiments, a duplex stabilizing modification comprises replacing a A:U base pair in the stem of the 5′-hairpin with a G: C base-pair. For example, one or more, e.g., 1, 2, 3, 4, 5, 6, 7 or more A:U base pairs in the stem of the 5′-hairpin ton G: C base-pairs. In some embodiments, a duplex stabilizing modification comprises replacing mismatch in the stem of the 5′-hairpin with a G: C or A:U base-pair. It is known that guanine tetrads are formed by sequences rich in guanine, such as GGGGC. Thus, in order to reduce formation of guanine tetrads, G and C in the G: C base-pair replacing a A:U base-pair or a mismatch are positioned so that there are no more than 5, preferably no more than 4, and more preferably no more than 3 consecutive G in the sequence of the stem.

In some embodiments, a G: U base-pair in the stem of the internal-hairpin of a wild-type or parent ωRNA conserved region is replaced with a G: C base-pair. For example, the U in the mismatch is replaced by C, or the U is replaced by G and the G is replaced by C.

In some embodiments of any one of the aspects described herein, one or more nucleotides in the internal-hairpin can base pair with nucleotides in another part of the sequence and form a pseudoknot structure. For example, one or more nucleotides in the pin-loop of the internal loop can base pair with nucleotides in another portion of the conserved region to from a pseudoknot structure. The nucleotides involved in forming the pseudoknot structure can be modified to stabilize the pseudoknot structure. For example, one or more A:U base-pairs or mismatches in the pseudoknot structure can be replaced with G: C base-pairs. In some embodiments, an A:U base-pair in the pseudoknot structure can be replaced with a U: U base-pair.

In some embodiments, one or more (e.g., 2, 3, 4, 5, 6, 7, 8, 9, 10, 11 or more) nucleotides are inserted between immediately 5′- to the first nucleotide in the pin-loop of the internal hairpin that is involved in forming the pseudoknot structure.

### Deletions at 3′-End

In some embodiments, one or more nucleotides present at the 3′-end of the wild-type or parent ωRNA conserved region, that are not involved in forming the 3′-hairpin are deleted. For example, 10 to about 30 nucleotides present at the 3′-end of the wild-type or parent ωRNA conserved region, that are not involved in forming the 3′-hairpin are deleted. In some embodiments, 1, 2, 3, 4, 5, 6, 7, 8, 9, 10, 11, 12, 13, 14, 15, 16, 17, 18, 1, 20, 21, 22, 23, 24, 25, 26, 27, 28, 29 or 30 nucleotides present at the 3′-end of the wild-type or parent ωRNA conserved region, that are not involved in forming the 3′-hairpin are deleted.

### Length of Conserved Region

In some embodiments, the ωRNA comprises a conserved region that is at least 45 or more nucleotides shorter than the conserved region of the parent or wild-type ωRNA. In some embodiments, the ωRNA comprises a conserved region that is from about 50 to about 90 nucleotides shorter than the conserved region of the parent or wild-type ωRNA. For example, the ωRNA comprises a conserved region that is from about 55 to about 85 nucleotides shorter than the conserved region of the parent or wild-type ωRNA. In some embodiments, the ωRNA comprises a conserved region that is about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, or about 90, nucleotides shorter than the wild-type or parent ωRNA. For example, the ωRNA comprises a conserved region that is 60, 61, 62, 63, 64, 65, 66, 67, 68, 69, 70, 71, 72, 73, 74, 75, 76, 77, 78, 79, or 80 nucleotides shorter than the wild-type or parent ωRNA.

In some embodiments, the ωRNA comprises a conserved region less than 200 nucleotides in length. For example, the ωRNA comprises a conserved region comprising 195 or less, 190 or less, 185 or less, 180 or less, 175 or less, 170 or less, 165 or less, 160 or less, 155 or less, 150 or less, 145 or less, 140 or less, or 135 or less nucleotides in length. In some embodiments, the conserved region is from about 125 nucleotides to about 195 nucleotides in length. For example, the ωRNA comprises a conserved region comprising from about 130 nucleotides to about 190 nucleotides in length, such as from about 135 nucleotides to about 180 nucleotides or from about 140 nucleotides to about 170 nucleotides. In some embodiments of any one of the aspects described herein, the conserved region is about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, or about 160 nucleotides in length. For example, the conserved region of ωRNA is 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 nucleotides in length. In some embodiments, the conserved region of ωRNA is 120, 121, 122, 123, 124, 125, 126, 127, 128, 129, 130, 131, 132, 133, 134, or 135 nucleotides in length.

In some embodiments of any one of the aspects described herein, the conserved region of a ωRNA described herein comprises a nucleotide sequence having at least 70%, at least 75%, at least 80%, at least 85%, at least 88%, at least 90%, at least 92%, at least 95%, at least 98%, at least 99% or 100% sequence identity to the sequence:

(SEQ ID NO: 10)
GUCAAUAACCCGAAAGGGUUACUGAGCAGAGCAGUGACAUGUCAU
UCGCCGCGGGGUGAUUCCAAGCUCCGCGCUCCGGCUAGACAUGCC
CAUGCUAUGGAAACUUUAACGGUGAAAACCGGCUUACAACAAA

A conserved region consisting of SEQ ID NO: 10 is also referred to a M71 conserved region. In some embodiments, the conserved region of a ωRNA comprises the sequence of M71 conserved region, wherein the conserved region further comprises at least one, at least two, at least three, at least four, at least five, at least at least six, seven, at least eight, at least nine or at modifications from Table 19 in the Examples, where each T is replaced by U.

### Guide Sequence (Spacer, Variable Region)

In some embodiments, guide nucleic acid, e.g., ωRNA comprises a guide sequence, also referred to as a spacer or spacer sequence herein. In some embodiments, the guide sequence comprises a nucleotide sequence that is complementary to a portion of a target nucleic acid. The guide sequence can be referred to as a “cr sequence” herein. Generally, the spacer or guide sequence comprises a sequence of at least 10 nucleotides that is complementary to a target sequence. In some embodiments, the spacer or guide sequence comprises a sequence of from 10 nucleotides to 50 nucleotide that is complementary to a target sequence. For example, the spacer or guide sequence comprises a sequence of 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

The spacer and the conserved region of the guide nucleic acid, e.g., ωRNA can be located in any preferred position to each other. In some embodiments, the spacer or guide sequence is at 5′-end of the conserved region of the guide nucleic acid, e.g., RNA. In other words, 3′-end of the spacer is lined to the 5′-end of the conserved region of the guide nucleic acid, e.g., ωRNA. The spacer and the conserved region can be linked directly (e.g., by a bond or modified or unmodified internucleoside linkage) or by a nucleotide sequence comprising from 1 to 25 nucleotides or by a non-nucleotide linker. In some preferred embodiments, the 3′-end of the spacer is linked directly to the 5′-end of the conserved region by a modified (e.g., phosphorothioate, imidp or MMI linkage) or unmodified (e.g., phosphodiester) internucleoside linkage)

The nucleotide sequence of the spacer (i.e., guide sequence) can be chosen to direct site-specific binding to a target sequence of a target nucleic acid. The spacer can comprise an engineered heterologous sequence. Generally, the spacer is from about 10 to about 50 nucleotides in length. In some embodiments, the spacer is at least 15 nucleotides in length. For example, the spacer is at least 16 nucleotides, at least 17 nucleotides, at least 18 nucleotides, at least 19 nucleotides, at least 20 nucleotides, at least 21 nucleotides, or at least 22 nucleotides in length. In some embodiments, the spacer is from about 15 to about 17 nucleotides, from about 18 to about 20 nucleotides, from about 21 to about 23 nucleotides, from about 24 to about 26 nucleotides, from about 27 to about 29 nucleotides, from about 30 to about 32 nucleotides, from about 33 to about 35 nucleotides, from about 36 to about 38 nucleotides, from about 39 to about 41 nucleotides, from about 42 to about 44 nucleotides, from about 45 to about 47 nucleotides, or from about 48 to about 50 nucleotides in length. In some embodiments, the spacer is from 15 to 17 nucleotides, from 15 to 23 nucleotides, from 15 to 30 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides in length. In some embodiments, the spacer is 15, 16, 17,18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, or 50 nucleotides in length. In some preferred embodiments, the spacer is from 16 to 23 nucleotides (e.g., 16, 17, 18, 19, 21, 22 or 23 nucleotides) in length. For example, the spacer is 19, 20 or 21 nucleotides in length.

In some embodiments, the spacer or guide sequence comprises from about 10 to about 100 nucleotides and a sequence of at least 10 nucleotides that is complementary to a target sequence. For example, the spacer or guide sequence comprises from about 10 to about 100 nucleotides and a sequence of 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

In some embodiments, the spacer or guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long and comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

To reduce off-target interactions, e.g., to reduce the spacer interacting with a non-target sequence, mutations can be introduced so that the engineered, non-natural DNA and RNA targeting complexes described herein can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a spacer sequence and its corresponding target sequence is greater than 90%, 90.5%, 91%, 91.5%, 92%, 92.5%, 93%, 93.5%, 94.5%, 95%, 95.5%, 96%, 96.5%, 97%, 97.5%, 98%, 98.5%, 99%, 99.5%, or 99.9%. In some embodiments, the degree of complementarity between the spacer sequence and the target nucleic acid is 100%. Degree of complementarity between different nucleic acid sequence can be determined with the use of any suitable algorithm for aligning sequences, non-limiting example of which include the Smith-Waterman algorithm, the Needleman-Wunsch algorithm, algorithms based on the Burrows-Wheel er Transform (e.g., the Burrows Wheeler Aligner), ClustalW, Clustal X, BLAT, Novoalign (Novocraft Technologies; available at novocraft.com), ELAND (Illumina, San Diego, CA), SOAP (available at soap.genomics.org.cn), and Maq (available at maq.sourceforge.net).

For all embodiments of the systems and methods described herein, it is known in the field that complete complementarity is not required for hybridization or binding as described herein, provided that there is sufficient complementarity to be functional. Accordingly, in some embodiments, the nucleotide sequence of the spacer comprises at least one (e.g., 2, 3, 4 or 5) mismatch with the target nucleic acid. In some embodiments, the spacer sequence is designed to have a stretch of two or more adjacent mismatching nucleotides with the target nucleic acid.

Without wishing to be bound by a theory, modulations of cleavage efficiency can be exploited by introducing mismatches, e.g., one or more mismatches, such as 1 or 2 mismatches between a spacer sequence and a target sequence, including the position of the mismatch along the spacer/target. The more central (i.e., not at the 3′ or 5′ ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer sequence and target sequence can be introduced in the spacer sequences.

In some embodiments, the guide nucleic acid comprises a direct repeat (DR) sequence linked to the guide sequence. The direct repeat sequence comprises at least one hairpin or stem loop structure. Generally, the direct repeat sequence has a minimum length of 16 nucleotides and one hairpin or stem loop. In some embodiments, the direct repeat sequence has a length longer than 16 nucleotides, preferably more than 17 nucleotides, and has two or more hairpin or stem loops. In some embodiments, the hairpin or the stem loop structure comprises at least 5, preferably 7-20 nucleotides. The direct repeat sequence can be 3′ of the spacer (guide sequence). Alternatively, the direct repeat sequence can be 5′ of the spacer (guide sequence). In some embodiments, the spacer is flanked by a direct repeat sequence at its 5′ and 3′ ends. It is noted that a guide nucleic acid comprising a spacer flanked by a direct repeat sequence at its 5′ and 3′ ends (DR-spacer-DR) structure is typical of precursor crRNA (pre-crRNA). In some embodiments, the guide nucleic acid comprises a truncated direct repeat sequence and a spacer sequence, which is typical of processed or mature crRNA.

The direct repeat can be at least 10 nucleotides in length. For example, the direct repeat can be at least 11 nucleotides, or at least 12 nucleotides, or at least 13 nucleotides, or at least 14 nucleotides, or at least 15 nucleotides, or at least 16 nucleotides, or at least 17 nucleotides, or at least 18 nucleotides, or at least 19 nucleotides, or at least 30 nucleotides in length. In some embodiments, the direct repeat comprises the sequence NACACC, proximal to the spacer, where N is G or T.

The guide nucleic acid can further include a tracr sequence for complexing with the protein effector polypeptide. The term “tracr sequence”, as used herein, can generally refer to a nucleic acid with at least about 5%, 10%, 20%, 30%, 40%, 50%, 60%, 70%, 80%, 90%, 95%, or 100% sequence identity and/or sequence similarity to a wild type exemplary tracrRNA sequence. In some embodiments of any one of the aspects described herein, the tracr sequence comprises one or more, e.g., 1, 2, 3, 4 or more hairpins or stem loops. In some embodiments, the tracr sequence comprises a sequence predicted to comprise at least two hairpins comprising less than 5 base-paired ribonucleotides. The spacer and the tracr sequences can be linked to each other via a hairpin or stem loop structure. The tracr sequence can be 5 or more nucleotides in length. For example, the tracr sequence can be 6, 7, 8, 9, 10, 11, 12, 13, 14, 15, 16, 17, 18, 19, 20, 25, 30, 40, 50, or more nucleotides in length.

The guide nucleic acid can be single-stranded or double-stranded. In some embodiments, the guide nucleic acid is single polynucleotide chain, and optionally comprises double-stranded regions. Guide nucleic acid that comprise a single polynucleotide chain can be referred to as a “single guide nucleic acid.” It is noted that when the guide nucleic acid is a single polynucleotide chain, the spacer and the tracr sequence can be located in any preferred position to each other. For example, the spacer can be located 5′ of the tracr sequence. Alternatively, the spacer can be located 3′ of the tracr sequence. In some preferred embodiments, the spacer is 5′ of the tracr sequence.

In some embodiments, the spacer and the tracr sequence of the guide nucleic acid are present in separate polynucleotide chains. Optionally, the separate polynucleotides chains are partially hybridized to each other. Guide nucleic acid that comprise two polynucleotide chains can be referred to as a “double guide nucleic acid.” Generally, the 3′-end of the polynucleotide chain comprising the spacer hybridized with the 5′-end of the polynucleotide chain comprising the tracr sequence. The polynucleotide chain comprising the spacer is also referred to as “crRNA” herein, and the polynucleotide chain comprising the tracr sequence is also referred to as “tracrRNA” herein.

In some embodiments, the spacer or guide sequence is linked to a direct repeat and the tracr sequence is linked to an anti-repeat sequence. The direct repeat and anti-repeat sequences are substantially complementary to each other and can hybridize with each other to form a double-stranded region. The double-stranded region can be at least 8, or at least 9, or at least 10, or at least 11, or at least 12 base-pairs in length.

The guide nucleic acid can also include one or more protein binding domains, e.g., for binding with or recruiting one or more gene effectors, gene activators, or gene repressors. Generally, the protein binding domain of the guide nucleic acid comprises a scaffold that is capable of binding with a protein. In some embodiments, the protein binding domain can be an aptamer. Aptamers are oligonucleotide or peptide molecules that can bind to a specific target molecule. The aptamers can be specific to gene effectors, gene activators, or gene repressors. In some embodiments, the aptamers can be specific to a protein, which in turn is specific to and recruits/binds to specific gene effectors, gene activators, or gene repressors. The effectors, activators, or repressors can be present in the form of fusion proteins. In some embodiments, the guide nucleic acid comprises two or more aptamer sequences that are specific to the same adaptor proteins. In some embodiments, the two or more aptamer sequences are specific to different adaptor proteins. The adaptor proteins can include, but are not limited to, MS2, PP7, QB, F2, GA, fr, JP501, M12, R17, BZ13, JP34, JP500, KUI, M11, MX1, TW18, VK, SP, FI, ID2, NL95, TW19, AP205, ΦCb5, ΦCb8r, ΦCb12r, ΦCb23r, 7s, and PRR1. Accordingly, in some embodiments, the aptamer is selected from binding proteins specifically binding any one of the adaptor proteins as described herein. In some embodiments, the aptamer sequence is a MS2 binding loop, QB binding loop, or PP7 binding loop. A detailed description of aptamers can be found, e.g., in Nowak et al., “Guide RNA engineering for versatile Cas9 functionality,” Nucl. Acid. Res., 2016 Nov. 16; 44 (20): 9555-9564; and WO2016205764, which are incorporated herein by reference in their entirety.

In an embodiment, the guide nucleic acid further comprises an extension to add an RNA template. In some embodiments, a “protector nucleic acid” can be hybridized to a sequence of the guide nucleic acid, wherein the “protector nucleic acid” is nucleic acid strand complementary to the 3′ end of the guide nucleic acid to thereby generate a partially double-stranded guide nucleic acid. In some embodiments, protecting mismatched bases (i.e., the bases of the guide nucleic acid which do not form part of the guide sequence) with a perfectly complementary protector sequence decreases the likelihood of a target sequence binding to the mismatched basepairs at the 3′ end of the guide nucleic acid. In some embodiments, additional sequences comprising an extended length can also be present within the guide nucleic acid such that the guide nucleic acid comprises a protector sequence within the guide nucleic acid molecule. This “protector sequence” ensures that the guide nucleic acid molecule comprises a “protected sequence” in addition to an “exposed sequence” (comprising the part of the guide nucleic acid sequence hybridizing to the target sequence). In some embodiments, the guide nucleic acid is modified by the presence of the protector nucleic acid to comprise a secondary structure such as a hairpin. Advantageously there are three or four to thirty or more, e.g., about 10 or more, contiguous base pairs having complementarity to the protected sequence, the guide nucleic acid sequence or both. It is advantageous that the protected portion does not impede thermodynamics of the protein effector polypeptide and related system interacting with its target. By providing such an extension including a partially double stranded guide nucleic acid, the guide nucleic acid is considered protected and results in improved specific binding of the protein effector polypeptide/guide nucleic acid, while maintaining specific activity.

In some embodiments, the guide nucleic acid structure comprises a sequence predicted to comprise a hairpin consisting of a stem and a loop, wherein the stem comprises at least 10, at least 12 or at least 14 base-paired ribonucleotides, and an asymmetric bulge within 4 base pairs of the loop.

In some embodiments, the guide nucleic acid comprises a sequence predicted to comprise a hairpin with an uninterrupted base-paired region comprising at least 8 nucleotides of a guide sequence and at least 8 nucleotides of a tracr sequence, and wherein the tracr sequence comprises, from 5′ to 3′, a first hairpin and a second hairpin, wherein the first hairpin has a longer stem than the second hairpin.

The guide nucleic acids can be chemically synthesized or can be generated as components of inducible systems. The inducible nature of the systems allows for spatiotemporal control of gene editing or gene expression. In some embodiments, the stimuli for the inducible systems include, e.g., electromagnetic radiation, sound energy, chemical energy, and/or thermal energy. For example, the transcription of guide nucleic acids can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in their entirety.

The architecture of multiple RNA guides is known in the art. See, for example, International Publication Nos. WO 2014/093622 and WO 2015/070083, the entire contents of each of which are incorporated herein by reference). Thus, in some embodiments, the engineered, non-natural DNA and RNA targeting complexes described herein include multiple guide nucleic acids (e.g., two, three, four, five, six, seven, eight, or more guide nucleic acids). Sequences for RNA guides from multiple CRISPR systems are known in the art and can be searched using public databases (see, e.g., Grissa et al. (2007) Nucleic Acids Res. 35 (web server issue): W52-7; Grissa et al. (2007) BMC Bioinformatics 8:172; Grissa et al. (2008) Nucleic Acids Res. 36 (web server issue): W145-8; and Moller and Liang (2017) PeerJ 5: e3788; see also the CRISPR database available at: crispr.i2bc.paris-saclay.fr/crispr/BLAST/CRISPRsBlast.php; and MetaCRAST available at: github.com/molleraj/MetaCRAST).

In some embodiments, an engineered, non-natural DNA and RNA targeting complexes described herein comprises two different guide nucleic acids targeting a first region and a second region in a target nucleic acid. In some embodiments, the first region is 5′ to the second region. In some other embodiments, the first region is 3′ to the second region.

In some embodiments, two different guide nucleic acids can be encoded by a single polynucleotide. For example, the polynucleotide sequence encoding a guide nucleic acid comprises a CRarray comprising two or more guide sequences.

The guide nucleic acid sequences can be modified in a manner that allows for formation of a complex comprising the protein effector polypeptide and the guide nucleic acid, and successful binding to the target, while at the same time not allowing for successful nuclease activity. These modified guide sequences are referred to as “dead guides” or “dead guide sequences.” These dead guides or dead guide sequences can be catalytically inactive or conformationally inactive with regards to nuclease activity. Dead guide sequences are typically shorter than respective guide sequences that result in active target (e.g., RNA or DNA) cleavage. In some embodiments, dead guides are 5%, 10%, 20%, 30%, 40%, or 50%, shorter than respective guide nucleic acids that have nuclease activity. Dead guide sequences of guide nucleic acids can be from 13 to 15 nucleotides in length (e.g., 13, 14, or 15 nucleotides in length), from 15 to 19 nucleotides in length, or from 17 to 18 nucleotides in length (e.g., 17 nucleotides in length). Thus, in one aspect, the disclosure provides engineered, non-natural DAN and RNA targeting system or composition comprising a functional protein effector polypeptide as described herein, and a guide nucleic acid, wherein the guide nucleic acid includes a dead guide sequence whereby the guide nucleic acid is capable of hybridizing to a target sequence such that the complex is directed to a genomic locus of interest, e.g., in a cell without detectable cleavage activity. A detailed description of dead guides is described, e.g., in WO 2016094872, which is incorporated herein by reference in its entirety.

In some embodiments of any one of the aspects described herein the guide RNA is an ωRNA described herein.

Also provided herein are DNA and RNA targeting systems comprising an IscB protein (or a fusion protein) described herein and a guide RNA nucleic acids (e.g., a guide RNA or ωRNA). Also provided herein are DNA and RNA targeting systems comprising: guide RNA nucleic acids (e.g., a guide RNA or ωRNA) described herein and a protein effector polypeptide.

In some embodiments, a DNA and RNA targeting complex described herein exhibits a lower or decreased off-target activity, i.e., modification of a non-target sequence, as compared to the off-target activity of a complex comprising a wild-type IscB and wild-type gRNA. For example, the off-target activity of a DNA and RNA targeting complex described herein, is at least at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold lower than the off-target activity of a complex comprising a wild-type IscB and wild-type gRNA.

### Conservative Variants

In the various embodiments described herein, it is further contemplated that variants (naturally occurring or otherwise), alleles, homologs, conservatively modified variants, and/or conservative substitution variants of any of the polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

The term “amino acid substitution” refers to the replacement of at least one existing amino acid residue in a predetermined or native amino acid sequence with a different “replacement” amino acid. A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Conservative substitution tables providing functionally similar amino acids are also available from a variety of references (see, for e.g., Creighton, Proteins: Structures and Molecular Properties (W H Freeman & Co.; 2nd edition (December 1993)). The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine(S), Threonine (T); and (8) 8) Cysteine (C), Methionine (M). Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

### Split Protein Effector Polypeptides

The present disclosure also provides a split version of the protein effector polypeptides described herein. The split version of the protein effector polypeptides can be advantageous for delivery. In some embodiments, the protein effector polypeptides are split into two parts, which together substantially comprises a functional activity of the full length of the protein effector polypeptide. The split can be done in a way that the catalytic domain(s) are unaffected. The protein effector polypeptides can function as a nuclease (e.g., endonuclease) or can be inactivated enzymes, which are essentially DNA or RNA-binding proteins with very little or no catalytic activity (e.g., due to mutation(s) in its catalytic domains).

In some embodiments, the nuclease lobe and a-helical lobe are expressed as separate polypeptides. Although the lobes do not interact on their own, the guide nucleic acid recruits them into a surveillance complex that recapitulates the activity of full-length protein effector polypeptides and catalyzes site-specific DNA or RNA cleavage. The use of a modified guide nucleic acid can abrogate split-polypeptide activity by preventing dimerization, allowing for the development of an inducible dimerization system. The split CRISPR enzymes are described, e.g., in Wright, Addison V., et al. “Rational design of a split-Cas9 enzyme complex,” Proc. Natl. Acad. Sci., 112.10 (2015): 2984-2989, which is incorporated herein by reference in its entirety.

In some embodiments, the split protein effector polypeptide can be fused to a dimerization partner, e.g., by employing rapamycin sensitive dimerization domains. This allows the generation of a chemically inducible protein effector polypeptides for temporal control of activity. The protein effector polypeptides can thus be rendered chemically inducible by being split into two fragments and rapamycin-sensitive dimerization domains can be used for controlled reassembly of the protein effector polypeptide.

The split protein effector polypeptides can be induced to combine to Forming a functional domain, e.g., nuclease domain from split protein effector polypeptides can be inducible, e.g., light inducible or chemical inducible. Without wishing to be bound by a theory, this mechanism allows for activation of the functional domain in the protein effector polypeptides with a known trigger. Light inducibility can be achieved by various methods known in the art, e.g., by designing a fusion complex wherein CRY2 PHR/CIBN pairing is used in split protein effector polypeptides. See, for example, Konermann et al. “Optical control of mammalian endogenous transcription and epigenetic states,” Nature, 500.7463 (2013): 472). Chemical inducibility can be achieved, e.g., by designing a fusion complex wherein FKBP/FRB (FK506 binding protein/FKBP rapamycin binding domain) pairing is used in split protein effector polypeptides. Rapamycin is required for forming the fusion complex, thereby activating the protein effector polypeptides (see, e.g., Zetsche, Volz, and Zhang, “A split-Cas9 architecture for inducible genome editing and transcription modulation,” Nature Biotech., 33.2 (2015): 139-142).

The split point is typically designed in silico and cloned into the constructs. During this process, mutations can be introduced to the split enzyme and non-functional domains can be removed. In some embodiments, the two parts or fragments of the split protein effector polypeptide (i.e., the N-terminal and C-terminal fragments), can form a full protein effector polypeptide, comprising, e.g., at least 70%, at least 80%, at least 90%, at least 95%, or at least 99% of the sequence of the wild-type or full-length protein effector polypeptide.

### Self-Inactivating Protein Effector Polypeptides

The protein effector polypeptides described herein can be designed to be self-activating or self-inactivating. In some embodiments, the protein effector polypeptides are self-inactivating. For example, a target sequence can be introduced into the protein effector polypeptide coding constructs. Thus, the protein effector polypeptides can cleave the target sequence, as well as the construct encoding the enzyme thereby self-inactivating their expression. See, for example, Epstein, Benjamin E., and David V. Schaffer. “Engineering a Self-Inactivating CRISPR System for AAV Vectors,” Mol. Then, 24 (2016): S50, which is incorporated herein by reference in its entirety.

In some other embodiments, an additional guide nucleic acid, e.g., expressed under the control of a weak promoter (e.g., 7SK promoter), can target the nucleic acid sequence encoding the protein effector polypeptide to prevent and/or block its expression (e.g., by preventing the transcription and/or translation of the nucleic acid). The transfection of cells with vectors expressing the protein effector polypeptide and guide nucleic acid(s) that target the nucleic acid encoding the protein effector polypeptide can lead to efficient disruption of the nucleic acid encoding the protein effector polypeptide and decrease the levels of protein effector polypeptide, thereby limiting the nucleic acid modifying activity.

In some embodiments, the genome editing activity of the protein effector polypeptides can be modulated through endogenous RNA signatures (e.g., miRNA) in mammalian cells. The protein effector polypeptide switch can be made by using a miRNA-complementary sequence in the 5′-UTR of mRNA encoding the protein effector polypeptide. The switches selectively and efficiently respond to miRNA in the target cells. Thus, the switches can differentially control the genome editing by sensing endogenous miRNA activities within a heterogeneous cell population. Therefore, the switch systems can provide a framework for cell-type selective genome editing and cell engineering based on intracellular miRNA information (Hirosawa, Moe et al. “Cell-type-specific genome editing with a microRNA-responsive CRISPR-Cas9 switch,” Nucl. Acids Res., 2017 Jul. 27; 45 (13): e118).

### Nucleic Acids and Vectors Encoding Polypeptides and Guides

The disclosure also provides a polynucleotide encoding a protein effector polypeptide and/or a guide nucleic acid described herein. The skilled person will understand that, due to the degeneracy of the genetic code, a given polypeptide can be encoded by different polynucleotides. These “variants” are encompassed herein.

In some embodiments, a polynucleotide encoding a protein effector polypeptide and/or a guide nucleic acid described herein is comprised in a vector. In some embodiments, a nucleic acid sequence encoding a protein effector polypeptide and/or a guide nucleic acid described herein, or any part thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. The term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

A vector can include, but is not limited to, a cloning vector, an expression vector, a plasmid, phage, transposon, cosmid, chromosome, virus, virion, etc. Some exemplary vectors include pGEX, pMAL, pRIT5, E. coli expression vectors (e.g., pTrc, pET1 id, yeast expression vectors (e.g., pYepSec1, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC).

A vector can comprise one or more (e.g., 1, or at least 2, at least 3, at least 4, at least 5, at least 6, at least 7, at least 8, at least 9, at least 10 or more) protein effector polypeptide encoding sequence(s), and/or one or more (e.g., 1, or at least 2, at least 3, at least 4, at least 5, at least 6, at least 7, at least 8, at least 9, at least 10, at least 12, at least 14, at least 16, at least 32, at least 48, at least 50 or more) guide nucleic acid encoding sequences. In a single vector there can be a promoter for each RNA coding sequence. Alternatively, or additionally, in a single vector, there can be a promoter controlling (e.g., driving transcription and/or expression) multiple RNA encoding sequences.

In some embodiments, the vector comprises: a first regulatory element operably linked to a nucleotide sequence encoding a protein effector polypeptide described herein, and a second regulatory element operably linked to a nucleotide sequence encoding a guide nucleic acid described herein.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. [0561] Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the P-actin promoter, the phosphoglycerol kinase (PGK) promoter, and the EF1a promoter.

In some embodiments, the promoter is an inducible promoter. In some embodiments, the promoter is a cell-specific promoter, such as Syn and CamKIIa for neuronal cell types, or thyroxine binding globulin (TBG) for hepatocyte expression. In some embodiments, the promoter is an organism-specific promoter. Suitable promoters are known in the art and include, for example, a pol I promoter, a pol II promoter, a pol III promoter. In some embodiments, short RNAs such as the guide nucleic acid are effectively expressed using a pol III promoter, which includes a U6 promoter, a H1 promoter, a 7SK promoter. In some embodiments, the promoter is prokaryotic, such as a T7 promoter. In some embodiments, the promoters are eukaryotic and include retroviral Rous sarcoma virus LTR promoter, a cytomegalovirus (CMV) promoter, a SV40 promoter, a dihydrofolate reductase promoter, a b-actin promoter, elongation factor 1 alpha promoter, elongation factor 1 alpha short promoter, SV40 promoter, and the synthetic CAG promoter. In some embodiments, the termination signals for induction of mRNA polyadenylation include, but are not limited to, SV40, hGH, and bGH.

In some embodiments, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments, the sequence encoding the protein effector polypeptide and/or a guide nucleic acid described herein is codon-optimized, e.g., the native or wild-type sequence of the nucleic acid sequence has been altered or engineered to include alternative codons such that altered or engineered nucleic acid encodes the same polypeptide or expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell. In some embodiments, the vector and/or nucleic acid sequence described herein is codon-optimized for expression in a yeast or yeast cell. In some embodiments, the vector and/or nucleic acid sequence described herein is codon-optimized for expression in a bacterial cell. In some embodiments, the vector and/or nucleic acid sequence described herein is codon-optimized for expression in an E. coli cell.

As used herein, the term “expression vector” refers to a vector that directs expression of an RNA or polypeptide from sequences operably linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

In some embodiments, the vector can be a viral vector. As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art. Some exemplary viral vectors amenable to the invention include, but are not limited to, adeno associated virus (AAV such as AAV-1, AAV-2, AAV-3, AAV-4, AAV-5, AAV-6, AAV-8, and AAV-9), adenoviruses lentiviruses (such as human immunodeficiency virus and equine infectious anemia virus), plat viral vectors (such as geminivirus (e.g., cabbage leaf curl virus, bean yellow dwarf virus, wheat dwarf virus, tomato leaf curl virus, maize streak virus, tobacco leaf curl virus, and tomato golden mosaic virus), nanovirus (e.g., Faba bean necrotic yellow virus), tobravirus (e.g., tobacco rattle virus, tobacco mosaic virus), potexvirus (e.g., potato virus X), and hordeivirus (e.g., barley stripe mosaic virus).

In some embodiments, the vector comprises: a first regulatory element operably linked to a nucleotide sequence encoding a protein effector polypeptide described herein, and a second regulatory element operably linked to a nucleotide sequence encoding a guide nucleic acid described herein.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. [0561] Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the P-actin promoter, the phosphoglycerol kinase (PGK) promoter, and the EF1a promoter.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

It is noted that any reference to a protein effector polypeptide or a guide nucleic acid includes polynucleotides or vectors encoding same. Particularly, any reference to a protein effector polypeptide or a guide nucleic acid includes polynucleotides or vectors encoding same, where the polynucleotide or vector is operably linked to one or more regulatory elements, such as a promoter.

### Modified Cells

The disclosure also provides cell comprising one or more components of the engineered, non-natural DNA and RNA targeting complexes described herein or a polynucleotide (e.g., a vector) encoding the same. Also provided herein are cells modified by the engineered, non-natural DNA and RNA targeting complexes described herein, and cell cultures, tissues, organs, organism comprising such cells or progeny thereof.

A modified cell or a cell comprising one or more components of the engineered, non-natural DNA and RNA targeting complexes described herein or a polynucleotide (e.g., a vector) encoding the same can be a prokaryotic cell or a eukaryotic cell. The cell can be a mammalian cell. The mammalian cell can be a non-human primate, bovine, porcine, rodent or mouse cell. The cell can be a non-mammalian eukaryotic cell such as poultry, fish or shrimp. The cell can be a therapeutic T cell or antibody-producing B-cell. The cell can also be a plant cell. The plant cell can be of a crop plant such as cassava, corn, sorghum, wheat, or rice. The plant cell can also be of an algae, tree or vegetable. The modification introduced to the cell using the DNA and RNA targeting systems, compositions and methods described herein can be such that the cell and progeny of the cell are altered for improved production of biologic products such as an antibody, starch, alcohol or other desired cellular output. The modification introduced to the cell using the DNA and RNA targeting systems, compositions and methods described herein can be such that the cell and progeny of the cell include an alteration that changes the biologic product produced.

### Synthesis of Protein Effector Polypeptides and Guide Nucleic Acids

Recombinant methods can be used for producing protein effector polypeptides and/or guide nucleic acids described herein. For example, host cells can be employed in a method of producing a protein effector polypeptide and/or a guide nucleic acid described herein. Accordingly, the disclosure also provides a host cell comprising a polynucleotide or a plasmid or vector encoding a protein effector polypeptide and/or a guide nucleic acid described herein. A host cell can be a prokaryotic or eukaryotic host cell. Exemplary host cells include, but are not limited to, bacterial cells, yeast cells, plant cell, animal (including insect) or human cells.

Generally, the method comprises: culturing a host cell comprising a polynucleotide described herein or a plasmid or vector described herein under conditions such that the protein effector polypeptide and/or the guide nucleic acid is expressed; and optionally recovering the protein effector polypeptide and/or the guide nucleic acid from the culture medium. The protein effector polypeptide and/or the guide nucleic acid can be concentrated and purified by a variety of biochemical and chromatographic methods, including methods utilizing differences in size, charge, hydrophobicity, solubility, specific affinity, etc. between the desired product (e.g., the protein effector polypeptide and/or the guide nucleic acid) and other substances in the cell culture medium. In some embodiments, the protein effector polypeptide and/or the guide nucleic acid is secreted from the host cells.

The protein effector polypeptide described herein can be produced as recombinant molecules in prokaryotic or eukaryotic host cells, such as bacteria, yeast, plant, animal (including insect) or human cell lines or in transgenic animals. Recombinant methods of producing a polypeptide through the introduction of a vector including nucleic acid encoding the polypeptide into a suitable host cell is well known in the art, such as is described in Sambrook et al., Molecular Cloning: A Laboratory Manual, 2d Ed, Vols 1 to 8, Cold Spring Harbor, NY (1989); M. W. Pennington and B. M. Dunn, Methods in Molecular Biology: Peptide Synthesis Protocols, Vol 35, Humana Press, Totawa, NJ (1994), contents of both of which are herein incorporated by reference.

The production of protein effector polypeptides at high levels in suitable host cells requires the assembly of the polynucleotides encoding such protein effector polypeptides into efficient transcriptional units together with suitable regulatory elements in a recombinant expression vector that can be propagated in various expression systems according to methods known to those skilled in the art. Efficient transcriptional regulatory elements could be derived from viruses having animal cells as their natural hosts or from the chromosomal DNA of animal cells. For example, promoter-enhancer combinations derived from the Simian Virus 40, adenovirus, BK polyoma virus, human cytomegalovirus, or the long terminal repeat of Rous sarcoma virus, or promoter-enhancer combinations including strongly constitutively transcribed genes in animal cells like beta-actin or GRP78 can be used. In order to achieve stable high levels of mRNA, the transcriptional unit should contain in its 3′-proximal part a DNA region encoding a transcriptional termination-polyadenylation sequence. Generally, this sequence can be derived from the Simian Virus 40 early transcriptional region, the rabbit beta-globin gene, or the human tissue plasminogen activator gene.

The vector is transfected into a suitable host cell line for expression of the protein effector polypeptide and/or the guide nucleic acid. Examples of cell lines that can be used to prepare the protein effector polypeptide and/or the guide nucleic acid described herein include, but are not limited to monkey COS-cells, mouse L-cells, mouse C127-cells, hamster BHK-21 cells, human embryonic kidney 293 cells, and hamster CHO-cells.

The expression vector encoding the protein effector polypeptide and/or the guide nucleic acid can be introduced in several different ways. For instance, the expression vectors can be created from vectors based on different animal viruses. Examples of these are vectors based on baculovirus, vaccinia virus, adenovirus, and preferably bovine papilloma virus.

The transcription units encoding the corresponding DNAs can also be introduced into animal cells together with another recombinant gene, which may function as a dominant selectable marker in these cells in order to facilitate the isolation of specific cell clones, which have integrated the recombinant DNA into their genome. Examples of this type of dominant selectable marker genes are Tn5 amino glycoside phosphotransferase, conferring resistance to geneticin (G418), hygromycin phosphotransferase, conferring resistance to hygromycin, and puromycin acetyl transferase, conferring resistance to puromycin. The recombinant expression vector encoding such a selectable marker can reside either on the same vector as the one encoding the cDNA of the desired protein, or it can be encoded on a separate vector which is simultaneously introduced and integrated to the genome of the host cell, frequently resulting in a tight physical linkage between the different transcription unit.

Other types of selectable marker genes, which can be used together with the cDNA of the desired protein are based on various transcription units encoding dihydrofolate reductase (dhfr). After introduction of this type of gene into cells lacking endogenous dhfr-activity, preferentially CHO-cells (DUKX-B11, DG-44) it will enable these to grow in media lacking nucleosides. An example of such a medium is Ham's F12 without hypoxanthine, thymidin, and glycine. These dhfr-genes can be introduced together with the Kazal-type serine protease inhibitors' cDNA transcriptional units into CHO-cells of the above type, either linked on the same vector or on different vectors, thus creating dhfr-positive cell lines producing recombinant protein.

If the above cell lines are grown in the presence of the cytotoxic dhfr-inhibitor methotrexate, new cell lines resistant to methotrexate will emerge. These cell lines may produce recombinant protein at an increased rate due to the amplified number of linked dhfr and the desired protein's transcriptional units. When propagating these cell lines in increasing concentrations of methotrexate (1-10000 nM), new cell lines can be obtained which produce the desired protein at a very high rate.

The above cell lines producing the desired protein effector polypeptide and/or guide nucleic acid can be grown on a large scale, either in suspension culture or on various solid supports. Examples of these supports are micro carriers based on dextran or collagen matrices, or solid supports in the form of hollow fibers or various ceramic materials. When grown in cell suspension culture or on micro carriers the culture of the above cell lines can be performed either as a batch culture or as a perfusion culture with continuous production of conditioned medium over extended periods of time. Thus, according to the present invention, the above cell lines are well suited for the development of an industrial process for the production of the desired protein effector polypeptide and/or the guide nucleic acid.

An example of such purification is the adsorption of the protein effector polypeptide to a monoclonal antibody or a binding peptide, which is immobilized on a solid support. After desorption, the protein can be further purified by a variety of chromatographic techniques based on the above properties.

Exemplary genera of yeast contemplated to be useful in the production of the protein effector polypeptide and/or described herein as hosts are Pichia (formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Genera include those selected from the group consisting of Saccharomyces, Schizosaccharomyces, Kluyveromyces, Pichia and Torulaspora. Examples of Saccharomyces spp. are S. cerevisiae, S. italicus and S. rouxii.

Suitable promoters for S. cerevisiae include those associated with the PGKI gene, GALI or GAL10 genes, CYCI, PHO5, TRPI, ADHI, ADH2, the genes for glyceral-dehyde-3-phosphate dehydrogenase, hexokinase, pyruvate decarboxylase, phos-phofructokinase, triose phosphate isomerase, phosphoglucose isomerase, glucokinase, alpha-mating factor pheromone, the PRBI, the GUT2, the GPDI promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (Maundrell K. 1990. Nmtl of fission yeast. A highly transcribed gene completely repressed by thiamine. J. Biol. Chem. 265:10857-10864) and the glucose repressible jbpl gene promoter as described by Hoffman and Winston (Hoffman C S and Winston F. 1990. Isolation and characterization of mutants constitutive for expression of the fbp1 gene of Schizosaccharomyces pombe. Genetics 124:807-816).

The transcription termination signal may be the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADHI gene is optionally used.

Exemplary expression systems for the production of the protein effector polypeptide and/or the guide nucleic acids described herein in bacteria include Bacillus subtilis, Bacillus brevis, Bacillus megaterium, Caulobacter crescentus, and, most importantly, Escherichia coli BL21 and E. coli K12 and their derivatives. Convenient promoters include but are not limited to trc promoter, tac promoter, lac promoter, lambda phage promoter p_{L}, the L-arabinose inducible araBAD promoter, the L-rhamnose inducible rhaP promoter, and the anhydrotetracycline-inducible tetA promoter/operator.

In some embodiments, the protein effector polypeptide or the polynucleotide encoding the protein effector polypeptide, further comprises a signal sequence and/or a leader sequence. A signal sequence (sometimes referred to as signal peptide, targeting signal, localization signal, localization sequence, or transit peptide) is a short “pre-peptide” (usually 16-30 amino acids long) present at the N-terminus (or occasionally non-classically at the C-terminus or internally) of most newly synthesized secretory proteins. The signal sequence facilitates translocation of the expressed polypeptide to which it is attached into the endoplasmic reticulum. Signal peptide typically comprises a positively charged n-region, a hydrophobic h-region, and a neutral, polar c-region. At the end of the signal sequence, there is typically a stretch of amino acids that is recognized and cleaved by a signal peptidase and therefore named the cleavage site. The signal sequence is normally cleaved off in the course of the secretion process.

In some embodiments, a polynucleotide encoding the protein effector polypeptide described herein can be fused to signal sequences which will direct the localization of the protein effector polypeptide to particular compartments of a prokaryotic cell and/or direct the secretion of a protein of the invention from a prokaryotic cell. For example, in E. coli, one may wish to direct the expression of the protein to the periplasmic space. Examples of signal sequences or proteins (or fragments thereof) to which the proteins of the invention may be fused in order to direct the expression of the polypeptide to the periplasmic space of bacteria include, but are not limited to, the pelB signal sequence, the maltose binding protein signal sequence, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (New England Biolabs).

The expression of the protein effector polypeptides from a polypeptide or vector encoding same can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the protein effector polypeptides can be modulated via a riboswitch, which can sense a small molecule like tetracycline. See, for example, Goldfless, Stephen J. et al. “Direct and specific chemical control of eukaryotic translation with a synthetic RNA-protein interaction,” Nucl. Acids Res., 40.9 (2012): e64-e64). Some exemplary methods and constructs for inducible CRISPR enzymes and inducible CRISPR systems amenable to the present invention are described, for example, in U.S. Pat. No. 8,871,445, US20160208243, and WO2016205764, each of which is incorporated herein by reference in its entirety.

### Nucleic Acid Modifications

One or more chemical or nucleic acid modifications can be applied to polynucleotides, e.g., ωRNAs described herein, guide nucleic acids described herein, polynucleotides encoding a protein effector polypeptide described herein, or polynucleotides encoding a guide nucleic acid described herein. Exemplary nucleic acid modifications include, but are not limited to, nucleobase modifications, sugar modifications, inter-sugar linkage modifications, conjugates (e.g., ligands), and any combinations thereof. Nucleic acid modifications also include unnatural, or degenerate nucleobases.

Exemplary modified nucleobases include, but are not limited to, inosine, xanthine, hypoxanthine, nebularine, isoguanosine, tubercidin, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

In some embodiments, a modified nucleobase can be selected from the group consisting of: inosine, xanthine, hypoxanthine, nebularine, isoguanosine, tubercidin, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N6-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N6-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N4-(acetyl)cytosine, 3-(3-amino-3-carboxypropyl)uracil, 5-ethynyl-2′-deoxyuridine, 2-(thio)uracil,5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N3-(methyl)uracil, 5-uracil (i.e., pseudouracil), 2-(thio)pseudouracil, 4-(thio)pseudouracil,2,4-(dithio) psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2-(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2-(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2-(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidin, isoguanosine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo) thymine, 2-pyridinone, 5-nitroindole, 3-nitropyrrole, 6-(aza)pyrimidine, 2-(amino)purine, 2,6-(diamino)purine, 5-substituted pyrimidines, N2-substituted purines, N6-substituted purines, 06-substituted purines, substituted 1,2,4-triazoles, and any O-alkylated or N-alkylated derivatives thereof.

In some embodiments, a nucleic acid modification can include a non-natural or modified nucleobase.

Exemplary sugar modified nucleotides include, but are not limited to, 2′-O-methyl(2′-OMe) nucleotides, 2′-fluoro (2′-F) nucleotides, 3′-fluoro nucleotides, 3′-OMe nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl(2′-MOE) nucleotides, 2′-O-allyl nucleotides, 2′-C-allyl ribose nucleotides, 2′-O-N-methylacetamido (2′-O-NMA) nucleotides, a 2′-O-dimethylaminoethoxyethyl(2′-O-DMAEOE) nucleotides, 2′-O-aminopropyl(2′-O-AP) nucleotides, 2′-F arabinose (2′-ara-F) nucleotides, threose (Threose nucleic acid, TNA) nucleotides, and acyclic nucleotides (e.g., peptide nucleic acid (PNA), unlocked nucleic acids (UNA), 2,3-dihydroxylpropyl (glycol nucleic acid, GNA)), and 2′-deoxy (2′-H); a modified internucleoside linkage; a non-natural or modified nucleobase; or a combination thereof.

In some embodiments, a sugar modified nucleotides can be a 2′-OMe nucleotide, 2′-F nucleotide, 2′-MOE nucleotide, BNA (e.g., LNA or ENA) nucleotide, UNA nucleotide, GNA nucleotide,

In some embodiments, a nucleic acid modification can include replacement or modification of an inter-sugar linkage., i.e., a modified internucleoside linkage. Exemplary inter-sugar linkage modifications include, but are not limited to, phosphotriesters, methylphosphonates, phosphoramidate, phosphorothioates, methylenemethylimino, thiodiester, thionocarbamate, siloxane, N,N′-dimethylhydrazine (—CH₂—N(CH₃)—N(CH₃)—), amide-3 (3′-CH₂—C(═O)—N(H)-5′) and amide-4 (3′-CH₂—N(H)—C(═O)-5′), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methylenecarbonylamino, methylenemethylimino (MMI, 3′-CH₂—N(CH₃)—O-5′), methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′, C3′-CH₂—NH—NH—C5′, 3′-NHP(O)(OCH₃)—O-5′ and 3′-NHP(O)(OCH₃)—O-5′).

Backbone modifications such as phosphorothioates modify the charge on the phosphate backbone and can aid in the delivery and nuclease resistance of the oligonucleotide (see, e.g., Eckstein, “Phosphorothioates, essential components of therapeutic oligonucleotides,” Nucl. Acid Ther., 24 (2014), pp. 374-387). Modifications of sugars, such as 2′-O-methyl(2′-OMe), 2′-F, and locked nucleic acid (LNA), can enhance both base pairing and nuclease resistance (see, e.g., Allerson et al., “Fully 2′-modified oligonucleotide duplexes with improved in vitro potency and stability compared to unmodified small interfering RNA,” J Med. Chem., 48.4 (2005): 901-904). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet. 2012 Aug. 20; 3:154). Additionally, the guide nucleic acid is amenable to both 5′ and 3′ end conjugations with a variety of functional moieties including, but not limited to, targeting ligands, fluorescent dyes, polyethylene glycol, or proteins.

In some embodiments of any one of the aspects described herein, each modified internucleoside linkage can be selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, 5′->5′ internucleoside linkages, and imidophosphoramidate (“imidp”) linkage

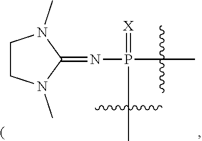

where X is O or S, preferably X is O).

A summary of chemical modifications amenable to the ωRNAs and guide nucleic acids described herein can be found, e.g., in Kelley et al., “Versatility of chemically synthesized guide RNAs for CRISPR-Cas9 genome editing,” J. Biotechnol. 2016 Sep. 10; 233:74-83; WO 2016205764; and U.S. Pat. No. 8,795,965 B2; each which is incorporated by reference in its entirety.

### Target Adjacent Motif

As used herein, the term “target adjacent motif” or “TAM” refers to a nucleotide sequence adjacent to a target sequence to which a complex comprising a protein effector polypeptide and a nucleic acid guide binds. In some embodiments, a TAM is required for binding of a protein effector polypeptide and a nucleic acid guide to a target nucleic acid. It is noted that the term TAM is used interchangeably with the term “protospacer adjacent motif” or “PAM” herein. As used herein, the term “adjacent” includes instances in which a nucleic acid guide of the complex specifically binds, interacts, or associates with a target sequence that is immediately adjacent to a TAM. In such instances, there are no nucleotides between the target sequence and the TAM. The term “adjacent” also includes instances in which there are a small number (e.g., 1, 2, 3, 4, or 5) of nucleotides between the target sequence, to which the targeting moiety binds, and the TAM.

In some embodiments, the cleavage site is distant from the TAM, e.g., the cleavage occurs after the nth nucleotide on the non-target strand and after the nucleotide on the targeted strand. In some embodiments, the cleavage site occurs after an identified nucleotide (counted from the TAM) on the non-target strand and after the further identified nucleotide (counted from the TAM) on the targeted strand.

In some embodiments, a protein effector polypeptide described herein exhibits an increased activity on a target sequence comprising a TAM sequence selected from the group consisting of RTAAA, ATRAA, ATRMM, ATRA, ATRRA, ATARA, ATRRAC, ATAAAC, ATARM, ATRAM, ATRRM, ATRMA, ATANA, ATRR, ATR, ATGAC, ATAA, ATAR, ATGMC, ATGAA, and ATRAR (where R is purine (e.g., A or G) and M is A) at its 3′ end as compared to a wild-type transposon family encoded nuclease, such as wild-type IscB. Accordingly, in some embodiments, the TAM comprises a nucleotide sequence selected from the group consisting of RTAAA, ATRAA, ATRMM, ATRA, ATRRA, ATARA, ATRRAC, ATAAAC, ATARM, ATRAM, ATRRM, ATRMA, ATANA, ATRR, ATR, ATGAC, ATAA, ATAR, ATGMC, ATGAA, and ATRAR, where R is purine (e.g., A or G) and M is A or C.

### Target Nucleic Acids

In some embodiments, modifying the target nucleic acid comprises binding, nicking, cleaving, or marking the target nucleic acid. The target nucleic acid can be DNA or RNA. In some embodiments, the target nucleic acid can be a DNA sequence. In some embodiments, the target nucleic acid can be an RNA sequence. In some embodiments, the target nucleic acid can be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target nucleic acid can be a sequence within a RNA molecule selected from the group consisting of mRNA, pre-mRNA, and rRNA. In some preferred embodiments, the target nucleic acid can be a sequence within a RNA molecule selected from the group consisting of ncRNA, and lncRNA. In some more preferred embodiments, the target nucleic acid can be a sequence within an mRNA molecule or a pre-mRNA molecule. In some embodiments, the target nucleic acid is genomic DNA, episomal DNA, viral DNA, viral RNA, or bacterial DNA.

In some embodiment, the target nucleic acid sequence comprises a sequence associated with a disease or disorder.

In some embodiments, the target nucleic acid is a double-stranded DNA. In some embodiments, the target nucleic acid is a single-stranded DNA. In some embodiments, the target nucleic acid is a single-stranded RNA. In some embodiments, the target nucleic acid is a double-stranded RNA. In some embodiments, the sequence-specificity requires a complete match of the guide sequence in the guide nucleic acid to the target nucleic acid. In some other embodiments, the sequence specificity requires a partial (contiguous or non-contiguous) match of the guide sequence in the guide nucleic acid to the target nucleic acid.

The target nucleic acid can be in vitro. In some embodiments, the target nucleic acid is within a cell. For example, the target nucleic acid is within a prokaryotic cell, a bacterial cell, a eukaryotic cell, a fungal cell, a plant cell, an animal cell, a mammalian cell, a rodent cell, a primate cell, or a human cell. In some embodiments, the target nucleic acid is located in the nucleus or cytoplasm of a cell.

If the target nucleic acid is double-stranded, the strand of the double-stranded target nucleic acid that is complementary to and hybridizes with the guide nucleic acid can be referred to as the complementary strand. The strand of the double-stranded target nucleic acid that is complementary to the complementary strand, and therefore not complementary to the guide nucleic acid can be referred to as the noncomplementary strand.

In some embodiments of any one of the aspects described herein, the target nucleic acid sequence is in a cell. For example, the target nucleic acid sequence is in a mammalian cell, such as human or non-human mammalian cell.

In some embodiments, the target nucleic acid sequence is in the genome of an organism. For example, the target nucleic acid sequence is in the genome of a prokaryote, e.g., a bacterium. In some embodiments, the target nucleic acid sequence is in the genome of a eukaryote (including mammal including human) or a non-human eukaryote or a non-human animal or a non-human mammal. For example, the target nucleic acid sequence is in the genome of an organism or subject that is a non-human animal. In some embodiments, the organism or subject can be an arthropod, for example, an insect, or may be a nematode. In some embodiments, the target nucleic acid sequence is in the genome of a plant. In some embodiments, the target nucleic acid sequence is in the genome of a mammal or a non-human mammal. A non-human mammal can be for example a rodent (preferably a mouse or a rat), an ungulate, or a primate. In some embodiments, the organism or subject is algae, including microalgae, or is a fungus.

In some embodiments, the 3′-end of the target nucleic acid sequence is directly adjacent to a sequence selected from the group consisting of AAA, AAC, AAG, AAT, CAA, CAC, CAG, CAT, GAA, GAC, GAG, GAT, TAA, TAC, TAG, TAT, ACA, ACC, ACG, ACT, CCA, CCC, CCG, CCT, GCA, GCC, GCG, GCT, TCA, TCC, TCG, TCT, AGA, AGC, AGT, CGA, CGC, CGT, GGA, GGC, GGT, TGA, TGC, TGT, ATA, ATC, ATG, ATT, CTA, CTC, CTG, CTT, GTA, GTC, GTG, GTT, TTA, TTC, TTG, and TTT. For example, the 3′-end of the target nucleic acid sequence is directly adjacent to the sequence AAA, GAA, CAA, or TAA. In another example, the 3′-end of the target nucleic acid sequence is directly adjacent to the sequence AAC, GAC, CAC, or TAC. In yet another example, the 3′-end of the target nucleic acid sequence is directly adjacent to the sequence AAT, GAT, CAT, or TAT.

### Compositions

A variety of composition can be prepared for use in the methods described herein, depending upon the intended use of the composition. Accordingly, in another aspect, provided herein is a composition, e.g., a pharmaceutical composition comprising one or more described herein (e.g., including, but not limited to, the protein effector polypeptides (or polynucleotides encoding same), fusion proteins (or polynucleotides encoding same), guide nucleic acids (or polynucleotides encoding same), and complexes comprising same).

The compositions described herein can optionally comprise additional components. For example, the composition can further comprise a source of magnesium. In some embodiments, the composition further comprises Mg²⁺.

In some embodiments, a composition described herein further comprises a single- or double-stranded DNA repair template comprising from 5′ to 3′: a first homology arm comprising a sequence of at least 20 nucleotides 5′ to the target deoxyribonucleic acid sequence, a synthetic DNA sequence of at least 10 nucleotides, and a second homology arm comprising a sequence of at least 20 nucleotides 3′ to the target sequence. In some embodiments, the first or second homology arm comprises a sequence of at least 40, 80, 120, 150, 200, 300, 500, or 1,000 nucleotides.

In some embodiments, the composition is a pharmaceutical composition. These pharmaceutically acceptable compositions comprise one or more of the various components described herein (e.g., including, but not limited to, the protein effector polypeptides (or polynucleotides encoding same), fusion proteins (or polynucleotides encoding same), guide nucleic acids (or polynucleotides encoding same), and complexes comprising same), formulated together with one or more pharmaceutically acceptable carriers (additives) and/or diluents. As described in detail below, the pharmaceutical compositions described herein can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24:199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used here, the term “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the composition is in form of a liposome. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. In some embodiments, the liposomes are also specifically targeted, e.g., to direct the protein effector polypeptides (or polynucleotides encoding same), fusion proteins (or polynucleotides encoding same), guide nucleic acids (or polynucleotides encoding same), and/or complexes comprising same to particular cell types.

### LNPs

In some embodiments, the composition is in form of lipid nanoparticles (NLPs). For example, the composition comprises one or more of the various components described herein (e.g., including, but not limited to, the protein effector polypeptides (or polynucleotides encoding same), fusion proteins (or polynucleotides encoding same), guide nucleic acids (or polynucleotides encoding same), and complexes comprising same) and a cationic lipid. Exemplary cationic lipids include, but are not limited to, N-[1-(2,3-dioleyloxy) propyl-N,N,N-trimethylammonium chloride (DOTMA); N-[1-(2,3-dioleoyloxy) propyl-N,N,N-trimethylammonium chloride (DOTAP); 1,2-dioleoyl-sn-glycero-3-ethylphosphocholine (DOEPC); 1,2-dilauroyl-sn-glycero-3-ethylphosphocholine (DLEPC); 1,2-dimyristoyl-sn-glycero-3-ethylphosphocholine (DMEPC); 1,2-dimyristoleoyl-sn-glycero-3-ethylphosphocholine (14:1), N1-[2-((1 S)-1-[(3-aminopropyl)amino]-4-[di (3-amino-propyl) aminolbutylcarboxamidoiethyl1-3,4-di [oleyloxy]-benzamide (MVL5); Dioctadecylamido-glycylspermine (DOGS); 3b-[N-(N′,N′-dimethylaminoethyl)carb amoyl]cholesterol (DC-Chol); Dioctadecyldimethylammonium Bromide (DDAB); a Saint lipid (e.g., SAINT-2, N-methy 1-4-(dioleyl)methylpyridinium); 1,2-dimyristyloxypropy 1-3-dimethylhydroxyethylammonium bromide (DMRIE); 1,2-dioleoy 1-3-dimethyl-hydroxyethyl ammonium bromide (DORIE); 1,2-dioleoyloxypropy 1-3-dimethylhydroxyethyl ammonium chloride (DORI); Di-alkylated Amino Acid (DILA2) (e.g., C18: 1-norArg-C16); Dioleyldimethylammonium chloride (DODAC); 1-p almitoy 1-2-oleoyl-sn-glycero-3-ethylpho sphocholine (POEPC); and 1,2-dimyristoleo yl-sn-glycero-3-ethylphosphocholine (MOEPC). In some variations, the condensing agent, e.g. a cationic lipid, is a lipid such as, e.g., Dioctadecy ldimethylammonium bromide (DDAB), 1,2-dilinoleyloxy-3-dimethylaminopropane (DLinDMA), 2,2-dilinoley 1-4-(2dimethylaminoethyl)-[1,31-dioxolane (DLin-KC2-DMA), heptatriaconta-6,9,28,31-tetraen-19-yl-4-(dimethylamino)butanoate (DLin-MC3-DMA), 1,2-Dioleoyloxy-3-dimethylaminopropane (DODAP), 1,2-Dioleyloxy-3-dimethylaminopropane (DODMA), Morpholinocholesterol (Mo-CHOL), (R)-5-(dimethylamino)pentane-1,2-diyl dioleate hydrochloride (DODAPen-C1), (R)-5-guanidinopentane-1,2-diy 1 dioleate hydrochloride (DOPen-G), and (R)-N,N,N-trimethyl-4,5-bis(oleoyloxy) pentan-1-aminium chloride (DOTAPen). The cationic lipid can comprise 20-90% (mol) of the total lipid present in the lipid nanoparticle. For example, cationic lipid molar content can be 20-70% (mol), 30-60% (mol) or 40-50% (mol) of the total lipid present in the lipid nanoparticle. In some embodiments, cationic lipid comprises from about 50 mol % to about 90 mol % of the total lipid present in the lipid nanoparticle.

In some embodiments, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity. The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of cationic lipid to the neutral lipid ranges from about 2:1 to about 8:1. Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, lysophosphatidylcholine, dilinoleoylphosphatidylcholine, or mixtures thereof. It is to be understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Other examples of non-cationic lipids suitable for use in the lipid nanoparticles include nonphosphorous lipids such as, e.g., stearylamine, dodecylamine, hexadecylamine, acetyl palmitate, glycerolricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In some embodiments, the non-cationic lipid is a phospholipid. In some embodiments, the non-cationic lipid is selected from DSPC, DPPC, DMPC, DLPE, DMPE, DPHyPe, DOPC, POPC, DOPE, and SM. In some preferred embodiments, the non-cationic lipid is DSPC.

In some embodiments, the lipid nanoparticle can further comprise a component, such as a sterol, to provide membrane integrity. The component providing membrane integrity, such as a sterol, can comprise 0-50% (mol) of the total lipid present in the lipid nanoparticle. In some embodiments, such a component is 20-50% (mol) 30-40% (mol) of the total lipid content of the lipid nanoparticle. Generally, the component used for providing membrane integrity is non-fusogenic, i.e., a component that does not or substantially does not fuse with a membrane or, if does fuse with a membrane, does not destabilize the membrane. Generally, the component used in the nanoparticles of the invention for providing membrane integrity does not have, or has very little, fusogenic activity at any pH.

One exemplary sterol that can be used in the lipid nanoparticle is cholesterol and derivatives thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In some embodiments, the cholesterol derivative is a polar analogue such as cholesteryl-(4′-hydroxy)-butyl ether. In some embodiments, cholesterol derivative is cholestryl hemisuccinate (CHEMS).

In some embodiments, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, conjugated lipid molecule is a PEG-lipid conjugate, for example, a (methoxy polyethylene glycol)-conjugated lipid. The PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di (tetradecanoyloxy) propyl-1-O-(w-methoxy (polyethoxy)ethyl)butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, US2018/0028664, US2015/0376115 and US2016/0376224, contents of all which are incorporated herein by reference in their entirety.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3 [beta]-oxy) carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy (polyethylene glycol)-2000]. In some embodiments, the PEG-lipid is selected from the group consisting N-(Carbonyl-methoxypolyethyleneglycoln)-1,2-dimyristoyl-sn-glycero-3-phosphoethanolamine (DMPE-PEGₙ, where n is 350, 500, 750, 1000 or 2000), N-(Carbonyl-methoxypolyethyleneglycolₙ)-1,2-distearoyl-sn-glycero-3-phosphoethanolamine (DSPE-PEGₙ, where n is 350, 500, 750, 1000 or 2000), DSPE-polyglycelin-cyclohexyl-carboxylic acid, DSPE-polyglycelin-2-methylglutar-carboxylic acid, polyethylene glycol-dimyristolglycerol (PEG-DMG), polyethylene glycol-distearoyl glycerol (PEG-DSG), or N-octanoyl-sphingosine-1-{succinyl[methoxy(polyethylene glycol) 200011 (C8 PEG2000 Ceramide). In some examples of DMPE-PEGₙ, where n is 350, 500, 750, 1000 or 2000, the PEG-lipid is N-(Carbonyl-methoxypolyethyleneglycol 2000)-1,2-dimyristoyl-sn-glycero-3-phosphoethanolamine (DMPE-PEG 2,000). In some examples of DSPE-PEG. where n is 350, 500, 750, 1000 or 2000, the PEG-lipid is N-(Carbonyl-methoxypolyethyleneglycol 2000)-1,2-distearoyl-sn-glycero-3-phosphoethanolamine (DSPE-PEG 2,000). In some preferred embodiments, the PEG-lipid is PEG-DMG.

Lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid.

Generally, the lipid nanoparticles have a mean diameter selected to provide an intended therapeutic effect. Accordingly, in some embodiments, the lipid nanoparticle has a mean diameter from about 30 nm to about 150 nm, more typically from about 50 nm to about 150 nm, more typically about 60 nm to about 130 nm, more typically about 70 nm to about 110 nm, most typically about 85 nm to about 105 nm, and preferably about 100 nm. In some embodiments, the lipid particles that larger in relative size to common nanoparticles and about 150 to 250 nm in size.

Depending on the intended use of the lipid particles, the proportions of the components can be varied and the delivery efficiency of a particular formulation can be measured using, for example, an endosomal release parameter (ERP) assay.

### Protein Nanoparticles

In some embodiments, the composition can be inform of protein nanoparticles. Non-limiting examples of protein nanoparticles include ferritin nanoparticles (see, e.g., Zhang, Y. Int. J. Mol., 12:5406-5421, 2011, incorporated by reference herein), encapsulin nanoparticles (see, e.g., Sutter et al., Nature Struct, and Mol. Biol., 15:939-947, 2008, incorporated by reference herein), Sulfur Oxygenase Reductase (SOR) nanoparticles (see, e.g., Urich et al., Science, 311:996-1000, 2006, incorporated by reference herein), lumazine synthase nanoparticles (see, e.g., Zhang et al., J. Mol. Biol., 306:1099-1114, 2001) or pyruvate dehydrogenase nanoparticles (see, e.g., Izard et al., PNAS 96:1240-1245, 1999, incorporated by reference herein). Ferritin, encapsulin, SOR, lumazine synthase, and pyruvate dehydrogenase are monomeric proteins that self-assemble into a globular protein complexes that in some cases consists of 24, 60, 24, 60, and 60 protein subunits, respectively.

### Other Exemplary Compositions

In some embodiments, the composition is in form of lipoplexes/polyplexes. Lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2p (e.g., forming DNA/Ca²⁺ microcomplexes), polyethylenimine (PEI) (e.g., branched PEI), and poly(L-lysine) (PLL).

In some embodiments, the composition is in form DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136 (42): 14722-5; and Sun W et al, Angew Chem Int Ed Engl. 2015 Oct. 5; 54 (41): 12029-33. DNA nanoclew may have a palindromic sequences to be partially complementary to the guide RNA within the IscB polypeptide nuclease: hRNA ribonucleoprotein complex. A DNA nanoclew may be coated, e.g., coated with PEI to induce endosomal escape.

In some embodiments, the composition can comprise one or more of the various components described herein (e.g., including, but not limited to, the protein effector polypeptides (or polynucleotides encoding same), fusion proteins (or polynucleotides encoding same), guide nucleic acids (or polynucleotides encoding same), and complexes comprising same) complexed with gold nanoparticles. Gold nanoparticles may be coated, e.g., coated in a silicate and an endosomal disruptive polymer, PAsp (DET). Examples of gold nanoparticles include AURASENSE Therapeutics' Spherical Nucleic Acid (SNA™) constructs, and those described in Mout R, et al. (2017). ACS Nano 11:2452-8; Lee K, et al. (2017). Nat Biomed Eng 1:889-901.

In some embodiments, the composition further comprises iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

In some embodiments, the composition is in form of polymer-based particles (e.g., nanoparticles). In one embodiment, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In one embodiment, the polymer-based particles may comprise alkylated and carboxyalkylated branched polyethylenimine. In some examples, the polymer-based particles are VIROMER, e.g., VIROMERRNAi, VIROMERRED, VIROMER mRNA. Example methods of delivering the systems and compositions herein include those described in Bawage S S et al., Synthetic mRNA expressed Cas13a mitigates RNA virus infections, biorxiv.org/content/10.1101/370460vl.full doi: doi.org/10.1101/370460, Viromer® RED, a powerful tool for transfection of keratinocytes. doi: 10.13140/RG.2.2.16993.61281, Viromer® Transfection-Factbook 2018: technology, product overview, users' data., doi: 10.13140/RG.2.2.23912.16642.

In some embodiments, the composition is in form of lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In one embodiment, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

In some embodiments, the composition comprises inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

In some embodiments, the composition is in form of exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267 (1): 9-21; El-Andaloussi S, et al., Nat Protoc. 2012 December; 7 (12): 2112-26; Uno Y, et al., Hum Gene Ther. 2011 June; 22 (6): 711-9; Zou W, et al., Hum Gene Ther. 2011 April; 22 (4): 465-75. In some examples, the exosome may form a complex (e.g., by binding directly or indirectly) to one or more of the various components described herein (e.g., including, but not limited to, the protein effector polypeptides (or polynucleotides encoding same), fusion proteins (or polynucleotides encoding same), guide nucleic acids (or polynucleotides encoding same), and complexes comprising same). In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

### Uses

Engineered, non-natural DNA and RNA targeting complexes described herein can be used for various applications, such as, for example, nucleic acid editing (e.g., gene editing), binding to a nucleic acid molecule (e.g., sequence-specific binding). The engineered, non-natural DNA and RNA targeting complexes described herein can be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

In some embodiments, the engineered, non-naturally occurring DNA and RNA targeting system or composition described herein can be used in multiplexed error-robust fluorescence in situ hybridization (MERFISH). These methods are described in, e.g., Chen et at., “Spatially resolved, highly multiplexed RNA profiling in single cells,” Science, 2015 Apr. 24; 348 (6233): aaa6090, which is incorporated herein by reference in its entirety.

The engineered, non-naturally occurring DNA and RNA targeting system or composition described herein can be used as non-specific RNase, e.g., to cleave and/or degrade RNA molecules present near a target nucleic acid. For example, a complex comprised of a protein effector polypeptide and a guide nucleic acid is activated upon binding to a target nucleic acid. Activation induces a conformational change, which results in the activated complex acting as a non-specific RNase, cleaving and/or degrading nearby RNA molecules (e.g., ssRNA or dsRNA molecules), i.e., “collateral” effects.

The engineered, non-naturally occurring DNA and RNA targeting system or composition described herein can be used as non-specific DNase, e.g., to cleave and/or degrade DNA molecules present near a target nucleic acid. For example, a complex comprised of a protein effector polypeptide and a guide nucleic acid is activated upon binding to a target nucleic acid. Activation induces a conformational change, which results in the activated complex acting as a non-specific DNase, cleaving and/or degrading nearby DNA molecules (e.g., ssDNA or dsDNA molecules), i.e., “collateral” effects.

The engineered, non-naturally occurring DNA and RNA targeting system or composition described herein can be used for collateral-free target recognition. For example, a complex comprised of a protein effector polypeptide and a guide nucleic acid does not exhibit collateral nuclease activity subsequent to target recognition. This “collateral-free” embodiment can comprise wild-type or engineered protein effector polypeptides.

The engineered, non-naturally occurring DNA and RNA targeting system or composition described herein can be used for TAM/PFS-Independent Targeting. For example, a complex comprised of a protein effector polypeptide and a guide nucleic acid recognizes and cleaves the target nucleic acid without any additional requirements adjacent to or flanking the protospacer (i.e., target adjacent motif “TAM” or protospacer flanking sequence “PFS” requirements).

### Methods for Modifying Target Nucleic Sequences

In another aspect, provided herein is a method for modifying a target nucleic acid sequence. The method comprises contacting a target nucleic acid sequence with a protein effector polypeptide (or a fusion protein comprising same) and a guide nucleic acid (e.g., gRNA or ωRNA). For example, contacting the target nucleic acid sequence with an engineered, non-natural DNA and RNA targeting complex described herein.

In some embodiments, the target nucleic acid sequence is in a cell and the method comprises administering to the cell a protein effector polypeptide (or a polynucleotide encoding same) and a guide nucleic acid (or a polynucleotide encoding same).

In some embodiments, a target nucleic acid modification can include correcting a point mutation in the target sequence. For example, the target sequence comprises a point mutation associated with a disease or disorder. Such a point mutation can be located between about 10 to about 20 nucleotides upstream of the TAM in the target sequence. In some embodiments, the point mutation can be located 10, 11, 12, 13, 14, 15, 16, 17, 18, 19 or 20 nucleotides upstream of the TAM in the target sequence. Without wishing to be bound by a theory, correcting a point mutation results in a sequence that is not associated with a disease or disorder. The point mutation can be located in the strand of the target nucleic acid that is complementary to the guide sequence. Alternatively, the point mutation can be located in the strand of the target nucleic acid that is not complementary to the guide sequence.

In some embodiments, the target nucleic acid modification comprises a T→C or A→G point mutation associated with a disease or disorder, and modification of the target sequence comprises deamination of the mutant C or G base. It is noted that the T→C or A→G can be located between about 10 to about 20 nucleotides upstream of the TAM in the target sequence. In some embodiments, the T→C or A→G point mutation can be located 10, 11, 12, 13, 14, 15, 16, 17, 18, 19 or 20 nucleotides upstream of the TAM in the target sequence. In some embodiments, deamination of the mutant C or G base result in a sequence that is not associated with a disease or disorder. The T→C point mutation can be located in the strand of the target nucleic acid that is complementary to the guide sequence. The A→G point mutation can located in the strand of the target nucleic acid that is not complementary to the guide sequence. Some exemplary disease or disorders associated with T→C or A→G point mutation include, but are not limited to, cystic fibrosis, phenylketonuria, epidermolytic hyperkeratosis (EHK), Charcot-Marie-Toot disease type 4J, neuroblastoma (NB), von Willebrand disease (vWD), myotonia congenital, hereditary renal amyloidosis, dilated cardiomyopathy (DCM), hereditary lymphedema, familial Alzheimer's disease, Prion disease, chronic infantile neurologic cutaneous articular syndrome (CINCA), desmin-related myopathy (DRM), or a neoplastic disease associated with a mutant PI3KCA protein

In some embodiments, a target nucleic acid modification can include the introduction, deletion, or substitution of 1-75 nucleotides of the target sequence. For example, the modification can include the introduction, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides in the target sequence. The modification can include the introduction, deletion, or substitution of at least 5, 10, 11, 12, 13, 14, 15, 16, 17, 18, 19, 20, 21, 22, 23, 24, 25, 26, 27, 28, 29, 30, 35, 40, 45, 50, or 75 nucleotides. In some embodiments, the modification can include the introduction, deletion, or substitution of at least 20, 21, 22, 23, 24, 25, 26, 27, 28, 29, 30, 35, 40, 45, 50, or 75 nucleotides in the target sequence. In some embodiments, the modification can include the introduction, deletion, or substitution of at least 40, 45, 50, 75, 100, 200, 300, 400 or 500 nucleotides in the target sequence. In some embodiments, the modification can include the introduction, deletion, or substitution of at least 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 nucleotides in the target sequence.

The modifications can include Non-Homologous End-Joining (NHEJ). Thus, in one embodiment, modification of a target sequence by a complex, composition, system, or a component thereof described herein, such as a disease associated sequence, can include NHEJ. In some embodiments, promotion of this repair pathway by a complex, composition, system, or a component thereof described herein can be used to target gene or polynucleotide specific knockouts and/or knock-ins. In some embodiments, promotion of this repair pathway by a complex, composition, system, or a component thereof described herein can be used to generate NHEJ-mediated indels. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. The indel can range in size from 1-50 or more base pairs. In some embodiments, the indel can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences.

In some embodiments, NHEJ can be used in the method to delete small sequence motifs. In some embodiments, NHEJ can be used in the method to generate NHEJ-mediate indels that can be targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp). In some embodiments, a guide nucleic acid (e.g., gRNA or ωRNA) can be configured to position one double-strand break in close proximity to a nucleotide of the target position. In some embodiments, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position). In some embodiments, two guide RNAs may be configured to position two single-strand breaks to provide for NHEJ repair a nucleotide of the target position.

In some embodiments, the DNA and RNA targeting complex or components thereof can be administered to a cell by physical delivery methods. Exemplary physical methods include, but are not limited to, microinjection, electroporation, and hydrodynamic delivery. Both nucleic acid and proteins can be delivered using such methods. For example, one or more protein components can be prepared in vitro, isolated, (refolded, purified if needed), and introduced to cells.

In some embodiments, the DNA and RNA targeting complex or components thereof are microinjected directly to cells. Microinjection can achieve high efficiency, e.g., above 90% or about 100%. The microinjection can be performed using a microscope and a needle (e.g., with 0.5-5.0 μm in diameter) to pierce a cell membrane and deliver the DNA and RNA targeting complex or components thereof directly to a target site within the cell. Microinjection can be used for in vitro and ex vivo delivery. Plasmids comprising coding sequences for one or more protein components and/or guide nucleic acids and/or mRNAs may be microinjected. In some cases, microinjection can be used i) to deliver DNA directly to a cell nucleus, and/or ii) to deliver mRNA (e.g., in vitro transcribed) to a cell nucleus or cytoplasm. In certain examples, microinjection may be used to deliver guide nucleic acids directly to the nucleus and mRNA to the cytoplasm, e.g., facilitating translation and shuttling of one or more protein components to the nucleus. Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell.

In some embodiments, the DNA and RNA targeting complex or components thereof can be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery. Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

In some embodiments, the DNA and RNA targeting complex or components thereof may be delivered using hydrodynamic delivery, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

In some embodiments, polynucleotide(s) encoding one or more components of the DNA and RNA targeting complex may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Base Editing

The present disclosure also provides for base editing systems. In general, such a system (base-editing system) comprises a deaminase (e.g., an adenosine deaminase or cytidine deaminase), or factional fragment thereof associated, (e.g., fused or linked) with an protein effector polypeptide described herein. For example, the base-editing system comprises a fusion protein described herein, where the functional domain is a deaminase, e.g., a cytidine or adenosine deaminase. In some embodiments, the base-editing system comprises a catalytically inactive protein effector polypeptide associated (e.g., fused or linked) with a deaminase, and guide nucleic acid (e.g. ωRNA) capable of forming a complex with the protein effector polypeptide and directing site-specific binding of the complex to a target sequence of a target.

In some embodiments, the base-editing systems can comprise an intein-mediated trans-splicing system that enables in vivo delivery of a base editor, e.g., a split-intein cytidine base editors (CBE) or adenine base editor (ABE) engineered to trans-splice. Examples of the such base editing systems include those described in Colin K. W. Lim et al., Treatment of a Mouse Model of ALS by In Vivo Base Editing, Mol Ther. 2020 Jan. 14. pii: S1525-0016 (20) 30011-3. doi: 10.1016/j.ymthe.2020.01.005; and Jonathan M. Levy et al., Cytosine and adenine base editing of the brain, liver, retina, heart and skeletal muscle of mice via adeno-associated viruses, Nature Biomedical Engineering volume 4, pages97-1 10 (2020), which are incorporated by reference herein in their entireties. Examples of base editing systems include those described in International Patent Publication Nos. WO 2019/071048 (e.g. paragraphs [0933]-[0938]), WO 2019/084063 (e.g., paragraphs [0173]-[0186], [0323]-[0475], [0893]-[1094]), WO 2019/126716 (e.g., paragraphs [0290]-[0425], [1077]-[1084], WO 2019/126709 (e.g., paragraphs [0294]-[0453]), WO 2019/126762 (e.g., paragraphs [0309]-[0438]), WO 2019/126774 (e.g., paragraphs [0511]-[0670]), Cox D B T, et al., RNA editing with CRISPR-Cas13, Science. 2017 Nov. 24; 358 (6366): 1019-1027; Abudayyeh 00, et al., A cytosine deaminase for programmable single-base RNA editing, Science 26 Jul. 2019: Vol. 365, Issue 6451, pp. 382-386; Gaudelli N M et al., Programmable base editing of A»T to G»C in genomic DNA without DNA cleavage, Nature volume 551, pages 464-471 (23 Nov. 2017); Komor A C, et al., Programmable editing of a target base in genomic DNA without double-stranded DNA cleavage. Nature. 2016 May 19; 533 (7603): 420-4; Jordan L. Doman et al., Evaluation and minimization of Cas9-independent off-target DNA editing by cytosine base editors, Nat Biotechnol (2020). doi.org/10.1038/s41587-020-0414-6; and Richter M F et al., Phage-assisted evolution of an adenine base editor with improved Cas domain compatibility and activity, Nat Biotechnol (2020). doi.org/10.1038/s41587-020-0453-z, which are incorporated by reference herein in their entireties and can be used to adapt to protein effector polypeptides described herein.

### Prime Editing

The present disclosure also provides for prime editing systems. In general, such a system (prime-editing system) comprises a reverse transcriptase, or factional fragment thereof associated, (e.g., fused or linked) with an protein effector polypeptide described herein. For example, the prime-editing system comprises a fusion protein described herein, where the functional domain is a reverse transcriptase or a functional fragment thereof e.g., a cytidine or adenosine deaminase. In some embodiments, the prime-editing system comprises a catalytically inactive protein effector polypeptide described herein, a reverse transcriptase associated with or otherwise capable of forming a complex with the protein effector polypeptide, and guide nucleic acid (e.g., ωRNA) capable of forming a complex with the protein effector polypeptide and directing site-specific binding of the complex to a target sequence of a target sequence, optionally, the guide nucleic acid (e.g., ωRNA) further comprising a donor sequence for insertion into the target sequence. In some embodiments, the catalytically inactive protein effector polypeptide is a nickase, e.g., a DNA nickase. In some embodiments, the prime-editing system comprises a catalytically inactive protein effector polypeptide described herein, a reverse transcriptase associated with or otherwise capable of forming a complex with the protein effector polypeptide, and guide nucleic acid (e.g., ωRNA) capable of forming a complex with the protein effector polypeptide, and a where the guide nucleic acid comprises: a guide sequence capable of directing site-specific binding of the complex to a target sequence of a target sequence; a 3′ binding site region capable of binding to a cleaved upstream strand of the target sequence; and a RT template sequence encoding an extended sequence, wherein the extended sequence comprises a variant region and a 3′ homologous sequence capable of hybridization to the downstream cleaved strand of the target sequence.

The prime-editing system described herein can be used to prime-edit a single nucleotide on a target sequence (e.g., DNA). Alternatively, or additionally, the prime-editing system described herein can be used to prime-edit at least 2, at least 3, at least 4, at least 5, at least 6, at least 7, at least 8, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 200, at least 300, at least 400, at least 500, at least 600, at least 700, at least 800, at least 900, or at least 1000 nucleotides on a target sequence.

In some embodiments, the prime-editing system can be used to create a longer 3′ region (e.g., 20 nucleotides). In such embodiments, the prime-editing system comprises a protein effector polypeptide described herein with nickase activity, a reverse transcriptase domain, and a DNA polymerase, and guide nucleic acid comprising a binding sequence capable of hybridizing to the target sequence and an editing sequence. The generated region can be further extended on a DNA template as described herein. The latter can allow generation of a target-independent sequence, compatible with a generic donor sequence. Without wishing to be bound by a theory, the protein effector polypeptide is capable of generating a first cleavage of in the target sequence and a second cleavage outside the target sequence on the target polynucleotide. In some variations, a second protein effector polypeptide-mediated cleavage in vicinity to the target site may be made, which may enable more efficient invasion of the extended DNA.

In some embodiments, the prime-editing system further comprises a donor template.

In some embodiments, the prime-editing system further comprises a recombinase. The recombinase is connected to or otherwise capable of forming a complex with the effector protein polypeptide. In some embodiments, the complex is capable of inserting a recombination site in the DNA loci of interest by extension of RT templates that encode for the recombination site on the 3′ extension of the guide sequences by the reverse transcriptase. In some embodiments, a donor template comprising a compatible recombination site is provided that can recombine unidirectionally with the inserted recombination site when a recombinase specific for the recombination site is also provided. In some embodiments, donor template is a plasmid comprising the complementary recombination site and any sequence for insertion at the DNA loci of interest. In some embodiments, the recombinase is connected to or capable of forming a complex with the protein effector polypeptide, such that all of the enzymatic proteins are brought into contact at the loci of interest. In some embodiments, the recombinase is codon optimized for eukaryotic cells (described further herein). In some embodiments, the recombinase includes a NLS (described further herein). In some embodiments, the recombinase is provided as a separate protein. The separate recombinase may form a dimer and bind to the donor template recombination site. The recombinase may be targeted to the loci of interest as a result of the insertion of the compatible recombination site that is also recognized by the recombinase. Thus, the recombinase may recognize the recombination site inserted at the DNA loci of interest and the recombination site on the donor and be targeted to the DNA loci of interest without any additional modifications to the recombinase.

In some embodiments, the prime-editing system further comprises a site-specific recombinase, and wherein the first and second extended regions are complementary to each other and introduce a serine integrase recombination site; and a donor template comprising a donor sequence for insertion into the target polypeptide and the complementary recombination site to the serine integrase recombination site.

### Codon Optimization

Embodiments of the various aspects described herein relate to polynucleotide molecules that encode one or more components of nuclease systems. In some cases, at least one or more regions of the polynucleotide molecule can be codon optimized for expression in a eukaryotic cell. In some embodiments, the polynucleotide molecules that encode one or more components of nuclease systems as described herein are optimized for expression in a mammalian cell or a plant cell. An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in International Patent Publication No. WO 2014/093622 (PCT/US2013/074667) as an example of a codon optimized sequence (from knowledge in the art and this disclosure, codon optimizing coding nucleic acid molecule(s), especially as to effector protein is within the ambit of the skilled artisan). Whilst this is preferred, it will be appreciated that other examples are possible and codon optimization for a host species other than human, or for codon optimization for specific organs is known. In some embodiments, a coding sequence encoding a protein effector polypeptide or guide nucleic acid described herein is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence.

Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (APTAGEN; Jacobus, P A), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid.

### Linkers

The various domains in the polypeptides and nucleic acids described herein can be linked via a direct bond (e.g., linked directly to each other) or a linker.

In some embodiments, the domains in a polypeptide are linked via a direct amide bond or a peptide linker. The term “peptide linker” as used herein denotes a peptide with amino acid sequences, which is in some embodiments of synthetic origin. It is noted that peptide linkers may affect folding of a given fusion protein, and may also react/bind with other proteins, and these properties can be screened for by known techniques. Exemplary peptide linkers include those that consist of glycine and serine residues, the so-called Gly-Ser polypeptide linkers. As used herein, the term “Gly-Ser polypeptide linker” refers to a peptide that consists of glycine and serine residues. In some embodiments, the peptide linker comprises the amino acid sequence (GxS)n (SEQ ID NO: 160) with G is glycine, S is serine, where x is 3, 4 or 5 and n is 1, 2, 3, 4, 5, 6, 7, 8, 9 or 10. In some embodiments, x is 3 and, n is 8, 9 or 10. In some embodiments, x is 4 and n is 6, 7, or 8. In some embodiments, x is 4 and n is 6 or 7. In some embodiments, x is 4 and n is 7. In some embodiments, the peptide linker comprises the amino acid sequence Ser (Gly₄Ser)ₙ (SEQ ID NO: 161) wherein n is an integer 1 to 10, respectively. In some embodiments, the peptide linker comprises the amino acid sequence (G4S) 6G2 (SEQ ID NO: 162). n some embodiments, the peptide linker comprises the amino acid sequence GSGGGSGGGGSGGGGS (SEQ ID NO: 163). In some embodiments, the peptide linker comprises the amino acid sequence GGSGG (SEQ ID NO: 164).

In some embodiments, the peptide linker comprises the amino acid sequence (X) n, where each X is independently aspartic acid (D) or glutamic acid (E), and n ranges from 1 to up to 10 or more.

Exemplary linkers, in addition to those described herein, include a string of histidine residues, e.g., His6 (SEQ ID NO: 165), sequences made up of Ala and Pro, varying the number of Ala-Pro pairs to modulate the flexibility of the linker; and sequences made up of charged amino acid residues e.g., mixing Glu and Lys. Flexibility can be controlled by the types and numbers of residues in the linker. See, e.g., Perham, 30 Biochem. 8501 (1991); Wriggers et al., 80 Biopolymers 736 (2005).

In some embodiments, the domains in a nucleic acid described herein are linked via a direct modified (e.g., phosphorothioate, imidp or MMI) or un-modified (e.g., phosphorothioate) internucleoside, or by a nucleotide linker. A “nucleotide linker” as used herein denotes a nucleotide sequence, which is in some embodiments of synthetic origin. In some embodiments, a nucleotide linker comprises from 1 to about 20 nucleotides. For example, the nucleotide linker comprises 1, 2, 3, 4, 5, 6, 7, 8, 9, 10, 11, 12, 13, 14, 15, 16, 17, 18, 19 or 20 nucleotides. In some embodiments, the nucleotide linker comprises 1, 2, 3, 4, 5, 6, 7, 8, 9, or 10 nucleotide. Some exemplary nucleotide linkers include, but are not limited to, GAAA and GAAAA.

Chemical linkers can comprise a direct bond or an atom such as oxygen or sulfur, a unit such as NH, C(O), C(O)NH, SO, SO₂, SO₂NH, or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂ heteroaryl, substituted or unsubstituted C₅-C₁₂ heterocyclyl, substituted or unsubstituted C₃-C₁₂ cycloalkyl, where one or more methylenes can be interrupted or terminated by O, S, S(O), SO₂, NH, or C(O). The linker can be 1 amino acid or more, 5 amino acids or more, 10 amino acids or more, 15 amino acids or more, 20 amino acids or more, 25 amino acids or more, 30 amino acids or more, 35 amino acids or more, 40 amino acids or more, 45 amino acids or more, 50 amino acids or more and beyond.

### Kits

A variety of kits and components can be prepared for use in the methods described herein, depending upon the intended use of the kit. Accordingly, in another aspect, provided herein is a kit comprising one or more components of the engineered, non-natural DNA and RNA targeting complexes described herein. A kit is any manufacture (e.g., a package or container) comprising one or more components of the engineered, non-natural DNA and RNA targeting complexes described herein. The manufacture can be promoted, distributed, or sold as a unit for performing the methods described herein.

The kits described herein can optionally comprise additional components and reagents. As will be appreciated by one of skill in the art, components of the kit can be provided in any desired form, e.g., in a lyophilized form, a liquid form, a solid form, or a concentrated. In some embodiments of the various aspects described herein, the kit can comprise ampoules, syringes, or the like.

In some embodiments, the kit can comprise informational material. The informational material can be descriptive, instructional, marketing or other material that relates to the methods described herein. The informational material of the kits is not limited in its form. In some embodiments, the informational material can include information about production of the reagents, concentration, date of expiration, batch or production site information, and so forth. In one embodiment, the informational material relates to methods for using or administering the components of the kit.

It is noted that the components of a kit can provided singularly or in any combination as a kit. Such a kit includes the components described herein and packaging materials thereof.

In some embodiments, the compositions in a kit can be provided in a watertight or gas tight container which in some embodiments is substantially free of other components of the kit. For example, the reagents described herein can be supplied in more than one container, e.g., it can be supplied in a container having sufficient reagent for a predetermined number of applications, e.g., 1, 2, 3 or greater. One or more components as described herein can be provided in any form, e.g., liquid, dried or lyophilized form. Liquids or components for suspension or solution of the reagents can be provided in sterile form and should not contain microorganisms or other contaminants. When the components described herein are provided in a liquid solution, the liquid solution preferably is an aqueous solution.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### Some Selected Definitions

For convenience, the meaning of some terms and phrases used in the specification, examples, and appended claims, are provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell can be a single cell as well as to a population of (i.e., more than one) cells. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g. Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etc. Sometimes a cell is not originating from a natural organism (e.g., a cell can be a synthetically made, sometimes termed an artificial cell). In some embodiments, cell is a human cell.

Cells suitable for use in the present invention include, but are not limited to, cells that are capable of differentiating completely or partially into a mature cell of the inner ear, e.g., a hair cell (e.g., an inner and/or outer hair cell), when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells.

The cell may be a prokaryotic cell or a eukaryotic cell. The cell may be a mammalian cell. The mammalian cell many be a non-human primate, bovine, porcine, rodent or mouse cell. The cell may be a non-mammalian eukaryotic cell such as poultry, fish or shrimp. The cell may be a therapeutic T cell or antibody-producing B-cell. The cell may also be a plant cell. The plant cell may be of a crop plant such as cassava, corn, sorghum, wheat, or rice. The plant cell may also be of an algae, tree or vegetable. The modification introduced to the cell by the present invention may be such that the cell and progeny of the cell are altered for improved production of biologic products such as an antibody, starch, alcohol or other desired cellular output. The modification introduced to the cell by the present invention may be such that the cell and progeny of the cell include an alteration that changes the biologic product produced.

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more nucleic acid modifications. If present, modifications to the nucleotide structure may be imparted before or after assembly of the polymer. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

Nucleic acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the nucleotide sequences described herein are also specifically contemplated and provided for herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are the same as a nucleotide sequence described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a nucleotide sequence described herein.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et ah, 1989, Molecular Cloning: A Laboratory Manual, 18.1-18.88. The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

Amino acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to an amino acid sequence described herein are also specifically contemplated and provided for herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a amino acid sequence described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

The terms “wild-type” or “wt” or “native” as used herein is meant an amino acid sequence or a nucleotide sequence that is found in nature, including allelic variations. A wild-type protein, polypeptide, antibody, immunoglobulin, IgG, polynucleotide, DNA, RNA, and the like has an amino acid sequence or a nucleotide sequence that has not been intentionally modified

As used herein, the term “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic necessary elements to promote transcriptional expression of an operably linked polynucleotide. Eukaryotic basal promoters typically, though not necessarily, contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner known to be attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” are used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation I of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with parameters of the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. a control level) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein, the term “Recombinase” refers to an enzyme that catalyzes recombination between two or more recombination sites (e.g., an acceptor and donor site). Recombinases useful in the present invention catalyze recombination at specific recombination sites which are specific polynucleotide sequences that are recognized by a particular recombinase. “Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the unidirectional recombinase, the continued presence of the recombinase cannot reverse the previous recombination event.

“Recombination sites” are specific polynucleotide sequences that are recognized by the recombinase enzymes described herein. Typically, two different sites are involved (in regards to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is again the core region.

Upon recombination between the attB and attP sites, and concomitant integration of a nucleic acid at the target, the recombination sites that flank the integrated DNA are referred to as “attL” and “aatR.” The attL and attR sites, using the terminology above, thus consist of BOP′ and POB′, respectively. In some representations herein, the “O” is omitted and attB and attP, for example, are designated as BB′ and PP′, respectively.

The terms “increased”, “increase”, “enhance”, or “activate” are all used herein to mean an increase by a statistically significant amount. In some embodiments, the terms “increased”, “increase”, “enhance”, or “activate” can mean an increase of at least 10% as compared to a reference level (e.g., a control level), for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a disease or disorder. A subject can be male or female.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Some embodiments of the technology described herein can be defined according to any of the following numbered paragraphs:

1. An IscB protein (protein effector polypeptide) comprising:
(a) an amino acid sequence that is at least 80% identical to the amino acid sequence of an IscB as provided by any one of SEQ ID NO: 2, wherein the amino acid sequence of the IscB protein comprises at least one, at least two, at least three, at least four, at least five, at least six, at least seven, at least eight, or at least nine mutations in an amino acid residue selected from the group consisting of amino acid residues 18, 34, 36, 37, 60, 66, 85, 97, 99, 100, 101, 104, 109, 112, 118, 136, 137, 155, 156, 157, 166, 168, 190, 204, 204, 205, 206, 208, 212, 214, 216, 218, 219, 229, 229, 230, 233, 233, 234, 234, 238, 239, 241, 242, 245, 246, 248, 252, 254, 255, 256, 257, 258, 279, 280, 291, 293, 298, 303, 304, 306, 307, 312, 319, 323, 328, 329, 331, 332, 338, 341, 353, 360, 368, 372, 376, 377, 378, 379, 380, 381, 382, 384, 385, 386, 388, 390, 391, 393, 405, 406, 408, 409, 410, 412, 413, 416, 417, 418, 419, 421, 421, 423, 425, 437, 439, 455, 462, 463, 478, 479, 533, 536, 564 and 565 of the amino acid sequence of SEQ ID NO: 2 (pEDIT071), and provided that, if the IscB comprises a single mutation, it is not Q60I, N66H, I85R, I85K, E137R, P405R, D413K, E409R or combination thereof; or
(b) an amino acid sequence that is at least 80% identical to the amino acid sequence of an IscB as provided by any one of SEQ ID NO: 8, wherein the amino acid sequence of the IscB protein comprises at least one, at least two, at least three, at least four, at least five, at least six, at least seven, at least eight, or at least nine mutations in an amino acid residue selected from the group consisting of amino acid residues 30, 46, 48, 49, 72, 78, 97, 109, 111, 112, 113, 116, 121, 124, 130, 148, 149, 167, 168, 169, 178, 180, 202, 216, 216, 217, 218, 220, 224, 226, 228, 230, 231, 241, 241, 242, 245, 245, 246, 246, 250, 251, 253, 254, 257, 258, 260, 264, 266, 267, 268, 269, 270, 291, 292, 303, 305, 310, 315, 316, 318, 319, 324, 331, 335, 340, 341, 343, 344, 350, 353, 365, 372, 380, 384, 388, 389, 390, 391, 392, 393, 394, 396, 397, 398, 400, 402, 403, 405, 417, 418, 420, 421, 422, 424, 425, 428, 429, 430, 431, 433, 433, 435, 437, 449, 451, 467, 474, 475, 490, 491, 545, 548, 576, and 577 of the amino acid sequence of SEQ ID NO: 8 (pEDIT071+NLS), and provided that, if the IscB comprises a single mutation, and optionally, if the IscB comprises a single mutation, the mutation is not Q72I, H78H, I97R, I97K, E149R, P417R, E421R, D425K, or combination thereof.

2. The IscB protein of paragraph 1, wherein the amino acid sequence of the IscB protein comprises:
(a) at least one, at least two, at least three, at least four, at least five, at least six, at least seven, at least eight, or at least nine mutations selected from the group consisting of variant C18R, A34R, D36R, T37R, Q60I, N66H, I85X, 185X, M99X, A101X, A104R, E109X, A112K, R118K, S136X, E137R, I155X, L156X, T157X, T166X, L168K, L190K, G204X, N205X, I206K, K208R, K212R, K214R, N216R, K218R, D219X, A229X, K230R, L233X, P234X, P238X, F239X, K241R, E242X, A245X, E246X, E248R, T252X, E254X, K255R, S256X, T257X, F258X, S279R, G280R, L291X, K293R, Q298K, E303X, M304K, K306R, R307K, C312X, T319K, E323X, E328R, Q329R, D331R, G332R, G338R, S341R, R353K, A360X, K368R, L372R, D376R, A377R, D378R, T379R, A380R, E381R, S382R, T384R, E385R, M386R, T388R, L390X, M391X, K393R, P405K, K406R, V408R, E409X, T410R, A412R, D413R, P416R, G417R, H418K, F419R, V421X, N423R, W425K, E437R, D439X, E455R, E462R, P463R, I533K, T536R, H478X, H479X, I564X, and T565X of the amino acid sequence provided in SEQ ID NO: 2, wherein X is any amino acid, and optionally, if the IscB comprises a single mutation, it is not Q60I, N66H, I85R, I85K, E137R, P405R, D413K, E409R or combination thereof; or
(b) at least one, at least two, at least three, at least four, at least five, at least six, at least seven, at least eight, or at least nine mutations selected independently from the group consisting of variant C30R, A46R, D48R, T49R, Q72I, N78H, I97X, I109X, M111X, A1113X, A116R, E121X, A124K, R130K, S148X, E149R, I167X, L168X, T169X, T178X, L180K, L202K, G216X, N217X, I218K, K2220R, K224R, K2226R, N228R, K230R, D231X, A241X, K242R, L245X, P246X, P250X, F251X, K253R, E254X, A257X, E258X, E260R, T264X, E266X, K267R, S268X, T269X, F270X, S291R, G292R, L303X, K2305R, Q310K, E315X, M316K, K318R, R319K, C324X, T331K, E335X, E340R, Q343R, G344R, G350R, S353R, R365K, A372X, K380R, L384R, D388R, A389R, D390R, T391R, A392R, E393R, S394R, T396R, E397R, M398R, T400R, L402X, M403X, K3405R, P417K, K418R, V420R, E421X, T422R, A424R, D425R, P428R, G429R, H430K, F431R, V433X, N435R, W437K, E449R, D451X, E467R, E474R, P475R, I545K, T548R, H490X, H491X, I576X, and T577X of the amino acid sequence SEQ ID NO: 8, wherein X is any amino acid, and optionally, if the IscB comprises a single mutation, the mutation is not Q72I, H78H, I97R, I97K, E149R, P417R, E421R, D425K, or combination thereof

3. The IscB protein of paragraph 1, wherein the amino acid sequence of the IscB protein comprises:
(a) at least one, at least two, at least three, at least four, at least five, at least six, at least seven, at least eight, or at least nine mutations selected from the group consisting of variant C18R, A34R, D36R, T37R, Q60I, N66H, I85X, 185X, M99X, A101X, A104R, E109X, A112K, R118K, S136X, E137R, I155X, L156X, T157X, T166X, L168K, L190K, G204X, N205X, I206K, K208R, K212R, K214R, N216R, K218R, D219X, A229X, K230R, L233X, P234X, P238X, F239X, K241R, E242X, A245X, E246X, E248R, T252X, E254X, K255R, S256X, T257X, F258X, S279R, G280R, L291X, K293R, Q298K, E303X, M304K, K306R, R307K, C312X, T319K, E323X, E328R, Q329R, D331R, G332R, G338R, S341R, R353K, A360X, K368R, L372R, D376R, A377R, D378R, T379R, A380R, E381R, S382R, T384R, E385R, M386R, T388R, L390X, M391X, K393R, P405K, K406R, V408R, E409X, T410R, A412R, D413R, P416R, G417R, H418K, F419R, V421X, N423R, W425K, E437R, D439X, E455R, E462R, P463R, I533K, T536R, H478N, H479N, I564K, I564H, I564N, I564Q, I564C, I564M, I564T, I564D, T565R, T565K, T565H, T565C, T565D, T565G, H478C, H478P, H479C, and H479M of the amino acid sequence provided in SEQ ID NO: 2, and optionally, if the IscB comprises a single mutation, it is not Q60I, N66H, I85R, I85K, E137R, P405R, D413K, E409R or combination thereof; or
(b) at least one, at least two, at least three, at least four, at least five, at least six, at least seven, at least eight, or at least nine mutations selected independently from the group consisting of C30R, A46R, D46R, T49R, Q72I, N78H, I97R, I97K, D109R, D109K, M111R, M111K, A112K, A113R, A113K, A116R, E121R, E121K, A124K, R130K, S148R, S148K, E149R, E149K, 1167R, 1167K, L168R, L168K, T169R, T169K, T178R, T178K, L180K, L1202K, G216R, G216K, N217R, N217K, I218K, K220R, K224R, K2226R, N228R, K230R, D231R, D231K, A241R, A241K, K242R, L245R, L245K, P246R, P246K, P250R, P250K, F251R, F251K, K253R, E254R, E2454K, A257R, A257K, E258R, E2458K, E260R, T264R, T264K, E266R, E266K, K267R, S268R, S268K, T2569R, T2569K, F270R, F270K, S291R, G292R, L303R, L303K, K305R, Q310K, E315R, E315K, M316K, K318R, R319K, C324R, C324K, T331K, E335R, E335K, E340R, Q341R, D343R, G344R, G350R, S353R, R365K, A372R, A372K, K380R, L384R, D388R, A389R, D390R, T391R, A392R, E393R, S394R, T396R, E397R, M398R, T400R, L402R, L402K, M4031R, M403K, K405R, P417K, K419R, V420R, E421K, E421R, T422R, A424R, D425R, D425K, P428R, G429R, H430K, F431R, V433R, V433K, N435R, W437K, E449R, D451R, D451K, E4567R, E474R, P475R, I545K, T548R, H490N, H491N, I576K, I576H, I576N, I576Q, I576C, I576M, I576T, I5676D, T577R, T577K, T577H, T577C, T577D, T577G, H490C, H490P, H491C, and H491M of the amino acid sequence provided in SEQ ID NO: 8. Optionally, if the IscB comprises a single mutation, the mutation is not Q72I, H78H, 197R, I97K, E149R, P417R, E421R, D425K, or combination thereof.

4. The IscB protein of any one of paragraphs 1-3, wherein the IscB protein comprises: (i) at least E409R, D413K, 533K, and at least one other mutation listed in Table A; or (ii) at least E421R, D425K, 545K, and at least one other mutation listed in Table A2.

5. The IscB protein of any one of paragraphs 1-3, wherein the IscB protein comprises: (i) at least E409R, D413K, 533K E137K, N66H, A100K, T536R, and at least one other mutation listed in Table A; or (ii) at least E421R, D425K, 545K E149K, N78H, A112K, T548R, and at least one other mutation listed in T3able A2.

6. The IscB protein of any one of paragraphs paragraph 1-5 comprising: an amino acid sequence that is at least 85%, at least 90%, at least 92%, at least 95%, at least 96%, at least 97%, at least 98%, at least 99%, or at least 99.5% identical to the amino acid sequence of an IscB as provided by SEQ ID NO: 2 or 8.

7. The IscB protein of any one of paragraphs 1-6, wherein the IscB exhibits an increased activity on a target sequence that does not comprise the canonical TAM (5′-ATAAA-3′) at its 3′ end as compared to IscB as provided by SEQ ID NO: 2 or 8.

8. The IscB protein of any one of paragraphs 1-7, wherein the IscB protein exhibits an activity on a target sequence having a 3′ end that is not directly adjacent to the canonical TAM sequence (5′-ATAAA-3′) that is at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of IscB as provided by SEQ ID NO: 2 or 8 on the same target sequence.

9. The IscB protein of any one of paragraphs 6-8, wherein the activity is measured by a nuclease assay, a deamination assay, or a transcriptional activation assay.

10. The IscB protein of any one of paragraphs 1-3, wherein the IscB protein comprises: (i) a E409R, D413K, and 533K mutations of the amino acid sequence provided in SEQ ID NO: 2; or (ii) a E421R, D425K, and 545K mutations of the amino acid sequence provided in SEQ ID NO: 8.

11. The IscB protein of any one of paragraphs 1-3 or 10, wherein the IscB protein comprises: (i) a E137K, N66H, A100K, and/or T536R mutation of the amino acid sequence provided in SEQ ID NO: 2; or (ii) a E149K, N78H, A112K, and/or T548R mutation of the amino acid sequence provided in SEQ ID NO: 8.

12. The IscB protein of paragraph 11, wherein the IscB protein comprises: (i) E409R, D413K, 533K E137K, N66H, A100K, and T536R mutations of the amino acid sequence provided in SEQ ID NO: 2; or (ii) E421R, D425K, 545K E149K, N78H, A112K, and T548R mutations of the amino acid sequence provided in SEQ ID NO: 8.

13. The IscB protein of any one of paragraphs 1-11, wherein the IscB exhibits an increased activity on a target sequence comprising a TAM sequence selected from the group consisting of RTAAA, ATRAA, ATRMM, ATRA, ATRRA, ATARA, ATRRAC, ATAAAC, ATARM, ATRAM, ATRRM, ATRMA, ATANA, ATRR, ATR, ATGAC, ATAA, ATAR, ATGMC, ATGAA, and ATRAR at its 3′ end as compared to IscB as provided by SEQ ID NO: 2 or 8, wherein R is a purine and M is A or C.

14. The IscB protein of paragraph 13, wherein the IscB comprises: (i) at least one, at least two, at least 3 or at least 4 mutations selected from the group consisting of variant H478X, H479N, I564X, T565X, H479C, and H479M of the amino acid sequence provided in SEQ ID NO: 2, wherein X is any amino acid; or (ii) at least one, at least two, at least 3 or at least 4 mutations selected from the group consisting of variant H490X, H491N, I576X, T577X, H491C, and H491M of the amino acid sequence provided in SEQ ID NO: 8, wherein X is any amino acid

15. The IscB protein of paragraph 14, wherein the IscB comprises at least one, at least two, at least 3 or at least 4 mutations selected from the group consisting of variant H478N, H479N, I564K, I564H, I564N, I564Q, I564C, 1564M, I564T, 1564D, T565R, T565K, T565H, T565C, T565D, T565G, H478C, H478P, H479C, and H479M of the amino acid sequence provided in SEQ ID NO: 2; or (ii) H490N, H491N, I576K, I576H, I576N, I576Q, I576C, I576M, I576T, I576D, T577R, T577K, T577H, T577C, T577D, T577G, H490C, H490P, H491C, and H491M of the amino acid sequence provided in SEQ ID NO: 8.

16. The IscB protein of any one of paragraphs 1-15, wherein the IscB exhibits an increased activity on a target sequence comprising a TAM sequence is ATR.

17. The IscB protein of paragraph 13, comprising at least 3, at least 4, at least 5, at least 6, at least 7, at least 8, at least 9, at least 10 or at least 11 amino sequences swap at position 560-571 of SEQ ID NO: 2, or at least 3, at least 4, at least 5, at least 6, at least 7, at least 8, at least 9, at least 10 or at least 11 amino sequences swap at position 572-583 of SEQ ID NO: 8.

18. The IscB protein of paragraphs 17, comprising at least 3, at least 4, at least 5, or at least 6 amino sequences swap at position Q563-K568 of SEQ ID NO: 2 or at least 3, at least 4, at least 5, or at least 6 amino sequences swap at position Q575-K580 of SEQ ID NO: 8.

19. The IscB protein of paragraph 18, comprising six amino sequences swap at position Q563-K568 of SEQ ID NO: 2 or at position is Q575-K580 of SEQ ID NO: 8 is selected from QTSGGR (SEQ ID NO: 11), RSSGGK (SEQ ID NO: 12), QQSKGR (SEQ ID NO: 13), QQGRGR (SEQ ID NO: 14), QQNKGR (SEQ ID NO: 15), and QKDKGA (SEQ ID NO: 16).

20. The IscB protein of paragraph 19, wherein the IscB exhibits an increased activity on a target sequence comprising a TAM sequence is ATR.

21. The IscB protein of paragraph 17, comprising at least 7, at least 8, at least 9, at least 10 or at least 11 amino sequences swap at position 560-571 of SEQ ID NO: 2, or at position 572-583 of SEQ ID NO: 8.

22. The IscB protein of paragraph 18, comprising the amino acid sequence MSGQHCHGAYLR (SEQ ID NO: 51), LSGNSHYGQYYM (SEQ ID NO: 52), MTGQHCHGNYFR (SEQ ID NO: 53), MTGQQNKGDRYF (SEQ ID NO: 54), KSGQLTNGQYFR (SEQ ID NO: 55), LKGNACNGAYFH (SEQ ID NO: 56), SSGTHAKSYFR (SEQ ID NO: 57), LGGQKNNGVYFY (SEQ ID NO: 58), VTGSSGAGKYVR (SEQ ID NO: 59), VRSVHSYGKYVR (SEQ ID NO: 60), VQGIKNRGRYLH (SEQ ID NO: 61), LRGTSSYGKTVL (SEQ ID NO: 62), VKGVFNYGKWVR (SEQ ID NO: 63), VKGKHNDRWI (SEQ ID NO: 64), VKGVQNRGKYIK (SEQ ID NO: 65), IKGSHCNGSRLI (SEQ ID NO: 66), SGGVMSYGRYIT (SEQ ID NO: 67), SKGNKNKGKSIL (SEQ ID NO: 68), AIGTHCKGKRVI (SEQ ID NO: 69), VKGVFNRGTWVR (SEQ ID NO: 70), CKGMHSYGRYVL (SEQ ID NO: 71), IGGTQNKGRYAI (SEQ ID NO: 72), or TKGTHCKGTRIM (SEQ ID NO: 73) at position M560-R571 of SEQ ID NO: 2.

23. The IscB protein of paragraph 20, wherein the IscB exhibits an increased activity on a target sequence comprising a TAM sequence is ATRR.

24. The IscB protein of any one of paragraphs 1-13, wherein the IscB exhibits an increased activity on a target sequence comprising a TAM sequence is RTAAA.

25. The IscB protein of paragraph 24, wherein the IscB comprises a mutation at position 478 and/or 479 of the amino acid sequence provided in SEQ ID NO: 2, or at position 490 and/or 491 of the amino acid sequence provided in SEQ ID NO: 2

26. The IscB protein of paragraph 25, wherein the IscB comprises: (i) a mutation H478N, H479N, H478C and/or H478P of the amino acid sequence provided in SEQ ID NO: 2; or (ii) a mutation H490N, H491N, H490C and/or H490P of the amino acid sequence provided in SEQ ID NO: 8.

27. The IscB protein of any one of paragraphs 1-26, wherein the IscB protein exhibits lower off-target activity as compared to an off-target activity of the IscB domain as provided by SEQ ID NO: 2 or 8.

28. The IscB protein of any one of paragraphs 1-27, wherein the IscB protein further comprises a nuclear localization signal (NLS) sequence.

29. The IscB protein of paragraph 28, wherein the NLS comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with any one of the NLS sequences show in in Table C, or a homolog or ortholog thereof.

30. The IscB protein of paragraph 28 or 29, wherein the NLS is linked to N-terminal of the IscB protein.

31. The IscB protein of paragraph 28 or 29, wherein the NLS is linked to C-terminal of the IscB protein.

32. A fusion protein comprising (i) the IscB protein of any one of paragraphs 1-31 and (ii) an effector domain.

33. The fusion protein of paragraph 32, wherein the effector domain is a domain that comprises nuclease activity, nickase activity, recombinase activity, deaminase activity, methyltransferase activity, methylase activity, acetylase activity, acetyltransferase activity, transcriptional activation activity, or transcriptional repression activity.

34. The fusion protein of paragraph 32 or 33, wherein the effector domain is a nucleic acid editing domain.

35. The fusion protein of paragraph 32 or 33, wherein effector domain comprises a deaminase domain, a reverse transcriptase, or a functional fragment thereof.

36. The fusion protein of paragraph 35 wherein the deaminase domain is a cytidine deaminase domain.

37. The fusion protein of paragraph 36, wherein the cytidine deaminase domain is an apolipoprotein B mRNA-editing complex (APOBEC) family deaminase.

38. The fusion protein of paragraph 36 or 37, wherein the cytidine deaminase domain is at least 80%, at least 85%, at least 90%, at least 92%, at least 95%, at least 96%, at least 97%, at least 98%, at least 99%, at least 99.5%, or 100% identical to the cytidine deaminase domain of any one of the sequences in Table C.

39. The fusion protein of paragraph 35, wherein the deaminase domain is an adenosine deaminase domain.

40. The fusion protein of paragraph 35, further comprising a second adenosine deaminase.

41. The fusion protein of paragraph 36, wherein the first adenosine deaminase domain and the second adenosine deaminase domain comprises an ecTadA domain, or variant thereof.

42. The fusion protein of paragraph 36 or 37, wherein the first adenosine deaminase domain and the second adenosine deaminase domain comprise independently an amino acid sequence that is at least 80%, at least 85%, at least 90%, at least 92%, at least 95%, at least 96%, at least 97%, at least 98%, at least 99%, at least 99.5%, or 100% identical to the cytidine deaminase domain of any one of the sequences in Table D1.

43. The fusion protein of any one of paragraphs 32-42, wherein the fusion protein further comprises a uracil glycosylase inhibitor (UGI) domain.

44. The fusion protein of paragraph 43, wherein the UGI domain comprises an amino acid sequence having is at least 80%, at least 85%, at least 90%, at least 92%, at least 95%, at least 96%, at least 97%, at least 98%, at least 99%, at least 99.5%, or 100% identical to any one of the sequences in Table E.

45. The fusion protein of any one of paragraphs 43-44, wherein the fusion protein further comprises a second UGI domain.

46. A ωRNA comprising a conserved portion of a ωRNA (parent ωRNA) comprising one or more stem-loop structures, wherein at least one of the stem-loop structures comprises a modification, and wherein a conserved portion of the ωRNA is shorter than the conserved portion of the parent ωRNA and is capable of binding with a transposon family encoded nuclease or a homolog or ortholog thereof.

47. The ωRNA of paragraph 46, wherein the conserved region of the ωRNA comprises at least two stem-loop structures and wherein said modification is in a stem-loop structure near to the 5′-end (5′-stem-loop) of the conserved region ωRNA.

48. The ωRNA of paragraph 47, wherein said modification comprises deletion of from about 3 to about 46 nucleotides from the 5′-stem-loop.

49. The ωRNA of paragraph 47 or 48, wherein said modification comprises deletion of 3, 7, 11, 15, 18, 22, 26, 30, 34, 37 or 41 nucleotides from the 5′-stem-loop.

50. The ωRNA of paragraph 4, wherein said modification comprises deletion of 15, 18, 30, 37 or 41 nucleotides from the 5′-stem-loop.

51. The ωRNA of paragraph 47 or 48, wherein said modification comprises deletion of 8, 12, 16, 20, 23, 27, 31, 35, 39, 42 or 46 nucleotides from the 5′-stem-loop.

52. The ωRNA of paragraph 51, wherein said modification comprises deletion of 20, 23, 35, 42 or 46 nucleotides from the 5′-stem-loop.

53. The ωRNA of any one of paragraphs 47-52, wherein nucleotides at position 12-57, 14-55, 16-54, 18-52, 20-50, 22-48, 24-46, 26-45, 28-43, 30-41 or 32-39 are deleted (counting from 5′-end, e.g., 5′-end of the conserved region of the parent ωRNA).

54. The ωRNA of paragraph 53, wherein nucleotides at position 12-57, 14-55, 18-52, 24-46, or 26-45 are deleted (counting from 5′-end, e.g., 5′-end of the conserved region of the parent ωRNA).

55. The ωRNA of any one of paragraphs 47-54, wherein the deleted nucleotides are replaced with a linker (e.g., a loop portion or pin-loop).

56. The ωRNA of paragraph 55, wherein the linker is single-stranded nucleic acid comprising from about from about 4 nucleotides to about 15 nucleotides.

57. The ωRNA of any one of paragraphs 47-56, wherein a loop portion (pin-loop) of the 5′-stem-loop comprises from 4, 5 or 6 nucleotides.

58. The ωRNA of any one of paragraphs 47-57, wherein a loop portion of the 5′-stem-loop comprises the nucleotide sequence GAAA or GAAAA.

59. The ωRNA of any one of paragraphs 47-58, wherein at least a portion of the 5′-stem-loop is within 45 nucleotides, counting from 5′-end, e.g., 5′-end of the conserved region of the parent ωRNA.

60. The ωRNA of any one of paragraphs 47-59, wherein at least a portion of the stem of the 5′-stem-loop is within 25 nucleotides, counting from 5′-end, e.g., 5′-end of the conserved region of the parent ωRNA.

61. The ωRNA of any one of paragraphs 47-60, wherein a stem portion of the 5′-stem-loop comprises a double-stranded region comprising from about 8 nucleotide base-pairs to about 25 nucleotide base-pairs.

62. The ωRNA of any one of paragraphs 47-61, wherein a stem portion of the 5′-stem-loop comprises a double-stranded region comprising about 9 nucleotide base-pairs, about 10 nucleotide base-pairs, about 11 nucleotide base-pairs, about 12 nucleotide base-pairs, about 13 nucleotide base-pairs, about 14 nucleotide base-pairs, about 15 base pairs nucleotide base-pairs, about 16 base pairs nucleotide base-pairs, about 17 base pairs nucleotide base-pairs, about 18 base pairs nucleotide base-pairs, about 19 base pairs nucleotide base-pairs, or about 20 base pairs nucleotide base-pairs.

63. The ωRNA of any one of paragraphs 47-62, wherein a stem portion of the 5′-stem-loop comprises a double-stranded region comprising about 9 nucleotide base-pairs, about 10 nucleotide base-pairs, about 11 nucleotide base-pairs, or about 12 nucleotide base-pairs.

64. The ωRNA of any one of paragraphs 47-63, wherein a stem portion of the 5′-stem-loop comprises a double-stranded region comprises 10 nucleotide base-pairs.

65. The ωRNA of any one of paragraphs 47-64, wherein the 5′-stem-loop comprises from about 20 nucleotides to about 65 nucleotides.

66. The ωRNA of any one of paragraphs 47-65, wherein the 5′-stem-loop comprises about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, or about 45 nucleotides.

67. The ωRNA of any one of paragraphs 47-66, wherein the 5′-stem-loop comprises 23, 24, 25, 26 or 27 nucleotides.

68. The ωRNA of any one of paragraphs 47-67, wherein the 5′-stem-loop comprises a nucleic acid modification, optionally selected from the group consisting of nucleobase modifications, sugar modifications, inter-sugar linkage modifications (modified internucleoside linkages), conjugates (e.g., ligands), and any combinations thereof.

69. The ωRNA of paragraph 68, wherein a stem (double-stranded) region of the 5′-stem-loop comprises a nucleic acid modification.

70. The ωRNA of paragraph 68 or 69, wherein a loop region (pin-loop) of the 5′-stem-loop comprises a nucleic acid modification.

71. The ωRNA of any one of paragraphs 68-70, wherein said nucleic acid modification is a modified nucleotide selected from the group consisting of 2′-O-methyl(2′-OMe) nucleotides, 2′-fluoro (2′-F) nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl(2′-MOE) nucleotides, 2′-O-allyl nucleotides, 2′-C-allyl ribose nucleotides, 2′-O-N-methylacetamido (2′-O-NMA) nucleotides, a 2′-O-dimethylaminoethoxyethyl(2′-O-DMAEOE) nucleotides, 2′-O-aminopropyl(2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a non-natural or modified nucleobase; or a combination thereof.

72. The ωRNA of any one of paragraphs 68-71, wherein a stem (double-stranded) region of the 5′-stem-loop comprises a 2′-O-methyl modified nucleotide.

73. The ωRNA of any one of paragraphs 68-71, wherein a stem (double-stranded) region of the 5′-stem-loop comprises a 2′-fluoro modified nucleotide.

74. The ωRNA of any one of paragraphs 68-71, wherein a nucleotide in a stem (double-stranded) region of the 5′-stem-loop comprises a non-natural or modified nucleobase.

75. The ωRNA of any one of paragraphs 68-74, wherein a stem (double-stranded) region of the 5′-stem-loop comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

76. The ωRNA of paragraph 68-75, wherein a loop (single-stranded) region (pin-loop) of the 5′-stem-loop comprises a 2′-O-methyl modified nucleotide.

77. The ωRNA of any one of paragraphs 68-75, wherein a loop (single-stranded) region (pin-loop) of the 5′-stem-loop comprises a 2′-fluoro modified nucleotide.

78. The ωRNA of any one of paragraphs 68-77, wherein a nucleotide in a loop (single-stranded) (pin-loop) region of the 5′-stem-loop comprises a non-natural or modified nucleobase.

79. The ωRNA of any one of paragraphs 68-78, wherein a loop (single-stranded) (pin-loop) region of the 5′-stem-loop comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

80. The ωRNA of any one of paragraphs 47-79, wherein a stem (double-stranded) region of the 5′-stem-loop comprises a duplex stabilizing modification, optionally the duplex stabilizing modification is 2′-F nucleotide, 2′-OMe nucleotide, 2′-methoxyethyl nucleotide, 2,6-diaminopurine nucleotide, 5-methyl cytidine, N4-ethyl cytidine, 5-propynyl cytidine, 5-propynyl uridine, 5-hydroxybutynl-2′-deoxyuridine, 8-aza-7-deazaguanosine, a locked nucleic acid (LNA), and/or covalent cross-linking of two strands of the duplex.

81. The ωRNA of any one of paragraphs 47-80, wherein two nucleotides in the stem of the 5′-loop-stem are covalently linked to each other via a position other than 5′ and 3′.

82. The ωRNA of any one of paragraphs 46-81, wherein the conserved region of the ωRNA comprises at least two stem-loop structures and wherein said modification is in a stem-loop structure proximal to the 3′-end (3′-stem-loop) of the conserved region of the ωRNA.

83. The ωRNA of paragraph 82, wherein said modification comprises deletion of from about 1 to about 22 nucleotides from the 3′-stem-loop.

84. The ωRNA of paragraph 82 or 83, wherein said modification comprises deletion of 2, 6, 8, 10, 12, 16, or 20 nucleotides from the 3′-stem-loop.

85. The ωRNA of paragraph 84, wherein said modification comprises deletion of 2, 10, 12, or 16 nucleotides from the 3′-stem-loop.

86. The ωRNA of paragraph 82-85, wherein said modification comprises deletion of 2, 10, 12 or 16 nucleotides from the 5′-stem-loop.

87. The ωRNA of paragraph 86, wherein said modification comprises deletion of 2, 10, 12, or 16 nucleotides from the 3′-stem-loop.

88. The ωRNA of any one of paragraphs 82-87, wherein nucleotides at position 159-172, 160-171, 161-170, 163-168, 158-173, 156-175 or 154-177 are deleted (counting from 5′-end, e.g., of the parent ωRNA).

89. The ωRNA of paragraph 88, wherein nucleotides at position 159-172, 163-168, 158-173, or 156-175 are deleted (counting from 5′-end, e.g., of the parent ωRNA).

90. The ωRNA of any one of paragraphs 82-89, wherein the deleted nucleotides in the 3′-stem-loop are replaced with a linker (e.g., a loop portion or pin-loop).

91. The ωRNA of paragraph 90, wherein the linker is a single-stranded nucleic acid comprising from about from about 4 nucleotides to about 15 nucleotides.

92. The ωRNA of any one of paragraphs 82-91, wherein a loop portion of the 5′-stem-loop comprises from 4, 5 or 6 nucleotides.

93. The ωRNA of any one of paragraphs 82-92, wherein a loop portion of the 5′-stem-loop comprises the nucleotide sequence GAAA or GAAAA.

94. The ωRNA of any one of paragraphs 82-93, wherein at least a portion of the 3′-stem-loop is within 50 nucleotides, counting from 3′-end of the modified ωRNA.

95. The ωRNA of any one of paragraphs 82-94, wherein at least a portion of the stem of the 5′-stem-loop is within 45 nucleotides, counting from 3′-end of the modified ωRNA.

96. The ωRNA of any one of paragraphs 82-95, wherein a stem portion of the 3′-stem-loop comprises a double-stranded region comprising from about 3 nucleotide base-pairs to about 10 nucleotide base-pairs.

97. The ωRNA of any one of paragraphs 82-96, wherein a stem portion of the 3′-stem-loop comprises a double-stranded region comprising about 3 nucleotide base-pairs, about 4 nucleotide base-pairs, about 5 nucleotide base-pairs, about 6 nucleotide base-pairs, about 7 nucleotide base-pairs, or about 8 nucleotide base-pairs.

98. The ωRNA of any one of paragraphs 82-97, wherein a stem portion of the 3′-stem-loop comprises a double-stranded region comprising about 3 nucleotide base-pairs, about 4 nucleotide base-pairs, about 5 nucleotide base-pairs, or about 6 nucleotide base-pairs.

99. The ωRNA of any one of paragraphs 82-98, wherein a stem portion of the 5′-stem-loop comprises a double-stranded region comprises 3 or 4 nucleotide base-pairs.

100. The ωRNA of any one of paragraphs 82-99, wherein the 3′-stem-loop comprises from about 8 nucleotides to about 25 nucleotides.

101. The ωRNA of any one of paragraphs 78-100, wherein the 3′-stem-loop comprises about 9, about 10, about 11, about 12, about 13, about 14, about 15, about 16, about 17, about 18, about 19, or about 20 nucleotides.

102. The ωRNA of any one of paragraphs 82-101, wherein the 3′-stem-loop comprises 9, 10, 11, 12, 13, 14 or 15 nucleotides.

103. The RNA of any one of paragraphs 78-102, wherein the 3′-stem-loop comprises a nucleic acid modification, optionally selected from the group consisting of nucleobase modifications, sugar modifications, inter-sugar linkage modifications (modified internucleoside linkages), conjugates (e.g., ligands), and any combinations thereof.

104. The ωRNA of paragraph 103, wherein a stem (double-stranded) region of the 3′-stem-loop comprises a nucleic acid modification.

105. The ωRNA of paragraph 103 or 104, wherein a loop (single-stranded) region of the 3′-stem-loop comprises a nucleic acid modification.

106. The ωRNA of any one of paragraphs 103-105, wherein said nucleic acid modification is a modified nucleotide selected from the group consisting of 2′-O-methyl(2′-OMe) nucleotides, 2′-fluoro (2′-F) nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl(2′-MOE) nucleotides, 2′-O-allyl nucleotides, 2′-C-allyl ribose nucleotides, 2′-O-N-methylacetamido (2′-O-NMA) nucleotides, a 2′-O-dimethylaminoethoxyethyl(2′-O-DMAEOE) nucleotides, 2′-O-aminopropyl(2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a non-natural or modified nucleobase; or a combination thereof.

107. The ωRNA of any one of paragraphs 82-106, wherein a stem (double-stranded) region of the 3′-stem-loop comprises a 2′-O-methyl modified nucleotide.

108. The ωRNA of any one of paragraphs 82-107, wherein a stem (double-stranded) region of the 3′-stem-loop comprises a 2′-fluoro modified nucleotide.

109. The ωRNA of any one of paragraphs 82-108, wherein a nucleotide in a stem (double-stranded) region of the 5′-stem-loop comprises a non-natural or modified nucleobase.

110 The ωRNA of any one of paragraphs 82-109, wherein a stem (double-stranded) region of the 3′-stem-loop comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

111. The ωRNA of any one of paragraphs 82-110, wherein a loop (single-stranded) region of the 5′-stem-loop comprises a 2′-O-methyl modified nucleotide.

112. The ωRNA of any one of paragraphs 82-111, wherein a loop (single-stranded) region of the 5′-stem-loop comprises a 2′-fluoro modified nucleotide.

113. The ωRNA of any one of paragraphs 82-112, wherein a nucleotide in a loop (single-stranded) region of the 3′-stem-loop comprises a non-natural or modified nucleobase.

114. The ωRNA of any one of paragraphs 82-113, wherein a loop (single-stranded) region of the 5′-stem-loop comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

115. The ωRNA of any one of paragraphs 82-114, wherein two nucleotides in the stem of the 5′-loop-stem are covalently linked to each other via a position other than 5′ and 3′.

116. The ωRNA of any one of paragraphs 82-115, wherein two nucleotides in the stem of the 5′-loop-stem are covalently linked to each other via a position other than 5′ and 3′.

117. The ωRNA of any one of paragraphs 46-116, wherein the conserved region of the ωRNA comprises at least three stem-loop structures.

118. The ωRNA of any one of paragraphs 46-117, wherein at least one (e.g., 1, 2, 3, 4, 5, 6, 7 or 8) nucleotide in a loop region of a stem-loop forms a base-pair with a nucleotide outside said stem-loop.

119. The ωRNA of paragraph 118, wherein at least one (e.g., one or both) member of the base-pair comprises a modification capable of enhancing base-pairing.

120. The ωRNA of paragraph 119, wherein the base-pair nucleotide in the loop comprises the modification capable of enhancing base-pairing.

121. The ωRNA of paragraph 119 or 120, wherein the base-pair nucleotide outside said stem-loop comprises the modification capable of enhancing base-pairing.

122. The ωRNA of any one of paragraphs 119-121, wherein said modification capable of enhancing base-pairing is 2′-F modified nucleotide, 2′-OMe modified nucleotide, 2′-methoxyethyl modified nucleotide, 2,6-diaminopurine nucleotide, 5-methyl cytidine, N4-ethyl cytidine, 5-propynyl cytidine, 5-propynyl uridine, 5-hydroxybutynl-2′-deoxyuridine, 8-aza-7-deazaguanosine, or a locked nucleic acid (LNA).

123. The ωRNA of any one of paragraphs 119-122, wherein said modification capable of enhancing base-pairing is in at an internal (i.e., not a 5′-stem-loop or 3′-stem-loop) stem-loop (internal-hairpin) of the ωRNA.

124. The ωRNA of any one of paragraphs 119-123, wherein said modification capable of enhancing base-pairing is in a loop (single-stranded) region (pin-loop) of an internal (i.e., not a 5′-stem-loop or 3′-stem-loop) stem-loop (internal-hairpin) of the ωRNA.

125. The ωRNA of any one of paragraphs 46-124, wherein the ωRNA (e.g., a conserved region) is from about 50 to about 90 nucleotides shorter than the parent ωRNA.

126. The ωRNA of any one of paragraphs 46-125, wherein the ωRNA (e.g., a conserved region) is from about 55 to about 85 nucleotides shorter than the parent ωRNA.

127. The ωRNA of any one of paragraphs 46-126, wherein the ωRNA (e.g., a conserved region) is about 60, about 61, about 62, about 63, about 64, about 65, about 66, about 67, about 68, about 69, about 70, about 71, about 72, about 73, about 74, about 75, about 76, about 77, about 78, about 79, or about 80 nucleotides shorter than the parent ωRNA.

128. The ωRNA of any one of paragraphs 46-127, wherein the ωRNA (e.g., a conserved region) is from about 130 nucleotides to about 190 nucleotides in length.

129 The ωRNA of any one of paragraphs 46-128, wherein the ωRNA (e.g., a conserved region) is from about 135 nucleotides to about 180 nucleotides in length.

130 The ωRNA of any one of paragraphs 46-129, wherein the ωRNA (e.g., a conserved region) is from about 140 nucleotides to about 170 nucleotides in length.

131. The ωRNA of any one of paragraphs 46-130, wherein the ωRNA (e.g., a conserved region) is from about 140, about 141, about 142, about 143, about 144, about 145, about 146, about 147, about 148, about 149, about 150, about 151, about 152, about 153, about 1454, about 155, about 156, about 157, about 158, about 159, or about 160 nucleotides in length.

132. The ωRNA of any one of paragraphs 46-131, wherein the ωRNA comprises a 5′ end modification.

133. The ωRNA of any one of paragraphs 46-132, wherein the ωRNA comprises a 3′ end modification.

134. The ωRNA of any one of paragraphs 46-133, wherein the ωRNA comprises a 5′ end modification and a 3′ end modification.

135. The ωRNA of any one of paragraphs 132-134, wherein the 5′ and/or 3′ end modification comprises a protective end modification.

136. The ωRNA of paragraph 135, wherein the protective end modification is a modified nucleotide selected from 2′-O-methyl(2′-OMe) modified nucleotide, 2′-O-(2-methoxyethyl)(2′-O-moe) modified nucleotide, a 2′-fluoro (2′-F) modified nucleotide, a phosphorothioate (PS) linkage between nucleotides, a 3′->3′ linkage between nucleotides, an inverted abasic modified nucleotide, or combinations thereof.

137. The ωRNA of any one of paragraphs 132-136, wherein the 3′ end modification comprises any one of:
i. a modification of any one or more of the last 7, 6, 5, 4, 3, 2, or 1 nucleotides;
ii. one modified nucleotide;
iii. two modified nucleotides;
iv. three modified nucleotides;
v. four modified nucleotides;
vi. five modified nucleotides;
vii. six modified nucleotides; or
viii. seven modified nucleotides.

138. The ωRNA of any one of paragraphs 132-137, wherein the 3′ end modification
i. a modified internucleoside linkage (e.g., PS, imidp or MMI linkage) between nucleotides;
ii. a 2′-OMe nucleotide;
iii. a 2′-O-MOE nucleotide;
iv. a 2′-F nucleotide;
v. a LNA nucleotide
vi. a 3′->3′ linkage between nucleotides;
vii. an inverted abasic nucleotide; and
viii. a combination of one or more of (i.)-(vii.).

139. The ωRNA of any one of paragraphs 132-138, wherein the 3′ end modification comprises one or more of:
i. a modified internucleoside linkage (e.g., PS, imidp or MMI linkage) between the last and second to last nucleotides;
ii a modified internucleoside linkage (e.g., PS, imidp or MMI linkage) between the last and second to last nucleotide and a PS linkage between the second and third to last nucleotides;
iii. modified internucleoside linkages (e.g., PS and/or MMI linkage) between any one or more of the last four nucleotides;
iv. modified internucleoside linkages (e.g., PS and/or MMI linkage) between any one or more of the last five nucleotides; or
v. modified internucleoside linkages (e.g., PS and/or MMI linkage) between any one or more of the last 2, 3, 4, 5, 6, 7, 8, 9, or 10 nucleotides.

140. The ωRNA of any one of paragraphs 132-139, wherein the 3′ end modification comprises at least one 2′-F nucleotide, 2′-OMe nucleotide, 2′-O-MOE nucleotide, inverted abasic nucleotide or a 3′->3′ internucleoside linkage.

141. The ωRNA of any one of paragraphs 132-140, wherein the 3′ end modification comprises:
i. a modification of one or more of the last 1-7 nucleotides, wherein the modification is a modified internucleoside linkages (e.g., PS and/or MMI linkage), inverted abasic nucleotide, a 3′->3′ internucleoside linkage, 2′-OMe, 2′-O-MOE, 2′-F, LNA, or a combination thereof;
ii. a modification to the last nucleotide with 2′-OMe, 2′-O-MOE, 2′-F, LNA, or combinations thereof, and an optional one or two modified internucleoside linkages (e.g., PS and/or MMI linkage) to the next nucleotide;
iii. a modification to the last and/or second to last nucleotide with 2′-OMe, 2′-O-MOE, 2′-F, LNA, or combinations thereof, and optionally one or more modified internucleoside linkages (e.g., PS and/or MMI linkage); iv. a modification to the last, second to last, and/or third to last nucleotides with 2′-OMe, 2′-O-moe, 2′-F, or combinations thereof, and optionally one or more modified internucleoside linkages (e.g., PS and/or MMI linkage);
v. a modification to the last, second to last, third to last, and/or fourth to last nucleotides with 2′-OMe, 2′-O-MOE, 2′-F, LNA or combinations thereof, and optionally one or more modified internucleoside linkages (e.g., PS and/or MMI linkage); or vi. a modification to the last, second to last, third to last, fourth to last, and/or fifth to last nucleotides with 2′-OMe, 2′-O-MOE, 2′-F, LNA, or combinations thereof, and optionally one or more modified internucleoside linkages (e.g., PS and/or MMI linkage).

142. The ωRNA of any one of paragraphs 132-141, wherein the 3′ end modification comprises any one or more of:
(i) a 2′-OMe modified nucleotide at the last position, three consecutive 2′-O-MOE modified nucleotides immediately 5′ to the 2′-OMe modified nucleotide, and three consecutive PS linkages between the last three nucleotides;
(ii) five consecutive 2′-OMe modified nucleotides from the 3′ end of the 3′ terminus, and three PS linkages between the last three nucleotides;
(iii) an inverted abasic modified nucleotide at the last position;
(iv) an inverted abasic modified nucleotide at the last position, and three consecutive 2′-OMe modified nucleotides at the last three positions
(v) 15 consecutive 2′-OMe modified nucleotides from the 3′ end, five consecutive 2′-F modified nucleotides immediately 5′ to the 2′-OMe modified nucleotides, and three PS linkages between the last three nucleotides;
(vi) alternating 2′-OMe modified nucleotides and 2′-F modified nucleotides at the last 20 nucleotides, and three PS linkages between the last three nucleotides;
(vii) two or three consecutive 2′-OMe modified nucleotides, and three PS linkages between the last three nucleotides;
(viii) one PS linkage between the last and next to last nucleotides; and
(ix) 15 or 20 consecutive 2′-OMe modified nucleotides, and three PS linkages between the last three nucleotides.

143. The ωRNA of any one of paragraphs 132-142, wherein the 5′ end modification comprises any one or more of:
i. one modified nucleotide;
ii. two modified nucleotides;
iii. three modified nucleotides;
iv. four modified nucleotides;
v. five modified nucleotides;
vi. six modified nucleotides; and
vii. seven modified nucleotides.

144. The ωRNA of any one paragraphs 132-143, wherein the 5′ end modification comprises any one or more of: a modification of between 1 and 7, between 1 and 5, between 1 and 4, between 1 and 3, or between 1 and 2 nucleotides.

145. The ωRNA of any one of paragraphs 132-144, wherein the 5′ end modification comprises any one or more of:
i. a modified internucleoside linkage (e.g., a phosphorothioate and/or MMI linkage) between nucleotides;
ii. a 2′-OMe nucleotide;
iii. a 2′-O-MOE nucleotide;
iv. a 2′-F nucleotide;
v. a LNA;
vi. a 3′->3′ linkage;
vii. an inverted abasic modified nucleotide;
viii. a deoxyribonucleotide;
ix. an inosine; and
x. combinations of one or more of (i.)-(ix.).

146. The ωRNA of any one of paragraphs 132-145, wherein the 5′ end modification comprises:
i. 1, 2, 3, 4, 5, 6, and/or 7 modified internucleoside (e.g., a phosphorothioate and/or MMI) linkages between nucleotides; or
ii. about 1-2, 1-3, 1-4, 1-5, 1-6, or 1-7 modified internucleoside (e.g., a phosphorothioate and/or MMI) linkages between nucleotides.

147 The ωRNA of any one paragraphs 132-146, wherein the 5′ end modification comprises:
i. there is one modified internucleoside (e.g., a phosphorothioate and/or MMI) linkage, and the linkage is between nucleotides 1 and 2;
ii. there are two modified internucleoside (e.g., a phosphorothioate and/or MMI) linkages, and the linkages are between nucleotides 1 and 2, and 2 and 3;
iii. there are modified internucleoside (e.g., a phosphorothioate and/or MMI) linkages between any one or more of nucleotides 1 and 2, 2 and 3, and 3 and 4;
iv. there are modified internucleoside (e.g., a phosphorothioate and/or MMI) linkages between any one or more of nucleotides 1 and 2, 2 and 3, 3 and 4, and 4 and 5;
v. there are modified internucleoside (e.g., a phosphorothioate and/or MMI) linkages between any one or more of nucleotides 1 and 2, 2 and 3, 3 and 4, 4 and 5, and 5 and 6;
vi. there are modified internucleoside (e.g., a phosphorothioate and/or MMI) linkages between any one or more of nucleotides 1 and 2, 2 and 3, 3 and 4, 4 and 5, 5 and 6, and 6 and 7; or
vii. there are modified internucleoside (e.g., a phosphorothioate and/or MMI) linkages between any one or more of nucleotides 1 and 2, 2 and 3, 3 and 4, 4 and 5, 5 and 6, 6 and 7, and 7 and 8.

148. The ωRNA of any one of paragraphs 132-147, wherein the 5′ end modification comprises at least one 2′-OMe, 2′-O-MOE, inverted abasic, or 2′-F modified nucleotide.

149. The ωRNA of any one of paragraphs 46-148, wherein the ωRNA further comprises a guide region capable of hybridizing with a target nucleic acid.

150. The ωRNA of any one of paragraphs 46-149, wherein the ωRNA is hybridized with a second nucleic acid strand.

151. The ωRNA of paragraph 150, wherein the second strand comprises a guide region comprising a sequence capable of hybridizing with a target nucleic acid.

152. The ωRNA of any one of paragraphs 149-151, wherein the guide region is from about 15 to about 30 nucleotides in length.

153. The ωRNA of any one of paragraphs 149-152, wherein the guide region is from about 18 to about 25 nucleotides in length.

154. The ωRNA of any one of paragraphs 149-153, wherein the guide region is from 18, 19, 20, 21, 22 or 23 nucleotides in length.

155. The ωRNA of any one of paragraphs 149-154, wherein the guide region is at 5′-end of the ωRNA.

156. The ωRNA of any one of paragraphs 149-154, wherein the guide region is at 3′-end of the ωRNA.

157. The ωRNA of any one of paragraphs 149-156, wherein the guide region comprises a nucleic acid modification, optionally selected from the group consisting of nucleobase modifications, sugar modifications, inter-sugar linkage modifications (modified internucleoside linkages), conjugates (e.g., ligands), and any combinations thereof.

158. The ωRNA of paragraph 157, wherein said nucleic acid modification is a modified nucleotide selected from the group consisting of 2′-O-methyl(2′-OMe) nucleotides, 2′-fluoro (2′-F) nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl(2′-MOE) nucleotides, 2′-O-allyl nucleotides, 2′-C-allyl ribose nucleotides, 2′-O-N-methylacetamido (2′-O-NMA) nucleotides, a 2′-O-dimethylaminoethoxyethyl(2′-O-DMAEOE) nucleotides, 2′-O-aminopropyl(2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; or a non-natural or modified nucleobase.

159. The ωRNA of any one of paragraphs 149-156, wherein the guide region comprises:
i. a modification of one or more of nucleotides 1-7, wherein the modification is a modified internucleoside (e.g., phosphorothioate and/or MMI) linkage, 3′->3′ internucleoside linkage, inverted abasic nucleotide, 2′-OMe, 2′-O-MOE, 2′-F, 2′-H (a deoxyribonucleotide), an inosine, and/or combinations thereof;
ii. a modification to the first nucleotide of the guide region with 2′-OMe, 2′-O-MOE, 2′-F, 2′-H, an inosine, or combinations thereof, and an optional modified internucleoside (e.g., phosphorothioate and/or MMI) linkage to the next nucleotide;
iii. a modification to the first and/or second nucleotide of the guide region with 2′-OMe, 2′-O-MOE, 2′-F, 2′-H, an inosine, or combinations thereof, and optionally one or more modified internucleoside (e.g., phosphorothioate and/or MMI) linkages;
iv. a modification to the first, second, and/or third nucleotides of the guide region with 2′-OMe, 2′-O-MOE 2′-F, 2′-H, an inosine, or combinations thereof, and optionally one or more modified internucleoside (e.g., phosphorothioate and/or MMI) linkages;
v. a modification to the first, second, third, and/or fourth nucleotides of the guide region with 2′-OMe, 2′-O-MOE, 2′-F, 2′-H, an inosine, or combinations thereof, and optionally one or more modified internucleoside (e.g., phosphorothioate and/or MMI) linkages; or vi. a modification to the first, second, third, fourth, and/or fifth nucleotides of the guide region with 2′-OMe, 2′-O-MOE, 2′-F, 2′-H, an inosine, or combinations thereof, and optionally one or more modified internucleoside (e.g., phosphorothioate and/or MMI) linkages.

160. The ωRNA of any one of paragraphs 149-159, wherein the ωRNA comprises any one or more of:
i. 2′-OMe nucleotides at nucleotides 1, 2, and 3 of the guide region;
ii. 2′-OMe nucleotides at nucleotides 1, 2, and 3 of the guide region and modified internucleoside (e.g., phosphorothioate and/or MMI) linkages between nucleotides 1 and 2, 2 and 3, and 3 and 4 of the guide region;
iii. 2′-OMe nucleotides at nucleotides 1, 2, 3, 4, and 5 of the guide region;
iv. 2′-OMe nucleotides at nucleotides 1, 2, 3, 4, and 5 of the guide region and modified internucleoside (e.g., phosphorothioate and/or MMI) linkages between nucleotides 1 and 2, 2 and 3, 3 and 4, 4 and 5, and 5 and 6 of the guide region;
v. 2′-O-MOE modified nucleotides at nucleotides 1, 2, and 3 of the guide region;
vi. 2′-O-MOE modified nucleotides at nucleotides 1, 2, and 3 of the guide region and modified internucleoside (e.g., phosphorothioate and/or MMI) linkages between nucleotides 1 and 2, 2 and 3, and 3 and 4 of the guide region;
vii. an inverted abasic nucleotide at nucleotide 1 of the guide region;
viii. an inverted abasic nucleotide at nucleotide 1 of the guide region and 2′-OMe nucleotides at nucleotides 1, 2, and 3 of the guide region; and
ix. an inverted abasic nucleotide at nucleotide 1 of the guide region, 2′-OMe nucleotides at nucleotides 1, 2, and 3 of the guide region, and modified internucleoside (e.g., phosphorothioate and/or MMI) linkages between nucleotides 1 and 2, 2 and 3, 3 and 4, 4 and 5, and 5 and 6 of the variable region.

161. The ωRNA of any one of paragraphs 46-160, wherein the ωRNA comprises one of more (e.g., 1, 2, 3, 4, 5, 6, 7, 8, 9, 10 or more including all) modified pyrimidine-A dinucleotide (modified YA site).

162. The ωRNA of any one of paragraphs 46-161, wherein the ωRNA comprises at least one modified YA site in the guide region.

163. The ωRNA of any one paragraphs 46-162, wherein the ωRNA comprises at least one modified YA site in a conserved region of the ωRNA.

164. The ωRNA of any one of paragraphs 46-163, wherein the modified YA site comprises a 2′-fluoro nucleotide, optionally the pyrimidine of the YA site; a 2′-H nucleotide, optionally the pyrimidine of the YA site; a 2′-OMe nucleotide, optionally the pyrimidine of the YA site; a PS linkage between the nucleotides; or a combination thereof.

165. The ωRNA of any one of paragraphs 46-164, wherein the ωRNA comprises:
i. a modified YA site at 1, 2, 3, 4, or 5 guide region YA sites;
ii. a modified YA site at 1, 2, 3, 4, or 5 guide region YA sites, wherein the modification of at least one guide region YA site is different from any 5′ end modification of the ωRNA;
iii. a modified YA site at one or more guide region YA sites that are at or after nucleotide 8 from the 5′ end of the 5′ terminus;
iv. a modified YA site at one or more guide region YA sites that are within nucleotides 5-end, 6-end, 7-end, 8-end, 9-end, or 10-end from the 5′ end of the 5′ terminus;
v. a modified YA site at one or more guide region YA sites that are within 17, 16, 15, 14, 13, 12, 11, 10, or 9 nucleotides of the 3′ terminal nucleotide of the guide region;
vi. a modified YA site at a guide region YA site other than a 5′ end modification; or vii. a modified YA site at a guide region YA site, wherein the modification of the guide region YA site comprises a modification at least one nucleotide located 5′ of the guide region YA site does not comprise.

166. The ωRNA of any one of paragraphs 46-165, wherein the ωRNA comprises:
i. a modified YA site at two or more guide region YA sites that are at or after nucleotide 8 from the 5′-end;
ii. a modified YA site at two or more guide region YA sites that are within nucleotides 5-end, 6-end, 7-end, 8-end, 9-end, or 10-end from the 5′ end;
iii. a modified YA site at two or more guide region YA sites that are within 17, 16, 15, 14, 13, 12, 11, 10, or 9 nucleotides of the 3′ terminal nucleotide of the guide region;
iv. a modified YA site at two or more guide region YA sites other than a 5′ end modification; or
v. a modified YA site at a two or more guide region YA sites, wherein the modifications of the guide region YA sites comprise a modification at least one nucleotide located 5′ of the guide region YA site does not comprise.

167. The ωRNA of any one of paragraphs 46-166, wherein the ωRNA comprises:
i. a modified YA site at three or more guide region YA sites that are at or after nucleotide 8 from the 5′ end of the 5′ terminus;
ii. a modified YA site at three or more guide region YA sites that are within nucleotides 5-end, 6-end, 7-end, 8-end, 9-end, or 10-end from the 5′ end of the 5′ terminus;
iii. a modified YA site at three or more guide region YA sites that are within 17, 16, 15, 14, 13, 12, 11, 10, or 9 nucleotides of the 3′ terminal nucleotide of the guide region;
iv. a modified YA site at three or more guide region YA sites other than a 5′ end modification; or
v. a modified YA site at a three or more guide region YA sites, wherein the modifications of the guide region YA sites comprise a modification at least one nucleotide located 5′ of the guide region YA site does not comprise.

168. The ωRNA of any one of paragraphs 46-167, wherein the ωRNA retains at least 75% of the activity of the parent ωRNA.

169. The ωRNA of any one of paragraphs 46-168, wherein the nuclease is a transposon family nuclease.

170. The RNA of paragraph 169, wherein nuclease is a IS200/IS605 transposon family encoded nuclease or a homolog or ortholog thereof.

171. The ωRNA of paragraph 170, wherein the nuclease is ISC (insertion sequences Cas9-like), TnpB, or a homolog or ortholog thereof.

172. The ωRNA of paragraph 171, wherein the nuclease is IscB nuclease, IsrB nuclease, TnpB nuclease, or a homolog or ortholog thereof, optionally, the nuclease is IscB nuclease or a homolog or ortholog thereof.

173. The ωRNA of any one of paragraphs 46-172, wherein the nuclease is modified nuclease.

174. The ωRNA of any one of paragraphs 46-173, wherein the ωRNA lacks from 1 to about 20 nucleotides present at 3′-end of the conserved portion of the parent ωRNA.

175. A vector comprising the ωRNA of any one of paragraphs 46-174.

176. A composition comprising a ωRNA of any one of paragraphs 46-174 or a polynucleotide encoding the same.

177. The composition of paragraph 176, wherein the composition is in form of a lipid nanoparticles (LNP) or a protein nanoparticles (PNP).

178. The composition of any one of paragraphs 176-177, wherein the composition further comprises a nuclease or a polynucleotide encoding the nuclease.

179. The composition of paragraph 178, wherein the nuclease is a transposon family nuclease.

180 The composition of paragraph 178-179, wherein nuclease is a IS200/IS605 transposon family encoded nuclease or a homolog or ortholog thereof.

181. The composition of paragraph 180, wherein the nuclease is ISC, TnpB, or a homolog or ortholog thereof.

182. The composition of paragraph 181, wherein the nuclease is IscB, IsrB, TnpB nuclease, or a homolog or ortholog thereof, optionally, the nuclease is IscB or a homolog or ortholog thereof.

183. The composition of any one of paragraphs 178-182, wherein the nuclease is a modified nuclease.

184. A pharmaceutical composition comprising an IscB protein of any one of paragraphs 1-31, a fusion protein of any one of paragraphs 32-45, a ωRNA of any one of paragraphs 46-174, and/or a composition of any one of paragraphs 176-183.

185. A DNA and RNA targeting system comprising: (i) a protein effector polypeptide or a polynucleotide encoding the protein effector polypeptide, optionally, the protein effector polypeptide has nuclease (e.g., endonuclease activity); and (ii) a ωRNA of any one of paragraphs 46-174 or a polynucleotide encoding the ωRNA.

186. A DNA and RNA targeting composition comprising: (i) a protein effector polypeptide or a polynucleotide encoding the protein effector polypeptide, optionally, the protein effector polypeptide has nuclease (e.g., endonuclease activity); and (ii) a ωRNA of any one of paragraphs 46-174 or a polynucleotide encoding the ωRNA.

187. The DNA and RNA targeting system of paragraph 185 or the DNA and RNA targeting composition of paragraph 186, wherein the nuclease is a transposon family nuclease.

188. The DNA and RNA targeting system or composition of paragraph 187, wherein nuclease is a IS200/IS605 transposon family encoded nuclease or a homolog or ortholog thereof.

189. The DNA and RNA targeting system or composition of paragraph 188, wherein the nuclease is ISC, TnpB, or a homolog or ortholog thereof.

190. The DNA and RNA targeting system or composition of paragraph 189, wherein the nuclease is IscB, IsrB, TnpB nuclease, or a homolog or ortholog thereof, optionally, the nuclease is IscB or a homolog or ortholog thereof.

191. The DNA and RNA targeting system or composition of any one of paragraphs 185-190, wherein the nuclease is a modified nuclease.

192. The DNA and RNA targeting system or composition of any one of paragraphs 185-191, wherein the protein effector polypeptide comprises a RuvC domain, and/or an HNH domain.

193. The DNA and RNA targeting system or composition of any one of paragraphs 185-192, wherein the protein effector polypeptide comprises: (i) a split Ruv-C nuclease domain comprising RuvC-I, RuvC-II, and RuvC-III subdomains; and/or (ii) an HNH domain.

194. The DNA and RNA targeting system or composition of any one of paragraphs 185-193, wherein the protein effector polypeptide comprises a PLMP domain and optionally a conserved C-terminal Y domain.

195. The DNA and RNA targeting system or composition of any one of paragraphs 192-194, wherein the HNH domain is located in the RuvC domain (e.g., the HNH domain is located between RuvC-II and RuvC-III subdomains).

196. The DNA and RNA targeting system or composition of any one of paragraphs 192-194, wherein the protein effector polypeptide comprises a HNH domain but no RuvC domain.

197. The DNA and RNA targeting system or composition of any one of paragraphs 192-194, wherein the protein effector polypeptide comprises a RuvC (e.g., split RuvC) domain but no HNH domain.

198. The DNA and RNA targeting system or composition of any one of paragraphs 192-197, wherein the protein effector polypeptide further comprises a REC domain.

199. The DNA and RNA targeting system or composition of paragraph 198, wherein the REC domain is downstream from the RuvC or HNH domain.

200. The DNA and RNA targeting system or composition of paragraph 198, wherein the REC domain is upstream from the RuvC or HNH domain.

201. The DNA and RNA targeting system or composition of any one of paragraphs 185-200, wherein the protein effector polypeptide comprises from about 170 to about 1000 amino acids.

202. The DNA and RNA targeting system or composition of any one of paragraphs 185-201, wherein the ωRNA comprises a guide sequence comprising a nucleotide sequence substantially complementary to at least a portion of a target nucleic acid (target sequence)

203. The DNA and RNA targeting system or composition of paragraph 202, wherein the target nucleic acid comprises a target adjacent motif (TAM) sequence 3′ of the target sequence.

204. The DNA and RNA targeting system of paragraph 203, wherein the TAM comprises the sequence NAC, where N is A, C, G, or T.

205. The DNA and RNA targeting system or composition of any one of paragraphs 202-204, wherein the target nucleic acid is DNA.

206 The DNA and RNA targeting system or composition of any one of paragraphs 185-205 further comprising a functional domain associated with the protein effector polypeptide.

207. The DNA and RNA targeting system or composition of paragraph 206, wherein the functional domain has transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, detectable activity, or any combination thereof.

208. The DNA and RNA targeting system or composition of any one of paragraphs 185-207, further comprising a serine or tyrosine recombinase.

209. The DNA and RNA targeting system or composition of any one of paragraphs 185-208, further comprising a homologous recombination donor template comprising a donor sequence for insertion into a target polynucleotide.

210. An engineered cell comprising a DNA and RNA targeting system or composition of any one of paragraphs 185-209.

211. A complex comprising the fusion protein of any one of paragraphs 32-45, and a guide RNA bound to the IscB protein.

212. A complex comprising the fusion protein of any one of paragraphs 32-45, and a guide RNA comprising the ωRNA of any one of paragraphs 46-174 bound to the IscB protein.

213. The complex of paragraph 211 or 212, wherein the guide RNA is about 15-100 nucleotides long and comprises a sequence of at least 10 contiguous nucleotides that is complementary to a target sequence.

214. The complex of any one of paragraphs 211-213, wherein the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long.

215. The complex of any one of paragraphs 211-214, wherein the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

216. The complex of any one of paragraphs 211-215, wherein the target sequence is a DNA sequence.

217. The complex of paragraph 216, wherein the target sequence is a sequence in the genome of a mammal.

218. The complex of paragraph 217, wherein the target sequence is a sequence in the genome of a human.

219. The complex of any one of paragraphs 211-218, wherein the target sequence comprises a sequence associated with a disease or disorder.

220. The complex of paragraph 219, wherein the target sequence comprises a point mutation associated with a disease or disorder.

221. The complex of paragraph 220, wherein the complex edits a point mutation in the target sequence.

222. The complex of paragraph 221, wherein the point mutation is located between about 10 to about 20 nucleotides upstream of the TAM in the target sequence.

223. The complex of paragraph 221 or 222, wherein the target sequence comprises a T to C point mutation.

224. The complex of paragraph 223, wherein the complex deaminates the target C point mutation, and wherein the deamination results in a sequence that is not associated with a disease or disorder.

225. The complex of paragraph 224, wherein the target C point mutation is present in the DNA strand that is not complementary to the guide RNA.

226. The complex of paragraph 221 or 222, wherein the target sequence comprises a G to A point mutation.

227. The complex of paragraph 226, wherein the complex deaminates the target A point mutation, and wherein the deamination results in a sequence that is not associated with a disease or disorder.

228. The complex of paragraph 227, wherein the target A point mutation is present in the DNA strand that is not complementary to the guide RNA.

229. The complex of any one of paragraphs 211-228, wherein the complex exhibits increased deamination efficiency of a point mutation in a target sequence that does not comprise the canonical TAM (5′-ATAAA-3′) at its 3′ end as compared to the deamination efficiency of a complex comprising IscB protein as provided by SEQ ID NO: 2.

230. The complex of paragraph 229, wherein the complex exhibits increased deamination efficiency of a point mutation in a target sequence having a 3′ end that is not directly adjacent to the canonical TAM sequence (5′-ATAAA-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the deamination efficiency of complex comprising the IscB protein as provided by SEQ ID NO: 2 on the same target sequence.

231. The complex of any one of paragraphs 224-230, wherein a deamination activity is measured using a deamination assay, PCR, or sequencing.

232. The complex of any one of paragraphs 211-231, wherein the complex produces fewer indels in a target sequence that does not comprise the canonical TAM (5′-ATAAA-3′) at its 3′ end as compared to the amount of indels produced by a complex comprising IscB protein as provided by SEQ ID NO: 2.

233. The complex of paragraph 232, wherein the complex produces fewer indels in a target sequence having a 3′ end that is not directly adjacent to the canonical TAM sequence (5′-ATAAA-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold lower as compared to the amount of indels produced by a complex comprising IscB protein as provided by SEQ ID NO: 2 on the same target sequence.

234. The complex of any one of paragraphs 232-233, wherein indels are measured using high-throughput sequencing.

235. The complex of any one of paragraphs 211-234, wherein the complex exhibits a decreased off-target activity as compared to the off-target activity of a complex comprising IscB protein as provided by SEQ ID NO: 2.

236. The complex of paragraph 235, wherein the off-target activity of the complex is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold decreased as compared to the off-target activity of a complex comprising IscB protein as provided by SEQ ID NO: 2.

237. The complex of any one of paragraphs 211-236, wherein the target sequence is in the genome of an organism.

238. The complex of paragraph 237, wherein the organism is a prokaryote.

239 The complex of paragraph 237, wherein the prokaryote is a bacterium.

240. The complex of paragraph 237, wherein the organism is a eukaryote.

241. The complex of paragraph 237, wherein the organism is a plant or fungus.

242. The complex of paragraph 237, wherein the organism is a vertebrate.

243. The complex of paragraph 242, wherein the vertebrate is a mammal.

244. The complex of paragraph 243, wherein the mammal is a human.

245. The complex of paragraph 237, wherein the organism is a cell.

246. The complex of paragraph 245, wherein the cell is a human cell.

247. A method comprising contacting a nucleic acid with the fusion protein of any one of paragraphs 32-45, and with a guide RNA, wherein the guide RNA is about 15-100 nucleotides long and comprises a sequence of at least 10 contiguous nucleotides that is complementary to a target sequence.

248. A method comprising contacting a cell with the fusion protein of any one of paragraphs 32-45, and with a guide RNA, wherein the guide RNA is about 15-100 nucleotides long and comprises a sequence of at least 10 contiguous nucleotides that is complementary to a target sequence.

249. A method comprising contacting a nucleic acid with the fusion protein of any one of paragraphs 32-45, and with a guide RNA comprising the ωRNA of any one of paragraphs 46-174.

250. A method comprising contacting a cell with the fusion protein of any one of paragraphs 32-45, and with a guide RNA comprising the ωRNA of any one of paragraphs 46-174.

251. A method comprising contacting a nucleic acid with the complex of any one of paragraphs 211-246.

252. A method comprising contacting a cell with the complex of any one of paragraphs 211-246.

253. The method of any one of paragraphs 247-252, wherein the contacting is performed in vitro.

254. The method of any one of paragraphs 247-253, wherein the contacting is performed in vivo.

255. A method comprising administering to a subject the fusion protein of any one of paragraphs 32-45, and a guide RNA, wherein the guide RNA is about 15-100 nucleotides long and comprises a sequence of at least 10 contiguous nucleotides that is complementary to a target sequence.

256. A method comprising administering to a subject the fusion protein of any one of paragraphs 32-45, and a guide RNA comprising the ωRNA of any one of paragraphs 46-174.

257. A method comprising administering to a subject the complex of any one of paragraphs 211-246 or the DNA and RNA targeting system or composition of any one of paragraphs 185-209.

258. The method of any one of paragraphs 247-257, wherein the target sequence of the nucleic acid is a DNA sequence.

259. The method of any one of paragraphs 247-258, wherein the 3′ end of the target sequence is not immediately adjacent to the canonical TAM sequence (5′-ATAAA-3′).

260. The method of any one of paragraphs 247-259 wherein the target sequence comprises a sequence associated with a disease or disorder.

261. The method of paragraph 260, wherein the target DNA sequence comprises a point mutation associated with a disease or disorder.

262. The method of paragraph 261, wherein the activity of the fusion protein, or the activity of the complex, results in a correction of the point mutation.

263. The method of any one of paragraphs 259-262, wherein the target DNA sequence comprises a T to C point mutation associated with a disease or disorder, and wherein the deamination of the mutant C base results in a sequence that is not associated with a disease or disorder.

264. The method of paragraph 263, wherein the target DNA sequence encodes a protein and wherein the point mutation is in a codon and results in a change in the amino acid encoded by the mutant codon as compared to the wild-type codon.

265. The method of paragraph 263 or 264, wherein the deamination of the mutant C results in a change of the amino acid encoded by the mutant codon.

266. The method of paragraph 263, wherein the deamination of the mutant C results in the codon encoding the wild-type amino acid.

267. The method of any one of paragraphs 261-262, wherein the target DNA sequence comprises a G to A point mutation associated with a disease or disorder, and wherein the deamination of the mutant A base results in a sequence that is not associated with a disease or disorder.

268 The method of paragraph 267, wherein the target DNA sequence encodes a protein and wherein the point mutation is in a codon and results in a change in the amino acid encoded by the mutant codon as compared to the wild-type codon.

269. The method of paragraph 268, wherein the deamination of the mutant A results in a change of the amino acid encoded by the mutant codon.

270. The method of paragraph 267, wherein the deamination of the mutant A results in the codon encoding the wild-type amino acid.

271. The method of any one of paragraphs 247-270, wherein the contacting is in vivo in a subject.

272. The method of paragraph 271, wherein the subject has or has been diagnosed with a disease or disorder.

273. The method of paragraph 271 or 272, wherein the disease or disorder is a proliferative disease, a genetic disease, a neoplastic disease, a metabolic disease, or a lysosomal storage disease.

274. A kit comprising a nucleic acid construct, comprising:
(a) a nucleic acid sequence encoding the fusion protein of any one of paragraphs 32-45; and (b) a heterologous promoter that drives expression of the sequence of (a).

275. A kit comprising a nucleic acid construct, comprising:
(a) a nucleic acid sequence encoding the complex of any one of paragraphs 211-246; and
(b) a heterologous promoter that drives expression of the sequence of (a).

276. The kit of 274 or 275, further comprising an expression construct encoding a guide RNA backbone, wherein the construct comprises a cloning site positioned to allow the cloning of a nucleic acid sequence identical or complementary to a target sequence into the guide RNA backbone.

277. The kit of any one of paragraphs 274-276, further comprising an expression construct encoding a guide RNA comprising the ωRNA of any one of paragraphs 46-174.

278. A polynucleotide encoding the fusion protein of any one of paragraphs 32-45 or the complex of any one of paragraphs 211-246 or the DNA and RNA targeting system or composition of any one of paragraphs 185-209.

279. A vector comprising a polynucleotide of paragraph 278.

280. The vector of paragraph 279, wherein the vector comprises a heterologous promoter driving expression of the polynucleotide encoding the fusion protein or the polynucleotide encoding the complex.

281. A method comprising contacting a cell with the vector of paragraph 279 or 280.

282. The method of paragraph 281, wherein the vector is transfected into the cell.

283. The method of paragraph 282, wherein the vector is transfected into the cell using electroporation, heat shock, or a composition comprising a cationic lipid.

284. A cell comprising the fusion protein of any one of paragraphs 32-45, or a nucleic acid molecule encoding the fusion protein of any one of paragraphs 32-45.

285. A cell comprising the complex of any one of paragraphs 211-246, or a nucleic acid molecule encoding the complex of any one of paragraphs 211-246.

286. A cell comprising the vector of paragraph 279 or 280.

287. A DNA and RNA targeting composition comprising an IscB protein of any one of paragraphs 1-31 and a guide nucleic acid capable of binding with the IscB protein, wherein the guide nucleic acid comprises a guide sequence complementary to target sequence and capable of directing the IscB protein to a target polynucleotide.

288. A DNA and RNA targeting composition comprising an IscB protein of any one of paragraphs 1-31 and a guide nucleic acid comprising the ωRNA of any one of paragraphs 46-174.

289. The composition of paragraph 287 or 288, wherein the guide is from about 10 nucleotides to about 150 nucleotides in length, preferably 12 to 50 nt, more preferably 15 and 45 nt in length.

290. The composition of any one of paragraphs 287-289, wherein the target sequence comprises a target adjacent motif (TAM) sequence 3′ of the target sequence.

291. The composition of any of paragraphs 287-290, wherein the target polynucleotide is DNA.

292. The composition of any of paragraphs 287-291, wherein the guide nucleic acid further comprises an aptamer.

293. The composition of any of paragraphs 287-292, wherein the guide nucleic acid further comprises an extension to add an RNA template.

294. The composition of any of paragraphs 287-293, further comprising a functional domain associated with the IscB protein.

295. The composition of paragraph 294, wherein the functional domain has transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, detectable activity, or any combination thereof.

296. The composition of paragraph 294 or 295, wherein the functional domain is covalently linked with the IscB protein.

297. The composition of paragraph 296, wherein the IscB is fusion protein of any one of paragraphs 32-45

298. The composition of any one of paragraphs 287-297, further comprising a serine or tyrosine recombinase.

299. The composition of any one of paragraphs 287-298, further comprising a homologous recombination donor template comprising a donor sequence for insertion into a target polynucleotide.

300. The composition of any one of paragraphs 287-299, wherein a nuclease domain of the IscB protein is catalytically inactive.

301. The composition of any one of paragraphs 287-300, wherein the nuclease domain has nickase activity or is engineered to have nickase activity.

302. The composition of any one of paragraphs 287-301, wherein the target sequence comprises a TAM sequence of ATR, where R is purine (e.g., G or A).

303. An engineered cell comprising the composition of any one of paragraphs 287-302.

304. A method of modifying a target sequence in a cell, the method comprising administering a composition of any of paragraphs 287-302 to the cell.

305. A method of modifying a target sequence in a cell, the method comprising administering to the cell a composition comprising an IscB protein of any of paragraphs 1 to 31 and/or a guide nucleic acid comprising the ωRNA of any one of paragraphs 46-174, wherein IscB protein and/or the guide nucleic acid are provided via one or more polynucleotides encoding the IscB and/or guide nucleic acid, and wherein the one or more polynucleotides are operably configured to express the IscB/or the guide nucleic acid.

306. The method of paragraph 304 or 305, wherein the modifying comprises cleaving a DNA polynucleotide.

307. The method of any of paragraphs 304-306, wherein the cleaving results in 5′ overhangs.

308. A method of targeting a polynucleotide, comprising contacting a sample that comprises a target polynucleotide with the composition of any one of paragraphs 287-302, or one or more polynucleotides encoding one or more components of the composition of any of paragraphs 287-302.

309. The method of paragraph 308, wherein contacting results in modification of a gene product or modification of the amount or expression of a gene product.

310. The method of paragraph 309, wherein the target sequence of the polynucleotide is a disease-associated target sequence.

311. A composition comprising:
a. the IscB protein of any one of paragraphs 1-31, wherein the IscB protein is catalytically inactive,
b. a nucleotide deaminase associated with or otherwise capable of forming a complex with the IscB protein, and
c. a guide nucleic acid capable of forming a complex with the IscB protein and directing site-specific binding at a target sequence.

312. A composition comprising:
a. the IscB protein of any one of paragraphs 1-31, wherein the IscB protein is catalytically inactive,
b. a nucleotide deaminase associated with or otherwise capable of forming a complex with the IscB protein, and
c. a guide nucleic acid comprising the ωRNA of any one of paragraphs 46-174.

313. The composition of paragraph 311 or 312, wherein the nucleotide deaminase is an adenosine deaminase or a cytidine deaminase.

314. A cell or progeny thereof genetically engineered to express one or more components of the composition of any one of paragraphs 311-313.

315. A method of editing nucleic acids in target polynucleotides comprising delivering the composition of any one of paragraphs 311-313, or one or more polynucleotides encoding one or more components of the composition, to a cell or population of cells comprising the target sequence.

316. The method of paragraph 315, wherein the target polynucleotides are target sequences within genomic DNA.

317. The method of paragraph 315 or 316, wherein the target polynucleotide is edited at one or more bases to introduce a G->A or C->T mutation.

318. An isolated cell or progeny thereof comprising one or more base edits made using the method of any one of paragraphs 315-317.

319. A composition comprising:
a. the IscB protein of any one of paragraphs 1-31, wherein the IscB is catalytically inactive,
b. a reverse transcriptase associated with or otherwise capable of forming a complex with the IscB protein, and
c. a guide nucleic acid capable of forming a complex with the IscB protein and directing site-specific binding of the IscB to a target sequence, and wherein the guide nucleic further comprises a donor sequence for insertion into the target sequence.

320. A composition comprising:
a. the IscB protein of any one of paragraphs 1-31, wherein the IscB is catalytically inactive,
b. a reverse transcriptase associated with or otherwise capable of forming a complex with the IscB protein, and
c. a guide nucleic acid comprising the ωRNA of any one of paragraphs 46-174.

321. A method of modifying target polynucleotides comprising delivering the composition of paragraph 319 or 320, or one or more polynucleotides encoding one or more components of the composition to a cell, wherein the complex directs the reverse transcriptase to the target sequence and the reverse transcriptase facilitates insertion of the donor sequence from the guide nucleic acid into the target sequence

322. The method of paragraph 321, wherein insertion of the donor sequence: introduces one or more base edits; corrects or introduces a premature stop codon; disrupts a splice site; inserts or restores a splice site; inserts a gene or gene fragment at one or both alleles of the target polynucleotide; or a combination thereof.

323. An isolated cell or progeny thereof comprising the modifications made using the method of paragraph 321 or 322.

324. A composition comprising:
a. the IscB protein of any one of paragraphs 1-31;
b. a non-LTR retrotransposon protein associated with or otherwise capable of forming a complex with the IscB protein;
c. guide nucleic acid capable of forming a complex with the IscB protein and directing site-specific binding to a target sequence; and
d. a donor construct comprising a donor polynucleotide for insertion to the target sequence and located between two binding elements capable of forming a complex with the non-LTR retrotransposon protein.

325. A composition comprising:
a. the IscB protein of any one of paragraphs 1-31;
b. a non-LTR retrotransposon protein associated with or otherwise capable of forming a complex with the IscB protein;
c. a guide nucleic acid comprising the ωRNA of any one of paragraphs 46-174; and
d. a donor construct comprising a donor polynucleotide for insertion to the target sequence and located between two binding elements capable of forming a complex with the non-LTR retrotransposon protein.

326. The composition of paragraph 324 or 325, wherein the IscB protein is fused to the N-terminus of the non-LTR retrotransposon protein.

327. The composition of any one of paragraphs 324-326, wherein the IscB protein is engineered to have nickase activity.

328. The composition of paragraph 327, wherein the guide nucleic acid directs the fusion protein to a target sequence 5′ of the targeted insertion site, and wherein the IscB protein generates a double-strand break at the targeted insertion site.

329. The composition of paragraph 327, wherein the guide nucleic acid directs the fusion protein to a target sequence 3′ of the targeted insertion site, and wherein the IscB protein generates a double-strand break at the targeted insertion site.

330. The composition of any one of paragraphs 324-329, wherein the donor polynucleotide further comprises a polymerase processing element to facilitate 3′ end processing of the donor polynucleotide sequence.

331. The composition of any one of paragraphs 324-330, wherein the donor polynucleotide further comprises a homology region to the target sequence on the 5′ end of the donor construct, the 3′ end of the donor construct, or both.

332. The composition of any one of paragraphs 324-331, wherein the homology region is from 8 to 25 base pairs.

333. A method of modifying target polynucleotides comprising delivering the composition of any one of paragraphs 324-332, or one or more polynucleotides encoding one or more components of the composition to a cell, wherein the complex directs the non-LTR retrotransposon protein to the target sequence and the non-LTR retrotransposon protein facilitates insertion of the donor polynucleotide sequence from the donor construct into the target polynucleotide.

334. The method of paragraph 333, wherein insertion of the donor sequence: introduces one or more base edits; corrects or introduces a premature stop codon; disrupts a splice site; inserts or restores a splice site; inserts a gene or gene fragment at one or both alleles of the target polynucleotide; or a combination thereof.

335. An isolated cell or progeny thereof comprising the modifications made using the method of paragraph 333 or 334.

336. A method of modifying a target nucleic acid, the method comprising contacting a DNA and RNA targeting system or composition of any one of paragraphs 185-209 or 287-302.

337. The method of paragraph 336, wherein the target nucleic acid is in a cell and the method comprises administering to the cell a DNA and RNA targeting system or composition of any one of paragraphs 185-209 or 287-302.

338. The method of any one of paragraphs 336-337, wherein said modifying the target nucleic acid comprises cleaving the target nucleic acid.

339. The method of paragraph 338, wherein said cleaving the target nucleic acid forms at least one 5′-overhang.

340. The method of paragraph 338 or 339, wherein said modifying the target nucleic acid comprises deletion, insertion or change of a nucleotide in the target nucleic acid.

341. The method of any one of paragraphs 336-340, wherein said modifying the target nucleic acid comprises editing (e.g., changing) one or more nucleobases; correcting or introducing a premature stop codon, disrupting a splice site; inserting or restoring a splice site, inserting a gene or gene fragment into the target nucleic acid, or a combination thereof.

342 The method of any one of paragraphs 336-341, wherein the target nucleic acid is DNA.

343. The method of paragraph 342, wherein the target nucleic acid is genomic DNA, episomal DNA, viral DNA, or bacterial DNA.

344. The method of paragraph 343, wherein the target nucleic acid is double-stranded DNA. 345. The method of paragraph 343, wherein the target nucleic acid is single-stranded DNA.

346. The method of any one of paragraphs 336-341, wherein the target nucleic acid is RNA.

347. The method of paragraph 346, wherein the target nucleic acid is double-stranded RNA.

348. The method of paragraph 346, wherein the target nucleic acid is single-stranded RNA.

349. The method of paragraph 346, wherein the target nucleic acid is RNA selected from the group consisting of messenger RNA (mRNA), pre-mRNA, ribosomal RNA (rRNA), transfer RNA (tRNA), micro-RNA (miRNA), small nuclear RNA (snRNA), small nucleolar RNA (snoRNA), non-coding RNA (ncRNA), long non-coding RNA (lncRNA), and small cytoplasmatic RNA (scRNA).

350. The method of any one of paragraphs 336-349, wherein the target nucleic acid is a disease-associated nucleic acid sequence.

351. An isolated nucleic acid modified by a method of anyone of paragraphs 336-349. An isolated cell or progeny thereof comprising a nucleic acid modified by a method of anyone of paragraphs 336-349.

### EXAMPLES

The technology described herein is further illustrated by the following examples which in no way should be construed as being further limiting.

### Example 1—In Vitro Cleavage of Target Library to Determine TAM Preference

The TAM preference of IscB variants was determined by deep sequencing of DNA plasmids that were cleaved by nucleases produced in a reconstituted in vitro protein expression system (PUREXPRESS, NEB). Nuclease variants were transcribed and translated from a PCR fragment containing a T7 promoter, an E. coli codon optimized gene, and a T7 terminator; a transcribed @ RNA was included to generate a ribonucleoprotein complex with the translated nuclease. The @ RNA served as a guide to target a pool of plasmids containing randomly-generated 8-nucleotide TAM sequences on the 3′ end of a target sequence.

An 8N plasmid library, PCR product of a nuclease variant, and ωRNA was incubated in a PUREXPRESS reaction for 4 hours at 37° C. The reaction was enzymatically stopped with RNase & Proteinase K treatment, and the DNA recovered with a DNA clean-up kit (AMPURE XP beads). Successfully cleaved plasmids resulted in linear DNA, to which an adapter was ligated (NEB DNA Library Prep Kit for ILLUMINA). Uncleaved plasmids were not adapter ligated. Those sequences containing adapters were then PCR amplified one primer specific to the target plasmid library backbone and one primer specific to the adapter, thus amplifying only for cleaved DNA. A subsequent PCR amplification was carried out to introduce unique ILLUMINA i5 and i7 indexes for NGS sequencing. TAM sequences were extracted from the sequencing data and an enrichment score was calculated for each TAM using Simple Enrichment Analysis (SEA). A position weight matrix was generated based on enrichment score, and logos generated using Sensitive, Thorough, Rapid, Enriched Motif Elicitation (STREME).

### Example 2—Genomic DNA Cleavage Assay in HEK293FT Cells

To evaluate the effect of IscB point mutations, the corresponding nuclease- and guide-expression plasmids were co-transfected into HEK293-FT cells using LIPOFECTAMINE 2000 with technical triplicates. Cells were harvested 72 hours post-transfection, and the genomic DNA was extracted by QUICKEXTRACT solution. Genomic region containing the intended target site of IscB was amplified with target-specific primer pairs using Phusion U DNA polymerase (THERMO) and barcoded with standard ILLUMINA adapter primers in the 2^{nd} round PCR for multiplexed sequencing on a NEXTSEQ 2000 instrument (ILLUMINA). Sequencing data was processed and analyzed by in-house pipeline to measure the nuclease-induced, guide RNA dependent indels (i.e. editing efficiency). The editing efficiency of technical triplicates for a given nuclease variant were averaged (Table 20). Samples with less than 10,000 sequencing reads were omitted from analysis.

TABLE 20
Activity of IscB and variant on genomic targets with ATAAA and GTAAA TAM.
The effect of the H478N point mutation was evaluated by measuring the editing
efficiency in HEK293-FT cells over forty-nine genomic targets with either
ATAAA or GTAAA TAM. Values represent % of sequencing reads with insertions or
deletions (indels). The additionvof H478N increased average indel activity on
GTAAA targets while decreasing activity on ATAAA targets.

| Target | | | IscB w/H478N | |
|---|---|---|---|---|
| Name | TAM | IscB | Mutant | Target Sequence (5′-3′) |
| EMX1-1 | ATAAA | 2.625 | 1.973333333 | ACTACAGTGGTGCCTGGAAA |
| | | | | (SEQ ID NO: 166) |
| EMX1-2 | ATAAA | 2.47666667 | 0.016666667 | CCCCCTTCCCTATGGGAATA |
| | | | | (SEQ ID NO: 167) |
| EMX1-3 | ATAAA | 1.09 | 0.01 | GTCCAGGCACTGCTCTTGAT |
| | | | | (SEQ ID NO: 168) |
| EMX1-4 | ATAAA | 2.46333333 | 0 | GAGCACCCCCTGTCATCCGA |
| | | | | (SEQ ID NO: 169) |
| EMX1-5 | ATAAA | 1.61666667 | 0.003333333 | TTGGTGTTGTGACTTCATTA |
| | | | | (SEQ ID NO: 170) |
| HPRT-1 | ATAAA | 4.715 | 3.175 | gcaattataagccatttcac |
| | | | | (SEQ ID NO: 171) |
| HPRT-2 | ATAAA | 0.655 | 0.385 | aatcataattcatcatttag |
| | | | | (SEQ ID NO: 172) |
| HPRT-3 | ATAAA | 0.625 | 0.49 | actcatttttcaaaacacgc |
| | | | | (SEQ ID NO: 173) |
| HPRT-4 | ATAAA | 2.49 | 2.055 | tgatggttcccattagtcac |
| | | | | (SEQ ID NO: 174) |
| HPRT-5 | ATAAA | 0.715 | 0.44 | ttgcattattatttcagatg |
| | | | | (SEQ ID NO: 175) |
| HPRT-6 | ATAAA | 3.725 | 3.175 | aatgtggtcataagtaagaa |
| | | | | (SEQ ID NO: 176) |
| HPRT-7 | ATAAA | 3.17 | 3.15 | ggaaatacaatccctaaact |
| | | | | (SEQ ID NO: 177) |
| HPRT-8 | ATAAA | 2.115 | 1.11 | AATCTTCCTCAACCATGTTC |
| | | | | (SEQ ID NO: 178) |
| HPRT-9 | ATAAA | 8.93666667 | 7.336666667 | ACTAATGACACAAACATGCT |
| | | | | (SEQ ID NO: 179) |
| HPRT-10 | ATAAA | 1.60666667 | 0.873333333 | ATTTCTAAAGTACAAAACAG |
| | | | | (SEQ ID NO: 180) |
| HPRT-11 | ATAAA | 4.6 | 5.013333333 | TCTATAGGCTCATAGTGCAA |
| | | | | (SEQ ID NO: 181) |
| HPRT-12 | ATAAA | 1.69 | 1.116666667 | TGAACTCATATCTGTAAGAA |
| | | | | (SEQ ID NO: 182) |
| HPRT-13 | ATAAA | 0.11666667 | 0.113333333 | TTCTATTCTTTCCTGCATAT |
| | | | | (SEQ ID NO: 183) |
| HPRT-14 | ATAAA | 1.84666667 | 1.593333333 | TCCCTTTTAAATGCAAATCA |
| | | | | (SEQ ID NO: 184) |
| HPRT-15 | ATAAA | 0.14333333 | 0.036666667 | AGGATTTTTAAATTGAATTC |
| | | | | (SEQ ID NO: 185) |
| VEGFA-1 | ATAAA | 11.18 | 0.036666667 | TTTAAGAAAAAAGAAGAGGG |
| | | | | (SEQ ID NO: 186) |
| VEGFA-2 | ATAAA | 4.06333333 | 0.033333333 | GTGGGGAATGGCAAGCAAAA |
| | | | | (SEQ ID NO: 187) |
| VEGFA-4 | ATAAA | 4.69666667 | 0.04 | CAGGTCTTAGTAAAAAACAA |
| | | | | (SEQ ID NO: 188) |
| VEGFA-5 | ATAAA | 1.06 | 0.016666667 | AGGGGGCCCTGAGCCAGGAA |
| | | | | (SEQ ID NO: 189) |
| GTAAA- | GTAAA | 0.16 | 0.823333333 | AAACGTCCCTGGACAGGCTG |
| AAVS1-1 | | | | (SEQ ID NO: 190) |
| GTAAA- | GTAAA | 0.62333333 | 2.163333333 | GGGGTCAGCAAACTTTCTCT |
| AAVS1-3 | | | | (SEQ ID NO: 191) |
| GTAAA- | GTAAA | 0.27 | 0.66 | ACTGAATTGGGGTTAAAATG |
| AAVS1-4 | | | | (SEQ ID NO: 192) |
| GTAAA- | GTAAA | 0.17 | 1.463333333 | GCTATTGCTTCAACTTTTCT |
| AAVS1-5 | | | | (SEQ ID NO: 193) |
| GTAAA- | GTAAA | 0.43333333 | 0.853333333 | CTACTGGCCTTATCTCACAG |
| AAVS1-6 | | | | (SEQ ID NO: 194) |
| GTAAA- | GTAAA | 0.09333333 | 1.556666667 | GGCTAATCTTCTTCCCGTCT |
| EMX1-1 | | | | (SEQ ID NO: 195) |
| GTAAA- | GTAAA | 7.62666667 | 18.95 | GCCTGACTCTGCAAAGCCAA |
| EMX1-6 | | | | (SEQ ID NO: 196) |
| GTAAA- | GTAAA | 0.95666667 | 5.3 | TGTGCCTCTCCCTGCAGACG |
| EMX1-8 | | | | (SEQ ID NO: 197) |
| GTAAA- | GTAAA | 4.24666667 | 7.423333333 | TCCCCTCATCAAAAATAGGA |
| VEGFA-1 | | | | (SEQ ID NO: 198) |
| GTAAA- | GTAAA | 2.98 | 8.783333333 | GTCCTTAAGAGTACATTGTT |
| VEGFA-2 | | | | (SEQ ID NO: 199) |
| GTAAA- | GTAAA | 0.2 | 1.95 | ACCTGAAAGAGCAGGTCTTA |
| VEGFA-3 | | | | (SEQ ID NO: 200) |
| GTAAA- | GTAAA | 1.145 | 2.383333333 | ACAAGGTCTTTGTACAGTTA |
| VEGFA-4 | | | | (SEQ ID NO: 201) |
| GTAAA- | GTAAA | 0.94666667 | 4.676666667 | CAAGCCATTTCTCCAAAAGG |
| VEGFA-5 | | | | (SEQ ID NO: 202) |
| GTAAA- | GTAAA | 0.68333333 | 1.01 | CAAGGGGTTGGTAACTGAGG |
| VEGFA-7 | | | | (SEQ ID NO: 203) |
| GTAAA- | GTAAA | 1.74666667 | 3.126666667 | CAAGGCCGCACAGCTAGTAA |
| VEGFA-8 | | | | (SEQ ID NO: 204) |
| GTAAA- | GTAAA | 3.84666667 | 4.005 | ACGTGGCAAAAAAAGATGGA |
| HPRT-1 | | | | (SEQ ID NO: 205) |
| GTAAA- | GTAAA | 0.05333333 | 0.07 | GGCTAATTTTTGTATTTTTA |
| HPRT-2 | | | | (SEQ ID NO: 206) |
| GTAAA- | GTAAA | 3.25333333 | 10.895 | AACTAATAGTGTTCTTATAT |
| HPRT-3 | | | | (SEQ ID NO: 207) |
| GTAAA- | GTAAA | 8.15 | 15.315 | GAGTCCTATTGACATCGCCA |
| HPRT-4 | | | | (SEQ ID NO: 208) |
| GTAAA- | GTAAA | 2.28666667 | 6.435 | GGCGGATTGTTGTTTAACTT |
| HPRT-5 | | | | (SEQ ID NO: 209) |
| GTAAA- | GTAAA | 0.26666667 | 0.465 | AGCCGTGTTATTTTAGCATA |
| HPRT-6 | | | | (SEQ ID NO: 210) |
| GTAAA- | GTAAA | 0.04666667 | 0.793333333 | AACAGAAGTAAAAATGTTTT |
| HPRT-7 | | | | (SEQ ID NO: 211) |
| GTAAA- | GTAAA | 0.87666667 | 1.4 | GATCAAATGAGATAACAGAA |
| HPRT-8 | | | | (SEQ ID NO: 212) |
| GTAAA- | GTAAA | 0.03333333 | 0.075 | TAGCAACCCACCACTTGTGG |
| HPRT-9 | | | | (SEQ ID NO: 213) |
| GTAAA- | GTAAA | 0.04 | 0.23 | GGACCTGCACTTCTTCATCT |
| HPRT-10 | | | | (SEQ ID NO: 214) |

TABLE 21
Modifications and TAM variants

| Name | Mutation | TAM | Logo |
|---|---|---|---|
| WT | — | ATAAA | [symbol] |
| Mut1 | H478N | RTAAA | [symbol] |
| Mut2 | H479N | RTAAA | [symbol] |
| Mut3 | I564R | ATRAA | [symbol] |
| Mut4 | I564K | ATRMM | [symbol] |
| Mut5 | I564H | ATRA | [symbol] |
| Mut6 | I564N | ATRAA | [symbol] |
| Mut7 | I564Q | ATRRA | [symbol] |
| Mut8 | I564C | ATRRA | [symbol] |
| Mut9 | I564M | ATRRA | [symbol] |
| Mut10 | I564T | ATARA | [symbol] |
| Mut11 | I564D | ATRA | [symbol] |
| Mut12 | T565R | ATRRAC | [symbol] |
| Mut13 | T565K | ATAAAC | [symbol] |
| Mut14 | T565H | ATRRA | [symbol] |
| Mut15 | T565C | ATARA | [symbol] |
| Mut16 | T565D | ATARA | [symbol] |
| Mut17 | T565G | ATRRA | [symbol] |
| Mut18 | H478C | RTAAA | [symbol] |
| Mut19 | H478P | RTAAA | [symbol] |
| Mut20 | H479C | AYAAA | [symbol] |
| Mut21 | H479M | AYAAA | [symbol] |

TABLE 22
TAM Variants, Swap sequences and regions. (Table discloses SEQ ID
NOS 17-47, 11-15, 48, 16 and 49-73, respectively, in order of appearance)

| Name | Swap Sequence | Swap region (wt) | TAM | Logo |
|---|---|---|---|---|
| SWP6 | QHCHGA | Q563-K568 | ATRAA | [symbol] |
| SWP7 | NSHYGQ | Q563-K568 | ATARM | [symbol] |
| SWP9 | QHCHGN | Q563-K568 | ATRRA | [symbol] |
| SWP11 | QQNKGD | Q563-K568 | ATRAA | [symbol] |
| SWP15 | THAKS | Q563-K568 | ATRAA | [symbol] |
| SWP16 | QKNNGV | Q563-K568 | ATRAM | [symbol] |
| SWP17 | VLNKGA | Q563-K568 | ATRAA | [symbol] |
| SWP20 | SSGAGK | Q563-K568 | ATRRM | [symbol] |
| SWP23 | NSNGGA | Q563-K568 | ATARM | [symbol] |
| SWP30 | IKNRGR | Q563-K568 | ATRAM | [symbol] |
| SWP31 | MHNKGK | Q563-K568 | ATRAM | [symbol] |
| SWP38 | VFNYGK | Q563-K568 | ATRAA | [symbol] |
| SWP54 | THNKGA | Q563-K568 | ATRAM | [symbol] |
| SWP56 | AHGYGK | Q563-K568 | ATRMA | [symbol] |
| SWP58 | NKNKGK | Q563-K568 | ATRMM | [symbol] |
| SWP70 | THSYGK | Q563-K568 | ATRA | [symbol] |
| SWP76 | MHSYGR | Q563-K568 | ATRA | [symbol] |
| SWP80 | SQNKGT | Q563-K568 | ATRAA | [symbol] |
| SWP84 | TTGLGK | Q563-K568 | ATANA | [symbol] |
| SWP87 | KTNN | Q563-K568 | ATRR | [symbol] |
| SWP119 | TQNKGR | Q563-K568 | ATRAA | [symbol] |
| SWP124 | THCKGT | Q563-K568 | ATRAA | [symbol] |
| SWP168 | QHTNGR | Q563-K568 | ATRA | [symbol] |
| SWP170 | QRTNGN | Q563-K568 | ATRAA | [symbol] |
| SWP171 | QRTNGS | Q563-K568 | ATRAA | [symbol] |
| SWP177 | QRTRGQ | Q563-K568 | ATRAM | [symbol] |
| SWP182 | QASGGK | Q563-K568 | ATRAA | [symbol] |
| SWP186 | QSSGGK | Q563-K568 | ATRAA | [symbol] |
| SWP188 | QQSNGR | Q563-K568 | ATRRA | [symbol] |
| SWP189 | QHSNGA | Q563-K568 | ATRA | [symbol] |
| SWP197 | QKNRGY | Q563-K568 | ATRMM | [symbol] |
| SWP199 | QTSGGR | Q563-K568 | ATR | [symbol] |
| SWP201 | RSSGGK | Q563-K568 | ATR | [symbol] |
| SWP202 | QQSKGR | Q563-K568 | ATR | [symbol] |
| SWP203 | QQGRGR | Q563-K568 | ATR | [symbol] |
| SWP204 | QQNKGR | Q563-K568 | ATR | [symbol] |
| SWP206 | QLSKGI | Q563-K568 | ATRMM | [symbol] |
| SWP216 | QKDKGA | Q563-K568 | ATR | [symbol] |
| SWP218 | TKNKGT | Q563-K568 | ATRMM | [symbol] |
| SWP244 | GKNGYV | Q563-K568 | ATGAC | [symbol] |
| SWP250 | MSGQHCHGAYLR | M560-R571 | ATRAA | [symbol] |
| SWP251 | LSGNSHYGQYYM | M560-R571 | ATARA | [symbol] |
| SWP253 | MTGQHCHGNYFR | M560-R571 | ATRAA | [symbol] |
| SWP255 | MTGQQNKGDRYF | M560-R571 | ATRAA | [symbol] |
| SWP257 | KSGQLTNGQYFR | M560-R571 | ATRRA | [symbol] |
| SWP258 | LKGNACNGAYFH | M560-R571 | ATRRA | [symbol] |
| SWP259 | SSGTHAKSYFR | M560-R571 | ATRAA | [symbol] |
| SWP260 | LGGQKNNGVYFY | M560-R571 | ATRAM | [symbol] |
| SWP264 | VTGSSGAGKYVR | M560-R571 | ATARM | [symbol] |
| SWP273 | VRSVHSYGKYVR | M560-R571 | ATAA | [symbol] |
| SWP274 | VQGIKNRGRYLH | M560-R571 | ATRAM | [symbol] |
| SWP279 | LRGTSSYGKTVL | M560-R571 | ATAR | [symbol] |
| SWP282 | VKGVFNYGKWVR | M560-R571 | ATRRA | [symbol] |
| SWP284 | VKGKHNDRWI | M560-R571 | ATGAC | [symbol] |
| SWP285 | VKGVQNRGKYIK | M560-R571 | ATRRA | [symbol] |
| SWP287 | IKGSHCNGSRLI | M560-R571 | ATRA | [symbol] |
| SWP290 | SGGVMSYGRYIT | M560-R571 | ATAAAC | [symbol] |
| SWP302 | SKGNKNKGKSIL | M560-R571 | ATGMC | [symbol] |
| SWP305 | AIGTHCKGKRVI | M560-R571 | ATGAA | [symbol] |
| SWP315 | VKGVFNRGTWVR | M560-R571 | ATRRA | [symbol] |
| SWP320 | CKGMHSYGRYVL | M560-R571 | ATRAR | [symbol] |
| SWP363 | IGGTQNKGRYAI | M560-R571 | ATRAA | [symbol] |
| SWP368 | TKGTHCKGTRIM | M560-R571 | ATRA | [symbol] |

Exemplary lead TAM sequences are RTAAA, ATRAA, ATRMM, ATRA, ATRRA, ATARA, ATRRAC, ATAAAC, ATARM, ATRAM, ATRRM, ATRMA, ATANA, ATRR, ATR, ATGAC, ATAA, ATAR, ATGMC, ATGAA, and ATRAR.

### Example 3: Protein Mutagenesis to Improve the On-Target Activity of EboIscB

EboIscB is a RNA-guided endonuclease with low activity in human cells. Previous work by Altae-Tan et al. (“The widespread IS200/IS605 transposon family encodes diverse programmable RNA-guided endonucleases,” Science 2021 October, 374 (6563): 57-65, the contents of which are incorporated herein by reference in their entirety) inserted a REC from Cas9-IID into the EboIscB (i.e. EboIscB-REC), and identified a list of single point mutations to improve the on-target activity. To create a potent nuclease variant, the existing point mutations from Altae-Tan et al. were stacked, and EboIscB-REC variants were generated with 3 or 7 point mutations that synergistically enhanced the on-target activity. However, even with the most potent variant, the activity of EboIscB-REC is still lower than SpCas9. To further enhance the activity, a large panel of variants were evaluated using WT EboIscB-REC as the background. These variants includes: 1) R/K mutations for the residues around the predicted protein-DNA or protein-RNA/DNA R-loop interface; 2) R mutations for the remaining of the EboIscB-REC; 3) Nuclear localization signal (NLS) sequences on both N′ and C′-terminus of the EboIscB-REC expression construct. The work revealed large panel of point mutations/NLS sequences that can enhance the on-target activity of EboIscB-REC in human cells, which allows to further engineer this nuclease on top the existing variant.

### Results

To validate the result of previous invention by Altae-Tan et al., the EboIscB-REC construct (pEDIT071) was generated as previously described, and stacked the top three-point mutations (E409K/D413K/1533R, i.e. pEDIT152) from the mutation list. To comprehensively compare the activity of these constructs in human cells, the editing efficiency of EboIscB constructs were measured over 48 randomly chosen genomic targets in human cells. As expected, inserting the REC domain to EboIscB enhanced the activity for ~4-fold (median editing efficiency from 0.35% to 1.42%), and the addition of three-point mutations further doubled the efficiency (median efficiency 2.76%, Table 23, FIG. 1). The activity of the rest point mutations was screened when stacked on pEDIT152, and resulted in a series of variants containing 4~7 point mutations with further enhanced activity (Table 23, FIG. 1). Collectively, these engineering efforts enhanced the editing efficiency of EboIscB for ~18-folds (median activity from 0.35% to 6.28%).

To further elevate the activity of EboIscB, a comprehensive screening of new variants was performed, including Arg or Lys point mutations and NLS variants. These variants were evaluated in the backbone of pEDIT071 over two genomic targets (Table 25), and reported the normalized activity over the parental construct. Over 100 point mutations enhanced the editing at either or both targets were reported in Table 24, providing ample candidates for further stacking on the existing constructs.

TABLE 23
Comparison of on-target activity of engineered EboIscB constructs.

| | | | | | | | Triple + |
|---|---|---|---|---|---|---|---|
| | | | | | Triple + | Triple + | E137K + |
| | | | | Triple + | E137K + | E137K + | N66H + |
| | | EboIscB + | EboIscB + | E137K + | N66H + | N66H + | A100K + |
| | EboIscB | REC | REC + Triple | T536R | A100K | T536R | T536R |
| Table 23 | (pEDIT070) | (pEDIT071) | (pEDIT152) | (pEDIT416) | (pEDIT417) | (pEDIT418) | (pEDIT420) |
| PT-1 | 0.06 ± 0.06 | 0.27 ± 0.05 | 0.22 ± 0.05 | 0.37 ± 0.13 | 0.59 ± 0.16 | 0.38 ± 0.16 | 0.34 ± 0.05 |
| PT-2 | 0.52 ± 0.03 | 1.81 ± 0.46 | 4.3 ± 0.3 | 6.51 ± 0.3 | 5.42 ± 0.55 | 6.6 ± 0.22 | 6.59 ± 0.33 |
| PT-3 | 0.47 ± 0.06 | 4.55 ± 0.38 | 6.04 ± 0.11 | 6.05 ± 0.54 | 7.93 ± 0.33 | 6.31 ± 0.15 | 7.83 ± 0.98 |
| PT-4 | 0.39 ± 0.09 | 1.64 ± 0.1 | 2.21 ± 0.03 | 2.8 ± 0.09 | 4.19 ± 0.33 | 2.88 ± 0.35 | 4.22 ± 0.19 |
| PT-5 | 0.7 ± 0.05 | 1.52 ± 0.23 | 1.94 ± 0.28 | 2.39 ± 0.26 | 3.91 ± 0.61 | 2.22 ± 0.19 | 4.12 ± 0.22 |
| PT-6 | 0.61 ± 0.08 | 1.07 ± 0.17 | 1.76 ± 0.43 | 5.35 ± 0.55 | 8.27 ± 0.26 | 5.72 ± 1.08 | 8.9 ± 0.96 |
| PT-7 | 1.08 ± 0.09 | 5.61 ± 0.11 | 8.03 ± 0.93 | 12.65 ± 1.87 | 13.44 ± 0.49 | 12 ± 0.6 | 16.23 ± 0.59 |
| PT-8 | 0.06 ± 0.03 | 0.76 ± 0.12 | 3.34 ± 0.12 | 6.2 ± 0.58 | 8.9 ± 0.52 | 5.76 ± 0.37 | 10 ± 0.97 |
| PT-9 | 0.04 ± 0.03 | 0.6 ± 0.11 | 2.48 ± 0.24 | 4.45 ± 0.18 | 6.97 ± 0.41 | 5.35 ± 0.55 | 6.13 ± 0.75 |
| PT-10 | 0.01 ± 0.01 | 1.56 ± 0.32 | 2.72 ± 0.58 | 5.31 ± 0.58 | 6.96 ± 0.9 | 5.3 ± 0.43 | 6.6 ± 0.2 |
| PT-11 | 0.17 ± 0.02 | 0.12 ± 0.01 | 0.29 ± 0.08 | 1.04 ± 0.1 | 0.74 ± 0.04 | 1.48 ± 0.2 | 1.51 ± 0.21 |
| PT-12 | 2.13 ± 0.22 | 2.48 ± 0.56 | 3 ± 0.41 | 3.28 ± 0.5 | 5.18 ± 0.63 | 3.27 ± 0.26 | 3.87 ± 0.25 |
| PT-13 | 0.86 ± 0.03 | 3.22 ± 0.25 | 4.22 ± 0.95 | 4.04 ± 0.29 | 4.33 ± 0.39 | 5.44 ± 0.16 | 5.42 ± 0.17 |
| PT-14 | 2.98 ± 0.17 | 5.77 ± 0.62 | 9.51 ± 1.86 | 14.76 ± 1.27 | 13.82 ± 1.69 | 15.07 ± 2 | 18.48 ± 1.88 |
| PT-15 | 0.21 ± 0.06 | 0.59 ± 0.1 | 1.88 ± 0.06 | 3.17 ± 0.37 | 3.77 ± 0.08 | 4.49 ± 0.65 | 6.48 ± 0.63 |
| PT-16 | 0.84 ± 0.19 | 1.48 ± 0.17 | 1.69 ± 0.11 | 4.24 ± 0.09 | 5.81 ± 0.2 | 4.56 ± 0.37 | 7.2 ± 0.45 |
| PT-17 | 0.49 ± 0.08 | 4.07 ± 0.5 | 10.93 ± 1.25 | 14.43 ± 0.9 | 11.27 ± 0.75 | 13.47 ± 0.25 | 14.89 ± 1.74 |
| PT-18 | 0.07 ± 0.03 | 0.18 ± 0.03 | 0.44 ± 0.1 | 2.4 ± 0.18 | 1.6 ± 0.11 | 2.21 ± 0.45 | 4.45 ± 0.18 |
| PT-19 | 0.22 ± 0.02 | 1.46 ± 0.22 | 2.71 ± 0.05 | 4.34 ± 0.52 | 5.78 ± 0.06 | 5.58 ± 0.32 | 5.7 ± 0.22 |
| PT-20 | 1.59 ± 0.05 | 3.22 ± 0.17 | 5.09 ± 1.07 | 4.97 ± 0.48 | 5.82 ± 0.35 | 5.44 ± 0.56 | 6.43 ± 0.77 |
| PT-21 | 0.68 ± 0.06 | 1.01 ± 0.1 | 1.43 ± 0.28 | 3.11 ± 0.06 | 3.61 ± 0.28 | 3.82 ± 0.58 | 6.8 ± 0.3 |
| PT-22 | 0.22 ± 0.05 | 0.16 ± 0.02 | 0.23 ± 0.07 | 0.15 ± 0.03 | 0.26 ± 0.09 | 0.15 ± 0.02 | 0.19 ± 0.01 |
| PT-23 | 0.87 ± 0.03 | 1.98 ± 0.41 | 2.5 ± 0.7 | 2.48 ± 0.03 | 5.26 ± 0.54 | 2.84 ± 0.14 | 5.23 ± 0.82 |
| PT-24 | 0.01 ± 0.01 | 1.39 ± 0.23 | 3.19 ± 0.34 | 4.78 ± 0.08 | 5.87 ± 0.02 | 5.06 ± 0.33 | 7.36 ± 0.35 |
| PT-25 | 2.26 ± 0.28 | 1.27 ± 0.14 | 1.86 ± 0.16 | 1.7 ± 0.17 | 1.83 ± 0.2 | 1.68 ± 0.34 | 1.75 ± 0.3 |
| PT-26 | 0.04 ± 0.02 | 1.06 ± 0.26 | 1.52 ± 0.07 | 1.53 ± 0.04 | 2.82 ± 0.12 | 1.9 ± 0.22 | 2.25 ± 0.26 |
| PT-27 | 0.2 ± 0.05 | 4.14 ± 0.11 | 8.89 ± 0.46 | 13.04 ± 0.66 | 11.55 ± 0.78 | 13.93 ± 0.23 | 16.1 ± 1.16 |
| PT-28 | 3.55 ± 0.28 | 2.33 ± 0.2 | 5.22 ± 0.44 | 12.39 ± 0.68 | 4.59 ± 0.45 | 13.39 ± 0.96 | 12.81 ± 0.55 |
| PT-29 | 1.33 ± 0.03 | 2.98 ± 0.05 | 4.03 ± 0.62 | 6.12 ± 0.51 | 7.38 ± 0.35 | 6.95 ± 0.18 | 8.08 ± 0.38 |
| PT-30 | 0.2 ± 0.05 | 1.74 ± 0.16 | 2.21 ± 0.19 | 4.88 ± 0.4 | 11.49 ± 0.22 | 5.5 ± 0.64 | 9.43 ± 0.14 |
| PT-31 | 1.42 ± 0.06 | 1.29 ± 0.13 | 3.15 ± 0.17 | 2.96 ± 0.2 | 2.61 ± 0.11 | 2.59 ± 0.46 | 3.24 ± 0.1 |
| PT-32 | 0.84 ± 0.04 | 0.73 ± 0.15 | 2.29 ± 0.4 | 7.23 ± 0.66 | 7.57 ± 0.52 | 6.51 ± 0.34 | 7.59 ± 0.18 |
| PT-33 | 0.23 ± 0.03 | 3.35 ± 0.31 | 7.51 ± 0.79 | 13.06 ± 0.5 | 16.47 ± 1.31 | 15 ± 1.17 | 16.04 ± 1.94 |
| PT-34 | 0.64 ± 0.18 | 2.35 ± 0.06 | 2.88 ± 0.03 | 4.28 ± 0.21 | 10.45 ± 0.81 | 4.13 ± 0.59 | 7.78 ± 0.25 |
| PT-35 | 0.01 ± 0.01 | 0.01 ± 0.01 | 0.03 ± 0.02 | 0.27 ± 0.03 | 0.2 ± 0.02 | 0.34 ± 0.05 | 0.66 ± 0.1 |
| PT-36 | 0.02 ± 0 | 0.35 ± 0.13 | 0.41 ± 0.07 | 0.39 ± 0.11 | 0.85 ± 0.16 | 0.64 ± 0.08 | 0.87 ± 0.13 |
| PT-37 | 0.35 ± 0.1 | 0.58 ± 0.24 | 3.23 ± 0.22 | 7.67 ± 0.41 | 6.17 ± 0.64 | 11.38 ± 0.36 | 12.43 ± 1 |
| PT-38 | 0.41 ± 0.03 | 2.6 ± 0.27 | 6.95 ± 1.23 | 15.65 ± 1.28 | 17.06 ± 0.45 | 15.89 ± 0.9 | 21.49 ± 1.29 |
| PT-39 | 0.04 ± 0.01 | 2.34 ± 0.36 | 4.21 ± 0.43 | 9.36 ± 0.62 | 10.33 ± 0.71 | 8.56 ± 0.16 | 11.81 ± 1.15 |
| PT-40 | 0.35 ± 0.1 | 0.15 ± 0.04 | 0.16 ± 0.04 | 0.4 ± 0.09 | 0.82 ± 0.13 | 0.48 ± 0.16 | 0.57 ± 0.03 |
| PT-41 | 0.11 ± 0.02 | 0.53 ± 0.13 | 3.03 ± 0.22 | 4.58 ± 0.41 | 5.1 ± 0.18 | 4.5 ± 0.32 | 5.07 ± 0.47 |
| PT-42 | 0.12 ± 0.05 | 1.01 ± 0.04 | 2.17 ± 0.21 | 2.09 ± 0.11 | 4.86 ± 0.17 | 3.16 ± 0.17 | 3.59 ± 0.44 |
| PT-43 | 0.06 ± 0.04 | 1.37 ± 0.11 | 3.63 ± 0.4 | 3.21 ± 0.2 | 5.86 ± 0.41 | 4.46 ± 0.12 | 4.17 ± 0.39 |
| PT-44 | 2.17 ± 0.18 | 4.08 ± 0.81 | 4.23 ± 0.22 | 4.92 ± 0.53 | 7.22 ± 0.36 | 6.57 ± 0.47 | 7.39 ± 1.18 |
| PT-45 | 0.13 ± 0.05 | 0.09 ± 0.02 | 0.1 ± 0.01 | 0.07 ± 0.03 | 0.07 ± 0.02 | 0.12 ± 0.01 | 0.07 ± 0.04 |
| PT-46 | 1.58 ± 0.21 | 1.48 ± 0.22 | 2.79 ± 0.66 | 4.79 ± 0.4 | 4.15 ± 0.47 | 4.23 ± 0.44 | 4.4 ± 0.6 |
| PT-47 | 0.17 ± 0.05 | 1.33 ± 0.44 | 3.67 ± 0.09 | 3.92 ± 0.34 | 5.47 ± 0.49 | 4.05 ± 0.18 | 4.38 ± 0.36 |
| PT-48 | 0.25 ± 0.04 | 0.79 ± 0.12 | 0.67 ± 0.03 | 0.62 ± 0.21 | 2.05 ± 0.33 | 0.71 ± 0.11 | 1.47 ± 0.19 |
| Median | 0.35 | 1.43 | 2.76 | 4.31 | 5.45 | 4.53 | 6.28 |
| activity | | | | | | | |

TABLE 24
Point mutations of IscB with enhanced editing efficiency in human cells.

| | | | | | | | | Target-1 | Target-2 |
|---|---|---|---|---|---|---|---|---|---|
| | | | | | | | | Average | Average |
| | | WT | Mut | Target- | Target- | Target- | Target- | relative | relative |
| Variant | Position | AA | AA | 1 Rep1 | 1 Rep2 | 2 Rep1 | 2 Rep2 | activity | activity |
| C18R | 18 | C | R | 1.03 | 0.96 | 1.32 | 1.04 | 1 ± 0.05 | 1.18 ± 0.2 |
| A34R | 34 | A | R | 1.42 | 1.42 | 1.35 | 1.09 | 1.42 ± 0 | 1.22 ± 0.19 |
| D36R | 36 | D | R | 1.47 | 1.49 | 1.32 | 1.13 | 1.48 ± 0.02 | 1.23 ± 0.13 |
| T37R | 37 | T | R | 1.41 | 1.33 | 1.48 | 1.08 | 1.37 ± 0.06 | 1.28 ± 0.28 |
| Q60I | 60 | O | I | 1.17 | 1.25 | 0.89 | 0.85 | 1.21 ± 0.06 | 0.87 ± 0.03 |
| N66H | 66 | N | H | 1.18 | 1.23 | 1.01 | 1.10 | 1.21 ± 0.04 | 1.06 ± 0.06 |
| I85R | 85 | I | R | 0.93 | 1.11 | 1.15 | 1.10 | 1.02 ± 0.13 | 1.13 ± 0.04 |
| I85K | 85 | I | K | 1.03 | 1.31 | 1.34 | 1.44 | 1.17 ± 0.2 | 1.39 ± 0.07 |
| D97R | 97 | D | R | 1.38 | 1.58 | 1.44 | 1.23 | 1.48 ± 0.14 | 1.34 ± 0.15 |
| D97K | 97 | D | K | 1.40 | 1.72 | 1.56 | 1.54 | 1.56 ± 0.23 | 1.55 ± 0.01 |
| M99R | 99 | M | R | 1.36 | 1.48 | 1.49 | 1.46 | 1.42 ± 0.08 | 1.48 ± 0.02 |
| M99K | 99 | M | K | 1.32 | 1.92 | 1.77 | 1.83 | 1.62 ± 0.42 | 1.8 ± 0.04 |
| A101R | 101 | A | R | 1.17 | 1.49 | 1.19 | 1.28 | 1.33 ± 0.23 | 1.24 ± 0.06 |
| A101K | 101 | A | K | 1.02 | 1.23 | 1.24 | 1.16 | 1.13 ± 0.15 | 1.2 ± 0.06 |
| A104R | 104 | A | R | 1.28 | 1.51 | 1.23 | 1.34 | 1.4 ± 0.16 | 1.29 ± 0.08 |
| E109R | 109 | E | R | 0.98 | 1.10 | 1.17 | 1.30 | 1.04 ± 0.08 | 1.24 ± 0.09 |
| E109K | 109 | E | K | 0.77 | 0.87 | 1.12 | 1.13 | 0.82 ± 0.07 | 1.13 ± 0.01 |
| A112K | 112 | A | K | 0.86 | 1.10 | 1.20 | 1.14 | 0.98 ± 0.17 | 1.17 ± 0.04 |
| R118K | 118 | R | K | 0.88 | 1.00 | 1.08 | 1.14 | 0.94 ± 0.08 | 1.11 ± 0.04 |
| S136R | 136 | S | R | 1.35 | 1.04 | 1.17 | 0.97 | 1.2 ± 0.22 | 1.07 ± 0.14 |
| S136K | 136 | S | K | 1.44 | 1.33 | 1.30 | 1.05 | 1.39 ± 0.08 | 1.18 ± 0.18 |
| E137R | 137 | E | R | 1.10 | 1.12 | 1.08 | 1.02 | 1.11 ± 0.01 | 1.05 ± 0.04 |
| I155R | 155 | I | R | 1.01 | 0.85 | 1.61 | 1.51 | 0.93 ± 0.11 | 1.56 ± 0.08 |
| I155K | 155 | I | K | 0.80 | 0.77 | 1.36 | 1.22 | 0.79 ± 0.02 | 1.29 ± 0.1 |
| L156R | 156 | L | R | 1.29 | 1.35 | 1.46 | 1.28 | 1.32 ± 0.05 | 1.37 ± 0.12 |
| L156K | 156 | L | K | 0.94 | 1.09 | 1.21 | 1.08 | 1.01 ± 0.11 | 1.14 ± 0.09 |
| T157R | 157 | T | R | 1.40 | 1.28 | 1.04 | 0.95 | 1.34 ± 0.09 | 0.99 ± 0.07 |
| T157K | 157 | T | K | 1.17 | 1.30 | 1.11 | 1.02 | 1.24 ± 0.09 | 1.07 ± 0.06 |
| T166R | 166 | T | R | 0.35 | 0.50 | 1.15 | 1.18 | 0.42 ± 0.1 | 1.17 ± 0.02 |
| T166K | 166 | T | K | 0.68 | 0.72 | 1.57 | 1.46 | 0.7 ± 0.03 | 1.52 ± 0.08 |
| L168K | 168 | L | K | 1.35 | 1.20 | 0.63 | 0.74 | 1.27 ± 0.1 | 0.68 ± 0.07 |
| L190K | 190 | L | K | 1.09 | 1.12 | 0.90 | 0.93 | 1.11 ± 0.02 | 0.91 ± 0.02 |
| G204R | 204 | G | R | 1.30 | 1.42 | 0.93 | 0.93 | 1.36 ± 0.08 | 0.93 ± 0 |
| G204K | 204 | G | K | 1.41 | 1.65 | 1.10 | 1.09 | 1.53 ± 0.17 | 1.09 ± 0 |
| N205R | 205 | N | R | 1.14 | 1.48 | 0.89 | 0.86 | 1.31 ± 0.24 | 0.87 ± 0.02 |
| N205K | 205 | N | K | 1.15 | 1.44 | 1.00 | 0.89 | 1.29 ± 0.21 | 0.94 ± 0.07 |
| I206K | 206 | I | K | 1.06 | 1.30 | 0.97 | 0.89 | 1.18 ± 0.17 | 0.93 ± 0.06 |
| K208R | 208 | K | R | 1.08 | 1.15 | 0.88 | 0.97 | 1.11 ± 0.05 | 0.93 ± 0.06 |
| K212R | 212 | K | R | 0.99 | 1.26 | 0.95 | 0.94 | 1.12 ± 0.19 | 0.95 ± 0.01 |
| K214R | 214 | K | R | 1.06 | 1.34 | 1.00 | 0.93 | 1.2 ± 0.2 | 0.96 ± 0.05 |
| N216R | 216 | N | R | 0.95 | 1.33 | 1.21 | 1.11 | 1.14 ± 0.27 | 1.16 ± 0.07 |
| K218R | 218 | K | R | 1.12 | 1.17 | 0.93 | 0.97 | 1.14 ± 0.03 | 0.95 ± 0.03 |
| D219R | 219 | D | R | 0.97 | 1.23 | 1.19 | 1.03 | 1.1 ± 0.18 | 1.11 ± 0.12 |
| D219K | 219 | D | K | 1.05 | 1.28 | 1.31 | 1.17 | 1.16 ± 0.16 | 1.24 ± 0.1 |
| A229R | 229 | A | R | 1.31 | 1.53 | 1.09 | 0.98 | 1.42 ± 0.15 | 1.03 ± 0.08 |
| A229K | 229 | A | K | 1.20 | 1.42 | 0.93 | 0.87 | 1.31 ± 0.15 | 0.9 ± 0.04 |
| K230R | 230 | K | R | 1.12 | 1.26 | 1.02 | 0.97 | 1.19 ± 0.1 | 0.99 ± 0.04 |
| L233R | 233 | L | R | 1.25 | 1.64 | 0.80 | 0.79 | 1.44 ± 0.28 | 0.8 ± 0 |
| L233K | 233 | L | K | 1.49 | 1.97 | 0.82 | 0.71 | 1.73 ± 0.34 | 0.76 ± 0.08 |
| P234R | 234 | P | R | 1.61 | 1.93 | 1.58 | 1.48 | 1.77 ± 0.23 | 1.53 ± 0.08 |
| P234K | 234 | P | K | 1.71 | 2.29 | 1.59 | 1.51 | 2 ± 0.41 | 1.55 ± 0.05 |
| P238R | 238 | P | R | 2.35 | 2.90 | 1.26 | 1.20 | 2.62 ± 0.39 | 1.23 ± 0.04 |
| P238K | 238 | P | K | 2.66 | 3.70 | 1.40 | 1.18 | 3.18 ± 0.74 | 1.29 ± 0.15 |
| F239R | 239 | F | R | 1.41 | 1.67 | 1.20 | 1.02 | 1.54 ± 0.19 | 1.11 ± 0.12 |
| F239K | 239 | F | K | 1.24 | 1.50 | 1.13 | 0.99 | 1.37 ± 0.18 | 1.06 ± 0.1 |
| K241R | 241 | K | R | 1.07 | 1.26 | 1.12 | 1.04 | 1.17 ± 0.14 | 1.08 ± 0.06 |
| E242R | 242 | E | R | 1.26 | 1.51 | 1.05 | 0.89 | 1.39 ± 0.17 | 0.97 ± 0.11 |
| E242K | 242 | E | K | 1.26 | 1.61 | 1.02 | 1.04 | 1.43 ± 0.25 | 1.03 ± 0.02 |
| A245R | 245 | A | R | 1.39 | 1.73 | 0.92 | 0.92 | 1.56 ± 0.24 | 0.92 ± 0 |
| A245K | 245 | A | K | 1.37 | 1.67 | 1.03 | 1.08 | 1.52 ± 0.21 | 1.06 ± 0.04 |
| E246R | 246 | E | R | 1.20 | 1.82 | 1.05 | 1.03 | 1.51 ± 0.44 | 1.04 ± 0.01 |
| E246K | 246 | E | K | 1.28 | 1.56 | 1.07 | 1.02 | 1.42 ± 0.2 | 1.05 ± 0.04 |
| E248R | 248 | E | R | 1.05 | 1.33 | 0.95 | 0.91 | 1.19 ± 0.2 | 0.93 ± 0.03 |
| T252R | 252 | T | R | 1.67 | 1.76 | 0.97 | 1.05 | 1.72 ± 0.06 | 1.01 ± 0.05 |
| T252K | 252 | T | K | 1.36 | 1.61 | 1.01 | 1.11 | 1.49 ± 0.18 | 1.06 ± 0.07 |
| E254R | 254 | E | R | 2.57 | 2.85 | 1.49 | 1.38 | 2.71 ± 0.2 | 1.44 ± 0.08 |
| E254K | 254 | E | K | 2.49 | 2.69 | 1.45 | 1.24 | 2.59 ± 0.15 | 1.34 ± 0.15 |
| K255R | 255 | K | R | 1.18 | 1.18 | 1.11 | 0.97 | 1.18 ± 0 | 1.04 ± 0.1 |
| S256R | 256 | S | R | 1.46 | 1.51 | 1.38 | 1.10 | 1.48 ± 0.03 | 1.24 ± 0.2 |
| S256K | 256 | S | K | 1.51 | 1.54 | 1.29 | 1.06 | 1.52 ± 0.02 | 1.17 ± 0.16 |
| T257R | 257 | T | R | 1.45 | 1.18 | 1.13 | 1.02 | 1.32 ± 0.19 | 1.08 ± 0.08 |
| T257K | 257 | T | K | 1.29 | 1.06 | 1.02 | 0.85 | 1.17 ± 0.16 | 0.93 ± 0.12 |
| F258R | 258 | F | R | 1.62 | 1.64 | 1.34 | 1.23 | 1.63 ± 0.01 | 1.28 ± 0.08 |
| F258K | 258 | F | K | 1.48 | 1.58 | 1.38 | 1.12 | 1.53 ± 0.07 | 1.25 ± 0.18 |
| S279R | 279 | S | R | 1.23 | 1.08 | 0.98 | 0.99 | 1.16 ± 0.11 | 0.99 ± 0.01 |
| G280R | 280 | G | R | 1.20 | 1.04 | 0.92 | 0.88 | 1.12 ± 0.11 | 0.9 ± 0.03 |
| L291R | 291 | L | R | 1.37 | 1.47 | 1.21 | 1.13 | 1.42 ± 0.07 | 1.17 ± 0.06 |
| L291K | 291 | L | K | 1.26 | 1.20 | 1.15 | 1.07 | 1.23 ± 0.04 | 1.11 ± 0.05 |
| K293R | 293 | K | R | 1.09 | 0.95 | 1.27 | 1.13 | 1.02 ± 0.09 | 1.2 ± 0.1 |
| Q298K | 298 | Q | K | 0.86 | 0.89 | 1.21 | 1.09 | 0.88 ± 0.02 | 1.15 ± 0.09 |
| E303R | 303 | E | R | 1.20 | 1.06 | 1.28 | 1.13 | 1.13 ± 0.1 | 1.21 ± 0.1 |
| E303K | 303 | E | K | 1.31 | 1.41 | 1.23 | 1.26 | 1.36 ± 0.07 | 1.24 ± 0.02 |
| M304K | 304 | M | K | 1.39 | 1.22 | 0.96 | 0.99 | 1.3 ± 0.12 | 0.98 ± 0.02 |
| K306R | 306 | K | R | 1.03 | 0.93 | 1.22 | 1.15 | 0.98 ± 0.07 | 1.19 ± 0.05 |
| R307K | 307 | R | K | 1.08 | 1.06 | 1.20 | 1.02 | 1.07 ± 0.01 | 1.11 ± 0.12 |
| C312R | 312 | C | R | 1.02 | 0.94 | 1.19 | 1.13 | 0.98 ± 0.05 | 1.16 ± 0.05 |
| C312K | 312 | C | K | 0.76 | 0.68 | 1.15 | 1.08 | 0.72 ± 0.06 | 1.12 ± 0.04 |
| T319K | 319 | T | K | 1.42 | 1.33 | 1.19 | 1.16 | 1.37 ± 0.06 | 1.17 ± 0.02 |
| E323R | 323 | E | R | 2.63 | 2.41 | 1.50 | 1.47 | 2.52 ± 0.16 | 1.48 ± 0.02 |
| E323K | 323 | E | K | 2.69 | 2.77 | 1.54 | 1.43 | 2.73 ± 0.05 | 1.49 ± 0.08 |
| E328R | 328 | E | R | 1.65 | 1.37 | 1.02 | 1.19 | 1.51 ± 0.2 | 1.11 ± 0.12 |
| Q329R | 329 | Q | R | 1.13 | 1.04 | 1.13 | 1.10 | 1.08 ± 0.06 | 1.12 ± 0.02 |
| D331R | 331 | D | R | 1.28 | 1.28 | 1.22 | 1.22 | 1.28 ± 0 | 1.22 ± 0 |
| G332R | 332 | G | R | 0.70 | 0.65 | 1.15 | 1.13 | 0.68 ± 0.04 | 1.14 ± 0.01 |
| G338R | 338 | G | R | 1.40 | 1.10 | 1.13 | 1.11 | 1.25 ± 0.21 | 1.12 ± 0.01 |
| S341R | 341 | S | R | 1.21 | 1.06 | 0.94 | 0.94 | 1.13 ± 0.11 | 0.94 ± 0 |
| R353K | 353 | R | K | 1.13 | 1.26 | 1.12 | 1.07 | 1.2 ± 0.09 | 1.1 ± 0.04 |
| A360R | 360 | A | R | 1.51 | 1.34 | 1.21 | 1.08 | 1.43 ± 0.12 | 1.15 ± 0.09 |
| A360K | 360 | A | K | 1.51 | 1.44 | 1.17 | 1.15 | 1.48 ± 0.05 | 1.16 ± 0.01 |
| K368R | 368 | K | R | 1.21 | 1.21 | 1.04 | 0.91 | 1.21 ± 0 | 0.98 ± 0.09 |
| L372R | 372 | L | R | 1.43 | 1.47 | 1.24 | 1.04 | 1.45 ± 0.03 | 1.14 ± 0.14 |
| D376R | 376 | D | R | 1.17 | 1.38 | 1.20 | 1.08 | 1.28 ± 0.15 | 1.14 ± 0.08 |
| A377R | 377 | A | R | 1.36 | 1.66 | 1.22 | 1.11 | 1.51 ± 0.21 | 1.17 ± 0.08 |
| D378R | 378 | D | R | 1.35 | 1.56 | 1.19 | 1.07 | 1.46 ± 0.15 | 1.13 ± 0.08 |
| T379R | 379 | T | R | 1.15 | 1.22 | 1.18 | 1.12 | 1.19 ± 0.05 | 1.15 ± 0.04 |
| A380R | 380 | A | R | 1.11 | 1.12 | 1.19 | 1.07 | 1.12 ± 0.01 | 1.13 ± 0.08 |
| E381R | 381 | E | R | 1.78 | 1.84 | 1.30 | 1.25 | 1.81 ± 0.04 | 1.28 ± 0.04 |
| S382R | 382 | S | R | 1.31 | 1.21 | 1.04 | 1.02 | 1.26 ± 0.07 | 1.03 ± 0.01 |
| T384R | 384 | T | R | 1.22 | 1.51 | 1.31 | 1.21 | 1.37 ± 0.21 | 1.26 ± 0.07 |
| E385R | 385 | E | R | 1.76 | 2.03 | 1.45 | 1.18 | 1.9 ± 0.19 | 1.32 ± 0.19 |
| M386R | 386 | M | R | 1.43 | 1.42 | 1.18 | 1.06 | 1.43 ± 0.01 | 1.12 ± 0.08 |
| T388R | 388 | T | R | 1.36 | 1.50 | 1.34 | 1.12 | 1.43 ± 0.1 | 1.23 ± 0.16 |
| L390R | 390 | L | R | 1.79 | 1.60 | 1.26 | 1.20 | 1.7 ± 0.13 | 1.23 ± 0.04 |
| L390K | 390 | L | K | 1.97 | 1.97 | 1.41 | 1.43 | 1.97 ± 0 | 1.42 ± 0.01 |
| M391R | 391 | M | R | 2.22 | 2.51 | 1.52 | 1.61 | 2.37 ± 0.21 | 1.57 ± 0.06 |
| M391K | 391 | M | K | 2.34 | 2.47 | 1.50 | 1.47 | 2.41 ± 0.09 | 1.49 ± 0.02 |
| K393R | 393 | K | R | 1.19 | 1.31 | 1.15 | 0.98 | 1.25 ± 0.08 | 1.07 ± 0.12 |
| P405K | 405 | P | K | 0.99 | 1.39 | 0.90 | 1.13 | 1.19 ± 0.28 | 1.01 ± 0.16 |
| K406R | 406 | K | R | 0.95 | 1.31 | 1.09 | 1.14 | 1.13 ± 0.25 | 1.12 ± 0.04 |
| V408R | 408 | V | R | 0.97 | 1.39 | 1.02 | 1.19 | 1.18 ± 0.3 | 1.1 ± 0.12 |
| E409K | 409 | E | K | 1.89 | 2.06 | 1.18 | 1.26 | 1.97 ± 0.12 | 1.22 ± 0.06 |
| E409R | 409 | E | R | 1.83 | 2.28 | 1.20 | 1.31 | 2.05 ± 0.32 | 1.26 ± 0.08 |
| T410R | 410 | T | R | 1.12 | 1.41 | 1.19 | 1.37 | 1.27 ± 0.21 | 1.28 ± 0.13 |
| A412R | 412 | A | R | 1.17 | 1.60 | 0.99 | 1.31 | 1.39 ± 0.31 | 1.15 ± 0.23 |
| D413R | 413 | D | R | 1.19 | 1.66 | 1.00 | 1.06 | 1.43 ± 0.33 | 1.03 ± 0.05 |
| P416R | 416 | P | R | 1.09 | 1.17 | 1.08 | 1.35 | 1.13 ± 0.05 | 1.21 ± 0.19 |
| G417R | 417 | G | R | 0.94 | 1.08 | 1.05 | 1.16 | 1.01 ± 0.1 | 1.1 ± 0.08 |
| H418K | 418 | H | K | 1.02 | 1.20 | 0.96 | 1.11 | 1.11 ± 0.13 | 1.03 ± 0.11 |
| F419R | 419 | F | R | 1.07 | 1.33 | 0.80 | 0.97 | 1.2 ± 0.18 | 0.88 ± 0.13 |
| V421R | 421 | V | R | 0.99 | 1.21 | 0.95 | 1.17 | 1.1 ± 0.15 | 1.06 ± 0.15 |
| V421K | 421 | V | K | 1.18 | 1.58 | 1.07 | 1.12 | 1.38 ± 0.29 | 1.1 ± 0.03 |
| N423R | 423 | N | R | 1.29 | 1.51 | 0.73 | 0.87 | 1.4 ± 0.16 | 0.8 ± 0.1 |
| W425K | 425 | W | K | 0.24 | 0.30 | 1.06 | 1.27 | 0.27 ± 0.04 | 1.16 ± 0.15 |
| E437R | 437 | E | R | 1.27 | 1.71 | 1.06 | 1.09 | 1.49 ± 0.31 | 1.08 ± 0.02 |
| D439R | 439 | D | R | 1.72 | 2.09 | 1.16 | 1.43 | 1.91 ± 0.26 | 1.3 ± 0.19 |
| D439K | 439 | D | K | 1.57 | 1.91 | 1.09 | 1.23 | 1.74 ± 0.24 | 1.16 ± 0.1 |
| E455R | 455 | E | R | 0.94 | 1.23 | 1.05 | 1.30 | 1.09 ± 0.2 | 1.18 ± 0.18 |
| E462R | 462 | E | R | 1.06 | 1.28 | 0.99 | 1.14 | 1.17 ± 0.16 | 1.07 ± 0.11 |
| P463R | 463 | P | R | 1.34 | 1.90 | 0.94 | 1.17 | 1.62 ± 0.4 | 1.05 ± 0.16 |

The effect of point mutations was evaluated by measuring the editing efficiency in HEK293-FT cells over two genomic targets. The editing efficiency of each mutation was normalized to the Wild-type nuclease, and reported as the relative activity in Table 23. Point mutations enhanced activity for 10% or above (i.e. Relative activity > 1.1) in either or both targets were reported.

### Methods:

To evaluate the effect of IscB point mutations, the corresponding nuclease- and guide-expression plasmids were co-transfected into HEK293-FT cells using LIPOFECTAMINE 2000 with technical triplicates. Cells were harvested 48 hours post-transfection, and the genomic DNA was extracted by QUICKEXTRACT solution. Genomic region containing the intended target site of IscB was amplified with target-specific primer pairs (Tables 24-25) using PHUSION U DNA polymerase (THERMO), and barcoded with standard ILLUMINA adapter primers in the 2^{nd} round PCR for multiplexed sequencing on a NEXTSEQ 2000 instrument (ILLUMINA). Sequencing data was processed and analyzed by in-house pipeline to measure the nuclease-induced, guide RNA dependent indels (i.e. editing efficiency). The editing efficiency of technical triplicates for a given nuclease variant were averaged, and normalized to the WT nuclease, as the relative activity reported in Table 23. Variants enhanced the activity for 10% or above in either or both targets are listed in Table 23.

The genomic targets of IscB and corresponding primers for amplification.

| | | Target-specific primer | Target-specific primer |
|---|---|---|---|
| | Target sequence (5′-3′) | (Forward, 5′-3′) | (Reverse, 5′-3′) |
| Target-1 | TGAACTCATATCTGTAAGAA | CACTATGAGCCTATAG | GAAAATTGACTGCTTC |
| | (SEQ ID NO: 182) | ACTATCAGTT | TTACTTTTCTAA |
| | | (SEQ ID NO: 215) | (SEQ ID NO: 216) |
| Target-2 | GCCATGACAACTGGGTGGAA | CTCACCTCTCCAAAGCTG | GAAAGGCCCATTGCTCT |
| | (SEQ ID NO: 217) | (SEQ ID NO: 218) | (SEQ ID NO: 219) |

Table 25 (above). The 20-bp genomic sequences targeted by RNA-guided IscB nuclease, and corresponding PCR primers are listed. Of note, the standard ILLUMINA partial adapter sequences appended on the 5′ of each primer were not shown.

### Example 4: IscB OmegaRNA Trimming

EboIscB is a RNA-guided endonuclease with measurable activity in human cells. Prior work by Altae-Tan et al., defined the RNA component of EboIscB (i.e. OmegaRNA) as a single sequence with 14-25 nt variable region at the 5′-end which guides the endonuclease to the target site by RNA-DNA complementarity, followed by a 205 nt conserved region. To improve the utility of EboIscB as gene editing tool in human cells, we determined the optimal length of the variable region, and performed a structural-guided engineering of the conserved region to miniaturize the length of OmegaRNA. The OmegaRNA was truncated to 146~153-nt with activity on-par or even higher than the wild-type sequence (225-nt). These truncated OmegaRNA can be chemical synthesized, and laid foundation for further engineering of the RNA component of EboIscB.

### Results

To define the optimal length of the variable region, the on-target activity of EboIscB was first measured in human cells using WT OmegaRNA with 12-20 nt variable region over 4 target sites. Nuclease and guide expression plasmids were co-transfected into HEK293-FT cells using LIPOFECTAMINE 2000 under recommended condition. Gene editing activity of EboIscB was measured by amplicon sequencing 2-days post transfection. While previous study by Altae-Tan et al., showed indistinguishable activity of EboIscB with 16~20 nt variable region in vitro, the result demonstrated that the 20-nt variable region outperformed other lengths in human cells (Tables 1-4). The variable region was further extended up to 25-nt, and again, the highest activity of pEDIT152 (EboIscB-REC-Triple) was observed with 20-nt variable region (Tables 5-6). These results defined the 20-nt variable region as the optimal length for EboIscB-mediated human cell editing.

The conserved region of OmegaRNA was engineered next. With a 20-nt variable region, WT EboIscB OmegaRNA (225-nt) is out of the scope of chemical RNA synthesis. To enable the gene editing of EboIscB with synthetic components, redundant structural elements were removed within the OmegaRNA. The folding of OmegaRNA was predicted with MXfold2 (dna.bio.keio.ac.jp/mxfold2/) and defined the structural element according to the nomenclature described in Schuler et al., “Structural basis for RNA-guided DNA cleavage by IscB-ωRNA and mechanistic comparison with Cas9,” Science 2022 Jun. 24, 376 (6600): 1476-1481, the contents of which are incorporated herein by reference in their entirety (FIG. 4A). Based on the predicted structure, the length of P1 loop was reduced (position 2-67) by gradually removing base-pairs from the top of the predicted stem-loop. A GAAAA or GAAA linker was added to seal the trimmed stem (Table 7). The in-cell data revealed that position 12-57 of the P1 loop can be removed without affecting the editing efficiency of pEDIT152 (EboIscB-REC-Triple) over the VEGFA-3 target (Table 7), which was further validated over another 3 target sites (Table 8-10).

A similar strategy was next applied to trim the predicted P5 loop, and revealed the removal of position 156-175 of OmegaRNA is well-tolerated without affecting editing efficiency (Table 11). Lastly, the 3′ of the OmegaRNA was trimmed, identifying that up to 20-nt can be removed without abolishing the activity in cells (Table 12). Combining these strategies together enabled gene editing of pEDIT152 (EboIscB-REC-Triple) with 146-153 nt OmegaRNA (including a 20-nt variable region) without compromising editing efficiency in human cells (Table 13). Of note, the M71 version of OmegaRNA (FIG. 4C, 153-nt) maintained or enhanced editing efficiency of pEDIT152 (EboIscB-REC-Triple) over all target sites (Table 13), indicating the RNA engineering can be an alternative route to improve nuclease activity beyond protein mutagenesis. The performance of M71 OmegaRNA was further evaluated in combination of EboIscB-REC nucleases with no (pEDIT71) or 7 point mutations (pEDIT420), and observed the same positive effect on activity (Table 14-15), suggesting the performance of truncated OmegaRNA is independent of the point mutations introduced on the EboIscB construct.

Given these observations, the OmegaRNA was further engineered to improve its functionality using M71 as the new scaffold. The OmegaRNA was further trimmed, where increasing the folding strength of various structure elements, and extending the basepairing region of the predicted pseudoknot, can lead to higher editing efficiency of EboIscB-REC (Table 19).

Tables 1-6. Gene editing activity of EboIscB in human HEK293-FT cells with 12-20 nt or 16-25 nt variable sequences in the OmegaRNA. See Table 16 for the exact sequences of variable region of the OmegaRNA. EboIscB-REC with 3 additional point mutations (pEDIT152) was used in this experiment.

TABLE 1
Target 1 (VEGFA-3) Editing efficiency (%)

| Length of | | | | | Relative |
|---|---|---|---|---|---|
| variable | Repli- | Repli- | Repli- | | activity |
| region (nt) | cate-1 | cate-2 | cate-3 | Average | to 20- nt |
| 12 | 0.1 | 0.11 | 0.14 | 0.12 ± 0.02 | 0.00 |
| 13 | 0.47 | 0.38 | 0.54 | 0.46 ± 0.08 | 0.01 |
| 14 | 4.71 | 3.87 | 4.23 | 4.27 ± 0.42 | 0.14 |
| 15/16 | 17.57 | 13.3 | 16.6 | 15.82 ± 2.24 | 0.50 |
| 17 | 14.37 | 13.18 | 12.17 | 13.24 ± 1.1 | 0.42 |
| 18/19 | 16.54 | 14.97 | 14.19 | 15.23 ± 1.2 | 0.48 |
| 20 | 34.19 | 28.71 | 31.96 | 31.62 ± 2.76 | 1.00 |

TABLE 2
Target 2 (AAVS1-2) Editing efficiency (%)

| Length of | | | | | Relative |
|---|---|---|---|---|---|
| variable | Repli- | Repli- | Repli- | | activity |
| region (nt) | cate-1 | cate-2 | cate-3 | Average | to 20- nt |
| 12 | 0.01 | 0.01 | 0.01 | 0.01 ± 0 | 0.00 |
| 13 | 0.05 | 0.02 | 0.04 | 0.04 ± 0.02 | 0.00 |
| 14/15 | 3.97 | 3.27 | 3.61 | 3.62 ± 0.35 | 0.28 |
| 16 | 5.45 | 4.83 | 5.45 | 5.24 ± 0.36 | 0.41 |
| 17 | 1.41 | 0.99 | 1.12 | 1.17 ± 0.22 | 0.09 |
| 18 | 4.98 | 3.74 | 5.28 | 4.67 ± 0.82 | 0.37 |
| 19/20 | 13.24 | 11.31 | 13.64 | 12.73 ± 1.25 | 1.00 |

TABLE 3
Target 3 (HPRT-7) Editing efficiency (%)

| Length of | | | | | Relative |
|---|---|---|---|---|---|
| variable | Repli- | Repli- | Repli- | | activity |
| region (nt) | cate-1 | cate-2 | cate-3 | Average | to 20- nt |
| 12 | 0 | 0.02 | 0.01 | 0.01 ± 0.01 | 0.00 |
| 13 | 0.01 | 0.01 | 0 | 0.01 ± 0.01 | 0.00 |
| 14 | 0.7 | 0.75 | 0.66 | 0.7 ± 0.05 | 0.12 |
| 15 | 2.94 | 3.12 | 3.21 | 3.09 ± 0.14 | 0.52 |
| 16 | 2 | 1.7 | 2.15 | 1.95 ± 0.23 | 0.33 |
| 17 | 3.62 | 3.04 | 3.43 | 3.36 ± 0.3 | 0.57 |
| 18/19 | 4.01 | 3.35 | 4.11 | 3.82 ± 0.41 | 0.64 |
| 20 | 5.72 | 5.71 | 6.39 | 5.94 ± 0.39 | 1.00 |

TABLE 4
Target 4 (HPRT-12) Editing efficiency (%)

| Length of | | | | | Relative |
|---|---|---|---|---|---|
| variable | Repli- | Repli- | Repli- | | activity |
| region (nt) | cate-1 | cate-2 | cate-3 | Average | to 20- nt |
| 12 | 0.03 | 0.02 | 0.02 | 0.02 ± 0.01 | 0.00 |
| 13 | 0.02 | 0.01 | 0.03 | 0.02 ± 0.01 | 0.00 |
| 14 | 1.18 | 1.11 | 1.54 | 1.28 ± 0.23 | 0.19 |
| 15 | 5.63 | 4.92 | 5.18 | 5.24 ± 0.36 | 0.79 |
| 16 | 4.6 | 4.59 | 4.45 | 4.55 ± 0.08 | 0.68 |
| 17 | 3.39 | 3.95 | 3.46 | 3.6 ± 0.31 | 0.54 |
| 18/19 | 5.87 | 5.36 | 6.37 | 5.87 ± 0.51 | 0.88 |
| 20 | 6.86 | 6.33 | 6.73 | 6.64 ± 0.28 | 1.00 |

TABLE 5
Target 1 (VEGFA-3) Editing efficiency (%)

| Length of | | | | | Relative |
|---|---|---|---|---|---|
| variable | Repli- | Repli- | Repli- | | activity |
| region (nt) | cate-1 | cate-2 | cate-3 | Average | to 20- nt |
| 16 | 9.95 | 8.94 | 8.01 | 8.97 ± 0.97 | 0.49 |
| 17 | 7.92 | 8.03 | 7.92 | 7.96 ± 0.06 | 0.43 |
| 18/19 | 11.1 | 11.37 | 9.89 | 10.79 ± 0.79 | 0.59 |
| 20 | 18.49 | 18.83 | 17.7 | 18.34 ± 0.58 | 1.00 |
| 21 | 7.52 | 6.51 | 5.18 | 6.4 ± 1.17 | 0.35 |
| 22 | 10.61 | 8.35 | 7.07 | 8.68 ± 1.79 | 0.47 |
| 23/24 | 5.94 | 6.74 | 5.37 | 6.02 ± 0.69 | 0.33 |
| 25 | 8.22 | 6.7 | 6.93 | 7.28 ± 0.82 | 0.40 |

TABLE 6
Target 2 (AAVS1-2) Editing efficiency (%)

| Length of | | | | | Relative |
|---|---|---|---|---|---|
| variable | Repli- | Repli- | Repli- | | activity |
| region (nt) | cate-1 | cate-2 | cate-3 | Average | to 20- nt |
| 16 | 3.07 | 5.36 | 4.35 | 4.26 ± 1.15 | 0.65 |
| 17 | 0.89 | 0.78 | 0.81 | 0.83 ± 0.06 | 0.13 |
| 18 | 2.9 | 2.91 | 3.08 | 2.96 ± 0.1 | 0.45 |
| 19/20 | 6.59 | 6.04 | 7.01 | 6.55 ± 0.49 | 1.00 |
| 21 | 2.81 | 2.41 | 2.94 | 2.72 ± 0.28 | 0.42 |
| 22 | 1.62 | 1.54 | 1.7 | 1.62 ± 0.08 | 0.25 |
| 23 | 0.82 | 0.71 | 0.64 | 0.72 ± 0.09 | 0.11 |
| 24 | 0.83 | 0.7 | 0.69 | 0.74 ± 0.08 | 0.11 |
| 25 | 0.61 | 0.68 | 0.61 | 0.63 ± 0.04 | 0.10 |

TABLE 7
Editing efficiency of EboIscB at VEGFA-3 target site in human cells with
rationally engineered OmegaRNA within the P1 loop. See Table 17 for the OmegaRNA
sequence of each construct. A 20-nt sequence targeting VEGFA-3 (Table 16) was
used as the 48variable region of the OmegaRNA. EboIscB-REC with 3 additional point
mutations (pEDIT152) was used in this experiment.

| | Trun- | Trun- | | | | | Average | Relative |
|---|---|---|---|---|---|---|---|---|
| | cation | cation | | Rep1 | Rep2 | Rep3 | activity | activity |
| | start | end | Linker | (%) | (%) | (%) | (%) | to WT |
| VEGFA- | 32 | 77 | GAAAA | 17.66 | 17.07 | 16.9 | 17.21 ± 0.4 | 0.79 |
| 3-M1 | | | | | | | | |
| VEGFA- | 34 | 75 | GAAAA | 21.44 | 19.57 | 17.49 | 19.5 ± 1.98 | 0.89 |
| 3-M2 | | | | | | | | |
| VEGFA- | 36 | 74 | GAAAA | 15.56 | 15.85 | 13.62 | 15.01 ± 1.21 | 0.69 |
| 3-M3 | | | | | | | | |
| VEGFA- | 38 | 72 | GAAAA | 16.56 | 17.08 | 15.63 | 16.42 ± 0.73 | 0.75 |
| 3-M4 | | | | | | | | |
| VEGFA- | 40 | 70 | GAAAA | 11.91 | 11.75 | 10.75 | 11.47 ± 0.63 | 0.53 |
| 3-M5 | | | | | | | | |
| VEGFA- | 42 | 68 | GAAAA | 9.21 | 9 | 8.89 | 9.03 ± 0.16 | 0.41 |
| 3-M6 | | | | | | | | |
| VEGFA- | 44 | 66 | GAAAA | 16.94 | 20.37 | 16.46 | 17.92 ± 2.13 | 0.82 |
| 3-M7 | | | | | | | | |
| VEGFA- | 46 | 65 | GAAAA | 20.99 | 20.2 | 19.54 | 20.24 ± 0.73 | 0.93 |
| 3-M8 | | | | | | | | |
| VEGFA- | 48 | 63 | GAAAA | 5.13 | 5.19 | 5.22 | 5.18 ± 0.05 | 0.24 |
| 3-M9 | | | | | | | | |
| VEGFA- | 50 | 61 | GAAAA | 11.35 | 12.95 | 12.69 | 12.33 ± 0.86 | 0.57 |
| 3-M10 | | | | | | | | |
| VEGFA- | 52 | 59 | GAAAA | 16.05 | 16.65 | 15.21 | 15.97 ± 0.72 | 0.73 |
| 3-M11 | | | | | | | | |
| VEGFA- | 32 | 77 | GAAA | 22.27 | 20.41 | 22.47 | 21.72 ± 1.14 | 1 |
| 3-M25 | | | | | | | | |
| VEGFA- | 34 | 75 | GAAA | 18.48 | 18.9 | 21.28 | 19.55 ± 1.51 | 0.9 |
| 3-M26 | | | | | | | | |
| VEGFA- | 36 | 74 | GAAA | 15.95 | 16.28 | 14.97 | 15.73 ± 0.68 | 0.72 |
| 3-M27 | | | | | | | | |
| VEGFA- | 38 | 72 | GAAA | 16 | 19.02 | 17.22 | 17.41 ± 1.52 | 0.8 |
| 3-M28 | | | | | | | | |
| VEGFA- | 40 | 70 | GAAA | 9.62 | 11.38 | 12.54 | 11.18 ± 1.47 | 0.51 |
| 3-M29 | | | | | | | | |
| VEGFA- | 42 | 68 | GAAA | 8.83 | 11.81 | 11.76 | 10.8 ± 1.71 | 0.5 |
| 3-M30 | | | | | | | | |
| VEGFA- | 44 | 66 | GAAA | 20.48 | 21.73 | 19.89 | 20.7 ± 0.94 | 0.95 |
| 3-M31 | | | | | | | | |
| VEGFA- | 46 | 65 | GAAA | 18.03 | 15.42 | 16.68 | 16.71 ± 1.31 | 0.77 |
| 3-M32 | | | | | | | | |
| VEGFA- | 48 | 63 | GAAA | 13.98 | 14.29 | 13.51 | 13.93 ± 0.39 | 0.64 |
| 3-M33 | | | | | | | | |
| VEGFA- | 50 | 61 | GAAA | 15.77 | 16.67 | 17.67 | 16.7 ± 0.95 | 0.77 |
| 3-M34 | | | | | | | | |
| VEGFA- | 52 | 59 | GAAA | 16.64 | 19.46 | 17.27 | 17.79 ± 1.48 | 0.82 |
| 3-M35 | | | | | | | | |
| VEGFA- | — | — | — | 21.38 | 22.08 | 21.95 | 21.8 ± 0.37 | 1 |
| 3 (WT) | | | | | | | | |

Tables 8-10. Editing efficiency of EboIscB at 3 target sites in human cells with rationally engineered OmegaRNA within the P1 loop. See Table 17 for the OmegaRNA sequence of each construct. 20-nt sequence targeting each genomic locus (Table 16) was used as the variable region of the OmegaRNA. EboIscB-REC with 3 additional point mutations (pEDIT152) was used in this experiment.

TABLE 8

| | Trunc- | Trunc- | | | | | Average | Relative |
|---|---|---|---|---|---|---|---|---|
| | ation | ation | | Rep1 | Rep2 | Rep3 | activity | activity |
| | start | end | Linker | (%) | (%) | (%) | (%) | to WT |
| AAVS1- | 32 | 77 | GAAAA | 6.69 | 8.04 | 8.2 | 7.64 ± 0.83 | 1.22 |
| 2-M25 | | | | | | | | |
| AAVS1- | 34 | 75 | GAAAA | 3.85 | 6.02 | 4.99 | 4.95 ± 1.09 | 0.79 |
| 2-M26 | | | | | | | | |
| AAVS1- | 36 | 74 | GAAAA | 3.87 | 3.17 | 3.43 | 3.49 ± 0.35 | 0.56 |
| 2-M27 | | | | | | | | |
| AAVS1- | 38 | 72 | GAAAA | 4.52 | 4.02 | 4.41 | 4.32 ± 0.26 | 0.69 |
| 2-M28 | | | | | | | | |
| AAVS1- | — | — | — | 5.52 | 6.8 | 6.51 | 6.28 ± 0.67 | 1 |
| 2 (WT) | | | | | | | | |

TABLE 9

| | Trunc- | Trunc- | | | | | Average | Relative |
|---|---|---|---|---|---|---|---|---|
| | ation | ation | | Rep1 | Rep2 | Rep3 | activity | activity |
| | start | end | Linker | (%) | (%) | (%) | (%) | to WT |
| HPRT-7- | 32 | 77 | GAAAA | 3.13 | 3.47 | 3.76 | 3.45 ± 0.32 | 1.12 |
| M25 | | | | | | | | |
| HPRT-7- | 34 | 75 | GAAAA | 2.2 | 2.43 | 2.48 | 2.37 ± 0.15 | 0.77 |
| M26 | | | | | | | | |
| HPRT-7- | 36 | 74 | GAAAA | 1.17 | 1.07 | 1.1 | 1.11 ± 0.05 | 0.36 |
| M27 | | | | | | | | |
| HPRT-7- | 38 | 72 | GAAAA | 1.81 | 1.64 | 1.64 | 1.7 ± 0.1 | 0.55 |
| M28 | | | | | | | | |
| HPRT-7 | — | — | — | 2.55 | 3.31 | 3.36 | 3.07 ± 0.45 | 1 |
| (WT) | | | | | | | | |

TABLE 10

| | Trunc- | Trunc- | | | | | Average | Relative |
|---|---|---|---|---|---|---|---|---|
| | ation | ation | | Rep1 | Rep2 | Rep3 | activity | activity |
| | start | end | Linker | (%) | (%) | (%) | (%) | to WT |
| HPRT- | 32 | 77 | GAAAA | 5.54 | 6.59 | 6.45 | 6.19 ± 0.57 | 1.53 |
| 12-M25 | | | | | | | | |
| HPRT- | 34 | 75 | GAAAA | 3.84 | 3.08 | 3.63 | 3.52 ± 0.39 | 0.87 |
| 12-M26 | | | | | | | | |
| HPRT- | 36 | 74 | GAAAA | 3.04 | 2.86 | 2.89 | 2.93 ± 0.1 | 0.72 |
| 12-M27 | | | | | | | | |
| HPRT- | 38 | 72 | GAAAA | 3.07 | 3.41 | 2.67 | 3.05 ± 0.37 | 0.75 |
| 12-M28 | | | | | | | | |
| HPRT- | — | — | — | 3.3 | 5.4 | 3.48 | 4.06 ± 1.16 | 1 |
| 12 (WT) | | | | | | | | |

TABLE 11
Editing efficiency of EboIscB at VEGFA-3 site in human cells with
engineered OmegaRNA at P5 loop. See Table 17 for the OmegaRNA sequence of each
construct. A 20-nt sequence targeting VEGFA-3 (Table 16) was used as the variable
region of the OmegaRNA. EboIscB-REC with 3 additional point mutations (pEDIT152)
was used in this experiment.

| | Trunc- | Trunc- | | | | | Average | Relative |
|---|---|---|---|---|---|---|---|---|
| | ation | ation | | Rep1 | Rep2 | Rep3 | activity | activity |
| | start | end | Linker | (%) | (%) | (%) | (%) | to WT |
| VEGFA- | 179 | 192 | GAAA | 19.64 | 20.91 | 21.07 | 20.54 ± 0.78 | 0.9 |
| 3-M52 | | | | | | | | |
| VEGFA- | 180 | 191 | GAAA | 0.1 | 0.25 | 0.19 | 0.18 ± 0.08 | 0.0 |
| 3-M53 | | | | | | | | |
| VEGFA- | 181 | 190 | GAAA | 0.18 | 0.2 | 0.18 | 0.19 ± 0.01 | 0.0 |
| 3-M54 | | | | | | | | |
| VEGFA- | 183 | 188 | GAAA | 20.61 | 22.79 | 19.62 | 21.01 ± 1.62 | 1.0 |
| 3-M55 | | | | | | | | |
| VEGFA- | 178 | 193 | GAAA | 20.37 | 22.6 | 21.3 | 21.42 ± 1.12 | 1.0 |
| 3-M56 | | | | | | | | |
| VEGFA- | 176 | 195 | GAAA | 19.32 | 21.7 | 21.76 | 20.93 ± 1.39 | 1.0 |
| 3-M57 | | | | | | | | |
| VEGFA- | 174 | 197 | GAAA | 9.04 | 11.99 | 12.17 | 11.07 ± 1.76 | 0.5 |
| 3-M58 | | | | | | | | |
| VEGFA- | — | — | — | 21.38 | 22.08 | 21.95 | 21.8 ± 0.37 | 1.0 |
| 3 (WT) | | | | | | | | |

TABLE 12
Editing efficiency of EboIscB at VEGFA-3 site in
human cells with truncated OmegaRNA at 3′-end.

| | Trun- | Trun- | | | | Average | Relative |
|---|---|---|---|---|---|---|---|
| | cation | cation | | | | activity | activity to |
| Table 12 | start | end | Rep1 (%) | Rep2 (%) | Rep3 (%) | (%) | WT |
| VEGFA- | 224 | 225 | 20.96 | 21.86 | 20.84 | 21.22 ± 0.56 | 1.0 |
| 3-M40 | | | | | | | |
| VEGFA- | 222 | 225 | 19.26 | 19.65 | 20.81 | 19.91 ± 0.81 | 0.9 |
| 3-M41 | | | | | | | |
| VEGFA- | 220 | 225 | 8.51 | 10.48 | 9.46 | 9.48 ± 0.99 | 0.4 |
| 3-M42 | | | | | | | |
| VEGFA- | 218 | 225 | 18.94 | 21.26 | 23.1 | 21.1 ± 2.08 | 1.0 |
| 3-M43 | | | | | | | |
| VEGFA- | 216 | 225 | 20.15 | 21.88 | 24.09 | 22.04 ± 1.97 | 1.0 |
| 3-M44 | | | | | | | |
| VEGFA- | 214 | 225 | 20.78 | 26.17 | 25.27 | 24.07 ± 2.89 | 1.1 |
| 3-M45 | | | | | | | |
| VEGFA- | 212 | 225 | 24.04 | 22.52 | 23.59 | 23.38 ± 0.78 | 1.1 |
| 3-M46 | | | | | | | |
| VEGFA- | 208 | 225 | 16.63 | 17.4 | 18.33 | 17.45 ± 0.85 | 0.8 |
| 3-M48 | | | | | | | |
| VEGFA- | 206 | 225 | 14.97 | 20.64 | 17.55 | 17.72 ± 2.84 | 0.8 |
| 3-M49 | | | | | | | |
| VEGFA- | 204 | 225 | 0.2 | 0.29 | 0.39 | 0.29 ± 0.1 | 0.0 |
| 3-M50 | | | | | | | |
| VEGFA- | 202 | 225 | 0.41 | 0.33 | 0.41 | 0.38 ± 0.05 | 0.0 |
| 3-M51 | | | | | | | |
| VEGFA-3 | — | — | 21.38 | 22.08 | 21.95 | 21.8 ± 0.37 | 1.0 |
| (WT) | | | | | | | |

See Table 17 for the OmegaRNA sequence of each construct. A 20-nt sequence targeting VEGFA-3 (Table 16) was used as the variable region of the OmegaRNA. EboIscB-REC with 3 additional point mutations (pEDIT152) was used in this experiment.

TABLE 13
Editing efficiency of EboIscB in human cells
with combinatory engineered OmegaRNA.

| | | | | | | | Relative |
|---|---|---|---|---|---|---|---|
| Table 13 | | Length | Rep1 | Rep2 | Rep3 | Average | activity to |
| Target | OmegaRNA | (nt) | (%) | (%) | (%) | activity (%) | WT |
| VEGFA-3 | WT | 225 | 5.47 | 5.31 | 5.68 | 5.49 ± 0.19 | 1 |
| | M71 | 153 | 7.48 | 7.8 | 8.61 | 7.96 ± 0.58 | 1.45 |
| | M72 | 146 | 4.86 | 5.61 | 5.31 | 5.26 ± 0.38 | 0.96 |
| | M73 | 147 | 4.83 | 4.89 | 5.61 | 5.11 ± 0.43 | 0.93 |
| AAVS1-2 | WT | 225 | 2.03 | 1.93 | 2.18 | 2.05 ± 0.13 | 1 |
| | M71 | 153 | 3.81 | 3.54 | 3.78 | 3.71 ± 0.15 | 1.81 |
| | M72 | 146 | 2.07 | 2.13 | 2.05 | 2.08 ± 0.04 | 1.02 |
| | M73 | 147 | 2.4 | 2.15 | 2.03 | 2.19 ± 0.19 | 1.07 |
| HPRT-12 | WT | 225 | 1.09 | 1.57 | 1.11 | 1.26 ± 0.27 | 1 |
| | M71 | 153 | 3.27 | 3.88 | 2.89 | 3.35 ± 0.5 | 2.66 |
| | M72 | 146 | 1.4 | 1.68 | 1.61 | 1.56 ± 0.15 | 1.24 |
| | M73 | 147 | 1.58 | 1.89 | 1.74 | 1.74 ± 0.16 | 1.38 |

See Table 18 for the sequence of each engineered OmegaRNA. 20-nt sequence targeting each genomic locus (Table 16) was used as the variable region of the OmegaRNA. EboIscB-REC with 3 additional point mutations (pEDIT152) was used in this experiment.

TABLE 14
Editing efficiency of WT- and IscB-Triple mutant with WT or M71 OmegaRNA over 3 target sites.

| Table 14 | | WT OmegaRNA | | | M71 OmegaRNA | | | Relative |
|---|---|---|---|---|---|---|---|---|
| Target | | | | Average | | | Average | activity |
| site | Nuclease | Rep1(%) | Rep2(%) | (%) | Rep1(%) | Rep2(%) | (%) | (M71/WT) |
| VEGFA-3 | WT- | 16.79 | 15.11 | 15.95 ± 1.19 | 27.02 | 25.78 | 26.4 ± 0.88 | 1.66 |
| AAVS1-2 | IscB- | 4.22 | 3.67 | 3.95 ± 0.39 | 8.3 | 8.39 | 8.35 ± 0.06 | 2.12 |
| HPRT-12 | REC | 1.14 | 1.53 | 1.34 ± 0.28 | 6.2 | 6.59 | 6.4 ± 0.28 | 4.79 |
| | (pEDIT71) | | | | | | | |
| VEGFA-3 | IscB- | 21.2 | 22.24 | 21.72 ± 0.74 | 33.52 | 27.56 | 30.54 ± 4.21 | 1.41 |
| AAVS1-2 | REC- | 7.48 | 7.13 | 7.31 ± 0.25 | 13.27 | 11.29 | 12.28 ± 1.4 | 1.68 |
| HPRT-12 | Triple | 4.98 | 3.76 | 4.37 ± 0.86 | 8.87 | 11.39 | 10.13 ± 1.78 | 2.32 |
| | mutant | | | | | | | |
| | (pEDIT152) | | | | | | | |

TABLE 15
Editing efficiency of EbolscB in human cells using M71 OmegaRNA
over additional target sites. Note: EboIscB-REC with 7 additional point mutations (pEDIT420)
was used in this experiment.

| | Variable | WT OmegaRNA | | | | M71 OmegaRNA | | | | Relative |
|---|---|---|---|---|---|---|---|---|---|---|
| Target | region | Rep1 | Rep2 | Rep3 | Average | Rep1 | Rep2 | Rep3 | Average | activity |
| site | (20-nt) | (%) | (%) | (%) | (%) | (%) | (%) | (%) | (%) | (M71/WT) |
| FXN_1 | GGAGT | 23.79 | 27.78 | 24.89 | 25.49 ± 2.06 | 28.23 | 29.66 | 26.31 | 28.07 ± 1.68 | 1.1 |
| | GGGGC | | | | | | | | | |
| | AGAAT | | | | | | | | | |
| | CTGGA | | | | | | | | | |
| | (SEQ ID | | | | | | | | | |
| | NO: | | | | | | | | | |
| | 220) | | | | | | | | | |
| FXN_2 | TTGTTT | 2.8 | 3.79 | 2.75 | 3.11 ± 0.59 | 5.49 | 6.23 | 5.27 | 5.66 ± 0.5 | 1.82 |
| | TCTGT | | | | | | | | | |
| | AGGGA | | | | | | | | | |
| | GAAG | | | | | | | | | |
| | (SEQ ID | | | | | | | | | |
| | NO: | | | | | | | | | |
| | 221) | | | | | | | | | |
| FXN_14 | TACCA | 30.48 | 37.34 | 28.53 | 32.12 ± 4.63 | 29.33 | 29.1 | 28.81 | 29.08 ± 0.26 | 0.91 |
| | GCCAC | | | | | | | | | |
| | TGGGT | | | | | | | | | |
| | GGCTT | | | | | | | | | |
| | (SEQ ID | | | | | | | | | |
| | NO: | | | | | | | | | |
| | 222) | | | | | | | | | |
| FXN_15 | CTATC | 22.66 | 26.48 | 17.96 | 22.37 ± 4.27 | 25.71 | 25.39 | 27 | 26.03 ± 0.85 | 1.16 |
| | CAGCA | | | | | | | | | |
| | GTGAG | | | | | | | | | |
| | AACAA | | | | | | | | | |
| | (SEQ ID | | | | | | | | | |
| | NO: | | | | | | | | | |
| | 223) | | | | | | | | | |
| FXN_16 | CAAAC | 24.32 | 27.31 | 20.63 | 24.09 ± 3.35 | 26.94 | 26.54 | 26.5 | 26.66 ± 0.24 | 1.11 |
| | CTCAC | | | | | | | | | |
| | AACCG | | | | | | | | | |
| | TGAGC | | | | | | | | | |
| | (SEQ ID | | | | | | | | | |
| | NO: | | | | | | | | | |
| | 224) | | | | | | | | | |
| FXN_18 | GAAAT | 24.01 | 30.37 | 20.13 | 24.84 ± 5.17 | 36.66 | 39.83 | 35.78 | 37.42 ± 2.13 | 1.51 |
| | AACCA | | | | | | | | | |
| | AGAAA | | | | | | | | | |
| | AAAGC | | | | | | | | | |
| | (SEQ ID | | | | | | | | | |
| | NO: | | | | | | | | | |
| | 225) | | | | | | | | | |
| FXN_21 | TGAAG | 26.87 | 35.34 | 23.94 | 28.72 ± 5.92 | 39.19 | 42.27 | 42.17 | 41.21 ± 1.75 | 1.44 |
| | ACTGA | | | | | | | | | |
| | GTCCA | | | | | | | | | |
| | TTTAC | | | | | | | | | |
| | (SEQ ID | | | | | | | | | |
| | NO: | | | | | | | | | |
| | 226) | | | | | | | | | |

TABLE 16
Truncated variable region of OmegaRNA for each genomic target. For
facilitate efficient RNA transcription under human U6 promoter, additional G is appended
to the 5′-end if the initial nucleotide is not a G. This can cause duplication during
truncation whenever there is a G in the variable region, which are bolded in the table.

| | | Length | | |
|---|---|---|---|---|
| | | of | | |
| Genomic | | variable | | Truncated target |
| Target | Target | region | Truncated target | with additional G if applicable |
| VEGFA- | GGCCAG | 12 | TCCTAGCCTTAT (SEQ ID NO: | gTCCTAGCCTTAT (SEQ ID NO: |
| 3 | GTTGTA | | 228) | 229) |
| | ATCCTA | 13 | ATCCTAGCCTTAT (SEQ ID NO: | gATCCTAGCCTTAT (SEQ ID NO: |
| | GCCTTA | | 230) | 231) |
| | T (SEQ | 14 | AATCCTAGCCTTAT (SEQ ID NO: | gAATCCTAGCCTTAT (SEQ ID |
| | ID NO: | | 232) | NO: 233) |
| | 227) | 15 | TAATCCTAGCCTTAT (SEQ ID | gTAATCCTAGCCTTAT (SEQ ID |
| | | | NO: 234) | NO: 235) |
| | | 16 | GTAATCCTAGCCTTAT (SEQ ID | GTAATCCTAGCCTTAT (SEQ |
| | | | NO: 235) | ID NO: 235) |
| | | 17 | TGTAATCCTAGCCTTAT (SEQ ID | gTGTAATCCTAGCCTTAT (SEQ |
| | | | NO: 236) | ID NO: 237) |
| | | 18 | TTGTAATCCTAGCCTTAT (SEQ | gTTGTAATCCTAGCCTTAT |
| | | | ID NO: 238) | (SEQ ID NO: 239) |
| | | 19 | GTTGTAATCCTAGCCTTAT (SEQ | GTTGTAATCCTAGCCTTAT |
| | | | ID NO: 239) | (SEQ ID NO: 239) |
| | | 20 | GGTTGTAATCCTAGCCTTAT | GGTTGTAATCCTAGCCTTAT |
| | | | (SEQ ID NO: 240) | (SEQ ID NO: 240) |
| | | 21 | AGGTTGTAATCCTAGCCTTAT | gAGGTTGTAATCCTAGCCTTAT |
| | | | (SEQ ID NO: 241) | (SEQ ID NO: 242) |
| | | 22 | CAGGTTGTAATCCTAGCCTTAT | gCAGGTTGTAATCCTAGCCTTA |
| | | | (SEQ ID NO: 243) | T (SEQ ID NO: 244) |
| | | 23 | CCAGGTTGTAATCCTAGCCTTA | gCCAGGTTGTAATCCTAGCCT |
| | | | T (SEQ ID NO: 245) | TAT (SEQ ID NO: 246) |
| | | 24 | GCCAGGTTGTAATCCTAGCCTT | GCCAGGTTGTAATCCTAGCCT |
| | | | AT (SEQ ID NO: 246) | TAT (SEQ ID NO: 246) |
| | | 25 | GGCCAGGTTGTAATCCTAGCCT | GGCCAGGTTGTAATCCTAGCCT |
| | | | TAT (SEQ ID NO: 227) | TAT (SEQ ID NO: 227) |
| AAVS1- | CTATCG | 12 | AACTGGGTGGAA (SEQ ID NO: | gAACTGGGTGGAA (SEQ ID NO: |
| 2 | CCATGA | | 248) | 249) |
| | CAACTG | 13 | CAACTGGGTGGAA (SEQ ID NO: | gCAACTGGGTGGAA (SEQ ID |
| | GGTGGA | | 250) | NO: 251) |
| | A (SEQ | 14 | ACAACTGGGTGGAA (SEQ ID | gACAACTGGGTGGAA (SEQ ID |
| | ID NO: | | NO: 252) | NO: 253) |
| | 247) | 15 | GACAACTGGGTGGAA (SEQ ID | GACAACTGGGTGGAA (SEQ ID |
| | | | NO: 253) | NO: 253) |
| | | 16 | TGACAACTGGGTGGAA (SEQ ID | gTGACAACTGGGTGGAA (SEQ |
| | | | NO: 254) | ID NO: 255) |
| | | 17 | ATGACAACTGGGTGGAA (SEQ | gATGACAACTGGGTGGAA (SEQ |
| | | | ID NO: 256) | ID NO: 257) |
| | | 18 | CATGACAACTGGGTGGAA (SEQ | gCATGACAACTGGGTGGAA |
| | | | ID NO: 258) | (SEQ ID NO: 259) |
| | | 19 | CCATGACAACTGGGTGGAA | gCCATGACAACTGGGTGGAA |
| | | | (SEQ ID NO: 260) | (SEQ ID NO: 217) |
| | | 20 | GCCATGACAACTGGGTGGAA | GCCATGACAACTGGGTGGAA |
| | | | (SEQ ID NO: 217) | (SEQ ID NO: 217) |
| | | 21 | CGCCATGACAACTGGGTGGAA | gCGCCATGACAACTGGGTGGAA |
| | | | (SEQ ID NO: 261) | (SEQ ID NO: 262) |
| | | 22 | TCGCCATGACAACTGGGTGGA | gTCGCCATGACAACTGGGTGGA |
| | | | A (SEQ ID NO: 263) | A (SEQ ID NO: 264) |
| | | 23 | ATCGCCATGACAACTGGGTGG | gATCGCCATGACAACTGGGTGG |
| | | | AA (SEQ ID NO: 265) | AA (SEQ ID NO: 266) |
| | | 24 | TATCGCCATGACAACTGGGTGG | gTATCGCCATGACAACTGGGTG |
| | | | AA (SEQ ID NO: 267) | GAA (SEQ ID NO: 268) |
| | | 25 | CTATCGCCATGACAACTGGGTG | gCTATCGCCATGACAACTGGGT |
| | | | GAA (SEQ ID NO: 247) | GGAA (SEQ ID NO: 269) |
| HPRT- | GGAAAT | 12 | AATCCCTAAACT (SEQ ID NO: | gAATCCCTAAACT (SEQ ID NO: |
| 7 | ACAATC | | 270 | 271) |
| | CCTAAA | 13 | CAATCCCTAAACT (SEQ ID NO: | gCAATCCCTAAACT (SEQ ID NO: |
| | CT (SEQ | | 272) | 273) |
| | ID NO: | 14 | ACAATCCCTAAACT (SEQ ID | gACAATCCCTAAACT (SEQ ID |
| | 177) | | NO: 274) | NO: 275) |
| | | 15 | TACAATCCCTAAACT (SEQ ID | gTACAATCCCTAAACT (SEQ ID |
| | | | NO: 276) | NO: 277) |
| | | 16 | ATACAATCCCTAAACT (SEQ ID | gATACAATCCCTAAACT (SEQ ID |
| | | | NO: 278) | NO: 279) |
| | | 17 | AATACAATCCCTAAACT (SEQ | gAATACAATCCCTAAACT (SEQ |
| | | | ID NO: 280) | ID NO: 281) |
| | | 18 | AAATACAATCCCTAAACT (SEQ | gAAATACAATCCCTAAACT |
| | | | ID NO: 282) | (SEQ ID NO: 283) |
| | | 19 | GAAATACAATCCCTAAACT | GAAATACAATCCCTAAACT |
| | | | (SEQ ID NO: 283) | (SEQ ID NO: 283) |
| | | 20 | GGAAATACAATCCCTAAACT | GGAAATACAATCCCTAAACT |
| | | | (SEQ ID NO: 177) | (SEQ ID NO: 177) |
| HPRT- | TGAACT | 12 | TATCTGTAAGAA (SEQ ID NO: | gTATCTGTAAGAA (SEQ ID NO: |
| 12 | CATATC | | 284) | 285) |
| | TGTAAG | 13 | ATATCTGTAAGAA (SEQ ID NO: | gATATCTGTAAGAA (SEQ ID |
| | AA (SEQ | | 286) | NO: 287) |
| | ID NO: | 14 | CATATCTGTAAGAA (SEQ ID | gCATATCTGTAAGAA (SEQ ID |
| | 182) | | NO: 288) | NO: 289) |
| | | 15 | TCATATCTGTAAGAA (SEQ ID | gTCATATCTGTAAGAA (SEQ ID |
| | | | NO: 290) | NO: 291) |
| | | 16 | CTCATATCTGTAAGAA (SEQ ID | gCTCATATCTGTAAGAA (SEQ ID |
| | | | NO: 292) | NO: 293) |
| | | 17 | ACTCATATCTGTAAGAA (SEQ | gACTCATATCTGTAAGAA (SEQ |
| | | | ID NO: 294) | ID NO: 295) |
| | | 18 | AACTCATATCTGTAAGAA (SEQ | gAACTCATATCTGTAAGAA |
| | | | ID NO: 296) | (SEQ ID NO: 297) |
| | | 19 | GAACTCATATCTGTAAGAA | GAACTCATATCTGTAAGAA |
| | | | (SEQ ID NO: 297) | (SEQ ID NO: 297) |
| | | 20 | TGAACTCATATCTGTAAGAA | gTGAACTCATATCTGTAAGAA |
| | | | (SEQ ID NO: 182) | (SEQ ID NO: 298) |

TABLE 17
Various OmegaRNA constructs used in Table 7-11. The 20-nt variable
sequence at the 5′-end the OmegaRNA is shown as (N20).

| | Trunc- | Trunc- | | | | |
|---|---|---|---|---|---|---|
| Omega | ation | ation | | Structural | | |
| RNA | start | end | Linker | region | Length | OmegaRNA sequence (in DNA) |
| WT | — | — | — | — | 225 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTTACAACAAATAAGGAGTTATT |
| | | | | | | AG (SEQ ID NO: 299) |
| M1 | 32 | 77 | GAAAA | P1 Loop | 189 | (N20)GTCAATAACCCGAAAAGGGTTA |
| | | | | | | CTGAGCAGAGCAGTGACATGTCATTC |
| | | | | | | GCCGCGGGGTGATTCCAAGCTCCGC |
| | | | | | | GCTCCGGCTAGACATGCCCATGCTAT |
| | | | | | | GGAAACTTTAACGGTATGTGCGGTTT |
| | | | | | | TCCGCTCATACCGGCTTACAACAAAT |
| | | | | | | AAGGAGTTATTAG (SEQ ID NO: 300) |
| M2 | 34 | 75 | GAAAA | | 193 | (N20)GTCAATAACCCATGAAAAAAGG |
| | | | | | | GTTACTGAGCAGAGCAGTGACATGT |
| | | | | | | CATTCGCCGCGGGGTGATTCCAAGCT |
| | | | | | | CCGCGCTCCGGCTAGACATGCCCATG |
| | | | | | | CTATGGAAACTTTAACGGTATGTGCG |
| | | | | | | GTTTTCCGCTCATACCGGCTTACAAC |
| | | | | | | AAATAAGGAGTTATTAG (SEQ ID NO: |
| | | | | | | 301) |
| M3 | 36 | 74 | GAAAA | | 196 | (N20)GTCAATAACCCATGAGAAAAAA |
| | | | | | | AGGGTTACTGAGCAGAGCAGTGACA |
| | | | | | | TGTCATTCGCCGCGGGGTGATTCCAA |
| | | | | | | GCTCCGCGCTCCGGCTAGACATGCCC |
| | | | | | | ATGCTATGGAAACTTTAACGGTATGT |
| | | | | | | GCGGTTTTCCGCTCATACCGGCTTAC |
| | | | | | | AACAAATAAGGAGTTATTAG (SEQ ID |
| | | | | | | NO: 302) |
| M4 | 38 | 72 | GAAAA | | 200 | (N20)GTCAATAACCCATGACTGAAAA |
| | | | | | | AGAAAGGGTTACTGAGCAGAGCAGT |
| | | | | | | GACATGTCATTCGCCGCGGGGTGATT |
| | | | | | | CCAAGCTCCGCGCTCCGGCTAGACAT |
| | | | | | | GCCCATGCTATGGAAACTTTAACGGT |
| | | | | | | ATGTGCGGTTTTCCGCTCATACCGGC |
| | | | | | | TTACAACAAATAAGGAGTTATTAG |
| | | | | | | (SEQ ID NO: 303) |
| M5 | 40 | 70 | GAAAA | | 204 | (N20)GTCAATAACCCATGACTGAGAA |
| | | | | | | AAGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTTACAACAAATAAGGAGTTATT |
| | | | | | | AG (SEQ ID NO: 304) |
| M6 | 42 | 68 | GAAAA | | 208 | (N20)GTCAATAACCCATGACTGAAGG |
| | | | | | | AAAATGGAAGAAAGGGTTACTGAGC |
| | | | | | | AGAGCAGTGACATGTCATTCGCCGC |
| | | | | | | GGGGTGATTCCAAGCTCCGCGCTCCG |
| | | | | | | GCTAGACATGCCCATGCTATGGAAA |
| | | | | | | CTTTAACGGTATGTGCGGTTTTCCGC |
| | | | | | | TCATACCGGCTTACAACAAATAAGG |
| | | | | | | AGTTATTAG (SEQ ID NO: 305) |
| M7 | 44 | 66 | GAAAA | | 212 | (N20)GTCAATAACCCATGACTGAAGT |
| | | | | | | CGAAAAGATGGAAGAAAGGGTTACT |
| | | | | | | GAGCAGAGCAGTGACATGTCATTCG |
| | | | | | | CCGCGGGGTGATTCCAAGCTCCGCGC |
| | | | | | | TCCGGCTAGACATGCCCATGCTATGG |
| | | | | | | AAACTTTAACGGTATGTGCGGTTTTC |
| | | | | | | CGCTCATACCGGCTTACAACAAATAA |
| | | | | | | GGAGTTATTAG (SEQ ID NO: 306) |
| M8 | 46 | 65 | GAAAA | | 215 | (N20)GTCAATAACCCATGACTGAAGT |
| | | | | | | CATGAAAATGATGGAAGAAAGGGTT |
| | | | | | | ACTGAGCAGAGCAGTGACATGTCAT |
| | | | | | | TCGCCGCGGGGTGATTCCAAGCTCCG |
| | | | | | | CGCTCCGGCTAGACATGCCCATGCTA |
| | | | | | | TGGAAACTTTAACGGTATGTGCGGTT |
| | | | | | | TTCCGCTCATACCGGCTTACAACAAA |
| | | | | | | TAAGGAGTTATTAG (SEQ ID NO: 307) |
| M9 | 48 | 63 | GAAAA | | 219 | (N20)GTCAATAACCCATGACTGAAGT |
| | | | | | | CATGGGAAAACCTGATGGAAGAAAG |
| | | | | | | GGTTACTGAGCAGAGCAGTGACATG |
| | | | | | | TCATTCGCCGCGGGGTGATTCCAAGC |
| | | | | | | TCCGCGCTCCGGCTAGACATGCCCAT |
| | | | | | | GCTATGGAAACTTTAACGGTATGTGC |
| | | | | | | GGTTTTCCGCTCATACCGGCTTACAA |
| | | | | | | CAAATAAGGAGTTATTAG (SEQ ID |
| | | | | | | NO: 308) |
| M10 | 50 | 61 | GAAAA | | 223 | (N20)GTCAATAACCCATGACTGAAGT |
| | | | | | | CATGGGCGAAAAGTCCTGATGGAAG |
| | | | | | | AAAGGGTTACTGAGCAGAGCAGTGA |
| | | | | | | CATGTCATTCGCCGCGGGGTGATTCC |
| | | | | | | AAGCTCCGCGCTCCGGCTAGACATGC |
| | | | | | | CCATGCTATGGAAACTTTAACGGTAT |
| | | | | | | GTGCGGTTTTCCGCTCATACCGGCTT |
| | | | | | | ACAACAAATAAGGAGTTATTAG (SEQ |
| | | | | | | ID NO: 309) |
| M11 | 52 | 59 | GAAAA | | 227 | (N20)GTCAATAACCCATGACTGAAGT |
| | | | | | | CATGGGCTTGAAAAAGGTCCTGATG |
| | | | | | | GAAGAAAGGGTTACTGAGCAGAGCA |
| | | | | | | GTGACATGTCATTCGCCGCGGGGTGA |
| | | | | | | TTCCAAGCTCCGCGCTCCGGCTAGAC |
| | | | | | | ATGCCCATGCTATGGAAACTTTAACG |
| | | | | | | GTATGTGCGGTTTTCCGCTCATACCG |
| | | | | | | GCTTACAACAAATAAGGAGTTATTA |
| | | | | | | G (SEQ ID NO: 310) |
| M25 | 32 | 77 | GAAA | | 188 | (N20)GTCAATAACCCGAAAGGGTTAC |
| | | | | | | TGAGCAGAGCAGTGACATGTCATTC |
| | | | | | | GCCGCGGGGTGATTCCAAGCTCCGC |
| | | | | | | GCTCCGGCTAGACATGCCCATGCTAT |
| | | | | | | GGAAACTTTAACGGTATGTGCGGTTT |
| | | | | | | TCCGCTCATACCGGCTTACAACAAAT |
| | | | | | | AAGGAGTTATTAG (SEQ ID NO: 311) |
| M26 | 34 | 75 | GAAA | | 192 | (N20)GTCAATAACCCATGAAAAAGGG |
| | | | | | | TTACTGAGCAGAGCAGTGACATGTC |
| | | | | | | ATTCGCCGCGGGGTGATTCCAAGCTC |
| | | | | | | CGCGCTCCGGCTAGACATGCCCATGC |
| | | | | | | TATGGAAACTTTAACGGTATGTGCGG |
| | | | | | | TTTTCCGCTCATACCGGCTTACAACA |
| | | | | | | AATAAGGAGTTATTAG (SEQ ID NO: |
| | | | | | | 312) |
| M27 | 36 | 74 | GAAA | | 195 | (N20)GTCAATAACCCATGAGAAAAAA |
| | | | | | | GGGTTACTGAGCAGAGCAGTGACAT |
| | | | | | | GTCATTCGCCGCGGGGTGATTCCAAG |
| | | | | | | CTCCGCGCTCCGGCTAGACATGCCCA |
| | | | | | | TGCTATGGAAACTTTAACGGTATGTG |
| | | | | | | CGGTTTTCCGCTCATACCGGCTTACA |
| | | | | | | ACAAATAAGGAGTTATTAG (SEQ ID |
| | | | | | | NO: 313) |
| M28 | 38 | 72 | GAAA | | 199 | (N20)GTCAATAACCCATGACTGAAAA |
| | | | | | | GAAAGGGTTACTGAGCAGAGCAGTG |
| | | | | | | ACATGTCATTCGCCGCGGGGTGATTC |
| | | | | | | CAAGCTCCGCGCTCCGGCTAGACATG |
| | | | | | | CCCATGCTATGGAAACTTTAACGGTA |
| | | | | | | TGTGCGGTTTTCCGCTCATACCGGCT |
| | | | | | | TACAACAAATAAGGAGTTATTAG |
| | | | | | | (SEQ ID NO: 314) |
| M29 | 40 | 70 | GAAA | | 203 | (N20)GTCAATAACCCATGACTGAGAA |
| | | | | | | AGAAGAAAGGGTTACTGAGCAGAGC |
| | | | | | | AGTGACATGTCATTCGCCGCGGGGTG |
| | | | | | | ATTCCAAGCTCCGCGCTCCGGCTAGA |
| | | | | | | CATGCCCATGCTATGGAAACTTTAAC |
| | | | | | | GGTATGTGCGGTTTTCCGCTCATACC |
| | | | | | | GGCTTACAACAAATAAGGAGTTATT |
| | | | | | | AG (SEQ ID NO: 315) |
| M30 | 42 | 68 | GAAA | | 207 | (N20)GTCAATAACCCATGACTGAAGG |
| | | | | | | AAATGGAAGAAAGGGTTACTGAGCA |
| | | | | | | GAGCAGTGACATGTCATTCGCCGCG |
| | | | | | | GGGTGATTCCAAGCTCCGCGCTCCGG |
| | | | | | | CTAGACATGCCCATGCTATGGAAACT |
| | | | | | | TTAACGGTATGTGCGGTTTTCCGCTC |
| | | | | | | ATACCGGCTTACAACAAATAAGGAG |
| | | | | | | TTATTAG (SEQ ID NO: 316) |
| M31 | 44 | 66 | GAAA | | 211 | (N20)GTCAATAACCCATGACTGAAGT |
| | | | | | | CGAAAGATGGAAGAAAGGGTTACTG |
| | | | | | | AGCAGAGCAGTGACATGTCATTCGC |
| | | | | | | CGCGGGGTGATTCCAAGCTCCGCGCT |
| | | | | | | CCGGCTAGACATGCCCATGCTATGGA |
| | | | | | | AACTTTAACGGTATGTGCGGTTTTCC |
| | | | | | | GCTCATACCGGCTTACAACAAATAA |
| | | | | | | GGAGTTATTAG (SEQ ID NO: 317) |
| M32 | 46 | 65 | GAAA | | 214 | (N20)GTCAATAACCCATGACTGAAGT |
| | | | | | | CATGAAATGATGGAAGAAAGGGTTA |
| | | | | | | CTGAGCAGAGCAGTGACATGTCATTC |
| | | | | | | GCCGCGGGGTGATTCCAAGCTCCGC |
| | | | | | | GCTCCGGCTAGACATGCCCATGCTAT |
| | | | | | | GGAAACTTTAACGGTATGTGCGGTTT |
| | | | | | | TCCGCTCATACCGGCTTACAACAAAT |
| | | | | | | AAGGAGTTATTAG (SEQ ID NO: 318) |
| M33 | 48 | 63 | GAAA | | 218 | (N20)GTCAATAACCCATGACTGAAGT |
| | | | | | | CATGGGAAACCTGATGGAAGAAAGG |
| | | | | | | GTTACTGAGCAGAGCAGTGACATGT |
| | | | | | | CATTCGCCGCGGGGTGATTCCAAGCT |
| | | | | | | CCGCGCTCCGGCTAGACATGCCCATG |
| | | | | | | CTATGGAAACTTTAACGGTATGTGCG |
| | | | | | | GTTTTCCGCTCATACCGGCTTACAAC |
| | | | | | | AAATAAGGAGTTATTAG (SEQ ID NO: |
| | | | | | | 319) |
| M34 | 50 | 61 | GAAA | | 222 | (N20)GTCAATAACCCATGACTGAAGT |
| | | | | | | CATGGGCGAAAGTCCTGATGGAAGA |
| | | | | | | AAGGGTTACTGAGCAGAGCAGTGAC |
| | | | | | | ATGTCATTCGCCGCGGGGTGATTCCA |
| | | | | | | AGCTCCGCGCTCCGGCTAGACATGCC |
| | | | | | | CATGCTATGGAAACTTTAACGGTATG |
| | | | | | | TGCGGTTTTCCGCTCATACCGGCTTA |
| | | | | | | CAACAAATAAGGAGTTATTAG (SEQ |
| | | | | | | ID NO: 320) |
| M35 | 52 | 59 | GAAA | | 226 | (N20)GTCAATAACCCATGACTGAAGT |
| | | | | | | CATGGGCTTGAAAAGGTCCTGATGG |
| | | | | | | AAGAAAGGGTTACTGAGCAGAGCAG |
| | | | | | | TGACATGTCATTCGCCGCGGGGTGAT |
| | | | | | | TCCAAGCTCCGCGCTCCGGCTAGACA |
| | | | | | | TGCCCATGCTATGGAAACTTTAACGG |
| | | | | | | TATGTGCGGTTTTCCGCTCATACCGG |
| | | | | | | CTTACAACAAATAAGGAGTTATTAG |
| | | | | | | (SEQ ID NO: 321) |
| M40 | 224 | 225 | | 3′-end | 228 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTTACAACAAATAAGGAGTTATT |
| | | | | | | (SEQ ID NO: 322) |
| M41 | 222 | 225 | | | 226 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTTACAACAAATAAGGAGTTA |
| | | | | | | (SEQ ID NO: 323) |
| M42 | 220 | 225 | | | 224 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTTACAACAAATAAGGAGT (SEQ |
| | | | | | | ID NO: 324) |
| M43 | 218 | 225 | | | 222 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTTACAACAAATAAGGA (SEQ ID |
| | | | | | | NO: 325) |
| M44 | 216 | 225 | | | 220 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTTACAACAAATAAG (SEQ ID |
| | | | | | | NO: 326) |
| M45 | 214 | 225 | | | 218 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTTACAACAAATA (SEQ ID NO: |
| | | | | | | 327) |
| M46 | 212 | 225 | | | 216 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTTACAACAAA (SEQ ID NO: 328) |
| M47 | 210 | 225 | | | 214 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTTACAACA (SEQ ID NO: 329) |
| M48 | 208 | 225 | | | 212 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTTACAA (SEQ ID NO: 330) |
| M49 | 206 | 225 | | | 210 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTTAC (SEQ ID NO: 331) |
| M50 | 204 | 225 | | | 208 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGCTT (SEQ ID NO: 332) |
| M51 | 202 | 225 | | | 206 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGTTTTCCGCTCATAC |
| | | | | | | CGGC (SEQ ID NO: 333) |
| M52 | 179 | 192 | GAAA | P5 Loop | 220 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGGAAACATACCGGCTTACA |
| | | | | | | ACAAATAAGGAGTTATTAG (SEQ ID |
| | | | | | | NO: 334) |
| M53 | 180 | 191 | GAAA | | 222 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGAAATCATACCGGCTTAC |
| | | | | | | AACAAATAAGGAGTTATTAG (SEQ ID |
| | | | | | | NO: 335) |
| M54 | 181 | 190 | GAAA | | 224 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGGAAACTCATACCGGCTT |
| | | | | | | ACAACAAATAAGGAGTTATTAG (SEQ |
| | | | | | | ID NO: 336) |
| M55 | 183 | 188 | GAAA | | 228 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGTGCGGAAACGCTCATACC |
| | | | | | | GGCTTACAACAAATAAGGAGTTATT |
| | | | | | | AG (SEQ ID NO: 337) |
| M56 | 178 | 193 | GAAA | | 218 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTATGAAAATACCGGCTTACAAC |
| | | | | | | AAATAAGGAGTTATTAG (SEQ ID NO: |
| | | | | | | 338) |
| M57 | 176 | 195 | GAAA | | 214 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGTGAAAACCGGCTTACAACAAAT |
| | | | | | | AAGGAGTTATTAG (SEQ ID NO: 339) |
| M58 | 174 | 197 | GAAA | | 210 | (N20)GTCAATAACCCATGACTGAAGT |
| | | | | | | CATGGGCTTGCAGATGCAGGTCCTGA |
| | | | | | | TGGAAGAAAGGGTTACTGAGCAGAG |
| | | | | | | CAGTGACATGTCATTCGCCGCGGGGT |
| | | | | | | GATTCCAAGCTCCGCGCTCCGGCTAG |
| | | | | | | ACATGCCCATGCTATGGAAACTTTAA |
| | | | | | | CGGAAACGGCTTACAACAAATAAGG |
| | | | | | | AGTTATTAG (SEQ ID NO: 340) |

TABLE 18
Combinatory engineering of OmegaRNA. The 20-nt variable sequence
at the 5′-end the OmegaRNA is shown as (N20).

| | P1 Loop | | | 3′-End | | P5 Loop | | | | |
|---|---|---|---|---|---|---|---|---|---|---|
| | Trunc- | Trunc- | | Trunc- | Trunc- | Trunc- | Trunc- | | OmegaRNA | |
| Omega | ation | ation | | ation | ation | ation | ation | | sequence | Length |
| RNA | start | end | Linker | start | end | start | end | Linker | (in DNA) | (nt) |
| M71 | 32 | 77 | GAAA | 212 | 225 | 176 | 195 | 32 | (N20)GTCAAT | 153 |
| | | | | | | | | | AACCCGAAA | |
| | | | | | | | | | GGGTTACTG | |
| | | | | | | | | | AGCAGAGCA | |
| | | | | | | | | | GTGACATGT | |
| | | | | | | | | | CATTCGCCG | |
| | | | | | | | | | CGGGGTGAT | |
| | | | | | | | | | TCCAAGCTC | |
| | | | | | | | | | CGCGCTCCG | |
| | | | | | | | | | GCTAGACAT | |
| | | | | | | | | | GCCCATGCT | |
| | | | | | | | | | ATGGAAACT | |
| | | | | | | | | | TTAACGGTG | |
| | | | | | | | | | AAAACCGGC | |
| | | | | | | | | | TTACAACAA | |
| | | | | | | | | | A (SEQ ID | |
| | | | | | | | | | NO: 341) | |
| M72 | 32 | 77 | GAAA | 205 | 225 | 176 | 195 | 32 | (N20)GTCAAT | 146 |
| | | | | | | | | | AACCCGAAA | |
| | | | | | | | | | GGGTTACTG | |
| | | | | | | | | | AGCAGAGCA | |
| | | | | | | | | | GTGACATGT | |
| | | | | | | | | | CATTCGCCG | |
| | | | | | | | | | CGGGGTGAT | |
| | | | | | | | | | TCCAAGCTC | |
| | | | | | | | | | CGCGCTCCG | |
| | | | | | | | | | GCTAGACAT | |
| | | | | | | | | | GCCCATGCT | |
| | | | | | | | | | ATGGAAACT | |
| | | | | | | | | | TTAACGGTG | |
| | | | | | | | | | AAAACCGGC | |
| | | | | | | | | | TTA (SEQ ID | |
| | | | | | | | | | NO: 342) | |
| M73 | 32 | 77 | GAAA | 206 | 225 | 176 | 195 | 32 | (N20)GTCAAT | 147 |
| | | | | | | | | | AACCCGAAA | |
| | | | | | | | | | GGGTTACTG | |
| | | | | | | | | | AGCAGAGCA | |
| | | | | | | | | | GTGACATGT | |
| | | | | | | | | | CATTCGCCG | |
| | | | | | | | | | CGGGGTGAT | |
| | | | | | | | | | TCCAAGCTC | |
| | | | | | | | | | CGCGCTCCG | |
| | | | | | | | | | GCTAGACAT | |
| | | | | | | | | | GCCCATGCT | |
| | | | | | | | | | ATGGAAACT | |
| | | | | | | | | | TTAACGGTG | |
| | | | | | | | | | AAAACCGGC | |
| | | | | | | | | | TTAC (SEQ | |
| | | | | | | | | | ID NO: 343) | |

TABLE 19
M71 variants for further engineering of OmegaRNA. The 20-nt variable sequence at
the 5′-end the OmegaRNA is shown as (N20)

| | Region | Modification | Goal | Final OmegaRNA sequence |
|---|---|---|---|---|
| 1 | P1 Stem- | Delete G1 | Reduce | (N20)TCAATAACCCGAAAGGGTTACTGAGCA |
| | Loop | | length | GAGCAGTGACATGTCATTCGCCGCGGGGTGA |
| | | | | TTCCAAGCTCCGCGCTCCGGCTAGACATGCC |
| | | | | CATGCTATGGAAACTTTAACGGTGAAAACCG |
| | | | | GCTTACAACAAA (SEQ ID NO: 344) |
| 2 | P1 Stem- | Trimming from | Reduce | (N20)GTCAATAACCGAAAGGTTACTGAGCAG |
| | Loop | Stem, 1-bp | length | AGCAGTGACATGTCATTCGCCGCGGGGTGAT |
| | | | | TCCAAGCTCCGCGCTCCGGCTAGACATGCCC |
| | | | | ATGCTATGGAAACTTTAACGGTGAAAACCGG |
| | | | | CTTACAACAAA (SEQ ID NO: 345) |
| 3 | P1 Stem- | Trimming from | Reduce | (N20)GTCAATAACGAAAGTTACTGAGCAGAG |
| | Loop | Stem, 2-bp | length | CAGTGACATGTCATTCGCCGCGGGGTGATTC |
| | | | | CAAGCTCCGCGCTCCGGCTAGACATGCCCAT |
| | | | | GCTATGGAAACTTTAACGGTGAAAACCGGCT |
| | | | | TACAACAAA (SEQ ID NO: 346) |
| 4 | P1 Stem- | Trimming from | Reduce | (N20)GTCAATAAGAAATTACTGAGCAGAGCA |
| | Loop | Stem, 3-bp | length | GTGACATGTCATTCGCCGCGGGGTGATTCCA |
| | | | | AGCTCCGCGCTCCGGCTAGACATGCCCATGC |
| | | | | TATGGAAACTTTAACGGTGAAAACCGGCTTA |
| | | | | CAACAAA (SEQ ID NO: 347) |
| 5 | P1 Stem- | Trimming from | Reduce | (N20)GTCAATAGAAATACTGAGCAGAGCAGT |
| | Loop | Stem, 4-bp | length | GACATGTCATTCGCCGCGGGGTGATTCCAAG |
| | | | | CTCCGCGCTCCGGCTAGACATGCCCATGCTA |
| | | | | TGGAAACTTTAACGGTGAAAACCGGCTTACA |
| | | | | ACAAA (SEQ ID NO: 348) |
| 6 | P1 Stem- | Trimming from | Reduce | (N20)GTCAATGAAAACTGAGCAGAGCAGTGA |
| | Loop | Stem, 5-bp | length | CATGTCATTCGCCGCGGGGTGATTCCAAGCT |
| | | | | CCGCGCTCCGGCTAGACATGCCCATGCTATG |
| | | | | GAAACTTTAACGGTGAAAACCGGCTTACAAC |
| | | | | AAA (SEQ ID NO: 349) |
| 7 | P1 Stem- | Trimming from | Reduce | (N20)GTCAAGAAACTGAGCAGAGCAGTGACA |
| | Loop | Stem, 6-bp | length | TGTCATTCGCCGCGGGGTGATTCCAAGCTCC |
| | | | | GCGCTCCGGCTAGACATGCCCATGCTATGGA |
| | | | | AACTTTAACGGTGAAAACCGGCTTACAACAA |
| | | | | A (SEQ ID NO: 350) |
| 8 | P1 Stem- | Trimming from | Reduce | (N20)GTCAGAAATGAGCAGAGCAGTGACATG |
| | Loop | Stem, 7-bp | length | TCATTCGCCGCGGGGTGATTCCAAGCTCCGC |
| | | | | GCTCCGGCTAGACATGCCCATGCTATGGAAA |
| | | | | CTTTAACGGTGAAAACCGGCTTACAACAAA |
| | | | | (SEQ ID NO: 351) |
| 9 | P1 Stem- | Trimming from | Reduce | (N20)GTCGAAAGAGCAGAGCAGTGACATGTC |
| | Loop | Stem, 8-bp | length | ATTCGCCGCGGGGTGATTCCAAGCTCCGCGC |
| | | | | TCCGGCTAGACATGCCCATGCTATGGAAACT |
| | | | | TTAACGGTGAAAACCGGCTTACAACAAA |
| | | | | (SEQ ID NO: 352) |
| 10 | P1 Stem- | Trimming from | Reduce | (N20)GTGAAAAGCAGAGCAGTGACATGTCAT |
| | Loop | Stem, 9-bp | length | TCGCCGCGGGGTGATTCCAAGCTCCGCGCTC |
| | | | | CGGCTAGACATGCCCATGCTATGGAAACTTT |
| | | | | AACGGTGAAAACCGGCTTACAACAAA (SEQ |
| | | | | ID NO: 353) |
| 11 | P1 Stem- | Trimming from | Reduce | (N20)GGAAAGCAGAGCAGTGACATGTCATTC |
| | Loop | Stem, 10-bp | length | GCCGCGGGGTGATTCCAAGCTCCGCGCTCCG |
| | | | | GCTAGACATGCCCATGCTATGGAAACTTTAA |
| | | | | CGGTGAAAACCGGCTTACAACAAA (SEQ ID |
| | | | | NO: 354) |
| 12 | P1 Stem- | Trimming from | Reduce | (N20)GTAATAACCCGAAAGGGTTACTAGCAG |
| | Loop | bottom of stem, | length | AGCAGTGACATGTCATTCGCCGCGGGGTGAT |
| | | 1-bp | | TCCAAGCTCCGCGCTCCGGCTAGACATGCCC |
| | | | | ATGCTATGGAAACTTTAACGGTGAAAACCGG |
| | | | | CTTACAACAAA (SEQ ID NO: 355) |
| 13 | P1 Stem- | Trimming from | Reduce | (N20)GTATAACCCGAAAGGGTTACAGCAGAG |
| | Loop | bottom of stem, | length | CAGTGACATGTCATTCGCCGCGGGGTGATTC |
| | | 2-bp | | CAAGCTCCGCGCTCCGGCTAGACATGCCCAT |
| | | | | GCTATGGAAACTTTAACGGTGAAAACCGGCT |
| | | | | TACAACAAA (SEQ ID NO: 356) |
| 14 | P1 Stem- | Trimming from | Reduce | (N20)GTTAACCCGAAAGGGTTAAGCAGAGCA |
| | Loop | bottom of stem, | length | GTGACATGTCATTCGCCGCGGGGTGATTCCA |
| | | 3-bp | | AGCTCCGCGCTCCGGCTAGACATGCCCATGC |
| | | | | TATGGAAACTTTAACGGTGAAAACCGGCTTA |
| | | | | CAACAAA (SEQ ID NO: 357) |
| 15 | P1 Stem- | Trimming from | Reduce | (N20)GTAACCCGAAAGGGTTAGCAGAGCAGT |
| | Loop | bottom of stem, | length | GACATGTCATTCGCCGCGGGGTGATTCCAAG |
| | | 4-bp | | CTCCGCGCTCCGGCTAGACATGCCCATGCTA |
| | | | | TGGAAACTTTAACGGTGAAAACCGGCTTACA |
| | | | | ACAAA (SEQ ID NO: 358) |
| 16 | P1 Stem- | Trimming from | Reduce | (N20)GTACCCGAAAGGGTAGCAGAGCAGTGA |
| | Loop | bottom of stem, | length | CATGTCATTCGCCGCGGGGTGATTCCAAGCT |
| | | 5-bp | | CCGCGCTCCGGCTAGACATGCCCATGCTATG |
| | | | | GAAACTTTAACGGTGAAAACCGGCTTACAAC |
| | | | | AAA (SEQ ID NO: 359) |
| 17 | P1 Stem- | Trimming from | Reduce | (N20)GTCCCGAAAGGGAGCAGAGCAGTGACA |
| | Loop | bottom of stem, | length | TGTCATTCGCCGCGGGGTGATTCCAAGCTCC |
| | | 6-bp | | GCGCTCCGGCTAGACATGCCCATGCTATGGA |
| | | | | AACTTTAACGGTGAAAACCGGCTTACAACAA |
| | | | | A (SEQ ID NO: 360) |
| 18 | P1 Stem- | Trimming from | Reduce | (N20)GTCCGAAAGGAGCAGAGCAGTGACATG |
| | Loop | bottom of stem, | length | TCATTCGCCGCGGGGTGATTCCAAGCTCCGC |
| | | 7-bp | | GCTCCGGCTAGACATGCCCATGCTATGGAAA |
| | | | | CTTTAACGGTGAAAACCGGCTTACAACAAA |
| | | | | (SEQ ID NO: 361) |
| 19 | P1 Stem- | Trimming from | Reduce | (N20)GTCGAAAGAGCAGAGCAGTGACATGTC |
| | Loop | bottom of stem, | length | ATTCGCCGCGGGGTGATTCCAAGCTCCGCGC |
| | | 8-bp | | TCCGGCTAGACATGCCCATGCTATGGAAACT |
| | | | | TTAACGGTGAAAACCGGCTTACAACAAA |
| | | | | (SEQ ID NO: 352) |
| 20 | P1 Stem- | Trimming from | Reduce | (N20)GTGAAAAGCAGAGCAGTGACATGTCAT |
| | Loop | bottom of stem, | length | TCGCCGCGGGGTGATTCCAAGCTCCGCGCTC |
| | | 9-bp | | CGGCTAGACATGCCCATGCTATGGAAACTTT |
| | | | | AACGGTGAAAACCGGCTTACAACAAA (SEQ |
| | | | | ID NO: 353) |
| 21 | P1 Stem- | Close C:A gap, | Increase | (N20)GTCAGTAACCCGAAAGGGTTACTGAGC |
| | Loop | C:G | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 362) |
| 22 | P1 Stem- | Close C:A gap, | Increase | (N20)GTCACTAACCCGAAAGGGTTAGTGAGC |
| | Loop | G:C | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 363) |
| 23 | P1 Stem- | Close C:A gap, | Increase | (N20)GTCATTAACCCGAAAGGGTTAATGAGC |
| | Loop | A:U | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 364) |
| 24 | P1 Stem- | Close C:A gap, | Increase | (N20)GTCAATAACCCGAAAGGGTTATTGAGC |
| | Loop | U:A | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 365) |
| 25 | P1 Stem- | Introduce G:C, | Increase | (N20)GGCAATAACCCGAAAGGGTTACTGCGC |
| | Loop | Position 1 | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 366) |
| 26 | P1 Stem- | Introduce C:G, | Increase | (N20)GCCAATAACCCGAAAGGGTTACTGGGC |
| | Loop | Position 1 | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 367) |
| 27 | P1Stem- | Introduce G:C, | Increase | (N20)GTCGATAACCCGAAAGGGTTACCGAGC |
| | Loop | Position 2 | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 368) |
| 28 | P1 Stem- | Introduce C:G, | Increase | (N20)GTCCATAACCCGAAAGGGTTACGGAGC |
| | Loop | Position 2 | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 369) |
| 29 | P1 Stem- | Introduce G:C, | Increase | (N20)GTCAAGAACCCGAAAGGGTTCCTGAGC |
| | Loop | Position 5 | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 370) |
| 30 | P1 Stem- | Introduce C:G, | Increase | (N20)GTCAACAACCCGAAAGGGTTGCTGAGC |
| | Loop | Position 5 | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 371) |
| 31 | P1 Stem- | Introduce G:C, | Increase | (N20)GTCAATGACCCGAAAGGGTCACTGAGC |
| | Loop | Position 6 | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 372) |
| 32 | P1 Stem- | Introduce C:G, | Increase | (N20)GTCAATCACCCGAAAGGGTGACTGAGC |
| | Loop | Position 6 | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 373) |
| 33 | P1 Stem- | Introduce G:C, | Increase | (N20)GTCAATAGCCCGAAAGGGCTACTGAGC |
| | Loop | Position 7 | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 374) |
| 34 | P1 Stem- | Introduce C:G, | Increase | (N20)GTCAATACCCCGAAAGGGGTACTGAGC |
| | Loop | Position 7 | stability of | AGAGCAGTGACATGTCATTCGCCGCGGGGTG |
| | | | P1 stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 375) |
| 35 | P1 Stem- | Delete U:A | Reduce | (N20)GTCAATACCCGAAAGGGTACTGAGCAG |
| | Loop | Position 7 | length | AGCAGTGACATGTCATTCGCCGCGGGGTGAT |
| | | | | TCCAAGCTCCGCGCTCCGGCTAGACATGCCC |
| | | | | ATGCTATGGAAACTTTAACGGTGAAAACCGG |
| | | | | CTTACAACAAA (SEQ ID NO: 376) |
| 36 | P1 Stem- | Delete U:A | Reduce | (N20)GTCAATCCCGAAAGGGACTGAGCAGAG |
| | Loop | Position 6-7 | length | CAGTGACATGTCATTCGCCGCGGGGTGATTC |
| | | | | CAAGCTCCGCGCTCCGGCTAGACATGCCCAT |
| | | | | GCTATGGAAACTTTAACGGTGAAAACCGGCT |
| | | | | TACAACAAA (SEQ ID NO: 377) |
| 37 | P1 Stem- | Delete U:A | Reduce | (N20)GTCAACCCGAAAGGGCTGAGCAGAGCA |
| | Loop | Position 6-8 | length | GTGACATGTCATTCGCCGCGGGGTGATTCCA |
| | | | | AGCTCCGCGCTCCGGCTAGACATGCCCATGC |
| | | | | TATGGAAACTTTAACGGTGAAAACCGGCTTA |
| | | | | CAACAAA (SEQ ID NO: 378) |
| 38 | 3′-end | Del 1 | Reduce | (N20)GTCAATAACCCGAAAGGGTTACTGAGC |
| | | | length | AGAGCAGTGACATGTCATTCGCCGCGGGGTG |
| | | | | ATTCCAAGCTCCGCGCTCCGGCTAGACATGC |
| | | | | CCATGCTATGGAAACTTTAACGGTGAAAACC |
| | | | | GGCTTACAACAA (SEQ ID NO: 379) |
| 39 | 3′-end | Del 2 | Reduce | (N20)GTCAATAACCCGAAAGGGTTACTGAGC |
| | | | length | AGAGCAGTGACATGTCATTCGCCGCGGGGTG |
| | | | | ATTCCAAGCTCCGCGCTCCGGCTAGACATGC |
| | | | | CCATGCTATGGAAACTTTAACGGTGAAAACC |
| | | | | GGCTTACAACA (SEQ ID NO: 380) |
| 40 | 3′-end | Del 3 | Reduce | (N20)GTCAATAACCCGAAAGGGTTACTGAGC |
| | | | length | AGAGCAGTGACATGTCATTCGCCGCGGGGTG |
| | | | | ATTCCAAGCTCCGCGCTCCGGCTAGACATGC |
| | | | | CCATGCTATGGAAACTTTAACGGTGAAAACC |
| | | | | GGCTTACAAC (SEQ ID NO: 381) |
| 41 | 3′-end | Del 4 | Reduce | (N20)GTCAATAACCCGAAAGGGTTACTGAGC |
| | | | length | AGAGCAGTGACATGTCATTCGCCGCGGGGTG |
| | | | | ATTCCAAGCTCCGCGCTCCGGCTAGACATGC |
| | | | | CCATGCTATGGAAACTTTAACGGTGAAAACC |
| | | | | GGCTTACAA (SEQ ID NO: 382) |
| 42 | 3′-end | Del 5 | Reduce | (N20)GTCAATAACCCGAAAGGGTTACTGAGC |
| | | | length | AGAGCAGTGACATGTCATTCGCCGCGGGGTG |
| | | | | ATTCCAAGCTCCGCGCTCCGGCTAGACATGC |
| | | | | CCATGCTATGGAAACTTTAACGGTGAAAACC |
| | | | | GGCTTACA (SEQ ID NO: 383) |
| 43 | 3′-end | Del 6 | Reduce | (N20)GTCAATAACCCGAAAGGGTTACTGAGC |
| | | | length | AGAGCAGTGACATGTCATTCGCCGCGGGGTG |
| | | | | ATTCCAAGCTCCGCGCTCCGGCTAGACATGC |
| | | | | CCATGCTATGGAAACTTTAACGGTGAAAACC |
| | | | | GGCTTAC (SEQ ID NO: 343) |
| 44 | 3′-end | Del 7 | Reduce | (N20)GTCAATAACCCGAAAGGGTTACTGAGC |
| | | | length | AGAGCAGTGACATGTCATTCGCCGCGGGGTG |
| | | | | ATTCCAAGCTCCGCGCTCCGGCTAGACATGC |
| | | | | CCATGCTATGGAAACTTTAACGGTGAAAACC |
| | | | | GGCTTA (SEQ ID NO: 342) |
| 45 | 3′-end | Del 8 | Reduce | (N20)GTCAATAACCCGAAAGGGTTACTGAGC |
| | | | length | AGAGCAGTGACATGTCATTCGCCGCGGGGTG |
| | | | | ATTCCAAGCTCCGCGCTCCGGCTAGACATGC |
| | | | | CCATGCTATGGAAACTTTAACGGTGAAAACC |
| | | | | GGCTT (SEQ ID NO: 384) |
| 46 | 3′-end | Del 9 | Reduce | (N20)GTCAATAACCCGAAAGGGTTACTGAGC |
| | | | length | AGAGCAGTGACATGTCATTCGCCGCGGGGTG |
| | | | | ATTCCAAGCTCCGCGCTCCGGCTAGACATGC |
| | | | | CCATGCTATGGAAACTTTAACGGTGAAAACC |
| | | | | GGCT (SEQ ID NO: 385) |
| 47 | 3′-end | Del 10 | Reduce | (N20)GTCAATAACCCGAAAGGGTTACTGAGC |
| | | | length | AGAGCAGTGACATGTCATTCGCCGCGGGGTG |
| | | | | ATTCCAAGCTCCGCGCTCCGGCTAGACATGC |
| | | | | CCATGCTATGGAAACTTTAACGGTGAAAACC |
| | | | | GGC (SEQ ID NO: 386) |
| 48 | 3′-end | Del 11 | Reduce | (N20)GTCAATAACCCGAAAGGGTTACTGAGC |
| | | | length | AGAGCAGTGACATGTCATTCGCCGCGGGGTG |
| | | | | ATTCCAAGCTCCGCGCTCCGGCTAGACATGC |
| | | | | CCATGCTATGGAAACTTTAACGGTGAAAACC |
| | | | | GG (SEQ ID NO: 387) |
| 49 | G:U | G55:U68 to | Increase | (N20)GTCAATAACCCGAAAGGGTTACTGAGC |
| | | G55:C68 | stability of | AGAGCAGTGACATGTCATTCGCCGCGGGGTG |
| | | | P3 Stem | ATTCCAAGCCCCGCGCTCCGGCTAGACATGC |
| | | | | CCATGCTATGGAAACTTTAACGGTGAAAACC |
| | | | | GGCTTACAACAAA (SEQ ID NO: 388) |
| 50 | G:U | G55:U68 to | Increase | (N20)GTCAATAACCCGAAAGGGTTACTGAGC |
| | | C55:G68 | stability of | AGAGCAGTGACATGTCATTCGCCGCGGCGTG |
| | | | P3 Stem | ATTCCAAGCGCCGCGCTCCGGCTAGACATGC |
| | | | | CCATGCTATGGAAACTTTAACGGTGAAAACC |
| | | | | GGCTTACAACAAA (SEQ ID NO: 389) |
| 51 | G:U | G55:U68 to | Increase | (N20)GTCAATAACCCGAAAGGGTTACTGAGC |
| | | A55:U68 | stability of | AGAGCAGTGACATGTCATTCGCCGCGGAGTG |
| | | | P3 Stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 390) |
| 52 | G:U | U57:G66 to | Increase | (N20)GTCAATAACCCGAAAGGGTTACTGAGC |
| | | C57:G66 | stability of | AGAGCAGTGACATGTCATTCGCCGCGGGGCG |
| | | | P3 Stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 391) |
| 53 | G:U | U57:G66 to | Increase | (N20)GTCAATAACCCGAAAGGGTTACTGAGC |
| | | G57:C66 | stability of | AGAGCAGTGACATGTCATTCGCCGCGGGGGG |
| | | | P3 Stem | ATTCCAACCTCCGCGCTCCGGCTAGACATGC |
| | | | | CCATGCTATGGAAACTTTAACGGTGAAAACC |
| | | | | GGCTTACAACAAA (SEQ ID NO: 392) |
| 54 | G:U | U57:G66 to | Increase | (N20)GTCAATAACCCGAAAGGGTTACTGAGC |
| | | U57:A66 | stability of | AGAGCAGTGACATGTCATTCGCCGCGGGGTG |
| | | | P3 Stem | ATTCCAAACTCCGCGCTCCGGCTAGACATGC |
| | | | | CCATGCTATGGAAACTTTAACGGTGAAAACC |
| | | | | GGCTTACAACAAA (SEQ ID NO: 393) |
| 55 | G:U | U46:G79 to | Increase | (N20)GTCAATAACCCGAAAGGGTTACTGAGC |
| | | C46:G79 | stability of | AGAGCAGTGACATGTCATCCGCCGCGGGGTG |
| | | | P3 Stem | ATTCCAAGCTCCGCGCTCCGGCTAGACATGC |
| | | | | CCATGCTATGGAAACTTTAACGGTGAAAACC |
| | | | | GGCTTACAACAAA (SEQ ID NO: 394) |
| 56 | G:U | U46:G79 to | Increase | (N20)GTCAATAACCCGAAAGGGTTACTGAGC |
| | | G46:C79 | stability of | AGAGCAGTGACATGTCATGCGCCGCGGGGTG |
| | | | P3 Stem | ATTCCAAGCTCCGCGCTCCGCCTAGACATGC |
| | | | | CCATGCTATGGAAACTTTAACGGTGAAAACC |
| | | | | GGCTTACAACAAA (SEQ ID NO: 395) |
| 57 | G:U | U46:G79 to | Increase | (N20)GTCAATAACCCGAAAGGGTTACTGAGC |
| | | U46:A79 | stability of | AGAGCAGTGACATGTCATTCGCCGCGGGGTG |
| | | | P3 Stem | ATTCCAAGCTCCGCGCTCCGACTAGACATGC |
| | | | | CCATGCTATGGAAACTTTAACGGTGAAAACC |
| | | | | GGCTTACAACAAA (SEQ ID NO: 396) |
| 58 | Pseudo- | U60:A102 to | Modulate | (N20)GTCAATAACCCGAAAGGGTTACTGAGC |
| | knot | A60:A102 | folding | AGAGCAGTGACATGTCATTCGCCGCGGGGTG |
| | | | stability of | AATCCAAGCTCCGCGCTCCGGCTAGACATGC |
| | | | Pseudoknot | CCATGCTATGGAAACTTTAACGGTGAAAACC |
| | | | | GGCTTACAACAAA (SEQ ID NO: 397) |
| 59 | Pseudo- | U60:A102 to | Modulate | (N20)GTCAATAACCCGAAAGGGTTACTGAGC |
| | knot | C60:G102 | folding | AGAGCAGTGACATGTCATTCGCCGCGGGGTG |
| | | | stability of | ACTCCAAGCTCCGCGCTCCGGCTAGACATGC |
| | | | Pseudoknot | CCATGCTATGGAGACTTTAACGGTGAAAACC |
| | | | | GGCTTACAACAAA (SEQ ID NO: 398) |
| 60 | Pseudo- | U60:A102 to | Modulate | (N20)GTCAATAACCCGAAAGGGTTACTGAGC |
| | knot | G60:C102 | folding | AGAGCAGTGACATGTCATTCGCCGCGGGGTG |
| | | | stability of | AGTCCAAGCTCCGCGCTCCGGCTAGACATGC |
| | | | Pseudoknot | CCATGCTATGGACACTTTAACGGTGAAAACC |
| | | | | GGCTTACAACAAA (SEQ ID NO: 399) |
| 61 | Pseudo- | U61:A101 to | Modulate | (N20)GTCAATAACCCGAAAGGGTTACTGAGC |
| | knot | A61:A101 | folding | AGAGCAGTGACATGTCATTCGCCGCGGGGTG |
| | | | stability of | ATACCAAGCTCCGCGCTCCGGCTAGACATGC |
| | | | Pseudoknot | CCATGCTATGGAAACTTTAACGGTGAAAACC |
| | | | | GGCTTACAACAAA (SEQ ID NO: 400) |
| 62 | Pseudo- | U61:A101 to | Modulate | (N20)GTCAATAACCCGAAAGGGTTACTGAGC |
| | knot | C61:G101 | folding | AGAGCAGTGACATGTCATTCGCCGCGGGGTG |
| | | | stability of | ATCCCAAGCTCCGCGCTCCGGCTAGACATGC |
| | | | Pseudoknot | CCATGCTATGGGAACTTTAACGGTGAAAACC |
| | | | | GGCTTACAACAAA (SEQ ID NO: 401) |
| 63 | Pseudo- | U61:A101 to | Modulate | (N20)GTCAATAACCCGAAAGGGTTACTGAGC |
| | knot | G61:C101 | folding | AGAGCAGTGACATGTCATTCGCCGCGGGGTG |
| | | | stability of | ATGCCAAGCTCCGCGCTCCGGCTAGACATGC |
| | | | Pseudoknot | CCATGCTATGGCAACTTTAACGGTGAAAACC |
| | | | | GGCTTACAACAAA (SEQ ID NO: 402) |
| 64 | Pseudo- | U60:A102/U61: | Modulate | (N20)GTCAATAACCCGAAAGGGTTACTGAGC |
| | knot | A101 to | folding | AGAGCAGTGACATGTCATTCGCCGCGGGGTG |
| | | C60:G102/C61: | stability of | ACCCCAAGCTCCGCGCTCCGGCTAGACATGC |
| | | G101 | Pseudoknot | CCATGCTATGGGGACTTTAACGGTGAAAACC |
| | | | | GGCTTACAACAAA (SEQ ID NO: 403) |
| 65 | Pseudo- | U60:A102/U61: | Modulate | (N20)GTCAATAACCCGAAAGGGTTACTGAGC |
| | knot | A101 to | folding | AGAGCAGTGACATGTCATTCGCCGCGGGGTG |
| | | G60:C102/C61: | stability of | AGCCCAAGCTCCGCGCTCCGGCTAGACATGC |
| | | G101 | Pseudoknot | CCATGCTATGGGCACTTTAACGGTGAAAACC |
| | | | | GGCTTACAACAAA (SEQ ID NO: 404) |
| 66 | Pseudo- | U60:A102/U61: | Modulate | (N20)GTCAATAACCCGAAAGGGTTACTGAGC |
| | knot | A101 to | folding | AGAGCAGTGACATGTCATTCGCCGCGGGGTG |
| | | C60:G102/G61: | stability of | ACGCCAAGCTCCGCGCTCCGGCTAGACATGC |
| | | C101 | Pseudoknot | CCATGCTATGGCGACTTTAACGGTGAAAACC |
| | | | | GGCTTACAACAAA (SEQ ID NO: 405) |
| 67 | Pseudo- | U60:A102/U61: | Modulate | (N20)GTCAATAACCCGAAAGGGTTACTGAGC |
| | knot | A101 to | folding | AGAGCAGTGACATGTCATTCGCCGCGGGGTG |
| | | G60:C102/G61: | stability of | AGGCCAAGCTCCGCGCTCCGGCTAGACATGC |
| | | C101 | Pseudoknot | CCATGCTATGGCCACTTTAACGGTGAAAACC |
| | | | | GGCTTACAACAAA (SEQ ID NO: 406) |
| 68 | Pseudo- | A64:U98 to | Modulate | (N20)GTCAATAACCCGAAAGGGTTACTGAGC |
| | knot | U64:U98 | folding | AGAGCAGTGACATGTCATTCGCCGCGGGGTG |
| | | | stability of | ATTCCTAGCTCCGCGCTCCGGCTAGACATGC |
| | | | Pseudoknot | CCATGCTATGGAAACTTTAACGGTGAAAACC |
| | | | | GGCTTACAACAAA (SEQ ID NO: 407) |
| 69 | Pseudo- | A64:U98 to | Modulate | (N20)GTCAATAACCCGAAAGGGTTACTGAGC |
| | knot | C64:G98 | folding | AGAGCAGTGACATGTCATTCGCCGCGGGGTG |
| | | | stability of | ATTCCCAGCTCCGCGCTCCGGCTAGACATGC |
| | | | Pseudoknot | CCATGCTAGGGAAACTTTAACGGTGAAAACC |
| | | | | GGCTTACAACAAA (SEQ ID NO: 408) |
| 70 | Pseudo- | A64:U98 to | Modulate | (N20)GTCAATAACCCGAAAGGGTTACTGAGC |
| | knot | G64:C98 | folding | AGAGCAGTGACATGTCATTCGCCGCGGGGTG |
| | | | stability of | ATTCCGAGCTCCGCGCTCCGGCTAGACATGC |
| | | | Pseudoknot | CCATGCTACGGAAACTTTAACGGTGAAAACC |
| | | | | GGCTTACAACAAA (SEQ ID NO: 409) |
| 71 | Pseudo- | Ins60U | Modulate | (N20)GTCAATAACCCGAAAGGGTTACTGAGC |
| | knot | | folding | AGAGCAGTGACATGTCATTCGCCGCGGGGTG |
| | | | stability of | ATTTCCAAGCTCCGCGCTCCGGCTAGACATG |
| | | | Pseudoknot | CCCATGCTATGGAAACTTTAACGGTGAAAAC |
| | | | | CGGCTTACAACAAA (SEQ ID NO: 410) |
| 72 | Pseudo- | Ins60CU | Modulate | (N20)GTCAATAACCCGAAAGGGTTACTGAGC |
| | knot | | folding | AGAGCAGTGACATGTCATTCGCCGCGGGGTG |
| | | | stability of | ACTTTCCAAGCTCCGCGCTCCGGCTAGACAT |
| | | | Pseudoknot | GCCCATGCTATGGAAACTTTAACGGTGAAAA |
| | | | | CCGGCTTACAACAAA (SEQ ID NO: 411) |
| 73 | Pseudo- | Ins60AU | Modulate | (N20)GTCAATAACCCGAAAGGGTTACTGAGC |
| | knot | | folding | AGAGCAGTGACATGTCATTCGCCGCGGGGTG |
| | | | stability of | AATTTCCAAGCTCCGCGCTCCGGCTAGACAT |
| | | | Pseudoknot | GCCCATGCTATGGAAACTTTAACGGTGAAAA |
| | | | | CCGGCTTACAACAAA (SEQ ID NO: 412) |
| 74 | Pseudo- | Ins60CCU | Modulate | (N20)GTCAATAACCCGAAAGGGTTACTGAGC |
| | knot | | folding | AGAGCAGTGACATGTCATTCGCCGCGGGGTG |
| | | | stability of | ACCTTTCCAAGCTCCGCGCTCCGGCTAGACA |
| | | | Pseudoknot | TGCCCATGCTATGGAAACTTTAACGGTGAAA |
| | | | | ACCGGCTTACAACAAA (SEQ ID NO: 413) |
| 75 | Pseudo- | Ins60TAU | Modulate | (N20)GTCAATAACCCGAAAGGGTTACTGAGC |
| | knot | | folding | AGAGCAGTGACATGTCATTCGCCGCGGGGTG |
| | | | stability of | ATATTTCCAAGCTCCGCGCTCCGGCTAGACA |
| | | | Pseudoknot | TGCCCATGCTATGGAAACTTTAACGGTGAAA |
| | | | | ACCGGCTTACAACAAA (SEQ ID NO: 414) |
| 76 | Pseudo- | Ins60GU | Modulate | (N20)GTCAATAACCCGAAAGGGTTACTGAGC |
| | knot | | folding | AGAGCAGTGACATGTCATTCGCCGCGGGGTG |
| | | | stability of | AGTTTCCAAGCTCCGCGCTCCGGCTAGACAT |
| | | | Pseudoknot | GCCCATGCTATGGAAACTTTAACGGTGAAAA |
| | | | | CCGGCTTACAACAAA (SEQ ID NO: 415) |
| 77 | Pseudo- | Ins60AGU | Modulate | (N20)GTCAATAACCCGAAAGGGTTACTGAGC |
| | knot | | folding | AGAGCAGTGACATGTCATTCGCCGCGGGGTG |
| | | | stability of | AAGTTTCCAAGCTCCGCGCTCCGGCTAGACA |
| | | | Pseudoknot | TGCCCATGCTATGGAAACTTTAACGGTGAAA |
| | | | | ACCGGCTTACAACAAA (SEQ ID NO: 416) |
| 78 | Pseudo- | Ins60CGU | Modulate | (N20)GTCAATAACCCGAAAGGGTTACTGAGC |
| | knot | | folding | AGAGCAGTGACATGTCATTCGCCGCGGGGTG |
| | | | stability of | ACGTTTCCAAGCTCCGCGCTCCGGCTAGACA |
| | | | Pseudoknot | TGCCCATGCTATGGAAACTTTAACGGTGAAA |
| | | | | ACCGGCTTACAACAAA (SEQ ID NO: 417) |
| 79 | Pseudo- | Ins60TGU | Modulate | (N20)GTCAATAACCCGAAAGGGTTACTGAGC |
| | knot | | folding | AGAGCAGTGACATGTCATTCGCCGCGGGGTG |
| | | | stability of | ATGTTTCCAAGCTCCGCGCTCCGGCTAGACA |
| | | | Pseudoknot | TGCCCATGCTATGGAAACTTTAACGGTGAAA |
| | | | | ACCGGCTTACAACAAA (SEQ ID NO: 418) |
| 80 | Pseudo- | Ins60GGU | Modulate | (N20)GTCAATAACCCGAAAGGGTTACTGAGC |
| | knot | | folding | AGAGCAGTGACATGTCATTCGCCGCGGGGTG |
| | | | stability of | AGGTTTCCAAGCTCCGCGCTCCGGCTAGACA |
| | | | Pseudoknot | TGCCCATGCTATGGAAACTTTAACGGTGAAA |
| | | | | ACCGGCTTACAACAAA (SEQ ID NO: 419) |
| 81 | Pseudo- | Ins60AAGU | Modulate | (N20)GTCAATAACCCGAAAGGGTTACTGAGC |
| | knot | | folding | AGAGCAGTGACATGTCATTCGCCGCGGGGTG |
| | | | stability of | AAAGTTTCCAAGCTCCGCGCTCCGGCTAGAC |
| | | | Pseudoknot | ATGCCCATGCTATGGAAACTTTAACGGTGAA |
| | | | | AACCGGCTTACAACAAA (SEQ ID NO: 420) |
| 82 | Pseudo- | Ins60GAGU | Modulate | (N20)GTCAATAACCCGAAAGGGTTACTGAGC |
| | knot | | folding | AGAGCAGTGACATGTCATTCGCCGCGGGGTG |
| | | | stability of | AGAGTTTCCAAGCTCCGCGCTCCGGCTAGAC |
| | | | Pseudoknot | ATGCCCATGCTATGGAAACTTTAACGGTGAA |
| | | | | AACCGGCTTACAACAAA (SEQ ID NO: 421) |
| 83 | Pseudo- | Ins60AGGU | Modulate | (N20)GTCAATAACCCGAAAGGGTTACTGAGC |
| | knot | | folding | AGAGCAGTGACATGTCATTCGCCGCGGGGTG |
| | | | stability of | AAGGTTTCCAAGCTCCGCGCTCCGGCTAGAC |
| | | | Pseudoknot | ATGCCCATGCTATGGAAACTTTAACGGTGAA |
| | | | | AACCGGCTTACAACAAA (SEQ ID NO: 422) |
| 84 | Pseudo- | Ins60CTGU | Modulate | (N20)GTCAATAACCCGAAAGGGTTACTGAGC |
| | knot | | folding | AGAGCAGTGACATGTCATTCGCCGCGGGGTG |
| | | | stability of | ACTGTTTCCAAGCTCCGCGCTCCGGCTAGAC |
| | | | Pseudoknot | ATGCCCATGCTATGGAAACTTTAACGGTGAA |
| | | | | AACCGGCTTACAACAAA (SEQ ID NO: 423) |
| 85 | Pseudo- | Ins60TCGU | Modulate | (N20)GTCAATAACCCGAAAGGGTTACTGAGC |
| | knot | | folding | AGAGCAGTGACATGTCATTCGCCGCGGGGTG |
| | | | stability of | ATCGTTTCCAAGCTCCGCGCTCCGGCTAGAC |
| | | | Pseudoknot | ATGCCCATGCTATGGAAACTTTAACGGTGAA |
| | | | | AACCGGCTTACAACAAA (SEQ ID NO: 424) |
| 86 | Pseudo- | Ins60AAAGU | Modulate | (N20)GTCAATAACCCGAAAGGGTTACTGAGC |
| | knot | | folding | AGAGCAGTGACATGTCATTCGCCGCGGGGTG |
| | | | stability of | AAAAGTTTCCAAGCTCCGCGCTCCGGCTAGA |
| | | | Pseudoknot | CATGCCCATGCTATGGAAACTTTAACGGTGA |
| | | | | AAACCGGCTTACAACAAA (SEQ ID NO: 425) |
| 87 | Pseudo- | Ins60GAAAGU | Modulate | (N20)GTCAATAACCCGAAAGGGTTACTGAGC |
| | knot | | folding | AGAGCAGTGACATGTCATTCGCCGCGGGGTG |
| | | | stability of | AGAAAGTTTCCAAGCTCCGCGCTCCGGCTAG |
| | | | Pseudoknot | ACATGCCCATGCTATGGAAACTTTAACGGTG |
| | | | | AAAACCGGCTTACAACAAA (SEQ ID NO: 426) |
| 88 | Pseudo- | Ins60TAGAAA | Modulate | (N20)GTCAATAACCCGAAAGGGTTACTGAGC |
| | knot | GU | folding | AGAGCAGTGACATGTCATTCGCCGCGGGGTG |
| | | | stability of | ATAGAAAGTTTCCAAGCTCCGCGCTCCGGCT |
| | | | Pseudoknot | AGACATGCCCATGCTATGGAAACTTTAACGG |
| | | | | TGAAAACCGGCTTACAACAAA (SEQ ID NO: |
| | | | | 427) |
| 89 | Pseudo- | Ins60ATGAAA | Modulate | (N20)GTCAATAACCCGAAAGGGTTACTGAGC |
| | knot | GU | folding | AGAGCAGTGACATGTCATTCGCCGCGGGGTG |
| | | | stability of | AATGAAAGTTTCCAAGCTCCGCGCTCCGGCT |
| | | | Pseudoknot | AGACATGCCCATGCTATGGAAACTTTAACGG |
| | | | | TGAAAACCGGCTTACAACAAA (SEQ ID NO: |
| | | | | 428) |
| 90 | Pseudo- | Ins60ACGAAA | Modulate | (N20)GTCAATAACCCGAAAGGGTTACTGAGC |
| | knot | GU | folding | AGAGCAGTGACATGTCATTCGCCGCGGGGTG |
| | | | stability of | AACGAAAGTTTCCAAGCTCCGCGCTCCGGCT |
| | | | Pseudoknot | AGACATGCCCATGCTATGGAAACTTTAACGG |
| | | | | TGAAAACCGGCTTACAACAAA (SEQ ID NO: |
| | | | | 429) |
| 91 | Pseudo- | Ins60CAGAAA | Modulate | (N20)GTCAATAACCCGAAAGGGTTACTGAGC |
| | knot | GU | folding | AGAGCAGTGACATGTCATTCGCCGCGGGGTG |
| | | | stability of | ACAGAAAGTTTCCAAGCTCCGCGCTCCGGCT |
| | | | Pseudoknot | AGACATGCCCATGCTATGGAAACTTTAACGG |
| | | | | TGAAAACCGGCTTACAACAAA (SEQ ID NO: |
| | | | | 430) |
| 92 | Pseudo- | Ins60TGTGAAAAGU | Modulate | (N20)GTCAATAACCCGAAAGGGTTACTGAGC |
| | knot | (“TGTGAAAAGU” | folding | AGAGCAGTGACATGTCATTCGCCGCGGGGTG |
| | | disclosed | stability of | ATGTGAAAAGTTTCCAAGCTCCGCGCTCCGG |
| | | as SEQ ID NO: | Pseudoknot | CTAGACATGCCCATGCTATGGAAACTTTAAC |
| | | 439) | | GGTGAAAACCGGCTTACAACAAA (SEQ ID |
| | | | | NO: 431) |

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. An IscB protein (protein effector polypeptide) comprising:
(a) an amino acid sequence that is at least 80% identical to the amino acid sequence of an IscB as provided by any one of SEQ ID NO: 2, wherein the amino acid sequence of the IscB protein comprises at least one, at least two, at least three, at least four, at least five, at least six, at least seven, at least eight, or at least nine mutations in an amino acid residue selected from the group consisting of amino acid residues 18, 34, 36, 37, 60, 66, 85, 97, 99, 100, 101, 104, 109, 112, 118, 136, 137, 155, 156, 157, 166, 168, 190, 204, 204, 205, 206, 208, 212, 214, 216, 218, 219, 229, 229, 230, 233, 233, 234, 234, 238, 239, 241, 242, 245, 246, 248, 252, 254, 255, 256, 257, 258, 279, 280, 291, 293, 298, 303, 304, 306, 307, 312, 319, 323, 328, 329, 331, 332, 338, 341, 353, 360, 368, 372, 376, 377, 378, 379, 380, 381, 382, 384, 385, 386, 388, 390, 391, 393, 405, 406, 408, 409, 410, 412, 413, 416, 417, 418, 419, 421, 421, 423, 425, 437, 439, 455, 462, 463, 478, 479, 533, 536, 564 and 565 of the amino acid sequence of SEQ ID NO: 2 (pEDIT071), and provided that, if the IscB comprises a single mutation, it is not Q60I, N66H, I85R, I85K, E137R, P405R, D413K, E409R or combination thereof; or
(b) an amino acid sequence that is at least 80% identical to the amino acid sequence of an IscB as provided by any one of SEQ ID NO: 8, wherein the amino acid sequence of the IscB protein comprises at least one, at least two, at least three, at least four, at least five, at least six, at least seven, at least eight, or at least nine mutations in an amino acid residue selected from the group consisting of amino acid residues 30, 46, 48, 49, 72, 78, 97, 109, 111, 112, 113, 116, 121, 124, 130, 148, 149, 167, 168, 169, 178, 180, 202, 216, 216, 217, 218, 220, 224, 226, 228, 230, 231, 241, 241, 242, 245, 245, 246, 246, 250, 251, 253, 254, 257, 258, 260, 264, 266, 267, 268, 269, 270, 291, 292, 303, 305, 310, 315, 316, 318, 319, 324, 331, 335, 340, 341, 343, 344, 350, 353, 365, 372, 380, 384, 388, 389, 390, 391, 392, 393, 394, 396, 397, 398, 400, 402, 403, 405, 417, 418, 420, 421, 422, 424, 425, 428, 429, 430, 431, 433, 433, 435, 437, 449, 451, 467, 474, 475, 490, 491, 545, 548, 576, and 577 of the amino acid sequence of SEQ ID NO: 8 (pEDIT071+NLS), and provided that, if the IscB comprises a single mutation, and optionally, if the IscB comprises a single mutation, the mutation is not Q72I, H78H, I97R, 97K, E149R, P417R, E421R, D425K, or combination thereof.

2. The IscB protein of claim 1, wherein the amino acid sequence of the IscB protein comprises:
(a) at least one, at least two, at least three, at least four, at least five, at least six, at least seven, at least eight, or at least nine mutations selected from the group consisting of variant C18R, A34R, D36R, T37R, Q60I, N66H, I85X, 185X, M99X, A101X, A104R, E109X, A112K, R118K, S136X, E137R, I155X, L156X, T157X, T166X, L168K, L190K, G204X, N205X, I206K, K208R, K212R, K214R, N216R, K218R, D219X, A229X, K230R, L233X, P234X, P238X, F239X, K241R, E242X, A245X, E246X, E248R, T252X, E254X, K255R, S256X, T257X, F258X, S279R, G280R, L291X, K293R, Q298K, E303X, M304K, K306R, R307K, C312X, T319K, E323X, E328R, Q329R, D331R, G332R, G338R, S341R, R353K, A360X, K368R, L372R, D376R, A377R, D378R, T379R, A380R, E381R, S382R, T384R, E385R, M386R, T388R, L390X, M391X, K393R, P405K, K406R, V408R, E409X, T410R, A412R, D413R, P416R, G417R, H418K, F419R, V421X, N423R, W425K, E437R, D439X, E455R, E462R, P463R, I533K, T536R, H478X, H479X, I564X, and T565X of the amino acid sequence provided in SEQ ID NO: 2, wherein X is any amino acid, and optionally, if the IscB comprises a single mutation, it is not Q60I, N66H, I85R, 185K, E137R, P405R, D413K, E409R or combination thereof; or
(b) at least one, at least two, at least three, at least four, at least five, at least six, at least seven, at least eight, or at least nine mutations selected independently from the group consisting of variant C30R, A46R, D48R, T49R, Q72I, N78H, I97X, I109X, M111X, A1113X, A116R, E121X, A124K, R130K, S148X, E149R, I167X, L168X, T169X, T178X, L180K, L202K, G216X, N217X, I218K, K2220R, K224R, K2226R, N228R, K230R, D231X, A241X, K242R, L245X, P246X, P250X, F251X, K253R, E254X, A257X, E258X, E260R, T264X, E266X, K267R, S268X, T269X, F270X, S291R, G292R, L303X, K2305R, Q310K, E315X, M316K, K318R, R319K, C324X, T331K, E335X, E340R, Q343R, G344R, G350R, S353R, R365K, A372X, K380R, L384R, D388R, A389R, D390R, T391R, A392R, E393R, S394R, T396R, E397R, M398R, T400R, L402X, M403X, K3405R, P417K, K418R, V420R, E421X, T422R, A424R, D425R, P428R, G429R, H430K, F431R, V433X, N435R, W437K, E449R, D451X, E467R, E474R, P475R, I545K, T548R, H490X, H491X, I576X, and T577X of the amino acid sequence SEQ ID NO: 8, wherein X is any amino acid, and optionally, if the IscB comprises a single mutation, the mutation is not Q72I, H78H, I97R, I97K, E149R, P417R, E421R, D425K, or combination thereof.

3. The IscB protein of claim 1, wherein the amino acid sequence of the IscB protein comprises:
(a) at least one, at least two, at least three, at least four, at least five, at least six, at least seven, at least eight, or at least nine mutations selected from the group consisting of variant C18R, A34R, D36R, T37R, Q60I, N66H, I85X, 185X, M99X, A101X, A104R, E109X, A112K, R118K, S136X, E137R, I155X, L156X, T157X, T166X, L168K, L190K, G204X, N205X, I206K, K208R, K212R, K214R, N216R, K218R, D219X, A229X, K230R, L233X, P234X, P238X, F239X, K241R, E242X, A245X, E246X, E248R, T252X, E254X, K255R, S256X, T257X, F258X, S279R, G280R, L291X, K293R, Q298K, E303X, M304K, K306R, R307K, C312X, T319K, E323X, E328R, Q329R, D331R, G332R, G338R, S341R, R353K, A360X, K368R, L372R, D376R, A377R, D378R, T379R, A380R, E381R, S382R, T384R, E385R, M386R, T388R, L390X, M391X, K393R, P405K, K406R, V408R, E409X, T410R, A412R, D413R, P416R, G417R, H418K, F419R, V421X, N423R, W425K, E437R, D439X, E455R, E462R, P463R, I533K, T536R, H478N, H479N, I564K, I564H, I564N, I564Q, I564C, I564M, I564T, I564D, T565R, T565K, T565H, T565C, T565D, T565G, H478C, H478P, H479C, and H479M of the amino acid sequence provided in SEQ ID NO: 2, and optionally, if the IscB comprises a single mutation, it is not Q60I, N66H, I85R, I85K, E137R, P405R, D413K, E409R or combination thereof; or
(b) at least one, at least two, at least three, at least four, at least five, at least six, at least seven, at least eight, or at least nine mutations selected independently from the group consisting of C30R, A46R, D46R, T49R, Q72I, N78H, I97R, I97K, D109R, D109K, M111R, M111K, A112K, A113R, A113K, A116R, E121R, E121K, A124K, R130K, S148R, S148K, E149R, E149K, 1167R, 1167K, L168R, L168K, T169R, T169K, T178R, T178K, L180K, L1202K, G216R, G216K, N217R, N217K, I218K, K220R, K224R, K2226R, N228R, K230R, D231R, D231K, A241R, A241K, K242R, L245R, L245K, P246R, P246K, P250R, P250K, F251R, F251K, K253R, E254R, E2454K, A257R, A257K, E258R, E2458K, E260R, T264R, T264K, E266R, E266K, K267R, S268R, S268K, T2569R, T2569K, F270R, F270K, S291R, G292R, L303R, L303K, K305R, Q310K, E315R, E315K, M316K, K318R, R319K, C324R, C324K, T331K, E335R, E335K, E340R, Q341R, D343R, G344R, G350R, S353R, R365K, A372R, A372K, K380R, L384R, D388R, A389R, D390R, T391R, A392R, E393R, S394R, T396R, E397R, M398R, T400R, L402R, L402K, M4031R, M403K, K405R, P417K, K419R, V420R, E421K, E421R, T422R, A424R, D425R, D425K, P428R, G429R, H430K, F431R, V433R, V433K, N435R, W437K, E449R, D451R, D451K, E4567R, E474R, P475R, I545K, T548R, H490N, H491N, I576K, I576H, I576N, I576Q, I576C, I576M, I576T, I5676D, T577R, T577K, T577H, T577C, T577D, T577G, H490C, H490P, H491C, and H491M of the amino acid sequence provided in SEQ ID NO: 8. Optionally, if the IscB comprises a single mutation, the mutation is not Q72I, H78H, 197R, I97K, E149R, P417R, E421R, D425K, or combination thereof.

4. The IscB protein of any one of claims 1-3, wherein the IscB protein comprises: (i) at least E409R, D413K, 533K, and at least one other mutation listed in Table A; or (ii) at least E421R, D425K, 545K, and at least one other mutation listed in Table A2.

5. The IscB protein of any one of claims 1-3, wherein the IscB protein comprises: (i) at least E409R, D413K, 533K E137K, N66H, A100K, T536R, and at least one other mutation listed in Table A; or (ii) at least E421R, D425K, 545K E149K, N78H, A112K, T548R, and at least one other mutation listed in Table A2.

6. The IscB protein of any one of claim 1-5 comprising: an amino acid sequence that is at least 85%, at least 90%, at least 92%, at least 95%, at least 96%, at least 97%, at least 98%, at least 99%, or at least 99.5% identical to the amino acid sequence of an IscB as provided by SEQ ID NO: 2 or 8.

7. The IscB protein of any one of claims 1-6, wherein the IscB exhibits an increased activity on a target sequence that does not comprise the canonical TAM (5′-ATAAA-3′) at its 3′ end as compared to IscB as provided by SEQ ID NO: 2 or 8.

8. The IscB protein of any one of claims 1-7, wherein the IscB protein exhibits an activity on a target sequence having a 3′ end that is not directly adjacent to the canonical TAM sequence (5′-ATAAA-3′) that is at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of IscB as provided by SEQ ID NO: 2 or 8 on the same target sequence.

9. The IscB protein of any one of claims 6-8, wherein the activity is measured by a nuclease assay, a deamination assay, or a transcriptional activation assay.

10. The IscB protein of any one of claims 1-3, wherein the IscB protein comprises: (i) a E409R, D413K, and 533K mutations of the amino acid sequence provided in SEQ ID NO: 2; or (ii) a E421R, D425K, and 545K mutations of the amino acid sequence provided in SEQ ID NO: 8.

11. The IscB protein of any one of claim 1-3 or 10, wherein the IscB protein comprises: (i) a E137K, N66H, A100K, and/or T536R mutation of the amino acid sequence provided in SEQ ID NO: 2; or (ii) a E149K, N78H, A112K, and/or T548R mutation of the amino acid sequence provided in SEQ ID NO: 8.

12. The IscB protein of claim 11, wherein the IscB protein comprises: (i) E409R, D413K, 533K E137K, N66H, A100K, and T536R mutations of the amino acid sequence provided in SEQ ID NO: 2; or (ii) E421R, D425K, 545K E149K, N78H, A112K, and T548R mutations of the amino acid sequence provided in SEQ ID NO: 8.

13. The IscB protein of any one of claims 1-11, wherein the IscB exhibits an increased activity on a target sequence comprising a TAM sequence selected from the group consisting of RTAAA, ATRAA, ATRMM, ATRA, ATRRA, ATARA, ATRRAC, ATAAAC, ATARM, ATRAM, ATRRM, ATRMA, ATANA, ATRR, ATR, ATGAC, ATAA, ATAR, ATGMC, ATGAA, and ATRAR at its 3′ end as compared to IscB as provided by SEQ ID NO: 2 or 8, wherein R is a purine and M is A or C.

14. The IscB protein of claim 13, wherein the IscB comprises: (i) at least one, at least two, at least 3 or at least 4 mutations selected from the group consisting of variant H478X, H479N, I564X, T565X, H479C, and H479M of the amino acid sequence provided in SEQ ID NO: 2, wherein X is any amino acid; or (ii) at least one, at least two, at least 3 or at least 4 mutations selected from the group consisting of variant H490X, H491N, I576X, T577X, H491C, and H491M of the amino acid sequence provided in SEQ ID NO: 8, wherein X is any amino acid

15. The IscB protein of claim 14, wherein the IscB comprises at least one, at least two, at least 3 or at least 4 mutations selected from the group consisting of variant H478N, H479N, I564K, I564H, I564N, I564Q, I564C, I564M, I564T, I564D, T565R, T565K, T565H, T565C, T565D, T565G, H478C, H478P, H479C, and H479M of the amino acid sequence provided in SEQ ID NO: 2; or (ii) H490N, H491N, I576K, I576H, I576N, I576Q, I576C, I576M, I576T, I576D, T577R, T577K, T577H, T577C, T577D, T577G, H490C, H490P, H491C, and H491M of the amino acid sequence provided in SEQ ID NO: 8.

16. The IscB protein of any one of claims 1-15, wherein the IscB exhibits an increased activity on a target sequence comprising a TAM sequence is ATR.

17. The IscB protein of claim 13, comprising at least 3, at least 4, at least 5, at least 6, at least 7, at least 8, at least 9, at least 10 or at least 11 amino sequences swap at position 560-571 of SEQ ID NO: 2, or at least 3, at least 4, at least 5, at least 6, at least 7, at least 8, at least 9, at least 10 or at least 11 amino sequences swap at position 572-583 of SEQ ID NO: 8.

18. The IscB protein of claim 17, comprising at least 3, at least 4, at least 5, or at least 6 amino sequences swap at position Q563-K568 of SEQ ID NO: 2 or at least 3, at least 4, at least 5, or at least 6 amino sequences swap at position Q575-K580 of SEQ ID NO: 8.

19. The IscB protein of claim 18, comprising six amino sequences swap at position Q563-K568 of SEQ ID NO: 2 or at position is Q575-K580 of SEQ ID NO: 8 is selected from QTSGGR, RSSGGK, QQSKGR, QQGRGR, QQNKGR, and QKDKGA.

20. The IscB protein of claim 19, wherein the IscB exhibits an increased activity on a target sequence comprising a TAM sequence is ATR.

21. The IscB protein of claim 17, comprising at least 7, at least 8, at least 9, at least 10 or at least 11 amino sequences swap at position 560-571 of SEQ ID NO: 2, or at position 572-583 of SEQ ID NO: 8.

22. The IscB protein of claim 18, comprising the amino acid sequence MSGQHCHGAYLR, LSGNSHYGQYYM, MTGQHCHGNYFR, MTGQQNKGDRYF, KSGQLTNGQYFR, LKGNACNGAYFH, SSGTHAKSYFR, LGGQKNNGVYFY, VTGSSGAGKYVR, VRSVHSYGKYVR, VQGIKNRGRYLH, LRGTSSYGKTVL, VKGVFNYGKWVR, VKGKHNDRWI, VKGVQNRGKYIK, IKGSHCNGSRLI, SGGVMSYGRYIT, SKGNKNKGKSIL, AIGTHCKGKRVI, VKGVFNRGTWVR, CKGMHSYGRYVL, IGGTQNKGRYAI, or TKGTHCKGTRIM at position M560-R571 of SEQ ID NO: 2.

23. The IscB protein of claim 20, wherein the IscB exhibits an increased activity on a target sequence comprising a TAM sequence is ATRR.

24. The IscB protein of any one of claims 1-13, wherein the IscB exhibits an increased activity on a target sequence comprising a TAM sequence is RTAAA.

25. The IscB protein of claim 24, wherein the IscB comprises a mutation at position 478 and/or 479 of the amino acid sequence provided in SEQ ID NO: 2, or at position 490 and/or 491 of the amino acid sequence provided in SEQ ID NO: 2

26. The IscB protein of claim 25, wherein the IscB comprises: (i) a mutation H478N, H479N, H478C and/or H478P of the amino acid sequence provided in SEQ ID NO: 2; or (ii) a mutation H490N, H491N, H490C and/or H490P of the amino acid sequence provided in SEQ ID NO: 8.

27. The IscB protein of any one of claims 1-26, wherein the IscB protein exhibits lower off-target activity as compared to an off-target activity of the IscB domain as provided by SEQ ID NO: 2 or 8.

28. The IscB protein of any one of claims 1-27, wherein the IscB protein further comprises a nuclear localization signal (NLS) sequence.

29. The IscB protein of claim 28, wherein the NLS comprises an amino acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity with any one of the NLS sequences show in in Table C, or a homolog or ortholog thereof.

30. The IscB protein of claim 28 or 29, wherein the NLS is linked to N-terminal of the IscB protein.

31. The IscB protein of claim 28 or 29, wherein the NLS is linked to C-terminal of the IscB protein.

32. A fusion protein comprising (i) the IscB protein of any one of claims 1-31 and (ii) an effector domain.

33. The fusion protein of claim 32, wherein the effector domain is a domain that comprises nuclease activity, nickase activity, recombinase activity, deaminase activity, methyltransferase activity, methylase activity, acetylase activity, acetyltransferase activity, transcriptional activation activity, or transcriptional repression activity.

34. The fusion protein of claim 32 or 33, wherein the effector domain is a nucleic acid editing domain.

35. The fusion protein of claim 32 or 33, wherein effector domain comprises a deaminase domain, a reverse transcriptase, or a functional fragment thereof.

36. The fusion protein of claim 35 wherein the deaminase domain is a cytidine deaminase domain.

37. The fusion protein of claim 36, wherein the cytidine deaminase domain is an apolipoprotein B mRNA-editing complex (APOBEC) family deaminase.

38. The fusion protein of claim 36 or 37, wherein the cytidine deaminase domain is at least 80%, at least 85%, at least 90%, at least 92%, at least 95%, at least 96%, at least 97%, at least 98%, at least 99%, at least 99.5%, or 100% identical to the cytidine deaminase domain of any one of the sequences in Table C.

39. The fusion protein of claim 35, wherein the deaminase domain is an adenosine deaminase domain.

40. The fusion protein of claim 35, further comprising a second adenosine deaminase.

41. The fusion protein of claim 36, wherein the first adenosine deaminase domain and the second adenosine deaminase domain comprises an ecTadA domain, or variant thereof.

42. The fusion protein of claim 36 or 37, wherein the first adenosine deaminase domain and the second adenosine deaminase domain comprise independently an amino acid sequence that is at least 80%, at least 85%, at least 90%, at least 92%, at least 95%, at least 96%, at least 97%, at least 98%, at least 99%, at least 99.5%, or 100% identical to the cytidine deaminase domain of any one of the sequences in Table D1.

43. The fusion protein of any one of claims 32-42, wherein the fusion protein further comprises a uracil glycosylase inhibitor (UGI) domain.

44. The fusion protein of claim 43, wherein the UGI domain comprises an amino acid sequence having is at least 80%, at least 85%, at least 90%, at least 92%, at least 95%, at least 96%, at least 97%, at least 98%, at least 99%, at least 99.5%, or 100% identical to any one of the sequences in Table E.

45. The fusion protein of any one of claims 43-44, wherein the fusion protein further comprises a second UGI domain.

46. A ωRNA comprising a conserved portion of a ωRNA (parent ωRNA) comprising one or more stem-loop structures, wherein at least one of the stem-loop structures comprises a modification, and wherein a conserved portion of the ωRNA is shorter than the conserved portion of the parent ωRNA and is capable of binding with a transposon family encoded nuclease or a homolog or ortholog thereof.

47. The ωRNA of claim 46, wherein the conserved region of the ωRNA comprises at least two stem-loop structures and wherein said modification is in a stem-loop structure near to the 5′-end (5′-stem-loop) of the conserved region ωRNA.

48. The ωRNA of claim 47, wherein said modification comprises deletion of from about 3 to about 46 nucleotides from the 5′-stem-loop.

49. The ωRNA of claim 47 or 48, wherein said modification comprises deletion of 3, 7, 11, 15, 18, 22, 26, 30, 34, 37 or 41 nucleotides from the 5′-stem-loop.

50. The ωRNA of claim 4, wherein said modification comprises deletion of 15, 18, 30, 37 or 41 nucleotides from the 5′-stem-loop.

51. The ωRNA of claim 47 or 48, wherein said modification comprises deletion of 8, 12, 16, 20, 23, 27, 31, 35, 39, 42 or 46 nucleotides from the 5′-stem-loop.

52. The ωRNA of claim 51, wherein said modification comprises deletion of 20, 23, 35, 42 or 46 nucleotides from the 5′-stem-loop.

53. The ωRNA of any one of claims 47-52, wherein nucleotides at position 12-57, 14-55, 16-54, 18-52, 20-50, 22-48, 24-46, 26-45, 28-43, 30-41 or 32-39 are deleted (counting from 5′-end, e.g., 5′-end of the conserved region of the parent ωRNA).

54. The ωRNA of claim 53, wherein nucleotides at position 12-57, 14-55, 18-52, 24-46, or 26-45 are deleted (counting from 5′-end, e.g., 5′-end of the conserved region of the parent ωRNA).

55. The ωRNA of any one of claims 47-54, wherein the deleted nucleotides are replaced with a linker (e.g., a loop portion or pin-loop).

56. The ωRNA of claim 55, wherein the linker is single-stranded nucleic acid comprising from about from about 4 nucleotides to about 15 nucleotides.

57. The ωRNA of any one of claims 47-56, wherein a loop portion (pin-loop) of the 5′-stem-loop comprises from 4, 5 or 6 nucleotides.

58. The ωRNA of any one of claims 47-57, wherein a loop portion of the 5′-stem-loop comprises the nucleotide sequence GAAA or GAAAA.

59. The ωRNA of any one of claims 47-58, wherein at least a portion of the 5′-stem-loop is within 45 nucleotides, counting from 5′-end, e.g., 5′-end of the conserved region of the parent ωRNA.

60. The ωRNA of any one of claims 47-59, wherein at least a portion of the stem of the 5′-stem-loop is within 25 nucleotides, counting from 5′-end, e.g., 5′-end of the conserved region of the parent ωRNA.

61. The ωRNA of any one of claims 47-60, wherein a stem portion of the 5′-stem-loop comprises a double-stranded region comprising from about 8 nucleotide base-pairs to about 25 nucleotide base-pairs.

62. The ωRNA of any one of claims 47-61, wherein a stem portion of the 5′-stem-loop comprises a double-stranded region comprising about 9 nucleotide base-pairs, about 10 nucleotide base-pairs, about 11 nucleotide base-pairs, about 12 nucleotide base-pairs, about 13 nucleotide base-pairs, about 14 nucleotide base-pairs, about 15 base pairs nucleotide base-pairs, about 16 base pairs nucleotide base-pairs, about 17 base pairs nucleotide base-pairs, about 18 base pairs nucleotide base-pairs, about 19 base pairs nucleotide base-pairs, or about 20 base pairs nucleotide base-pairs.

63. The ωRNA of any one of claims 47-62, wherein a stem portion of the 5′-stem-loop comprises a double-stranded region comprising about 9 nucleotide base-pairs, about 10 nucleotide base-pairs, about 11 nucleotide base-pairs, or about 12 nucleotide base-pairs.

64. The ωRNA of any one of claims 47-63, wherein a stem portion of the 5′-stem-loop comprises a double-stranded region comprises 10 nucleotide base-pairs.

65. The ωRNA of any one of claims 47-64, wherein the 5′-stem-loop comprises from about 20 nucleotides to about 65 nucleotides.

66. The ωRNA of any one of claims 47-65, wherein the 5′-stem-loop comprises about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, or about 45 nucleotides.

67. The ωRNA of any one of claims 47-66, wherein the 5′-stem-loop comprises 23, 24, 25, 26 or 27 nucleotides.

68. The ωRNA of any one of claims 47-67, wherein the 5′-stem-loop comprises a nucleic acid modification, optionally selected from the group consisting of nucleobase modifications, sugar modifications, inter-sugar linkage modifications (modified internucleoside linkages), conjugates (e.g., ligands), and any combinations thereof.

69. The ωRNA of claim 68, wherein a stem (double-stranded) region of the 5′-stem-loop comprises a nucleic acid modification.

70. The ωRNA of claim 68 or 69, wherein a loop region (pin-loop) of the 5′-stem-loop comprises a nucleic acid modification.

71. The ωRNA of any one of claims 68-70, wherein said nucleic acid modification is a modified nucleotide selected from the group consisting of 2′-O-methyl(2′-OMe) nucleotides, 2′-fluoro (2′-F) nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl(2′-MOE) nucleotides, 2′-O-allyl nucleotides, 2′-C-allyl ribose nucleotides, 2′-O-N-methylacetamido (2′-O-NMA) nucleotides, a 2′-O-dimethylaminoethoxyethyl(2′-O-DMAEOE) nucleotides, 2′-O-aminopropyl(2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a non-natural or modified nucleobase; or a combination thereof.

72. The ωRNA of any one of claims 68-71, wherein a stem (double-stranded) region of the 5′-stem-loop comprises a 2′-O-methyl modified nucleotide.

73. The ωRNA of any one of claims 68-71, wherein a stem (double-stranded) region of the 5′-stem-loop comprises a 2′-fluoro modified nucleotide.

74. The ωRNA of any one of claims 68-71, wherein a nucleotide in a stem (double-stranded) region of the 5′-stem-loop comprises a non-natural or modified nucleobase.

75. The ωRNA of any one of claims 68-74, wherein a stem (double-stranded) region of the 5′-stem-loop comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

76. The ωRNA of claim 68-75, wherein a loop (single-stranded) region (pin-loop) of the 5′-stem-loop comprises a 2′-O-methyl modified nucleotide.

77. The ωRNA of any one of claims 68-75, wherein a loop (single-stranded) region (pin-loop) of the 5′-stem-loop comprises a 2′-fluoro modified nucleotide.

78. The ωRNA of any one of claims 68-77, wherein a nucleotide in a loop (single-stranded) (pin-loop) region of the 5′-stem-loop comprises a non-natural or modified nucleobase.

79. The ωRNA of any one of claims 68-78, wherein a loop (single-stranded) (pin-loop) region of the 5′-stem-loop comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH-C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

80. The ωRNA of any one of claims 47-79, wherein a stem (double-stranded) region of the 5′-stem-loop comprises a duplex stabilizing modification, optionally the duplex stabilizing modification is 2′-F nucleotide, 2′-OMe nucleotide, 2′-methoxyethyl nucleotide, 2,6-diaminopurine nucleotide, 5-methyl cytidine, N4-ethyl cytidine, 5-propynyl cytidine, 5-propynyl uridine, 5-hydroxybutynl-2′-deoxyuridine, 8-aza-7-deazaguanosine, a locked nucleic acid (LNA), and/or covalent cross-linking of two strands of the duplex.

81. The ωRNA of any one of claims 47-80, wherein two nucleotides in the stem of the 5′-loop-stem are covalently linked to each other via a position other than 5′ and 3′.

82. The ωRNA of any one of claims 46-81, wherein the conserved region of the ωRNA comprises at least two stem-loop structures and wherein said modification is in a stem-loop structure proximal to the 3′-end (3′-stem-loop) of the conserved region of the ωRNA.

83. The ωRNA of claim 82, wherein said modification comprises deletion of from about 1 to about 22 nucleotides from the 3′-stem-loop.

84. The ωRNA of claim 82 or 83, wherein said modification comprises deletion of 2, 6, 8, 10, 12, 16, or 20 nucleotides from the 3′-stem-loop.

85. The ωRNA of claim 84, wherein said modification comprises deletion of 2, 10, 12, or 16 nucleotides from the 3′-stem-loop.

86. The ωRNA of claim 82-85, wherein said modification comprises deletion of 2, 10, 12 or 16 nucleotides from the 5′-stem-loop.

87. The ωRNA of claim 86, wherein said modification comprises deletion of 2, 10, 12, or 16 nucleotides from the 3′-stem-loop.

88. The ωRNA of any one of claims 82-87, wherein nucleotides at position 159-172, 160-171, 161-170, 163-168, 158-173, 156-175 or 154-177 are deleted (counting from 5′-end, e.g., of the parent ωRNA).

89. The ωRNA of claim 88, wherein nucleotides at position 159-172, 163-168, 158-173, or 156-175 are deleted (counting from 5′-end, e.g., of the parent ωRNA).

90. The ωRNA of any one of claims 82-89, wherein the deleted nucleotides in the 3′-stem-loop are replaced with a linker (e.g., a loop portion or pin-loop).

91. The ωRNA of claim 90, wherein the linker is a single-stranded nucleic acid comprising from about from about 4 nucleotides to about 15 nucleotides.

92. The ωRNA of any one of claims 82-91, wherein a loop portion of the 5′-stem-loop comprises from 4, 5 or 6 nucleotides.

93. The ωRNA of any one of claims 82-92, wherein a loop portion of the 5′-stem-loop comprises the nucleotide sequence GAAA or GAAAA.

94. The ωRNA of any one of claims 82-93, wherein at least a portion of the 3′-stem-loop is within 50 nucleotides, counting from 3′-end of the modified ωRNA.

95. The ωRNA of any one of claims 82-94, wherein at least a portion of the stem of the 5′-stem-loop is within 45 nucleotides, counting from 3′-end of the modified ωRNA.

96. The ωRNA of any one of claims 82-95, wherein a stem portion of the 3′-stem-loop comprises a double-stranded region comprising from about 3 nucleotide base-pairs to about 10 nucleotide base-pairs.

97. The ωRNA of any one of claims 82-96, wherein a stem portion of the 3′-stem-loop comprises a double-stranded region comprising about 3 nucleotide base-pairs, about 4 nucleotide base-pairs, about 5 nucleotide base-pairs, about 6 nucleotide base-pairs, about 7 nucleotide base-pairs, or about 8 nucleotide base-pairs.

98. The ωRNA of any one of claims 82-97, wherein a stem portion of the 3′-stem-loop comprises a double-stranded region comprising about 3 nucleotide base-pairs, about 4 nucleotide base-pairs, about 5 nucleotide base-pairs, or about 6 nucleotide base-pairs.

99. The ωRNA of any one of claims 82-98, wherein a stem portion of the 5′-stem-loop comprises a double-stranded region comprises 3 or 4 nucleotide base-pairs.

100. The ωRNA of any one of claims 82-99, wherein the 3′-stem-loop comprises from about 8 nucleotides to about 25 nucleotides.

101. The ωRNA of any one of claims 78-100, wherein the 3′-stem-loop comprises about 9, about 10, about 11, about 12, about 13, about 14, about 15, about 16, about 17, about 18, about 19, or about 20 nucleotides.

102. The ωRNA of any one of claims 82-101, wherein the 3′-stem-loop comprises 9, 10, 11, 12, 13, 14 or 15 nucleotides.

103. The ωRNA of any one of claims 78-102, wherein the 3′-stem-loop comprises a nucleic acid modification, optionally selected from the group consisting of nucleobase modifications, sugar modifications, inter-sugar linkage modifications (modified internucleoside linkages), conjugates (e.g., ligands), and any combinations thereof.

104. The ωRNA of claim 103, wherein a stem (double-stranded) region of the 3′-stem-loop comprises a nucleic acid modification.

105. The ωRNA of claim 103 or 104, wherein a loop (single-stranded) region of the 3′-stem-loop comprises a nucleic acid modification.

106. The ωRNA of any one of claims 103-105, wherein said nucleic acid modification is a modified nucleotide selected from the group consisting of 2′-O-methyl(2′-OMe) nucleotides, 2′-fluoro (2′-F) nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl(2′-MOE) nucleotides, 2′-O-allyl nucleotides, 2′-C-allyl ribose nucleotides, 2′-O-N-methylacetamido (2′-O-NMA) nucleotides, a 2′-O-dimethylaminoethoxyethyl(2′-O-DMAEOE) nucleotides, 2′-O-aminopropyl(2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; a non-natural or modified nucleobase; or a combination thereof.

107. The ωRNA of any one of claims 82-106, wherein a stem (double-stranded) region of the 3′-stem-loop comprises a 2′-O-methyl modified nucleotide.

108. The ωRNA of any one of claims 82-107, wherein a stem (double-stranded) region of the 3′-stem-loop comprises a 2′-fluoro modified nucleotide.

109. The RNA of any one of claims 82-108, wherein a nucleotide in a stem (double-stranded) region of the 5′-stem-loop comprises a non-natural or modified nucleobase.

110. The ωRNA of any one of claims 82-109, wherein a stem (double-stranded) region of the 3′-stem-loop comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal 3′-O—CH₂—O-5′), oxime, methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

111. The ωRNA of any one of claims 82-110, wherein a loop (single-stranded) region of the 5′-stem-loop comprises a 2′-O-methyl modified nucleotide.

112. The ωRNA of any one of claims 82-111, wherein a loop (single-stranded) region of the 5′-stem-loop comprises a 2′-fluoro modified nucleotide.

113. The ωRNA of any one of claims 82-112, wherein a nucleotide in a loop (single-stranded) region of the 3′-stem-loop comprises a non-natural or modified nucleobase.

114. The ωRNA of any one of claims 82-113, wherein a loop (single-stranded) region of the 5′-stem-loop comprises at least one (e.g., 1, 2, 3, 4, 5, or more) modified internucleoside linkage (e.g., a modified internucleoside linkage selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, and 5′->5′ internucleoside linkages, optionally the modified internucleoside linkage is phosphorothioate, imidp or MMI, more preferably the modified internucleoside linkage is phosphorothioate (PS).

115. The ωRNA of any one of claims 82-114, wherein two nucleotides in the stem of the 5′-loop-stem are covalently linked to each other via a position other than 5′ and 3′.

116. The ωRNA of any one of claims 82-115, wherein two nucleotides in the stem of the 5′-loop-stem are covalently linked to each other via a position other than 5′ and 3′.

117. The ωRNA of any one of claims 46-116, wherein the conserved region of the ωRNA comprises at least three stem-loop structures.

118. The ωRNA of any one of claims 46-117, wherein at least one (e.g., 1, 2, 3, 4, 5, 6, 7 or 8) nucleotide in a loop region of a stem-loop forms a base-pair with a nucleotide outside said stem-loop.

119. The ωRNA of claim 118, wherein at least one (e.g., one or both) member of the base-pair comprises a modification capable of enhancing base-pairing.

120. The ωRNA of claim 119, wherein the base-pair nucleotide in the loop comprises the modification capable of enhancing base-pairing.

121. The ωRNA of claim 119 or 120, wherein the base-pair nucleotide outside said stem-loop comprises the modification capable of enhancing base-pairing.

122. The ωRNA of any one of claims 119-121, wherein said modification capable of enhancing base-pairing is 2′-F modified nucleotide, 2′-OMe modified nucleotide, 2′-methoxyethyl modified nucleotide, 2,6-diaminopurine nucleotide, 5-methyl cytidine, N4-ethyl cytidine, 5-propynyl cytidine, 5-propynyl uridine, 5-hydroxybutynl-2′-deoxyuridine, 8-aza-7-deazaguanosine, or a locked nucleic acid (LNA).

123. The ωRNA of any one of claims 119-122, wherein said modification capable of enhancing base-pairing is in at an internal (i.e., not a 5′-stem-loop or 3′-stem-loop) stem-loop (internal-hairpin) of the ωRNA.

124. The ωRNA of any one of claims 119-123, wherein said modification capable of enhancing base-pairing is in a loop (single-stranded) region (pin-loop) of an internal (i.e., not a 5′-stem-loop or 3′-stem-loop) stem-loop (internal-hairpin) of the ωRNA.

125. The ωRNA of any one of claims 46-124, wherein the ωRNA (e.g., a conserved region) is from about 50 to about 90 nucleotides shorter than the parent ωRNA.

126. The ωRNA of any one of claims 46-125, wherein the RNA (e.g., a conserved region) is from about 55 to about 85 nucleotides shorter than the parent ωRNA.

127. The ωRNA of any one of claims 46-126, wherein the ωRNA (e.g., a conserved region) is about 60, about 61, about 62, about 63, about 64, about 65, about 66, about 67, about 68, about 69, about 70, about 71, about 72, about 73, about 74, about 75, about 76, about 77, about 78, about 79, or about 80 nucleotides shorter than the parent ωRNA.

128. The ωRNA of any one of claims 46-127, wherein the ωRNA (e.g., a conserved region) is from about 130 nucleotides to about 190 nucleotides in length.

129. The ωRNA of any one of claims 46-128, wherein the ωRNA (e.g., a conserved region) is from about 135 nucleotides to about 180 nucleotides in length.

130. The ωRNA of any one of claims 46-129, wherein the ωRNA (e.g., a conserved region) is from about 140 nucleotides to about 170 nucleotides in length.

131. The ωRNA of any one of claims 46-130, wherein the ωRNA (e.g., a conserved region) is from about 140, about 141, about 142, about 143, about 144, about 145, about 146, about 147, about 148, about 149, about 150, about 151, about 152, about 153, about 1454, about 155, about 156, about 157, about 158, about 159, or about 160 nucleotides in length.

132. The ωRNA of any one of claims 46-131, wherein the ωRNA comprises a 5′ end modification.

133. The ωRNA of any one of claims 46-132, wherein the ωRNA comprises a 3′ end modification.

134. The ωRNA of any one of claims 46-133, wherein the ωRNA comprises a 5′ end modification and a 3′ end modification.

135. The ωRNA of any one of claims 132-134, wherein the 5′ and/or 3′ end modification comprises a protective end modification.

136. The ωRNA of claim 135, wherein the protective end modification is a modified nucleotide selected from 2′-O-methyl(2′-OMe) modified nucleotide, 2′-O-(2-methoxyethyl)(2′-O-moe) modified nucleotide, a 2′-fluoro (2′-F) modified nucleotide, a phosphorothioate (PS) linkage between nucleotides, a 3′->3′ linkage between nucleotides, an inverted abasic modified nucleotide, or combinations thereof.

137. The ωRNA of any one of claims 132-136, wherein the 3′ end modification comprises any one of:
i. a modification of any one or more of the last 7, 6, 5, 4, 3, 2, or 1 nucleotides;
ii. one modified nucleotide;
iii. two modified nucleotides;
iv. three modified nucleotides;
v. four modified nucleotides;
vi. five modified nucleotides;
vii. six modified nucleotides; or
viii. seven modified nucleotides.

138. The ωRNA of any one of claims 132-137, wherein the 3′ end modification
i. a modified internucleoside linkage (e.g., PS, imidp or MMI linkage) between nucleotides;
ii. a 2′-OMe nucleotide;
iii. a 2′-O-MOE nucleotide;
iv. a 2′-F nucleotide;
v. a LNA nucleotide
vi. a 3′->3′ linkage between nucleotides;
vii. an inverted abasic nucleotide; and
viii. a combination of one or more of (i.)-(vii.).

139. The ωRNA of any one of claims 132-138, wherein the 3′ end modification comprises one or more of:
i. a modified internucleoside linkage (e.g., PS, imidp or MMI linkage) between the last and second to last nucleotides;
ii. a modified internucleoside linkage (e.g., PS, imidp or MMI linkage) between the last and second to last nucleotide and a PS linkage between the second and third to last nucleotides;
iii. modified internucleoside linkages (e.g., PS and/or MMI linkage) between any one or more of the last four nucleotides;
iv. modified internucleoside linkages (e.g., PS and/or MMI linkage) between any one or more of the last five nucleotides; or
v. modified internucleoside linkages (e.g., PS and/or MMI linkage) between any one or more of the last 2, 3, 4, 5, 6, 7, 8, 9, or 10 nucleotides.

140. The ωRNA of any one of claims 132-139, wherein the 3′ end modification comprises at least one 2′-F nucleotide, 2′-OMe nucleotide, 2′-O-MOE nucleotide, inverted abasic nucleotide or a 3′->3′ internucleoside linkage.

141. The ωRNA of any one of claims 132-140, wherein the 3′ end modification comprises:
i. a modification of one or more of the last 1-7 nucleotides, wherein the modification is a modified internucleoside linkages (e.g., PS and/or MMI linkage), inverted abasic nucleotide, a 3′->3′ internucleoside linkage, 2′-OMe, 2′-O-MOE, 2′-F, LNA, or a combination thereof;
ii. a modification to the last nucleotide with 2′-OMe, 2′-O-MOE, 2′-F, LNA, or combinations thereof, and an optional one or two modified internucleoside linkages (e.g., PS and/or MMI linkage) to the next nucleotide;
iii. a modification to the last and/or second to last nucleotide with 2′-OMe, 2′-O-MOE, 2′-F, LNA, or combinations thereof, and optionally one or more modified internucleoside linkages (e.g., PS and/or MMI linkage); iv. a modification to the last, second to last, and/or third to last nucleotides with 2′-OMe, 2′-O-moe, 2′-F, or combinations thereof, and optionally one or more modified internucleoside linkages (e.g., PS and/or MMI linkage);
v. a modification to the last, second to last, third to last, and/or fourth to last nucleotides with 2′-OMe, 2′-O-MOE, 2′-F, LNA or combinations thereof, and optionally one or more modified internucleoside linkages (e.g., PS and/or MMI linkage); or
vi. a modification to the last, second to last, third to last, fourth to last, and/or fifth to last nucleotides with 2′-OMe, 2′-O-MOE, 2′-F, LNA, or combinations thereof, and optionally one or more modified internucleoside linkages (e.g., PS and/or MMI linkage).

142. The ωRNA of any one of claims 132-141, wherein the 3′ end modification comprises any one or more of:
(i) a 2′-OMe modified nucleotide at the last position, three consecutive 2′-O-MOE modified nucleotides immediately 5′ to the 2′-OMe modified nucleotide, and three consecutive PS linkages between the last three nucleotides;
(ii) five consecutive 2′-OMe modified nucleotides from the 3′ end of the 3′ terminus, and three PS linkages between the last three nucleotides;
(iii) an inverted abasic modified nucleotide at the last position;
(iv) an inverted abasic modified nucleotide at the last position, and three consecutive 2′-OMe modified nucleotides at the last three positions
(v) 15 consecutive 2′-OMe modified nucleotides from the 3′ end, five consecutive 2′-F modified nucleotides immediately 5′ to the 2′-OMe modified nucleotides, and three PS linkages between the last three nucleotides;
(vi) alternating 2′-OMe modified nucleotides and 2′-F modified nucleotides at the last 20 nucleotides, and three PS linkages between the last three nucleotides;
(vii) two or three consecutive 2′-OMe modified nucleotides, and three PS linkages between the last three nucleotides;
(viii) one PS linkage between the last and next to last nucleotides; and
(ix) 15 or 20 consecutive 2′-OMe modified nucleotides, and three PS linkages between the last three nucleotides.

143. The ωRNA of any one of claims 132-142, wherein the 5′ end modification comprises any one or more of:
i. one modified nucleotide;
ii. two modified nucleotides;
iii. three modified nucleotides;
iv. four modified nucleotides;
v. five modified nucleotides;
vi. six modified nucleotides; and
vii. seven modified nucleotides.

144. The ωRNA of any one claims 132-143, wherein the 5′ end modification comprises any one or more of: a modification of between 1 and 7, between 1 and 5, between 1 and 4, between 1 and 3, or between 1 and 2 nucleotides.

145. The ωRNA of any one of claims 132-144, wherein the 5′ end modification comprises any one or more of:
i. a modified internucleoside linkage (e.g., a phosphorothioate and/or MMI linkage) between nucleotides;
ii. a 2′-OMe nucleotide;
iii. a 2′-O-MOE nucleotide;
iv. a 2′-F nucleotide;
v. a LNA;
vi. a 3′->3′ linkage;
vii. an inverted abasic modified nucleotide;
viii. a deoxyribonucleotide;
ix. an inosine; and
x. combinations of one or more of (i.)-(ix.).

146. The ωRNA of any one of claims 132-145, wherein the 5′ end modification comprises:
i. 1, 2, 3, 4, 5, 6, and/or 7 modified internucleoside (e.g., a phosphorothioate and/or MMI) linkages between nucleotides; or
ii. about 1-2, 1-3, 1-4, 1-5, 1-6, or 1-7 modified internucleoside (e.g., a phosphorothioate and/or MMI) linkages between nucleotides.

147. The ωRNA of any one claims 132-146, wherein the 5′ end modification comprises:
i. there is one modified internucleoside (e.g., a phosphorothioate and/or MMI) linkage, and the linkage is between nucleotides 1 and 2;
ii. there are two modified internucleoside (e.g., a phosphorothioate and/or MMI) linkages, and the linkages are between nucleotides 1 and 2, and 2 and 3;
iii. there are modified internucleoside (e.g., a phosphorothioate and/or MMI) linkages between any one or more of nucleotides 1 and 2, 2 and 3, and 3 and 4;
iv. there are modified internucleoside (e.g., a phosphorothioate and/or MMI) linkages between any one or more of nucleotides 1 and 2, 2 and 3, 3 and 4, and 4 and 5;
v. there are modified internucleoside (e.g., a phosphorothioate and/or MMI) linkages between any one or more of nucleotides 1 and 2, 2 and 3, 3 and 4, 4 and 5, and 5 and 6;
vi. there are modified internucleoside (e.g., a phosphorothioate and/or MMI) linkages between any one or more of nucleotides 1 and 2, 2 and 3, 3 and 4, 4 and 5, 5 and 6, and 6 and 7; or
vii. there are modified internucleoside (e.g., a phosphorothioate and/or MMI) linkages between any one or more of nucleotides 1 and 2, 2 and 3, 3 and 4, 4 and 5, 5 and 6, 6 and 7, and 7 and 8.

148. The ωRNA of any one of claims 132-147, wherein the 5′ end modification comprises at least one 2′-OMe, 2′-O-MOE, inverted abasic, or 2′-F modified nucleotide.

149. The ωRNA of any one of claims 46-148, wherein the ωRNA further comprises a guide region capable of hybridizing with a target nucleic acid.

150. The ωRNA of any one of claims 46-149, wherein the ωRNA is hybridized with a second nucleic acid strand.

151. The ωRNA of claim 150, wherein the second strand comprises a guide region comprising a sequence capable of hybridizing with a target nucleic acid.

152. The ωRNA of any one of claims 149-151, wherein the guide region is from about 15 to about 30 nucleotides in length.

153. The ωRNA of any one of claims 149-152, wherein the guide region is from about 18 to about 25 nucleotides in length.

154. The ωRNA of any one of claims 149-153, wherein the guide region is from 18, 19, 20, 21, 22 or 23 nucleotides in length.

155. The ωRNA of any one of claims 149-154, wherein the guide region is at 5′-end of the ωRNA.

156. The ωRNA of any one of claims 149-154, wherein the guide region is at 3′-end of the ωRNA.

157. The ωRNA of any one of claims 149-156, wherein the guide region comprises a nucleic acid modification, optionally selected from the group consisting of nucleobase modifications, sugar modifications, inter-sugar linkage modifications (modified internucleoside linkages), conjugates (e.g., ligands), and any combinations thereof.

158. The ωRNA of claim 157, wherein said nucleic acid modification is a modified nucleotide selected from the group consisting of 2′-O-methyl(2′-OMe) nucleotides, 2′-fluoro (2′-F) nucleotides, bridged nucleic acid (BNA) nucleotides (e.g., 2′-O,4′-C-methylene (locked nucleic acid, LNA) nucleotides, 2′-O,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol acid, HNA) nucleotides, cyclohexene (Cyclohexene nucleic acid, CeNA) nucleotides, 2′-methoxyethyl(2′-MOE) nucleotides, 2′-O-allyl nucleotides, 2′-C-allyl ribose nucleotides, 2′-O-N-methylacetamido (2′-O-NMA) nucleotides, a 2′-O-dimethylaminoethoxyethyl(2′-O-DMAEOE) nucleotides, 2′-O-aminopropyl(2′-O-AP) nucleotides, 2′-F arabinose (2′-ara-F) nucleotides, threose (Threose nucleic acid, TNA) nucleotides, and acyclic nucleotides (e.g., unlocked nucleic acids (UNA) and 2,3-dihydroxylpropyl (glycol nucleic acid, GNA)); a modified internucleoside linkage; or a non-natural or modified nucleobase.

159. The ωRNA of any one of claims 149-156, wherein the guide region comprises:
i. a modification of one or more of nucleotides 1-7, wherein the modification is a modified internucleoside (e.g., phosphorothioate and/or MMI) linkage, 3′->3′ internucleoside linkage, inverted abasic nucleotide, 2′-OMe, 2′-O-MOE, 2′-F, 2′-H (a deoxyribonucleotide), an inosine, and/or combinations thereof;
ii. a modification to the first nucleotide of the guide region with 2′-OMe, 2′-O-MOE, 2′-F, 2′-H, an inosine, or combinations thereof, and an optional modified internucleoside (e.g., phosphorothioate and/or MMI) linkage to the next nucleotide;
iii. a modification to the first and/or second nucleotide of the guide region with 2′-OMe, 2′-O-MOE, 2′-F, 2′-H, an inosine, or combinations thereof, and optionally one or more modified internucleoside (e.g., phosphorothioate and/or MMI) linkages;
iv. a modification to the first, second, and/or third nucleotides of the guide region with 2′-OMe, 2′-O-MOE 2′-F, 2′-H, an inosine, or combinations thereof, and optionally one or more modified internucleoside (e.g., phosphorothioate and/or MMI) linkages;
v. a modification to the first, second, third, and/or fourth nucleotides of the guide region with 2′-OMe, 2′-O-MOE, 2′-F, 2′-H, an inosine, or combinations thereof, and optionally one or more modified internucleoside (e.g., phosphorothioate and/or MMI) linkages; or
vi. a modification to the first, second, third, fourth, and/or fifth nucleotides of the guide region with 2′-OMe, 2′-O-MOE, 2′-F, 2′-H, an inosine, or combinations thereof, and optionally one or more modified internucleoside (e.g., phosphorothioate and/or MMI) linkages.

160. The ωRNA of any one of claims 149-159, wherein the ωRNA comprises any one or more of:
i. 2′-OMe nucleotides at nucleotides 1, 2, and 3 of the guide region;
ii. 2′-OMe nucleotides at nucleotides 1, 2, and 3 of the guide region and modified internucleoside (e.g., phosphorothioate and/or MMI) linkages between nucleotides 1 and 2, 2 and 3, and 3 and 4 of the guide region;
iii. 2′-OMe nucleotides at nucleotides 1, 2, 3, 4, and 5 of the guide region;
iv. 2′-OMe nucleotides at nucleotides 1, 2, 3, 4, and 5 of the guide region and modified internucleoside (e.g., phosphorothioate and/or MMI) linkages between nucleotides 1 and 2, 2 and 3, 3 and 4, 4 and 5, and 5 and 6 of the guide region;
v. 2′-O-MOE modified nucleotides at nucleotides 1, 2, and 3 of the guide region;
vi. 2′-O-MOE modified nucleotides at nucleotides 1, 2, and 3 of the guide region and modified internucleoside (e.g., phosphorothioate and/or MMI) linkages between nucleotides 1 and 2, 2 and 3, and 3 and 4 of the guide region;
vii. an inverted abasic nucleotide at nucleotide 1 of the guide region;
viii. an inverted abasic nucleotide at nucleotide 1 of the guide region and 2′-OMe nucleotides at nucleotides 1, 2, and 3 of the guide region; and
ix. an inverted abasic nucleotide at nucleotide 1 of the guide region, 2′-OMe nucleotides at nucleotides 1, 2, and 3 of the guide region, and modified internucleoside (e.g., phosphorothioate and/or MMI) linkages between nucleotides 1 and 2, 2 and 3, 3 and 4, 4 and 5, and 5 and 6 of the variable region.

161. The ωRNA of any one of claims 46-160, wherein the ωRNA comprises one of more (e.g., 1, 2, 3, 4, 5, 6, 7, 8, 9, 10 or more including all) modified pyrimidine-A dinucleotide (modified YA site).

162. The ωRNA of any one of claims 46-161, wherein the ωRNA comprises at least one modified YA site in the guide region.

163. The ωRNA of any one claims 46-162, wherein the ωRNA comprises at least one modified YA site in a conserved region of the ωRNA.

164. The ωRNA of any one of claims 46-163, wherein the modified YA site comprises a 2′-fluoro nucleotide, optionally the pyrimidine of the YA site; a 2′-H nucleotide, optionally the pyrimidine of the YA site; a 2′-OMe nucleotide, optionally the pyrimidine of the YA site; a PS linkage between the nucleotides; or a combination thereof.

165. The ωRNA of any one of claims 46-164, wherein the ωRNA comprises:
i. a modified YA site at 1, 2, 3, 4, or 5 guide region YA sites;
ii a modified YA site at 1, 2, 3, 4, or 5 guide region YA sites, wherein the modification of at least one guide region YA site is different from any 5′ end modification of the ωRNA;
iii. a modified YA site at one or more guide region YA sites that are at or after nucleotide 8 from the 5′ end of the 5′ terminus;
iv. a modified YA site at one or more guide region YA sites that are within nucleotides 5-end, 6-end, 7-end, 8-end, 9-end, or 10-end from the 5′ end of the 5′ terminus;
v. a modified YA site at one or more guide region YA sites that are within 17, 16, 15, 14, 13, 12, 11, 10, or 9 nucleotides of the 3′ terminal nucleotide of the guide region;
vi. a modified YA site at a guide region YA site other than a 5′ end modification; or
vii. a modified YA site at a guide region YA site, wherein the modification of the guide region YA site comprises a modification at least one nucleotide located 5′ of the guide region YA site does not comprise.

166. The ωRNA of any one of claims 46-165, wherein the ωRNA comprises:
i. a modified YA site at two or more guide region YA sites that are at or after nucleotide 8 from the 5′-end;
ii. a modified YA site at two or more guide region YA sites that are within nucleotides 5-end, 6-end, 7-end, 8-end, 9-end, or 10-end from the 5′ end;
iii. a modified YA site at two or more guide region YA sites that are within 17, 16, 15, 14, 13, 12, 11, 10, or 9 nucleotides of the 3′ terminal nucleotide of the guide region;
iv. a modified YA site at two or more guide region YA sites other than a 5′ end modification; or
v. a modified YA site at a two or more guide region YA sites, wherein the modifications of the guide region YA sites comprise a modification at least one nucleotide located 5′ of the guide region YA site does not comprise.

167. The ωRNA of any one of claims 46-166, wherein the ωRNA comprises:
i. a modified YA site at three or more guide region YA sites that are at or after nucleotide 8 from the 5′ end of the 5′ terminus;
ii. a modified YA site at three or more guide region YA sites that are within nucleotides 5-end, 6-end, 7-end, 8-end, 9-end, or 10-end from the 5′ end of the 5′ terminus;
iii. a modified YA site at three or more guide region YA sites that are within 17, 16, 15, 14, 13, 12, 11, 10, or 9 nucleotides of the 3′ terminal nucleotide of the guide region;
a modified YA site at three or more guide region YA sites other than a 5′ end iv. modification; or
v. a modified YA site at a three or more guide region YA sites, wherein the modifications of the guide region YA sites comprise a modification at least one nucleotide located 5′ of the guide region YA site does not comprise.

168. The ωRNA of any one of claims 46-167, wherein the ωRNA retains at least 75% of the activity of the parent ωRNA.

169. The ωRNA of any one of claims 46-168, wherein the nuclease is a transposon family nuclease.

170. The ωRNA of claim 169, wherein nuclease is a IS200/IS605 transposon family encoded nuclease or a homolog or ortholog thereof.

171. The ωRNA of claim 170, wherein the nuclease is ISC (insertion sequences Cas9-like), TnpB, or a homolog or ortholog thereof.

172. The ωRNA of claim 171, wherein the nuclease is IscB nuclease, IsrB nuclease, TnpB nuclease, or a homolog or ortholog thereof, optionally, the nuclease is IscB nuclease or a homolog or ortholog thereof.

173. The ωRNA of any one of claims 46-172, wherein the nuclease is modified nuclease.

174. The ωRNA of any one of claims 46-173, wherein the ωRNA lacks from 1 to about 20 nucleotides present at 3′-end of the conserved portion of the parent ωRNA.

175. A vector comprising the ωRNA of any one of claims 46-174.

176. A composition comprising a ωRNA of any one of claims 46-174 or a polynucleotide encoding the same.

177. The composition of claim 176, wherein the composition is in form of a lipid nanoparticles (LNP) or a protein nanoparticles (PNP).

178. The composition of any one of claims 176-177, wherein the composition further comprises a nuclease or a polynucleotide encoding the nuclease.

179. The composition of claim 178, wherein the nuclease is a transposon family nuclease.

180. The composition of claim 178-179, wherein nuclease is a IS200/IS605 transposon family encoded nuclease or a homolog or ortholog thereof.

181. The composition of claim 180, wherein the nuclease is ISC, TnpB, or a homolog or ortholog thereof.

182. The composition of claim 181, wherein the nuclease is IscB, IsrB, TnpB nuclease, or a homolog or ortholog thereof, optionally, the nuclease is IscB or a homolog or ortholog thereof.

183. The composition of any one of claims 178-182, wherein the nuclease is a modified nuclease.

184. A pharmaceutical composition comprising an IscB protein of any one of claims 1-31, a fusion protein of any one of claims 32-45, a ωRNA of any one of claims 46-174, and/or a composition of any one of claims 176-183.

185. A DNA and RNA targeting system comprising: (i) a protein effector polypeptide or a polynucleotide encoding the protein effector polypeptide, optionally, the protein effector polypeptide has nuclease (e.g., endonuclease activity); and (ii) a ωRNA of any one of claims 46-174 or a polynucleotide encoding the ωRNA.

186. A DNA and RNA targeting composition comprising: (i) a protein effector polypeptide or a polynucleotide encoding the protein effector polypeptide, optionally, the protein effector polypeptide has nuclease (e.g., endonuclease activity); and (ii) a ωRNA of any one of claims 46-174 or a polynucleotide encoding the ωRNA.

187. The DNA and RNA targeting system of claim 185 or the DNA and RNA targeting composition of claim 186, wherein the nuclease is a transposon family nuclease.

188. The DNA and RNA targeting system or composition of claim 187, wherein nuclease is a IS200/IS605 transposon family encoded nuclease or a homolog or ortholog thereof.

189. The DNA and RNA targeting system or composition of claim 188, wherein the nuclease is ISC, TnpB, or a homolog or ortholog thereof.

190. The DNA and RNA targeting system or composition of claim 189, wherein the nuclease is IscB, IsrB, TnpB nuclease, or a homolog or ortholog thereof, optionally, the nuclease is IscB or a homolog or ortholog thereof.

191. The DNA and RNA targeting system or composition of any one of claims 185-190, wherein the nuclease is a modified nuclease.

192. The DNA and RNA targeting system or composition of any one of claims 185-191, wherein the protein effector polypeptide comprises a RuvC domain, and/or an HNH domain.

193. The DNA and RNA targeting system or composition of any one of claims 185-192, wherein the protein effector polypeptide comprises: (i) a split Ruv-C nuclease domain comprising RuvC-I, RuvC-II, and RuvC-III subdomains; and/or (ii) an HNH domain.

194. The DNA and RNA targeting system or composition of any one of claims 185-193, wherein the protein effector polypeptide comprises a PLMP domain and optionally a conserved C-terminal Y domain.

195. The DNA and RNA targeting system or composition of any one of claims 192-194, wherein the HNH domain is located in the RuvC domain (e.g., the HNH domain is located between RuvC-II and RuvC-III subdomains).

196. The DNA and RNA targeting system or composition of any one of claims 192-194, wherein the protein effector polypeptide comprises a HNH domain but no RuvC domain.

197. The DNA and RNA targeting system or composition of any one of claims 192-194, wherein the protein effector polypeptide comprises a RuvC (e.g., split RuvC) domain but no HNH domain.

198. The DNA and RNA targeting system or composition of any one of claims 192-197, wherein the protein effector polypeptide further comprises a REC domain.

199. The DNA and RNA targeting system or composition of claim 198, wherein the REC domain is downstream from the RuvC or HNH domain.

200. The DNA and RNA targeting system or composition of claim 198, wherein the REC domain is upstream from the RuvC or HNH domain.

201. The DNA and RNA targeting system or composition of any one of claims 185-200, wherein the protein effector polypeptide comprises from about 170 to about 1000 amino acids.

202. The DNA and RNA targeting system or composition of any one of claims 185-201, wherein the ωRNA comprises a guide sequence comprising a nucleotide sequence substantially complementary to at least a portion of a target nucleic acid (target sequence)

203. The DNA and RNA targeting system or composition of claim 202, wherein the target nucleic acid comprises a target adjacent motif (TAM) sequence 3′ of the target sequence.

204. The DNA and RNA targeting system of claim 203, wherein the TAM comprises the sequence NAC, where N is A, C, G, or T.

205. The DNA and RNA targeting system or composition of any one of claims 202-204, wherein the target nucleic acid is DNA.

206. The DNA and RNA targeting system or composition of any one of claims 185-205 further comprising a functional domain associated with the protein effector polypeptide.

207. The DNA and RNA targeting system or composition of claim 206, wherein the functional domain has transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, detectable activity, or any combination thereof.

208. The DNA and RNA targeting system or composition of any one of claims 185-207, further comprising a serine or tyrosine recombinase.

209. The DNA and RNA targeting system or composition of any one of claims 185-208, further comprising a homologous recombination donor template comprising a donor sequence for insertion into a target polynucleotide.

210. An engineered cell comprising a DNA and RNA targeting system or composition of any one of claims 185-209.

211. A complex comprising the fusion protein of any one of claims 32-45, and a guide RNA bound to the IscB protein.

212. A complex comprising the fusion protein of any one of claims 32-45, and a guide RNA comprising the ωRNA of any one of claims 46-174 bound to the IscB protein.

213. The complex of claim 211 or 212, wherein the guide RNA is about 15-100 nucleotides long and comprises a sequence of at least 10 contiguous nucleotides that is complementary to a target sequence.

214. The complex of any one of claims 211-213, wherein the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long.

215. The complex of any one of claims 211-214, wherein the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

216. The complex of any one of claims 211-215, wherein the target sequence is a DNA sequence.

217. The complex of claim 216, wherein the target sequence is a sequence in the genome of a mammal.

218. The complex of claim 217, wherein the target sequence is a sequence in the genome of a human.

219. The complex of any one of claims 211-218, wherein the target sequence comprises a sequence associated with a disease or disorder.

220. The complex of claim 219, wherein the target sequence comprises a point mutation associated with a disease or disorder.

221. The complex of claim 220, wherein the complex edits a point mutation in the target sequence.

222. The complex of claim 221, wherein the point mutation is located between about 10 to about 20 nucleotides upstream of the TAM in the target sequence.

223. The complex of claim 221 or 222, wherein the target sequence comprises a T to C point mutation.

224. The complex of claim 223, wherein the complex deaminates the target C point mutation, and wherein the deamination results in a sequence that is not associated with a disease or disorder.

225. The complex of claim 224, wherein the target C point mutation is present in the DNA strand that is not complementary to the guide RNA.

226. The complex of claim 221 or 222, wherein the target sequence comprises a G to A point mutation.

227. The complex of claim 226, wherein the complex deaminates the target A point mutation, and wherein the deamination results in a sequence that is not associated with a disease or disorder.

228. The complex of claim 227, wherein the target A point mutation is present in the DNA strand that is not complementary to the guide RNA.

229. The complex of any one of claims 211-228, wherein the complex exhibits increased deamination efficiency of a point mutation in a target sequence that does not comprise the canonical TAM (5′-ATAAA-3′) at its 3′ end as compared to the deamination efficiency of a complex comprising IscB protein as provided by SEQ ID NO: 2.

230. The complex of claim 229, wherein the complex exhibits increased deamination efficiency of a point mutation in a target sequence having a 3′ end that is not directly adjacent to the canonical TAM sequence (5′-ATAAA-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the deamination efficiency of complex comprising the IscB protein as provided by SEQ ID NO: 2 on the same target sequence.

231. The complex of any one of claims 224-230, wherein a deamination activity is measured using a deamination assay, PCR, or sequencing.

232. The complex of any one of claims 211-231, wherein the complex produces fewer indels in a target sequence that does not comprise the canonical TAM (5′-ATAAA-3′) at its 3′ end as compared to the amount of indels produced by a complex comprising IscB protein as provided by SEQ ID NO: 2.

233. The complex of claim 232, wherein the complex produces fewer indels in a target sequence having a 3′ end that is not directly adjacent to the canonical TAM sequence (5′-ATAAA-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold lower as compared to the amount of indels produced by a complex comprising IscB protein as provided by SEQ ID NO: 2 on the same target sequence.

234. The complex of any one of claims 232-233, wherein indels are measured using high-throughput sequencing.

235. The complex of any one of claims 211-234, wherein the complex exhibits a decreased off-target activity as compared to the off-target activity of a complex comprising IscB protein as provided by SEQ ID NO: 2.

236. The complex of claim 235, wherein the off-target activity of the complex is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold decreased as compared to the off-target activity of a complex comprising IscB protein as provided by SEQ ID NO: 2.

237. The complex of any one of claims 211-236, wherein the target sequence is in the genome of an organism.

238. The complex of claim 237, wherein the organism is a prokaryote.

239. The complex of claim 237, wherein the prokaryote is a bacterium.

240. The complex of claim 237, wherein the organism is a eukaryote.

241. The complex of claim 237, wherein the organism is a plant or fungus.

242. The complex of claim 237, wherein the organism is a vertebrate.

243. The complex of claim 242, wherein the vertebrate is a mammal.

244. The complex of claim 243, wherein the mammal is a human.

245. The complex of claim 237, wherein the organism is a cell.

246. The complex of claim 245, wherein the cell is a human cell.

247. A method comprising contacting a nucleic acid with the fusion protein of any one of claims 32-45, and with a guide RNA, wherein the guide RNA is about 15-100 nucleotides long and comprises a sequence of at least 10 contiguous nucleotides that is complementary to a target sequence.

248. A method comprising contacting a cell with the fusion protein of any one of claims 32-45, and with a guide RNA, wherein the guide RNA is about 15-100 nucleotides long and comprises a sequence of at least 10 contiguous nucleotides that is complementary to a target sequence.

249. A method comprising contacting a nucleic acid with the fusion protein of any one of claims 32-45, and with a guide RNA comprising the ωRNA of any one of claims 46-174.

250. A method comprising contacting a cell with the fusion protein of any one of claims 32-45, and with a guide RNA comprising the ωRNA of any one of claims 46-174.

251. A method comprising contacting a nucleic acid with the complex of any one of claims 211-246.

252. A method comprising contacting a cell with the complex of any one of claims 211-246.

253. The method of any one of claims 247-252, wherein the contacting is performed in vitro.

254. The method of any one of claims 247-253, wherein the contacting is performed in vivo.

255. A method comprising administering to a subject the fusion protein of any one of claims 32-45, and a guide RNA, wherein the guide RNA is about 15-100 nucleotides long and comprises a sequence of at least 10 contiguous nucleotides that is complementary to a target sequence.

256. A method comprising administering to a subject the fusion protein of any one of claims 32-45, and a guide RNA comprising the ωRNA of any one of claims 46-174.

257. A method comprising administering to a subject the complex of any one of claims 211-246 or the DNA and RNA targeting system or composition of any one of claims 185-209.

258. The method of any one of claims 247-257, wherein the target sequence of the nucleic acid is a DNA sequence.

259. The method of any one of claims 247-258, wherein the 3′ end of the target sequence is not immediately adjacent to the canonical TAM sequence (5′-ATAAA-3′).

260. The method of any one of claims 247-259 wherein the target sequence comprises a sequence associated with a disease or disorder.

261. The method of claim 260, wherein the target DNA sequence comprises a point mutation associated with a disease or disorder.

262. The method of claim 261, wherein the activity of the fusion protein, or the activity of the complex, results in a correction of the point mutation.

263. The method of any one of claims 259-262, wherein the target DNA sequence comprises a T to C point mutation associated with a disease or disorder, and wherein the deamination of the mutant C base results in a sequence that is not associated with a disease or disorder.

264. The method of claim 263, wherein the target DNA sequence encodes a protein and wherein the point mutation is in a codon and results in a change in the amino acid encoded by the mutant codon as compared to the wild-type codon.

265. The method of claim 263 or 264, wherein the deamination of the mutant C results in a change of the amino acid encoded by the mutant codon.

266. The method of claim 263, wherein the deamination of the mutant C results in the codon encoding the wild-type amino acid.

267. The method of any one of claims 261-262, wherein the target DNA sequence comprises a G to A point mutation associated with a disease or disorder, and wherein the deamination of the mutant A base results in a sequence that is not associated with a disease or disorder.

268. The method of claim 267, wherein the target DNA sequence encodes a protein and wherein the point mutation is in a codon and results in a change in the amino acid encoded by the mutant codon as compared to the wild-type codon.

269. The method of claim 268, wherein the deamination of the mutant A results in a change of the amino acid encoded by the mutant codon.

270. The method of claim 267, wherein the deamination of the mutant A results in the codon encoding the wild-type amino acid.

271. The method of any one of claims 247-270, wherein the contacting is in vivo in a subject.

272. The method of claim 271, wherein the subject has or has been diagnosed with a disease or disorder.

273. The method of claim 271 or 272, wherein the disease or disorder is a proliferative disease, a genetic disease, a neoplastic disease, a metabolic disease, or a lysosomal storage disease.

274. A kit comprising a nucleic acid construct, comprising:
(a) a nucleic acid sequence encoding the fusion protein of any one of claims 32-45; and
(b) a heterologous promoter that drives expression of the sequence of (a).

275. A kit comprising a nucleic acid construct, comprising:
(a) a nucleic acid sequence encoding the complex of any one of claims 211-246; and (b) a heterologous promoter that drives expression of the sequence of (a).

276. The kit of 274 or 275, further comprising an expression construct encoding a guide RNA backbone, wherein the construct comprises a cloning site positioned to allow the cloning of a nucleic acid sequence identical or complementary to a target sequence into the guide RNA backbone.

277. The kit of any one of claims 274-276, further comprising an expression construct encoding a guide RNA comprising the ωRNA of any one of claims 46-174.

278. A polynucleotide encoding the fusion protein of any one of claims 32-45 or the complex of any one of claims 211-246 or the DNA and RNA targeting system or composition of any one of claims 185-209.

279. A vector comprising a polynucleotide of claim 278.

280. The vector of claim 279, wherein the vector comprises a heterologous promoter driving expression of the polynucleotide encoding the fusion protein or the polynucleotide encoding the complex.

281. A method comprising contacting a cell with the vector of claim 279 or 280.

282. The method of claim 281, wherein the vector is transfected into the cell.

283. The method of claim 282, wherein the vector is transfected into the cell using electroporation, heat shock, or a composition comprising a cationic lipid.

284. A cell comprising the fusion protein of any one of claims 32-45, or a nucleic acid molecule encoding the fusion protein of any one of claims 32-45.

285. A cell comprising the complex of any one of claims 211-246, or a nucleic acid molecule encoding the complex of any one of claims 211-246.

286. A cell comprising the vector of claim 279 or 280.

287. A DNA and RNA targeting composition comprising an IscB protein of any one of claims 1-31 and a guide nucleic acid capable of binding with the IscB protein, wherein the guide nucleic acid comprises a guide sequence complementary to target sequence and capable of directing the IscB protein to a target polynucleotide.

288. A DNA and RNA targeting composition comprising an IscB protein of any one of claims 1-31 and a guide nucleic acid comprising the ωRNA of any one of claims 46-174.

289. The composition of claim 287 or 288, wherein the guide is from about 10 nucleotides to about 150 nucleotides in length, preferably 12 to 50 nt, more preferably 15 and 45 nt in length.

290. The composition of any one of claims 287-289, wherein the target sequence comprises a target adjacent motif (TAM) sequence 3′ of the target sequence.

291. The composition of any of claims 287-290, wherein the target polynucleotide is DNA.

292. The composition of any of claims 287-291, wherein the guide nucleic acid further comprises an aptamer.

293. The composition of any of claims 287-292, wherein the guide nucleic acid further comprises an extension to add an RNA template.

294. The composition of any of claims 287-293, further comprising a functional domain associated with the IscB protein.

295. The composition of claim 294, wherein the functional domain has transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, detectable activity, or any combination thereof.

296. The composition of claim 294 or 295, wherein the functional domain is covalently linked with the IscB protein.

297. The composition of claim 296, wherein the IscB is fusion protein of any one of claims 32-45

298. The composition of any one of claims 287-297, further comprising a serine or tyrosine recombinase.

299. The composition of any one of claims 287-298, further comprising a homologous recombination donor template comprising a donor sequence for insertion into a target polynucleotide.

300. The composition of any one of claims 287-299, wherein a nuclease domain of the IscB protein is catalytically inactive.

301. The composition of any one of claims 287-300, wherein the nuclease domain has nickase activity or is engineered to have nickase activity.

302. The composition of any one of claims 287-301, wherein the target sequence comprises a TAM sequence of ATR, where R is purine (e.g., G or A).

303. An engineered cell comprising the composition of any one of claims 287-302.

304. A method of modifying a target sequence in a cell, the method comprising administering a composition of any of claims 287-302 to the cell.

305. A method of modifying a target sequence in a cell, the method comprising administering to the cell a composition comprising an IscB protein of any of claims 1 to 31 and/or a guide nucleic acid comprising the ωRNA of any one of claims 46-174, wherein IscB protein and/or the guide nucleic acid are provided via one or more polynucleotides encoding the IscB and/or guide nucleic acid, and wherein the one or more polynucleotides are operably configured to express the IscB/or the guide nucleic acid.

306. The method of claim 304 or 305, wherein the modifying comprises cleaving a DNA polynucleotide.

307. The method of any of claims 304-306, wherein the cleaving results in 5′ overhangs.

308. A method of targeting a polynucleotide, comprising contacting a sample that comprises a target polynucleotide with the composition of any one of claims 287-302, or one or more polynucleotides encoding one or more components of the composition of any of claims 287-302.

309. The method of claim 308, wherein contacting results in modification of a gene product or modification of the amount or expression of a gene product.

310. The method of claim 309, wherein the target sequence of the polynucleotide is a disease-associated target sequence.

311. A composition comprising:
a. the IscB protein of any one of claims 1-31, wherein the IscB protein is catalytically inactive,
b. a nucleotide deaminase associated with or otherwise capable of forming a complex with the IscB protein, and
c. a guide nucleic acid capable of forming a complex with the IscB protein and directing site-specific binding at a target sequence.

312. A composition comprising:
a. the IscB protein of any one of claims 1-31, wherein the IscB protein is catalytically inactive,
b. a nucleotide deaminase associated with or otherwise capable of forming a complex with the IscB protein, and
c. a guide nucleic acid comprising the ωRNA of any one of claims 46-174.

313. The composition of claim 311 or 312, wherein the nucleotide deaminase is an adenosine deaminase or a cytidine deaminase.

314. A cell or progeny thereof genetically engineered to express one or more components of the composition of any one of claims 311-313.

315. A method of editing nucleic acids in target polynucleotides comprising delivering the composition of any one of claims 311-313, or one or more polynucleotides encoding one or more components of the composition, to a cell or population of cells comprising the target sequence.

316. The method of claim 315, wherein the target polynucleotides are target sequences within genomic DNA.

317. The method of claim 315 or 316, wherein the target polynucleotide is edited at one or more bases to introduce a G->A or C->T mutation.

318. An isolated cell or progeny thereof comprising one or more base edits made using the method of any one of claims 315-317.

319. A composition comprising:
a. the IscB protein of any one of claims 1-31, wherein the IscB is catalytically inactive,
b. a reverse transcriptase associated with or otherwise capable of forming a complex with the IscB protein, and
c. a guide nucleic acid capable of forming a complex with the IscB protein and directing site-specific binding of the IscB to a target sequence, and wherein the guide nucleic further comprises a donor sequence for insertion into the target sequence.

320. A composition comprising:
a. the IscB protein of any one of claims 1-31, wherein the IscB is catalytically inactive,
b. a reverse transcriptase associated with or otherwise capable of forming a complex with the IscB protein, and
c. a guide nucleic acid comprising the ωRNA of any one of claims 46-174.

321. A method of modifying target polynucleotides comprising delivering the composition of claim 319 or 320, or one or more polynucleotides encoding one or more components of the composition to a cell, wherein the complex directs the reverse transcriptase to the target sequence and the reverse transcriptase facilitates insertion of the donor sequence from the guide nucleic acid into the target sequence

322. The method of claim 321, wherein insertion of the donor sequence: introduces one or more base edits; corrects or introduces a premature stop codon; disrupts a splice site; inserts or restores a splice site; inserts a gene or gene fragment at one or both alleles of the target polynucleotide; or a combination thereof.

323. An isolated cell or progeny thereof comprising the modifications made using the method of claim 321 or 322.

324. A composition comprising:
a. the IscB protein of any one of claims 1-31;
b. a non-LTR retrotransposon protein associated with or otherwise capable of forming a complex with the IscB protein;
c. guide nucleic acid capable of forming a complex with the IscB protein and directing site-specific binding to a target sequence; and
d. a donor construct comprising a donor polynucleotide for insertion to the target sequence and located between two binding elements capable of forming a complex with the non-LTR retrotransposon protein.

325. A composition comprising:
a. the IscB protein of any one of claims 1-31;
b. a non-LTR retrotransposon protein associated with or otherwise capable of forming a complex with the IscB protein;
c. a guide nucleic acid comprising the ωRNA of any one of claims 46-174; and
d. a donor construct comprising a donor polynucleotide for insertion to the target sequence and located between two binding elements capable of forming a complex with the non-LTR retrotransposon protein.

326. The composition of claim 324 or 325, wherein the IscB protein is fused to the N-terminus of the non-LTR retrotransposon protein.

327. The composition of any one of claims 324-326, wherein the IscB protein is engineered to have nickase activity.

328. The composition of claim 327, wherein the guide nucleic acid directs the fusion protein to a target sequence 5′ of the targeted insertion site, and wherein the IscB protein generates a double-strand break at the targeted insertion site.

329. The composition of claim 327, wherein the guide nucleic acid directs the fusion protein to a target sequence 3′ of the targeted insertion site, and wherein the IscB protein generates a double-strand break at the targeted insertion site.

330. The composition of any one of claims 324-329, wherein the donor polynucleotide further comprises a polymerase processing element to facilitate 3′ end processing of the donor polynucleotide sequence.

331. The composition of any one of claims 324-330, wherein the donor polynucleotide further comprises a homology region to the target sequence on the 5′ end of the donor construct, the 3′ end of the donor construct, or both.

332. The composition of any one of claims 324-331, wherein the homology region is from 8 to 25 base pairs.

333. A method of modifying target polynucleotides comprising delivering the composition of any one of claims 324-332, or one or more polynucleotides encoding one or more components of the composition to a cell, wherein the complex directs the non-LTR retrotransposon protein to the target sequence and the non-LTR retrotransposon protein facilitates insertion of the donor polynucleotide sequence from the donor construct into the target polynucleotide.

334. The method of claim 333, wherein insertion of the donor sequence: introduces one or more base edits; corrects or introduces a premature stop codon; disrupts a splice site; inserts or restores a splice site; inserts a gene or gene fragment at one or both alleles of the target polynucleotide; or a combination thereof.

335. An isolated cell or progeny thereof comprising the modifications made using the method of claim 333 or 334.

336. A method of modifying a target nucleic acid, the method comprising contacting a DNA and RNA targeting system or composition of any one of claim 185-209 or 287-302.

337. The method of claim 336, wherein the target nucleic acid is in a cell and the method comprises administering to the cell a DNA and RNA targeting system or composition of any one of claim 185-209 or 287-302.

338. The method of any one of claims 336-337, wherein said modifying the target nucleic acid comprises cleaving the target nucleic acid.

339. The method of claim 338, wherein said cleaving the target nucleic acid forms at least one 5′-overhang.

340. The method of claim 338 or 339, wherein said modifying the target nucleic acid comprises deletion, insertion or change of a nucleotide in the target nucleic acid.

341. The method of any one of claims 336-340, wherein said modifying the target nucleic acid comprises editing (e.g., changing) one or more nucleobases; correcting or introducing a premature stop codon, disrupting a splice site; inserting or restoring a splice site, inserting a gene or gene fragment into the target nucleic acid, or a combination thereof.

342. The method of any one of claims 336-341, wherein the target nucleic acid is DNA.

343. The method of claim 342, wherein the target nucleic acid is genomic DNA, episomal DNA, viral DNA, or bacterial DNA.

344. The method of claim 343, wherein the target nucleic acid is double-stranded DNA.

345. The method of claim 343, wherein the target nucleic acid is single-stranded DNA.

346. The method of any one of claims 336-341, wherein the target nucleic acid is RNA.

347. The method of claim 346, wherein the target nucleic acid is double-stranded RNA.

348. The method of claim 346, wherein the target nucleic acid is single-stranded RNA.

349. The method of claim 346, wherein the target nucleic acid is RNA selected from the group consisting of messenger RNA (mRNA), pre-mRNA, ribosomal RNA (rRNA), transfer RNA (tRNA), micro-RNA (miRNA), small nuclear RNA (snRNA), small nucleolar RNA (snoRNA), non-coding RNA (ncRNA), long non-coding RNA (lncRNA), and small cytoplasmatic RNA (scRNA).

350. The method of any one of claims 336-349, wherein the target nucleic acid is a disease-associated nucleic acid sequence.

351. An isolated nucleic acid modified by a method of anyone of claims 336-349.

352. An isolated cell or progeny thereof comprising a nucleic acid modified by a method of anyone of claims 336-349.
